(19) United States
(12) Patent Application Publication
(10) Pub. No.: US 2026/0125375 A1
(43) Pub. Date: May 7, 2026

(54) SUBSTITUTED 1H-PYRAZOLO-PYRIDINE AND -PYRIMIDINE COMPOUNDS

(71) Applicant: Pfizer Inc., New York, NY (US)

(72) Inventors: Sujin Cho-Schultz, San Diego, CA (US); Judith Gail Deal, Wildomar, CA (US); Joyann Susan Donaldson, Poway, CA (US); Kevin Daniel Freeman-Cook, Carlsbad, CA (US); Mehran Jalaie, San Diego, CA (US); Matthew Scott Jeffreys, Carlsbad, CA (US); Stephen Elliott Kaiser, San Diego, CA (US); John Charles Kath, La Mesa, CA (US); Timothy Patrick Montgomery, San Diego, CA (US); Sajiv Krishnan Nair, Vista, CA (US); Ryan Lloyd Patman, San Diego, CA (US); Khanh Tuan Tran, San Diego, CA (US); Hanna Maria Wisniewska, San Diego, CA (US); Ru Zhou, Carlsbad, CA (US)

(73) Assignee: Pfizer Inc., New York, NY (US)

(21) Appl. No.: 19/117,817

(22) PCT Filed: Oct. 2, 2023
(86) PCT No.: PCT/IB2023/059856

§ 371 Date: Apr. 2, 2025

Related U.S. Application Data
(60) Provisional application No. 63/378,283, filed on Oct. 4, 2022.
(60) Provisional application No. 63/479,647, filed on Jan. 12, 2023.
(60) Provisional application No. 63/583,684, filed on Sep. 19, 2023.

(51) Int. Cl. C07D 471/04; A61K 31/4196; A61K 31/437; A61K 31/4545; A61K 31/496; A61K 31/519; A61K 31/5377; A61K 45/06; C07D 405/14; C07D 487/04

(52) U.S. Cl. CPC C07D 471/04; A61K 31/4196; A61K 31/437; A61K 31/4545; A61K 31/496; A61K 31/519; A61K 31/5377; A61K 45/06; C07D 405/14; C07D 487/04

(57) ABSTRACT

This disclosure relates to compounds of Formula (I) (I), or a pharmaceutically acceptable salt thereof, in which R3, R4, L2, W, X, Y, and Z are as 5 defined herein, to pharmaceutical compositions comprising such compounds and salts; to processes for their preparation; to intermediates used in such process; and to methods of using such compounds, salts and compositions for the treatment of immunosuppression-associated disorders such as chronic viral infections and cancer.

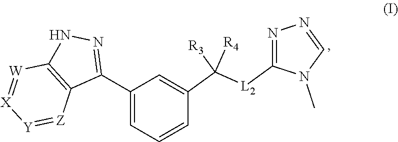

### FIELD

The present disclosure relates to novel 1H-pyrazolo-pyridine and -pyrimidine compounds. The disclosure also relates to the preparation of the compounds and intermediates used in the preparation, compositions containing the compounds, and uses of the compounds as inhibitors of the E3 ubiquitin ligase enzyme, casitas B-lineage lymphoma proto-oncogene-b (CBL-B), in the treatment of immunosuppression-associated disorders such as chronic viral infections and cancer.

### BACKGROUND

CBL-B is an E3 ubiquitin ligase and a key negative feedback regulator of TCR and costimulatory receptor signal transduction, which directly binds to and ubiquitinates multiple substrates including zeta-chain-associated protein kinase 70 (Zap70) (Zhang et al., Current Biology, 1999, 9(4):203-206, PMID 10074432) and p85 (Fang et al., Nature Immunology, 2001, PMID 11526404). Genetic loss of CBL-B catalytic function (CD3+ T cells that lacked CBL-B E3 ubiquitin ligase activity, C373A KI/KI cells) overcomes the requirement for co-stimulation provided by antigen presenting cells, decreases the T cell activation threshold and renders T cells resistant to repeat stimulation induced anergy. Consequently, loss of CBL-B activity leads to spontaneous tumor clearance in syngeneic tumor models in a CD8 T cell-dependent manner. (Paolino et al., J. Immunol. 2011, 186(4): 2138-2147, PMID 21248250).

CBL-B is thus a compelling target for modulating an immune response to cancer. One such opportunity is to promote signaling via the T cell receptor (TCR) (Hwang et al., Exp. Mol. Med. 2020, 52(5):750-761, PMID 32439954) to enhance T cell activation. Following engagement of the TCR by major histocompatibility (MHC)-antigen complexes, a well described signal transduction cascade ensues, leading to the phosphorylation of substrates including Lck and Zap70 ultimately leading to the production of inflammatory cytokines (IL-2, IFNg and TNFa) and the acquisition of cytotoxic effector function.

While CBL-B inhibition is a compelling therapeutic strategy, there are possible adverse autoimmune risks associated with simultaneous inhibition of the closely related molecule casitas B lineage lymphoma (C-CBL). The ubiquitously expressed protein C-CBL plays an important role in the internalization and negative regulation of signaling from receptor tyrosine kinases including epidermal growth factor receptor (EGFR) (Yokouchi et al., J Biol. Chem. 1999, 274(44):31707-12, PMID 10531381). Mice containing T-cells that are deficient for both CBL-B and C-CBL demonstrate a hypersensitive immune activation phenotype and succumb to lethal autoimmunity 12-14 weeks after birth, and mice with global loss of both CBL-B and C-CBL succumb to a systemic myeloproliferative disorder within a similar timeframe. (Naramura et al., Proc. Natl. Acad. Sci. 2010, 107(37):16274-9, PMID 20805496). These findings highlight the potential therapeutic benefit of inhibitors that display selectivity for CBL-B over C-CBL with broad application in cancer immunotherapy.

Accordingly, there remains a need for improved therapeutic strategies to enhance anti-tumor immunity in patients.

### SUMMARY OF THE DISCLOSURE

The present disclosure provides, in part, compounds of Formulae (I) to (V), including sub-Formulae (II-a to II-f), and pharmaceutically acceptable salts thereof. The compounds of the present disclosure may inhibit the activity of CBL-B and may be useful in the treatment, prevention, suppression and amelioration of cancer (see, for example, Chiang et al., J. Clin. Invest. 2007,117(4):1029-36, PMID 17364027), chronic viral infection (Ou et al., J. Virol. 2008, 82(7):3353-68, PMID 18199651) or diseases, disorders and conditions mediated by CBL-B. In particular, such compounds show affinity/activity for CBL-B which is greater than their affinity/activity against C-CBL. Also provided are pharmaceutical compositions, comprising the compounds or salts of the disclosure, alone or in combination with additional anticancer therapeutic agents. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the disclosure there is provided a compound of Formula (I)

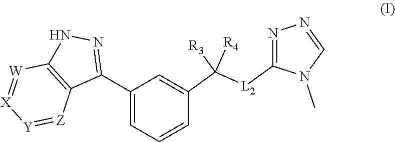

or a pharmaceutically acceptable salt thereof,
wherein:
   W is N or C-L₁-R₁;
   X is N or C-L₁-R₁;
   Y is N or CR₂;
   Z is N or CH;
   with the provisos that:
      one of W and X is N or CH and the other one of W and X is C-L₁-R₁;
      when Z is N, X is N; and
      Y and Z are not both N;
   R₁ is selected from the group consisting of H, halogen, C₃-C₁ cycloalkyl, 4-8 membered heterocycloalkyl, NR₅R₆, and OR₇; wherein said C₃-C₁₀ cycloalkyl or said 4-8 membered heterocycloalkyl ring is optionally substituted by one or more R₉ which can be the same or different;
   R₂ is selected from the group consisting of H, halogen, C₁-C₆ alkyl, C₁-C₆ haloalkyl, C₁-C₆ alkoxy and C₃-C₁₀ cycloalkyl; wherein said C₁-C₆ alkyl, said C₁-C₆ alkoxy or said C₃-C₁₀ cycloalkyl is optionally substituted by one or more halogen or oxo;
   R₃ and R₄ are independently selected from the group consisting of H, fluoro, C₁-C₆ alkyl, C₃-C₁₀ cycloalkyl and 4-8 membered heterocycloalkyl, each of which is optionally substituted by one or more R₁₀ which can be the same or different; or R₃ and R₄ together with the carbon atom to which they are attached form a C₃-C₈ cycloalkyl or 4-8 membered heterocycloalkyl ring, each of which is optionally substituted by one or more R₁₀ which can be the same or different;
   R₅ and R₆ are independently H, C(O)R₈, C(O)OR₈, C(O)NHR₈, SOR₈, SO₂R₈, C₁-C₆ alkyl, C₃-C₁₀ cycloalkyl, 4-8 membered heterocycloalkyl, wherein said C₁-C₆ alkyl, C₃-C₁₀ cycloalkyl or said 4-8 membered heterocycloalkyl is optionally substituted by one or more R₉ which can be the same or different; or R₅ and R₆ together with the nitrogen atom to which they are attached form a 4-8 membered heterocycloalkyl ring optionally further containing one or more heteroatoms selected from oxygen, sulfur, and nitrogen, wherein said 4-8 membered heterocycloalkyl ring is optionally substituted by one or more R₉ which can be the same or different;
   R₇ is H, C₁-C₆ alkyl or 4-8 membered heterocycloalkyl, wherein said C₁-C₆ alkyl or said C₃-C₁ heterocycloalkyl is optionally substituted by one or more R₉ which can be the same or different;
   R₈ is C₁-C₆ alkyl optionally substituted by one or more R₉ which can be the same or different;
   R₉ is each independently halogen, C₁-C₆ alkyl, OH, hydroxyalkyl, C₁-C₃ alkoxy, NR₁₁R₁₂, NR₁₁COR₁₂, NR₁₁SO₂R₁₂, NR₁₁COOR₁₂, NR₁₁CONR₁₂R₁₃, SR₁₁, SOR₁₁, SO₂R₁₁, CONR₁₁R₁₂, CN or oxo;
   R₁₀ is each independently halogen, C₁-C₃ alkyl, C₁-C₃ alkoxy, fluoroalkyl, CN, oxo or OH;
   R₁₁, R₁₂ and R₁₃ are each independently H or C₁-C₃ alkyl; and
   L₁ and L₂ are independently a bond or a C₁-C₃ alkylene optionally substituted by one or more F, C₁-C₃ alkyl, C₁-C₃ alkoxy, fluoroalkyl or OH.

Described below are embodiments of the disclosure, where for convenience Embodiment 1 (E1) is identical to the embodiment of Formula (I) provided above.

It is to be understood that both the foregoing general description and the following detailed description are exemplary and explanatory only and are not restrictive of the disclosure, as claimed.

### DETAILED DESCRIPTION OF THE DISCLOSURE

The present disclosure may be understood more readily by reference to the following detailed description of the embodiments of the disclosure and the Examples included herein. It is to be understood that this disclosure is not limited to specific synthetic methods of making that may of course vary. It is to be also understood that the terminology used herein is for the purpose of describing specific embodiments only and is not intended to be limiting.
E1 A compound of Formula (I) or a pharmaceutically acceptable salt thereof, as defined above.
E2 A compound or pharmaceutically acceptable salt of embodiment E1, wherein

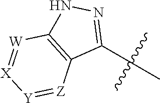

is selected from the group consisting of:

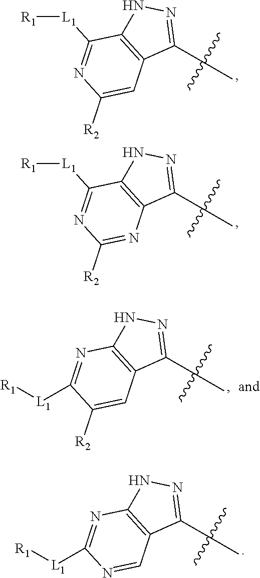

E3 A compound of Formula (II), (Ill), (IV), or (V):

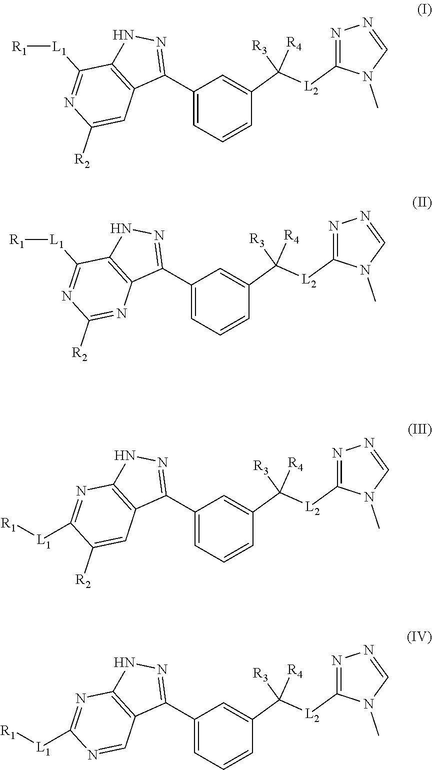

or a pharmaceutically acceptable salt thereof, wherein R₁, R₂, R₃, R₄, L₁ and L₂ are defined as for Formula (I). Each of the aspects and embodiments described herein with respect to Formula (I) is also applicable to compounds of Formula (II), (Ill), (IV) or (V).
E4 A compound of Formula (II):

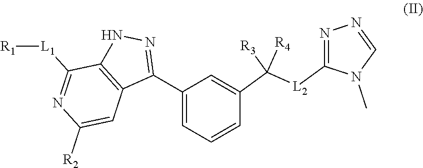

or a pharmaceutically acceptable salt thereof, wherein R₁, R₂, R₃, R₄, L₁ and L₂ are defined as for Formula (I).
E5 A compound of Formula (II-a), (II-b), (II-c), (II-d), (II-e), or (11-f):

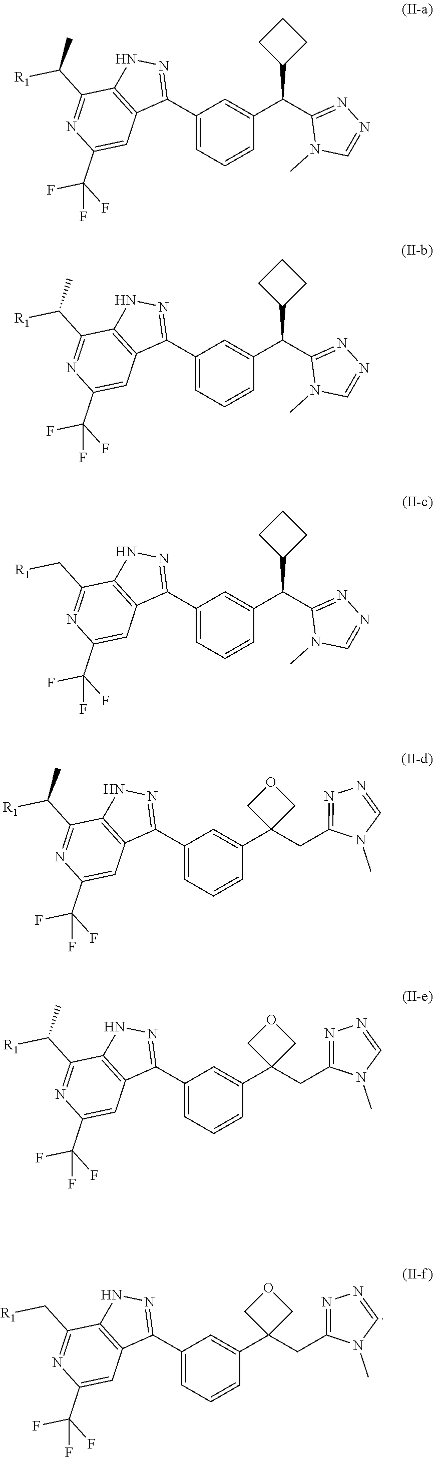

or a pharmaceutically acceptable salt thereof,
wherein R₁ is defined as for Formula (II) above in embodiment E4.
E6 The compound or pharmaceutically acceptable salt of any one of embodiments E1-E5, wherein R₁ is H.
E7 The compound or pharmaceutically acceptable salt of any one of embodiments E1-E5, wherein R₁ is C₃-C₁₀ cycloalkyl or 4-8 membered heterocycloalkyl, wherein said C₃-C₁₀ cycloalkyl or said 4-8 membered heterocycloalkyl is optionally substituted by one or more R₉ (e.g., optionally substituted by 0, 1, 2, or 3 R₉) which can be the same or different.
E8 The compound or pharmaceutically acceptable salt of any one of embodiments E1-E5, wherein R₁ is NR₅R₆.
E9 The compound or pharmaceutically acceptable salt of any one of embodiments E1-E5, wherein R₅ and R₆ are each independently C₁-C₆ alkyl which can be the same or different.
E10 The compound or pharmaceutically acceptable salt of any one of embodiments E1-E5 and E8, wherein one of R₅ and R₆ is H and the other one of R₅ and R₆ is C₁-C₆ alkyl, C₃-C₁₀ cycloalkyl, 4-8 membered heterocycloalkyl, C(O)R₈, C(O)OR₈, C(O)NHR₈, SOR₈, or SO₂R₈, wherein said C₁-C₆ alkyl, said C₃-C₁₀ cycloalkyl, or said 4-8 membered heterocycloalkyl is optionally substituted by one or more R₉ (e.g., optionally substituted by 0, 1, 2, or 3 R₉) which can be the same or different.
E11 The compound or pharmaceutically acceptable salt of any one of embodiments E1-E5, E8, and E10, wherein one of R₅ and R₆ is selected from the group consisting of C₁-C₃ alkyl, cyclopropyl, cyclobutyl, cyclopentyl, tetrahydrofuranyl, and azetidinyl, where each of which is optionally substituted by one or more R₉ (e.g., optionally substituted by 0, 1, 2, or 3 R₉) which can be the same or different.
E12 The compound or pharmaceutically acceptable salt of any one of embodiments E1-E5 and E8, wherein R₅ and R₆ together with the nitrogen atom to which they are attached form a 4-8 membered heterocycloalkyl ring optionally further containing one or more groups selected from oxygen, sulfur, and nitrogen.
E13 The compound or pharmaceutically acceptable salt of embodiment E12, wherein said 4-8 membered heterocycloalkyl ring is unsubstituted.
E14 The compound or pharmaceutically acceptable salt of embodiment E12, wherein said 4-8 membered heterocycloalkyl ring is selected from the group consisting of:

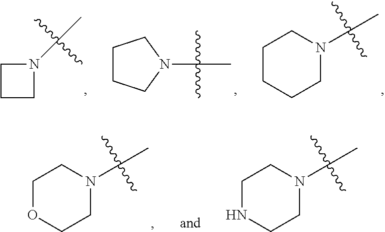 [SMILES: *N1CCC1 ; *N1CCCC1 ; *N1CCCCC1 ; *N1CCOCC1 ; *N1CCNCC1]

optionally substituted by one or more R₉ (e.g., optionally substituted by 0, 1, 2, or 3 R₉) which can be the same or different.
E15 The compound or pharmaceutically acceptable salt of any one of embodiments E1-E5, E8 and E10, wherein R₈ is C₁-C₆ alkyl optionally substituted by one or more R₉ (e.g., optionally substituted by 0, 1, 2, or 3 R₉) which can be the same or different.
E16 The compound or pharmaceutically acceptable salt of any one of embodiments E1-E5, wherein R₁ is OR₇.
E17 The compound or pharmaceutically acceptable salt of any one of embodiments E1-E5 and E16, wherein R₇ is C₁-C₆ alkyl or 4-8 membered heterocycloalkyl, wherein said C₁-C₆ alkyl or 4-8 membered heterocycloalkyl is optionally substituted by one or more R₉ (e.g., optionally substituted by 0, 1, 2, or 3 R₉) which can be the same or different.
E18 The compound or pharmaceutically acceptable salt of any one of embodiments E1-E17, wherein each R₉ is independently halogen, OH, NH₂, C₁-C₆ alkyl, or oxo.
E19 The compound or pharmaceutically acceptable salt of any one of embodiments E1-E18, wherein each R₉ is independently F, OH, or methyl.
E20 The compound or pharmaceutically acceptable salt of any one of embodiments E1-E19, wherein R₂ is optionally substituted by one or more F (e.g., optionally substituted by 0, 1, 2, or 3 F).
E21 The compound or pharmaceutically acceptable salt of any one of embodiments E1-E20, wherein R₂ is —CF₃.
E22 The compound or pharmaceutically acceptable salt of any one of embodiments E1-E21, wherein R₃ and R₄ are independently H, F or C₁-C₆ alkyl.
E23 The compound or pharmaceutically acceptable salt of any one of embodiments E1-E21, wherein one of R₃ and R₄ is H, and the other one of R₃ and R₄ is C₁-C₆ alkyl, 4-8 membered heterocycloalkyl, or C₃-C₆ cycloalkyl, wherein said C₁-C₆ alkyl, said 4-8 membered heterocycloalkyl, or said C₃-C₆ cycloalkyl is optionally substituted by one or more R₁₀ (e.g., optionally substituted by 0, 1, 2, or 3 R₁₀) which can be the same or different.
E24 The compound or pharmaceutically acceptable salt of any one of embodiments E1-E23, wherein R₃ is C₁-C₆ alkyl.
E25 The compound or pharmaceutically acceptable salt of any one of embodiments E1-E21 and E23, wherein R₃ is 4-8 membered heterocycloalkyl.
E26 The compound or pharmaceutically acceptable salt of any one of embodiments E1-E21 and E23, wherein R₃ is C₃-C₆ cycloalkyl.
E27 The compound or pharmaceutically acceptable salt of any one of embodiments E1-E21, E23 and E26, wherein R₃ is cyclopropyl, cyclobutyl, cyclopentyl, cyclohexyl, bicyclo[1.1.1]pentyl, or bicyclo[2.1.1]hexanyl.
E28 The compound or pharmaceutically acceptable salt of any one of embodiments E1-E21, E23, E26 and E27, wherein R₃ is cyclobutyl.
E29 The compound or pharmaceutically acceptable salt of any one of embodiments E1-E28, wherein R₃ is substituted by one or more (e.g., substituted by one or two) C₁-C₃ alkyl, halogen, OH, and/or oxo.
E30 The compound or pharmaceutically acceptable salt of any one of embodiments E1-E29, R₃ is cyclobutyl substituted by methyl, F, OH, oxo, or combinations thereof.
E31 The compound or pharmaceutically acceptable salt of any one of embodiments E1-E23, E26-E30, wherein R₃ is cyclobutyl substituted by one or more methyl and/or F.
E32 The compound or pharmaceutically acceptable salt of any one of embodiments E1-E23, E26-E28, wherein R₃ is unsubstituted cyclobutyl.
E33 The compound or pharmaceutically acceptable salt of any one of embodiments E1-E21, wherein R₃ and R₄ together with the carbon atom to which they are attached to form a 4-8 membered heterocycloalkyl ring.
E34 The compound or pharmaceutically acceptable salt of any one of embodiments E1-E20 and E33, wherein R₃ and R₄ together with the carbon atom to which they are attached to form an oxetane optionally substituted by one or more substituent(s) selected from methyl, F, and combinations thereof.
E35 The compound or pharmaceutically acceptable salt of any one of embodiments E1-E20, E33 and E34, wherein R₃ and R₄ together with the carbon atom to which they are attached to form an unsubstituted oxetane.
E36 The compound or pharmaceutically acceptable salt of any one of embodiments E1-E35, wherein L₁ is C₁-C₃ alkylene optionally substituted by one or two substituents selected from F, C₁-C₃ alkyl, C₁-C₃ alkoxy, fluoroalkyl, OH or combinations thereof.
E37 The compound or pharmaceutically acceptable salt of any one of embodiments E1-E36, wherein L₁ is C₁-C₃ alkylene optionally substituted by F, methyl, methoxy, CF₃, OH or combinations thereof.
E38 The compound or pharmaceutically acceptable salt of any one of embodiments E1-E37, wherein L₁ is methylene or ethylene, optionally substituted by one or two methyl groups.
E39 The compound or pharmaceutically acceptable salt of any one of embodiments E1-E38, wherein L₁ is unsubstituted C₁-C₃ alkylene.
E40 The compound or pharmaceutically acceptable salt of any one of embodiments E1-E38, wherein L₁ is selected from —CH₂—, —CHCH₃—, —CH₂CH₂—, —CH(CH₃)CH₂—, or —CH₂CH(CH₃)—.
E41 The compound or pharmaceutically acceptable salt of any one of embodiments E1-E38 wherein L₁ is —CH₂— or —CHCH₃—.
E42 The compound or pharmaceutically acceptable salt of any one of embodiments E1-E41, wherein L₂ is a bond.
E43 The compound or pharmaceutically acceptable salt of any one of embodiments E1-E41, wherein L₂ is selected from —CH₂— or —CHCH₃—.
E44 The compound or pharmaceutically acceptable salt of any one of embodiments E1-E41, wherein L₂ is unsubstituted C₁-C₃ alkylene.
E45 The compound or pharmaceutically acceptable salt of any one of embodiments E1-E41, wherein L₂ is a bond; R₃ is cyclobutyl; and R₄ is H.
E46 The compound or pharmaceutically acceptable salt of any one of embodiments E1-E41, wherein L₂ is CH₂; and R₃ and R₄ together with the carbon atom to which they are attached to form an oxetane.
E47 The compound or pharmaceutically acceptable salt of any one of embodiments E1-E5,
wherein:
   R₁ is NR₅R₆;
   R₃ and R₄ are independently selected from the group consisting of H, C₃-C₁ cycloalkyl and 4-8 membered heterocycloalkyl, each of which is optionally substituted by one or more R₁₀ which can be the same or different; or R₃ and R₄ together with the carbon atom to which they are attached form a C₃-C₈ cycloalkyl or 4-8 membered heterocycloalkyl ring, each of which is optionally substituted by one or more R₁₀ which can be the same or different;
   R₅ and R₆ are independently H, C₁-C₆ alkyl, C₃-C₁ cycloalkyl, 4-8 membered heterocycloalkyl, wherein said C₁-C₆ alkyl, C₃-C₁₀ cycloalkyl and 4-8 membered heterocycloalkyl are optionally substituted by one or more R₉; or R₅ and R₆ together with the nitrogen atom to which they are attached form a 4-8 membered heterocycloalkyl ring optionally further containing one or more groups selected from oxygen, sulfur, and nitrogen, wherein said 4-8 membered heterocycloalkyl ring is optionally substituted by one or more R₉ which can be the same or different;
   each R₉ is independently halogen, OH, NH₂, C₁-C₆ alkyl, or oxo;
   L₁ is methylene or ethylene optionally substituted by one or two methyl; and
   L₂ is selected from a bond, —CH₂— or —CHCH₃—.
E48 The compound of any one of embodiments E1 to E47, or a pharmaceutically acceptable salt thereof, wherein the compound is selected from the group consisting of:

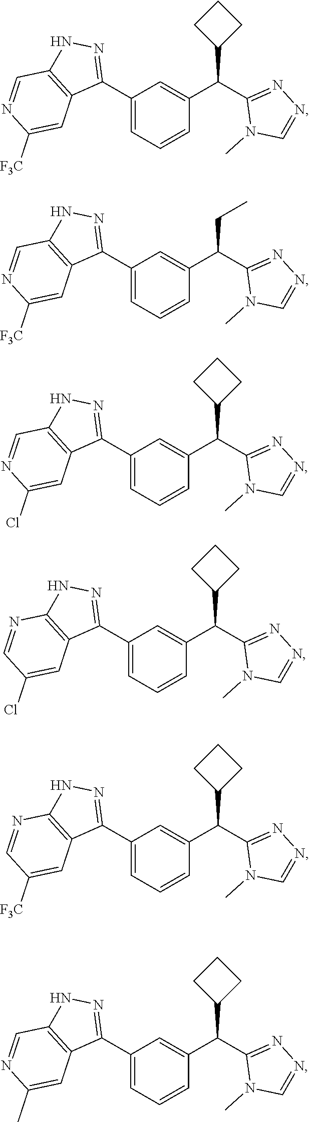

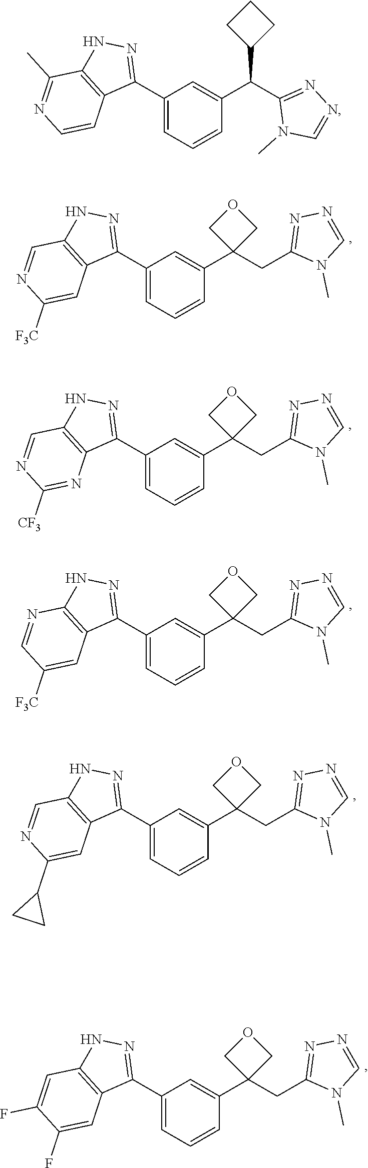 [SMILES: Cc1nccc2c(-c3cccc([C@H](c4nncn4C)C4CCC4)c3)n[nH]c12 ; Cn1cnnc1CC1(c2cccc(-c3n[nH]c4cnc(C(F)(F)F)cc34)c2)COC1 ; Cn1cnnc1CC1(c2cccc(-c3n[nH]c4cnc(C(F)(F)F)nc34)c2)COC1 ; Cn1cnnc1CC1(c2cccc(-c3n[nH]c4ncc(C(F)(F)F)cc34)c2)COC1 ; Cn1cnnc1CC1(c2cccc(-c3n[nH]c4cnc(C5CC5)cc34)c2)COC1 ; Cn1cnnc1CC1(c2cccc(-c3n[nH]c4cc(F)c(F)cc34)c2)COC1]

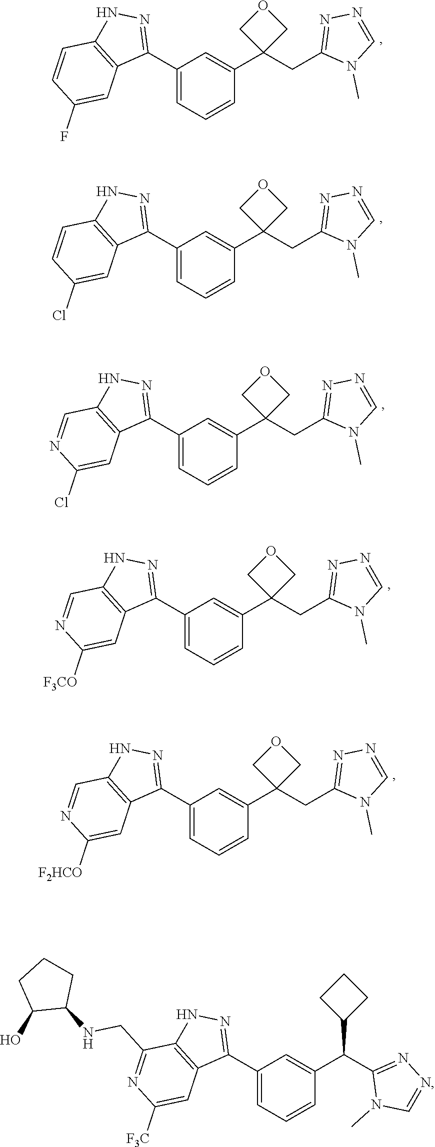 [SMILES: Cn1cnnc1CC1(c2cccc(-c3n[nH]c4ccc(F)cc34)c2)COC1 ; Cn1cnnc1CC1(c2cccc(-c3n[nH]c4ccc(Cl)cc34)c2)COC1 ; Cn1cnnc1CC1(c2cccc(-c3n[nH]c4cnc(Cl)cc34)c2)COC1 ; Cn1cnnc1CC1(c2cccc(-c3n[nH]c4cnc(OC(F)(F)F)cc34)c2)COC1 ; Cn1cnnc1CC1(c2cccc(-c3n[nH]c4cnc(OC(F)F)cc34)c2)COC1 ; Cn1cnnc1[C@@H](c1cccc(-c2n[nH]c3c(CN[C@@H]4CCC[C@@H]4O)nc(C(F)(F)F)cc23)c1)C1CCC1]

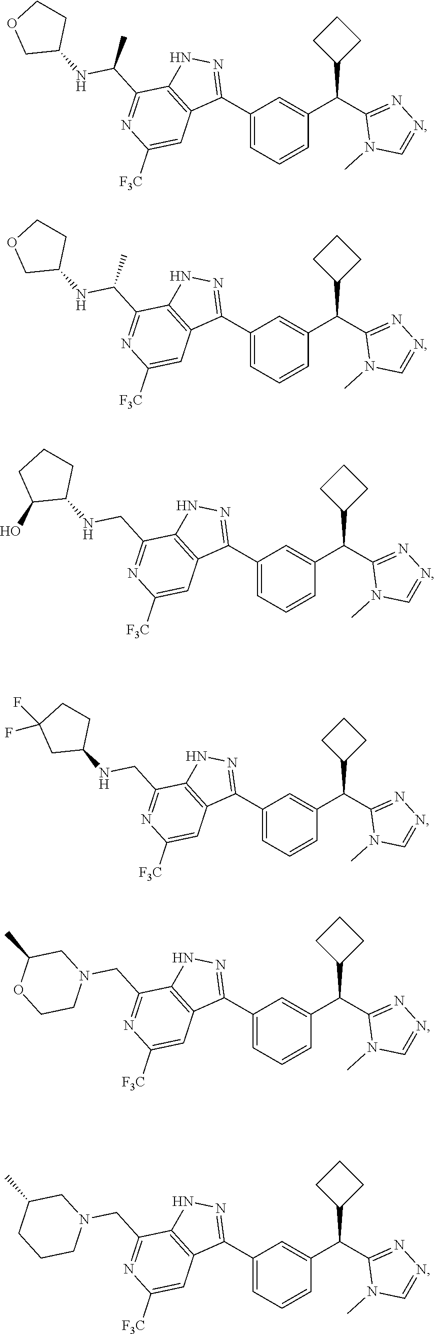 [SMILES: C[C@H](N[C@H]1CCOC1)c1nc(C(F)(F)F)cc2c(-c3cccc([C@H](c4nncn4C)C4CCC4)c3)n[nH]c12 ; C[C@@H](N[C@H]1CCOC1)c1nc(C(F)(F)F)cc2c(-c3cccc([C@H](c4nncn4C)C4CCC4)c3)n[nH]c12 ; Cn1cnnc1[C@@H](c1cccc(-c2n[nH]c3c(CN[C@H]4CCC[C@@H]4O)nc(C(F)(F)F)cc23)c1)C1CCC1 ; Cn1cnnc1[C@@H](c1cccc(-c2n[nH]c3c(CN[C@@H]4CCC(F)(F)C4)nc(C(F)(F)F)cc23)c1)C1CCC1 ; C[C@H]1CN(Cc2nc(C(F)(F)F)cc3c(-c4cccc([C@H](c5nncn5C)C5CCC5)c4)n[nH]c23)CCO1 ; C[C@H]1CCCN(Cc2nc(C(F)(F)F)cc3c(-c4cccc([C@H](c5nncn5C)C5CCC5)c4)n[nH]c23)C1]

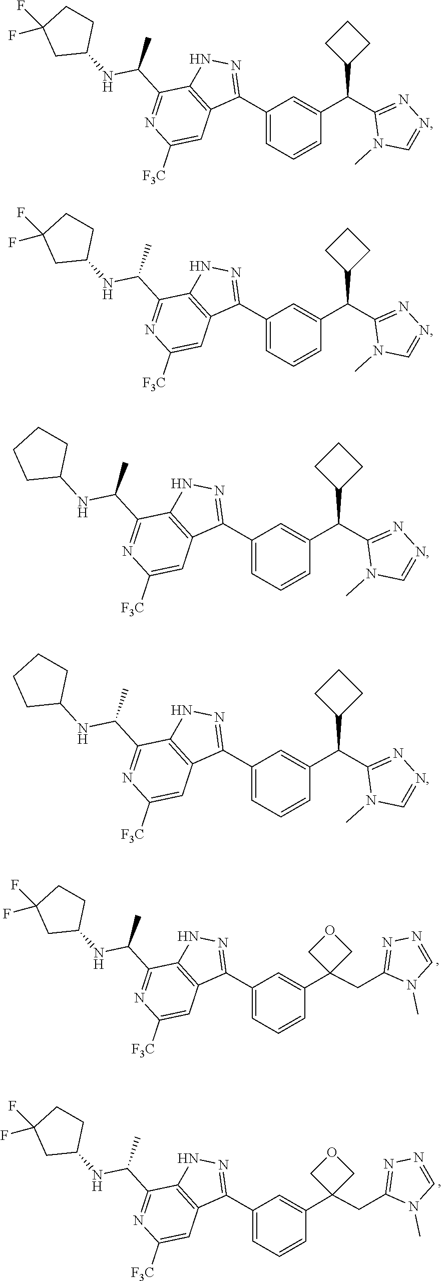

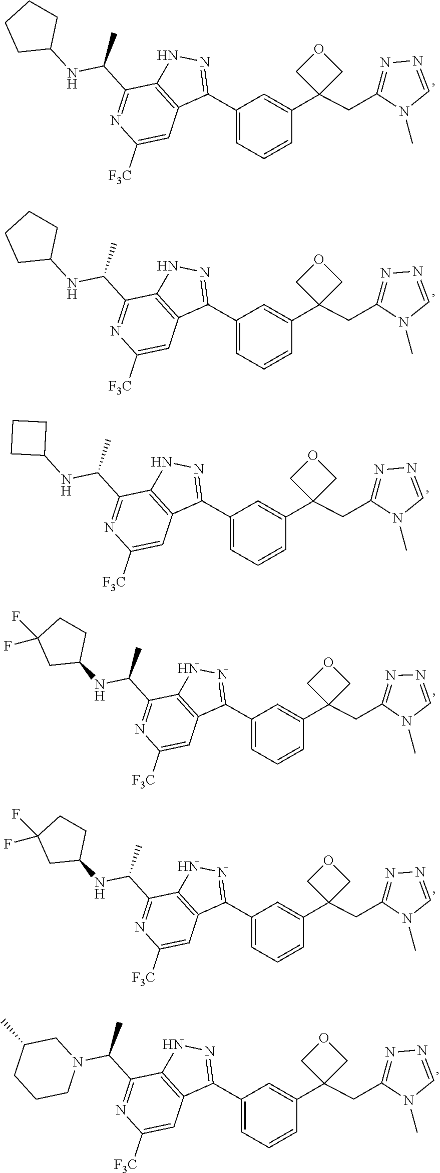 [SMILES: C[C@H](NC1CCCC1)c1nc(C(F)(F)F)cc2c(-c3cccc(C4(Cc5nncn5C)COC4)c3)n[nH]c12 ; C[C@@H](NC1CCCC1)c1nc(C(F)(F)F)cc2c(-c3cccc(C4(Cc5nncn5C)COC4)c3)n[nH]c12 ; C[C@@H](NC1CCC1)c1nc(C(F)(F)F)cc2c(-c3cccc(C4(Cc5nncn5C)COC4)c3)n[nH]c12 ; C[C@H](N[C@@H]1CCC(F)(F)C1)c1nc(C(F)(F)F)cc2c(-c3cccc(C4(Cc5nncn5C)COC4)c3)n[nH]c12 ; C[C@@H](N[C@@H]1CCC(F)(F)C1)c1nc(C(F)(F)F)cc2c(-c3cccc(C4(Cc5nncn5C)COC4)c3)n[nH]c12 ; C[C@H]1CCCN([C@@H](C)c2nc(C(F)(F)F)cc3c(-c4cccc(C5(Cc6nncn6C)COC5)c4)n[nH]c23)C1]

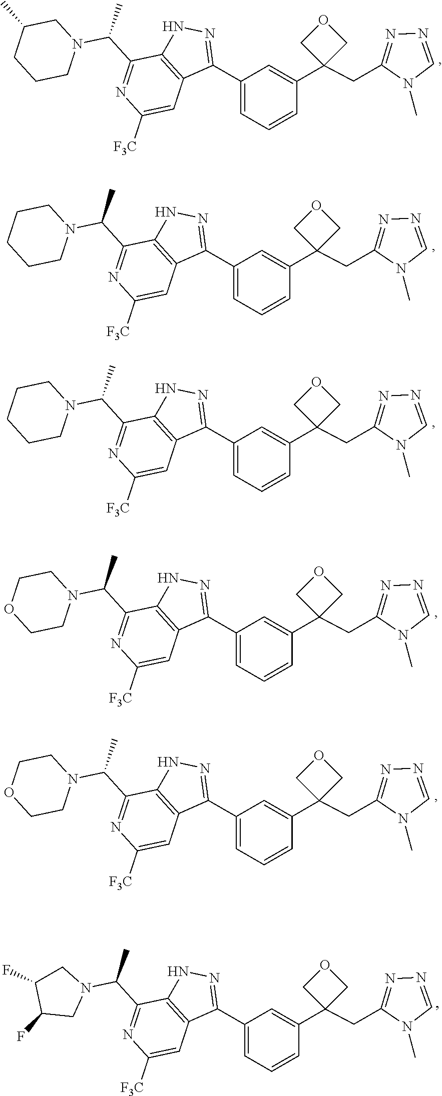

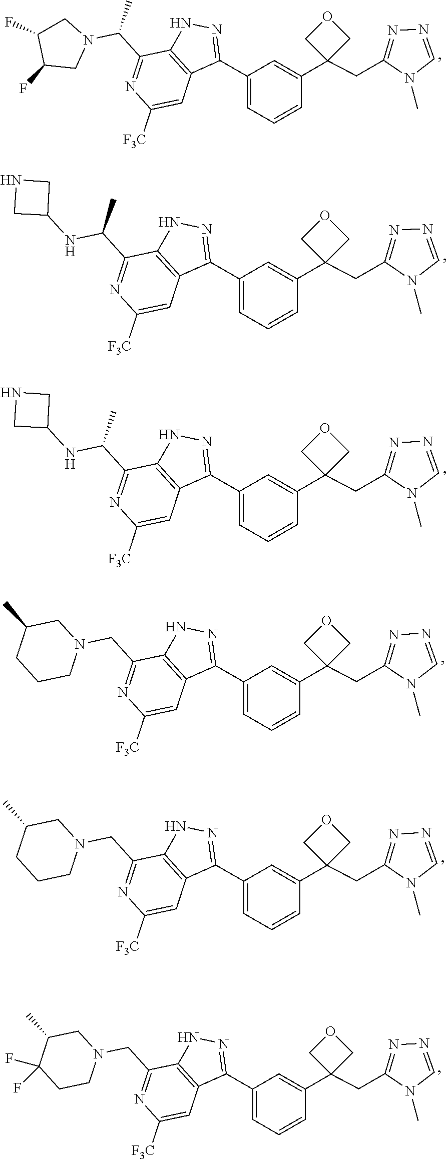 [SMILES: C[C@H](c1nc(C(F)(F)F)cc2c(-c3cccc(C4(Cc5nncn5C)COC4)c3)n[nH]c12)N1C[C@@H](F)[C@H](F)C1 ; C[C@H](NC1CNC1)c1nc(C(F)(F)F)cc2c(-c3cccc(C4(Cc5nncn5C)COC4)c3)n[nH]c12 ; C[C@@H](NC1CNC1)c1nc(C(F)(F)F)cc2c(-c3cccc(C4(Cc5nncn5C)COC4)c3)n[nH]c12 ; C[C@@H]1CCCN(Cc2nc(C(F)(F)F)cc3c(-c4cccc(C5(Cc6nncn6C)COC5)c4)n[nH]c23)C1 ; C[C@H]1CCCN(Cc2nc(C(F)(F)F)cc3c(-c4cccc(C5(Cc6nncn6C)COC5)c4)n[nH]c23)C1 ; C[C@@H]1CN(Cc2nc(C(F)(F)F)cc3c(-c4cccc(C5(Cc6nncn6C)COC5)c4)n[nH]c23)CCC1(F)F]

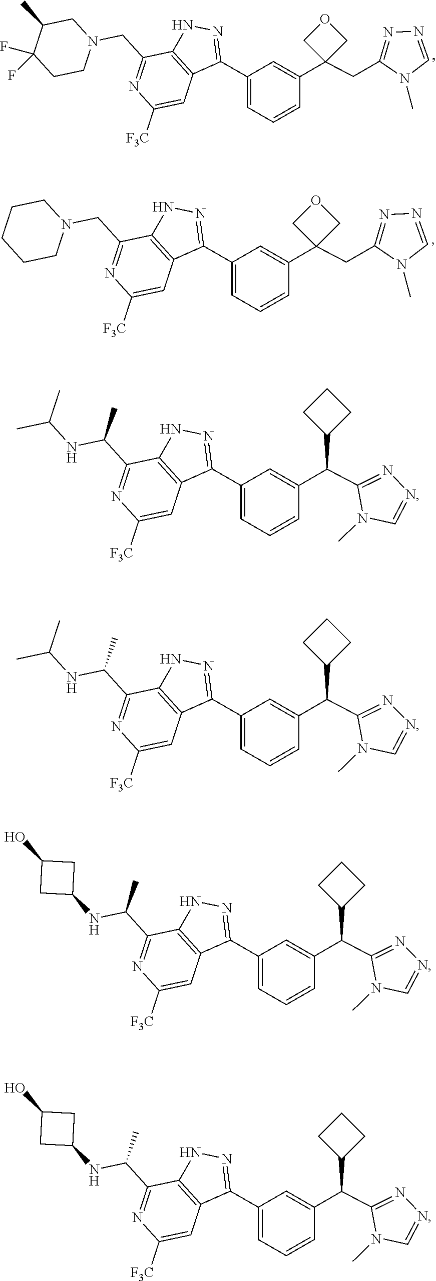 [SMILES: C[C@H]1CN(Cc2nc(C(F)(F)F)cc3c(-c4cccc(C5(Cc6nncn6C)COC5)c4)n[nH]c23)CCC1(F)F ; Cn1cnnc1CC1(c2cccc(-c3n[nH]c4c(CN5CCCCC5)nc(C(F)(F)F)cc34)c2)COC1 ; CC(C)N[C@@H](C)c1nc(C(F)(F)F)cc2c(-c3cccc([C@H](c4nncn4C)C4CCC4)c3)n[nH]c12 ; CC(C)N[C@H](C)c1nc(C(F)(F)F)cc2c(-c3cccc([C@H](c4nncn4C)C4CCC4)c3)n[nH]c12 ; C[C@H](N[C@H]1C[C@@H](O)C1)c1nc(C(F)(F)F)cc2c(-c3cccc([C@H](c4nncn4C)C4CCC4)c3)n[nH]c12 ; C[C@@H](N[C@H]1C[C@@H](O)C1)c1nc(C(F)(F)F)cc2c(-c3cccc([C@H](c4nncn4C)C4CCC4)c3)n[nH]c12]

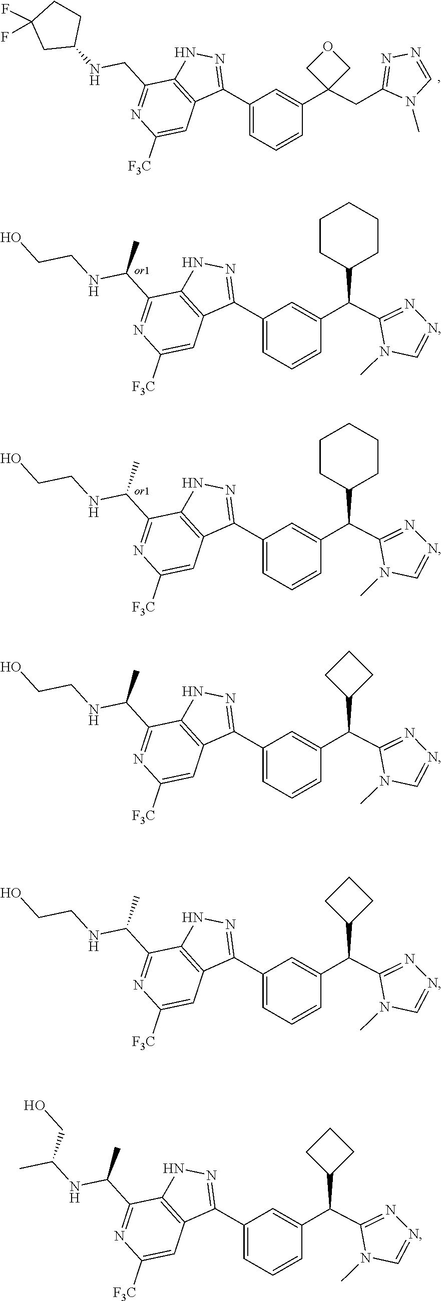 [SMILES: Cn1cnnc1CC1(c2cccc(-c3n[nH]c4c(CN[C@H]5CCC(F)(F)C5)nc(C(F)(F)F)cc34)c2)COC1 ; C[C@H](NCCO)c1nc(C(F)(F)F)cc2c(-c3cccc([C@H](c4nncn4C)C4CCCCC4)c3)n[nH]c12 ; C[C@@H](NCCO)c1nc(C(F)(F)F)cc2c(-c3cccc([C@H](c4nncn4C)C4CCCCC4)c3)n[nH]c12 ; C[C@H](NCCO)c1nc(C(F)(F)F)cc2c(-c3cccc([C@H](c4nncn4C)C4CCC4)c3)n[nH]c12 ; C[C@@H](NCCO)c1nc(C(F)(F)F)cc2c(-c3cccc([C@H](c4nncn4C)C4CCC4)c3)n[nH]c12 ; C[C@H](CO)N[C@@H](C)c1nc(C(F)(F)F)cc2c(-c3cccc([C@H](c4nncn4C)C4CCC4)c3)n[nH]c12]

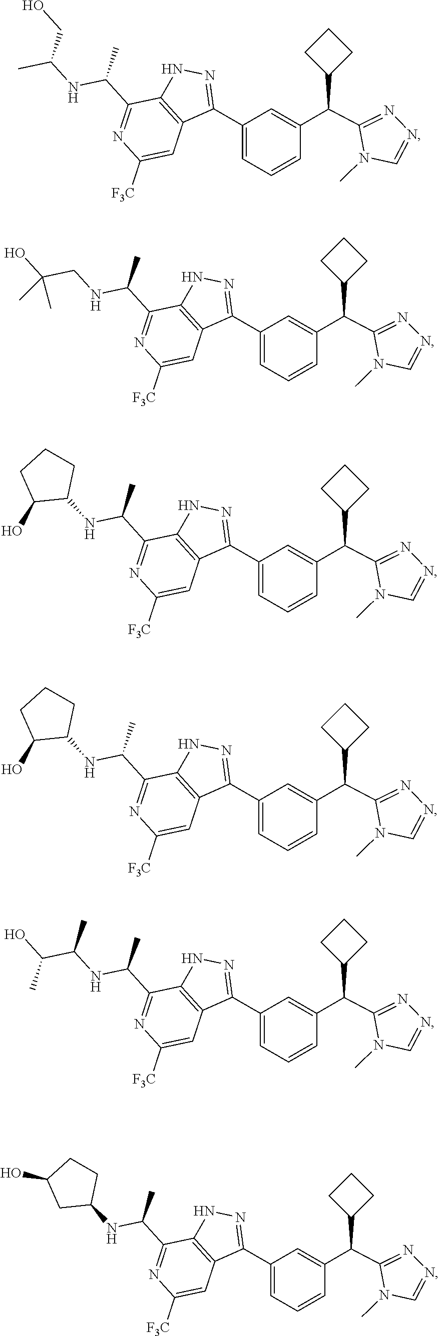

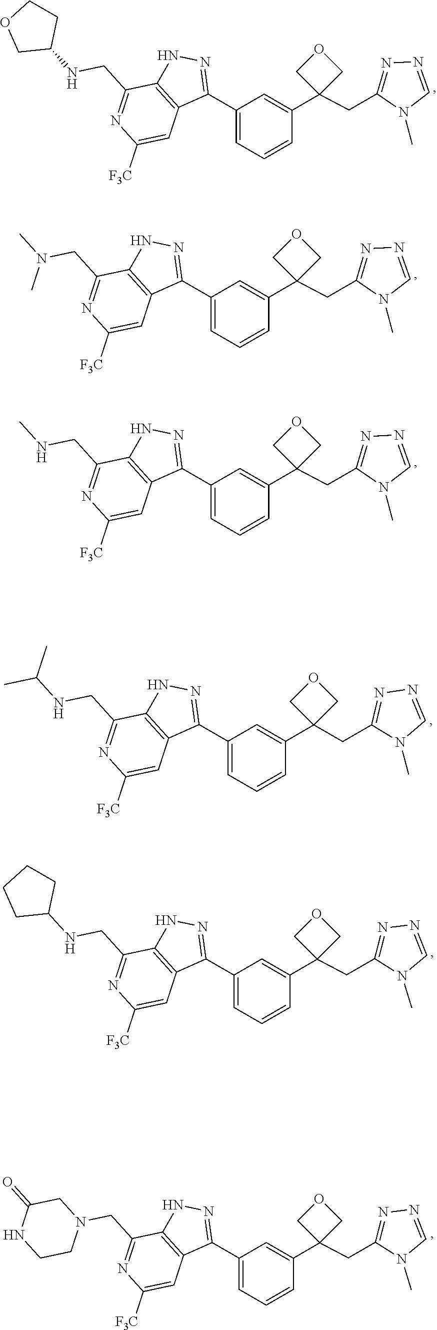 [SMILES: Cn1cnnc1CC1(c2cccc(-c3n[nH]c4c(CN[C@H]5CCOC5)nc(C(F)(F)F)cc34)c2)COC1 ; CN(C)Cc1nc(C(F)(F)F)cc2c(-c3cccc(C4(Cc5nncn5C)COC4)c3)n[nH]c12 ; CNCc1nc(C(F)(F)F)cc2c(-c3cccc(C4(Cc5nncn5C)COC4)c3)n[nH]c12 ; CC(C)NCc1nc(C(F)(F)F)cc2c(-c3cccc(C4(Cc5nncn5C)COC4)c3)n[nH]c12 ; Cn1cnnc1CC1(c2cccc(-c3n[nH]c4c(CNC5CCCC5)nc(C(F)(F)F)cc34)c2)COC1 ; Cn1cnnc1CC1(c2cccc(-c3n[nH]c4c(CN5CCNC(=O)C5)nc(C(F)(F)F)cc34)c2)COC1]

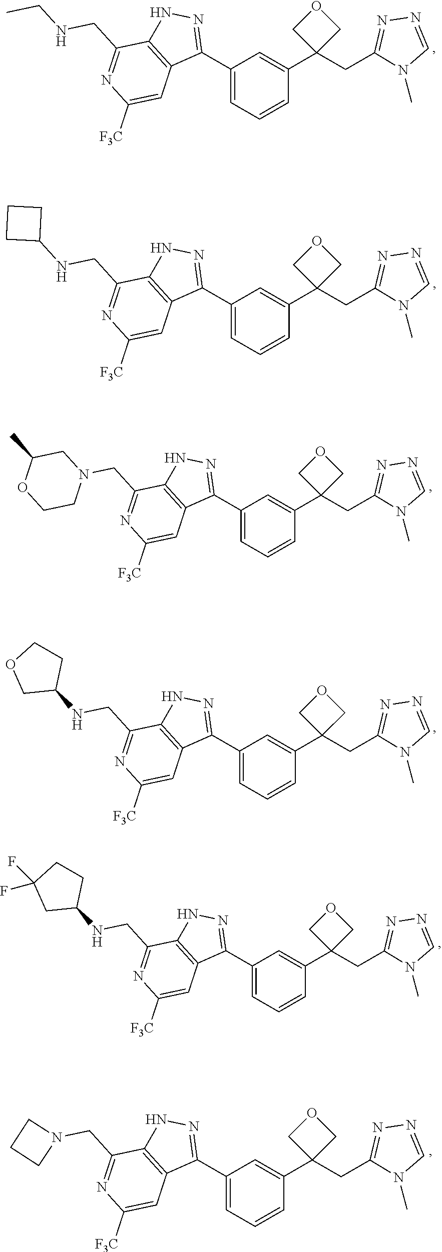 [SMILES: CCNCc1nc(C(F)(F)F)cc2c(-c3cccc(C4(Cc5nncn5C)COC4)c3)n[nH]c12 ; Cn1cnnc1CC1(c2cccc(-c3n[nH]c4c(CNC5CCC5)nc(C(F)(F)F)cc34)c2)COC1 ; C[C@H]1CN(Cc2nc(C(F)(F)F)cc3c(-c4cccc(C5(Cc6nncn6C)COC5)c4)n[nH]c23)CCO1 ; Cn1cnnc1CC1(c2cccc(-c3n[nH]c4c(CN[C@@H]5CCOC5)nc(C(F)(F)F)cc34)c2)COC1 ; Cn1cnnc1CC1(c2cccc(-c3n[nH]c4c(CN[C@@H]5CCC(F)(F)C5)nc(C(F)(F)F)cc34)c2)COC1 ; Cn1cnnc1CC1(c2cccc(-c3n[nH]c4c(CN5CCC5)nc(C(F)(F)F)cc34)c2)COC1]

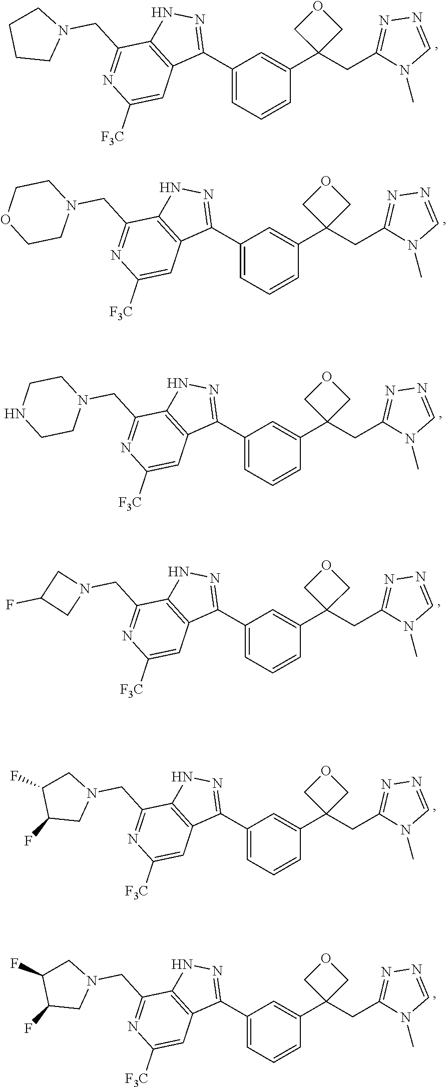 [SMILES: Cn1cnnc1CC1(c2cccc(-c3n[nH]c4c(CN5CCCC5)nc(C(F)(F)F)cc34)c2)COC1 ; Cn1cnnc1CC1(c2cccc(-c3n[nH]c4c(CN5CCOCC5)nc(C(F)(F)F)cc34)c2)COC1 ; Cn1cnnc1CC1(c2cccc(-c3n[nH]c4c(CN5CCNCC5)nc(C(F)(F)F)cc34)c2)COC1 ; Cn1cnnc1CC1(c2cccc(-c3n[nH]c4c(CN5CC(F)C5)nc(C(F)(F)F)cc34)c2)COC1 ; Cn1cnnc1CC1(c2cccc(-c3n[nH]c4c(CN5C[C@@H](F)[C@H](F)C5)nc(C(F)(F)F)cc34)c2)COC1 ; Cn1cnnc1CC1(c2cccc(-c3n[nH]c4c(CN5C[C@@H](F)[C@@H](F)C5)nc(C(F)(F)F)cc34)c2)COC1]

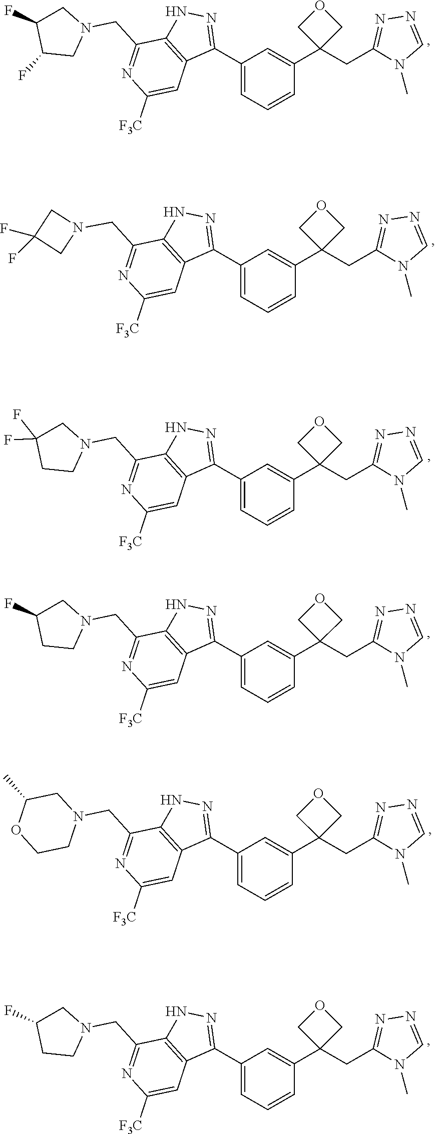 [SMILES: Cn1cnnc1CC1(c2cccc(-c3n[nH]c4c(CN5C[C@H](F)[C@@H](F)C5)nc(C(F)(F)F)cc34)c2)COC1 ; Cn1cnnc1CC1(c2cccc(-c3n[nH]c4c(CN5CC(F)(F)C5)nc(C(F)(F)F)cc34)c2)COC1 ; Cn1cnnc1CC1(c2cccc(-c3n[nH]c4c(CN5CCC(F)(F)C5)nc(C(F)(F)F)cc34)c2)COC1 ; Cn1cnnc1CC1(c2cccc(-c3n[nH]c4c(CN5CC[C@@H](F)C5)nc(C(F)(F)F)cc34)c2)COC1 ; C[C@@H]1CN(Cc2nc(C(F)(F)F)cc3c(-c4cccc(C5(Cc6nncn6C)COC5)c4)n[nH]c23)CCO1 ; Cn1cnnc1CC1(c2cccc(-c3n[nH]c4c(CN5CC[C@H](F)C5)nc(C(F)(F)F)cc34)c2)COC1]

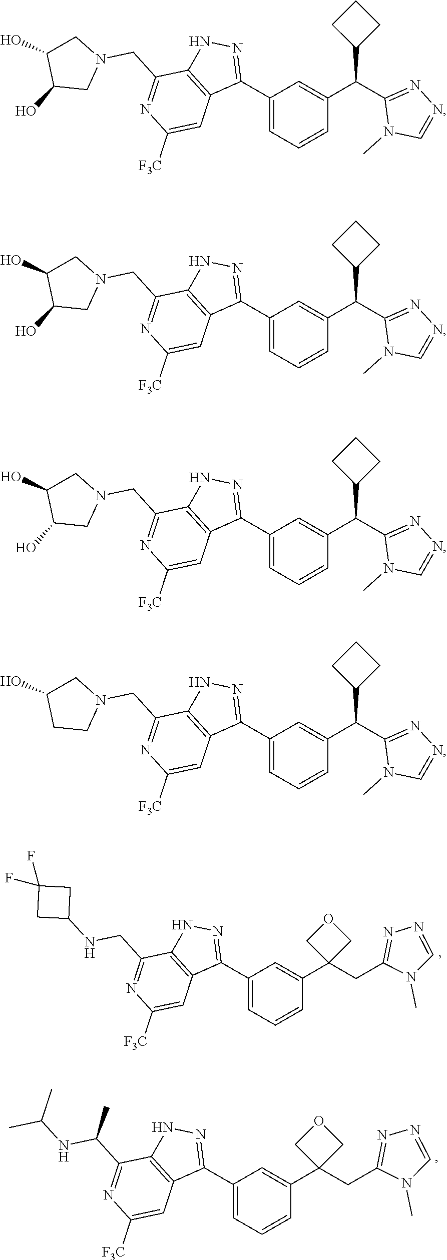 [SMILES: Cn1cnnc1[C@@H](c1cccc(-c2n[nH]c3c(CN4C[C@@H](O)[C@H](O)C4)nc(C(F)(F)F)cc23)c1)C1CCC1 ; Cn1cnnc1[C@@H](c1cccc(-c2n[nH]c3c(CN4C[C@@H](O)[C@@H](O)C4)nc(C(F)(F)F)cc23)c1)C1CCC1 ; Cn1cnnc1[C@@H](c1cccc(-c2n[nH]c3c(CN4C[C@H](O)[C@@H](O)C4)nc(C(F)(F)F)cc23)c1)C1CCC1 ; Cn1cnnc1[C@@H](c1cccc(-c2n[nH]c3c(CN4CC[C@H](O)C4)nc(C(F)(F)F)cc23)c1)C1CCC1 ; Cn1cnnc1CC1(c2cccc(-c3n[nH]c4c(CNC5CC(F)(F)C5)nc(C(F)(F)F)cc34)c2)COC1 ; CC(C)N[C@@H](C)c1nc(C(F)(F)F)cc2c(-c3cccc(C4(Cc5nncn5C)COC4)c3)n[nH]c12]

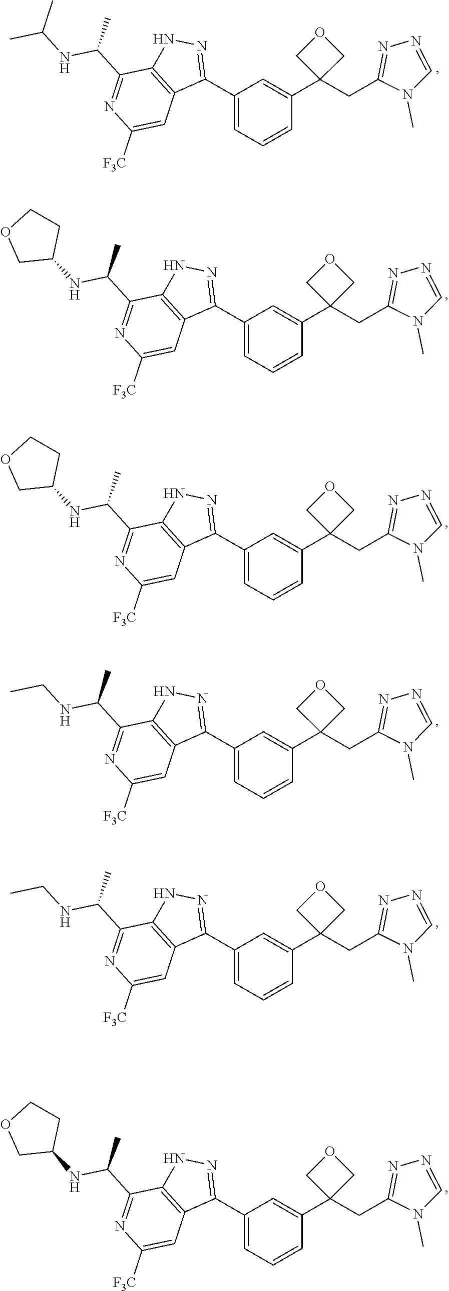 [SMILES: CC(C)N[C@H](C)c1nc(C(F)(F)F)cc2c(-c3cccc(C4(Cc5nncn5C)COC4)c3)n[nH]c12 ; C[C@H](N[C@H]1CCOC1)c1nc(C(F)(F)F)cc2c(-c3cccc(C4(Cc5nncn5C)COC4)c3)n[nH]c12 ; C[C@@H](N[C@H]1CCOC1)c1nc(C(F)(F)F)cc2c(-c3cccc(C4(Cc5nncn5C)COC4)c3)n[nH]c12 ; CCN[C@@H](C)c1nc(C(F)(F)F)cc2c(-c3cccc(C4(Cc5nncn5C)COC4)c3)n[nH]c12 ; CCN[C@H](C)c1nc(C(F)(F)F)cc2c(-c3cccc(C4(Cc5nncn5C)COC4)c3)n[nH]c12 ; C[C@H](N[C@@H]1CCOC1)c1nc(C(F)(F)F)cc2c(-c3cccc(C4(Cc5nncn5C)COC4)c3)n[nH]c12]

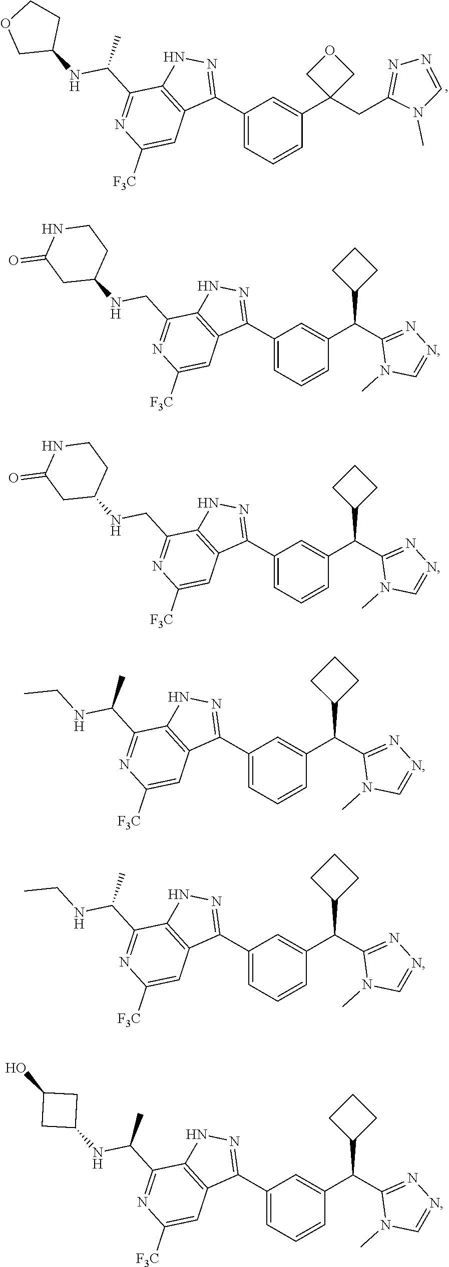

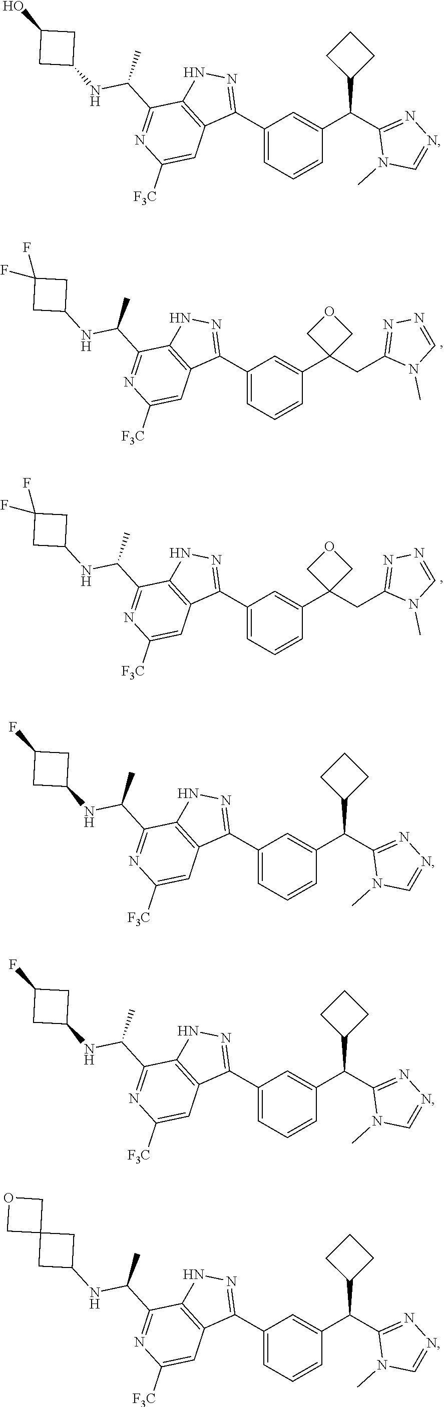

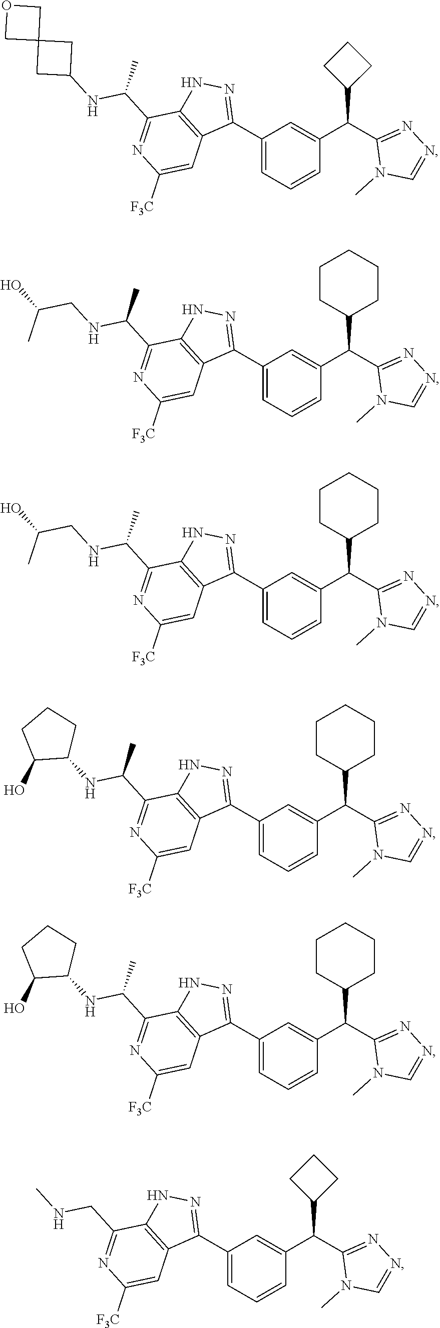

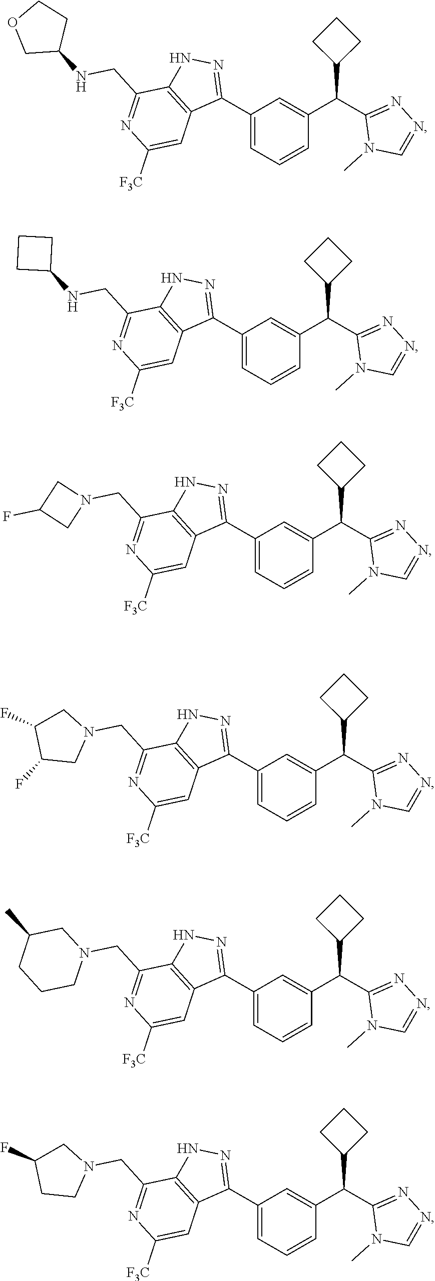

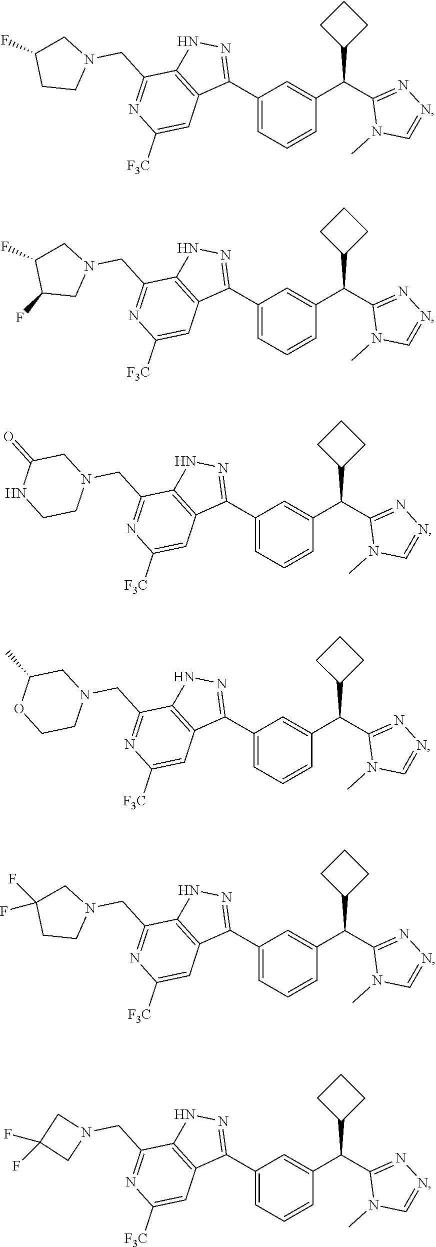 [SMILES: Cn1cnnc1[C@@H](c1cccc(-c2n[nH]c3c(CN4CC[C@H](F)C4)nc(C(F)(F)F)cc23)c1)C1CCC1 ; Cn1cnnc1[C@@H](c1cccc(-c2n[nH]c3c(CN4C[C@@H](F)[C@H](F)C4)nc(C(F)(F)F)cc23)c1)C1CCC1 ; Cn1cnnc1[C@@H](c1cccc(-c2n[nH]c3c(CN4CCNC(=O)C4)nc(C(F)(F)F)cc23)c1)C1CCC1 ; C[C@@H]1CN(Cc2nc(C(F)(F)F)cc3c(-c4cccc([C@H](c5nncn5C)C5CCC5)c4)n[nH]c23)CCO1 ; Cn1cnnc1[C@@H](c1cccc(-c2n[nH]c3c(CN4CCC(F)(F)C4)nc(C(F)(F)F)cc23)c1)C1CCC1 ; Cn1cnnc1[C@@H](c1cccc(-c2n[nH]c3c(CN4CC(F)(F)C4)nc(C(F)(F)F)cc23)c1)C1CCC1]

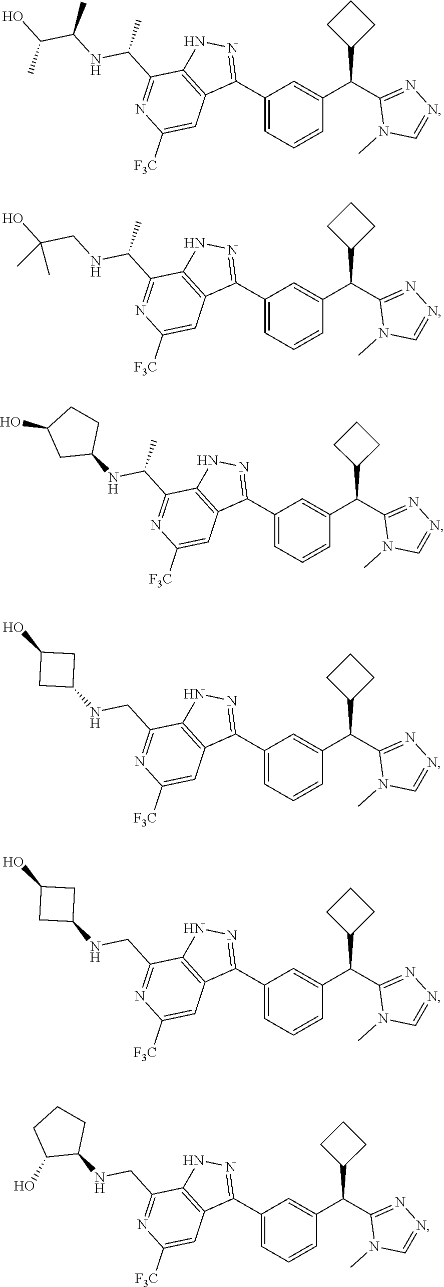

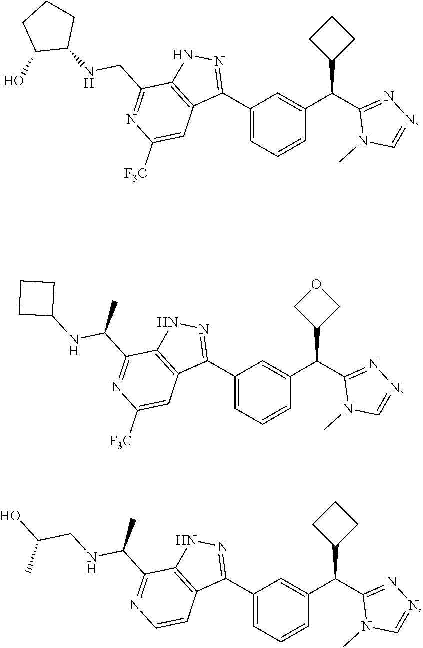 [SMILES: Cn1cnnc1[C@@H](c1cccc(-c2n[nH]c3c(CN[C@H]4CCC[C@H]4O)nc(C(F)(F)F)cc23)c1)C1CCC1 ; C[C@H](NC1CCC1)c1nc(C(F)(F)F)cc2c(-c3cccc([C@H](c4nncn4C)C4COC4)c3)n[nH]c12 ; C[C@H](O)CN[C@@H](C)c1nccc2c(-c3cccc([C@H](c4nncn4C)C4CCC4)c3)n[nH]c12]

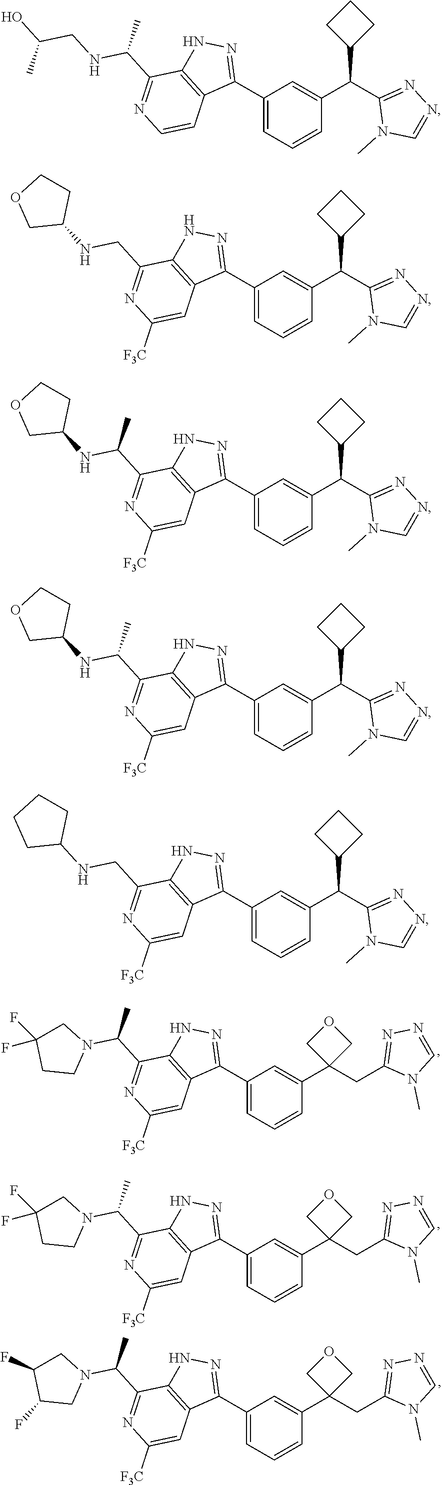

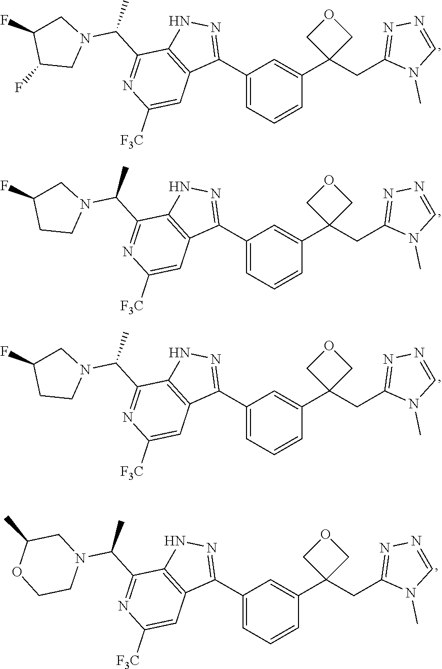

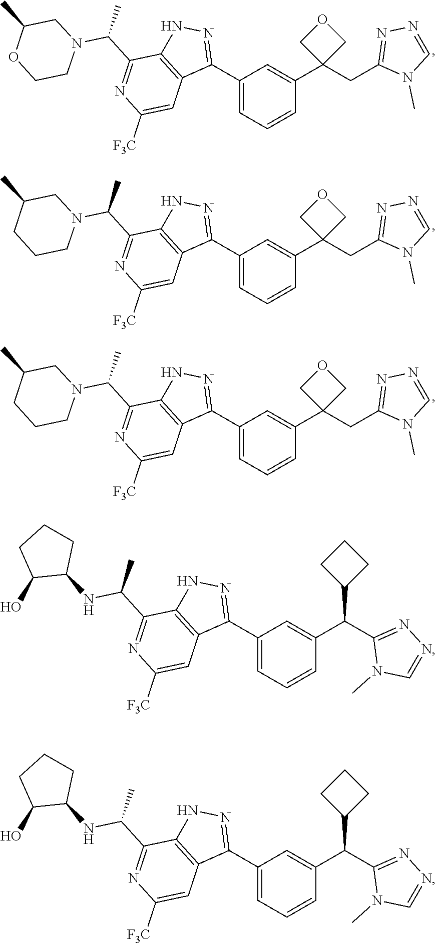

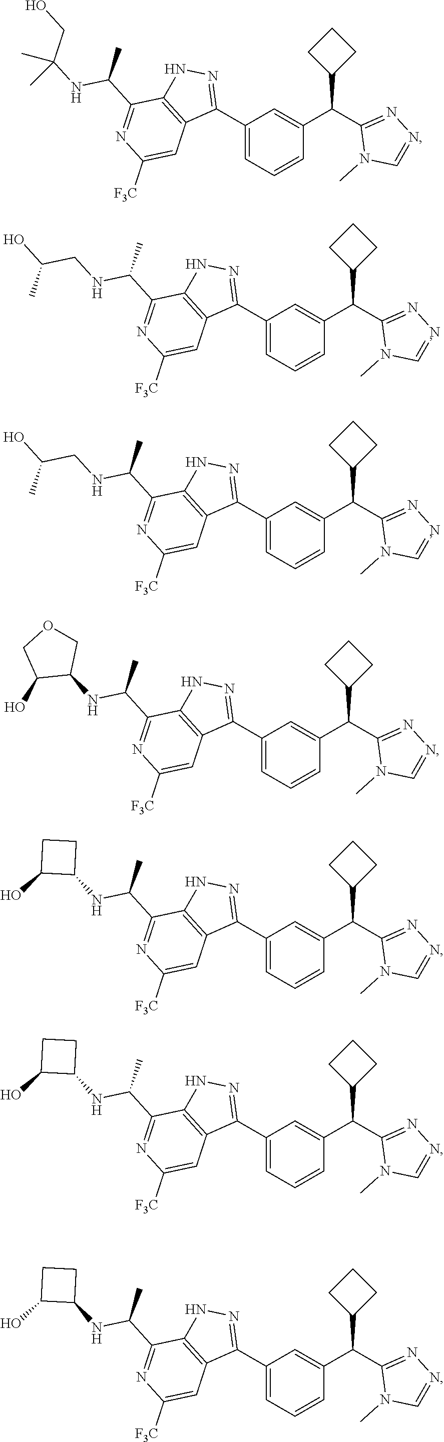

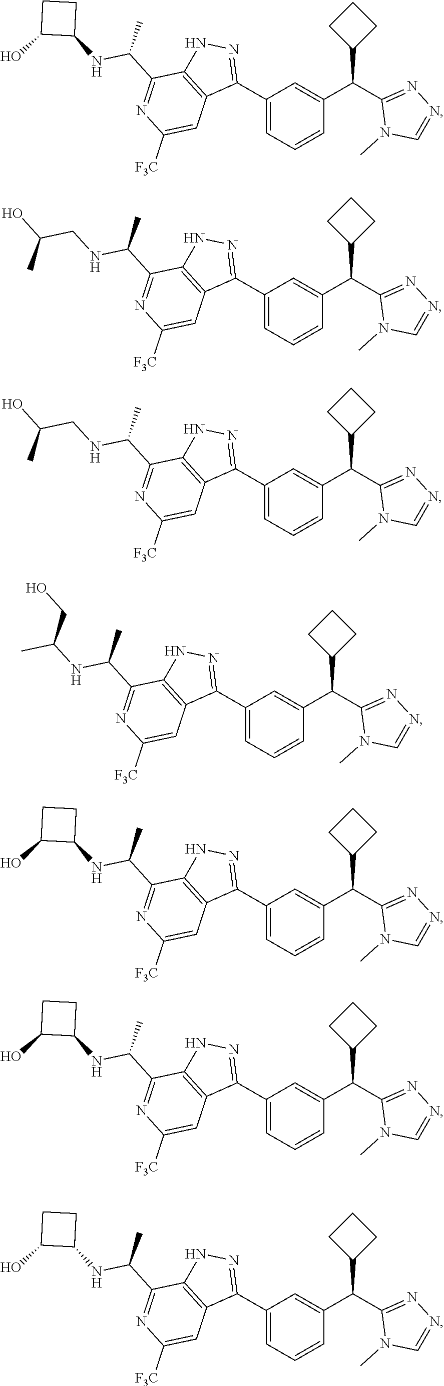

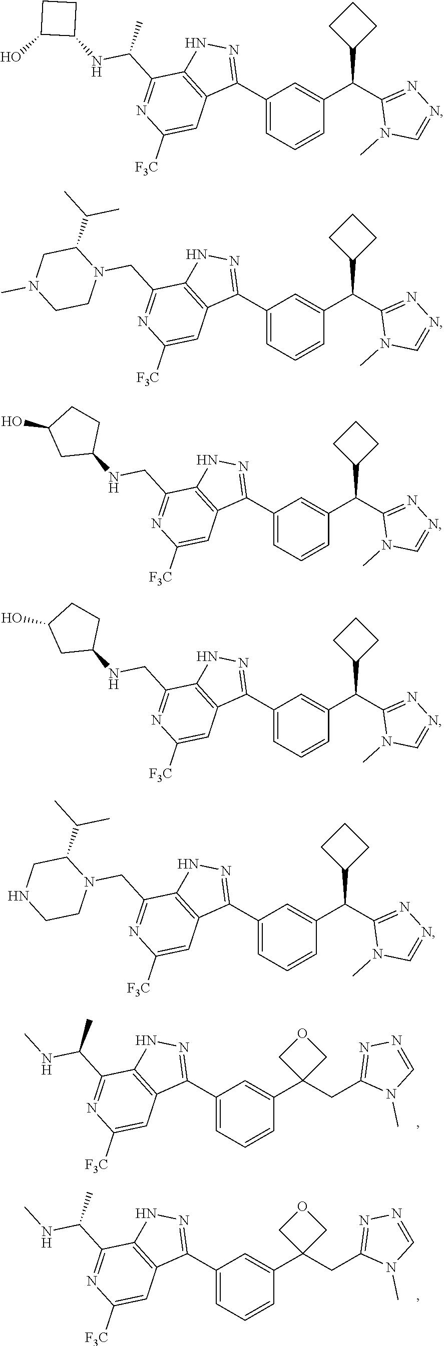

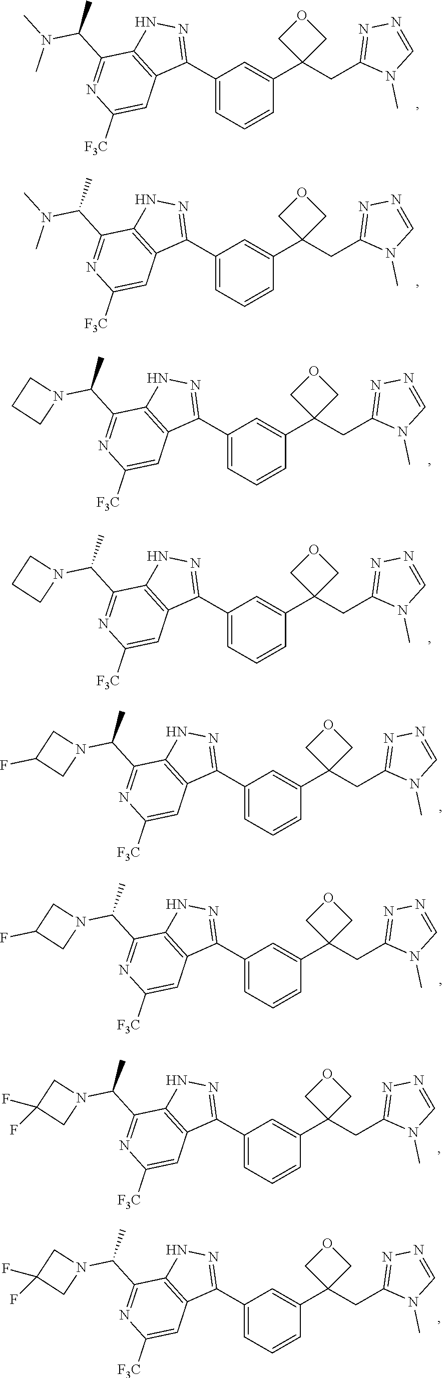

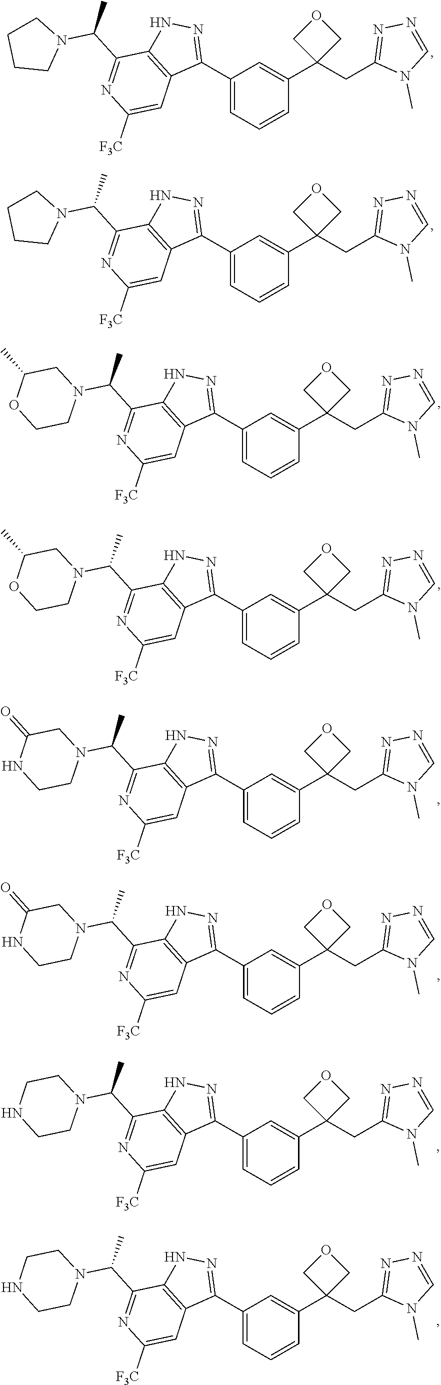

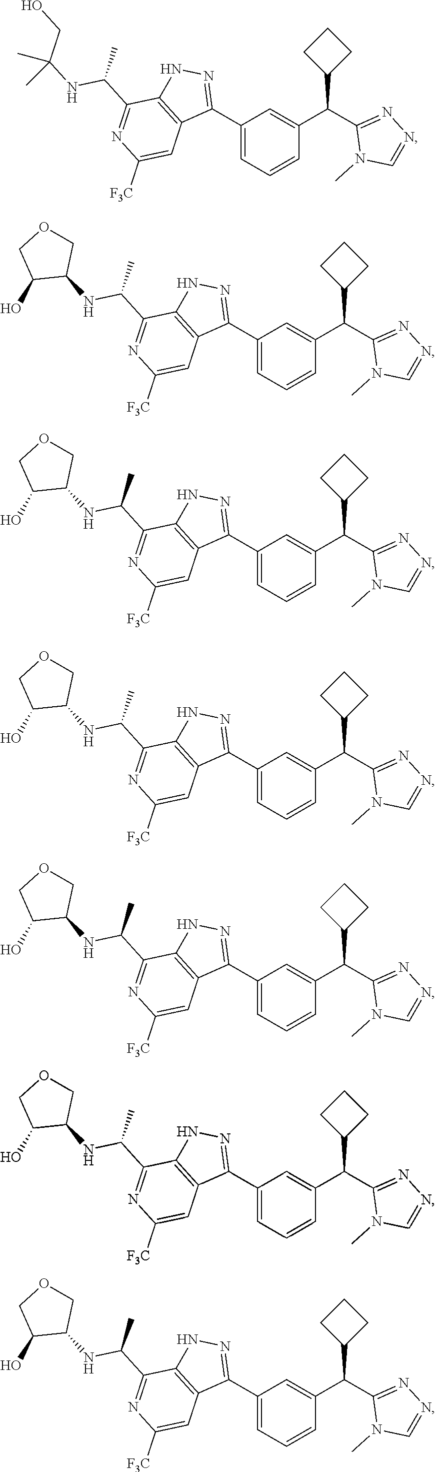

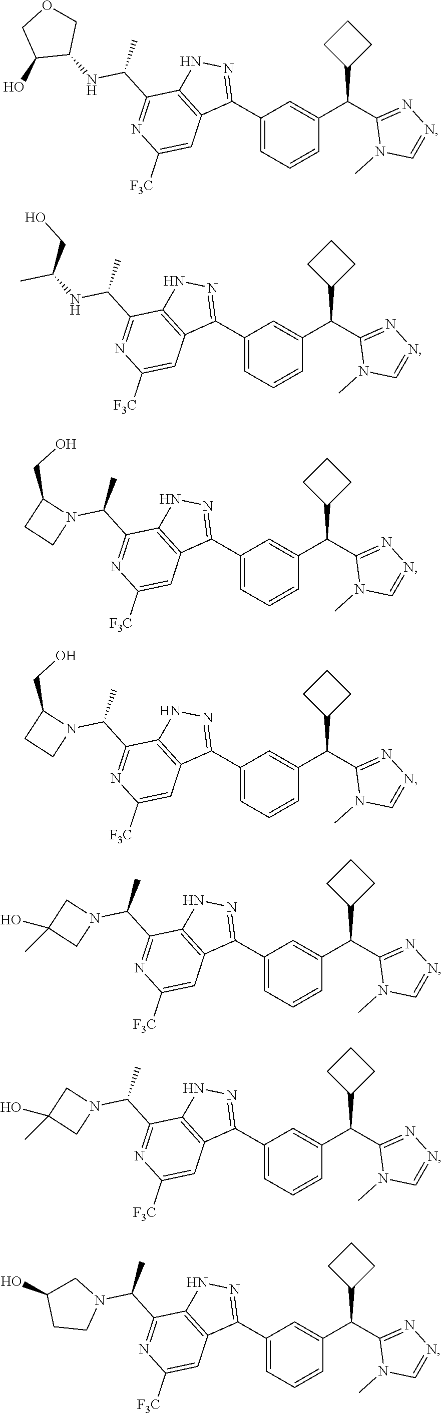

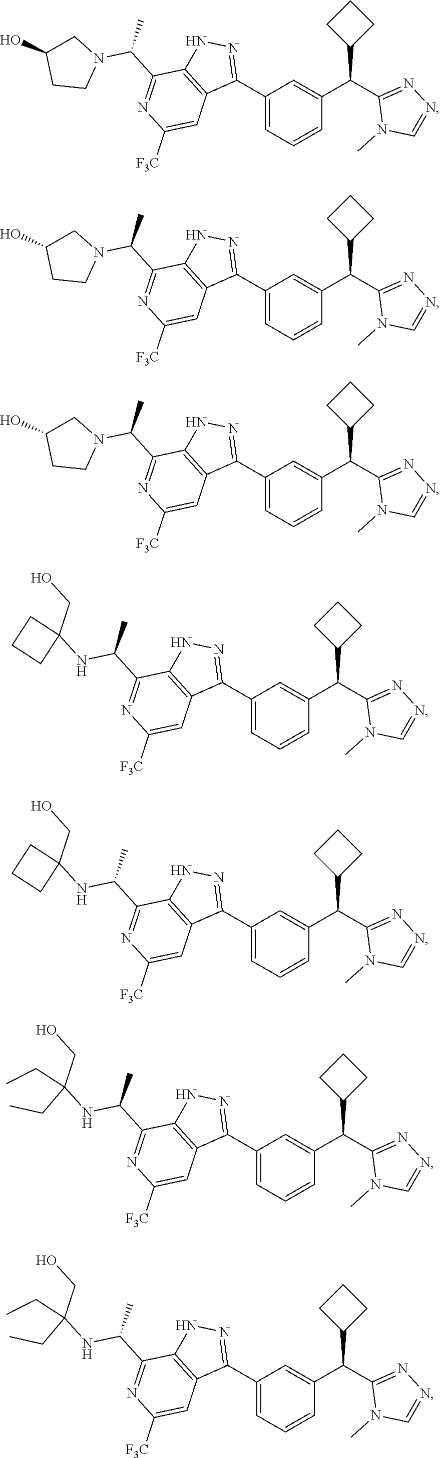

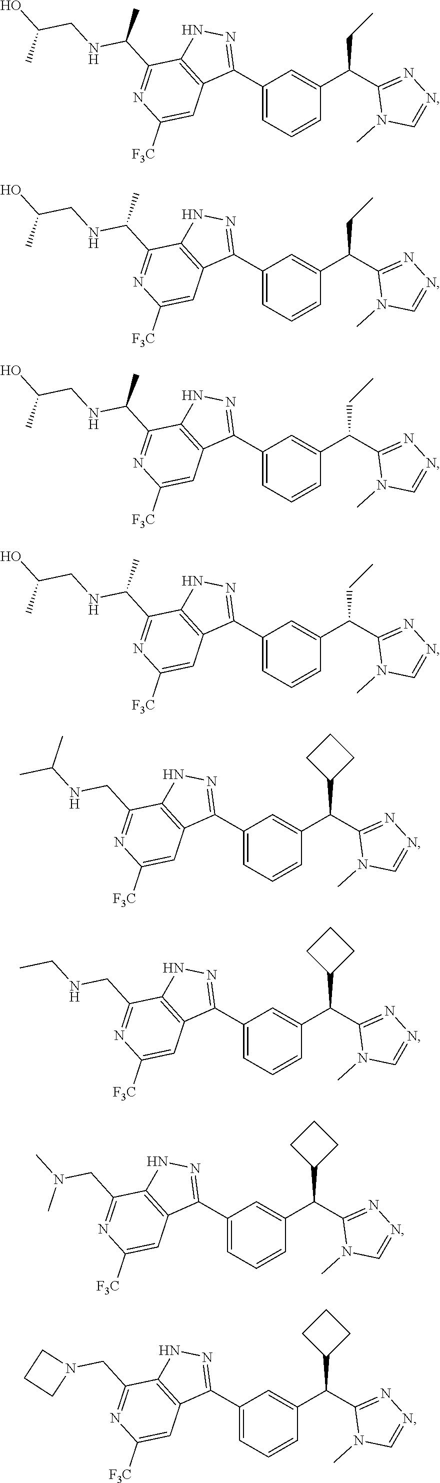

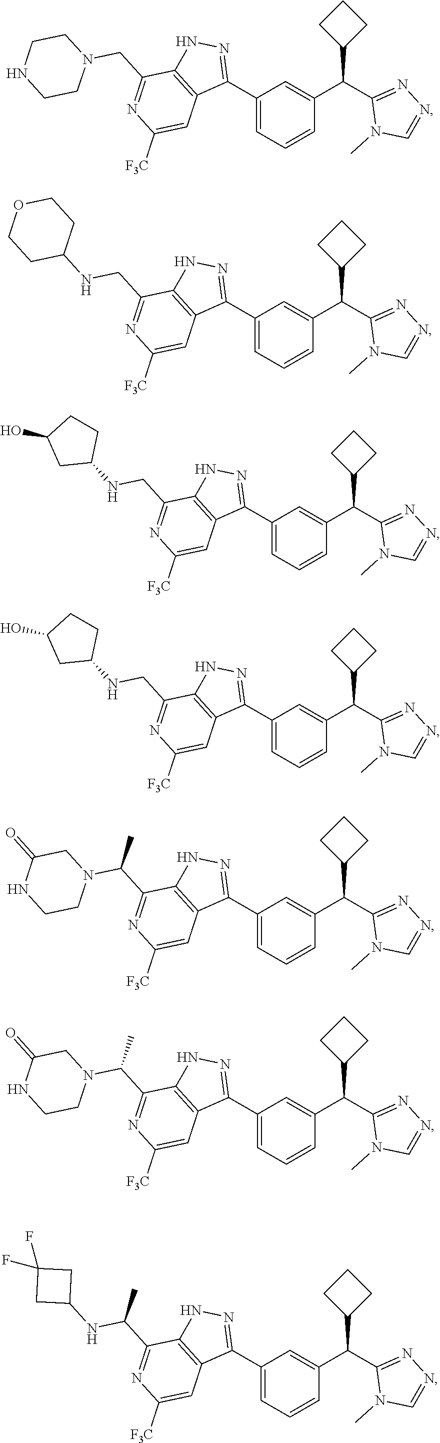

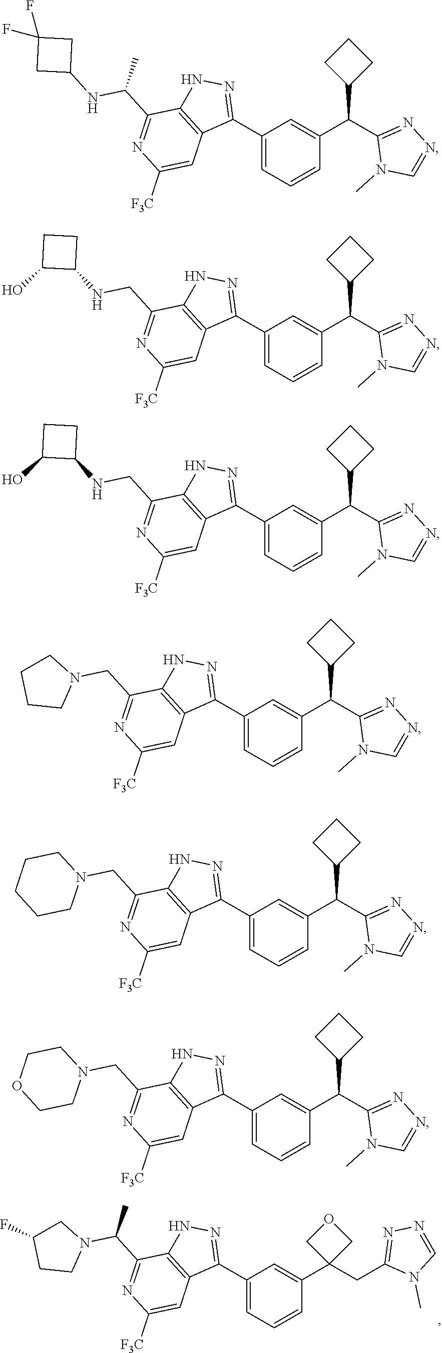

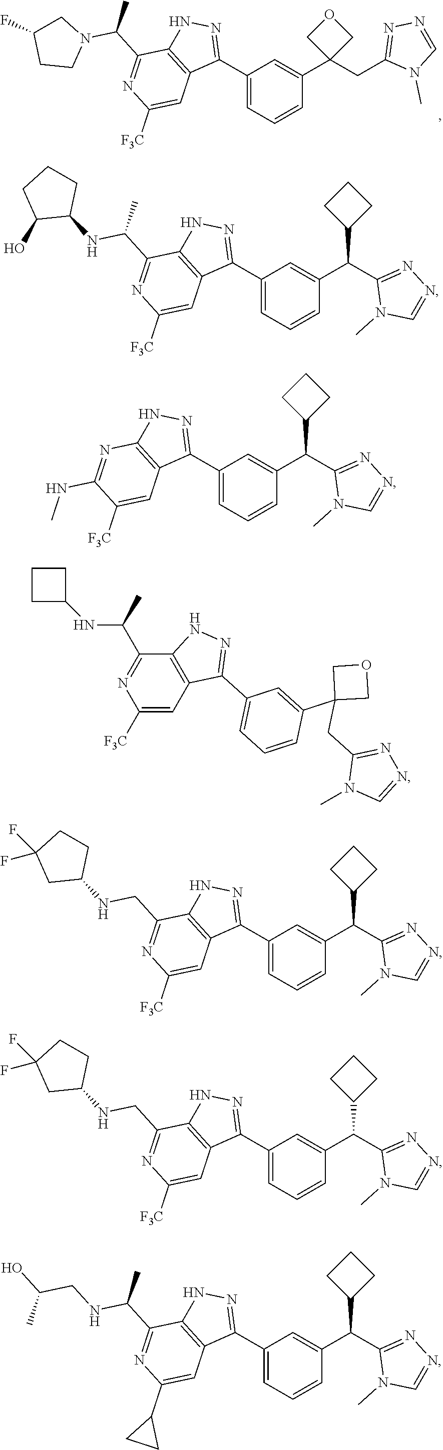

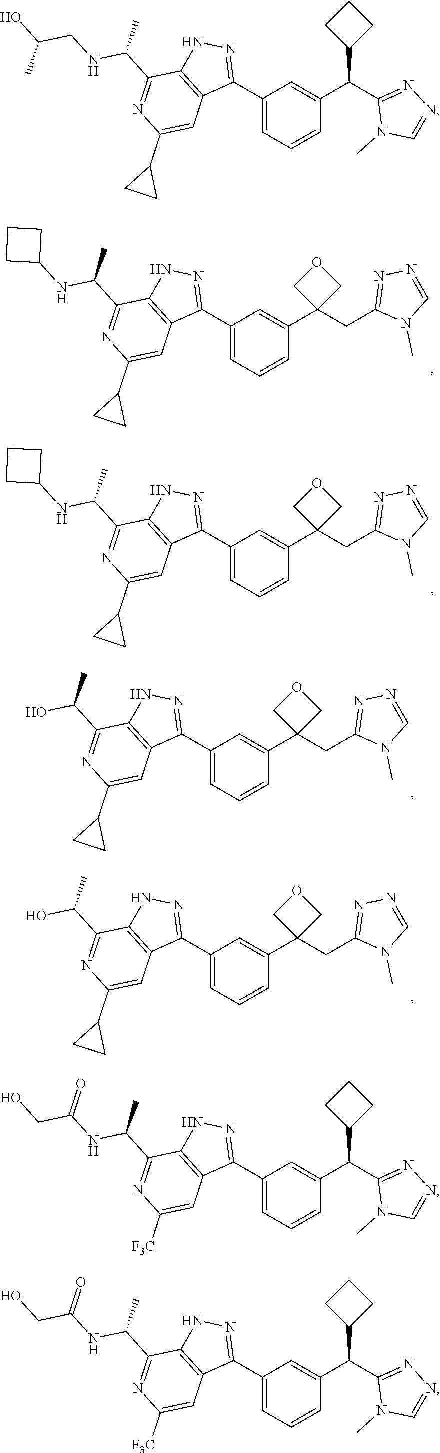

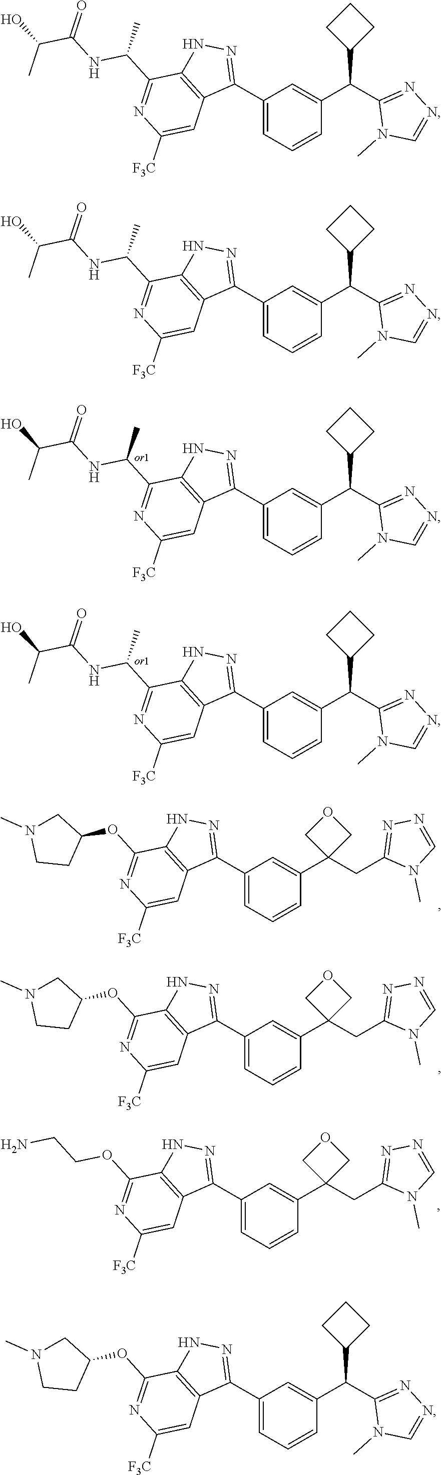

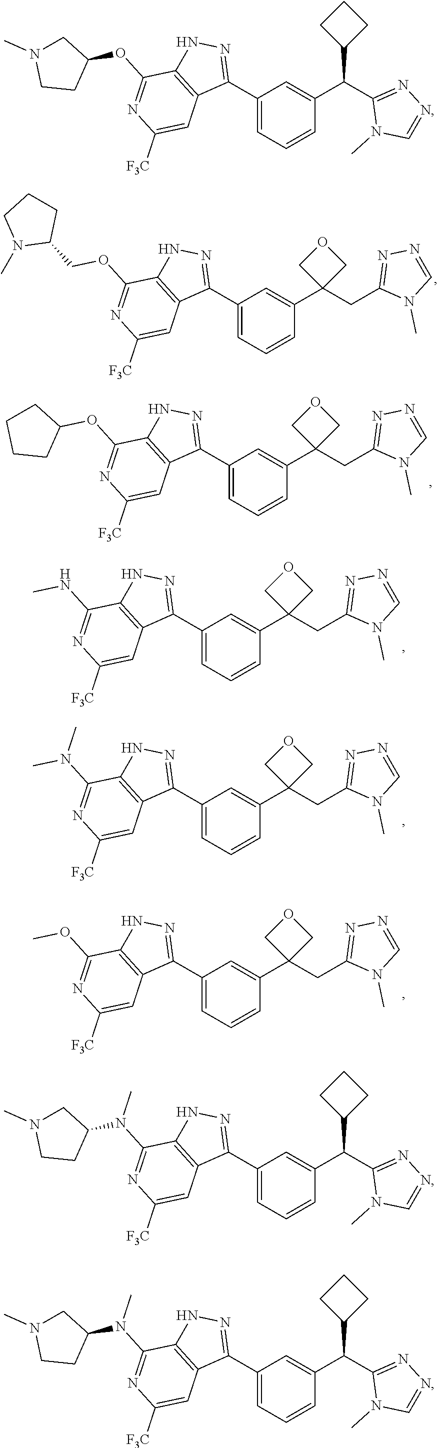

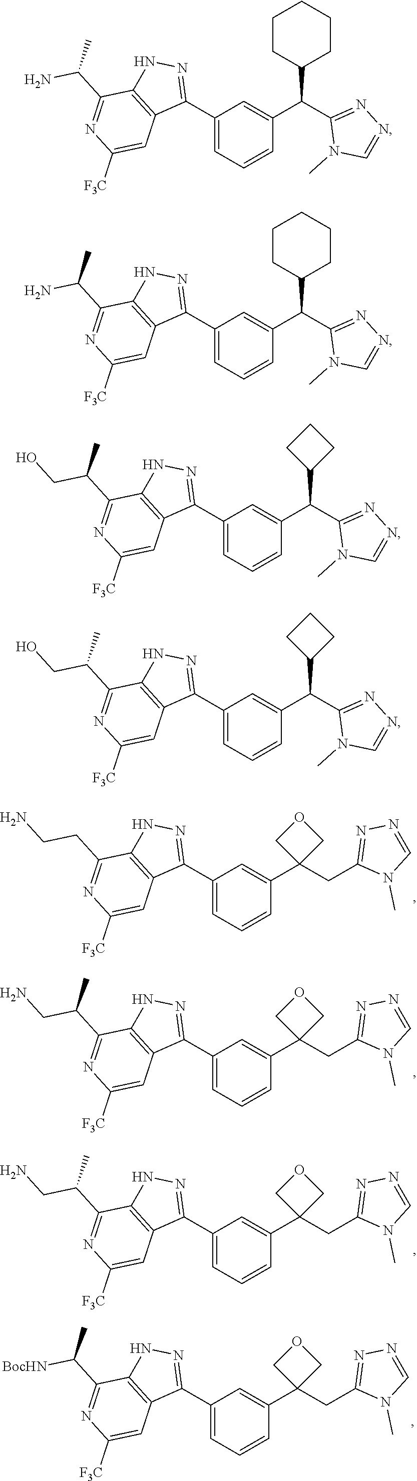

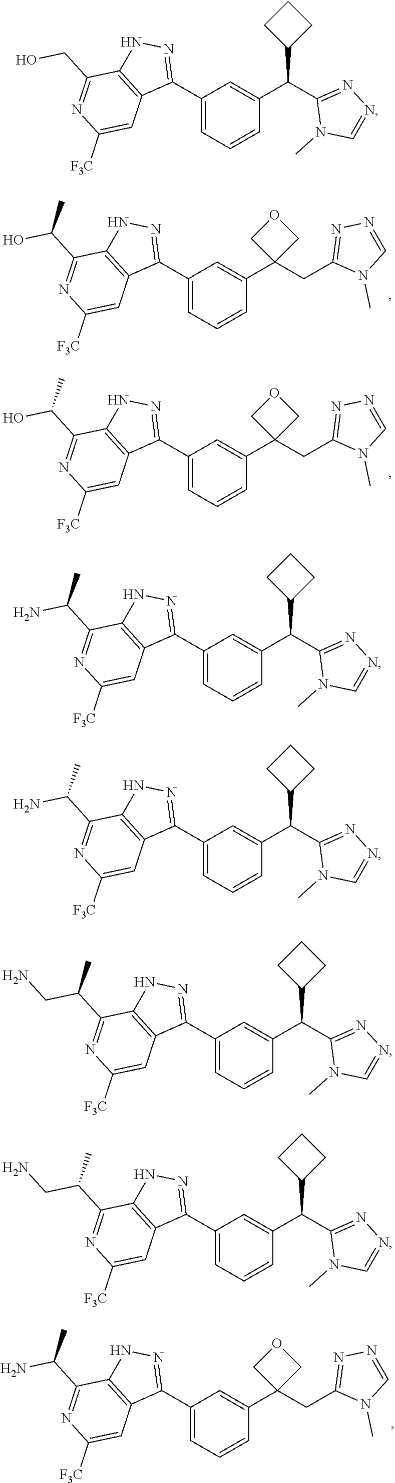

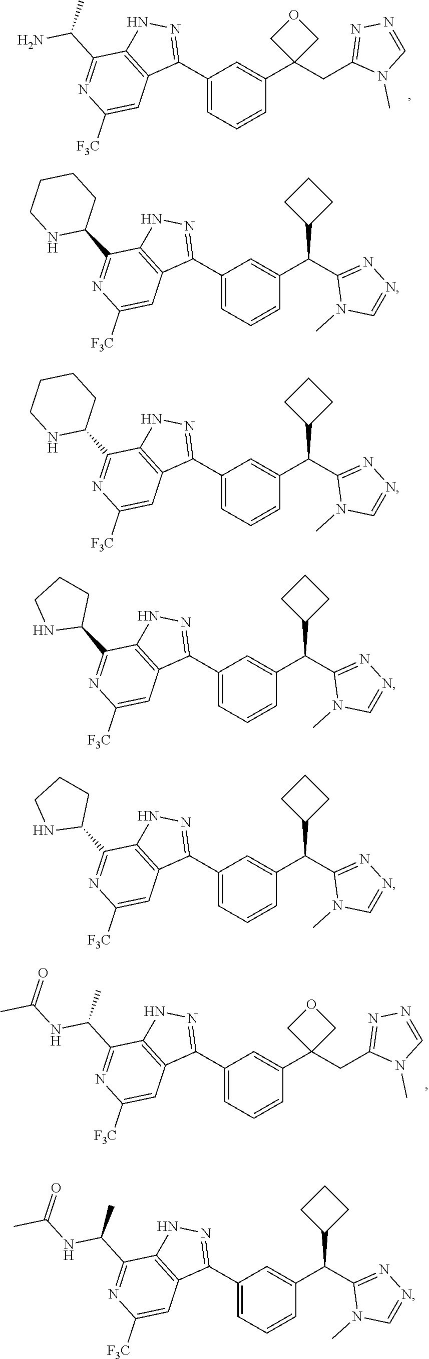

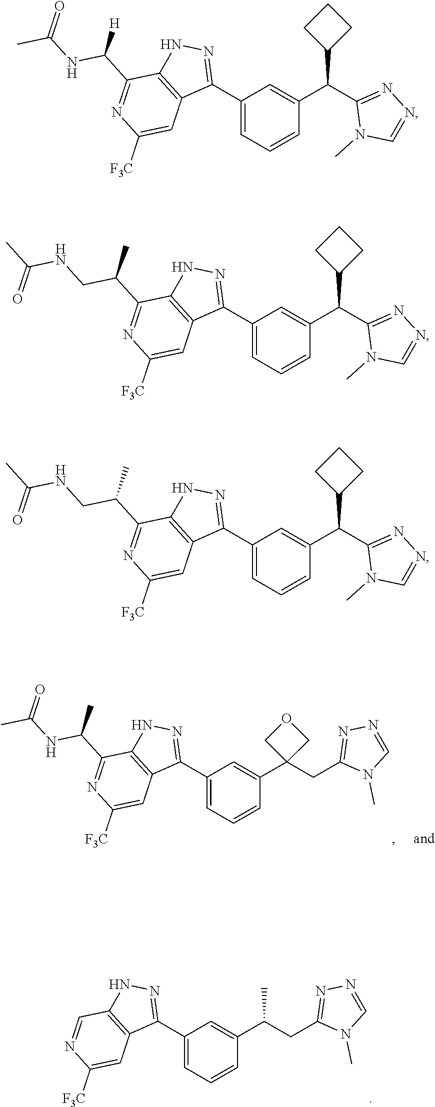

E49 The compound of any one of embodiments 1-48, or a pharmaceutically acceptable salt thereof, wherein the compound is selected from the group consisting of:

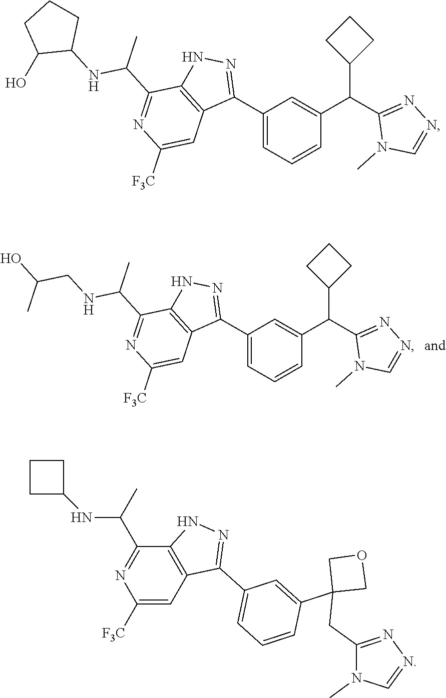 [SMILES: CC(NC1CCCC1O)c1nc(C(F)(F)F)cc2c(-c3cccc(C(c4nncn4C)C4CCC4)c3)n[nH]c12 ; CC(O)CNC(C)c1nc(C(F)(F)F)cc2c(-c3cccc(C(c4nncn4C)C4CCC4)c3)n[nH]c12 ; CC(NC1CCC1)c1nc(C(F)(F)F)cc2c(-c3cccc(C4(Cc5nncn5C)COC4)c3)n[nH]c12]

E50 A compound which is selected from the group consisting of:

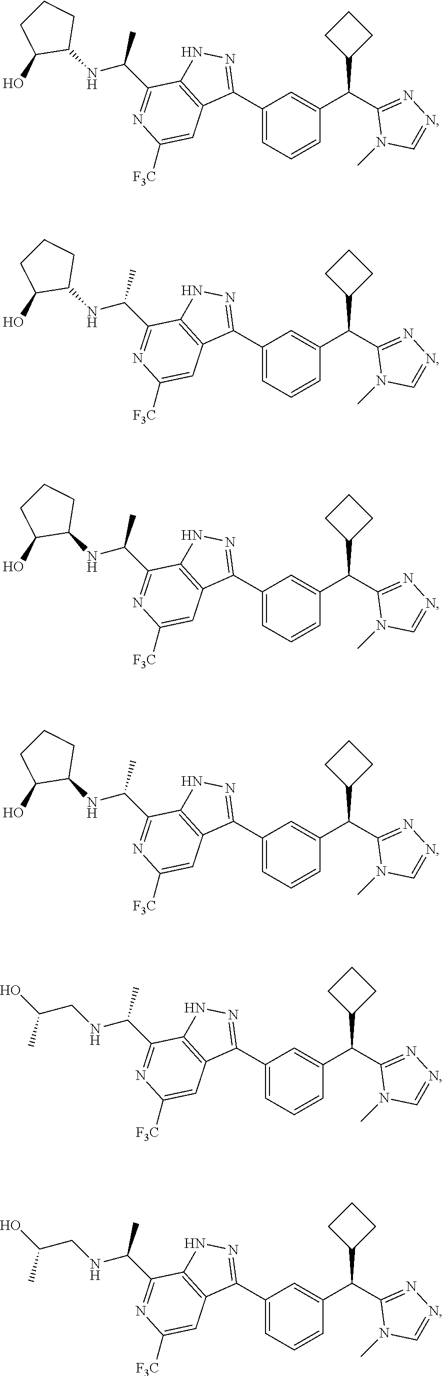

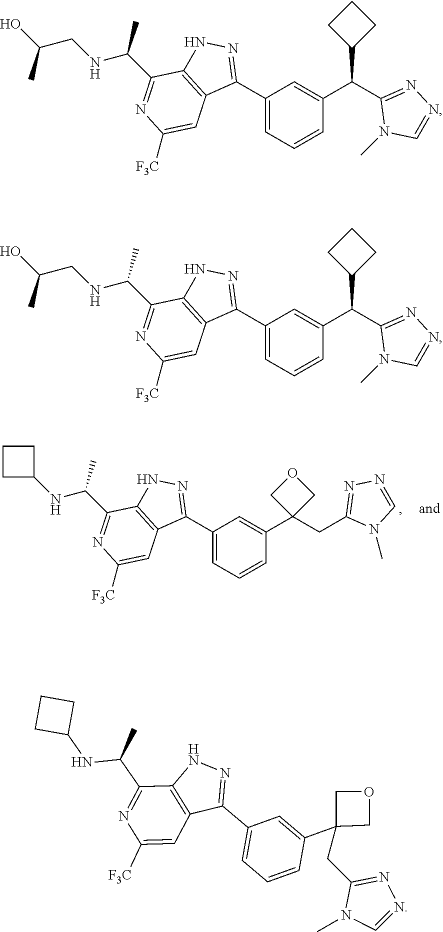 [SMILES: C[C@H](NC[C@@H](C)O)c1nc(C(F)(F)F)cc2c(-c3cccc([C@H](c4nncn4C)C4CCC4)c3)n[nH]c12 ; C[C@@H](O)CN[C@H](C)c1nc(C(F)(F)F)cc2c(-c3cccc([C@H](c4nncn4C)C4CCC4)c3)n[nH]c12 ; C[C@@H](NC1CCC1)c1nc(C(F)(F)F)cc2c(-c3cccc(C4(Cc5nncn5C)COC4)c3)n[nH]c12 ; C[C@H](NC1CCC1)c1nc(C(F)(F)F)cc2c(-c3cccc(C4(Cc5nncn5C)COC4)c3)n[nH]c12]

E51 A pharmaceutical composition comprising the compound according to any of embodiments E1 to E50, or a pharmaceutically acceptable salt thereof, and at least one pharmaceutically acceptable excipient.
E52 A method for treating cancer in a subject in need thereof, comprising administering to the subject a therapeutically effective amount of the compound of any of embodiments E1 to E50, or a pharmaceutically acceptable salt thereof.
E53 A method for treating cancer in a subject in need thereof, comprising administering to the subject a therapeutically effective amount of a compound of any one of embodiments E1 to E50, or a pharmaceutically acceptable salt thereof, as a single agent.
E54 A method for treating cancer in a subject in need thereof, comprising administering to the subject a therapeutically effective amount of a compound of any one of embodiments E1 to E50, or a pharmaceutically acceptable salt thereof, and further comprising administering a therapeutically effective amount of an additional anticancer therapeutic agent.
E55 A compound according to any of embodiments E1 to E50 for use as a medicament.
E56 A compound according to any of embodiments E1 to E50 for use in the treatment of cancer in a subject.
E57 Use of a compound according to any of embodiments E1 to E50 for the manufacture of a medicament for the treatment of cancer in a subject.
E58 A method for the treatment of a disorder mediated by inhibition of CBL-B in a subject, comprising administering to the subject in need thereof a compound of any one of embodiments E1 to E50, or a pharmaceutically acceptable salt thereof, in an amount that is effective for treating the disorder.
E59 A pharmaceutical combination comprising a compound of any one of embodiments E1 to E50 or a pharmaceutically acceptable salt thereof, and at least one additional therapeutic agent or a pharmaceutically acceptable salt thereof.
E60 A pharmaceutical composition comprising the pharmaceutical combination of embodiment E59 and at least one excipient.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

Furthermore, each of the embodiments described herein envisions within its scope pharmaceutically acceptable salts of the compounds described herein.

### Definitions

Unless otherwise defined herein, scientific and technical terms used in connection with the present disclosure have the meanings that are commonly understood by those of ordinary skill in the art.

The disclosure described herein suitably may be practiced in the absence of any element(s) not specifically disclosed herein.

“Compounds of the disclosure” include compounds of any of the formulae described herein, or a pharmaceutically acceptable salt thereof. One of ordinary skill in the art will appreciate that compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the disclosure include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5%±10%, i.e., it may vary between 4.5 mg and 5.5 mg.

A “bond” refers to a covalent linkage between two atoms, or two moieties when the atoms joined by the bond are considered to be part of larger substructure. In one aspect, when a group described herein is a bond, the referenced group is absent thereby allowing a bond to be formed between the remaining identified groups

“Halogen” or “halo” refers to fluoro, chloro, bromo and iodo (F, Cl, Br, I).

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein.

Alkyl groups described herein as optionally substituted may be substituted by one or more substituent groups, as further defined by the claims, which substituent groups are selected independently unless otherwise indicated. The total number of substituent groups may equal the total number of hydrogen atoms on the alkyl moiety, to the extent such substitution makes chemical sense. Optionally substituted alkyl groups typically contain from 1 to 6 optional substituents, sometimes 1 to 5 optional substituents, 1 to 4 optional substituents, or preferably 1 to 3 optional substituents.

In some instances, substituted alkyl groups are specifically named by reference to the substituent group. For example, “haloalkyl” refers to an alkyl group having the specified number of carbon atoms that is substituted by one or more halo substituents, up to the available valence number. Typically, haloalkyl groups contain 1-6 carbon atoms, 1-5 carbon atoms, 1-4 carbon atoms, 1-3 carbon atoms, or 1-2 carbon atoms (i.e., “C₁-C₅ haloalkyl”, “C₁-C₄ haloalkyl”, “C₁-C₃ haloalkyl”, or “C₁-C₂ haloalkyl”) and with 1, 2, 3, 4, 5 or more halo atoms. More specifically, fluorinated alkyl groups may be specifically referred to as “fluoroalkyl.”

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkylene” refers to a divalent hydrocarbyl group having the specified number of carbon atoms which can link two other groups together. Sometimes it refers to a group —(CH₂)ₙ— where n is 1-3 (also refers to as “C₁-C₃ alkylene”), or n is 1-2 (i.e., “C₁-C₂ alkylene”), or n is 1 (e.g., methylene). Where specified, an alkylene may also be substituted by other groups and may include one or more degrees of unsaturation (i.e., an alkenylene or alkynlene moiety) or rings. The open valences of an alkylene need not be at opposite ends of the chain. Thus, branched alkylene groups such as —CH(Me)- and —C(Me)₂- are also included within the scope of the term “alkylenes”, as are cyclic groups such as cyclopropan-1,1-diyl and unsaturated groups such as ethylene (—CH═CH—) or propylene (—CH₂—CH═CH—). Where an alkylene group is described as optionally substituted, the substituents include those described herein.

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 4 carbon atoms (“C₁-C₄ alkoxy”), 1 to 3 carbon atoms (“C₁-C₃ alkoxy”), 1 to 2 carbon atoms (“C₁-C₂ alkoxy”), or 1 carbon atom (“methoxy”). Alkoxy groups include, but are not limited to, methoxy, ethoxy, n-propoxy, isobutoxy, and the like. Alkoxy groups may be optionally substituted, unsubstituted or substituted, as further defined herein.

“Cycloalkyl” refers to a fully saturated hydrocarbon ring system that has the specified number of carbon atoms, which may be a monocyclic, bridged or fused bicyclic, spirocyclic or polycyclic ring system that is connected to the base molecule through a carbon atom of the cycloalkyl ring. Cycloalkyl groups may contain, but are not limited to, 3 to 10 carbon atoms (“C₃-C₁₀ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

Illustrative examples of spirocyclic cycloalkyl rings include, but are not limited to a monovalent radical of:

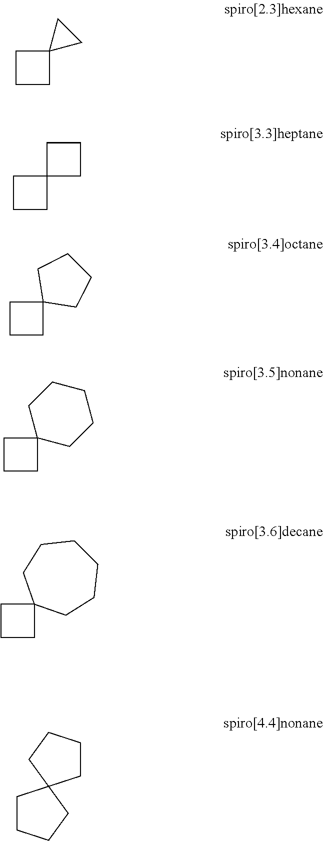

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms.

Heterocycloalkyl rings may be optionally substituted, unsubstituted or substituted, as further defined herein. Such substituents may be present on the heterocyclic ring attached to the base molecule, or on a spirocyclic, bridged or fused ring attached thereto.

Heterocycloalkyl rings may include, but are not limited to, 4-8 membered heterocycloalkyl groups, for example 4-7, or 4-6 membered heterocycloalkyl groups, in accordance with the definition herein.

Illustrative examples of heterocycloalkyl rings include, but are not limited to a monovalent radical of:

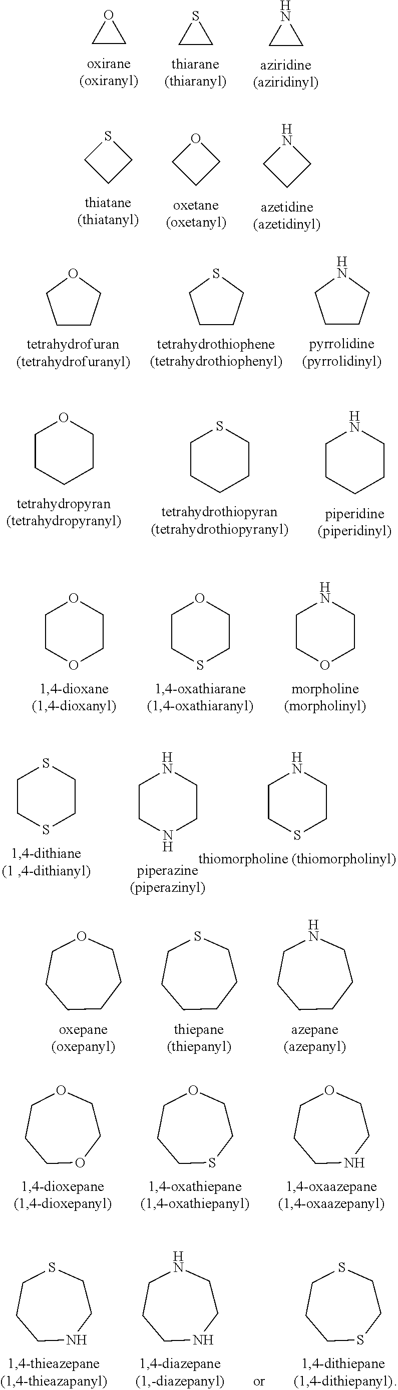

Illustrative examples of bridged, fused and spiro heterocycloalkyl rings include, but are not limited to:

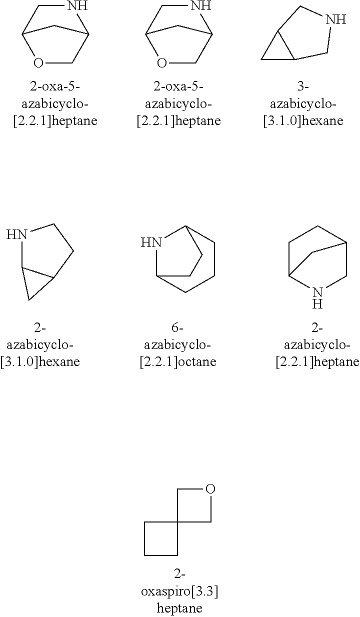

“Hydroxy,” as used herein, refers to OH.

“Hydroxyalkyl” refers to a hydroxy group, as defined above, attached to the parent molecular moiety through an alkyl group. Hydroxyalkyl groups may contain, but are not limited to, 1 to 6 carbon atoms (“hydroxy(C₁-C₆ alkyl)”), 1 to 3 carbon atoms (“hydroxy(C₁-C₃ alkyl)”), and 1 to 2 carbon atoms (“hydroxy(C₁-C₂ alkyl)”).

“Amino” refers to a group —NH₂, which is unsubstituted. Where the amino is described as substituted or optionally substituted, the term includes groups of the form —NR^{x}R^{y}, where each of R^{x} and R^{y} is defined as further described herein. For example, “alkylamino” refers to a group —NR^{x}R^{y}, wherein one of R^{x} and R^{y} is an alkyl moiety and the other is H, and “dialkylamino” refers to —NR^{x}R^{y} wherein both of R^{x} and R^{y} are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two.

When substituted, the substituents of an “optionally substituted” group may include, but are not limited to, halogen, —OH, C₁-C₆ alkyl, C₁-C₃ alkoxy, —CN, oxo, —COOR^{x}, —OC(O)R^{x}, —C(O)NR^{x}R^{y}, —NR^{x}C(O)R^{y}, —NR^{x}C(O)OR^{y}, —NR^{x}C(O)NR^{y}R^{z}, —NR^{x}SO₂R^{y}, —NR^{x}R^{y}, SR₁₁, SOR₁₁, SO₂, C₃-C₈ cycloalkyl, and 4-8 membered heterocycloalkyl; where each R^{x}, R^{y} and R^{z} is independently H or C₁-C₃ alkyl, or R^{x} and R^{y} may be taken together with the N to which they are attached form a 4-8 membered heterocycloalkyl, each optionally containing 1, 2 or 3 additional heteroatoms selected from O, N and S(O)_{q} where q is 0-2; wherein each said C₃-C₈ cycloalkyl, and 4-8 membered heterocycloalkyl is optionally substituted by 1 to 3 substituents independently selected from the group consisting of halo, —OH, oxo, C₁-C₃ alkyl, C₁-C₃ alkoxy, C₁-C₆ haloalkyl, C₁-C₃ hydroxyalkyl, —CN, —NH₂, —NH(C₁-C₃ alkyl), and —N(C₁-C₃ alkyl)₂.

In the case where optional substituents are selected independently from a list of alternatives, the selected groups may be the same or different. For example, in the case where a C₃-C₁₀ cycloalkyl or 4-8 membered heterocycloalkyl group is “optionally substituted from one or more independently selected R₉” (R₉ as defined herein); R₉ being the same or different may be substituted at any atom on the C₃-C₁₀ cycloalkyl or 4-8 membered heterocycloalkyl group, or two of the same R₉ may be substituted at two different atoms on the C₃-C₁₀ cycloalkyl or 4-8 membered heterocycloalkyl group, or two of the same R₉ may be substituted at the same atom on the C₃-C₁₀ cycloalkyl or 4-8 membered heterocycloalkyl group, or two of the same R₉ may be substituted at the same atom and a different R₉ may be substituted at a different atom on the C₃-C₁₀ cycloalkyl or 4-8 membered heterocycloalkyl group, or three different R₉ may be substituted at three different atoms on the C₃-C₁₀ cycloalkyl or 4-8 membered heterocycloalkyl group. For illustration purpose, a cyclopentyl group may be substituted by one F

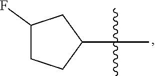 [SMILES: *C1CCC(F)C1]

may be substituted by two F's where each F being attached on a different carbon atom

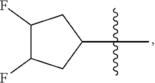 [SMILES: *C1CC(F)C(F)C1]

or may be substituted by two F's where both F's being attached on the same carbon atom and further substituted by a methyl being attached on a different carbon atom

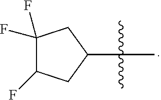 [SMILES: *C1CC(F)C(F)(F)C1]

Throughout the disclosure, it will be understood that the number and nature of optional substituent groups will be limited to the extent that such substitutions make chemical sense to one of ordinary skill in the art.

An optionally substituted group may be unsubstituted, partially substituted, or fully substituted, by one or more substituent(s) which can be the same or different. For example, an optionally substituted alkyl group may be unsubstituted (e.g., —CH₃, —CH₂CH₃), fully substituted (e.g., —CF₃, —CF₂CF₃), monosubstituted (e.g., —CH₂F, —CH₂CH₂F) or substituted at a level anywhere in between fully substituted and monosubstituted (e.g., —CHF₂, —CH₂CF₃). For example, an optionally substituted C₃-C₁₀ cycloalkyl may be unsubstituted (e.g., cyclopentolates), partially substituted

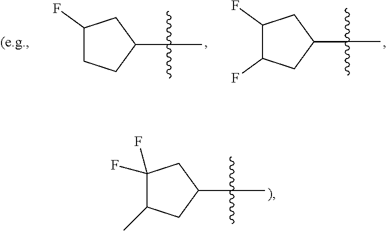

or fully substituted.

If substituents are described as being “independently selected” from a group, each substituent is selected independent of the other. Each substituent therefore may be identical to or different from the other substituent(s).

As used herein, the wavy line “ [symbol] ” that intersects a bond in a chemical structure refers to the point of attachment of the bond to which the wavy bond intersects in the chemical structure fragment to the remainder of a molecule or structural formula.

As used herein, the term “pharmaceutically acceptable” means the substance (e.g., the compounds described herein) and any salt thereof, or composition containing the substance or salt of the disclosure is suitable for administration to a subject or patient.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Salts

Salts encompassed within the term “pharmaceutically acceptable salts” refer to the compounds of this disclosure which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of the disclosure may also include other solvates of such compounds which are not necessarily pharmaceutically acceptable solvates, which may be useful as intermediates for one or more of the following: 1) preparing compounds of the disclosure; 2) purifying compounds of the disclosure; 3) separating enantiomers of compounds of the disclosure; or 4) separating diastereomers of compounds of the disclosure.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the disclosure may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two-dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers.

The compounds of the formulae provided herein may have asymmetric carbon atoms. The carbon-carbon bonds of the compounds of the disclosure may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the disclosure may contain more than one asymmetric carbon atom. In those compounds, the use of a solid line to depict bonds to asymmetric carbon atoms is meant to indicate that all possible stereoisomers are meant to be included and the attached stereocenter. For example, unless stated otherwise, it is intended that the compounds of the disclosure can exist as enantiomers and diastereomers or as racemates and mixtures thereof. The use of a solid line to depict bonds to one or more asymmetric carbon atoms in a compound of the disclosure and the use of a solid or dotted wedge to depict bonds to other asymmetric carbon atoms in the same compound is meant to indicate that a mixture of diastereomers is present.

The pharmaceutically acceptable salts of compounds of the disclosure may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

### Isotopes

The present disclosure includes all pharmaceutically acceptable isotopically-labeled compounds of the disclosure wherein one or more atoms are replaced by atoms having the same atomic number, but an atomic mass or mass number different from the atomic mass or mass number which predominates in nature.

Examples of isotopes suitable for inclusion in the compounds of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O phosphorus, such as ³²P, and sulfur, such as ³⁵S.

Certain isotopically-labelled compounds of the disclosure, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection.

Substitution with positron emitting isotopes, such as ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, the deuterium compound is selected from any one of the compounds set forth in Tables 11-13 shown in the Examples section.

In some embodiments, one or more hydrogen atoms on certain metabolic sites on the compounds of the invention are deuterated.

Isotopically-labeled compounds of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

### Prodrugs

A compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the disclosure may, for example, be produced by replacing appropriate functionalities present in compounds of the disclosure with certain moieties known to those skilled in the art as ‘pro-moieties’ as described, for example, in ‘Design of Prodrugs’ by H. Bundgaard (Elsevier, 1985).

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in a compound of the disclosure; (d) a thioester, thiocarbonate, thiocarbamate or sulfide derivatives of a thiol group when present in a compound of the disclosure; or (e) an oxime or imine derivative of a carbonyl group when present in a compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the disclosure contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the disclosure contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the disclosure contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the disclosure may themselves act as prodrugs of other compounds the disclosure It is also possible for two compounds of the disclosure to be joined together in the form of a prodrug. In certain circumstances, a prodrug of a compound of the disclosure may be created by internally linking two functional groups in a compound of the disclosure, for instance by forming a lactone.

### Metabolites

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (-Ph→-PhOH);
(vi) where the compound of the disclosure contains an amide group, a carboxylic acid derivative thereof (—CONH₂→COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the disclosure comprises pharmaceutical compositions. For pharmaceutical composition purposes, the compound per se or pharmaceutically acceptable salt thereof will simply be referred to as the compounds of the disclosure.

The compositions of this disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. In another embodiment, the oral administration may be in a powder or granule form. In another embodiment, the oral dosage form is sub-lingual, such as, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, ²0th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

### Administration and Dosing

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the disclosure may also be administered topically to the skin or mucosa, that is, dermally or transdermally. In another embodiment, the compounds of the disclosure may also be administered intranasally or by inhalation. In another embodiment, the compounds of the disclosure may be administered rectally or vaginally. In another embodiment, the compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. It is not uncommon that the administration of the compounds of the disclosure will be repeated a plurality of times in a day (typically no greater than 4 times). Multiple doses per day typically may be used to increase the total daily dose, if desired.

### Therapeutic Methods and Uses

The compounds of the disclosure may inhibit the activities of Cbl-B and may be useful in the treatment, prevention, suppression and amelioration of disease(s) such as cancers, disorders and conditions mediated by CBL-B. In particular, such compounds show an affinity for the CBL-B which is greater than their affinity for the C-CBL.

In some embodiments, the compounds of the disclosure are better inhibitors of CBL-B than for C-CBL.

The term “selective”, when used herein to describe a functionally-defined receptor ligand or enzyme inhibitor means selective for the defined receptor or enzyme subtype as compared with other receptor or enzyme subtypes in the same family. For instance, a selective CBL-B inhibitor is a compound which inhibits CBL-B more potently than C-CBL. In some embodiments, the binding affinity for CBL-B is at least 1-fold, for example, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 50-fold, 60-fold, 75-fold, 100-fold, or 100-fold larger than the binding affinity for C-CBL (as measured using conventional binding assays, for example, as any of those described herein).

In one aspect, the disclosure provides a method for the treatment of immunosuppression-associated disorders, such as chronic viral infections. Examples of such chronic viral infection include, but are not limited to, human immunodeficiency virus (HIV), hepatitis C virus (HCV), herpes virus infections, viral hepatitis, papillomas (warts), and papovavirus (including human papillomavirus (HPV)).

In one aspect, the disclosure provides a method for the treatment of abnormal cell growth in a subject comprising administering to the subject a therapeutically effective amount of a compound of the disclosure, or a pharmaceutically acceptable salt thereof. In frequent embodiments, the abnormal cell growth is cancer. In another aspect, the disclosure provides a method of inhibiting cancer cell proliferation in a subject, comprising administering to the subject a compound of the disclosure, or a pharmaceutically acceptable salt thereof, in an amount effective to inhibit cell proliferation.

In another aspect, the disclosure provides a method of inhibiting cancer cell invasiveness in a subject, comprising administering to the subject a compound of the disclosure, or a pharmaceutically acceptable salt thereof, in an amount effective to inhibit cell invasiveness.

In another aspect, the disclosure provides a method of inducing apoptosis in cancer cells in a subject, comprising administering to the subject a compound of the disclosure, or a pharmaceutically acceptable salt thereof, in an amount effective to induce apoptosis.

In some embodiments of the methods provided herein, the abnormal cell growth is cancer, wherein the cancer is selected from the group consisting of breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, endometrial cancer, vulval cancer, kidney cancer (including RCC), liver cancer (including HCC), pancreatic cancer, stomach (i.e., gastric) cancer, thyroid cancer, basal cell carcinomas, Hodgkin's lymphoma, non-Hodgkin's lymphoma, lymphoblastic leukemia, lymphocytic leukemia, acute myeloid leukemia (AML), multiple myeloma, melanoma, chondrosarcoma, neuroblastoma, glioblastoma multiforme, cervical cancer, and brain cancer.

### Co-Administration

The compounds of the disclosure may be used alone, or in combination with one or more other therapeutic agents. The disclosure provides any of the uses, methods or compositions as defined herein wherein the compound of the disclosure, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more other therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

Classes of additional chemotherapeutic agents, which can be administered in combination with a compound of this disclosure, include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists; IL-2 receptor agonist (recombinant cytokines or agonists for cytokine receptors); and anti-sense oligonucleotides or oligonucleotides derivatives that inhibit expression of genes implicated in abnormal cell proliferation or tumor growth.

Other additional chemotherapy agents include not only taxanes or platinum agents but also HER2 targeted agents, e.g., trastuzumab.

In another embodiment, such additional anti-cancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, spindle poison plant alkaloids, MCT4 inhibitors; MAT2a inhibitors; alk/c-Met/ROS inhibitors (including crizotinib or lorlatinib); mTOR inhibitors (including temsirolimus or gedatolisib); src/abl inhibitors (including bosutinib); cyclin-dependent kinase (CDK) inhibitors (including palbociclib); erb inhibitors (including dacomitinib); PARP inhibitors (including talazoparib); SMO inhibitors (including glasdegib); EGFR T790M inhibitors; PRMT5 inhibitors; TGFβR1 inhibitors; growth factor inhibitors; cell cycle inhibitors, biological response modifiers; enzyme inhibitors; and cytotoxics.

In another embodiment, such additional anti-cancer therapeutic agents include compounds derived from an anti-angiogenesis agent, including for example tyrosine kinase/vascular endothelial growth factor (VEGF) receptor (VEGFR) inhibitors (including sunitinib, axitinib, sorafenib, and tivozanib), TIE-2 inhibitors, PDGFR inhibitors, angiopoetin inhibitors, PKCs inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™), axitinib (Inlyta™), SU 14813 (Pfizer), and AG 13958 (Pfizer). Additional anti-angiogenesis agents include vatalanib (CGP 79787), pegaptanib octasodium (Macugen™), vandetanib (Zactima™), PF-0337210 (Pfizer), SU 14843 (Pfizer), AZD 2171 (AstraZeneca), ranibizumab (Lucentis™) Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™) valdecoxib (Bextra™), rofecoxib (Vioxx™), iguratimod (Careram™), IP 751 (Invedus), SC-58125 (Pharmacia) and etoricoxib (Arcoxia™). Yet further anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™) naproxen (Aleve™, Naprosyn™), diclofenac (Voltaren™), indomethacin (Indocin™) sulindac (Clinoril™), tolmetin (Tolectin™), etodolac (Lodine™), ketorolac (Toradol™) and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon). Yet further anti-angiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment, such additional anti-cancer therapeutic agents include compounds derived from hormonal agents and antagonists. Examples include where anti-hormonal agents act to regulate or inhibit hormone action on tumors such as anti-estrogens and selective estrogen receptor modulators (SERMs), and a selective estrogen receptor degrader (SERD) including tamoxifen, raloxifene, droloxifene, 4-hydroxytamoxifen, trioxifene, keoxifene, LY117018, onapristone, toremifene (Fareston), and fulvestrant. Examples also include aromatase inhibitors that inhibit the enzyme aromatase, which regulates estrogen production in the adrenal glands, and include compounds like 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin.

In another embodiment, such additional anti-cancer therapeutic agents include compounds derived from signal transduction inhibitors, such as inhibitors of protein tyrosine kinases and/or serine/threonine kinases: a signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK (including binimetinib (Mektovi™)), c-Kit inhibitors, FLT-3 inhibitors, K-Ras inhibitors, PI3 kinase inhibitors, JAK inhibitors, STAT inhibitors, Raf kinase inhibitors, BRAF (including encorafenib (Braftovi™)), Akt inhibitors, mTOR inhibitor, P70S6 kinase inhibitors, inhibitors of the WNT pathway and multi-targeted kinase inhibitors.

In another embodiment, such additional anti-cancer therapeutic agents include docetaxel, paclitaxel, paclitaxel protein-bound particles, cisplatin, carboplatin, oxaliplatin, capecitabine, gemcitabine or vinorelbine.

In another embodiment, such additional anti-cancer therapeutic agents include compounds derived from an epigenetic modulator, where examples include an inhibitor of EZH2 (including PF-06821497), SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In another embodiment, such additional anti-cancer therapeutic agents include compounds that are immuno-oncology agents, including immunomodulatory agents.

In another embodiment, combinations with pattern recognition receptors (PRRs) are contemplated. PRRs are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein.

The STING protein functions as both a cytosolic DNA sensor and an adaptor protein in Type 1 interferon signaling. The terms “STING” and “stimulator of interferon genes” refer to any form of the STING protein, as well as variants, isoforms, and species homologs that retain at least a part of the activity of STING. Unless indicated differently, such as by specific reference to human STING, STING includes all mammalian species of native sequence STING, e.g., human, monkey, and mouse STING is also known as—TMEM173.

“STING agonist” as used herein means, any molecule, which upon binding to STING, (1) stimulates or activates STING, (2) enhances, increases, promotes, induces, or prolongs an activity, function, or presence of STING, or (3) enhances, increases, promotes, or induces the expression of STING. STING agonists useful in the any of the treatment method, medicaments and uses of the present disclosure include, for example, nucleic acid ligands which bind STING.

Examples of STING agonists that are useful in the treatment methods, medicaments, and uses of the present disclosure include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as WO2019027858, WO20180093964, WO2017175156, WO2017175147.

Therapeutic antibodies may have specificity against a variety of different antigens. For example, therapeutic antibodies may be directed to a tumor associated-antigen, such that binding of the antibody to the antigen promotes death of the cell expressing the antigen. In other example, therapeutic antibodies may be directed to an antigen on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

In another embodiment, such additional anti-cancer therapeutic agents include antibodies that would be blocking or inhibitory at the target: CTLA-4 (including ipilimumab or tremelimumab), PD-1 or PD-L₁ (including atezolizumab, avelumab, cemiplimab, durvalumab, nivolumab, sasanlimab, or pembrolizumab), LAG-3, TIM-3, or TIGIT.

In another embodiment, such additional anti-cancer therapeutic agents include antibodies that are agonists of 4-1BB, OX40, GITR, ICOS, or CD40.

In another embodiment the anti-cancer therapy may be a CAR-T-cell therapy.

Examples of a therapeutic antibody include: an anti-OX40 antibody, an anti-4-1BB antibody, an anti-HER2 antibody (including an anti-HER2 antibody-drug conjugate (ADC)), a bispecific anti-CD47/anti-PD-L₁ antibody, and a bispecific anti-P-cadherin/anti-CD3 antibody. Examples of cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof. Exemplary immunomodulating agents that may be incorporated in an ADC include gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-15, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta. and -.gamma), the stem cell growth factor designated “S 1 factor,” erythropoietin and thrombopoietin, or a combination thereof.

Additional examples of therapeutic antibodies may include the following antigens where exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. see U.S. Pat. No. 9,969,809]; BTN1A1 (e.g. see WO2018222689); CA-125 (e.g. abagovomab); Carboanhydrase IX; CCR2; CCR4 (e.g. mogamulizumab); CCR5 (e.g. Ieronlimab); CCR8; CD3 [e.g. blinatumomab (CD3/CD19 bispecific), CD3/P-cadherin bispecific, CD3/BCMA bispecific] CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD123; CD276/B7-H3 (e.g. omburtamab); CDH17; CEA; ClhCG; CTLA-4 (e.g. ipilimumab, tremelimumab), CXCR₄; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); GD2 (e.g. dinutuximab, 3F8); GD3; GITR; GloboH; GM1; GM2; HER2/neu [e.g. margetuximab, pertuzumab, trastuzumab; ado-trastuzumab emtansine, trastuzumab duocarmazine, [see U.S. Pat. No. 8,828,401]; HER3; HER4; ICOS; IL-10; ITG-AvB6; LAG-3 (e.g. relatlimab); Lewis-Y; LG; Ly-6; M-CSF [see U.S. Pat. No. 7,326,414]; MCSP; mesothelin; MUC1; MUC2; MUC3; MUC4; MUC5AC; MUC5B; MUC7; MUC16; Notch1; Notch3; Nectin-4 (e.g. enfortumab vedotin); OX40 [see U.S. Pat. No. 7,960,515]; P-Cadherein [see WO2016/001810]; PCDHB2; PDGFRA (e.g. olaratumab); Plasma Cell Antigen; PolySA; PSCA; PSMA; PTK7 [see U.S. Pat. No. 9,409,995]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7,10-tetraazacycIododecane-N,N′,N″,N′″-tetraacetic acid (DOTA), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

The disclosure also concerns the use of radiation in combination with any anti-cancer therapeutic agent administered herein. More specifically, compounds of the disclosure can be administered in combination with additional therapies, such as radiation therapy and/or chemotherapy.

These agents and compounds of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the disclosure provides kits comprising the compound of the disclosure or pharmaceutical compositions comprising the compound of the disclosure. A kit may include, in addition to the compound of the disclosure or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents.

In yet another embodiment, the disclosure comprises kits that are suitable for use in performing the methods of treatment described herein. In one embodiment, the kit contains a first dosage form comprising one or more of the compounds of the disclosure in quantities sufficient to carry out the methods of the disclosure. In another embodiment, the kit comprises one or more compounds of the disclosure in quantities sufficient to carry out the methods of the disclosure and a container for the dosage and a container for the dosage.

### Synthetic Methods

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the disclosure. It will be further appreciated that it may be necessary or desirable to carry out the transformations in a different order from that described in the schemes, or to modify one or more of the transformations, to provide the desired compound of the disclosure.

In the preparation of compounds of the disclosure it is noted that some of the preparation methods useful for the preparation of the compounds described herein may require protection of remote functionality (e.g., a primary amine, secondary amine, carboxyl, etc. in a precursor of a compound of the disclosure). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the disclosure.

### General Experimental Details

¹H and ¹⁹F Nuclear Magnetic Resonance (NMR) spectra were recorded on Bruker XWIN-NMR (400 or 700 MHz) spectrometer. ¹H and ¹⁹F resonances are reported in parts per million (ppm) downfield from tetramethylsilane. ¹H NMR data are reported as multiplicity (e.g., s, singlet; d, doublet; t, triplet; q, quartet; quint, quintuplet; dd, doublet of doublets; dt, doublet of triplets; br s, broad singlet; m, multiplet). For spectra obtained in CDCl₃, DMSO-d₆, and CD₃OD, the residual protons (7.27, 2.50, and 3.31 ppm, respectively) were used as the internal reference. All observed coupling constants, J, are reported in Hertz (Hz). “δ” means chemical shift. Exchangeable protons are not always observed.

Optical rotations were determined on a Jasco P-2000 or a Rudolph Autopol IV polarimeter. All final compounds were purified to 95% purity, unless otherwise specified. When absolute stereochemistry is known, (R,S) labels are used. When absolute stereochemistry is not known, the software-generated names are modified to include the symbol (ξ) indicating one single isomer with unknown stereochemistry, and the chemical structures are modified to include “or 1” at the chiral center where the stereochemistry is not known.

Mass spectra, MS (m/z), were recorded using either electrospray ionization (ESI) or atmospheric pressure chemical ionization (APCI). Where relevant and unless otherwise stated, the m/z data provided are for isotopes ¹⁹F, ³⁵Cl, ⁷⁹Br and ¹²⁷I.

The nomenclature is written as described by IUPAC (International Union of Pure and Applied Chemistry generated within Perkin Elmers Chemdraw 18.0.0.231. The naming convention provided with Perkin Elmers Chemdraw 18.0.0.231 is well known by those skilled in the art and it is believed that the naming convention provided with Perkin Elmers Chemdraw 18.0.0.231 generally comports with the IUPAC (International Union for Pure and Applied Chemistry) recommendations on Nomenclature of Organic Chemistry and the CAS Index rules.

### Abbreviations

The following abbreviations are used throughout the Examples: “ACN” means acetonitrile, “aq” means aqueous, “atm” means atmosphere(s), “BOC”, “Boc” or “boc” means N-tert-butoxycarbonyl, “Boc₂O” means di-tert-butyl dicarbonate, “Bn” means benzyl, “Bu” means butyl, “nBu” means normal-butyl, “tBu” means tert-butyl, “t-BuOK” means potassium tert-butoxide, “c” means concentration, “CDCl₃” means deuterated chloroform, “CO₂” means carbon dioxide, “DCM” (CH₂Cl₂) means methylene chloride, “de” means diastereomeric excess, “DEA” means diethylamine, “DIPEA” means diisopropyl ethyl amine, “DME” means 1,2-dimethoxyethane, “DMF” means N,N-dimethyl formamide, “DTT” means dithiothreitol, “DMSO” means dimethylsulfoxide, “EA” means ethyl acetate, “ESI” means electrospray ionization, “ee” means enantiomeric excess, “equiv” means equivalent, “Et” means ethyl, “EtOAc” means ethyl acetate, “EtOH” means ethanol, “HOAc” or “AcOH” means acetic acid, “HCl” means hydrochloric acid, “H₂O” means water, “HPLC” means high pressure liquid chromatography, “i-Pr” or “iPr” means isopropyl, “IPA” means isopropyl alcohol, “KOH” means potassium hydroxide, “LAH” means lithium aluminum hydride, “LCMS” means liquid chromatography mass spectrometry, “LHMDS” means lithium hexamethyldisilazide (lithium bis(trimethylsilyl)amide), “Me” means methyl, “MeCN” means acetonitrile, “MeOH” means methanol, “mg” means milligram, “MHz” means mega Hertz, “mL” means milliliter, “mmol” means millimole, “MS” means mass spectrometry, “MTBE” means methyl tert-butyl ether, “PE” means petroleum ether, “NaHCO₃” means sodium bicarbonate, “Na₂SO₄” means sodium sulfate, “NCS” means N-chlorosuccinimide, “Ph” means phenyl, “SiO₂” means silica, “RT” means room temperature, “TBME” means tert-Butyl methyl ether, “TFA” means trifluoroacetic acid, “THF” means tetrahydrofuran, “SFC” means supercritical fluid chromatography, “TLC” means thin layer chromatography, “Rf” means retention factor, “˜” means approximately, “rt” means retention time, “h” means hour, “min” means minute, “equiv” means equivalents, “sat.” means saturated.

The schemes described below are intended to provide a general description of the methodology employed in the preparation of the compounds of the present disclosure. Some of the compounds of the present disclosure contain a single chiral center. In the following schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### General Methods

Unless stated otherwise, the variables in Schemes I-XIV have the same meanings as defined herein.

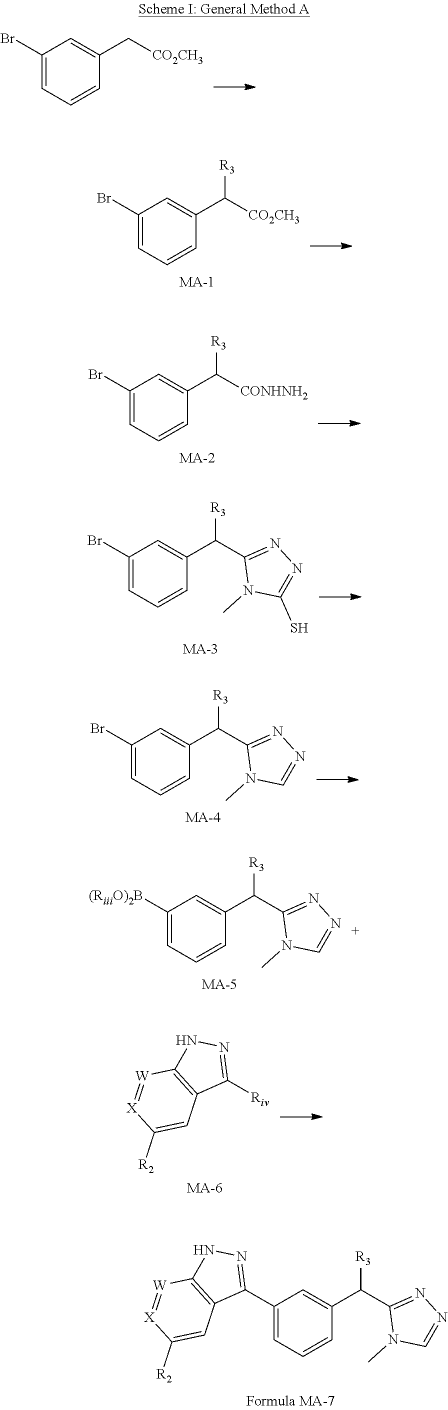

Method A refers to a synthetic sequence for the preparation of compounds of Formula MA-7, as depicted above. Alkylation of methyl (3-bromophenyl)acetate with alkyl or cycloalkyl halides under basic conditions (t-BuOK) provides the ester MA-1. Hydrazinolysis of MA-1 with hydrazine to the acetohydrazide MA-2, followed by addition to isothiocyanatomethane and cyclization gives triazole-3-thiol MA-3. Desulfurization of MA-3 either under oxidative or diazotative conditions yields the bromo triazole MA-4. Borylation of MA-4 gives the boronate ester MA-5 (Rᵢᵢᵢ can be H, or RᵢᵢᵢO)₂B=pinacolato boron “B(pin)”) which upon coupling with a 3-halo pyrazolopyridine, MA-6 (Rᵢᵥ=Br, I; W=N, CH or C-Me; X=N or CH; R₂=H, halogen, alkyl or CF₃) under standard cross-coupling conditions provides pyrazolo[3,4-c]pyridine Formula MA-7.

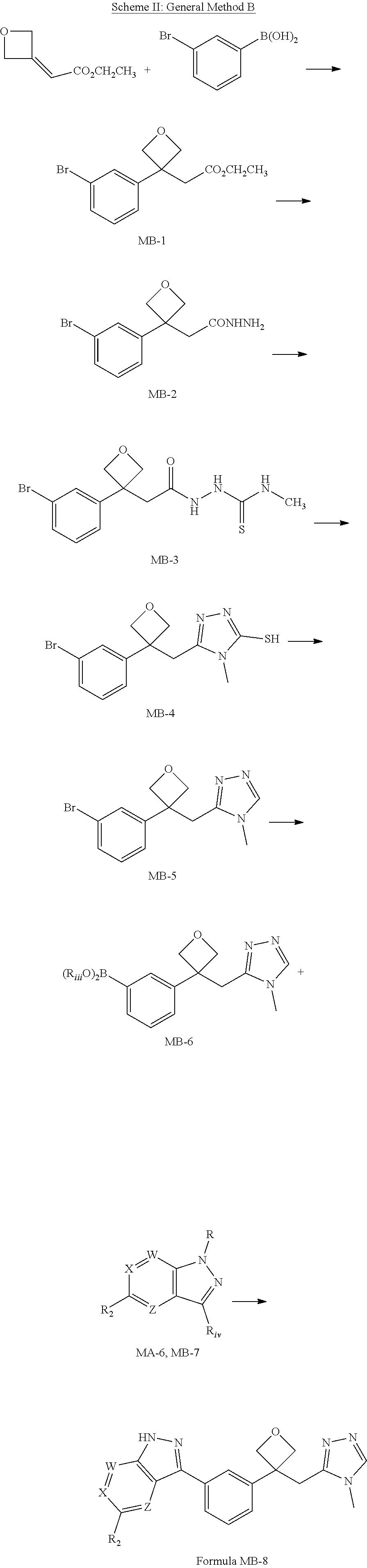

Method B refers to a synthetic sequence for the preparation of compounds of Formulas MB-9, MB-10, and MB-11, as depicted above. A rhodium (1) catalyzed Hayashi-Miyaura reaction of ethyl 2-(oxetan-3-ylidene)acetate with (3-bromophenyl)boronic acid yields the bromide MB-1. Hydrazinolysis of MB-1 with hydrazine to the acetohydrazide MB-2, addition to isothiocyanatomethane to yield the N-methylhydrazine-1-carbothioamide, MB-3, followed by cyclization gives the triazole-3-thiol, MB-4. Desulfurization of MB-4 under diazotative conditions yields the bromo triazole MB-5. Borylation of MB-5 to the boronate ester MB-6 followed by coupling under standard cross-coupling conditions with either 3-halo pyrazolopyridine MA-6, or 3-halo-imidazole or 3-halo-pyrazolo[3,4-b]pyridine MB-7 (Rᵢᵥ=Br, I; W, X, and Z can be independently C—Rᵢᵢᵢ or N, where Rᵢᵢᵢ is H, halogen, alkyl, cycloalkyl, alkoxy, or CF₃) to provide Formula MB-8. In some cases, a compound such as MB-7 may contain protecting groups (Boc) which are added and removed by additional steps, using conditions known in the art.

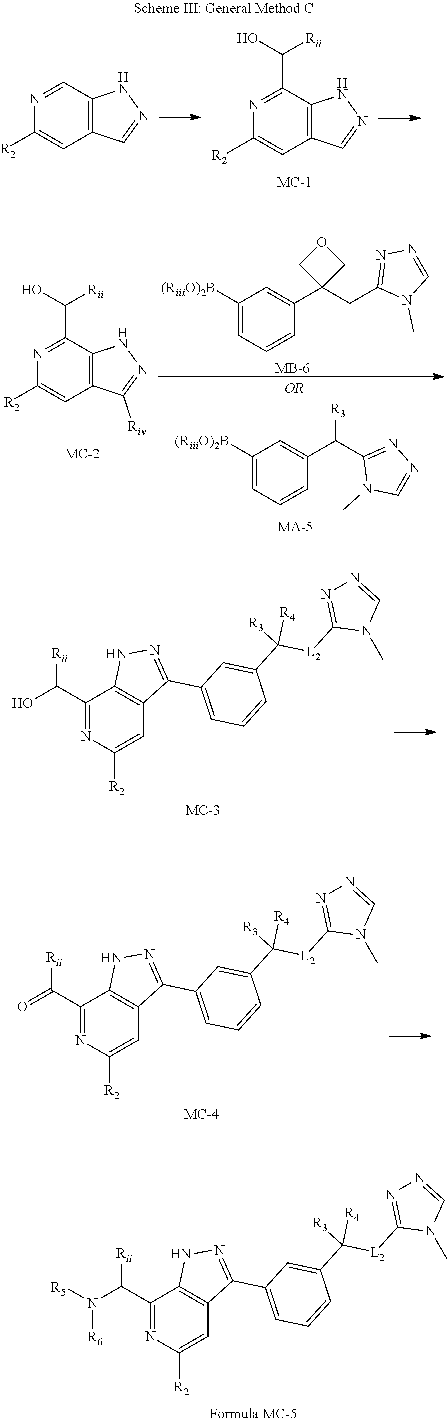

Method C refers to a synthetic sequence for the preparation of compounds of Formula MC-5, as depicted above. A Minisci-type hydroxymethylation of 5-(trifluoromethyl)-1H-pyrazolo[3,4-c]pyridine or 1H-pyrazolo[3,4-c]pyridine using either methanol (Rᵢᵢ=H) or ethanol (Rᵢᵢ=Me) and a suitable oxidant (ammonium persulfate) provides the 7-hydroxymethyl pyrazolo[3,4-c]pyridine MC-1 (Rᵢᵢ=H, Me; R₂=H, CF₃). Halogenation using an N-halosuccinimide yields the corresponding 3-halo-pyrazolo[3,4-c]pyridine MC-2 (Rᵢᵥ=Br, I). Coupling of MC-2 with MB-6 or MA-5 under standard cross-coupling conditions gives the alcohol MC-3 (R₃ and R₄ together with the carbon atom to which they are attached to form an oxetane, or R₃=C₁-C₆ cycloalkyl and R₄=H; L₂=CH₂ or a bond). Oxidation of MC-3 provides the ketone MC-4 correspondingly. Reductive amination of ketone or aldehyde MC-4 yields the amine Formula MC-5.

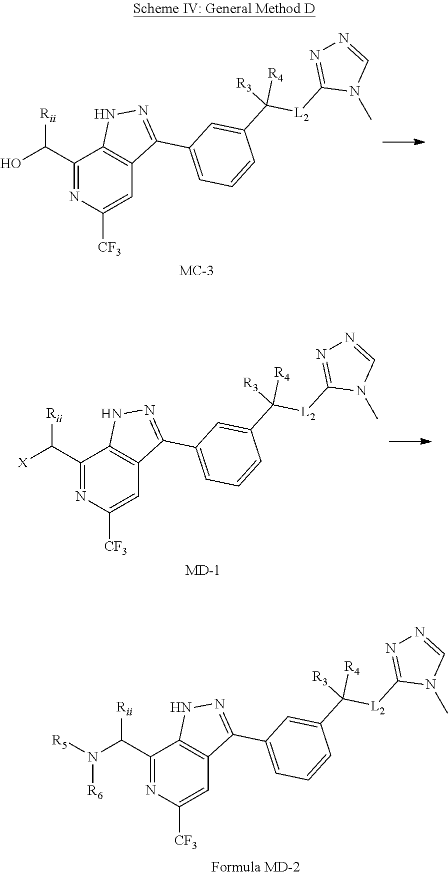

Method D refers to a synthetic sequence for the preparation of compounds of Formula MD-2, as depicted above. Halogenation of alcohol MC-3 under standard conditions gives the halide MD-1 (Rᵢᵥ=Br, I; R₃ and R₄ together with the carbon atom to which they are attached to form an oxetane, or R₃=C₁-C₆ cycloalkyl and R₄=H; L₂=CH₂ or a bond) followed by displacement with a suitable amine under basic conditions provides the pyrazolo[3,4-c]pyridine Formula MD-2. In cases where amines were protected at a distal position, the protecting groups were removed and the amines could be N-alkylated under standard conditions known in the art.

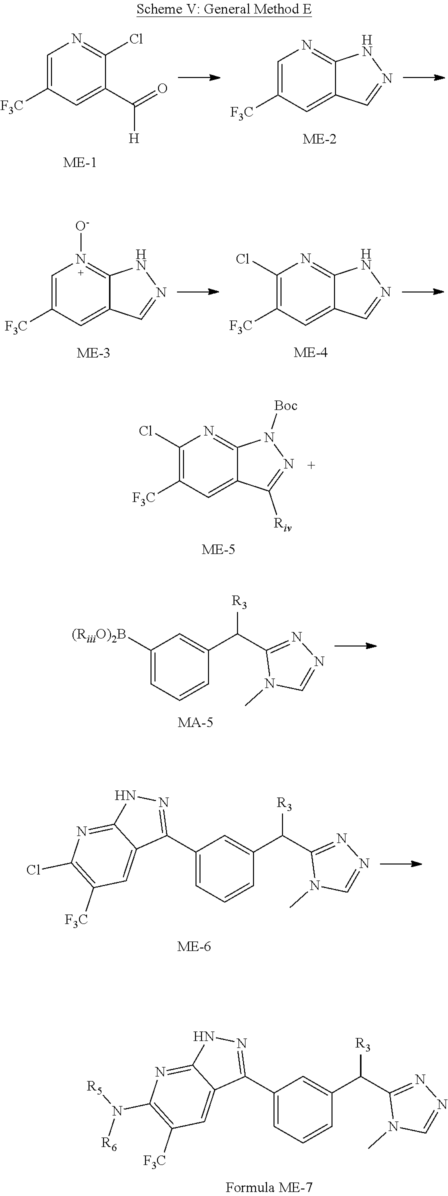

Method E refers to a synthetic sequence for the preparation of compounds of Formula ME-7, as depicted above. Condensation of 2-chloro-5-(trifluoromethyl)nicotinaldehyde (ME-1) with hydrazine yields the pyrazolo[3,4-b]pyridine ME-2. Conversion to the N-oxide ME-3 followed by chlorination under standard conditions (such as tosic anhydride, tetrabutylammonium chloride) and protection (such as Boc₂O) provides the chloride ME-5 (Rᵢᵥ=Br, I). Coupling with MA-5 under Suzuki cross-coupling conditions and concomitant deprotection of the Boc group under those conditions gives the 6-chloro-pyrazolo[3,4-b]pyridine ME-6. A nucleophilic aromatic substitution reaction of ME-6 with a suitable amine under basic conditions (such as triethylamine) yields the 6-amino-pyrazolo[3,4-b]pyridine Formula ME-7.

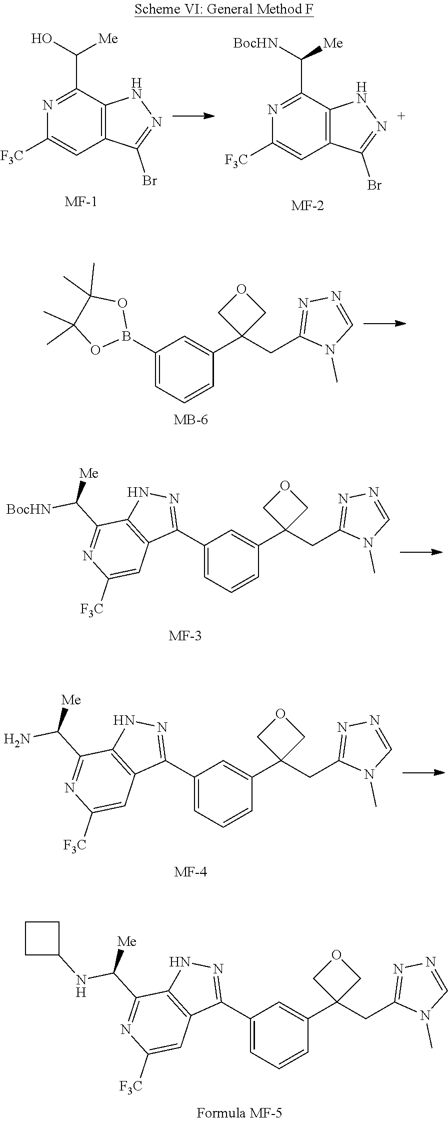

Method F refers to a synthetic sequence for the preparation of compounds of Formula MF-5, as depicted above. A ruthenium-catalyzed hydrogen borrowing strategy starting from the racemic alcohol MF-1 using a chiral sulfinamide as an auxiliary gives access to the α-chiral amine MF-2 (J. Am. Chem. Soc. 2014, 136, 12548) after deprotection of the intermediate sulfinamide and subsequent Boc-protection. Coupling of MF-2 with boronate MB-6 under Suzuki cross-coupling conditions provides the Boc-protected amine MF-3. Deprotection under standard conditions gives the amine MG-4 which underwent a reductive amination with a ketone to yield the amine Formula MF-5.

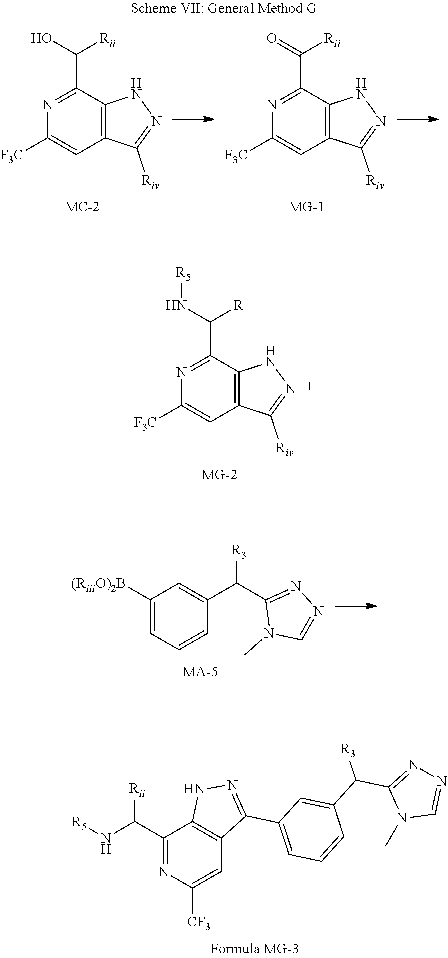

Method G refers to a synthetic sequence for the preparation of compounds of Formula MG-3, as depicted above. Oxidation of the alcohol MC-2 (Rᵢᵢ=H or Me, Rᵢᵥ=Br or 1) to the aldehyde MG-1 under standard conditions followed by reductive amination with suitable amines provides the 7-aminoalkyl-pyrazolo[3,4-c]pyridine MG-2. Protection under standard conditions (such as Boc₂O), followed by coupling with the boronate MA-5 and a subsequent deprotection under standard conditions yield the pyrazolo[3,4-c]pyridine Formula MG-3.

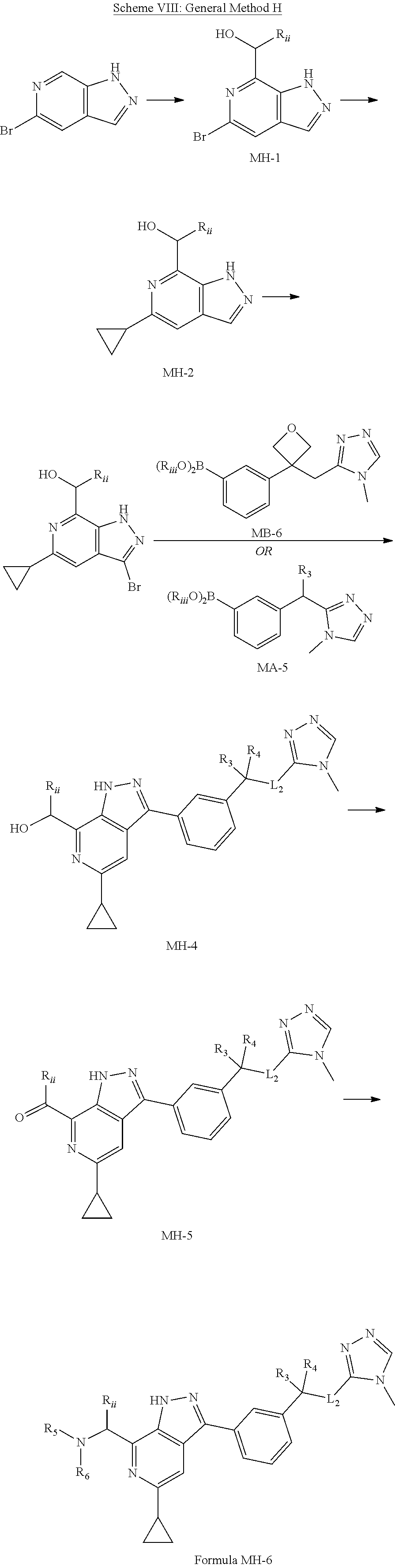

Method H refers to a synthetic sequence for the preparation of compounds of Formula MH-6, as depicted above. A Minisci-type hydroxymethylation of 5-bromo-1H-pyrazolo[3,4-c]pyridine using either methanol (Rᵢᵢ=H) or ethanol (Rᵢᵢ=Me) and a suitable oxidant (ammonium persulfate) provides the 7-hydroxymethyl pyrazolo[3,4-c]pyridine MH-1 (Rᵢᵢ=H, Me). N-Protection followed by Suzuki coupling to install the cyclopropyl group and N-deprotection gives MH-2. Halogenation using a N-halosuccinimide yields the corresponding 3-halo-pyrazolo[3,4-c]pyridine MH-3. Coupling of MH-3 (Rᵢᵢ=H, Me) with MB-6 or MA-5 under standard cross-coupling conditions gives the alcohol MH-4 (R₃ and R₄ together with the carbon atom to which they are attached to form an oxetane, or R₃=C₁-C₆ cycloalkyl and R₄=H; L₂=CH₂ or a bond) followed by oxidation provides the ketones MH-5 correspondingly. Reductive amination of ketone MH-5 yields the amine Formula MH-6.

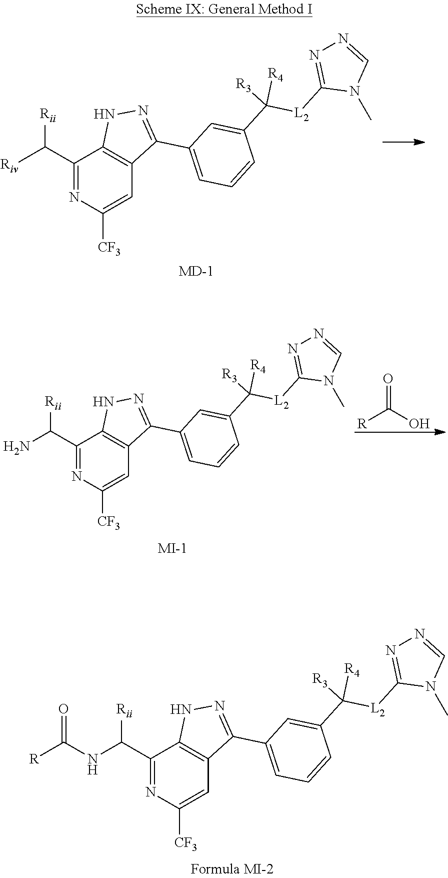

Method I refers to a synthetic sequence for the preparation of compounds of Formula MI-2, as depicted above. Amination of halogen MD-1 using ammonium hydroxide solution gives amine MI-1. Standard amide coupling conditions can be used to join a carboxylic acid and MI-1 to give amide Formula MI-2.

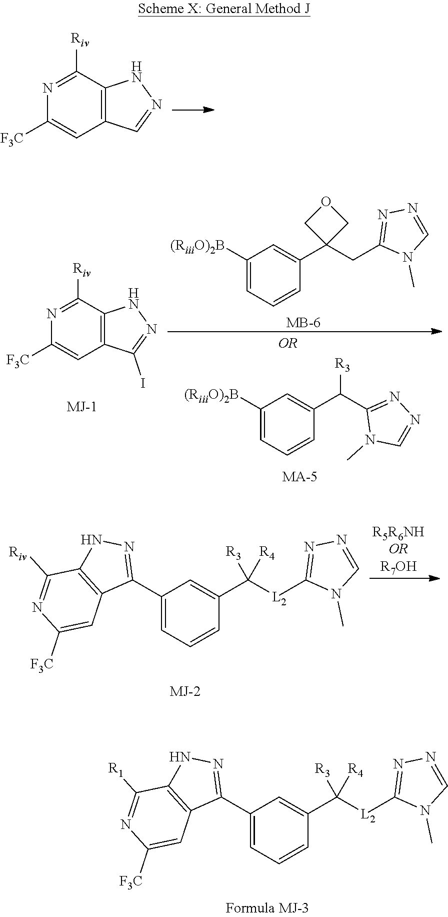

Method J refers to a synthetic sequence for the preparation of compounds of Formula MJ-3, as depicted above. Iodination of 7-halo-5-(trifluoromethyl)-1H-pyrazolo[3,4-c]pyridine using an N-iodosuccinimide yields the corresponding 3-halo-pyrazolo[3,4-c]pyridine MJ-1 (Rᵢᵥ=Br, Cl). Coupling of MJ-1 with MB-6 or MA-5 under standard cross-coupling conditions gives the halide MJ-2 (R₃ and R₄ together with the carbon atom to which they are attached to form an oxetane, or R₃=C₁-C₆ cycloalkyl and R₄=H; L₂=CH₂ or a bond). S_{N}Ar displacement of the halide using an alcohol or amine yields Formula MJ-3 (R₁=NR₅R₆ or OR₇). In some cases, a compound such as MJ-1 may contain protecting groups (SEM) which are added and removed by additional steps, using conditions known in the art.

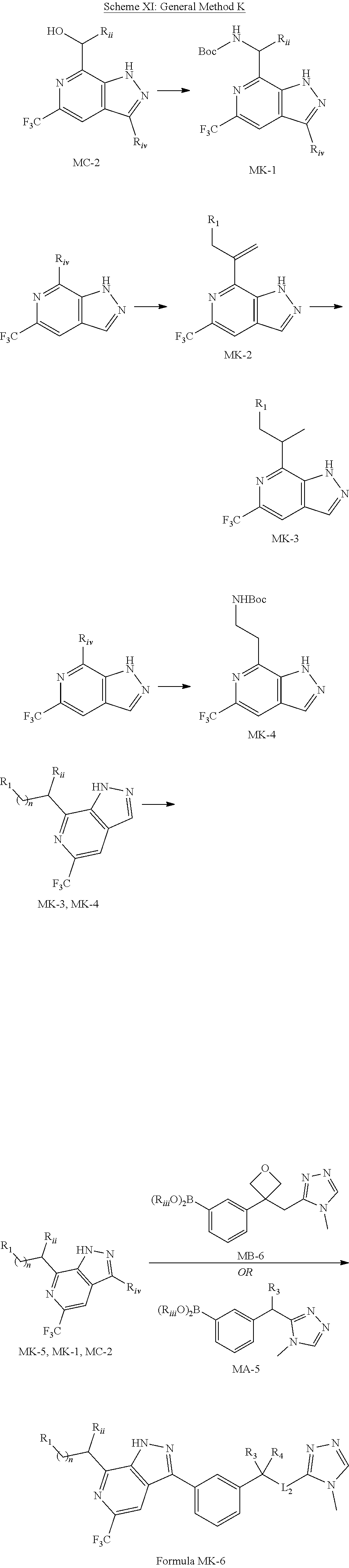

Method K refers to a synthetic sequence for the preparation of compounds of Formula MK-6, as depicted above. Alcohol MC-2 (Rᵢᵢ=H or Me, Rᵢᵥ=Br or 1) can be converted to amine MK-1 via a rhodium catalyzed hydrogen-borrowing amination. Additionally, 7-halo-pyrazolo[3,4-c]pyridines can undergo cross-coupling reactions via standard Suzuki conditions to give alkene MK-2 or alkane MK-4. Hydrogenation of the alkene MK-2 (R₁=NHBoc or OH) gives MK-3 (R₁=NHBoc or OH). Halogenation of pyrazolo[3,4-c]pyridines MK-3 or MK-4 (Rᵢᵢ=H or Me, R₁=NHBoc or OH, n=0 or 1) using an N-halosuccinimide yields the corresponding 3-halo-pyrazolo[3,4-c]pyridine MK-5 (Rᵢᵥ=Br, I, Rᵢᵢ=H or Me, R₁=NHBoc or OH, n=0 or 1). Coupling of pyrazolo[3,4-c]pyridines MK-5 (Rᵢᵢ=H or Me; Rᵢᵥ=Br, I; R₁=NHBoc; n=1), or MK-1 (Rᵢᵢ=H or Me; Rᵢᵥ=Br, I; R₁=NHBoc, n=0), or MC-2 (Rᵢᵢ=H or Me; Rᵢᵥ=Br, I; R₁═OH, n=0) with MB-6 or MA-5 under standard cross-coupling conditions followed by deprotection under standard conditions where applicable gives Formula MK-6 (R₃ and R₄ together with the carbon atom to which they are attached to form an oxetane, or R₃=C₁-C₆ cycloalkyl and R₄=H; L₂=CH₂ or a bond, Rᵢᵢ=H or Me, R₁=NHBoc, NH₂, or OH, n=0 or 1).

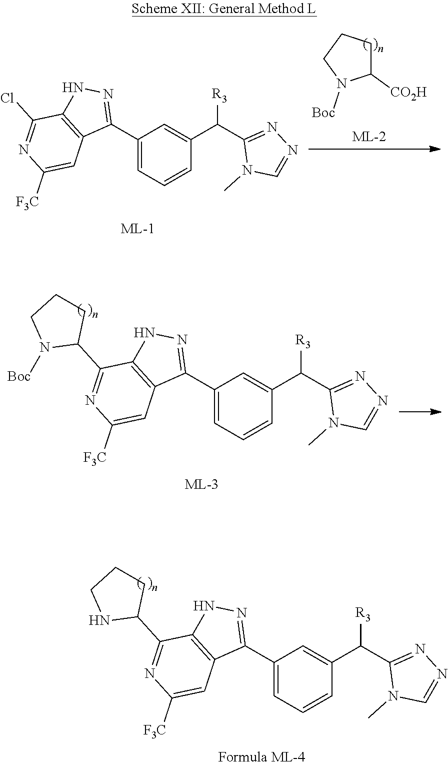

Method L refers to a synthetic sequence for the preparation of compounds of Formula ML-4, as depicted above. Beginning with chloride ML-1 (R₃=C₁-C₆ cycloalkyl), decarboxylative cross-coupling with carboxylic acids ML-2 (n=1 or 2) under photoredox catalysis conditions affords amines ML-3. Straightforward deprotection gives Formula ML-4.

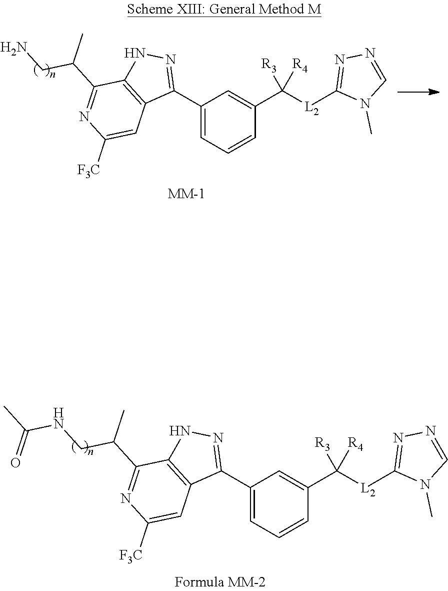

Method M refers to a synthetic sequence for the preparation of compounds of Formula MM-2, as depicted above. Amine MM-1 (n=0 or 1) is acetylated using acetic acid or acetyl chloride to afford amides Formula MM-2.

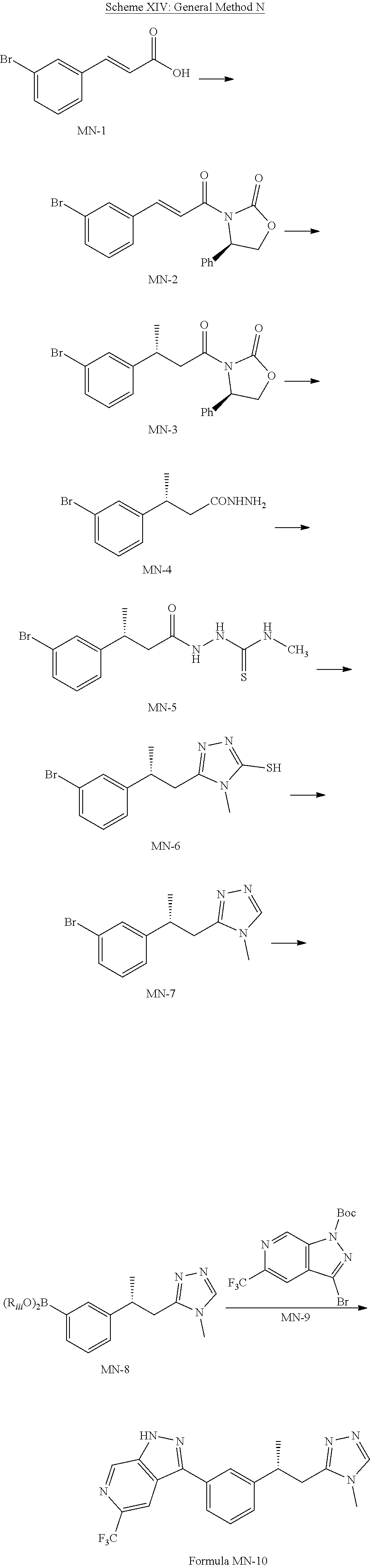

Method N refers to a synthetic sequence for the preparation of compounds of Formula MN-10, as depicted above. Activation of carboxylic acid MN-1 as the acyl chloride followed by amidation with Evans' oxazolidinone affords MN-2. Diastereoselective Michael addition of methylcuprate gives MN-3. Hydrazinolysis of MN-3 with hydrazine to the acetohydrazide MN-4, addition to isothiocyanatomethane to yield the N-methylhydrazine-1-carbothioamide, MN-5, followed by cyclization gives the triazole-3-thiol, MN-6. Desulfurization of MN-6 under oxidative conditions yields the bromo triazole MN-7. Borylation of MN-7 to the boronate ester MN-8 followed by coupling under standard cross-coupling conditions with 3-halo-pyrazolo[3,4-c]pyridine MN-9 provides pyrazolo[3,4-c]pyridine Formula MN-10. In some cases, a compound such as MN-9 may contain protecting groups (Boc) which are added and removed by additional steps, using conditions known in the art.

### Preparation of Intermediates

### Preparation of Intermediate 1: 3-{cyclobutyl[3-(4,4,5,5-tetramethyl-1,3,2-dioxaborolan-2-yl)phenyl]methyl}-4-methyl-4H-1,2,4-triazole

### Step 1: Methyl (3-bromophenyl)(cyclobutyl)acetate (1a)

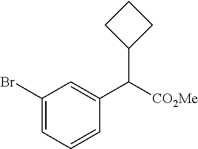 [SMILES: COC(=O)C(c1cccc(Br)c1)C1CCC1]

To a solution of methyl (3-bromophenyl)acetate (50.0 g, 218 mmol, 1.0 equiv) in DMF (450 mL) was added potassium tert-butoxide (31.8 g, 284 mmol, 1.3 equiv) portionwise at 0° C. The reaction was stirred at 0° C. for 30 min, then bromocyclobutane (35.4 g, 262 mmol, 1.2 equiv) in DMF (50 mL) was added dropwise at 0° C. The reaction was stirred at 25° C. for 18 h. The reaction was diluted with H₂O (500 mL) and EtOAc (400 mL) and the layers were separated. The aqueous layer was extracted with EtOAc (2×400 mL). The combined organic layers were washed with brine (4×200 mL), dried over Na₂SO₄, filtered, and the filtrate was concentrated under reduced pressure to give methyl (3-bromophenyl)(cyclobutyl)acetate (1a) (59 g, 96%) as a yellow oil. ¹H NMR (400 MHz, CDCl₃) δ 7.46 (d, 1H), 7.40 (dt, 1H), 7.26-7.16 (m, 2H), 3.70-3.65 (m, 3H), 3.52 (d, 1H), 3.02-2.93 (m, 1H), 2.30-2.13 (m, 1H), 1.94-1.76 (m, 4H), 1.65-1.55 (m, 1H).

### Step 2: 2-(3-bromophenyl)-2-cyclobutylacetohydrazide (1b)

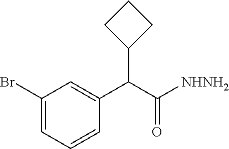 [SMILES: NNC(=O)C(c1cccc(Br)c1)C1CCC1]

To a solution of methyl (3-bromophenyl)(cyclobutyl)acetate (1a) (13.5 g, 47.7 mmol, 1.0 equiv) in EtOH (160 mL) was added hydrazine hydrate (42.1 g, 715 mmol, 15 equiv). The reaction was stirred at 80° C. for 18 h, then cooled and concentrated under reduced pressure. The crude residue was purified by flash column chromatography (ISCO, 80 g SiO₂, pet ether/EtOAc 0-100%) to give 2-(3-bromophenyl)-2-cyclobutylacetohydrazide (1b) (11 g, 81%) as a yellow gum. ¹H NMR (400 MHz, DMSO-d₆) δ 9.21 (br s, 1H), 7.49 (s, 1H), 7.40 (td, 1H), 7.31-7.20 (m, 2H), 4.21 (d, 2H), 3.36 (s, 1H), 2.98-2.84 (m, 1H), 2.05-1.96 (m, 1H), 1.84-1.61 (m, 4H), 1.53-1.43 (m, 1H).

### Step 3: 5-[(3-bromophenyl)(cyclobutyl)methyl]-4-methyl-4H-1,2,4-triazole-3-thiol (1c)

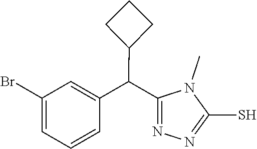 [SMILES: Cn1c(S)nnc1C(c1cccc(Br)c1)C1CCC1]

To a solution of 2-(3-bromophenyl)-2-cyclobutylacetohydrazide (1b) (11 g, 38.8 mmol, 1.0 equiv) in THF (150 mL) was added isothiocyanatomethane (3.98 g, 54.4 mmol, 1.4 equiv) and the reaction was stirred at 20° C. for 2 days. Then, sodium hydroxide (4.66 g, 117 mmol, 3.0 equiv) in H₂O (30 mL) was added and the reaction mixture was stirred at 60° C. for 3 h. The reaction was cooled and concentrated under reduced pressure to remove THF. H₂O (50 mL) was added, and the reaction was acidified to pH ˜1. The mixture was filtered, then the wet solid was dissolved in MeCN (2×50 mL) and concentrated under reduced pressure to give 5-[(3-bromophenyl)(cyclobutyl)methyl]-4-methyl-4H-1,2,4-triazole-3-thiol (1c) (13 g, 99%) as an off-white solid. ¹H NMR (400 MHz, DMSO-d₆) δ 7.52-7.40 (m, 2H), 7.34-7.27 (m, 1H), 7.26-7.19 (m, 1H), 4.22 (d, 1H), 3.19 (s, 3H), 2.99-2.88 (m, 1H), 1.88-1.59 (m, 6H).

### Step 4: 3-[(3-bromophenyl)(cyclobutyl)methyl]-4-methyl-4H-1,2,4-triazole (1d)

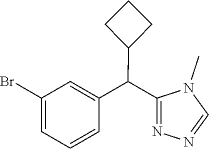 [SMILES: Cn1cnnc1C(c1cccc(Br)c1)C1CCC1]

To a solution of acetic acid (25 mL) in DCM (70 mL) was added hydrogen peroxide (30%, 21.8 g, 192 mmol, 5.0 equiv) slowly at 0-5° C. Then 5-[(3-bromophenyl)(cyclobutyl)methyl]-4-methyl-4H-1,2,4-triazole-3-thiol (1c) (13 g, 38.4 mmol, 1.0 equiv) in DCM (70 mL) was added slowly at 0-5° C. and then warmed to 15° C. After 2 h, the reaction was quenched with saturated sodium sulfite. The layers were separated, and the organic layer was concentrated to remove most of the solvent. H₂O (50 mL) was added, and the reaction was basified to pH ˜8 using saturated aqueous sodium carbonate. The mixture was extracted with EtOAc (2×80 mL). The combined organic layers were washed with brine (150 mL), dried over Na₂SO₄, filtered, and concentrated under reduced pressure. The crude residue was purified by flash column chromatography (ISCO, 120 g SiO₂, DCM/MeOH 0-10%) to give 3-[(3-bromophenyl)(cyclobutyl)methyl]-4-methyl-4H-1,2,4-triazole (1d) (6.76 g, 57%) as a yellow solid. ¹H NMR (400 MHz, DMSO-d₆) δ 8.33 (s, 1H), 7.46-7.39 (m, 2H), 7.31-7.24 (m, 2H), 4.22 (d, 1H), 3.41 (s, 3H), 3.14-3.04 (m, 1H), 2.09-1.99 (m, 1H), 1.83-1.64 (m, 5H).

The racemic compound could be purified into its enantiomers via preparative SFC (Column: Phenomenex Lux Cellulose-2 AXIA Pack, 250×21.2 mm, 5 um; Temperature: 35° C.; Pressure: 120 bar; Flow rate: 100 mL/min; 17% MeOH in CO₂). Peak 1: 3-[(S)-(3-bromophenyl)(cyclobutyl)methyl]-4-methyl-4H-1,2,4-triazole (1′d) was isolated as a white solid (>99% ee). [α]_{D}²² +77.4° (c 0.1, MeOH). Peak 2: 3-[(R)-(3-bromophenyl)(cyclobutyl)methyl]-4-methyl-4H-1,2,4-triazole (1′e) was isolated as a white solid (99% ee). [α]_{D}²² −49.6° (c 0.1, MeOH).

### Step 5: 3-{cyclobutyl[3-(4,4,5,5-tetramethyl-1,3,2-dioxaborolan-2-yl)phenyl]methyl}-4-methyl-4H-1,2,4-triazole (Intermediate 1)

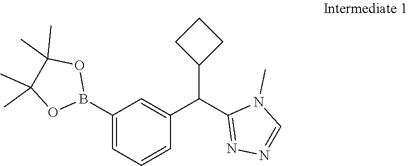

A solution of 3-[(3-bromophenyl)(cyclobutyl)methyl]-4-methyl-4H-1,2,4-triazole (1d) (3.0 g, 9.80 mmol, 1.0 equiv), bis(pinacolato)diboron (2.49 g, 9.80 mmol, 1.0 equiv), potassium acetate (2.88 g, 29.4 mmol, 3.0 equiv), and [1,1′-bis(diphenylphosphino)ferrocene]dichloropalladium(II) (0.503 g, 0.686 mmol, 0.07 equiv) in dioxane (30 mL) was stirred at 90° C. for 16 h. The reaction mixture was cooled and concentrated under reduced pressure. The crude residue was purified by flash column chromatography (ISCO, 40 g SiO₂, DCM/MeOH 0-10%) to give 3-{cyclobutyl[3-(4,4,5,5-tetramethyl-1,3,2-dioxaborolan-2-yl)phenyl]methyl}-4-methyl-4H-1,2,4-triazole (Intermediate 1) (3.27 g, 95%) as a brown solid. LCMS (ESI+) 354.2 (M+H). ¹H NMR (400 MHz, DMSO-d₆) δ 8.31 (s, 1H), 7.55-7.49 (m, 2H), 7.43-7.37 (m, 1H), 7.35-7.28 (m, 1H), 4.19 (d, 1H), 3.37 (s, 3H), 3.15-3.03 (m, 1H), 2.06 (tdd, 1H), 1.85-1.58 (m, 5H), 1.28 (s, 12H).

### Preparation of Intermediate 1′: (R)-3-(cyclobutyl(3-(4,4,5,5-tetramethyl-1,3,2-dioxaborolan-2-yl)phenyl)-methyl)-4-methyl-4H-1,2,4-triazole

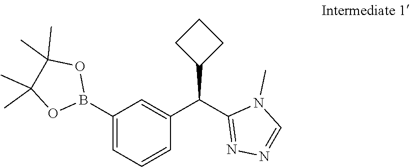

The chiral boronate Intermediate 1′ was made in a similar fashion as detailed above for Intermediate 1 starting from (R)-3-((3-bromophenyl)(cyclobutyl)methyl)-4-methyl-4H-1,2,4-triazole (1′e).

### Preparation of Intermediate 2: 4-methyl-3-({3-[3-(4,4,5,5-tetramethyl-1,3,2-dioxaborolan-2-yl)phenyl]oxetan-3-yl}methyl)-4H-1,2,4-triazole

### Step 1: Ethyl [3-(3-bromophenyl)oxetan-3-yl]acetate (2a)

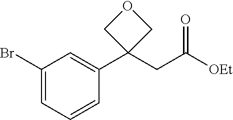 [SMILES: CCOC(=O)CC1(c2cccc(Br)c2)COC1]

To a solution of [Rh(cod)Cl]₂ (4.34 g, 6.33 mmol, 0.05 equiv) in dioxane (600 mL) was added aqueous KOH (153 mL of 1.5 M solution) followed by ethyl 2-(oxetan-3-ylidene)acetate (25 g, 175.9 mmol, 1.0 equiv) and (3-bromophenyl)boronic acid (52.99 g, 263.8 mmol, 1.5 equiv). The reaction was stirred at 25° C. for 16 h. After 16 h, brine (500 mL) was added, the layers were separated, and the aqueous layer was extracted with ethyl acetate (2×). The combined organic layers were washed with brine, dried, filtered, and concentrated under reduced pressure. The crude residue was purified by flash column chromatography (SiO₂, PE/EtOAc 20%) to afford ethyl 2-(3-(3-bromophenyl)oxetan-3-yl)acetate (2a) as yellow oil (38 g, 65%). LCMS (ESI+) 299.0, 301.0 (M+H). ¹H NMR (400 MHz, DMSO-d₆) δ 7.47-7.43 (m, 2H), 7.35-7.25 (m, 2H), 4.77 (dd, 4H), 3.92 (q, 2H), 2.52-2.48 (m, 2H), 1.04 (t, 3H).

### Step 2: 2-[3-(3-bromophenyl)oxetan-3-yl]acetohydrazide (2b)

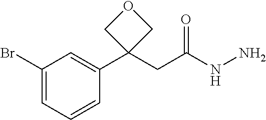 [SMILES: NNC(=O)CC1(c2cccc(Br)c2)COC1]

To a solution of ethyl 2-[3-(3-bromophenyl)oxetan-3-yl]acetate (2a) (38 g, 127 mmol, 1.0 equiv) in EtOH (250 mL) was added hydrazine hydrate (16.26 g, 508 mmol, 80%, 4.0 equiv) dropwise at 25° C. The mixture was stirred at 80° C. for 16 h. Then, additional hydrazine hydrate (16.26 g, 508 mmol, 80%, 4.0 equiv) was added and the mixture was stirred at 80° C. After 16 h, the mixture was concentrated and the crude residue was purified by flash column chromatography (SiO₂, DCM/MeOH 6%) to afford 2-[3-(3-bromophenyl)oxetan-3-yl]acetohydrazide (2b) as a yellow solid (34.8 g, 86%). LCMS (ESI+) 285.0, 287.0 (M+H).

### Step 3: 2-{[3-(3-bromophenyl)oxetan-3-yl]acetyl}-N-methylhydrazine-1-carbothioamide (2c)

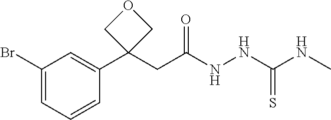 [SMILES: CNC(=S)NNC(=O)CC1(c2cccc(Br)c2)COC1]

To a solution of 2-[3-(3-bromophenyl)oxetan-3-yl]acetohydrazide (2b) (34.8 g, 122 mmol, 1.0 equiv) in THF (260 mL) was added isothiocyanatomethane (12.49 g, 170.7 mmol, 1.4 equiv) and the solution was stirred at 20° C. After 16 h, the mixture was filtrated to afford 2-{[3-(3-bromophenyl)oxetan-3-yl]acetyl}-N-methylhydrazine-1-carbothioamide (2c) as a white solid (34.3 g, 88%). LCMS (ESI+) 358.0, 360.0 (M+H).

### Step 4: 5-{[3-(3-bromophenyl)oxetan-3-yl]methyl}-4-methyl-4H-1,2,4-triazole-3-thiol (2d)

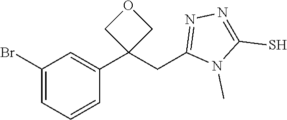 [SMILES: Cn1c(S)nnc1CC1(c2cccc(Br)c2)COC1]

A mixture of 2-{[3-(3-bromophenyl)oxetan-3-yl]acetyl}-N-methylhydrazine-1-carbothioamide (2c) (14.4 g, 40.195 mmol, 1.0 equiv) in sodium hydroxide (670 mL, 670 mmol, 1 M, 7.0 equiv) was stirred at 25° C. After 4 h, the reaction was diluted with water and the pH value of the solution was adjusted to 5 with aq. HCl (1 N). The mixture was extracted with EtOAc (4×30 mL), and the combined organic layers were washed with brine, dried over Na₂SO₄, filtered, and concentrated to afford 5-{[3-(3-bromophenyl)oxetan-3-yl]methyl}-4-methyl-4H-1,2,4-triazole-3-thiol (2d) as a white solid (32 g, 95%). LCMS (ESI+) 340.0, 342.0 (M+H).

### Step 5: 3-{[3-(3-bromophenyl)oxetan-3-yl]methyl}-4-methyl-4H-1,2,4-triazole (2e)

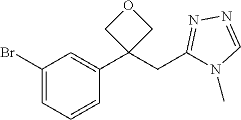 [SMILES: Cn1cnnc1CC1(c2cccc(Br)c2)COC1]

To a solution of 5-{[3-bromophenyl)oxetan-3-yl]methyl}-4-methyl-4H-1,2,4-triazole-3-thiol (2d) (32 g, 94.1 mmol, 1.0 equiv) in water (130 mL) was added sodium nitrite (45.4 g, 658 mmol, 7.0 equiv), followed by the addition of nitric acid (660 mL, 1 M) dropwise with stirring at 0° C. After stirring for 3 h at 25° C., the mixture was basified with saturated aqueous NaHCO₃. The aqueous solution was extracted with DCM (3×300 mL). The combined organic layers were dried over Na₂SO₄, filtered, and concentrated under reduced pressure. The crude residue was slurried with DCM/PE (1/15) to afford 3-{[3-(3-bromophenyl)oxetan-3-yl]methyl}-4-methyl-4H-1,2,4-triazole (2e) as a yellow solid (27 g, 93%). LCMS (ESI+) 308.0, 310.0 (M+H). ¹H NMR (400 MHz, DMSO-d₆) δ 8.23 (s, 1H), 7.43 (ddd, 1H), 7.25-7.21 (m, 2H), 7.02-6.98 (m, 1H), 4.89 (d, 2H), 4.83 (d, 2H), 3.49 (s, 2H), 3.02 (s, 3H).

### Step 6: 4-methyl-3-({3-[3-(4,4,5,5-tetramethyl-1,3,2-dioxaborolan-2-yl)phenyl]oxetan-3-yl}methyl)-4H-1,2,4-triazole (Intermediate 2)

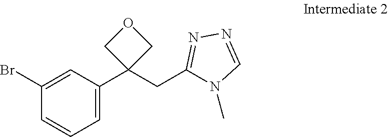 [SMILES: Cn1cnnc1CC1(c2cccc(Br)c2)COC1]

To a solution of compound 3-((3-(3-bromophenyl)oxetan-3-yl)methyl)-4-methyl-4H-1,2,4-triazole (2e) (140 g, 454 mmol, 1 equiv) in dioxane (1.40 L) was added bis(pinacolato)diboron (173 g, 681 mmol, 1.5 equiv), KOAc (134 g, 1.36 mol, 3 equiv), the mixture was purged with N₂ 3 times, then the Pd(dppf)Cl₂·CH₂Cl₂ (18.6 g, 22.7 mmol, 5 mol %) was added into the solution, the mixture was purged with N₂ 3 times and stirred at 90° C. for 16 h. The reaction mixture was filtered through silica gel (100-200, 200 g), and washed with DCM/MeOH (10/1, 2 L). The filtrate was concentrated. The crude product was triturated with PE/EA (8/1, 500 mL) at 25° C. for 2 h. The solid was collected by filtration and dried under reduced pressure to give 4-methyl-3-({3-[3-(4,4,5,5-tetramethyl-1,3,2-dioxaborolan-2-yl)phenyl]oxetan-3-yl}methyl)-4H-1,2,4-triazole (Intermediate 2) (110 g, 68%) as a brown solid. LCMS (ESI+) 356.1 (M+H)⁺.

### Preparation of Intermediate 3: 3-bromo-5-(trifluoromethyl)-1H-pyrazolo[4,3-d]pyrimidine

### Step 1: 4-bromo-2-(trifluoromethyl)pyrimidin-5-amine (3a)

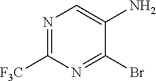 [SMILES: Nc1cnc(C(F)(F)F)nc1Br]

To a solution of 2-(trifluoromethyl)pyrimidin-5-amine (502 mg, 3.08 mmol, 1.0 equiv) in MeCN (8 mL) was added NBS (685 mg, 3.69 mmol, 1.20 equiv) and the reaction was stirred at room temperature. After 24 h, the reaction was quenched with saturated aqueous sodium thiosulfate. The layers were separated, and the aqueous layer was extracted with DCM (2×15 mL). The combined organic layers were dried over Na₂SO₄, filtered, and concentrated under reduced pressure. The crude residue was purified via flash column chromatography (ISCO, SiO₂, heptane/EtOAc 0-100%) to afford 4-bromo-2-(trifluoromethyl)pyrimidin-5-amine (3a) as a dark colored solid (474 mg, 64%). LCMS (ESI+) 266.9 (M+H)⁺. ¹H NMR (400 MHz, CDCl₃) δ 8.20 (s, 1H).

### Step 2: 4-methyl-2-(trifluoromethyl)pyrimidin-5-amine (3b)

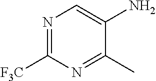

A mixture of 4-bromo-2-(trifluoromethyl)pyrimidin-5-amine (3a) (436 mg, 1.80 mmol, 1.0 equiv), trimethylboroxine (0.501 mL, 3.60 mmol, 2.0 equiv), K₂CO₃ (747 mg, 5.40 mmol, 3.0 equiv), and Pd(dppf)Cl₂·CH₂Cl₂ (147 mg, 0.180 mmol, 10 mol %) in dioxane (4 mL) and water (0.25 mL) was heated to 140° C. in the microwave for 1 h. The reaction mixture was then filtered through celite and washed with DCM. The filtrate was diluted with water (20 mL) and extracted with DCM (2×20 mL). The combined organic layers were dried over Na₂SO₄, filtered, and concentrated under reduced pressure. The crude residue was purified via flash column chromatography (Isco, SiO₂, heptane/EtOAc 0-100%) to afford 4-methyl-2-(trifluoromethyl)pyrimidin-5-amine (3b) as a dark colored solid (222 mg, 70%). LCMS (ESI+) 178.0 (M+H)⁺. ¹H NMR (400 MHz, CDCl₃) δ 8.14 (s, 1H), 2.49 (s, 3H).

### Step 3: 5-(trifluoromethyl)-1H-pyrazolo[4,3-d]pyrimidine (3c)

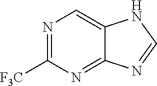 [SMILES: FC(F)(F)c1ncc2[nH]cnc2n1]

To a solution of 4-methyl-2-(trifluoromethyl)pyrimidin-5-amine (3b) (219 mg, 1.24 mmol, 1.0 equiv) in AcOH (2.5 mL) was added sodium nitrite (2.60 mL, 0.5 M, 1.24 mmol, 1.0 equiv) and water (1 mL) and the mixture was stirred at room temperature. After 24 h, the volatiles were removed under reduced pressure and saturated aqueous sodium bicarbonate was added. The aqueous layer was extracted with DCM (2×5 mL). The combined organic layers were dried over Na₂SO₄, filtered, and concentrated under reduced pressure. The crude residue was purified via flash column chromatography (ISCO, SiO₂, heptane/EtOAc 0-100%) to afford 5-(trifluoromethyl)-1H-pyrazolo[4,3-d]pyrimidine (3c) as a yellow solid (40 mg, 17%). LCMS (ESI+) 186.9 (M−H)⁻. ¹H NMR (400 MHz, CDCl₃) δ 9.40 (s, 1H), 8.59 (s, 1H).

### Step 4: 3-bromo-5-(trifluoromethyl)-1H-pyrazolo[4,3-d]pyrimidine (Intermediate 3)

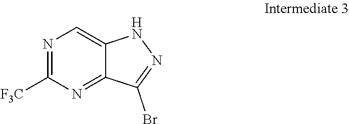 [SMILES: FC(F)(F)c1ncc2[nH]nc(Br)c2n1]

To a solution of 5-(trifluoromethyl)-1H-pyrazolo[4,3-d]pyrimidine (3c) (40 mg, 0.21 mmol, 1.0 equiv) in HFIP (1.06 mL) was added NBS (45.4 mg, 0.255 mmol, 1.20 equiv) and the reaction was stirred at 40° C. After 2 h, the reaction was quenched with saturated aqueous sodium thiosulfate. The layers were separated, and the aqueous layer was extracted with DCM (2×15 mL). The combined organic layers were dried over Na₂SO₄, filtered, and concentrated under reduced pressure. The crude residue was purified via flash column chromatography (ISCO, SiO₂, heptane/EtOAc 0-100%) to afford 3-bromo-5-(trifluoromethyl)-1H-pyrazolo[4,3-d]pyrimidine (Intermediate 3) as a yellow solid (26 mg, 46%). LCMS (ESI+) 264.9 (M−H)⁻.

### Preparation of Intermediate 4: tert-Butyl 3-bromo-5-(trifluoromethyl)-1H-pyrazolo[3,4-b]pyridine-1-carboxylate

### Step 1: 3-bromo-5-(trifluoromethyl)-1H-pyrazolo[3,4-b]pyridine (4a)

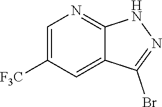 [SMILES: FC(F)(F)c1cnc2[nH]nc(Br)c2c1]

To a solution of 5-(trifluoromethyl)-1H-pyrazolo[3,4-b]pyridine (130 mg, 0.521 mmol, 1.0 equiv) in MeCN (5.0 mL) was added NBS (102 mg, 0.573 mmol, 1.1 equiv) and the reaction was stirred overnight at room temperature. Then the reaction mixture was concentrated under reduced pressure and then purified via flash column chromatography (ISCO, SiO₂, DCM/EtOAc 0-20%) to afford 3-bromo-5-(trifluoromethyl)-1H-pyrazolo[3,4-b]pyridine (4a) as a white solid (125 mg, 90%). ¹H NMR (400 MHz, DMSO-d₆) δ 14.55 (br s, 1H), 8.97 (d, J=1.8 Hz, 1H), 8.62 (s, 1H).

### Step 2: tert-Butyl 3-bromo-5-(trifluoromethyl)-1H-pyrazolo[3,4-b]pyridine-1-carboxylate (Intermediate 4)

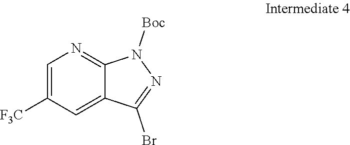 [SMILES: CC(C)(C)OC(=O)n1nc(Br)c2cc(C(F)(F)F)cnc21]

To a solution of 3-bromo-5-(trifluoromethyl)-1H-pyrazolo[3,4-b]pyridine (4a) (125 mg, 0.470 mmol, 1.0 equiv) in MeCN (5.0 mL) was added Boc₂O (118 mg, 0.540 mmol, 1.15 equiv) and Et₃N (78.6 uL, 0.564 mmol, 1.20 equiv) and the reaction was stirred at room temperature. After 16 h, the reaction mixture was concentrated under reduced pressure and purified via flash column chromatography (ISCO, SiO₂, PE/EtOAc 0-13%) to afford tert-butyl 3-bromo-5-(trifluoromethyl)-1H-pyrazolo[3,4-b]pyridine-1-carboxylate (Intermediate 4) as a white solid (115 mg, 67%). LCMS (ESI+) 265.8 (M+H−Boc)⁺. ¹H NMR (400 MHz, DMSO-d₆) δ 9.17 (d, J=1.8 Hz, 1H), 8.72 (d, J=1.4 Hz, 1H), 1.64 (s, 9H).

### Preparation of Intermediate 5: [3-bromo-5-(trifluoromethyl)-1H-pyrazolo[3,4-c]pyridin-7-yl]methanol

### Step 1: [5-(trifluoromethyl)-1H-pyrazolo[3,4-c]pyridin-7-yl]methanol (5a)

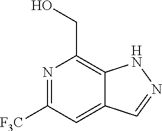 [SMILES: OCc1nc(C(F)(F)F)cc2cn[nH]c12]

A mixture of 5-(trifluoromethyl)-1H-pyrazolo[3,4-c]pyridine (100 mg, 0.53 mmol, 1 equiv), succinic acid (316 mg, 2.67 mmol, 5 equiv), ammonium persulfate (610 mg, 2.67 mmol, 5 equiv) in water (1.8 mL) and ethanol (3.6 mL) was stirred at 70° C. for 2 h. Then the reaction was cooled to room temperature and volatiles were removed under reduced pressure. EtOAc (20 mL) and saturated aqueous NaHCO₃ (8 mL, pH 4) were added, and the layers were separated. The aqueous layer was extracted with EtOAc (2×20 mL).

The combined organic layers were dried over Na₂SO₄, filtered, and concentrated under reduced pressure. The crude residue was purified via flash column chromatography (ISCO, 12 g SiO₂, 0/100% heptane/EtOAc) to afford [5-(trifluoromethyl)-1H-pyrazolo[3,4-c]pyridin-7-yl]methanol (5a) (108 mg, 87%) as a light yellow solid. LCMS (APCI+) 232.0 (M+H). ¹H NMR (400 MHz, DMSO-d₆) δ 13.81 (br s, 1H), 8.36 (s, 1H), 8.22 (s, 1H), 5.88 (br s, 1H), 5.16 (q, J=6.6 Hz, 1H), 1.54 (d, J=6.6 Hz, 3H). ¹⁹F NMR (376 MHz, DMSO-d₆) δ −64.4.

### Step 2: [3-bromo-5-(trifluoromethyl)-1H-pyrazolo[3,4-c]pyridin-7-yl]methanol (Intermediate 5)

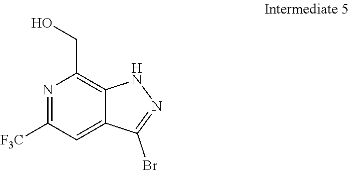 [SMILES: OCc1nc(C(F)(F)F)cc2c(Br)n[nH]c12]

To a solution of [5-(trifluoromethyl)-1H-pyrazolo[3,4-c]pyridin-7-yl]methanol (5a) (140 mg, 0.645 mmol, 1.0 equiv) in HFIP (3.22 mL) was added NBS (172 mg, 0.967 mmol, 1.50 equiv) and the reaction was stirred at 40° C. After 18 h, the reaction was quenched with saturated aqueous sodium thiosulfate. The layers were separated, and the aqueous layer was extracted with DCM (2×15 mL). The combined organic layers were dried over Na₂SO₄, filtered, and concentrated under reduced pressure. The crude residue was purified via flash column chromatography (Isco, SiO₂, heptane/EtOAc 0-100%) to afford 3-bromo-5-(trifluoromethyl)-1H-pyrazolo[3,4-c]pyridin-7-yl]methanol (Intermediate 5) as an off-white solid (130 mg, 68%). LCMS (ESI+) 296.0, 298.0 (M+H)⁺. ¹H NMR (400 MHz, CD₃OD) δ 7.93 (s, 1H), 5.11 (s, 2H).

### Preparation of Intermediate 5′: [3-iodo-5-(trifluoromethyl)-1H-pyrazolo[3,4-c]pyridin-7-yl]methanol

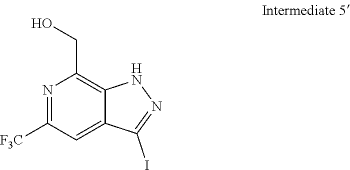 [SMILES: OCc1nc(C(F)(F)F)cc2c(I)n[nH]c12]

To a solution of [5-(trifluoromethyl)-1H-pyrazolo[3,4-c]pyridin-7-yl]methanol (5a) (740 mg, 3.41 mmol, 1.0 equiv) in MeOH (34.1 mL) was added NIS (1150 mg, 5.11 mmol, 1.50 equiv) and the reaction was stirred at room temperature. After 24 h, the reaction was concentrated under reduced pressure, then treated with 10% aqueous sodium thiosulfate. Then EtOAc was added, and the layers were separated, and the aqueous layer was extracted with EtOAc (2×50 mL). The combined organic layers were dried over Na₂SO₄, filtered, and concentrated under reduced pressure. The solid was triturated with DCM, filtered, and washed with DCM to afford 3-iodo-5-(trifluoromethyl)-1H-pyrazolo[3,4-c]pyridin-7-yl]methanol (Intermediate 5′) as a pale yellow solid (752 mg, 64%). LCMS (ESI+) 343.9 (M+H)⁺. ¹H NMR (400 MHz, DMSO-d₆) δ 14.36 (s, 1H), 7.82 (s, 1H), 5.81 (t, J=5.8 Hz, 1H), 4.97 (d, J=5.9 Hz, 2H).

### Preparation of Intermediate 6: 1-[3-bromo-5-(trifluoromethyl)-1H-pyrazolo[3,4-c]pyridin-7-yl]ethan-1-ol

### Step 1: 1-[5-(trifluoromethyl)-1H-pyrazolo[3,4-c]pyridin-7-yl]ethan-1-ol (6a)

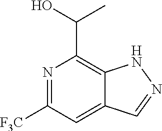 [SMILES: CC(O)c1nc(C(F)(F)F)cc2cn[nH]c12]

A jacketed 5 L Radleys reactor was connected to a nitrogen bubbler line, which was vented to an empty trap, and then to a 0.25 N NaOH aqueous solution to quench the by-product acetaldehyde. To the reactor was added ethanol (450 mL) and solid 5-(trifluoromethyl)-1H-pyrazolo[3,4-c]pyridine (50.4 g, 270 mmol, 1 equiv). Additional ethanol (450 mL) was added, and the mixture was stirred at 200 rpm. Water (450 mL) was then added, and the mixture was heated to 80° C. (internal temperature).

In a separate flask, ammonium persulfate (30.8 g, 135 mmol, 0.5 equiv) was dissolved in water (56 mL) at room temperature. This 0.5 equiv ammonium persulfate solution was added to the reactor at 80° C. using a glass addition funnel over 5-10 minutes at 2 h increments until the total ammonium persulfate used was 3.5 equiv. Internal temperature was stable at 80° C. during each addition. The reaction was then cooled to room temperature and once at internal temperature 20° C., a solution of sodium thiosulfate (Na₂S₂O₃, 21.3 g, 0.5 equiv in 50 mL water) was added to the pot using a syringe over 5 min. The mixture was stirred for 15 min and was at pH 1. Then, an aqueous NaOH solution (5 N, 377 mL, about 7 equiv) was added over 30 min to reach pH 7. During this addition, internal temperature was stable at +16° C. to +18° C.

The volatiles were evaporated (5 mmHg ca. oil pump, 50° C.) to give a volume of about 500 mL. Ethyl acetate (1 L) was added and agitated. The layers were separated, and the aqueous layer was extracted with more ethyl acetate (3×500 mL). The combined organic layer was dried over Na₂SO₄, filtered, and evaporated to give a gum (68 g). LCMS showed about 9:1 mixture of product: ketone side-product (which could be reduced as follows).

The above gum (68 g) was dissolved in ethanol (600 mL), and the solution was cooled in an ice-water bath. NaBH₄ (1.5 g, 0.15 equiv-same equivalent with ketone determined by LCMS) was added. The mixture was stirred under nitrogen in the aged cold bath for about 2 h. Then, acetic acid (7.7 mL, 0.5 equiv) was added, and the mixture was stirred at room temperature overnight to break up product-borane complex. The volatiles were evaporated (5 mmHg ca., 50° C.) to give a gum (74 g). Saturated aqueous NaHCO₃ (200 mL) and ethyl acetate (400 mL) were added and agitated. The layers were separated and extracted with more ethyl acetate (3×250 mL). The combined organic layer was dried over Na₂SO₄ and evaporated to give a light-yellow color solid (69 g).

Ethyl acetate (25 mL) was added to the above solid (69 g) and the mixture was placed onto a pre-heated oil bath at 50° C., to dissolve some solid. To this thick paste/suspension was added heptane (500 mL) to give a suspension. The mixture was stirred and heated to 90° C. (oil bath temperature) under nitrogen for 2 h. The heat was then turned off and stirring continued overnight at room temperature. The solid was filtered, washed with heptane, and dried to give the 1-[5-(trifluoromethyl)-1H-pyrazolo[3,4-c]pyridin-7-yl]ethan-1-ol (6a) (52.5 g, 84%). LCMS (ESI+) 232.0 (M+H). ¹H NMR (400 MHz, CDCl₃) δ 11.47 (br s, 1H), 8.24 (s, 1H), 7.99 (s, 1H), 5.47 (br d, J=6.1 Hz, 1H), 2.90 (br s, 1H), 1.75 (br d, J=6.3 Hz, 3H). ¹⁹F NMR (377 MHz, CDCl₃) δ −66.3.

### Step 2: 1-[3-bromo-5-(trifluoromethyl)-1H-pyrazolo[3,4-c]pyridin-7-yl]ethan-1-ol (Intermediate 6)

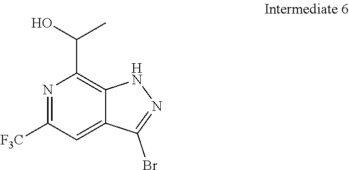 [SMILES: CC(O)c1nc(C(F)(F)F)cc2c(Br)n[nH]c12]

A mixture of 1-[5-(trifluoromethyl)-1H-pyrazolo[3,4-c]pyridin-7-yl]ethan-1-ol (6a) (42.9 g, 185.4 mmol) and NBS (39.6 g, 222 mmol, 1.2 equiv) in acetonitrile (371 mL, 0.5 M) was stirred at room temperature for 2 h. Then, the reaction mixture was quenched with 50% aqueous sodium thiosulfate, and saturated aqueous NaHCO₃ was added. The acetonitrile was evaporated to give a rust colored solid. The mixture was diluted with water then heated to 60° C. with stirring to make sure all the salts went into solution. Heating was stopped and stirring continued at room temperature overnight. The solid was filtered off using a Buchner funnel then air dried. The solid was further dried by azeotroping with toluene. The solid was then suspended in a 9:1 mixture of heptane-ethyl acetate, then filtered off using a Buchner filter. The solid was further dried under house vacuum at 70° C. to give 1-[3-bromo-5-(trifluoromethyl)-1H-pyrazolo[3,4-c]pyridin-7-yl]ethan-1-ol (Intermediate 6) as a tan solid (41 g, 71%). The mother liquor was concentrated and purified by ISCO-Rf-120 g column eluting with 0-50% ethyl acetate-heptane to give an additional crop of 1-[3-bromo-5-(trifluoromethyl)-1H-pyrazolo[3,4-c]pyridin-7-yl]ethan-1-ol (5.1 g, 9%; total 80%). LCMS (ESI+) 310.0/312.0 (M+H). ¹H NMR (400 MHz, DMSO-d₆) δ 14.18 (br s, 1H) 7.97 (s, 1H) 5.97 (br d, J=4.00 Hz, 1H) 5.22-5.11 (m, 1H) 1.54 (d, J=6.63 Hz, 3H). ¹⁹F NMR (377 MHz, DMSO-d₆) δ −64.7.

### Preparation of Intermediate 6′: 1-[3-iodo-5-(trifluoromethyl)-1H-pyrazolo[3,4-c]pyridin-7-yl]ethan-1-ol

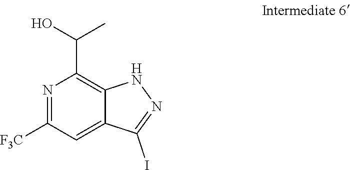 [SMILES: CC(O)c1nc(C(F)(F)F)cc2c(I)n[nH]c12]

A mixture of 1-[5-(trifluoromethyl)-1H-pyrazolo[3,4-c]pyridin-7-yl]ethan-1-ol (6a) (1.83 g, 7.89 mmol, 1.0 equiv) and NIS (2.72 g, 11.8 mmol, 1.5 equiv) in acetonitrile (26.3 mL, 0.3 M) was stirred at 60° C. for 24 h. Then, the reaction mixture was concentrated under reduced pressure. After dilution with EtOAc, the organic layer was washed with 10% aqueous sodium thiosulfate and brine. The combined organic layers were dried over Na₂SO₄, filtered, and concentrated under reduced pressure. Water was added to the residue and stirred at room temperature for 10 minutes. The solid was filtered, washed with water, and dried to afford 1-[3-iodo-5-(trifluoromethyl)-1H-pyrazolo[3,4-c]pyridin-7-yl]ethan-1-ol (Intermediate 6′) as a yellow solid (2.47 g, 88%). LCMS (ESI+) 357.9 (M+H)⁺. ¹H NMR (400 MHz, DMSO-d₆) δ 14.37-14.11 (m, 1H), 7.80 (s, 1H), 5.96 (d, J=4.4 Hz, 1H), 5.27-5.03 (m, 1H), 1.53 (d, J=6.6 Hz, 3H).

### Preparation of Intermediate 6′a: 1-[3-bromo-5-(trifluoromethyl)-1H-pyrazolo[3,4-c]pyridin-7-yl]ethan-1-one

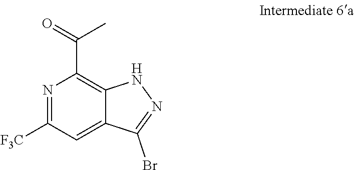 [SMILES: CC(=O)c1nc(C(F)(F)F)cc2c(Br)n[nH]c12]

Manganese dioxide (450 mg, 5.18 mmol) was added to a solution of 1-[3-bromo-5-(trifluoromethyl)-1H-pyrazolo[3,4-c]pyridin-7-yl]ethan-1-ol (Intermediate 6) (231 mg, 0.745 mmol) in EtOAc (10 mL). The mixture was heated at 60° C. for 4 h and then cooled to room temperature, filtered through Celite, and washed with DCM. The filtrate was concentrated and dried to give 1-[3-bromo-5-(trifluoromethyl)-1H-pyrazolo[3,4-c]pyridin-7-yl]ethan-1-one (Intermediate 6′a) as a pale-yellow solid (209 mg, 91%). ¹H NMR (400 MHz, CDCl₃) δ 11.81 (br s, 1H), 8.30-8.14 (m, 1H), 2.91 (s, 3H).

### Preparation of Intermediate 7: 3-{3-[(R)-cyclobutyl(4-methyl-4H-1,2,4-triazol-3-yl)methyl]phenyl}-5-(trifluoromethyl)-1H-pyrazolo[3,4-c]pyridine-7-carbaldehyde

### Step 1: [3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]methanol (7a)

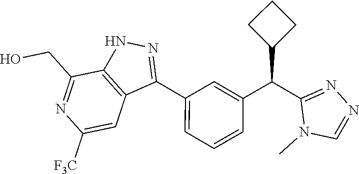 [SMILES: Cn1cnnc1[C@@H](c1cccc(-c2n[nH]c3c(CO)nc(C(F)(F)F)cc23)c1)C1CCC1]

A mixture of 3-iodo-5-(trifluoromethyl)-1H-pyrazolo[3,4-c]pyridin-7-yl)methanol (Intermediate 5′) (262 mg, 0.764 mmol), (R)-3-(cyclobutyl(3-(4,4,5,5-tetramethyl-1,3,2-dioxaborolan-2-yl)phenyl)methyl)-4-methyl-4H-1,2,4-triazole (Intermediate 1′) (297 mg, 0.841 mmol) in DME/IPA (2/1) (6 mL, c=0.1 M) was added aqueous sodium bicarbonate (257 mg, 3.06 mmol, 3 mL, 1.0 M) and Pd(PPh₃)₄ (88.3 mg, 0.0764 mmol). The mixture was heated at 120° C. overnight and then cooled to room temperature, diluted with water (10 mL) and extracted with DCM (2×10 mL). The combined organic layers were dried over sodium sulfate and concentrated. The residue was purified by flash column chromatography (ISCO, solvent 0-10% MeOH/1:1 DCM/EtOAc to 10% MeOH/DCM) to give [3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]methanol (7a) as a pale yellow foam (289 mg, 86%). LCMS (APCI+) 443.0. ¹H NMR (400 MHz, CDCl₃) δ 8.20-8.15 (m, 1H), 8.11 (s, 1H), 7.93 (s, 1H), 7.81 (d, J=7.8 Hz, 1H), 7.58-7.47 (m, 1H), 7.36 (d, J=7.7 Hz, 1H), 5.36 (d, J=2.7 Hz, 2H), 4.06 (d, J=10.8 Hz, 1H), 3.56 (s, 3H), 3.54-3.40 (m, 1H), 2.38-2.32 (m, 1H), 2.03-1.97 (m, 2H), 1.94-1.88 (m, 2H), 1.80-1.65 (m, 2H).

### Step 2: 3-{3-[(R)-cyclobutyl(4-methyl-4H-1,2,4-triazol-3-yl)methyl]phenyl}-5-(trifluoromethyl)-1H-pyrazolo[3,4-c]pyridine-7-carbaldehyde (Intermediate 7)

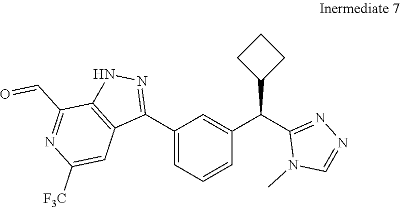 [SMILES: Cn1cnnc1[C@@H](c1cccc(-c2n[nH]c3c(C=O)nc(C(F)(F)F)cc23)c1)C1CCC1]

To a solution of [3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methanol (7a) (253 mg, 0.572 mmol) in CHCl₃ (10.0 mL) was added MnO₂ (1.49 g, 17.2 mmol). The resulting mixture was heated at 70° C. for 20 hr. The mixture was cooled to RT, filtered through Celite, and washed with CH₂Cl₂. The filtrate was concentrated and purified by flash column chromatography (ISCO, 12 g SiO₂, solvent 0-10% MeOH/DCM to give 3-{3-[(R)-cyclobutyl(4-methyl-4H-1,2,4-triazol-3-yl)methyl]phenyl}-5-(trifluoromethyl)-1H-pyrazolo[3,4-c]pyridine-7-carbaldehyde (Intermediate 7) as a yellow color foam (166 mg, 66%). ¹H NMR (400 MHz, CDCl₃) δ 10.37 (s, 1H), 8.49 (s, 1H), 8.12 (s, 1H), 7.90-7.88 (m, 1H), 7.86-7.82 (m, 1H), 7.54 (t, J=7.7 Hz, 1H), 7.38 (d, J=7.8 Hz, 1H), 4.05 (d, J=10.5 Hz, 1H), 3.48 (s, 3H), 3.44 (br. dd, J=7.8, 2.8 Hz, 1H), 2.04-1.92 (m, 4H), 1.80-1.69 (m, 2H). m/z (APCI+) for (C₂₂H₁₉F₃N₆O) 441.1 (M+H).

### Preparation of Intermediate 8: 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-one

### Step 1: 1-[3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]ethan-1-ol (8a)

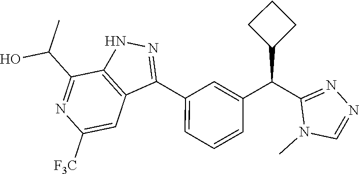 [SMILES: CC(O)c1nc(C(F)(F)F)cc2c(-c3cccc([C@H](c4nncn4C)C4CCC4)c3)n[nH]c12]

To a solution of 1-(3-iodo-5-(trifluoromethyl)-1H-pyrazolo[3,4-c]pyridin-7-yl)ethan-1-ol (Intermediate 6′) (473 mg, 1.32 mmol) and (R)-3-(cyclobutyl(3-(4,4,5,5-tetramethyl-1,3,2-dioxaborolan-2-yl)phenyl)methyl)-4-methyl-4H-1,2,4-triazole (Intermediate 1′) (562.0 mg, 1.59 mmol) in iso-propanol (8.83 mL, c=0.15 M) was added aqueous potassium phosphate (703 mg, 3.31 mmol, 3.31 mL, 1.0 M) and cataCXium® A Pd G3 (96.5 mg, 0.132 mmol). The mixture was degassed three times and heated at 110° C. overnight. The mixture was cooled to room temperature, diluted with brine (10 mL) and extracted with DCM (2×10 mL). The organic layers were dried over sodium sulfate, filtered, and concentrated. The residue was purified by flash column chromatography (ISCO, solvent 0-10% MeOH/1:1 DCM/EtOAc) to give 1-[3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]ethan-1-ol (8a) as an off-white solid (421 mg, 70%). LCMS (APCI+) 457.0. ¹H NMR (400 MHz, CDCl₃) δ 8.12 (d, J=5.3 Hz, 1H), 8.05 (d, J=2.4 Hz, 1H), 8.00-7.93 (m, 1H), 7.82 (br d, J=7.6 Hz, 1H), 7.51 (dt, J=1.3, 7.6 Hz, 1H), 7.39-7.31 (m, 1H), 5.61 (qd, J=6.5, 10.5 Hz, 1H), 4.02 (dd, J=4.6, 10.8 Hz, 1H), 3.53 (d, J=6.5 Hz, 4H), 2.43-2.28 (m, 1H), 2.04-1.80 (m, 8H), 1.79-1.70 (m, 1H).

### Step 2: 1-[3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]ethan-1-one (Intermediate 8)

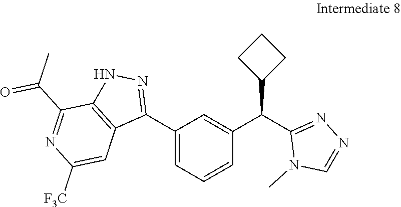 [SMILES: CC(=O)c1nc(C(F)(F)F)cc2c(-c3cccc([C@H](c4nncn4C)C4CCC4)c3)n[nH]c12]

To a solution of 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-ol (8a) (525 mg, 1.15 mmol) in CHCl₃ (14.4 mL, c=0.08 M) was added MnO₂ (2.5 g, 28.7 mmol). The resulting mixture was heated at 70° C. for 24 h. The mixture was cooled to RT, filtered through Celite, and washed with CH₂Cl₂. The filtrate was concentrated and purified by flash column chromatography (ISCO, 12 g SiO₂, solvent 0-10% MeOH/DCM to give 1-[3-{3-[(R)-cyclobutyl(4-methyl-4H-1,2,4-triazol-3-yl)methyl]phenyl}-5-(trifluoro-methyl)-1H-pyrazolo[3,4-c]pyridin-7-yl]ethan-1-one (Intermediate 8) as yellow color solid (397 mg, 76%). ¹H NMR (400 MHz, CDCl₃) δ 11.92 (br. s, 1H), 8.45 (s, 1H), 8.06 (s, 1H), 7.88 (t, J=1.6 Hz, 1H), 7.83 (td, J=1.3, 7.7 Hz, 1H), 7.53 (t, J=7.7 Hz, 1H), 7.41-7.32 (m, 1H), 4.04 (d, J=10.5 Hz, 1H), 3.52-3.37 (m, 4H), 2.93 (s, 3H), 2.49-2.32 (m, 1H), 2.02-1.91 (m, 2H), 1.91-1.83 (m, 2H), 1.82-1.70 (m, 1H); m/z (APCI+) for (C₂₃H₂₁F₃N₆O) 455.1 (M+H).

### Preparation of Intermediate 9: 3-(3-{3-[(4-methyl-4H-1,2,4-triazol-3-yl)methyl]oxetan-3-yl}phenyl)-5-(trifluoromethyl)-1H-pyrazolo[3,4-c]pyridine-7-carbaldehyde

### Step 1: [3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]methanol (9a)

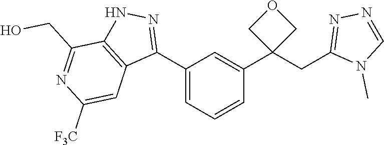 [SMILES: Cn1cnnc1CC1(c2cccc(-c3n[nH]c4c(CO)nc(C(F)(F)F)cc34)c2)COC1]

A mixture of 4-methyl-3-((3-(3-(4,4,5,5-tetramethyl-1,3,2-dioxaborolan-2-yl)phenyl)oxetan-3-yl)methyl)-4H-1,2,4-triazole (Intermediate 2) (110 mg, 0.311 mmol), (3-iodo-5-(trifluoromethyl)-1H-pyrazolo[3,4-c]pyridin-7-yl)methanol (Intermediate 5′) (82 mg, 0.24 mmol), Pd(PPh₃)₄ (27.6 mg, 0.0239 mmol) and aqueous sodium bicarbonate (100 mg, 1.20 mmol, 1.16 mL, 1.03 M) in a mixture of DME/IPA (2:1) (3.0 mL, c=0.08 M) was sparged with N₂ for 5 minutes then heated to 120° C. in a microwave reactor for 120 min. The reaction mixture was diluted with EtOAc and water. The EtOAc layer was washed with brine, dried with sodium sulfate, filtered, and concentrated to an oil. The crude reaction mixture was purified by flash column chromatography (ISCO, 12 g column eluting with 0-100% MeOH-DCM) to give [3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]methanol (9a) (55 mg, 52%). ¹H NMR (400 MHz, DMSO-d₆) δ 8.22 (s, 1H), 8.16 (s, 1H), 7.89 (d, J=7.8 Hz, 1H), 7.59 (s, 1H), 7.47 (t, J=7.6 Hz, 1H), 7.06 (d, J=8.0 Hz, 1H), 5.03-4.92 (m, 6H), 3.57 (s, 2H), 3.15-3.10 (m, 1H), 2.96 (s, 3H).

### Step 2: 3-(3-{3-[(4-methyl-4H-1,2,4-triazol-3-yl)methyl]oxetan-3-yl}phenyl)-5-(trifluoro-methyl)-1H-pyrazolo[3,4-c]pyridine-7-carbaldehyde (Intermediate 9)

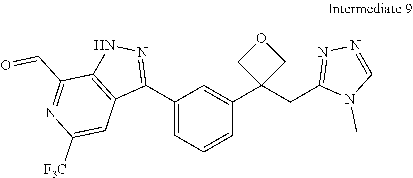 [SMILES: Cn1cnnc1CC1(c2cccc(-c3n[nH]c4c(C=O)nc(C(F)(F)F)cc34)c2)COC1]

To a suspension of [3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]methanol (9a) (1000 mg 2.250 mmol) in CHCl₃ (30.0 mL) and 1,4-dioxane (15.0 mL) was added MnO₂ (1960 mg 22.5 mmol). The suspension was stirred at reflux for 16 h. The mixture was then filtered, and the filter cake was rinsed with DCM (30 mL). The filtrate was concentrated in vacuo to afford 3-(3-{3-[(4-methyl-4H-1,2,4-triazol-3-yl)methyl]oxetan-3-yl}phenyl)-5-(trifluoro-methyl)-1H-pyrazolo[3,4-c]pyridine-7-carbaldehyde (Intermediate 9) (700 mg, 70%) as a yellow solid. ¹H NMR (400 MHz, DMSO-d₆) δ 14.70 (s, 1H), 10.24 (s, 1H), 8.70 (s, 1H), 8.19 (s, 1H), 7.96 (d, J=8.0 Hz, 1H), 7.69 (s, 1H), 7.51 (t, J=7.8 Hz, 1H), 7.13 (d, J=8.3 Hz, 1H), 5.04-4.93 (m, 4H), 3.60 (s, 2H), 3.01 (s, 3H).

### Preparation of Intermediate 10: 1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethan-1-one

### Step 1: 1-[3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]ethan-1-ol (10a)

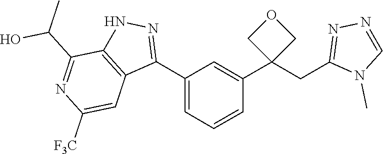 [SMILES: CC(O)c1nc(C(F)(F)F)cc2c(-c3cccc(C4(Cc5nncn5C)COC4)c3)n[nH]c12]

A mixture of 4,4,4′,4′,5,5,5′,5′-octamethyl-2,2′-bi(1,3,2-dioxaborolane (118 mg, 0.466 mmol, 1.5 equiv), 3-{[3-(3-bromophenyl)oxetan-3-yl]methyl}-4-methyl-4H-1,2,4-triazole (2e) (100.0 mg, 0.31 mmol, 1 equiv)_and potassium acetate (91.4 mg, 0.931 mmol, 3 equiv) in 1,4-dioxane (1.6 mL) was deoxygenated with a N₂ bubbler for a few minutes before the addition of 1,1′-bis(diphenylphosphino)ferrocene-palladium(II)dichloride dichloromethane complex (38 mg, 0.0466 mmol, 15 mol %). The mixture was capped and heated at 120° C. for 40 min. The mixture was then cooled to room temperature and then filtered, washed with dichloromethane and the crude obtained after concentration of the filtrate was purified by flash column chromatography (ISCO, DCM/MeOH 0-10%) to give 83 mg (75%) of 4-methyl-3-({3-[3-(4,4,5,5-tetramethyl-1,3,2-dioxaborolan-2-yl)phenyl]oxetan-3-yl}methyl)-4H-1,2,4-triazole (Intermediate 2) as a brown oil.

A mixture of Intermediate 2 (83 mg, 0.233 mmol), 1-(3-bromo-5-(trifluoromethyl)-1H-pyrazolo[3,4-c]pyridin-7-yl)ethan-1-ol (Intermediate 6) (72.2 mg, 0.233 mmol), potassium carbonate (90.1 mg, 0.652 mmol) in 1,4-dioxane (2 mL) and water (0.5 mL) was degassed with nitrogen three times. 1,1′-bis(diphenylphosphino)ferrocene-palladium(II)dichloride dichloromethane complex (50.7 mg, 0.0621 mmol, 20 mol %) was then added and the mixture was heated at 120° C. for one hour. The mixture was then filtered through Celite, and the filtrate was concentrated. The residue was purified by flash column chromatography (ISCO, DCM/MeOH 0-10%) to yield 35 mg (33%) of 1-[3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]ethan-1-ol (10a) as a dark brown solid. LCMS (APCI+) 459.1. ¹H NMR (400 MHz, CDCl₃) δ 8.18-8.04 (m, 1H), 7.95 (s, 1H), 7.81 (br d, J=7.7 Hz, 1H), 7.48 (t, J=7.7 Hz, 1H), 7.42 (s, 1H), 6.94 (br d, J=7.7 Hz, 1H), 5.47 (br d, J=6.2 Hz, 1H), 5.16-5.13 (m, 4H), 3.64 (s, 2H), 2.84 (s, 3H), 1.74 (br d, J=6.4 Hz, 3H).

### Step 2: 1-[3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]ethan-1-one (Intermediate 10)

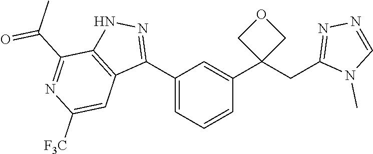 [SMILES: CC(=O)c1nc(C(F)(F)F)cc2c(-c3cccc(C4(Cc5nncn5C)COC4)c3)n[nH]c12]

To a suspension of 1-(3-bromo-5-(trifluoromethyl)-1H-pyrazolo[3,4-c]pyridin-7-yl)ethan-1-one (Intermediate 6′a) (319 mg, 1.04 mmol) and 4-methyl-3-((3-(3-(4,4,5,5-tetramethyl-1,3,2-dioxaborolan-2-yl)phenyl)oxetan-3-yl)methyl)-4H-1,2,4-triazole (Intermediate 2) (441 mg, 1.24 mmol) in dioxane (5.18 mL, c=0.2 M) and water (1870 mg, 104 mmol, 1.87 mL) in a sealable tube, was added sodium carbonate (329 mg, 3.11 mmol) and [1,1′-Bis(diphenylphosphino)ferrocene]dichloropalladium(II) (75.8 mg, 0.104 mmol) and the reaction was bubbled with nitrogen via needle for 5 minutes. The vial was then clamped, and the reaction was stirred at 100° C. for 16 hours. After this time, the mixture was diluted with ethyl acetate (5 mL) and washed with water (5 mL). The aqueous layer was further extracted with ethyl acetate (5 mL×2). The combined organic layers were washed with brine (5 mL), dried over sodium sulfate, and concentrated in vacuo to afford crude product as brown gum. This crude mixture was purified by flash chromatography (ISCO, 0-30% MeOH/DCM) to provide 1-[3-(3-{3-[(4-methyl-4H-1,2,4-triazol-3-yl)methyl]oxetan-3-yl}phenyl)-5-(trifluoro-methyl)-1H-pyrazolo[3,4-c]pyridin-7-yl]ethan-1-one (Intermediate 10) as a beige solid (352 mg, 75%). ¹H NMR (400 MHz, CD₃OD) δ 8.42 (s, 1H), 8.05 (s, 1H), 7.83 (d, J=7.82 Hz, 1H), 7.47-7.43 (m, 1H), 7.42-7.40 (m, 1H), 6.93 (br d, J=7.95 Hz, 1H), 5.06-4.95 (m, 4H), 3.59 (s, 2H), 2.80 (s, 3H), 2.74 (s, 3H). LCMS (ESI+) for (C₂₂H₁₉F₃N₆O₂) 457.10 (M+H).

### Preparation of Intermediate 11: 7-(bromomethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine

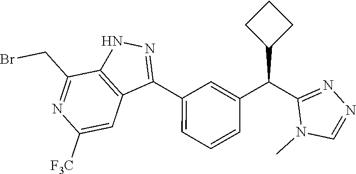 [SMILES: Cn1cnnc1[C@@H](c1cccc(-c2n[nH]c3c(CBr)nc(C(F)(F)F)cc23)c1)C1CCC1]

Triphenylphosphine (246 mg, 0.937 mmol) and carbon tetrabromide (311 mg, 0.937 mmol) was added to a solution of (R)-(3-(3-(cyclobutyl(4-methyl-4H-1,2,4-triazol-3-yl)methyl)phenyl)-5-(trifluoromethyl)-1H-pyrazolo[3,4-c]pyridin-7-yl)methanol (7a) (319 mg, 0.721 mmol) in DCM (7.2 mL, c=0.1 M). The mixture was stirred at room temperature for 3 hours. The volatiles were removed under reduced pressure. The residue was purified by flash column chromatography (ISCO, 12 g SiO₂, solvent 0-10% MeOH in 1:1 EtOAc/DCM) to give 7-(bromomethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 11) as an off-white foam (278 mg, 76%). LCMS (APCI+) for (C₂₂H₂₀BrF₃N₆) 506.9 (M+H).

### Preparation of Intermediate 12: 7-(1-bromoethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine

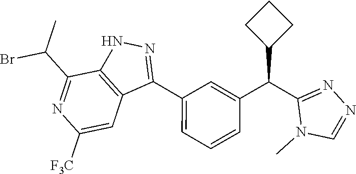 [SMILES: CC(Br)c1nc(C(F)(F)F)cc2c(-c3cccc([C@H](c4nncn4C)C4CCC4)c3)n[nH]c12]

Triphenylphosphine (406 mg, 1.55 mmol) and carbon tetrabromide (513 mg, 1.55 mmol) was added to a solution of 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-ol (8a) (543 mg, 1.19 mmol) in DCM (11.9 mL, c=0.1 M). The mixture was stirred at RT for 2 hr. The volatiles were removed under reduced pressure. The residue was purified by flash column chromatography (ISCO, 12 g SiO₂, solvent, 0-10% MeOH in 1:1 EtOAc/DCM) to give 7-(1-bromoethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 12) as an off-white foam (479 mg, 77%). ¹H NMR (MeOH-d₄, 400 MHz) δ 8.38 (s, 1H), 8.23 (d, J=3.5 Hz, 1H), 7.90 (d, J=7.7 Hz, 1H), 7.9-7.8 (m, 1H), 7.56 (t, 1H, J=7.8 Hz), 7.38 (d, 1H, J=7.7 Hz), 5.85 (q, 1H, J=6.7 Hz), 4.35 (d, 1H, J=10.9 Hz), 3.55 (s, 3H), 3.4-3.3 (m, 1H), 2.4-2.3 (m, 1H), 2.27 (d, 3H, J=6.7 Hz), 2.0-1.9 (m, 4H), 1.9-1.8 (m, 1H); m/z (APCI+) for (C₂₃H₂₂BrF₃N₆) 520.9 (M+H).

### Preparation of Intermediate 13: 7-(bromomethyl)-3-(3-{3-[(4-methyl-4H-1,2,4-triazol-3-yl)methyl]oxetan-3-yl}phenyl)-5-(trifluoromethyl)-1H-pyrazolo[3,4-c]pyridine

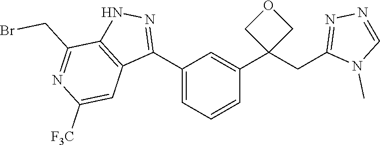 [SMILES: Cn1cnnc1CC1(c2cccc(-c3n[nH]c4c(CBr)nc(C(F)(F)F)cc34)c2)COC1]

To a suspension of (3-(3-(3-((4-methyl-4H-1,2,4-triazol-3-yl)methyl)oxetan-3-yl)phenyl)-5-(trifluoromethyl)-1H-pyrazolo[3,4-c]pyridin-7-yl)methanol (9a) (25 mg, 0.056 mmol) and carbon tetrabromide (26.1 mg, 0.0788 mmol) in DCM (0.5 mL, c=0.1 M) cooled in an ice water bath was added triphenylphosphine (20.7 mg, 0.0788 mmol). After 2 hours, the heterogenous reaction mixture was loaded directly onto a 5 g solid load cartridge and then purified by ISCO-Rf-4 g Gold column eluting with 0-100% MeOH-DCM to yield 23 mg of a mixture containing 7-(bromomethyl)-3-(3-{3-[(4-methyl-4H-1,2,4-triazol-3-yl)methyl]oxetan-3-yl}phenyl)-5-(trifluoromethyl)-1H-pyrazolo[3,4-c]pyridine (Intermediate 13) and an unknown impurity. LCMS-ESI (+): 507/509 (M+H). This material was used as is in subsequent steps.

### Preparation of Intermediate 14: 7-(1-bromoethyl)-3-(3-{3-[(4-methyl-4H-1,2,4-triazol-3-yl)methyl]oxetan-3-yl}phenyl)-5-(trifluoromethyl)-1H-pyrazolo[3,4-c]pyridine

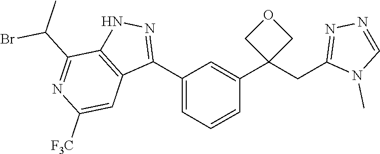 [SMILES: CC(Br)c1nc(C(F)(F)F)cc2c(-c3cccc(C4(Cc5nncn5C)COC4)c3)n[nH]c12]

A 25 mL flask under nitrogen was charged with 1-(3-(3-(3-((4-methyl-4H-1,2,4-triazol-3-yl)methyl)oxetan-3-yl)phenyl)-5-(trifluoromethyl)-1H-pyrazolo[3,4-c]pyridin-7-yl)ethan-1-ol (10a) (204.0 mg, 0.445 mmol) and methylene chloride (2.22 mL, c=0.2 M) and stirred until dissolved, to this solution, were added triphenyl phosphine (175 mg, 0.667 mmol) and carbon tetrabromide (221 mg, 0.667 mmol) and the reaction was stirred at room temperature for 16 hours. After this time, solvents were removed under reduced pressure and the crude material was used in subsequent reactions. m/z (APCI+) for (C₂₂H₂₀BrF₃N₆O), 521.00 (M+H).

### Preparation of Intermediate 15: (R)-6-chloro-3-(3-(cyclobutyl(4-methyl-4H-1,2,4-triazol-3-yl)methyl)phenyl)-5-(trifluoromethyl)-1H-pyrazolo[3,4-b]pyridine

### Step 1: 5-(trifluoromethyl)-1H-pyrazolo[3,4-b]pyridine (15a)

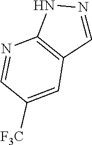 [SMILES: FC(F)(F)c1cnc2[nH]ncc2c1]

To 2-chloro-5-(trifluoromethyl)nicotinaldehyde in EtOH (2 mL, c=2 M) in a 40 mL vial was added hydrazine (916 mg, 28.6 mmol, 0.887 mL) and 4-methylbenzenesulfonic acid hydrate (410 mg, 2.38 mmol). The vial was capped and heated at 130° C. overnight. The mixture was diluted with H₂O (40 mL) and extracted with EtOAc (2×40 mL). The combined organic layers were dried over Na₂SO₄ and concentrated. The crude residue was purified by flash column chromatography (ISCO, solvent 0-100% EtOAc/heptane) to give 5-(trifluoromethyl)-1H-pyrazolo[3,4-b]pyridine (15a) as a white solid (765 mg, 86%). ¹H NMR (400 MHz, CDCl₃) δ=8.90 (d, J=1.7 Hz, 1H), 8.47 (d, J=1.1 Hz, 1H), 8.27 (s, 1H).

### Step 2: 6-chloro-5-(trifluoromethyl)-1H-pyrazolo[3,4-b]pyridine (15b)

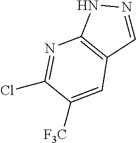 [SMILES: FC(F)(F)c1cc2cn[nH]c2nc1Cl]

To a suspension of 5-(trifluoromethyl)-1H-pyrazolo[3,4-b]pyridine (15a) (414 mg, 2.21 mmol) in DCM (5 mL) in a 40 mL vial was added urea hydrogen peroxide adduct (416 mg, 4.42 mmol) and methyltrioxorhenium (VII) (44.1 mg, 0.177 mmol). The vial was capped, and the mixture was stirred at room temperature overnight. The mixture was then diluted with DCM (20 mL) and transferred to a 100 mL vial and molecular sieves (4 Å, 1.24 g) was added. The mixture was stirred at room temperature for 5 min followed by the addition of tetra-butylammonium chloride (922 mg, 3.32 mmol) and tosic anhydride (1080 mg, 3.32 mmol). The mixture was stirred at room temperature overnight. The mixture was filtered through Celite and washed with DCM and H₂O. The filtrate was dried over sodium sulfate and concentrated to a residue which was purified by flash column chromatography (ISCO, solvent 0-100% EtOAc/heptane) to give 6-chloro-5-(trifluoromethyl)-1H-pyrazolo[3,4-b]pyridine (15b) as a white solid (245 mg, 50%, 12:1 regioisomeric mixture of C7:C5 chloro). m/z (APCI−) for (C₇H₃ClF₃N₃) 219.9 (M−H). ¹H NMR (400 MHz, CDCl₃) δ=8.52 (s, 1H), 8.26 (s, 1H), 8.24 (s, 1H).

### Step 3: tert-butyl 6-chloro-3-iodo-5-(trifluoromethyl)-1H-pyrazolo[3,4-b]pyridine-1-carboxylate (15c)

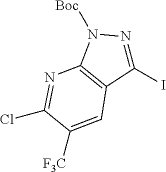 [SMILES: CC(C)(C)OC(=O)n1nc(I)c2cc(C(F)(F)F)c(Cl)nc21]

N-Iodosuccinimide (372 mg, 1.65 mmol) was added to a solution of 6-chloro-5-(trifluoromethyl)-1H-pyrazolo[3,4-b]pyridine (15b) (244.0 mg, 1.10 mmol) in acetonitrile (5.0 mL, c=0.08 M). The mixture was stirred at 100° C. for 5 hours and then treated with 10% sodium thiosulfate and extracted with EtOAc (2×100 mL). The combined organic layers were dried over Na₂SO₄ and concentrated to give a yellow color solid. The above solid was dissolved in DCM (8.0 mL, c=0.08 M), and Boc₂O (288 mg, 1.32 mmol) and triethylamine (223 mg, 2.20 mmol, 0.307 mL) were added. The mixture was stirred at room temperature for 2 hours. The mixture was diluted with H₂O and extracted with DCM (2×5 mL). The combined organic layers were dried over Na₂SO₄ and concentrated. The residue was purified by flash column chromatography (ISCO, solvent 0-100% EtOAc/heptane) to give tert-butyl 6-chloro-3-iodo-5-(trifluoromethyl)-1H-pyrazolo[3,4-b]pyridine-1-carboxylate (15c) as a white solid (428 mg, 87%). m/z (APCI−) for (C₁₂H₁₀ClF₃IN₂O₂) 345.8 (M−H); ¹H NMR (400 MHz, CDCl₃) δ 8.18 (s, 1H), 1.79-1.70 (m, 9H).

### Step 4: (R)-6-chloro-3-(3-(cyclobutyl(4-methyl-4H-1,2,4-triazol-3-yl)methyl)phenyl)-5-(trifluoromethyl)-1H-pyrazolo[3,4-b]pyridine (Intermediate 15)

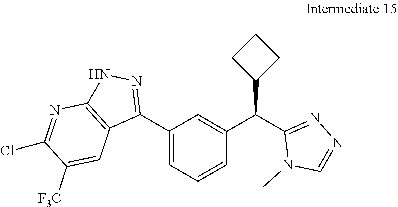 [SMILES: Cn1cnnc1[C@@H](c1cccc(-c2n[nH]c3nc(Cl)c(C(F)(F)F)cc23)c1)C1CCC1]

A mixture of tert-butyl 6-chloro-3-iodo-5-(trifluoromethyl)-1H-pyrazolo[3,4-b]pyridine-1-carboxylate (15c) (354 mg, 0.791 mmol), (R)-3-(cyclobutyl(3-(4,4,5,5-tetramethyl-1,3,2-dioxaborolan-2-yl)phenyl)methyl)-4-methyl-4H-1,2,4-triazole (Intermediate 1′) (233 mg, 0.660 mmol), cesium fluoride (301 mg, 1.98 mmol) and [1,1-bis(diphenylphosphino)ferrocene]-palladium(II) dichloride dichloromethane adduct (135 mg, 0.165 mmol) in 1,4-dioxane (6 mL, c=0.09 M) and water (1.2 mL, c=0.09 M) was degassed thrice with nitrogen. The mixture was heated at 100° C. overnight. The mixture was filtered through Celite and washed with DCM (10 mL). The filtrate was washed with brine (10 mL), dried over Na₂SO₄ and concentrated. The residue was purified by flash column chromatography (ISCO, solvent 0-10% MeOH in DCM) to give (R)-6-chloro-3-(3-(cyclobutyl(4-methyl-4H-1,2,4-triazol-3-yl)methyl)phenyl)-5-(trifluoromethyl)-1H-pyrazolo[3,4-b]pyridine (Intermediate 15) as a brown colored gum (153 mg, 52%). m/z (APCI+) for (C₂₁H₁₈ClF₃N₆) 447.0 (M+H).

### Preparation of Intermediate 16: (S)—N-((3-bromo-5-(trifluoromethyl)-1H-pyrazolo[3,4-c]pyridin-7-yl)methyl)-3,3-difluorocyclopentan-1-amine

### Step 1: 3-bromo-5-(trifluoromethyl)-1H-pyrazolo[3,4-c]pyridine-7-carbaldehyde (16a)

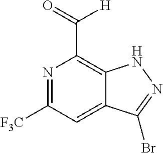 [SMILES: O=Cc1nc(C(F)(F)F)cc2c(Br)n[nH]c12]

MnO₂ (1710 mg, 19.7 mmol) was added to a solution of [3-bromo-5-(trifluoromethyl)-1H-pyrazolo[3,4-c]pyridin-7-yl]methanol (Intermediate 5) (388 mg, 1.31 mmol) in 1,4-dioxane (15.0 mL, c=0.12 M). The mixture was heated at 70° C. for 16 hours and the mixture was filtered through Celite and washed with DCM. The filtrate was concentrated, and the crude was purified by flash column chromatography (ISCO, solvent 0-100% EtOAc/heptane) to give 3-bromo-5-(trifluoromethyl)-1H-pyrazolo[3,4-c]pyridine-7-carbaldehyde (16a) as a white solid (300 mg, 78%). ¹H NMR (400 MHz, DMSO-d₆) δ 14.89 (s, 1H), 10.18 (s, 1H), 8.51 (s, 1H).

### Step 2: (S)—N-((3-bromo-5-(trifluoromethyl)-1H-pyrazolo[3,4-c]pyridin-7-yl)methyl)-3,3-difluorocyclopentan-1-amine (Intermediate 16)

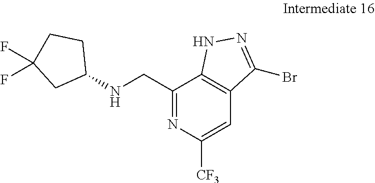 [SMILES: FC1(F)CC[C@H](NCc2nc(C(F)(F)F)cc3c(Br)n[nH]c23)C1]

To a suspension of (S)-3,3-difluorocyclopentanamine·HCl (41.5 mg, 0.263 mmol) in THF (2.0 mL, c=0.1 M) was added Et₃N (127 mg, 1.25 mmol, 0.175 mL), 3-bromo-5-(trifluoromethyl)-1H-pyrazolo[3,4-c]pyridine-7-carbaldehyde (16a) (100 mg, 0.340 mmol) and Ti(OEt)₄ (465 mg, 2.04 mmol, 0.428 mL). The resulting reaction mixture was stirred at 50° C. overnight. The mixture was cooled to RT, added NaBH₃CN (78.7 mg, 1.25 mmol). The reaction was stirred at RT for 1 hr. The reaction was quenched with saturated aqueous Na₂CO₃ (10 mL), filtered and the aqueous layer was extracted with DCM (5 mL×3). The combined organic layers were dried over Na₂SO₄, filtered and concentrated. The crude was purified with by flash column chromatography (ISCO, solvent EtOAc) to give (S)—N-((3-bromo-5-(trifluoromethyl)-1H-pyrazolo[3,4-c]pyridin-7-yl)methyl)-3,3-difluorocyclopentan-1-amine (Intermediate 16) as a dark brown color solid (76.0 mg, 56%). ¹H NMR (400 MHz, CDCl₃) δ 7.90 (s, 1H), 4.44 (d, J=1.8 Hz, 2H), 3.44 (t, J=6.4 Hz, 1H), 2.56-2.19 (m, 4H), 2.18-2.08 (m, 2H), 1.83-1.67 (m, 1H); m/z (APCI+) for (C₁₃H₁₂BrF₅N₄) 400.9 (M+H).

### Preparation of Intermediate 17: 3-{cyclohexyl[3-(4,4,5,5-tetramethyl-1,3,2-dioxaborolan-2-yl)phenyl]methyl}-4-methyl-4H-1,2,4-triazole

### Step 1: Methyl (3-bromophenyl)(cyclohexyl)acetate (17a)

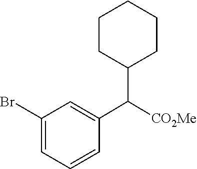 [SMILES: COC(=O)C(c1cccc(Br)c1)C1CCCCC1]

To a solution of methyl (3-bromophenyl)acetate (53.0 g, 231 mmol, 1.0 equiv) in DMF (450 mL) was added potassium tert-butoxide (31.2 g, 278 mmol, 1.2 equiv) portionwise at 0° C. The reaction was stirred at 0° C. for 30 min, then bromocyclohexane (49.04 g, 301 mmol, 1.3 equiv) in DMF (50 mL) was added dropwise at 0° C. The reaction was stirred at 25° C. for 16 h. The reaction was diluted with H₂O (500 mL) and the layers were separated. The aqueous layer was extracted with EtOAc (3×1000 mL). The combined organic layers were washed with brine, dried over Na₂SO₄, filtered, and the filtrate was concentrated under reduced pressure. The crude residue was purified via flash column chromatography (SiO₂, PE:EtOAc=1:0 to 5:1) to give methyl (3-bromophenyl)(cyclohexyl)acetate (17a) (72 g, 90%) as a yellow oil. ¹H NMR (400 MHz, CDCl₃) δ 7.50 (t, J=1.6 Hz, 1H), 7.40 (br d, J=7.9 Hz, 1H), 7.28 (d, J=6.9 Hz, 1H), 7.21-7.15 (m, 1H), 3.67 (s, 3H), 3.20 (d, J=10.5 Hz, 1H), 1.99 (tq, J=3.2, 11.0 Hz, 1H), 1.84-1.72 (m, 2H), 1.69-1.58 (m, 2H), 1.33 (td, J=3.5, 12.6 Hz, 2H), 1.20-1.12 (m, 2H), 1.11-1.00 (m, 1H), 0.82-0.69 (m, 1H).

### Step 2: (3-bromophenyl)(cyclohexyl)acetic acid (17b)

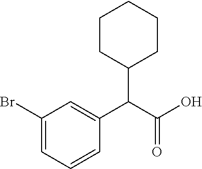 [SMILES: O=C(O)C(c1cccc(Br)c1)C1CCCCC1]

To a solution of methyl (3-bromophenyl)(cyclohexyl)acetate (17a) (71.6 g, 230.07 mmol) in EtOH (230 mL) was added the solution of NaOH (46.01 g, 1.15 mmol) in H₂O (575 mL), and the solution was stirred at 25° C. for 16 h. The solution was concentrated to remove the EtOH and the residue was adjusted to pH ˜3-4 by HCl (4 M). The aqueous layer was extracted with EtOAc (3×600 mL) and the combined organic layers was dried over Na₂SO₄, filtrated and concentrated under reduced pressure to afford (3-bromophenyl)(cyclohexyl)acetic acid (17b) (50 g, 59%) as a yellow oil. The crude was used to next step directly and without further purification.

### Step 3: 2-[(3-bromophenyl)(cyclohexyl)acetyl]-N-methylhydrazine-1-carbothioamide (17c)

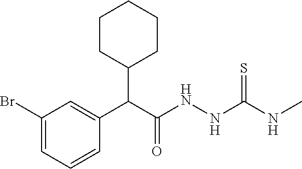 [SMILES: CNC(=S)NNC(=O)C(c1cccc(Br)c1)C1CCCCC1]

To a solution of (3-bromophenyl)(cyclohexyl)acetic acid (17b) (10 g, 33.7 mmol, 1.0 equiv) in DMF (100 mL) at 0° C. was added N-methylhydrazinecarbothioamide (5.31 g, 50.47 mmol, 1.5 equiv), HATU (16.63 g, 43.74 mmol, 1.3 equiv), and i-Pr₂NEt (13.05 g, 100.95 mmol, 3.0 equiv) and the reaction was stirred at 25° C. for 16 h. Then, the reaction was poured into H₂O (200 mL), and the precipitate was filtered to obtain 2-[(3-bromophenyl)(cyclohexyl)acetyl]-N-methylhydrazine-1-carbothioamide (17c) (12 g, 74%) as a white solid. The crude was used to next step directly and without further purification.

### Step 4: 5-[(3-bromophenyl)(cyclohexyl)methyl]-4-methyl-4H-1,2,4-triazole-3-thiol (17d)

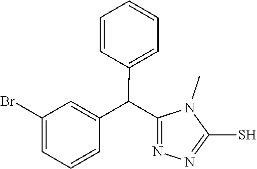 [SMILES: Cn1c(S)nnc1C(c1ccccc1)c1cccc(Br)c1]

To a solution of 2-[(3-bromophenyl)(cyclohexyl)acetyl]-N-methylhydrazine-1-carbothioamide (17c) (12.0 g, 31.2 mmol) in THF (120 mL) was added NaOH (7.49 g, 187 mmol) in H₂O (23 mL) and the solution was stirred at 80° C. for 60 h. The solution was concentrated to remove the THF, diluted with H₂O (80 mL), and the pH was adjusted to ˜2-3 by HCl (2 M). The solid was precipitated out, filtered, and then the solid was dissolved in MeCN, and the resulting solution was concentrated to give crude 5-[(3-bromophenyl)(cyclohexyl)methyl]-4-methyl-4H-1,2,4-triazole-3-thiol (17d) (7.8 g, 55%) as white solid. The crude was used to next step directly and without further purification.

### Step 5: 3-[(3-bromophenyl)(cyclohexyl)methyl]-4-methyl-4H-1,2,4-triazole (17e)

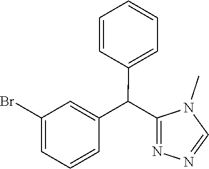 [SMILES: Cn1cnnc1C(c1ccccc1)c1cccc(Br)c1]

To a solution of acetic acid (8.7 mL) in DCM (7 mL) was added hydrogen peroxide (30%, 6.52 mL) at 0-5° C. Then the reaction was warmed to 25° C. and stirred for 5 min before cooling back to 0° C. 5-[(3-bromophenyl)(cyclohexyl)methyl]-4-methyl-4H-1,2,4-triazole-3-thiol (17d) (7.8 g, 21.29 mmol) in DCM (39 mL) was added at 0-5° C. and then warmed to 25° C. After 2 h, the reaction was quenched with saturated sodium sulfite and stirred for 16 h. The layers were separated, and the organic layer was concentrated to remove most of the solvent. H₂O (70 mL) was added, and the reaction was basified to pH ˜8 using saturated aqueous sodium carbonate. The mixture was extracted with EtOAc (3×80 mL). The combined organic layers were dried over Na₂SO₄, filtered, and concentrated under reduced pressure. The crude residue was purified by flash column chromatography (SiO₂, EtOAc/MeOH 2-5%) to give 3-[(3-bromophenyl)(cyclohexyl)methyl]-4-methyl-4H-1,2,4-triazole (17e) (3.67 g, 52%) as a white solid. LCMS (ESI+) 334.2 (M+H)⁺. ¹H NMR (400 MHz, DMSO-d₆) δ 8.33 (s, 1H), 7.58 (s, 1H), 7.41 (dd, J=13.6, 7.9 (m, 2H), 7.31-7.24 (m, 2H), 4.22 (d, 1H), 3.41 (s, 3H), 3.14-3.04 (m, 1H), 2.09-1.99 (m, 1H), 1.83-1.64 (m, 5H).

The racemic compound could be purified into its enantiomers via preparative SFC (Column: Daicel ChiralPak IG, 250×50 mm, 10 um; Flow rate: 230 mL/min; 30% MeOH (with 0.1% NH₄₀H) in CO₂). Peak 1: 3-[(S)-(3-bromophenyl)(cyclohexyl)methyl]-4-methyl-4H-1,2,4-triazole (17′e) was isolated as a white solid (>99% ee). [α]_{D}²² +58.9° (c 0.5, MeOH). Peak 2: 3-[(R)-(3-bromophenyl)(cyclohexyl)methyl]-4-methyl-4H-1,2,4-triazole (17′f) was isolated as a white solid (99% ee). [α]_{D}²² −62.6° (c 0.5, MeOH).

### Step 6: 3-{cyclohexyl[3-(4,4,5,5-tetramethyl-1,3,2-dioxaborolan-2-yl)phenyl]methyl}-4-methyl-4H-1,2,4-triazole (Intermediate 17)

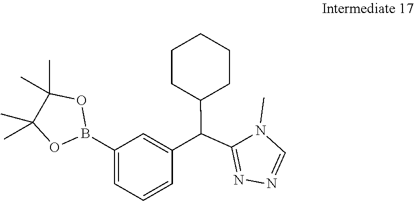 [SMILES: Cn1cnnc1C(c1cccc(B2OC(C)(C)C(C)(C)O2)c1)C1CCCCC1]

A solution of 3-[(3-bromophenyl)(cyclohexyl)methyl]-4-methyl-4H-1,2,4-triazole (17e) (1000 mg, 2.99 mmol, 1.0 equiv), bis(pinacolato)diboron (760 mg, 2.99 mmol, 1.0 equiv), potassium acetate (881 mg, 8.98 mmol, 3.0 equiv), and [1,1′-bis(diphenylphosphino)ferrocene]dichloropalladium(II) (219 mg, 0.299 mmol, 0.10 equiv) in dioxane (15 mL) was degassed with N₂ and stirred at 90° C. for 16 h. The reaction mixture was cooled and concentrated under reduced pressure. The crude residue was purified by flash column chromatography (ISCO, 40 g SiO₂, DCM/MeOH 0-10%) to give 3-{cyclohexyl[3-(4,4,5,5-tetramethyl-1,3,2-dioxaborolan-2-yl)phenyl]methyl}-4-methyl-4H-1,2,4-triazole (Intermediate 17) (1030 mg, 90%) as a yellow gum. LCMS (ESI+) 382.1 (M+H)⁺. ¹H NMR (400 MHz, DMSO-d₆) δ 8.30 (s, 1H), 7.64 (s, 1H), 7.51 (dd, J=12.8, 7.6 Hz, 2H) 7.36-7.28 (m, 1H), 3.92 (br d, J=10.0 Hz, 1H), 3.46 (s, 3H), 2.25-2.10 (m, 1H), 1.62 (br d, J=9.8 Hz, 4H), 1.29 (s, 12H), 1.26-1.18 (m, 2H), 1.15-1.09 (m, 2H), 1.04-0.80 (m, 2H).

### Preparation of Intermediate 17′: 3-{(R)-cyclohexyl[3-(4,4,5,5-tetramethyl-1,3,2-dioxaborolan-2-yl)phenyl]methyl}-4-methyl-4H-1,2,4-triazole

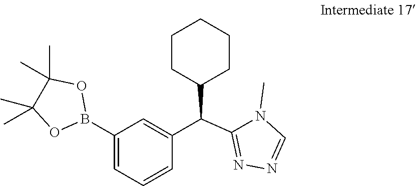 [SMILES: Cn1cnnc1[C@@H](c1cccc(B2OC(C)(C)C(C)(C)O2)c1)C1CCCCC1]

The chiral boronate Intermediate 17′ was made in a similar fashion as detailed above for Intermediate 17 starting from 3-[(R)-(3-bromophenyl)(cyclohexyl)methyl]-4-methyl-4H-1,2,4-triazole (17′e).

### Preparation of Intermediate 18: 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-one

### Step 1: 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-ol (18a)

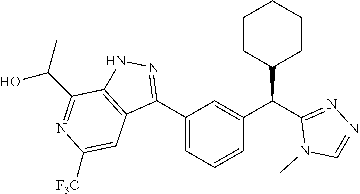 [SMILES: CC(O)c1nc(C(F)(F)F)cc2c(-c3cccc([C@H](c4nncn4C)C4CCCCC4)c3)n[nH]c12]

To a solution of 1-(3-iodo-5-(trifluoromethyl)-1H-pyrazolo[3,4-c]pyridin-7-yl)ethan-1-01 (Intermediate 6′) (231 mg, 0.648 mmol) and 3-[(R)-(3-bromophenyl)(cyclohexyl)methyl]-4-methyl-4H-1,2,4-triazole (Intermediate 17′) (247.0 mg, 0.648 mmol) in iso-propanol (6.48 mL, 0.1 M) was added aqueous potassium phosphate (344 mg, 1.62 mmol, 1.62 mL, 1.0 M) and cataCXium® A Pd G3 (47.2 mg, 0.065 mmol). The mixture was degassed three times and heated at 110° C. overnight.

The mixture was cooled to room temperature, diluted with brine (10 mL) and extracted with DCM (2×10 mL). The organic layers were dried over Na₂SO₄, filtered, and concentrated under reduced pressure. The residue was purified by flash column chromatography (ISCO, SiO₂, 0-10% MeOH/1:1 DCM/EtOAc) to afford 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-ol (18a) as an off-white solid (192 mg, 61%). LCMS (APCI+) 485.2 (M+H)⁺. ¹H NMR (400 MHz, CDCl₃) δ 13.15-12.59 (m, 1H), 8.24-8.19 (m, 1H), 8.13 (d, J=3.8 Hz, 1H), 8.00 (d, J=6.5 Hz, 1H), 7.84-7.77 (m, 1H), 7.49 (dt, J=1.7, 7.7 Hz, 1H), 7.34 (br d, J=7.1 Hz, 1H), 5.61 (qd, J=6.7, 13.8 Hz, 1H), 3.70 (dd, J=2.2, 10.4 Hz, 1H), 3.60 (d, J=1.9 Hz, 3H), 2.73-2.60 (m, 1H), 1.86-1.77 (m, 4H), 1.72-1.57 (m, 6H), 1.23-1.09 (m, 2H), 1.03-0.87 (m, 2H).

### Step 2: 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-one (Intermediate 18)

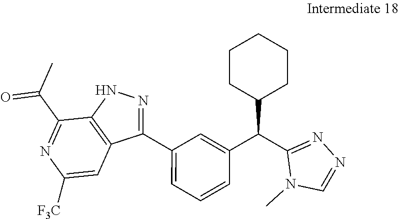 [SMILES: CC(=O)c1nc(C(F)(F)F)cc2c(-c3cccc([C@H](c4nncn4C)C4CCCCC4)c3)n[nH]c12]

To a solution of 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-ol (18a) (190 mg, 0.392 mmol) in CHCl₃ (4.9 mL, c=0.08 M) was added MnO₂ (1.02 g, 11.8 mmol). The resulting mixture was heated at 70° C. for 24 h. The mixture was cooled to RT, filtered through Celite, and washed with CH₂Cl₂. The filtrate was concentrated and purified by flash column chromatography (ISCO, 12 g SiO₂, 0-10% DCM/MeOH to give 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-one (Intermediate 18) as a yellow solid (179 mg, 95%). ¹H NMR (400 MHz, CDCl₃) δ 11.85 (s, 1H), 8.48 (s, 1H), 8.04 (s, 1H), 7.96 (s, 1H), 7.86-7.77 (m, 1H), 7.59-7.45 (m, 2H), 3.75 (d, J=9.8 Hz, 1H), 3.55 (s, 3H), 2.93 (s, 3H), 2.71-2.45 (m, 1H), 1.84-1.66 (m, 4H), 1.49-1.37 (m, 1H), 1.32-1.15 (m, 3H), 1.10-0.93 (m, 2H).

### Preparation of Intermediate 19: 1-(3-bromo-5-cyclopropyl-1H-pyrazolo[3,4-c]pyridin-7-yl)ethan-1-ol

### Step 1: 1-(5-bromo-1H-pyrazolo[3,4-c]pyridin-7-yl)ethan-1-ol (19a)

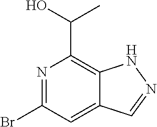

To a mixture of 5-bromo-1H-pyrazolo[3,4-c]pyridine (10 g, 50.5 mmol, 1 equiv), and sulfuric acid (495 mg, 5.05 mmol, 0.1 equiv) in water (50 mL) and ethanol (200 mL) was added ammonium persulfate (8 g, 35 mmol) in water (10 mL). The mixture was stirred at 70° C. for 1 h. An additional portion of ammonium persulfate (8 g, 35 mmol) in water (10 mL) was added every 15 minutes. Then the reaction was cooled to room temperature and volatiles were removed under reduced pressure. EtOAc (100 mL) and water (100 mL) were added, and the layers were separated. The aqueous layer was extracted with EtOAc (2×100 mL). The combined organic layers were sequentially washed with saturated aqueous NaHCO₃ (200 mL) and brine (100 mL), dried over Na₂SO₄, filtered, and concentrated under reduced pressure. The crude residue was purified via flash column chromatography (ISCO, 120 g SiO₂, 0-50% pet ether/EtOAc) to afford 1-(5-bromo-1H-pyrazolo[3,4-c]pyridin-7-yl)ethan-1-ol (19a) (7.5 g, 61%) as a yellow solid. ¹H NMR (400 MHz, DMSO-d₆) δ 13.59 (br s, 1H), 8.17 (s, 1H), 7.90 (s, 1H), 5.83 (d, J=4.5 Hz, 1H), 5.19-4.94 (m, 1H), 1.50 (d, J=6.6 Hz, 3H).

### Step 2: 1-(5-cyclopropyl-1H-pyrazolo[3,4-c]pyridin-7-yl)ethan-1-ol (19b)

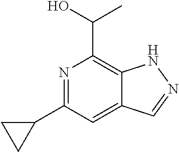

To a solution of 1-(5-bromo-1H-pyrazolo[3,4-c]pyridin-7-yl)ethan-1-ol (19a) (7.5 g, 31.0 mmol, 1.0 equiv) in DMF (120 mL) was added NaH (1.49 g, 37.2 mmol, 1.2 equiv) at 0° C. The reaction was stirred for 20 min at 0° C. Then, 2-(trimethylsilyl)ethoxymethyl chloride (7.75 g, 46.5 mmol, 1.5 equiv) was added at 0° C. and the reaction was warmed to RT and stirred for 1 h. The reaction was quenched with water (20 mL). The reaction mixture was extracted with EtOAc (2×80 mL). The combined organic layers were washed with brine (4×100 mL), dried over Na₂SO₄, filtered, and concentrated under reduced pressure. The crude residue was purified via flash column chromatography (ISCO, 80 g SiO₂, 0-30% pet ether/EtOAc) to afford a mixture of N-isomers (4.46 g and 5.73 g, 39% and 50%) each as a yellow gum. The minor product was carried forward to the next step. LCMS (ESI+) 374.2 (M+H)⁺.

A solution of the minor product from above (6.36 g, 17.0 mmol, 1.0 equiv, combination of multiple batches), XPhos Pd G2 (668 mg, 0.85 mmol, 0.05 equiv), cyclopropylboronic acid (7.30 g, 85.0 mmol, 5.0 equiv), XPhos (405 mg, 0.850 mmol, 0.05 equiv), and K₃PO₄ (10.8 g, 51.0 mmol, 3.0 equiv), in toluene (80 mL) and water (16 mL) was degassed with N₂ and stirred at 100° C. for 16 h. Then, the mixture was concentrated and the residue was dissolved in water (120 mL). The aqueous layer was extracted with EtOAc (2×100 mL). The combined organic layers were washed with brine, dried over Na₂SO₄, filtered, and concentrated under reduced pressure to afford a yellow oil (5.67 g). LCMS (ESI+) 334.2 (M+H)⁺.

To a solution of the yellow oil from above (5.67 g, 17.0 mmol, 1.0 equiv) in DCM (100 mL) was added TFA (20 mL). The mixture was stirred at 25° C. for 3 h. An additional 20 mL of TFA was added and the mixture was stirred at 25° C. for 3 h. Then, the mixture was concentrated and the residue was dissolved in MeOH (100 mL) and NH₃·H₂O (30 mL) and stirred for 16 h at RT. The mixture was concentrated under reduced pressure and purified via flash column chromatography (ISCO, 80 g SiO₂, 0-10% DCM/MeOH) to afford 1-(5-cyclopropyl-1H-pyrazolo[3,4-c]pyridin-7-yl)ethan-1-ol (19b) (2.8 g, 32% from 19a) as a yellow solid. LCMS (ESI+) 204.1 (M+H)⁺. ¹H NMR (400 MHz, CDCl₃) δ 11.14-10.67 (m, 1H), 8.04 (s, 1H), 7.36 (s, 1H), 5.34 (d, J=6.6 Hz, 1H), 2.18 (br t, J=6.9 Hz, 1H), 1.68 (d, J=6.6 Hz, 3H), 1.02-0.97 (m, 4H).

### Step 3: 1-(3-bromo-5-cyclopropyl-1H-pyrazolo[3,4-c]pyridin-7-yl)ethan-1-ol (Intermediate 19)

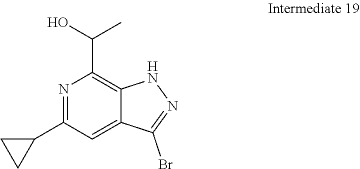 [SMILES: CC(O)c1nc(C2CC2)cc2c(Br)n[nH]c12]

To a solution of 1-(5-cyclopropyl-1H-pyrazolo[3,4-c]pyridin-7-yl)ethan-1-ol (19b) (2.8 g, 13.8 mmol, 1.0 equiv) in DMF (100 mL) was added NBS (2.94 g, 16.5 mmol, 1.2 equiv) and the reaction was stirred at RT. After 16 h, the reaction was quenched with water (100 mL). The aqueous layer was extracted with EtOAc (2×150 mL). The combined organic layers were washed with brine (4×150 mL), dried over Na₂SO₄, filtered, and concentrated under reduced pressure. The crude residue was purified via flash column chromatography (Isco, SiO₂, DCM/MeOH 0-10%) to afford 1-(3-bromo-5-cyclopropyl-1H-pyrazolo[3,4-c]pyridin-7-yl)ethan-1-ol (Intermediate 19) as a yellow solid (3.29 g, 85%). LCMS (ESI+) 281.9, 284.0 (M+H)⁺. ¹H NMR (400 MHz, DMSO-d₆) δ 13.58-13.42 (m, 1H), 7.28 (s, 1H), 5.78-5.64 (m, 1H), 5.08-4.91 (m, 1H), 2.25-2.16 (m, 1H), 1.46 (d, J=6.6 Hz, 3H), 0.96-0.85 (m, 4H).

### Preparation of Intermediate 20: 1-(3-{3-[(R)-cyclobutyl(4-methyl-4H-1,2,4-triazol-3-yl)methyl]phenyl}-5-cyclopropyl-1H-pyrazolo[3,4-c]pyridin-7-yl)ethan-1-one

### Step 1: 1-(3-{3-[(R)-cyclobutyl(4-methyl-4H-1,2,4-triazol-3-yl)methyl]phenyl}-5-cyclopropyl-1H-pyrazolo[3,4-c]pyridin-7-yl)ethan-1-ol (20a)

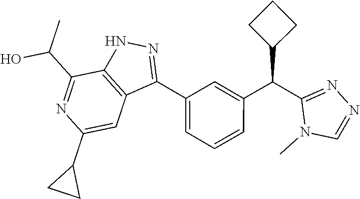 [SMILES: CC(O)c1nc(C2CC2)cc2c(-c3cccc([C@H](c4nncn4C)C4CCC4)c3)n[nH]c12]

A solution of 1-(3-bromo-5-cyclopropyl-1H-pyrazolo[3,4-c]pyridin-7-yl)ethan-1-ol (Intermediate 19) (500 mg, 1.77 mmol), 3-{(R)-cyclobutyl[3-(4,4,5,5-tetramethyl-1,3,2-dioxaborolan-2-yl)phenyl]methyl}-4-methyl-4H-1,2,4-triazole (Intermediate 1′) (689 mg, 1.95 mmol), CsF (592 mg, 3.90 mmol), and [1,1′-bis(diphenylphosphino)ferrocene]dichloropalladium(II) (130 mg, 0.177 mmol) in dioxane (10 mL) and water (2 mL) was degassed with N₂ and heated to 95° C. for 16 h. The mixture was then cooled to room temperature and concentrated under reduced pressure. The residue was purified by flash column chromatography (ISCO, 20 g SiO₂, 0-10% DCM/MeOH) to afford 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-ol (20a) as a brown solid (507 mg, 67%). LCMS (ESI+) 429.3 (M+H)⁺. ¹H NMR (400 MHz, DMSO-d₆) δ 13.39-13.25 (m, 1H), 8.39-8.33 (m, 1H), 7.89-7.83 (m, 2H), 7.64-7.61 (m, 1H), 7.50-7.44 (m, 1H), 7.31-7.27 (m, 1H), 5.09-5.01 (m, 1H), 4.39-4.30 (m, 1H), 3.45 (s, 3H), 3.26-3.16 (m, 1H), 2.20 (s, 1H), 2.16-2.06 (m, 1H), 1.90-1.72 (m, 5H), 1.49 (d, J=6.6 Hz, 3H), 0.97-0.89 (m, 4H).

### Step 2: 1-(3-{3-[(R)-cyclobutyl(4-methyl-4H-1,2,4-triazol-3-yl)methyl]phenyl}-5-cyclopropyl-1H-pyrazolo[3,4-c]pyridin-7-yl)ethan-1-one (Intermediate 20)

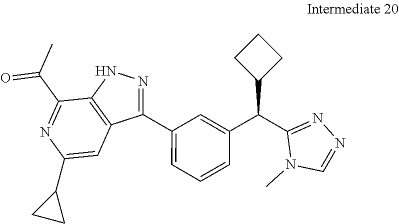 [SMILES: CC(=O)c1nc(C2CC2)cc2c(-c3cccc([C@H](c4nncn4C)C4CCC4)c3)n[nH]c12]

To a solution of 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-ol (20a) (200 mg, 0.467 mmol) in dioxane (5 mL) was added MnO₂ (406 mg, 4.67 mmol). The resulting mixture was heated at 80° C. for 16 h. The mixture was cooled to RT, dissolved in EtOAc, and filtered through Celite. The filtrate was concentrated to give 1-(3-{3-[(R)-cyclobutyl(4-methyl-4H-1,2,4-triazol-3-yl)methyl]phenyl}-5-cyclopropyl-1H-pyrazolo[3,4-c]pyridin-7-yl)ethan-1-one (Intermediate 20) as a brown solid (200 mg, >99%). The crude material was carried to the next step without further purification. LCMS (ESI+) 427.2 (M+H)⁺. ¹H NMR (400 MHz, DMSO-d₆) δ 13.84 (s, 1H), 8.40-8.32 (m, 1H), 8.16 (s, 1H), 7.93 (br s, 2H), 7.54-7.45 (m, 1H), 7.33 (br d, J=8.1 Hz, 1H), 4.43-4.29 (m, 1H), 3.48-3.43 (m, 3H), 3.24 (br s, 1H), 2.71 (s, 3H), 2.40-2.29 (m, 1H), 2.17-2.05 (m, 1H), 1.81 (br s, 5H), 1.06 (br s, 4H).

### Preparation of Intermediate 21: 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-one

### Step 1: 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol (21a)

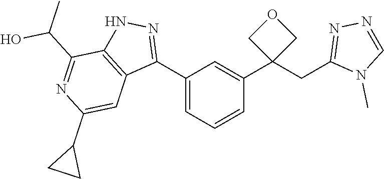 [SMILES: CC(O)c1nc(C2CC2)cc2c(-c3cccc(C4(Cc5nncn5C)COC4)c3)n[nH]c12]

A solution of 1-(3-bromo-5-cyclopropyl-1H-pyrazolo[3,4-c]pyridin-7-yl)ethan-1-ol (Intermediate 19) (500 mg, 1.77 mmol), 4-methyl-3-({3-[3-(4,4,5,5-tetramethyl-1,3,2-dioxaborolan-2-yl)phenyl]oxetan-3-yl}methyl)-4H-1,2,4-triazole (Intermediate 2) (693 mg, 1.95 mmol), CsF (592 mg, 3.90 mmol), and [1,1′-bis(diphenylphosphino)ferrocene]dichloropalladium(II) (130 mg, 0.177 mmol) in dioxane (15 mL) and water (3 mL) was degassed with N₂ and heated to 95° C. for 16 h. The mixture was then cooled to room temperature and concentrated under reduced pressure. The residue was purified by flash column chromatography (ISCO, 12 g SiO₂, 0-10% DCM/MeOH) to afford 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol (21a) as a brown solid (540 mg, 71%). LCMS (ESI+) 431.2 (M+H)⁺. ¹H NMR (400 MHz, DMSO-d₆) δ 13.31 (s, 1H), 8.22 (s, 1H), 7.87-7.80 (m, 1H), 7.54 (s, 1H), 7.48 (br d, J=13.6 Hz, 2H), 7.07-7.03 (m, 1H), 5.70-5.64 (m, 1H), 5.07-5.04 (m, 1H), 5.02 (br d, J=5.9 Hz, 2H), 4.96 (d, J=6.0 Hz, 2H), 3.58 (s, 2H), 2.94 (s, 3H), 2.32-2.27 (m, 1H), 1.48 (d, J=6.6 Hz, 3H), 0.98-0.86 (m, 4H).

### Step 2: 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-one (Intermediate 21)

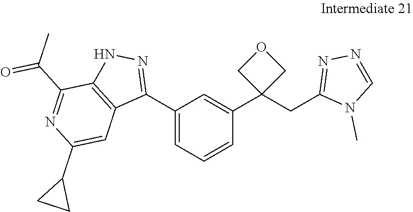 [SMILES: CC(=O)c1nc(C2CC2)cc2c(-c3cccc(C4(Cc5nncn5C)COC4)c3)n[nH]c12]

To a solution of 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol (21a) (540 mg, 1.25 mmol) in dioxane (10 mL) was added MnO₂ (1.09 g, 12.5 mmol). The resulting mixture was heated at 80° C. for 16 h. The mixture was cooled to RT, dissolved in EtOAc, and filtered through Celite. The filtrate was concentrated to give 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-one (Intermediate 21) as a brown solid (500 mg, 93%). The crude material was carried to the next step without further purification. LCMS (ESI+) 429.1 (M+H)⁺. ¹H NMR (400 MHz, DMSO-d₆) δ 13.83 (s, 1H), 8.24 (s, 1H), 8.10-8.05 (m, 1H), 7.91-7.84 (m, 1H), 7.60-7.56 (m, 1H), 7.52-7.45 (m, 1H), 7.14-7.06 (m, 1H), 5.04-5.00 (m, 2H), 4.99-4.95 (m, 2H), 3.62-3.59 (m, 2H), 3.57 (s, 1H), 3.01-2.96 (m, 3H), 2.71 (s, 3H), 1.12-0.99 (m, 4H).

### Preparation of Intermediate 22: 1-(3-{3-[(R)-cyclobutyl(4-methyl-4H-1,2,4-triazol-3-yl)methyl]phenyl}-1H-pyrazolo[3,4-c]pyridin-7-yl)ethan-1-one

### Step 1: 1-(1H-pyrazolo[3,4-c]pyridin-7-yl)ethan-1-one (22a)

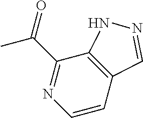

To a suspension of 7-chloro-1H-pyrazolo[3,4-c]pyridine (3.99 g, 26.0 mmol, 1 equiv) and bis(triphenylphosphine)palladium(II) dichloride (3.1 g, 4.4 mmol, 17 mol %) in 1,4-dioxane (130 mL) under nitrogen atmosphere was added tributyl(1-ethoxyvinyl)tin (15.0 g, 41.6 mmol, 14.0 mL, 1.6 equiv) and the mixture was heated at 100° C. for 17 h. The mixture was cooled to room temperature, diluted with water (50 mL) and EtOAc (50 mL), and potassium fluoride (4 g) was added. The mixture was stirred at room temperature for 1 h, after which the layers were separated, the aqueous layer was extracted with EtOAc (50 mL), and the combined organic layer was washed with brine (2×40 mL) and dried over sodium sulfate. The sodium sulfate was filtered off, and 6 N HCl (30 mL) was added, and the mixture was stirred at room temperature for 1.5 h. Additional 12 N HCl (50 mL) was added, and the mixture was stirred at room temperature for 1 h. The aqueous layer was neutralized with 5 N aqueous NaOH until the pH=7, and the aqueous layer was extracted with EtOAc (3×50 mL). The combined organic layers were dried over sodium sulfate, filtered and concentrated. The crude residue was purified via flash chromatography (ISCO 80 g SiO₂, ethyl acetate/heptanes 0-100%) to give 1-(1H-pyrazolo[3,4-c]pyridin-7-yl)ethan-1-one (22a) as a yellow solid (2.00 g, 48%). LCMS (ESI+) 162.1 (M+H)⁺. ¹H NMR (400 MHz, DMSO-d₆) δ 14.01-13.69 (m, 1H), 8.49-8.25 (m, 2H), 8.17-8.02 (m, 1H), 2.75 (s, 3H).

### Step 2: 1-(1H-pyrazolo[3,4-c]pyridin-7-yl)ethan-1-ol (22b)

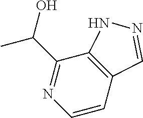

To a suspension of 1-(1H-pyrazolo[3,4-c]pyridin-7-yl)ethan-1-one (22a) (991 mg, 6.15 mmol, 1 equiv), (3R)-oxolan-3-amine (540 mg, 6.20 mmol, 0.54 mL, 1 equiv) in EtOH (61.5 mL) under nitrogen atmosphere was added MgSO₄ (1476 mg, 12.26 mmol, 2 equiv) and the mixture was stirred at room temperature for 17 h. Acetic acid (36.8 mg, 0.612 mmol, 35.0 uL, 0.01 equiv) followed by sodium borohydride (797 mg, 21.1 mmol, 3.4 equiv) and (3R)-oxolan-3-amine (540 mg, 6.20 mmol, 0.54 mL, 1 equiv) were added. The mixture was concentrated and then diluted with EtOH (50 mL) and 1 N HCl (30 mL), and the reaction was stirred at room temperature for 25 h. The mixture was neutralized with 1 N NaOH until the pH=9, and the product was extracted with EtOAc (3×100 mL). The combined organic layers were washed with brine (100 mL) and concentrated. The crude residue was purified by flash chromatography (ISCO, 80 g SiO₂, ethyl acetate/heptanes 0-100%) to give 1-(1H-pyrazolo[3,4-c]pyridin-7-yl)ethan-1-ol (22b) as a white solid (680 mg, 69%). LCMS (ESI+) 164.1 (M+H)⁺. ¹H NMR (400 MHz, DMSO-d₆) δ 13.46-13.15 (m, 1H), 8.17 (d, J=1.5 Hz, 1H), 8.09 (d, J=5.6 Hz, 1H), 7.61 (d, J=5.5 Hz, 1H), 5.63 (d, J=4.8 Hz, 1H), 5.22-5.02 (m, 1H), 1.51 (d, J=6.6 Hz, 3H).

### Step 3: 1-(3-iodo-1H-pyrazolo[3,4-c]pyridin-7-yl)ethan-1-ol (22c)

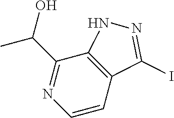

A suspension of 1-(1H-pyrazolo[3,4-c]pyridin-7-yl)ethan-1-ol (22b) (300 mg, 1.84 mmol, 1 equiv) and N-iodosuccinimide (620 mg, 2.76 mmol, 1.5 equiv) in acetonitrile (6.13 mL) under nitrogen atmosphere was stirred at 60° C. for 1.5 h. The mixture was concentrated, diluted with EtOAc (15 mL) washed sequentially with 10% aqueous sodium thiosulfate (5 mL) and brine (5 mL). The organic layer was dried over sodium sulfate and concentrated. The crude residue was purified by flash chromatography to give 1-(3-iodo-1H-pyrazolo[3,4-c]pyridin-7-yl)ethan-1-ol (22c) as a white solid (466 mg, 88%). LCMS (ESI+) 289.9 (M+H)⁺. ¹H NMR (400 MHz, DMSO-d₆) δ 8.23-8.03 (m, 1H), 7.37-7.19 (m, 1H), 5.81-5.69 (m, 1H), 5.18-5.08 (m, 1H), 1.51 (d, J=6.6 Hz, 3H).

### Step 4: 1-(3-{3-[(R)-cyclobutyl(4-methyl-4H-1,2,4-triazol-3-yl)methyl]phenyl}-1H-pyrazolo[3,4-c]pyridin-7-yl)ethan-1-ol (22d)

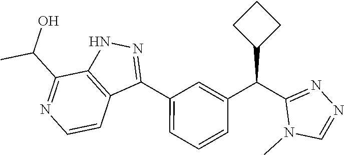 [SMILES: CC(O)c1nccc2c(-c3cccc([C@H](c4nncn4C)C4CCC4)c3)n[nH]c12]

A suspension of 1-(3-iodo-1H-pyrazolo[3,4-c]pyridin-7-yl)ethan-1-ol (22c) (204 mg, 0.71 mmol, 1 equiv), (R)-3-(cyclobutyl(3-(4,4,5,5-tetramethyl-1,3,2-dioxaborolan-2-yl)phenyl)-methyl)-4-methyl-4H-1,2,4-triazole (Intermediate 1′) (275 mg, 0.78 mmol, 1.1 equiv), cataCXium® A Pd G3 (51.4 mg, 10 mol %), and K₃PO₄ (374 mg, 1.76 mmol, 2.5 equiv) in isopropanol (3.6 mL) and water (0.9 mL) under nitrogen atmosphere was stirred at 110° C. for 20 h. The mixture was diluted with brine (2 mL) and extracted with EtOAc (3×7 mL). The combined organic layer was dried over sodium sulfate, filtered, and concentrated. The crude residue was purified by flash chromatography (ISCO, 12 g SiO₂, DCM/MeOH 0-20%) to give 1-(3-{3-[(R)-cyclobutyl(4-methyl-4H-1,2,4-triazol-3-yl)methyl]phenyl}-1H-pyrazolo[3,4-c]pyridin-7-yl)ethan-1-ol (22d) (197 mg, 72%). LCMS (ESI+) 389.2 (M+H)⁺. ¹H NMR (400 MHz, CDCl₃) δ 8.28 (d, J=5.7 Hz, 1H), 8.03 (s, 1H), 7.93-7.81 (m, 2H), 7.73 (dd, J=2.0, 5.6 Hz, 1H), 7.52-7.38 (m, 1H), 5.46 (dq, J=2.3, 6.5 Hz, 1H), 4.08-3.95 (m, 1H), 3.57-3.36 (m, 7H), 2.47-2.28 (m, 1H), 2.03-1.81 (m, 4H), 1.80-1.67 (m, 4H).

### Step 5: 1-(3-{3-[(R)-cyclobutyl(4-methyl-4H-1,2,4-triazol-3-yl)methyl]phenyl}-1H-pyrazolo[3,4-c]pyridin-7-yl)ethan-1-one (Intermediate 22)

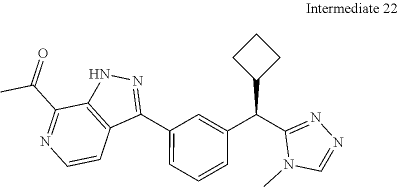 [SMILES: CC(=O)c1nccc2c(-c3cccc([C@H](c4nncn4C)C4CCC4)c3)n[nH]c12]

A suspension of 1-(3-{3-[(R)-cyclobutyl(4-methyl-4H-1,2,4-triazol-3-yl)methyl]phenyl}-1H-pyrazolo[3,4-c]pyridin-7-yl)ethan-1-ol (22d) (195 mg, 0.502 mmol, 1.0 equiv) and manganese dioxide (1310 mg, 15.1 mmol, 30.0 equiv) in DCM (18 mL) was stirred at reflux for 4 days. The mixture was cooled to room temperature, filtered through celite and concentrated to yield 1-(3-{3-[(R)-cyclobutyl(4-methyl-4H-1,2,4-triazol-3-yl)methyl]phenyl}-1H-pyrazolo[3,4-c]pyridin-7-yl)ethan-1-one (Intermediate 22) (102 mg, 53%) which was carried forward without further purification. LCMS (ESI+) 387.1 (M+H)⁺.

### Preparation of Intermediate 23: 4-methyl-3-{1-[3-(4,4,5,5-tetramethyl-1,3,2-dioxaborolan-2-yl)phenyl]propyl}-4H-1,2,4-triazole

### Step 1: 2-(3-bromophenyl)acetohydrazide (23a)

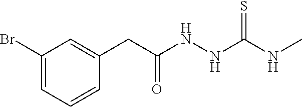 [SMILES: CNC(=S)NNC(=O)Cc1cccc(Br)c1]

To a solution of methyl (3-bromophenyl)acetate (9.49 g, 41.4 mmol, 1.0 equiv) in EtOH (150 mL) was added hydrazine hydrate (22.0 g, 431 mmol). The reaction was stirred at 80° C. for 16 h, then cooled and concentrated under reduced pressure. The crude residue was purified by flash column chromatography (Biotage, 80 g SiO₂, pet ether/EtOAc 0-100%) to give 2-(3-bromophenyl)acetohydrazide (23a) (7.4 g, 78%) as a white solid. ¹H NMR (400 MHz, CDCl₃) δ 7.50-7.40 (m, 2H), 7.26-7.17 (m, 2H), 7.01 (br s, 1H), 3.86-3.61 (m, 2H), 3.52 (s, 2H).

### Step 2: 5-[(3-bromophenyl)methyl]-4-methyl-4H-1,2,4-triazole-3-thiol (23b)

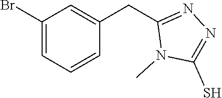

To a solution of 2-(3-bromophenyl)acetohydrazide (23a) (7.4 g, 32.3 mmol, 1.0 equiv) in THF (80 mL) was added isothiocyanatomethane (3.54 g, 48.5 mmol, 1.5 equiv) and the reaction was stirred at 10° C. for 16 h. Then, a solution of NaOH (3.88 g, 96.9 mmol) and water (15 mL) was added. The reaction was stirred at 60° C. for 3 h. The reaction was concentrated under reduced pressure and then diluted with water (30 mL) and acidified to pH 1. The precipitate was filtered and the wet solid was dissolved in MeCN (100 mL) and then concentrated under reduced pressure to give 5-[(3-bromophenyl)methyl]-4-methyl-4H-1,2,4-triazole-3-thiol (23b) (9.1 g, quantitative yield) as an off-white solid. LCMS (ESI+) 283.8 (M+H)⁺. ¹H NMR (400 MHz, DMSO-d₆) δ 13.60 (br s, 1H), 7.60-7.42 (m, 2H), 7.37-7.15 (m, 2H), 4.12 (s, 2H), 3.34 (s, 3H).

### Step 3: 3-[(3-bromophenyl)methyl]-4-methyl-4H-1,2,4-triazole (23c)

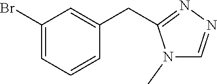

To a solution of acetic acid (12 mL) in DCM (10 mL) was added H₂O₂ (10.9 g, 96.1 mmol, 30%) slowly at 0° C. The reaction was stirred at 15° C. for 5 min, then it was added to another solution of 5-[(3-bromophenyl)methyl]-4-methyl-4H-1,2,4-triazole-3-thiol (23b) (9.10 g, 32.0 mmol) in DCM (50 mL) slowly at 0° C. The reaction was stirred at 15° C. for 2 h. The reaction was quenched with Na₂SO₃ until KI starch paper does not change color. The organic layer was separated and concentrated under reduced pressure. Then, water (40 mL) and aqueous Na₂CO₃ was added to reach pH 8. The aqueous layer was extracted with EtOAc (2×40 mL). The combined organic layers were washed sequentially with brine (50 mL), saturated aqueous NaHCO₃ (3×50 mL), dried over Na₂SO₄, filtered, and concentrated under reduced pressure. The crude residue was slurried with pet ether/EtOAc (10:1, 50 mL) and then purified by flash column chromatography (ISCO, 40 g SiO₂, DCM/MeOH 0-10%) to give 3-[(3-bromophenyl)methyl]-4-methyl-4H-1,2,4-triazole (23c) (800 mg, 10%) as a yellow solid. LCMS (ESI+) 252.1 (M+H)⁺. ¹H NMR (400 MHz, CDCl₃) δ 8.08 (s, 1H), 7.46-7.32 (m, 2H), 7.23-7.08 (m, 2H), 4.19 (s, 2H), 3.48 (s, 3H).

### Step 4: 3-[1-(3-bromophenyl)propyl]-4-methyl-4H-1,2,4-triazole (23d)

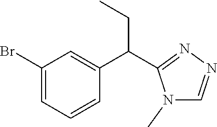 [SMILES: CCC(c1cccc(Br)c1)c1nncn1C]

To a solution of 3-[(3-bromophenyl)methyl]-4-methyl-4H-1,2,4-triazole (23c) (500 mg, 1.98 mmol, 1.0 equiv) and iodoethane (402 mg, 2.58 mmol, 1.3 equiv) in DMF (11 mL) was added t-BuOK (334 mg, 2.97 mmol, 1.5 equiv) portionwise at 0° C. under N₂. The reaction was stirred at 0° C. for 3 h. The reaction mixture was diluted with water (20 mL) and EtOAc (20 mL). The layers were separated and the aqueous layer was extracted with EtOAc (2×20 mL). The combined organic layers were washed with brine (4×30 mL), dried over Na₂SO₄, filtered, and concentrated under reduced pressure. The crude residue was purified by flash column chromatography (ISCO, 20 g SiO₂, DCM/MeOH 0-6%) to give 3-[1-(3-bromophenyl)propyl]-4-methyl-4H-1,2,4-triazole (23d) (420 mg, 76%) as a yellow gum. LCMS (ESI+) 281.9 (M+H)⁺. ¹H NMR (400 MHz, CDCl₃) δ 8.08 (s, 1H), 7.43-7.34 (m, 2H), 7.22-7.12 (m, 2H), 3.83-3.73 (m, 1H), 3.40 (s, 3H), 2.45 (quind, J=7.2, 14.1 Hz, 1H), 2.20-2.08 (m, 1H), 0.98 (t, J=7.4 Hz, 3H).

### Step 5: 4-methyl-3-{1-[3-(4,4,5,5-tetramethyl-1,3,2-dioxaborolan-2-yl)phenyl]propyl}-4H-1,2,4-triazole (Intermediate 23)

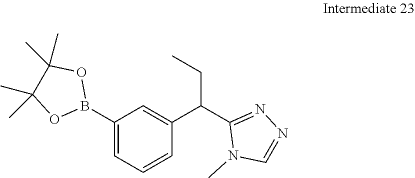 [SMILES: CCC(c1cccc(B2OC(C)(C)C(C)(C)O2)c1)c1nncn1C]

A solution of 3-[1-(3-bromophenyl)propyl]-4-methyl-4H-1,2,4-triazole (23d) (420 mg, 1.50 mmol, 1.0 equiv), bis(pinacolato)diboron (381 mg, 1.50 mmol, 1.0 equiv), potassium acetate (441 mg, 4.50 mmol, 3.0 equiv), and [1,1′-bis(diphenylphosphino)ferrocene]dichloropalladium(II) (0.110 g, 0.150 mmol, 0.10 equiv) in dioxane (15 mL) was degassed with N₂ and stirred at 90° C. for 16 h. The reaction mixture was cooled, filtered, and concentrated under reduced pressure. The crude residue was purified by flash column chromatography (ISCO, 20 g SiO₂, DCM/MeOH 0-6%) to give 4-methyl-3-{1-[3-(4,4,5,5-tetramethyl-1,3,2-dioxaborolan-2-yl)phenyl]propyl}-4H-1,2,4-triazole (Intermediate 23) (560 mg, quant yield) as a yellow gum. LCMS (ESI+) 328.0 (M+H)⁺. ¹H NMR (400 MHz, DMSO-d₆) δ 8.33 (s, 1H), 7.56-7.52 (m, 2H), 7.41-7.37 (m, 1H), 7.36-7.31 (m, 1H), 4.11 (t, J=7.5 Hz, 1H), 3.37 (s, 3H), 2.24-2.14 (m, 1H), 1.96-1.91 (m, 1H), 1.28 (s, 12H), 0.83 (t, J=7.3 Hz, 3H).

### Preparation of Intermediate 24: 5-(difluoromethoxy)-3-iodo-1H-pyrazolo[3,4-c]pyridine

### Step 1: 6-(difluoromethoxy)-4-methylpyridin-3-amine (24a)

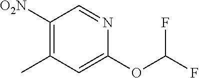

A mixture of 4-methyl-5-nitropyridin-2-ol (1.56 g, 10.1 mmol) and sodium carbonate (1.18 g, 11.1 mmol) in acetonitrile (25.3 mL) was heated to 60° C. The mixture was stirred for 10 minutes before bromodifluoromethyltrimethylsilane (2.47 g, 12.1 mmol, 1.89 mL) was added. The mixture was stirred at 60° C. for 21 h. The reaction mixture was cooled to 10° C. then diluted with EtOAc and water. The EtOAc layer was washed with brine, dried over Na₂SO₄, filtered, and concentrated to an oil. The crude oil was purified by flash column chromatography (ISCO, 40 g SiO₂, EtOAc/heptane 0-50%) to give 2-(difluoromethoxy)-4-methyl-5-nitropyridine (24a) (1.11 g, 54%). LCMS (ESI+) 205.1 (M+H)⁺. ¹H NMR (400 MHz, CDCl₃) δ 8.90 (s, 1H), 7.72-7.31 (m, 1H), 6.87 (s, 1H), 2.69 (s, 3H).

### Step 2: 6-(difluoromethoxy)-4-methylpyridin-3-amine (24b)

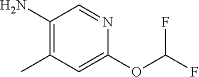

A mixture of 2-(difluoromethoxy)-4-methyl-5-nitropyridine (24a) (1.11 g, 5.438 mmol, 1.00 equiv) and Raney Nickle (1 g) in methanol (20 mL) was hydrogenated under 1 atm (balloon) at 25° C. for 5 h. The reaction mixture was filtered through celite and the filtrate was concentrated to a light tan solid to give 6-(difluoromethoxy)-4-methylpyridin-3-amine (24b) (947 mg, 100%) which was used without further purification. LCMS (ESI+) 175.2 (M+H)⁺. ¹H NMR (400 MHz, CDCl₃) δ 7.59 (s, 1H), 7.50-7.03 (t, 1H), 6.66 (s, 1H), 3.49 (br d, J=7.1 Hz, 2H), 2.20 (d, J=0.6 Hz, 3H).

### Step 3: 1-(5-(difluoromethoxy)-1H-pyrazolo[3,4-c]pyridin-1-yl)ethan-1-one (24c)

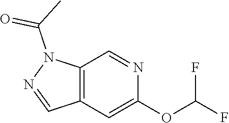 [SMILES: CC(=O)n1ncc2cc(OC(F)F)ncc21]

6-(difluoromethoxy)-4-methylpyridin-3-amine (24b) (947 mg) was dissolved in acetic anhydride (5.14 ml, 54.4 mmol) then stirred at 25° C. for 18 hours. Sodium nitrite (1.88 g, 27.2 mmol) was added and the mixture was heated to 70° C. for 2.5 h. The reaction mixture was cooled to 25° C. then quenched with 10% aqueous sodium thiosulfate. The crude mixture was diluted with EtOAc and water and the EtOAc layer was washed with brine, dried over Na₂S₂O₄, filtered, and concentrated to an orange paste. The crude paste was purified by flash column chromatography (ISCO, 40 g SiO₂, EtOAc/heptane 0-50%) to give 1-(5-(difluoromethoxy)-1H-pyrazolo[3,4-c]pyridin-1-yl)ethan-1-one (24c) (502 mg, 41%). LCMS (ESI+) 228.1 (M+H)⁺. ¹H NMR (400 MHz, CDCl₃) δ 9.41 (s, 1H), 8.17 (s, 1H), 7.52 (t, J=72.0 Hz, 1H), 7.22 (d, J=1.1 Hz, 1H), 2.81 (s, 3H).

### Step 4: 5-(difluoromethoxy)-3-iodo-1H-pyrazolo[3,4-c]pyridine (Intermediate 24)

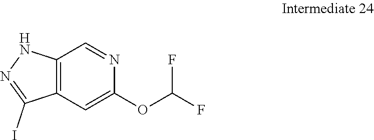 [SMILES: FC(F)Oc1cc2c(I)n[nH]c2cn1]

To a mixture of 1-(5-(difluoromethoxy)-1H-pyrazolo[3,4-c]pyridin-1-yl)ethan-1-one (24c) (363.0 mg, 1.60 mmol) and potassium carbonate (331 mg, 2.40 mmol) in a mixture of DMF (3.0 mL) and methanol (1.0 mL) at 25° C. was added iodine (608 mg, 2.40 mmol). The dark brownish red mixture was stirred at 25° C. for 2 h. The reaction mixture was quenched with 10% aqueous sodium thiosulfate then concentrated under house vacuum. The mixture was then poured into a beaker containing ice and water. A tan solid crashed out which was filtered off using a Buchner funnel. The solid was air dried then diluted with methanol and concentrated to a solid. The solid was then azeotroped in toluene to give 5-(difluoromethoxy)-3-iodo-1H-pyrazolo[3,4-c]pyridine (Intermediate 24) (365 mg, 73%) as a light tan solid. LCMS (ESI+): 311.9 (M+H)⁺. ¹H NMR (400 MHz, CDCl₃) δ 10.72-10.52 (m, 1H), 8.65 (s, 1H), 7.31 (t, J=72.0 Hz, 1H), 7.05 (d, J=0.90 Hz, 1H).

### Preparation of Intermediate 25: 3-iodo-5-(trifluoromethoxy)-1H-pyrazolo[3,4-c]pyridine

### Step 1: 4-methyl-5-nitro-2-(trifluoromethoxy)pyridine (25a)

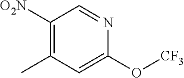

A mixture of 4-methyl-5-nitropyridin-2-ol (0.5 g, 3.24 mmol), 1-(trifluoromethyl)-1,2-benzo[d][1,2]iodaoxol-3(1H)-one (1.71 g, 3.24 mmol) in nitromethane (10.0 mL) was heated to 90° C. in a 50 mL EasyMax reactor for 18 h. The reaction mixture was cooled to 25° C. then diluted with EtOAc (40 mL) and filtered through celite. The filtrate was washed with water (10 mL), brine, dried over Na₂SO₄, filtered then concentrated to an oil.

The crude oil was purified by flash column chromatography (ISCO, 40 g SiO₂, EtOAc/heptane 0-50%) to give 4-methyl-5-nitro-2-(trifluoromethoxy)pyridine (25a) (132 mg, 18%) as a colorless liquid. ¹H NMR (400 MHz, CDCl₃) δ 8.97 (s, 1H), 6.97 (s, 1H), 2.72-2.70 (m, 3H).

### Step 2: 4-methyl-5-nitro-2-(trifluoromethoxy)pyridine (25b)

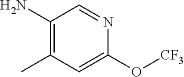

A mixture of 4-methyl-5-nitro-2-(trifluoromethoxy)pyridine (25a) (223 mg, 1 mmol) and iron (280 mg, 5.02 mmol) in acetic acid (2 mL) was heated to 80° C. for 19 h. The reaction mixture was diluted with methanol then filtered through celite. The filtrate was concentrated to an oil to give 4-methyl-5-nitro-2-(trifluoromethoxy)pyridine (25b) (193 mg, quant yield) which was carried forward without further purification. LCMS (ESI+) 193.1 (M+H)⁺. ¹H NMR (400 MHz, DMSO-d₆) δ 7.59 (br s, 1H), 7.02-6.74 (m, 1H), 5.24 (br d, J=1.2 Hz, 2H), 2.11 (s, 3H).

### Step 3: 1-(5-(trifluoromethoxy)-1H-pyrazolo[3,4-c]pyridin-1-yl)ethan-1-one (25c)

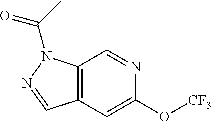 [SMILES: CC(=O)n1ncc2cc(OC(F)(F)F)ncc21]

A mixture 4-methyl-5-nitro-2-(trifluoromethoxy)pyridine (25b) (193 mg, 1.00 mmol) in acetic anhydride (5 mL) was stirred at rt for 10 min then sodium nitrite (347 mg, 5.02 mmol) was added and the mixture was stirred at 90° C. for 18 h. The reaction mixture was concentrated under high vacuum to give an oil. The crude oil was partitioned between EtOAc and water. The EtOAc layer was washed with brine, dried over Na₂SO₄, filtered, and concentrated to an oil. The crude oil was purified by flash column chromatography (ISCO, 4 g Gold SiO₂, EtOAc/heptane 0-100%) to give 1-(5-(trifluoromethoxy)-1H-pyrazolo[3,4-c]pyridin-1-yl)ethan-1-one (25c) (81 mg, 33%) as a yellow solid. LCMS (ESI+) 246.0 (M+H)⁺. ¹H NMR (400 MHz, CDCl₃) δ 9.54 (s, 1H), 8.22 (s, 1H), 7.40 (d, J=1.0 Hz, 1H), 2.82 (s, 3H).

### Step 4: 3-iodo-5-(trifluoromethoxy)-1H-pyrazolo[3,4-c]pyridine (Intermediate 25)

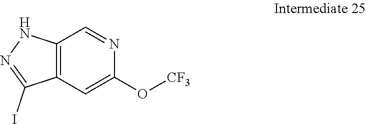 [SMILES: FC(F)(F)Oc1cc2c(I)n[nH]c2cn1]

To a mixture of 1-(5-(trifluoromethoxy)-1H-pyrazolo[3,4-c]pyridin-1-yl)ethan-1-one (25c) (78 mg, 0.32 mmol) in a mixture of DMF (0.7 mL) and methanol (0.3 mL) at rt was added potassium carbonate (66.0 mg, 0.477 mmol) followed by iodine (121 mg, 0.477 mmol). The mixture was stirred at 25° C. for 7 h. The reaction mixture was then quenched with 10% Na₂S₂O₃ (aq) and diluted with EtOAc and water. The EtOAc layer was washed with brine, dried over Na₂SO₄, filtered, and concentrated to an oil. The crude oil was purified by flash column chromatography (ISCO, 4 g Gold SiO₂, EtOAc/heptane 0-100%) to give 3-iodo-5-(trifluoromethoxy)-1H-pyrazolo[3,4-c]pyridine (Intermediate 25) (62 mg, 59%) as a yellow solid. LCMS (ESI+) 330.1 (M+H)⁺. ¹H NMR (400 MHz, CDCl₃) δ 10.94-10.58 (m, 1H), 8.77 (d, J=1.0 Hz, 1H), 7.22 (d, J=0.9 Hz, 1H).

### Preparation of Intermediate 26: 1-[3-{3-[(4-methyl-4H-1,2,4-triazol-3-yl)(oxetan-3-yl)methyl]phenyl}-5-(trifluoromethyl)-1H-pyrazolo[3,4-c]pyridine-7-yl]ethan-1-one

### Step 1: 3-[(3-bromophenyl)(oxetan-3-yl)methyl]-4-methyl-4H-1,2,4-triazole (26a)

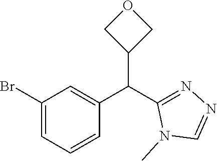 [SMILES: Cn1cnnc1C(c1cccc(Br)c1)C1COC1]

To a solution of 3-[(3-bromophenyl)methyl]-4-methyl-4H-1,2,4-triazole (23c) (500 mg, 1.98 mmol, 1.0 equiv) and 3-iodo-oxetane (547 mg, 2.97 mmol, 1.5 equiv) in DMF (10 mL) was added t-BuOK (336 mg, 2.99 mmol, 1.5 equiv, 1M in THF) dropwise at 0° C. under N₂. The reaction was stirred at 0° C. and allowed to slowly warm to room temperature overnight. The reaction mixture was directly purified by reverse phase preparative flash chromatography (ISCO, 21×250 mm column) to give 3-[(3-bromophenyl)(oxetan-3-yl)methyl]-4-methyl-4H-1,2,4-triazole (26a) (482 mg, 79%) as a glassy foam. LCMS (ESI+) 308.1 (M+H)⁺. ¹1H NMR (400 MHz, DMSO-d₆) δ 8.37 (s, 1H), 7.47 (ddd, J=7.6, 2.1, 1.4 Hz, 1H), 7.44 (t, J=1.8 Hz, 1H), 7.31 (t, J=7.6 Hz, 1H), 7.26 (dt, J=7.7, 1.5 Hz, 1H), 4.76 (d, J=11.0 Hz, 1H), 4.69 (dd, J=7.8, 6.2 Hz, 1H), 4.47 (dd, J=7.9, 6.0 Hz, 1H), 4.41 (t, J=6.3 Hz, 1H), 4.24 (t, J=6.3 Hz, 1H), 3.89-3.74 (m, 1H), 3.37 (s, 3H).

### Step 2: 4-methyl-3-{(oxetan-3-yl)[3-(4,4,5,5-tetramethyl-1,3,2-dioxaborolan-2-yl)phenyl]methyl}-4H-1,2,4-triazole (26b)

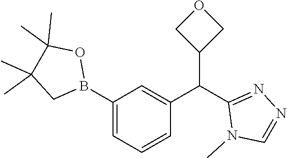

A solution of 3-[(3-bromophenyl)(oxetan-3-yl)methyl]-4-methyl-4H-1,2,4-triazole (26a) (482 mg, 1.56 mmol, 1.0 equiv), bis(pinacolato)diboron (794 mg, 3.13 mmol, 2.0 equiv), potassium acetate (307 mg, 3.13 mmol, 2.0 equiv) in dioxane (10 mL) was degassed and then [1,1′-bis(diphenylphosphino)ferrocene]dichloropalladium(II) (102 mg, 0.125 mmol, 0.08 equiv) was added and the reaction mixture was stirred at 80° C. for 16 h. The reaction mixture was cooled and concentrated under reduced pressure. The crude residue was purified by flash column chromatography (ISCO, 12 g SiO₂, DCM:EtOAc (1:1)/MeOH 0-15%) to give 4-methyl-3-{(oxetan-3-yl)[3-(4,4,5,5-tetramethyl-1,3,2-dioxaborolan-2-yl)phenyl]methyl}-4H-1,2,4-triazole (26b) (357 mg, 64%) as a light pink foam. LCMS (ESI+) 356.2 (M+H). ¹H NMR (400 MHz, DMSO-d₆) δ 8.35 (s, 1H), 7.56 (dt, J=7.3, 1.3 Hz, 1H), 7.49-7.41 (m, 2H), 7.36 (t, J=7.4 Hz, 1H), 4.76-4.68 (m, 2H), 4.44 (dd, J=7.9, 6.0 Hz, 1H), 4.38 (t, J=6.3 Hz, 1H), 4.23 (t, J=6.3 Hz, 1H), 3.85-3.74 (m, 1H), 3.33 (s, 3H), 1.28 (s, 12H).

### Step 3: 1-[3-{3-[(4-methyl-4H-1,2,4-triazol-3-yl)(oxetan-3-yl)methyl]phenyl}-5-(trifluoromethyl)-1H-pyrazolo[3,4-c]pyridin-7-yl]ethan-1-ol (26c)

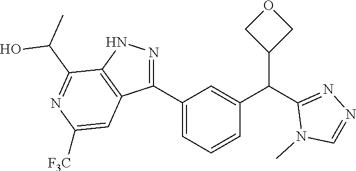 [SMILES: CC(O)c1nc(C(F)(F)F)cc2c(-c3cccc(C(c4nncn4C)C4COC4)c3)n[nH]c12]

To a solution of 1-(3-iodo-5-(trifluoromethyl)-1H-pyrazolo[3,4-c]pyridin-7-yl)ethan-1-ol (Intermediate 6′) (242 mg, 0.678 mmol) and 4-methyl-3-{(oxetan-3-yl)[3-(4,4,5,5-tetramethyl-1,3,2-dioxaborolan-2-yl)phenyl]methyl}-4H-1,2,4-triazole (26b) (242 mg, 0.681 mmol) in iso-propanol (8.0 mL, c=0.09 M) was added aqueous potassium phosphate (370 mg, 1.74 mmol, 1.74 mL, 1.0 M) and cataCXium® A Pd G3 (49.6 mg, 0.068 mmol). The mixture was degassed three times and heated at 110° C. overnight. The mixture was cooled to room temperature, filtered, and concentrated under reduced pressure onto silica gel. The residue was purified by flash column chromatography (ISCO, solvent 0-15% MeOH/1:1 DCM/EtOAc) to give 1-[3-{3-[(4-methyl-4H-1,2,4-triazol-3-yl)(oxetan-3-yl)methyl]phenyl}-5-(trifluoromethyl)-1H-pyrazolo[3,4-c]pyridin-7-yl]ethan-1-ol (26c) as a foamy solid (229 mg, 55% (of theoretical total 412 mg due to combining reactions for purification)). LCMS (ESI+) 459.1 (M+H)⁺. ¹H NMR (400 MHz, DMSO-d₆) δ 14.00 (s, 1H), 8.38 (s, 1H), 8.25 (d, J=1.8 Hz, 1H), 7.94 (br. dt, J=7.8, 1.3 Hz, 1H), 7.90 (br. t, J=1.9 Hz, 1H), 7.53 (t, J=7.7 Hz, 1H), 7.35-7.31 (m, 1H), 5.97 (dd, J=4.4, 1.1 Hz, 1H), 5.23-5.16 (m, 1H), 4.93 (d, J=11.0 Hz, 1H), 4.75 (dd, J=7.8, 6.2 Hz, 1H), 4.52 (d, J=7.2 Hz, 2H), 4.31 (t, J=6.3 Hz, 1H), 3.96-3.85 (m, 1H), 3.41 (s, 3H), 1.56 (d, J=6.6 Hz, 3H).

### Step 4: 1-[3-{3-[(4-methyl-4H-1,2,4-triazol-3-yl)(oxetan-3-yl)methyl]phenyl}-5-(trifluoromethyl)-1H-pyrazolo[3,4-c]pyridin-7-yl]ethan-1-one (Intermediate 26)

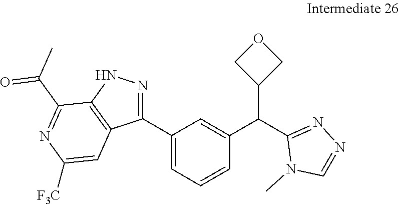 [SMILES: CC(=O)c1nc(C(F)(F)F)cc2c(-c3cccc(C(c4nncn4C)C4COC4)c3)n[nH]c12]

To a solution of 1-[3-{3-[(4-methyl-4H-1,2,4-triazol-3-yl)(oxetan-3-yl)methyl]phenyl}-5-(trifluoromethyl)-1H-pyrazolo[3,4-c]pyridin-7-yl]ethan-1-ol (26c) (117 mg, 0.386 mmol) in CHCl₃ (10 mL, c=0.04 M) was added MnO₂ (839 mg, 9.65 mmol). The resulting mixture was heated at 70° C. for 18 h. The mixture was cooled to RT, filtered through Celite, and washed with CH₂Cl₂. The filtrate was concentrated and taken up in CH₂Cl₂ and MTBE was added until cloudy. The mixture was allowed to stand at rt for 5 days, then the residue was triturated with MTBE and the solids were collected by filtration and dried in a vacuum oven at 50° C. for 3 h to give 1-[3-{3-[(4-methyl-4H-1,2,4-triazol-3-yl)(oxetan-3-yl)methyl]phenyl}-5-(trifluoromethyl)-1H-pyrazolo[3,4-c]pyridin-7-yl]ethan-1-one (Intermediate 26) as a pale orange solid (98 mg, 56%). LCMS (ESI+) 457.1 (M+H)⁺. ¹H NMR (400 MHz, DMSO-d₆) δ 14.46 (s, 1H), 8.69 (s, 1H), 8.38 (s, 1H), 8.00 (dt, J=7.8, 1.4 Hz, 1H), 7.95 (t, J=1.8 Hz, 1H), 7.56 (t, J=7.7 Hz, 1H), 7.37 (dt, J=7.8, 1.5 Hz, 1H), 4.94 (d, J=11.0 Hz, 1H), 4.75 (dd, J=7.8, 6.2 Hz, 1H), 4.52 (d, J=7.1 Hz, 2H), 4.31 (t, J=6.3 Hz, 1H), 3.97-3.85 (m, 1H), 3.42 (s, 3H), 2.79 (s, 3H).

### Preparation of Intermediate 27: 7-(1-bromoethyl)-3-{3-[1-(4-methyl-4H-1,2,4-triazol-3-yl)propyl]phenyl}-5-(trifluoromethyl)-1H-pyrazolo[3,4-c]pyridine

### Step 1: 1-[3-{3-[1-(4-methyl-4H-1,2,4-triazol-3-yl)propyl]phenyl}-5-(trifluoromethyl)-1H-pyrazolo[3,4-c]pyridin-7-yl]ethan-1-ol (27a)

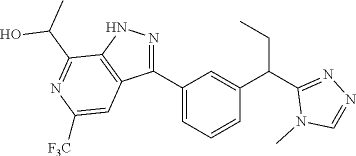 [SMILES: CCC(c1cccc(-c2n[nH]c3c(C(C)O)nc(C(F)(F)F)cc23)c1)c1nncn1C]

A solution of 1-(3-bromo-5-(trifluoromethyl)-1H-pyrazolo[3,4-c]pyridin-7-yl)ethan-1-ol (Intermediate 6) (630 mg, 2.03 mmol), 4-methyl-3-{1-[3-(4,4,5,5-tetramethyl-1,3,2-dioxaborolan-2-yl)phenyl]propyl}-4H-1,2,4-triazole (Intermediate 23) (731 mg, 2.23 mmol), [1,1′-bis(diphenylphosphino)ferrocene]dichloropalladium(II) (149 mg, 0.204 mmol), and CsF (926 mg, 6.10 mmol) in water (1.5 mL) and dioxane (7 mL) was degassed four times and heated at 90° C. overnight. Additional [1,1′-bis(diphenylphosphino)ferrocene]dichloropalladium(II) (70 mg, 0.096 mmol) was added and was degassed four times and heated at 90° C. overnight. The mixture was cooled to room temperature and concentrated under reduced pressure. The residue was purified by flash column chromatography (ISCO, 20 g SiO₂, MeOH/DCM 0-10%) to give 1-[3-{3-[1-(4-methyl-4H-1,2,4-triazol-3-yl)propyl]phenyl}-5-(trifluoromethyl)-1H-pyrazolo[3,4-c]pyridin-7-yl]ethan-1-ol (27a) as a brown solid (752 mg, 86%). LCMS (ESI+) 431.0 (M+H)⁺. ¹H NMR (400 MHz, DMSO-d₆) δ 14.02 (s, 1H), 8.36 (s, 1H), 8.29-8.24 (m, 1H), 7.90 (s, 2H), 7.57-7.50 (m, 1H), 7.36 (br d, J=7.5 Hz, 1H), 6.04-5.99 (m, 1H), 5.24-5.16 (m, 1H), 4.32-4.24 (m, 1H), 3.46 (s, 3H), 2.32-2.23 (m, 1H), 2.10-2.01 (m, 1H), 1.60-1.53 (m, 3H), 0.92 (s, 3H).

### Step 2: 7-(1-bromoethyl)-3-{3-[1-(4-methyl-4H-1,2,4-triazol-3-yl)propyl]phenyl}-5-(trifluoromethyl)-1H-pyrazolo[3,4-c]pyridine (Intermediate 27)

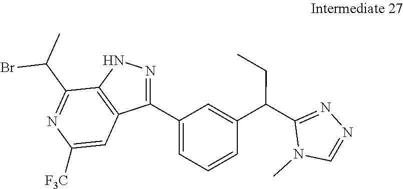 [SMILES: CCC(c1cccc(-c2n[nH]c3c(C(C)Br)nc(C(F)(F)F)cc23)c1)c1nncn1C]

Triphenylphosphine dibromide (2720 mg, 6.44 mmol) was added to a solution of 1-[3-{3-[1-(4-methyl-4H-1,2,4-triazol-3-yl)propyl]phenyl}-5-(trifluoromethyl)-1H-pyrazolo[3,4-c]pyridin-7-yl]ethan-1-ol (27a) (693 mg, 1.61 mmol) in DCM (12 mL) at 0° C. The mixture was stirred at RT for 2 h. The mixture was used directly without further purification. LCMS (ESI+) 492.9 (M+H)⁺.

### Preparation of Intermediate 28: 7-chloro-3-(3-{3-[(4-methyl-4H-1,2,4-triazol-3-yl)methyl]oxetan-3-yl}phenyl)-5-(trifluoromethyl)-1H-pyrazolo[3,4-c]pyridine

### Step 1: 7-chloro-3-iodo-5-(trifluoromethyl)-1H-pyrazolo[3,4-c]pyridine (28a)

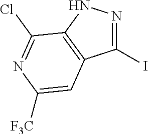 [SMILES: FC(F)(F)c1cc2c(I)n[nH]c2c(Cl)n1]

To a solution of 7-chloro-5-(trifluoromethyl)-1H-pyrazolo[3,4-c]pyridine (500 mg, 2.26 mmol) in DMF (10 mL) was added KOH (633 mg, 11.3 mmol) and iodine (2.29 g, 9.03 mmol) and the mixture was stirred at 25° C. for 16 h. The mixture was diluted with EtOAc (30 mL), washed with saturated aqueous Na₂SO₃ (20 mL), brine (20 mL), dried over Na₂SO₄, filtered, and concentrated under reduced pressure. The residue was purified by flash column chromatography (ISCO, 20 g SiO₂, EtOAc/pet ether 0-30%) to give 7-chloro-3-iodo-5-(trifluoromethyl)-1H-pyrazolo[3,4-c]pyridine (28a) as a white solid (630 mg, 80%). LCMS (ESI+) 347.8 (M+H)⁺. ¹H NMR (400 MHz, CDCl₃) 11.02 (brs, 1H), 7.83 (s, 1H).

### Step 2: 7-chloro-3-(3-{3-[(4-methyl-4H-1,2,4-triazol-3-yl)methyl]oxetan-3-yl}phenyl)-5-(trifluoromethyl)-1H-pyrazolo[3,4-c]pyridine (Intermediate 28)

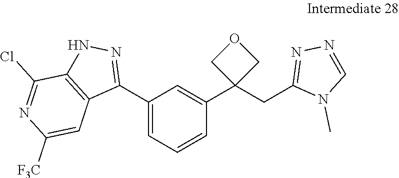 [SMILES: Cn1cnnc1CC1(c2cccc(-c3n[nH]c4c(Cl)nc(C(F)(F)F)cc34)c2)COC1]

A solution of 7-chloro-3-iodo-5-(trifluoromethyl)-1H-pyrazolo[3,4-c]pyridine (28a) (820 mg, 2.36 mmol), 4-methyl-3-({3-[3-(4,4,5,5-tetramethyl-1,3,2-dioxaborolan-2-yl)phenyl]oxetan-3-yl}methyl)-4H-1,2,4-triazole (Intermediate 2) (922 mg, 2.60 mmol), [1,1′-bis(diphenylphosphino)ferrocene]dichloropalladium(II) (173 mg, 0.236 mmol), and Na₂CO₃ (750 mg, 7.08 mmol) in water (3 mL) and dioxane (16 mL) was degassed five times and heated at 100° C. for 18 h. The mixture was cooled to room temperature and diluted with EtOAc (10 mL) and water (5 mL). The layers were separated and the aqueous layer was extracted with EtOAc (6×5 mL). The combined organic layers were washed with brine (30 mL), dried over Na₂SO₄, filtered, and concentrated under reduced pressure. The residue was purified by flash column chromatography (ISCO, 20 g SiO₂, MeOH/DCM 0-12%) to give 7-chloro-3-(3-{3-[(4-methyl-4H-1,2,4-triazol-3-yl)methyl]oxetan-3-yl}phenyl)-5-(trifluoromethyl)-1H-pyrazolo[3,4-c]pyridine (Intermediate 28) as a brown solid (628 mg, 59%). LCMS (ESI+) 449.3 (M+H)⁺. ¹H NMR (400 MHz, DMSO-d₆) δ 14.88 (br s, 1H), 8.42 (s, 1H), 8.19 (s, 1H), 7.92 (br d, J=7.2 Hz, 1H), 7.63 (s, 1H), 7.49 (br t, J=7.6 Hz, 1H), 7.13 (br d, J=7.6 Hz, 1H), 5.06-4.91 (m, 4H), 3.58 (s, 2H), 2.99 (s, 3H).

### Preparation of Intermediate 29: 7-bromo-3-{3-[(R)-cyclobutyl(4-methyl-4H-1,2,4-triazol-3-yl)methyl]phenyl}-5-(trifluoromethyl)-1H-pyrazolo[3,4-c]pyridine

### Step 1: 7-bromo-5-(trifluoromethyl)-1H-pyrazolo[3,4-c]pyridine (29a)

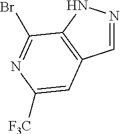 [SMILES: FC(F)(F)c1cc2cn[nH]c2c(Br)n1]

To a solution of 2-bromo-4-methyl-6-(trifluoromethyl)pyridin-3-amine (10.0 g, 39.2 mmol) in acetic acid (130 mL) was added sodium nitrite (5.41 g, 78.4 mmol) at rt portionwise and the solution was stirred at rt for 3 h. The mixture was concentrated under reduced pressure. The residue was dissolved in water (150 mL) and EtOAc (150 mL) and basified with solid Na₂CO₃. The layers were separated, and the aqueous layer was extracted with EtOAc (3×100 mL). The combined organic layers were washed with brine (200 mL), dried over Na₂SO₄, filtered, and concentrated under reduced pressure. The crude residue was purified by flash column chromatography (Isco, 120 g SiO₂, pet ether/EtOAc 0-20%) to afford a yellow solid which was slurried with pet ether/EtOAc (20 mL/5 mL) for 30 min. The mixture was filtered to yield 7-bromo-5-(trifluoromethyl)-1H-pyrazolo[3,4-c]pyridine (29a) as a light yellow solid (2.7 g, 26%). LCMS (ESI+) 267.8 (M+H)⁺. ¹H NMR (400 MHz, CDCl₃) δ 10.89 (br s, 1H), 8.40 (s, 1H), 8.11 (s, 1H).

### Step 2: 7-bromo-3-iodo-5-(trifluoromethyl)-1H-pyrazolo[3,4-c]pyridine (29b)

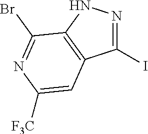 [SMILES: FC(F)(F)c1cc2c(I)n[nH]c2c(Br)n1]

To a mixture of 7-bromo-5-(trifluoromethyl)-1H-pyrazolo[3,4-c]pyridine (29a) (5.00 g, 18.8 mmol) in DMF (120 mL) was added potassium carbonate (5.27 g, 94.0 mmol) and iodine (19.1 g, 75.2 mmol). The mixture was stirred at rt for 3 h, then diluted with EtOAc (100 mL), washed with saturated Na₂SO₃, washed with brine (5×200 mL), dried over Na₂SO₄, filtered and concentrated to give 7-bromo-3-iodo-5-(trifluoromethyl)-1H-pyrazolo[3,4-c]pyridine (29b) as a yellow solid which was carried forward without further purification (7.1 g, 97%). LCMS (ESI+) 393.7 (M+H)⁺. ¹H NMR (400 MHz, DMSO-d₆) δ 15.05 (br s, 1H), 8.02 (s, 1H).

### Step 3: 7-bromo-3-iodo-5-(trifluoromethyl)-1-{[2-(trimethylsilyl)ethoxy]methyl}-1H-pyrazolo[3,4-c]pyridine (29c)

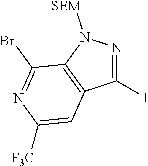 [SMILES: C[Si](C)(C)CCOCn1nc(I)c2cc(C(F)(F)F)nc(Br)c21]

To a solution of 7-bromo-3-iodo-5-(trifluoromethyl)-1H-pyrazolo[3,4-c]pyridine (29b) (6.10 g, 15.6 mmol) in THF (90 mL) was added NaH (0.25 g, 31.1 mmol) at 0° C. and the reaction was stirred at 0° C. for 30 min. Then, 2-(trimethylsilyl)ethoxymethyl chloride (3.89 g, 23.3 mmol) was added at 0° C. and the reaction was warmed to 15° C. and stirred for 2 h. The reaction mixture was diluted with EtOAc (50 mL) and water (50 mL) and the layers were separated. The organic layer was washed with brine (50 mL), dried over Na₂SO₄, filtered and concentrated under reduced pressure. The material was purified via flash column chromatography (ISCO, 30 g SiO₂, 0-20% pet ether/EtOAc) to afford 7-bromo-3-iodo-5-(trifluoromethyl)-1-{[2-(trimethylsilyl)ethoxy]methyl}-1H-pyrazolo[3,4-c]pyridine (29c) as a colorless oil (6.00 g, 74%). LCMS (ESI+) 523.8 (M+H)⁺. ¹H NMR (400 MHz, DMSO-d₆) δ 8.08 (s, 1H), 6.07 (s, 2H), 3.65-3.55 (m, 2H), 0.83-0.74 (m, 2H), −0.11 (s, 9H).

### Step 4: 7-bromo-3-{3-[(R)-cyclobutyl(4-methyl-4H-1,2,4-triazol-3-yl)methyl]phenyl}-5-(trifluoromethyl)-1-{[2-(trimethylsilyl)ethoxy]methyl}-1H-pyrazolo[3,4-c]pyridine (29d)

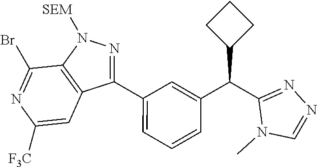 [SMILES: Cn1cnnc1[C@@H](c1cccc(-c2nn(COCC[Si](C)(C)C)c3c(Br)nc(C(F)(F)F)cc23)c1)C1CCC1]

To a solution of 7-bromo-3-iodo-5-(trifluoromethyl)-1-{[2-(trimethylsilyl)ethoxy]methyl}-1H-pyrazolo[3,4-c]pyridine (29c) (3.00 g, 5.75 mmol), (R)-3-(cyclobutyl(3-(4,4,5,5-tetramethyl-1,3,2-dioxaborolan-2-yl)phenyl)-methyl)-4-methyl-4H-1,2,4-triazole (Intermediate 1′) (2.44 g, 6.89 mmol), and CsF (2.62 g, 17.2 mmol) in dioxane (75 mL) and water (15 mL) was added 1,1′-bis(diphenylphosphino)ferrocene]dichloropalladium(II) (420 mg, 0.575 mmol). The mixture was bubbled with N₂ and stirred at 90° C. for 16 h. Additional (R)-3-(cyclobutyl(3-(4,4,5,5-tetramethyl-1,3,2-dioxaborolan-2-yl)phenyl)-methyl)-4-methyl-4H-1,2,4-triazole (Intermediate 1′) (203 mg, 0.575 mmol) and 1,1′-bis(diphenylphosphino)ferrocene]dichloropalladium(II) (105 mg, 0.144 mmol) were added and the mixture was bubbled with N₂ and stirred at 90° C. for 7 h. The mixture was then cooled to room temperature, diluted with EtOAc (50 mL) and washed with water (100 mL), dried over Na₂SO₄, and concentrated under reduced pressure. The crude product was purified by flash column chromatography (ISCO, 80 g SiO₂, MeOH/DCM 0-10%) to give 7-bromo-3-{3-[(R)-cyclobutyl(4-methyl-4H-1,2,4-triazol-3-yl)methyl]phenyl}-5-(trifluoromethyl)-1-{[2-(trimethylsilyl)ethoxy]methyl}-1H-pyrazolo[3,4-c]pyridine (29d) as a brown gum (3.10 g, 87%). LCMS (ESI+) 623.1 (M+H)⁺. ¹H NMR (400 MHz, DMSO-d₆) δ 8.44 (s, 1H), 8.34 (s, 1H), 8.00-7.82 (m, 2H), 7.54 (br t, J=8.0 Hz, 1H), 7.41 (br s, 1H), 6.16 (s, 2H), 3.65 (br t, J=7.9 Hz, 2H), 3.45 (s, 3H), 3.39-3.36 (m, 1H), 2.09 (br s, 1H), 1.94-1.64 (m, 6H), 0.83 (br t, J=7.8 Hz, 2H), −0.12 (s, 9H).

### Step 5: 7-bromo-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 29)

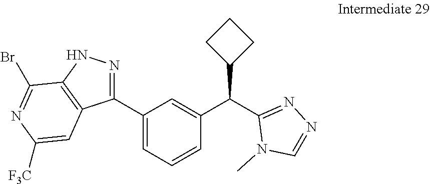 [SMILES: Cn1cnnc1[C@@H](c1cccc(-c2n[nH]c3c(Br)nc(C(F)(F)F)cc23)c1)C1CCC1]

To a solution of 7-bromo-3-{3-[(R)-cyclobutyl(4-methyl-4H-1,2,4-triazol-3-yl)methyl]phenyl}-5-(trifluoromethyl)-1-{[2-(trimethylsilyl)ethoxy]methyl}-1H-pyrazolo[3,4-c]pyridine (29d) in DCM (150 mL) was added TFA (10 mL). The mixture was stirred at 15° C. for 1.5 h. The solution was concentrated under reduced pressure. Then NH₃ and MeOH were added and the reaction mixture was stirred. The mixture was concentrated under reduced pressure and purified via flash column chromatography (ISCO, 40 g SiO₂, 0-5% DCM/MeOH) to afford 7-bromo-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 29) as a grey solid (1.30 g, 55%). LCMS (ESI+) 491.0 (M+H)⁺. ¹H NMR (400 MHz, DMSO-d₆) δ 14.77 (s, 1H), 8.44 (s, 1H), 8.35 (s, 1H), 7.91 (br s, 2H), 7.51 (br t, J=7.7 Hz, 1H), 7.36 (br d, J=7.3 Hz, 1H), 4.38 (d, J=10.5 Hz, 1H), 3.45 (s, 3H), 2.09 (br s, 1H), 1.88-1.66 (m, 6H).

### Preparation of Intermediate 30: 7-chloro-3-{3-[(R)-cyclobutyl(4-methyl-4H-1,2,4-triazol-3-yl)methyl]phenyl}-5-(trifluoromethyl)-1H-pyrazolo[3,4-c]pyridine

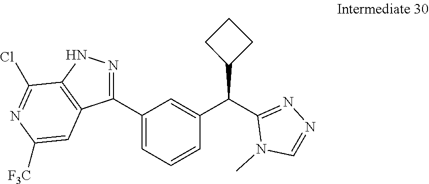 [SMILES: Cn1cnnc1[C@@H](c1cccc(-c2n[nH]c3c(Cl)nc(C(F)(F)F)cc23)c1)C1CCC1]

To a solution of 7-chloro-3-iodo-5-(trifluoromethyl)-1H-pyrazolo[3,4-c]pyridine (28a) (197 mg, 0.567 mmol) and (R)-3-(cyclobutyl(3-(4,4,5,5-tetramethyl-1,3,2-dioxaborolan-2-yl)phenyl)-methyl)-4-methyl-4H-1,2,4-triazole (Intermediate 1′) (220 mg, 0.624 mmol) in iso-propanol (3.78 mL, c=0.15 M) was added aqueous potassium phosphate (301 mg, 1.42 mmol, 1.42 mL, 1.0 M) and cataCXium® A Pd G3 (41.3 mg, 0.0567 mmol). The mixture was degassed three times and heated at 110° C. overnight. The mixture was cooled to room temperature, diluted with brine (10 mL) and extracted with DCM (2×10 mL). The combined organic layers were dried over sodium sulfate and concentrated. The residue was purified by flash column chromatography (ISCO, solvent 0-10% MeOH/1:1 DCM/EtOAc) to give 7-chloro-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 30) as a pale brown foam (250 mg, 99%). LCMS (APCI+) 447.0. ¹H NMR (400 MHz, CD₃OD) δ 8.38 (s, 1H), 8.27 (s, 1H), 7.90 (d, 1H, J=7.8 Hz), 7.87 (s, 1H), 7.56 (t, 1H, J=7.8 Hz), 7.39 (d, 1H, J=7.8 Hz), 4.35 (d, 1H, J=10.9 Hz), 3.55 (s, 3H), 2.29 (tdd, 1H, J=3.7, 7.4, 11.1 Hz), 2.00-1.90 (m, 4H), 1.90-1.73 (m, 2H).

### Preparation of Intermediate 31: tert-butyl 3-bromo-5-(trifluoromethyl)-1H-pyrazolo[3,4-c]pyridine-1-carboxylate

### Step 1: 3-bromo-5-(trifluoromethyl)-1H-pyrazolo[3,4-c]pyridine (31a)

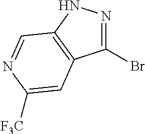 [SMILES: FC(F)(F)c1cc2c(Br)n[nH]c2cn1]

To a mixture of 5-(trifluoromethyl)-1H-pyrazolo[3,4-c]pyridine (5.30 g, 28.3 mmol) in MeCN (60 mL) was added N-bromosuccinimide (5.55 g, 31.2 mmol) in portions. The mixture was stirred at rt for 16 h and then concentrated under reduced pressure. The crude residue was purified via flash column chromatography (ISCO, 80 g SiO₂, 0-30% pet ether/EtOAc) to give 3-bromo-5-(trifluoromethyl)-1H-pyrazolo[3,4-c]pyridine (31a) (5.10 g, 68%) as a yellow solid. ¹H NMR (400 MHz, CDCl₃) δ 11.13 (br s, 1H), 9.15 (s, 1H), 8.05 (s, 1H).

### Step 2: tert-butyl 3-bromo-5-(trifluoromethyl)-1H-pyrazolo[3,4-c]pyridine-1-carboxylate (Intermediate 31)

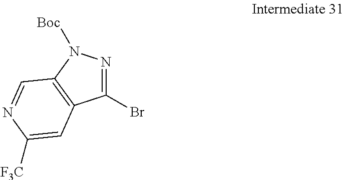 [SMILES: CC(C)(C)OC(=O)n1nc(Br)c2cc(C(F)(F)F)ncc21]

To a solution of 3-bromo-5-(trifluoromethyl)-1H-pyrazolo[3,4-c]pyridine (31a) (5.00 g, 18.8 mmol) in MeCN (100 mL) was added triethylamine (2.28 g, 22.6 mmol) and Boc anhydride (4.72 g, 21.6 mmol), and the reaction was stirred at rt for 16 h. The reaction mixture was concentrated under reduced pressure and purified via flash column chromatography (ISCO, 80 g SiO₂, 0-20% pet ether/EtOAc) to give tert-butyl 3-bromo-5-(trifluoromethyl)-1H-pyrazolo[3,4-c]pyridine-1-carboxylate (Intermediate 31) (6.34 g, 92%) as a white solid. ¹H NMR (400 MHz, CDCl₃) δ 9.63 (s, 1H), 8.00 (s, 1H), 1.76 (s, 9H).

### Preparation of Intermediate 32: tert-butyl {(1S)-1-[3-bromo-5-(trifluoromethyl)-1H-pyrazolo[3,4-c]pyridin-7-yl]ethyl}carbamate

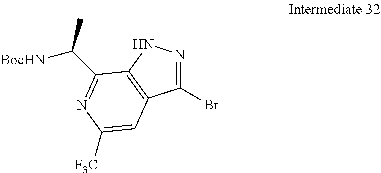 [SMILES: C[C@H](NC(=O)OC(C)(C)C)c1nc(C(F)(F)F)cc2c(Br)n[nH]c12]

To a 3-necked 1 L flask equipped with a condenser and an internal thermometer was added 1-[3-bromo-5-(trifluoromethyl)-1H-pyrazolo[3,4-c]pyridin-7-yl]ethan-1-ol (Intermediate 6) (22.7 g, 73 mmol, 1 equiv), (S,S)-2-methylpropane-2-sulfinamide (13.3 g, 110 mmol, 1.5 equiv), catalyst Ru-Macho (888 mg, 1.46 mmol, 0.02 equiv) and toluene (366 mL, 0.2 M). The flask was placed under nitrogen atmosphere. LHMDS (1 M in toluene, 110 mL, 110 mmol, 1.5 equiv) was added over 5 min to reach a final concentration of 0.15 M. The mixture was heated in an oil bath at 125° C. (bath temperature) overnight. Then, the volatiles were removed under reduced pressure.

To the above residue was added methanol (300 mL) and aqueous HCl (6 M, 122 mL, 10 equiv) and stirred for 3 h. The volatiles were removed under reduced pressure.

To the above residue was added aqueous K₂CO₃ (2 M, 100 mL), to reach pH 8. Ethyl acetate (300 mL) was added followed by Boc anhydride (17.6 g, 80.5 mmol, 1.1 equiv). The mixture was stirred at room temperature overnight, then water (100 mL) was added, and the product was extracted with ethyl acetate (3×250 mL). The combined organic layer was dried over Na₂SO₄, filtered, and evaporated to give a solid gum (34.2 g). The crude material was purified on silica (20% ethyl acetate-80% heptane) to give tert-butyl {(1S)-1-[3-bromo-5-(trifluoromethyl)-1H-pyrazolo[3,4-c]pyridin-7-yl]ethyl}-carbamate (Intermediate 32) as a light yellow solid (19.2 g, 64%, >99% ee). LCMS (ESI+) 409.0/411.0 (M+H)⁺. ¹H NMR (400 MHz, CDCl₃) δ 12.38 (br s, 1H), 7.89 (s, 1H), 5.46-5.31 (m, 1H), 5.13 (br d, J=8.1 Hz, 1H), 1.79 (d, J=6.8 Hz, 3H), 1.47 (s, 9H). The bis-Boc side product (5.1 g) which was isolated from the above column was treated with ammonia (7 N in methanol, 10 mL) for 1 hour at room temperature. Evaporation and purification as above gave an additional crop of the title product (3.4 g, 11%, total=75%).

### Preparation of Intermediate 33: tert-butyl {(1R)-1-[3-bromo-5-(trifluoromethyl)-1H-pyrazolo[3,4-c]pyridin-7-yl]ethyl}carbamate

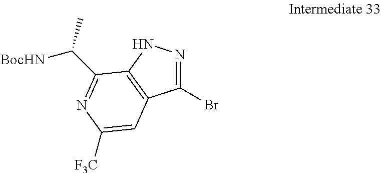 [SMILES: C[C@@H](NC(=O)OC(C)(C)C)c1nc(C(F)(F)F)cc2c(Br)n[nH]c12]

To a mixture of 1-[3-bromo-5-(trifluoromethyl)-1H-pyrazolo[3,4-c]pyridin-7-yl]ethan-1-ol (Intermediate 6) (1.00 g, 3.22 mmol, 1 equiv), (R,R)-2-methylpropane-2-sulfinamide (586 mg, 4.40 mmol, 1.5 equiv), catalyst Ru-Macho (39.2 mg, 0.065 mmol, 0.02 equiv) and toluene (16.1 mL, 0.2 M) was added LHMDS (1 M in toluene, 4.84 mL, 4.84 mmol, 1.5 equiv). The mixture was heated at 125° C. (bath temperature) for 24 h. Then, the volatiles were removed under reduced pressure.

To the above residue was added methanol (12 mL) and aqueous HCl (6 M, 5.38 mL, 10 equiv) and stirred for 3 h. The volatiles were removed under reduced pressure.

To the above residue was added aqueous K₂CO₃ (2 M, 4 mL), to reach pH 8. Ethyl acetate (30 mL) was added followed by Boc anhydride (774 mg, 3.55 mmol, 1.1 equiv). The mixture was stirred at room temperature overnight, then the layers were separated, and the organic layer was dried over Na₂SO₄, filtered, and concentrated under reduced pressure. The crude material was purified on silica (Isco, heptane/EtOAc 0-100%) to give tert-butyl {(1R)-1-[3-bromo-5-(trifluoromethyl)-1H-pyrazolo[3,4-c]pyridin-7-yl]ethyl}-carbamate (Intermediate 33) as a brown solid (720 mg, 55%). LCMS (APCI+) 309.0/311.0 (M+H-Boc)⁺. ¹H NMR (400 MHz, CDCl₃) δ 12.47 (br s, 1H), 7.89 (s, 1H), 5.50-5.34 (m, 1H), 1.77 (d, J=6.9 Hz, 3H), 1.47 (s, 9H).

### Preparation of Intermediate 34: 2-[3-bromo-5-(trifluoromethyl)-1H-pyrazolo[3,4-c]pyridin-7-yl]propan-1-ol

### Step 1: 2-[5-(trifluoromethyl)-1H-pyrazolo[3,4-c]pyridin-7-yl]prop-2-en-1-ol (34a)

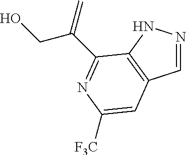 [SMILES: C=C(CO)c1nc(C(F)(F)F)cc2cn[nH]c12]

A mixture of 7-bromo-5-(trifluoromethyl)-1H-pyrazolo[3,4-c]pyridine (29a) (5.00 g, 18.8 mmol), tert-butyl(dimethyl){[2-(4,4,5,5-tetramethyl-1,3,2-dioxaborolan-2-yl)prop-2-en-1-yl]oxy}silane (6.73 g, 22.6 mmol), CsF (5.71 g, 37.6 mmol), and 1′-bis(diphenylphosphino)ferrocene]dichloropalladium(II) (1.38 g, 1.88 mmol) in dioxane (100 mL) and water (20 mL) was degassed with N₂ and stirred at 100° C. for 16 h. The mixture was then cooled to room temperature, diluted with EtOAc (50 mL) and water (50 mL). The layers were separated and the aqueous layer was extracted with EtOAc (50 mL). The combined organic layers were washed with brine, dried over Na₂SO₄, filtered, and concentrated under reduced pressure. The crude product was purified by flash column chromatography (ISCO, 80 g SiO₂, MeOH/DCM 0-10%) followed by a second purification via flash column chromatography (ISCO, 40 g SiO₂, pet ether/EtOAc 0-100%) to give 2-[5-(trifluoromethyl)-1H-pyrazolo[3,4-c]pyridin-7-yl]prop-2-en-1-ol (34a) as a yellow solid (3.53 g, 77%). LCMS (ESI+) 244.0 (M+H)⁺.

### Step 2: 2-[5-(trifluoromethyl)-1H-pyrazolo[3,4-c]pyridin-7-yl]propan-1-ol (34b)

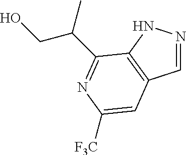 [SMILES: CC(CO)c1nc(C(F)(F)F)cc2cn[nH]c12]

To a solution of 2-[5-(trifluoromethyl)-1H-pyrazolo[3,4-c]pyridin-7-yl]prop-2-en-1-ol (34a) (3.53 g, 14.5 mmol) in MeOH (60 mL) was added Pd/C (10%, 1.00 g, 9.40 mmol). The reaction was stirred under a H₂ balloon at rt for 2 h. The reaction was concentrated under reduced pressure and purified via flash column chromatography (ISCO, 40 g SiO₂, pet ether/EtOAc 0-40%) to give 2-[5-(trifluoromethyl)-1H-pyrazolo[3,4-c]pyridin-7-yl]propan-1-ol (34b) (1.95 g, 55%) as a yellow gum.

### Step 3: 2-[3-bromo-5-(trifluoromethyl)-1H-pyrazolo[3,4-c]pyridin-7-yl]propan-1-ol (Intermediate 34)

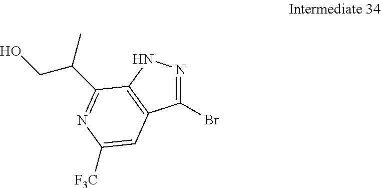 [SMILES: CC(CO)c1nc(C(F)(F)F)cc2c(Br)n[nH]c12]

To a mixture of 2-[5-(trifluoromethyl)-1H-pyrazolo[3,4-c]pyridin-7-yl]propan-1-ol (34b) (1.95 g, 7.95 mmol) in MeCN (30 mL) was added N-bromosuccinimide (1.70 g, 9.54 mmol). The mixture was stirred at 30° C. for 16 h and then concentrated under reduced pressure. The crude residue was purified via flash column chromatography (ISCO, 40 g SiO₂, 0-40% pet ether/EtOAc) to give 2-[3-bromo-5-(trifluoromethyl)-1H-pyrazolo[3,4-c]pyridin-7-yl]propan-1-ol (Intermediate 34) (1.90 g, 74%) as a white solid. ¹H NMR (400 MHz, DMSO-d₆) δ 14.53 (s, 1H), 7.94 (s, 1H), 4.75 (br s, 1H), 3.79 (br d, J=8.0 Hz, 1H), 3.76-3.61 (m, 2H), 1.31 (d, J=6.8 Hz, 3H).

### Preparation of Intermediate 35: tert-butyl {2-[3-bromo-5-(trifluoromethyl)-1H-pyrazolo[3,4-c]pyridin-7-yl]ethyl}carbamate

### Step 1: 7-chloro-5-(trifluoromethyl)-1-{[2-(trimethylsilyl)ethoxy]methyl}-1H-pyrazolo[3,4-c]pyridine (35a)

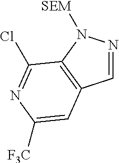 [SMILES: C[Si](C)(C)CCOCn1ncc2cc(C(F)(F)F)nc(Cl)c21]

To a suspension of 7-chloro-5-(trifluoromethyl)-1H-pyrazolo[3,4-c]pyridine (200 mg, 0.90 mmol) in dimethyl formamide (4.51 mL, 0.2 M) was added sodium hydride (60% in mineral oil, 57.8 mg, 1.44 mmol) and allowed to stir for 1.5 h. Then, (2-(chloromethoxy)ethyl)trimethylsilane (196 mg, 1.17 mmol, 0.208 mL) was added dropwise and allowed to stir for 1 h. The reaction was quenched via dropwise addition of water. The layers were separated and the aqueous layer was extracted with EtOAc (3×). The combined organic layers were dried over MgSO₄, filtered, and concentrated under reduced pressure. The crude residue was purified via flash column chromatography (ISCO, 12 g SiO₂, 0-50% Heptane:EtOAc) to afford 7-chloro-5-(trifluoromethyl)-1-{[2-(trimethylsilyl)ethoxy]methyl}-1H-pyrazolo[3,4-c]pyridine (35a) as a colorless oil (172 mg, 54%). LCMS (APCI+) 352.1 (M+H)⁺. ¹H NMR (400 MHz, CDCl₃) δ 8.28 (s, 1H), 8.07 (s, 1H), 6.15 (s, 2H), 3.70-3.63 (m, 2H), 1.59 (s, 2H), 0.01-−0.01 (m, 9H).

### Step 2: tert-butyl {2-[5-(trifluoromethyl)-1-{[2-(trimethylsilyl)ethoxy]methyl}-1H-pyrazolo[3,4-c]pyridin-7-yl]ethyl}carbamate (35b)

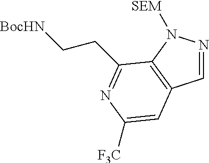 [SMILES: CC(C)(C)OC(=O)NCCc1nc(C(F)(F)F)cc2cnn(COCC[Si](C)(C)C)c12]

A mixture of 7-chloro-5-(trifluoromethyl)-1-{[2-(trimethylsilyl)ethoxy]methyl}-1H-pyrazolo[3,4-c]pyridine (35a) (50 mg, 0.14 mmol), potassium 2-(Boc-aminoethyl)trifluoroborate (35.7 mg, 0.142 mmol), cesium carbonate (139 mg, 0.426 mmol), palladium(II) acetate (6.38 mg, 0.0284 mmol), CataCXium A (10.2 mg, 0.284 mmol), toluene (0.474 mL, 0.3 M) and water (0.150 mL, 3.0 M). The reaction mixture was stirred at 80° C. for 18 h. The reaction mixture was cooled to rt, diluted with EtOAc and water, and the layers were separated. The aqueous layer was extracted with EtOAc (3×). Then the combined organic layers were dried over MgSO₄, filtered, and concentrated under reduced pressure. The material was purified via flash column chromatography (ISCO, 4 g SiO₂, 0-50% Heptane/EtOAc) to afford tert-butyl {2-[5-(trifluoromethyl)-1-{[2-(trimethylsilyl)ethoxy]methyl}-1H-pyrazolo[3,4-c]pyridin-7-yl]ethyl}carbamate (35b) as a yellow oil (22.7 mg, 35%). LCMS (APCI+) 361.1 (M+H-Boc)⁺. ¹H NMR (400 MHz, CDCl₃) δ 8.19 (s, 1H), 7.99 (s, 1H), 6.00 (s, 2H), 5.45 (br s, 1H), 3.87-3.80 (m, 2H), 3.64-3.55 (m, 4H), 1.48 (s, 9H), 0.96-0.92 (m, 2H), 0.00 (s, 9H).

### Step 3: tert-butyl {2-[5-(trifluoromethyl)-1H-pyrazolo[3,4-c]pyridin-7-yl]ethyl}carbamate (35c)

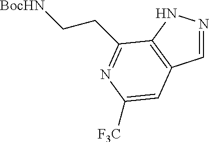 [SMILES: CC(C)(C)OC(=O)NCCc1nc(C(F)(F)F)cc2cn[nH]c12]

To a solution of tert-butyl {2-[5-(trifluoromethyl)-1-{[2-(trimethylsilyl)ethoxy]methyl}-1H-pyrazolo[3,4-c]pyridin-7-yl]ethyl}carbamate (35b) (155 mg, 0.337 mmol) in THF (1.68 mL, 0.2 M) was added tetrabutylammonium fluoride solution (440 mg, 1.68 mmol, 1.68 mL, 1 M). The vial was stirred at 70° C. for 20 h. The reaction was cooled diluted with EtOAc and saturated aqueous NaHCO₃. The layers were separated and the aqueous layer was extracted with EtOAc (2×). The combined organic layers were dried over MgSO₄, filtered, and concentrated under reduced pressure. The material was then purified via flash column chromatography (ISCO, 12 g SiO₂, 0-100% Heptane/EtOAc) to afford tert-butyl {2-[5-(trifluoromethyl)-1H-pyrazolo[3,4-c]pyridin-7-yl]ethyl}carbamate (35c) as a yellow solid (49 mg, 44%). ¹H NMR (400 MHz, CDCl₃) δ 8.26 (s, 1H), 8.02-7.99 (m, 1H), 5.27-5.16 (m, 1H), 3.63-3.54 (m, 2H), 3.51-3.45 (m, 2H), 1.55-1.54 (m, 9H).

### Step 4: tert-butyl {2-[3-bromo-5-(trifluoromethyl)-1H-pyrazolo[3,4-c]pyridin-7-yl]ethyl}carbamate (Intermediate 35)

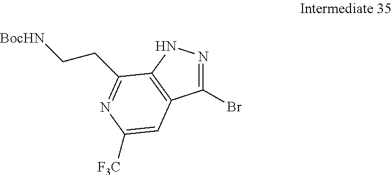 [SMILES: CC(C)(C)OC(=O)NCCc1nc(C(F)(F)F)cc2c(Br)n[nH]c12]

To a solution of tert-butyl {2-[5-(trifluoromethyl)-1H-pyrazolo[3,4-c]pyridin-7-yl]ethyl}carbamate (35c) (49 mg, 0.15 mmol) in 1,1,1,3,3,3-hexafluoropropan-2-ol (1.48 mL, 0.1 M) was added N-bromosuccinimide (31.7, 0.178 mmol), and the mixture was stirred at 40° C. overnight. The mixture was quenched with saturated Na₂S₂O₃ (15 mL) and extracted with DCM (2×15 mL). The combined organic layers were dried over Na₂SO₄, filtered, and concentrated under reduced pressure. The crude material was purified by flash column chromatography (ISCO, 0-100% EtOAc/heptane) to give tert-butyl {2-[3-bromo-5-(trifluoromethyl)-1H-pyrazolo[3,4-c]pyridin-7-yl]ethyl}carbamate (Intermediate 35) as a white solid (41.0 mg, 68%). LCMS (APCI+) 410.1 (M+H)⁺. ¹H NMR (400 MHz, CDCl₃) δ 13.22 (br d, J=0.9 Hz, 1H), 7.88 (s, 1H), 5.30-5.12 (m, 1H), 3.68-3.51 (m, 2H), 3.51-3.40 (m, 2H), 1.53 (s, 9H).

### Preparation of Intermediate 36: tert-butyl {2-[3-bromo-5-(trifluoromethyl)-1H-pyrazolo[3,4-c]pyridin-7-yl]propyl}carbamate

### Step 1: 7-bromo-5-(trifluoromethyl)-1-{[2-(trimethylsilyl)ethoxy]methyl}-1H-pyrazolo[3,4-c]pyridine (36a)

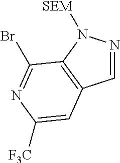 [SMILES: C[Si](C)(C)CCOCn1ncc2cc(C(F)(F)F)nc(Br)c21]

To a solution of 7-bromo-5-(trifluoromethyl)-1H-pyrazolo[3,4-c]pyridine (29a) (1168 mg, 4.39 mmol) in DMF (22.0 mL, 0.2 M) was added sodium hydride (281 mg, 7.03 mmol) at 0° C. The reaction mixture was then stirred at room temperature for 1 h. Then, (2-(chloromethoxy)ethyl)trimethylsilane (952 mg, 5.71 mmol, 1.01 mL) was added dropwise at 0° C. and allowed to stir for 2 h. The reaction was quenched via dropwise addition of water and the layers were separated. The aqueous layer was extracted with EtOAc (×3). The combined organic layers were dried over MgSO₄, filtered, and concentrated under reduced pressure. The reaction mixture was purified using flash column chromatography (ISCO, 24 g SiO₂, 0-50% Heptane/EtOAc) to afford 7-bromo-5-(trifluoromethyl)-1-{[2 (trimethylsilyl)ethoxy]methyl}-1H-pyrazolo[3,4-c]pyridine (36a) as a brown oil. LCMS (APCI+) 397.0 (M+H)⁺. ¹H NMR (400 MHz, CDCl₃) δ 8.33-8.19 (m, 1H), 8.05 (s, 1H), 6.17 (s, 2H), 3.64 (dd, J=7.6, 8.7 Hz, 2H), 1.00-0.89 (m, 2H), −0.03 (s, 9H).

### Step 2: tert-butyl {2-[5-(trifluoromethyl)-1-{[2-(trimethylsilyl)ethoxy]methyl}-1H-pyrazolo[3,4-c]pyridin-7-yl]prop-2-en-1-yl}carbamate (36b)

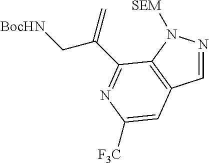 [SMILES: C=C(CNC(=O)OC(C)(C)C)c1nc(C(F)(F)F)cc2cnn(COCC[Si](C)(C)C)c12]

A mixture of 7-bromo-5-(trifluoromethyl)-1-{[2 (trimethylsilyl)ethoxy]methyl}-1H-pyrazolo[3,4-c]pyridine (36a) (210 mg, 0.53 mmol), tert-butyl [2-(4,4,5,5-tetramethyl-1,3,2-dioxaborolan-2-yl)prop-2-en-1-yl]carbamate (150 mg, 0.53 mmol), potassium carbonate (183 mg, 1.32 mmol) and PdCl₂(dppf) (40.8 mg, 0.53 mmol) in dioxane (3.0 mL, 0.1 M) and water (0.6 mL, 0.1 M) was heated at 80° C. for 2 h. The mixture was cooled to room temperature, filtered through celite and washed with DCM, then diluted with water (5 mL) and extracted with DCM (2×5 mL). The combined organic layers were dried over Na₂SO₄ and concentrated under reduced pressure. The residue was purified by flash column chromatography (ISCO, 0-50% EtOAc/heptane) to afford tert-butyl {2-[5-(trifluoromethyl)-1-{[2-(trimethylsilyl)ethoxy]methyl}-1H-pyrazolo[3,4-c]pyridin-7-yl]prop-2-en-1-yl}carbamate (36b) as a pale brown foam (192 mg, 77%). LCMS (APCI+) 473.2 (M+H)⁺. ¹H NMR (400 MHz, CDCl₃) δ 8.23 (s, 1H), 8.01 (s, 1H), 5.94 (s, 2H), 5.85 (s, 1H), 5.54 (s, 1H), 4.33 (d, J=5.9 Hz, 2H), 3.59-3.43 (m, 2H), 1.40 (s, 9H), 0.94-0.87 (m, 2H), −0.02 (s, 9H).

### Step 3: tert-butyl {2-[5-(trifluoromethyl)-1-{[2-(trimethylsilyl)ethoxy]methyl}-1H-pyrazolo[3,4-c]pyridin-7-yl]propyl}carbamate (36c)

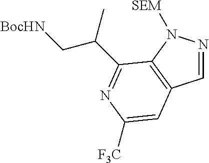 [SMILES: CC(CNC(=O)OC(C)(C)C)c1nc(C(F)(F)F)cc2cnn(COCC[Si](C)(C)C)c12]

tert-butyl {2-[5-(trifluoromethyl)-1-{[2-(trimethylsilyl)ethoxy]methyl}-1H-pyrazolo[3,4-c]pyridin-7-yl]prop-2-en-1-yl}carbamate (36b) (192 mg, 0.406 mmol) was hydrogenated via H-cube (40 bar, 1 mL/min, Pd/C H-Cube, MeOH as solvent). The volatiles were removed under reduced pressure. The crude was purified by flash column chromatography (ISCO, 0-100% EtOAc/heptane) to give tert-butyl {2-[5-(trifluoromethyl)-1-{[2-(trimethylsilyl)ethoxy]methyl}-1H-pyrazolo[3,4-c]pyridin-7-yl]propyl}carbamate (36c) as a colorless oil (167.0 mg, 87%). LCMS (APCI+) 475.2 (M+H)⁺. ¹H NMR (400 MHz, CDCl₃) δ 8.18 (s, 1H), 8.00-7.89 (m, 1H), 6.10 (br d, J=11.5 Hz, 1H), 5.95-5.86 (m, 1H), 5.15 (br s, 1H), 4.12-3.99 (m, 1H), 3.76 (td, J=6.6, 13.6 Hz, 1H), 3.65-3.47 (m, 3H), 1.48-1.41 (m, 12H), 0.96-0.91 (m, 2H), 0.00 (s, 9H).

### Step 4: tert-butyl {2-[5-(trifluoromethyl)-1H-pyrazolo[3,4-c]pyridin-7-yl]propyl}carbamate (36d)

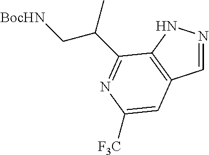 [SMILES: CC(CNC(=O)OC(C)(C)C)c1nc(C(F)(F)F)cc2cn[nH]c12]

To solution of tert-butyl {2-[5-(trifluoromethyl)-1-{[2-(trimethylsilyl)ethoxy]methyl}-1H-pyrazolo[3,4-c]pyridin-7-yl]propyl}carbamate (36c) (165 mg, 0.348 mmol) in THF (1.74 mL, 0.2 M) was added tetrabutylammonium fluoride solution (1.74 mL, 1.74 mmol, 1.0 M). The reaction was heated to 70° C. overnight. The reaction mixture was then cooled and transferred to a separatory funnel and diluted with EtOAc and saturated aqueous NaHCO₃. The layers were separated and the aqueous layer was extracted with EtOAc (2×). The combined organic layers were dried over MgSO₄, filtered, and concentrated under reduced pressure. The residue was purified using flash column chromatography (ISCO, 0-100% EtOAc/heptane) to give tert-butyl {2-[5-(trifluoromethyl)-1H-pyrazolo[3,4-c]pyridin-7-yl]propyl}carbamate (36d) as a white solid (67 mg, 56%). LCMS (APCI+) 345 (M+H)⁺. ¹H NMR (400 MHz, CDCl₃) δ 8.31-8.19 (m, 1H), 7.97 (s, 1H), 5.29-5.16 (m, 1H), 3.84-3.63 (m, 2H), 3.10-2.83 (m, 1H), 1.57 (d, J=6.7 Hz, 3H), 1.53 (s, 9H).

### Step 5: tert-butyl {2-[3-bromo-5-(trifluoromethyl)-1H-pyrazolo[3,4-c]pyridin-7-yl]propyl}carbamate (Intermediate 36) 00110655-7802

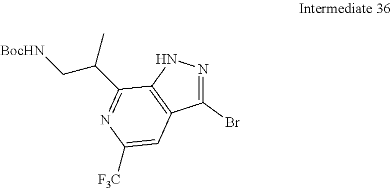 [SMILES: CC(CNC(=O)OC(C)(C)C)c1nc(C(F)(F)F)cc2c(Br)n[nH]c12]

To tert-butyl {2-[5-(trifluoromethyl)-1H-pyrazolo[3,4-c]pyridin-7-yl]propyl}carbamate (36d) (67 mg, 0.19 mmol) in 1,1,1,3,3,3-hexafluoropropan-2-ol (1.95 mL, 0.1 M) was added N-bromosuccinimide (41.6 mg, 0.233 mmol). The mixture was stirred at 40° C. overnight and then quenched with 10% sodium thiosulfate (5 mL) and extracted with DCM (2×5 mL). The combined organic layers were dried over Na₂SO₄, filtered, and concentrated under reduced pressure. The crude material was purified via flash column chromatography (ISCO, 0-100% EtOAc/heptane) to give tert-butyl {2-[3-bromo-5-(trifluoromethyl)-1H-pyrazolo[3,4-c]pyridin-7-yl]propyl}carbamate (Intermediate 36) as a white solid (77 mg, 94%). LCMS (APCI+) 324.9 (M+H-Boc)⁺. ¹H NMR (400 MHz, CDCl₃) δ 7.86 (s, 1H), 5.31 (br s, 1H), 3.82-3.72 (m, 1H), 3.71-3.63 (m, 1H), 3.06-2.94 (m, 1H), 1.56 (d, J=6.7 Hz, 3H), 1.52 (s, 9H).

### EXAMPLES

In order that this disclosure may be better understood, the following examples are set forth. These examples are for purposes of illustration only and are not to be construed as limiting the scope of the disclosure in any manner.

### Preparation of Examples

### Example A1: 3-{3-[(R)-cyclobutyl(4-methyl-4H-1,2,4-triazol-3-yl)methyl]phenyl}-5-(trifluoromethyl)-1H-pyrazolo[3,4-c]pyridine

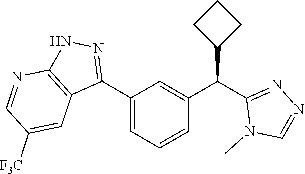 [SMILES: Cn1cnnc1[C@@H](c1cccc(-c2n[nH]c3ncc(C(F)(F)F)cc23)c1)C1CCC1]

Example A1 was prepared according to General Method A.

To a solution of 3-iodo-5-(trifluoromethyl)-1H-pyrazolo[3,4-c]pyridine (54.0 mg, 0.17 mmol) and 3-{(R)-cyclobutyl[3-(4,4,5,5-tetramethyl-1,3,2-dioxaborolan-2-yl)phenyl]methyl}-4-methyl-4H-1,2,4-triazole (Intermediate 1′) (61 mg, 0.15 mmol) in dioxane (4 mL) and H₂O (0.8 mL) was added CsF (78.7 mg, 0.52 mmol) and Pd(dppf)Cl₂. DCM (28.2 mg, 0.03 mmol). The mixture was degassed with N₂ three times. The resulting mixture was heated at 100° C. for 18 hr. The mixture was cooled to RT and added more Pd(dppf)Cl₂·DCM (25.0 mg, 0.03 mmol). The mixture was heated at 100° C. for 20 hr. The mixture was diluted with brine (5 mL) and extracted with DCM (2×5 mL). The combined organic layers were dried over Na₂SO₄, filtered and concentrated. The crude product was purified by flash column chromatography (ISCO, 4 g SiO₂, solvent 0-10% MeOH/1:1 EtOAc/DCM to give the title compound as brown color gum (34 mg). The product was further purified with reverse phase HPLC (Column: ZymorSpher HADP 150×21.2 mm, 5 um; Temperature: 40° C.; Pressure: 120.0 bar; Flow Rate: 95.000 mL/min; Mobile Phase Composition: CO₂; Solvent: MeOH) to give 3-{3-[(R)-cyclobutyl(4-methyl-4H-1,2,4-triazol-3-yl)methyl]phenyl}-5-(trifluoromethyl)-1H-pyrazolo[3,4-c]pyridine (Example A1) as an off-white solid (9.3 mg, 13%). ¹H NMR (400 MHz, DMSO-d₆) δ 14.49-14.04 (m, 1H), 9.25 (s, 1H), 8.40 (s, 1H), 8.34 (s, 1H), 7.98-7.88 (m, 2H), 7.51 (t, J=7.9 Hz, 1H), 7.35 (d, J=7.6 Hz, 1H), 4.38 (d, J=10.5 Hz, 1H), 3.46 (s, 3H), 3.26-3.12 (m, 1H), 2.21-2.04 (m, 1H), 1.93-1.66 (m, 5H); m/z (APCI+) for (C₂₁H₁₉F₃N₆), 413.1 (M+H)⁺. [α]_{D}²² −104.9° (c 0.1, MeOH).

Examples A2-A7 reported in Table 1 were synthesized with non-critical changes or substitutions to the exemplified procedures for Examples A1 that one skilled in the art would be able to realize.

TABLE 1

| | | LCMS | |
|---|---|---|---|
| Example | | (m/z) | |
| Number | Structure and Name | (M + H)⁺ | ¹H NMR and Rotation |
| A2 | 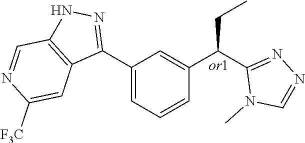 [SMILES: CC[C@H](c1cccc(-c2n[nH]c3cnc(C(F)(F)F)cc23)c1)c1nncn1C] | 387.3 | ¹H NMR (400 MHz, DMSO-d₆) δ 14.30 (br s, 1H), 9.25 (s, 1H), 8.41 (s, 1H), 8.36 (s, 1H), 8.01 − 7.86 (m, 2H), 7.52 (t, J = 7.6 Hz, 1H), 7.36 (d, J = 7.8 Hz, 1H), 4.27 (t, J = 7.5 Hz, 1H), 3.45 (s, 3H), 2.31 − 2.20 (m, 1H), 2.10 − 1.97 (m, 1H), 0.92 (t, J = 7.3 Hz, 3H). |
| | 3-{3-[(1ξ)-1-(4-methyl-4H-1,2,4- | | |
| | triazol-3-yl)propyl]phenyl}-5- | | |
| | (trifluoromethyl)-1H- | | |
| | pyrazolo[3,4-c]pyridine | | |
| A3 | 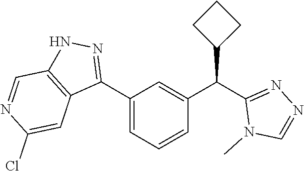 [SMILES: Cn1cnnc1[C@@H](c1cccc(-c2n[nH]c3cnc(Cl)cc23)c1)C1CCC1] | 379.1 | ¹H NMR (400 MHz, 80 ° C., DMSO-d₆) δ 8.86 (d, J = 1.1 Hz, 1H), 8.26 (s, 1H), 7.92 (s, 1H), 7.89 (t, J = 1.6 Hz, 1H), 7.87 − 7.84 (m, 1H), 7.45 (t, J = 7.7 Hz, 1H), 7.27 (d, J = 7.7 Hz, 1H), 4.32 (d, J = 10.2 Hz, 1H), 3.46 (s, 3H), 3.30 − 3.20 (m, 1H), 2.18 − 2.08 (m, 1H), 1.91 − 1.73 (m, 5H). |
| | 5-chloro-3-{3-[(R)-cyclobutyl(4- | | |
| | methyl-4H-1,2,4-triazol-3- | | |
| | yl)methyl]phenyl}-1H- | | |
| | pyrazolo[3,4-c]pyridine | | |
| A4 | 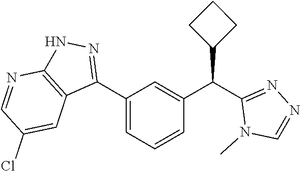 [SMILES: Cn1cnnc1[C@@H](c1cccc(-c2n[nH]c3ncc(Cl)cc23)c1)C1CCC1] | 379.1 | ¹H NMR (600 MHz, DMSO-d₆) δ 8.59 (d, J = 5.4 Hz, 2H), 8.33 (s, 1H), 7.94 − 7.86 (m, 2H), 7.45 (t, J = 1.0 Hz, 1H), 7.28 (d, J = 7.7 Hz, 1H), 4.35 (d, J = 10.5 Hz, 1H), 3.44 (s, 3H), 3.23 − 3.19 (m, 1H), 2.15 − 2.07 (m, 1H), 1.89 − 1.74 (m, 4H), 1.74 − 1.69 (m, 1H). |
| | 5-chloro-3-{3-[(R)-cyclobutyl(4- | | |
| | methyl-4H-1,2,4-triazol-3- | | |
| | yl)methyl]phenyl}-1H- | | |
| | pyrazolo[3,4-b]pyridine | | |
| A5 | 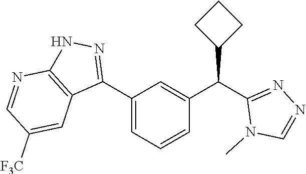 [SMILES: Cn1cnnc1[C@@H](c1cccc(-c2n[nH]c3ncc(C(F)(F)F)cc23)c1)C1CCC1] | 413.1 | ¹H NMR (600 MHz, DMSO-d₆) δ 14.31 (s, 1H), 8.93 (s, 1H), 8.81 (s, 1H), 8.33 (s, 1H), 7.94 (s, 2H), 7.49 (s, 1H), 7.33 (br d, J = 7.6 Hz, 1H), 4.36 (d, J = 10.4 Hz, 1H), 3.45 (s, 3H), 3.24 − 3.18 (m, 1H), 2.15 − 2.05 (m, 1H), 1.89 − 1.76 (m, 4H), 1.76 − 1.68 (m, 1H). |
| | 3-{3-[(R)-cyclobutyl(4-methyl- | | |
| | 4H-1,2,4-triazol-3- | | |
| | yl)methyl]phenyl}-5- | | |
| | (trifluoromethyl)-1H- | | |
| | pyrazolo[3,4-b]pyridine | | |
| A6 | 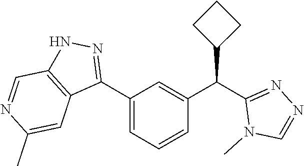 [SMILES: Cc1cc2c(-c3cccc([C@H](c4nncn4C)C4CCC4)c3)n[nH]c2cn1] | 359.3 | ¹H NMR (400 MHz, DMSO-d₆) δ 13.67 (br s, 1H), 8.97 (s, 1H), 8.36 (s, 1H), 7.91 − 7.85 (m, 2H), 7.78 (s, 1H), 7.47 (t, J = 8.0 Hz, 1H), 7.29 (d, J = 7.8 Hz, 1H), 4.34 (d, J = 10.5 Hz, 1H), 3.44 (s, 3H), 3.28 − 3.14 (m, 1H), 2.61 (s, 3H), 2.17 − 2.06 (m, 1H), 1.90 − 1.66 (m, 5H). |
| | 3-{3-[(R)-cyclobutyl(4-methyl- | | |
| | 4H-1,2,4-triazol-3- | | |
| | yl)methyl]phenyl}-5-methyl-1H- | | |
| | pyrazolo[3,4-c]pyridine | | |
| A7 | 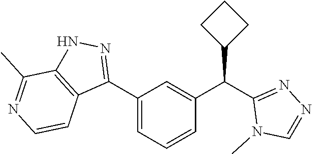 [SMILES: Cc1nccc2c(-c3cccc([C@H](c4nncn4C)C4CCC4)c3)n[nH]c12] | 359.3 | ¹H NMR (400 MHz, DMSO-d₆) δ 13.87 (s, 1H), 8.34 (s, 1H), 8.16 (d, J = 5.8 Hz, 1H), 7.89 (s, 1H), 7.87 (br d, J = 7.8 Hz, 1H), 7.78 (d, J = 5.6 Hz, 1H), 7.46 (t, J = 7.7 Hz, 1H), 7.28 (br d, J = 7.5 Hz, 1H), 4.34 (d, J = 10.5 Hz, 1H), 3.44 (s, 3H), 3.27 − 3.15 (m, 1H), 2.77 (s, 3H), 2.15 − 2.05 (m, 1H), 1.90 − 1.70 (m, 6H). |
| | 3-{3-[(R)-cyclobutyl(4-methyl- | | |
| | 4H-1,2,4-triazol-3- | | |
| | yl)methyl]phenyl}-7-methyl-1H- | | |
| | pyrazolo[3,4-c]pyridine | | |

### Example B1: 3-(3-(3-((4-methyl-4H-1,2,4-triazol-3-yl)methyl)oxetan-3-yl)phenyl)-5-(trifluoromethyl)-1H-pyrazolo[3,4-c]pyridine

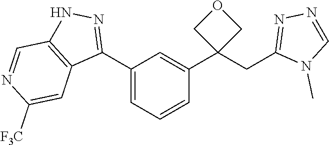 [SMILES: Cn1cnnc1CC1(c2cccc(-c3n[nH]c4cnc(C(F)(F)F)cc34)c2)COC1]

Example B1 was prepared according to General Method B.

A mixture of 4,4,4′,4′,5,5,5′,5′-octamethyl-2,2′-bi(1,3,2-dioxaborolane (62 mg, 0.243 mmol, 1.5 equiv), 3-((3-(3-bromophenyl)oxetan-3-yl)methyl)-4-methyl-4H-1,2,4-triazole (2e) (50.0 mg, 0.16 mmol, 1 equiv) and potassium acetate (47.8 mg, 0.487 mmol, 3 equiv) in 1,4-dioxane (1 mL) was deoxygenated with a N₂ bubbler for a few minutes before the addition of 1,1′-bis(diphenylphosphino)ferrocene-Palladium(II)dichloride dichloromethane complex (13 mg, 0.0162 mmol, 10 mol %). The mixture was capped and heated at 120° C. in the microwave for 40 min. The mixture was uncapped and to this mixture was added 3-bromo-5-(trifluoromethyl)-1H-pyrazolo[3,4-c]pyridine (65 mg, 0.243 mmol, 1.5 equiv). The mixture was diluted with dioxane (0.5 mL) and ethanol (0.5 mL) then treated with 1M aqueous cesium carbonate solution (0.25 mL). The mixture was deoxygenated with a N₂ bubbler for a few minutes before the addition of tetrakis triphenylphosphine palladium (19 mg, 0.0162 mmol, 10 mol %). The mixture was sealed and heated in the microwave at 140° C. for 2 h.

The mixture was uncapped and deoxygenated with N₂ for a few minutes then more 3-bromo-5-(trifluoromethyl)-1H-pyrazolo[3,4-c]pyridine (30 mg, 0.114 mmol, 0.7 equiv), 1M aq cesium carbonate solution (0.250 mL, 1.0 M) and tetrakis triphenylphosphine palladium (10 mg, 0.0087 mmol, 5 mol %) were added. The mixture was sealed and heated in the microwave at 140° C. for 1 h. The mixture was uncapped and deoxygenated with N₂ for a few minutes and more tetrakis triphenylphosphine palladium (10 mg, 0.0087 mmol, 5 mol %) was added. The mixture was sealed and heated in the microwave at 140° C. for 1 h. The mixture was then partitioned between brine/EtOAc. The aqueous layer was extracted with EtOAc (2×). The combined organics were washed with brine, dried over MgSO₄, filtered, and concentrated under reduced pressure. The residue was taken up in MeOH and filtered through a 0.45 micron syringe filter. The filtrate was purified by SFC (Agilent SFC/HPLC Hybrid Princeton SFC HA Morpholine 5 μm 4.6×150 mm column, 10-50% MeOH in CO₂ over 3.4 mins; 160 bar; 4.0 mL/min; Peak @ 1.93 min) to give 3-(3-(3-((4-methyl-4H-1,2,4-triazol-3-yl)methyl)oxetan-3-yl)phenyl)-5-(trifluoromethyl)-1H-pyrazolo[3,4-c]pyridine (Example B1) (6.9 mg, 10%). LCMS (APCI+) 415.1 (M+H). ¹H NMR (700 MHz, DMSO-d₆) δ 14.27 (br. s, 1H), 9.24 (s, 1H), 8.37 (s, 1H), 8.18 (s, 1H), 7.93-7.90 (m, 1H), 7.62 (t, J=1.5 Hz, 1H), 7.48 (t, J=7.7 Hz, 1H), 7.09-7.07 (m, 1H), 4.99 (d, J=6.0 Hz, 2H), 4.94 (d, J=6.0 Hz, 2H), 3.58 (s, 2H), 2.98 (s, 3H).

Examples B2-B10 reported in Table 2 were synthesized with non-critical changes or substitutions to the exemplified procedures for Example B1 that one skilled in the art would be able to realize.

TABLE 2

| | | LCMS | |
|---|---|---|---|
| Example | | (m/z) | |
| Number | Structure and Name | (M + H)⁺ | ¹H NMR and Rotation |
| B2 | 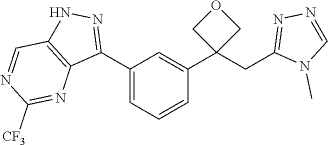 [SMILES: Cn1cnnc1CC1(c2cccc(-c3n[nH]c4cnc(C(F)(F)F)nc34)c2)COC1] | 416.1 | ¹H NMR (700 MHz, DMSO-d₆) δ 9.51 − 9.38 (m, 1H), 8.32 (d, J = 7.7 Hz, 1H), 8.12 (s, 1H), 8.08 (s, 1H), 7.46 (t, J = 7.7 Hz, 1H), 6.98 (br. d, J = 8.0 Hz, 1H), 5.02 (d, J = 6.0 Hz, 2H), 4.96 (d, J = 5.8 Hz, 2H), 3.56 (s, 2H), 3.00 (s, 3H) |
| | 3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[4,3-d]pyrimidine | | |
| B3 | 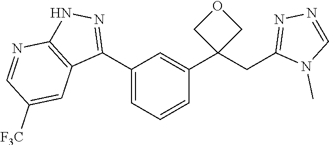 [SMILES: Cn1cnnc1CC1(c2cccc(-c3n[nH]c4ncc(C(F)(F)F)cc34)c2)COC1] | 415.2 | ¹H NMR (400MHz, DMSO-d₆) δ 14.30 (br s, 1H), 8.93 (d, J = 1.7 Hz, 1H), 8.78 (d, J = 1.2 Hz, 1H), 8.18 (s, 1H), 7.93 (d, J = 7.9 Hz, 1H), 7.62 (t, J = 1.6 Hz, 1H), 7.47 (t, J = 7.8 Hz, 1H), 7.08 (d, J = 8.4 Hz, 1H), 5.02 − 4.97 (m, 2H), 4.97 − 4.92 (m, 2H), 3.58 (s, 2H), 2.97 (s, 3H). |
| | 3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-b]pyridine | | |
| B4 | 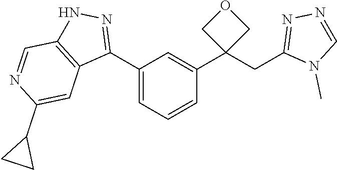 [SMILES: Cn1cnnc1CC1(c2cccc(-c3n[nH]c4cnc(C5CC5)cc34)c2)COC1] | 387.3 | ¹H NMR (400 MHz, CDCl₃) δ 9.00 (br s, 1H), 7.92 − 7.81 (m, 2H), 7.58 (s, 1H), 7.45 (t, J = 7.7 Hz, 1H), 7.41 (br s, 1H), 6.87 (br d, J = 8.1 Hz, 1H), 5.22 − 5.17 (m, 2H), 5.17 − 5.12 (m, 2H), 3.63 (s, 2H), 2.79 (s, 3H), 2.31 − 2.24 (m, 1H), 1.10 − 1.03 (m, 4H). |
| | 5-cyclopropyl-3-(3-{3-[(4- | | |
| | methyl-4H-1,2,4-triazol-3- | | |
| | yl)methyl]oxetan-3-yl}phenyl)- | | |
| | 1H-pyrazolo[3,4-c]pyridine | | |
| B5 | 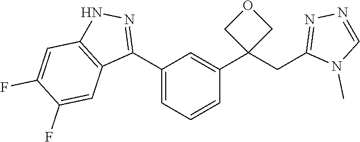 [SMILES: Cn1cnnc1CC1(c2cccc(-c3n[nH]c4cc(F)c(F)cc34)c2)COC1] | 382 | |
| | 5,6-difluoro-3-(3-{3-[(4- | | |
| | methyl-4H-1,2,4-triazol-3- | | |
| | yl)methyl]oxetan-3-yl}phenyl)- | | |
| | 1H-indazole | | |
| B6 | 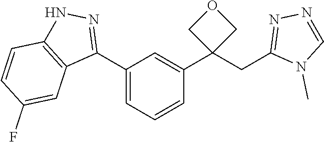 [SMILES: Cn1cnnc1CC1(c2cccc(-c3n[nH]c4ccc(F)cc34)c2)COC1] | 364 | |
| | 5-fluoro-3-(3-{3-[(4-methyl- | | |
| | 4H-1,2,4-triazol-3- | | |
| | yl)methyl]oxetan-3-yl}phenyl)- | | |
| | 1H-indazole | | |
| B7 | 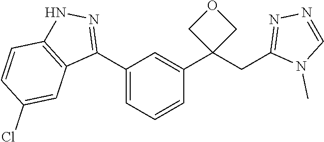 [SMILES: Cn1cnnc1CC1(c2cccc(-c3n[nH]c4ccc(Cl)cc34)c2)COC1] | 380 | |
| | 5-chloro-3-(3-{3-[(4-methyl- | | |
| | 4H-1,2,4-triazol-3- | | |
| | yl)methyl]oxetan-3-yl}phenyl)- | | |
| | 1H-indazole | | |
| B8 | 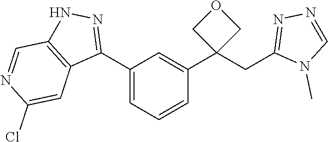 [SMILES: Cn1cnnc1CC1(c2cccc(-c3n[nH]c4cnc(Cl)cc34)c2)COC1] | 381.1 | ¹H NMR (400 MHz, DMSO-d₆) δ 13.98 (br. s, 1H), 8.93 (s, 1H), 8.19 (s, 1H), 8.00 (d, J = 1.2 Hz, 1H), 7.87 (dt, J = 7.8, 1.3 Hz, 1H), 7.56 (t, J = 1.8 Hz, 1H), 7.45 (t, J = 7.7 Hz, 1H), 7.06 − 7.02 (m, 1H), 5.00 (d, J = 6.1 Hz, 2H), 4.94 (d, J = 6.1 Hz, 2H), 3.58 (s, 2H), 2.97 (s, 3H). |
| | 5-chloro-3-(3-{3-[(4-methyl- | | |
| | 4H-1,2,4-triazol-3- | | |
| | yl)methyl]oxetan-3-yl}phenyl)- | | |
| | 1H-pyrazolo[3,4-c]pyridine | | |
| B9 | 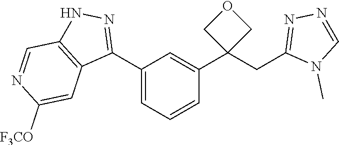 [SMILES: Cn1cnnc1CC1(c2cccc(-c3n[nH]c4cnc(OC(F)(F)F)cc34)c2)COC1] | 430.1 | ¹H NMR (600 MHz, DMSO-d₆) δ 8.85 (s, 1H), 8.17 (s, 1H), 7.86 (d, J = 7.8 Hz, 1H), 7.69 (s, 1H), 7.57 (s, 1H), 7.40 (t, J = 7.7 Hz, 1H), 6.90 (d, J = 7.6 Hz, 1H), 5.05 − 4.90 (m, 4H), 3.57 (s, 2H), 2.92 (s, 3H). |
| | 3-(3-{3-[(4-methyl-4H-1,2,4- | | |
| | triazol-3-yl)methyl]oxetan-3- | | |
| | yl}phenyl)-5- | | |
| | (trifluoromethoxy)-1H- | | |
| | pyrazolo[3,4-c]pyridine | | |
| B10 | 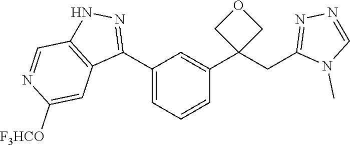 | 412.1 | ¹H NMR (600 MHz, DMSO-d₆) δ 8.83 (s, 1H), 8.17 (s, 1H), 7.87 (d, J = 7.7 Hz, 1H), 7.82 − 7.54 (m, 3H), 7.44 (t, J = 7.7 Hz, 1H), 7.00 (d, J = 7.9 Hz, 1H), 5.03 − 4.91 (m, 4H), 3.58 (s, 2H), 2.95 (s, 3H). |
| | 5-(difluoromethoxy)-3-(3-{3- | | |
| | [(4-methyl-4H-1,2,4-triazol-3- | | |
| | yl)methyl]oxetan-3-yl}phenyl)- | | |
| | 1H-pyrazolo[3,4-c]pyridine | | |

### Example C1: (1S,2R)-2-({[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}amino)cyclopentan-1-ol

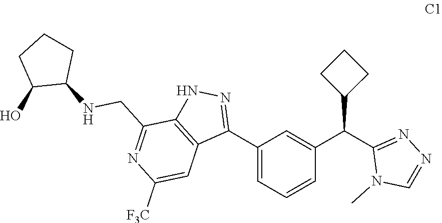 [SMILES: Cn1cnnc1[C@@H](c1cccc(-c2n[nH]c3c(CN[C@@H]4CCC[C@@H]4O)nc(C(F)(F)F)cc23)c1)C1CCC1]

Example C1 was prepared according to General Method C.

To a mixture of (R)-3-(3-(cyclobutyl(4-methyl-4H-1,2,4-triazol-3-yl)methyl)phenyl)-5-(trifluoromethyl)-1H-pyrazolo[3,4-c]pyridine-7-carbaldehyde (Intermediate 7) (30.0 mg, 0.0681 mmol) in methanol (0.681 mL) was added (1S,2R)-2-aminocyclopentanol hydrochloride (93.7 mg, 0.681 mmol), triethylamine (34.5 mg, 0.341 mmol, 0.0475 mL) and Ti(OEt)₄ (124 mg, 0.545 mmol, 0.114 mL) and the mixture was stirred at room temperature for 3 h. Then, sodium cyanoborohydride (21.4 mg, 0.341 mmol) was added and the reaction was stirred at room temperature overnight. The reaction was quenched with water and filtered over compacted celite. The filtrate was concentrated, and the crude residue was purified by Prep HPLC (Phenomenex Gemini NX C18 150×21.2 mm, 5 um column Mobile phase A: Water+10 mM Ammonium Acetate Mobile phase B: Acetonitrile 20-50% B in 8.0 minutes, 40 mL/min) to provide (1S,2R)-2-({[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}amino)cyclopentan-1-ol (Example C1) as a white solid (2.7 mg, 7.5%). LCMS (ESI+) 526.05 (M+H). ¹H NMR (600 MHz, DMSO-d₆) δ 8.32 (s, 1H), 8.18 (s, 1H), 7.90-7.85 (m, 2H), 7.46 (t, 1H), 7.27 (d, 1H), 4.33 (d, 1H), 4.30-4.22 (m, 2H), 4.03-3.97 (m, 1H), 3.43 (s, 3H), 2.88-2.86 (m, 1H), 2.10 (td, 1H), 1.94-1.80 (m, 4H), 1.77-1.54 (m, 6H), 1.48-1.33 (m, 2H).

### Example C2: (3S)—N-{(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine; and

### Example C3: (3S)—N-{(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine

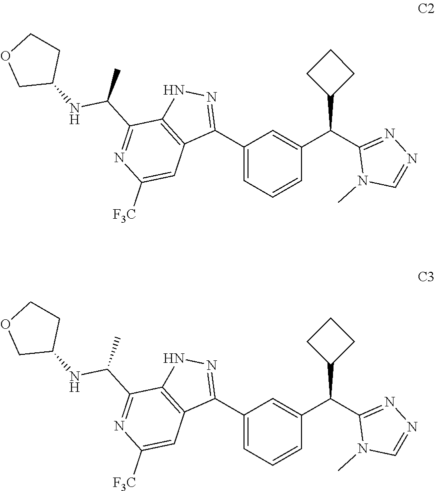 [SMILES: C[C@H](N[C@H]1CCOC1)c1nc(C(F)(F)F)cc2c(-c3cccc([C@H](c4nncn4C)C4CCC4)c3)n[nH]c12 ; C[C@@H](N[C@H]1CCOC1)c1nc(C(F)(F)F)cc2c(-c3cccc([C@H](c4nncn4C)C4CCC4)c3)n[nH]c12]

Examples C2 and C3 were prepared according to General Method C.

To a solution of 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-one (Intermediate 8) (257 mg, 0.566 mmol) in EtOH (3 mL), was added (S)-3-aminotetrahydrofuran hydrochloride (349 mg, 2.83 mmol), Et₃N (458 mg, 4.52 mmol, 0.631 mL), and then Ti(OEt)₄ (774 mg, 3.39 mmol, 0.711 mL). The mixture was heated at 70° C. for 3 h, then cooled to RT, and NaBH₃CN (178 mg, 2.83 mmol) was added. The resulting mixture was stirred at RT for 18 h. The mixture was quenched with saturated aqueous NaHCO₃ (10 mL) and diluted with DCM (10 mL) and stirred at RT for 30 min.

The mixture was then filtered through Celite and washed with DCM. The layers were separated, and the aqueous layer was extracted with DCM (10 mL). The combined organic layers were dried over Na₂SO₄, filtered, and concentrated under reduced pressure. The crude residue was purified by preparative SFC with ES Industries Chromega Chiral CCOF₄ column (250×20 mm, 5 um particle size), Temperature: 35° C., Pressure: 120.0 bar, Flow Rate: 100.000 mL/min, eluted with 10.0% MeOH+10 mM NH₃ in CO₂, Mobile Phase Composition: CO₂; Solvent: MeOH+10 mM NH₃). (3S)—N-{(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine (Example C2) was obtained as the first eluting peak as white solid (29.9 mg, 10%, >99.0% ee). LCMS (ESI+) 526.0 (M+H). ¹H NMR (400 MHz, CD₃OD) δ 8.38 (s, 1H), 8.17 (s, 1H), 7.89 (d, J=7.7 Hz, 1H), 7.85 (s, 1H), 7.55 (t, J=7.7 Hz, 1H), 7.37 (d, J=7.9 Hz, 1H), 4.52 (q, J=6.8 Hz, 1H), 4.34 (d, J=10.9 Hz, 1H), 3.96 (dt, J=6.5, 8.0 Hz, 1H), 3.74 (dt, J=6.2, 8.1 Hz, 1H), 3.66 (dd, J=6.1, 8.8 Hz, 1H), 3.55 (s, 3H), 3.51 (dd, J=4.6, 8.8 Hz, 1H), 3.41-3.35 (m, 1H), 3.31-3.24 (m, 1H), 2.38-2.26 (m, 1H), 2.15-2.04 (m, 1H), 2.04-1.87 (m, 5H), 1.87-1.75 (m, 1H), 1.56 (d, J=6.7 Hz, 3H). [α]_{D}²²=−39.2° (c 0.1, MeOH). (3S)—N-{(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine (Example C3) was obtained as the second eluting peak as white solid (35.8 mg, 12%, ˜93.0% ee, ˜96.0% pure). LCMS (ESI+) 526.0 (M+H). ¹H NMR (400 MHz, MeOH-d₄) δ 8.38 (s, 1H), 8.17 (s, 1H), 7.89 (d, J=7.7 Hz, 1H), 7.85 (s, 1H), 7.55 (t, J=7.7 Hz, 1H), 7.37 (d, J=7.9 Hz, 1H), 4.52 (q, J=6.8 Hz, 1H), 4.34 (d, J=10.9 Hz, 1H), 3.96 (dt, J=6.5, 8.0 Hz, 1H), 3.74 (dt, J=6.2, 8.1 Hz, 1H), 3.66 (dd, J=6.1, 8.8 Hz, 1H), 3.55 (s, 3H), 3.51 (dd, J=4.6, 8.8 Hz, 1H), 3.41-3.35 (m, 1H), 3.31-3.24 (m, 1H), 2.38-2.26 (m, 1H), 2.15-2.04 (m, 1H), 2.04-1.87 (m, 5H), 1.87-1.75 (m, 1H), 1.56 (d, J=6.7 Hz, 3H). [α]_{D}²²=−72.7° (C 0.1, MeOH).

### Example C39: 2-({(1ξ)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethyl}amino)ethanol; and

### Example C40: 2-({(1ξ)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethyl}amino)ethanol

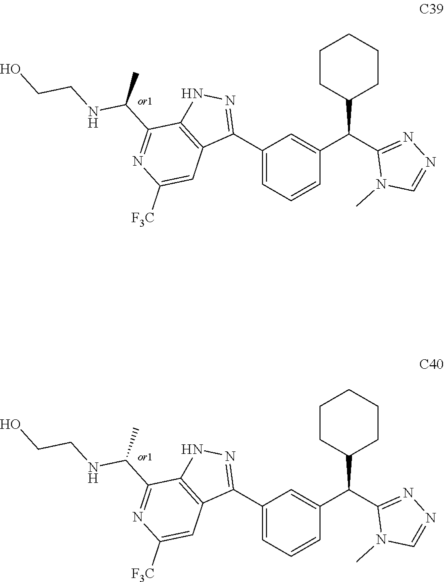 [SMILES: C[C@H](NCCO)c1nc(C(F)(F)F)cc2c(-c3cccc([C@H](c4nncn4C)C4CCCCC4)c3)n[nH]c12 ; C[C@@H](NCCO)c1nc(C(F)(F)F)cc2c(-c3cccc([C@H](c4nncn4C)C4CCCCC4)c3)n[nH]c12]

Examples C39 and C40 were prepared according to General Method C.

To a solution of 1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-one (Intermediate 18) (181 mg, 0.375 mmol) in EtOH (4.7 mL), was added ethanolamine (0.181 mL, 3.00 mmol), and MgSO₄ (226 mg, 1.88 mmol). The mixture was stirred at rt for 24 h, then AcOH (0.0215 mL, 0.375 mmol) and NaBH₄ (85.2 mg, 2.25 mmol) were added. The resulting mixture was stirred at RT for 18 h. The mixture was quenched with water (5 mL) and ammonium acetate solution (0.967 g, 11.3 mmol in 10 mL water). The mixture was stirred at RT for 10 min. The layers were separated, and the aqueous layer was extracted with DCM (10 mL) followed by 10% DCM/i-PrOH (10 mL). The combined organic layers were dried over Na₂SO₄, filtered, and concentrated under reduced pressure. The crude residue was purified by flash column chromatography (Isco, SiO₂, 0-10% MeOH/1:1 DCM/EtOAc to 10% MeOH in DCM to 10% 7N NH₃ in MeOH/DCM) to give the crude residue (85 mg). The crude residue was further purified via preparative SFC with Princeton SFC HA-Morpholine column (150×21.2 mm, 5 um particle size), Temperature: 40° C., Pressure: 120.0 bar, Flow Rate: 100.000 mL/min, eluted with 12-30% MeOH+10 mM NH₃ in CO₂, Mobile Phase Composition: CO₂; Solvent: MeOH+10 mM NH₃). 2-({(1ξ)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethyl}amino)ethanol (Example C39) was obtained as the first eluting peak as white solid (14.7 mg, 7%, >99.0% ee). LCMS (APCI+) 528.2 (M+H)⁺. ¹H NMR (400 MHz, CD₃OD) δ 8.23 (s, 1H), 8.06 (s, 1H), 7.82 (s, 1H), 7.77-7.68 (m, 1H), 7.53-7.39 (m, 1H), 7.33 (d, J=7.8 Hz, 1H), 4.37 (q, J=6.7 Hz, 1H), 3.93 (d, J=10.3 Hz, 1H), 3.65-3.54 (m, 2H), 3.51 (s, 3H), 2.69 (td, J=5.1, 12.2 Hz, 1H), 2.50 (ddd, J=5.0, 6.8, 12.0 Hz, 1H), 2.35 (tq, J=3.0, 10.9 Hz, 1H), 1.78 (br d, J=12.5 Hz, 1H), 1.70-1.56 (m, 3H), 1.46 (d, J=6.7 Hz, 4H), 1.38-1.23 (m, 1H), 1.21-1.11 (m, 2H), 1.04-0.83 (m, 2H); ¹⁹F NMR (376 MHz, CD₃OD) δ −67.5. [α]_{D}²² −15.8° (c 0.1, MeOH). 2-({(1ξ)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethyl}amino)ethanol (Example C40) was obtained as the second eluting peak as white solid (16.1 mg, 8%, ˜93.0% ee). LCMS (APCI+) 528.2 (M+H)⁺. ¹H NMR (400 MHz, CD₃OD) δ 8.23 (s, 1H), 8.05 (s, 1H), 7.82 (s, 1H), 7.75 (d, J=7.7 Hz, 1H), 7.43 (t, J=7.7 Hz, 1H), 7.33 (d, J=7.7 Hz, 1H), 4.36 (q, J=6.7 Hz, 1H), 3.92 (d, J=10.3 Hz, 1H), 3.71-3.55 (m, 2H), 3.51 (s, 3H), 2.68 (td, J=5.1, 12.1 Hz, 1H), 2.49 (ddd, J=5.0, 6.7, 12.0 Hz, 1H), 2.42-2.23 (m, 1H), 1.78 (br d, J=13.1 Hz, 1H), 1.69-1.54 (m, 3H), 1.46 (d, J=6.8 Hz, 3H), 1.37-1.25 (m, 1H), 1.22-1.09 (m, 3H), 1.06-0.87 (m, 2H); ¹⁹F NMR (376 MHz, CD₃OD) δ −67.5. [α]_{D}²² −48.7° (C 0.1, MeOH).

### Example C46: (1S,2S)-2-({(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)cyclopentan-1-ol; and

### Example C47: (1S,2S)-2-({(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)cyclopentan-1-ol

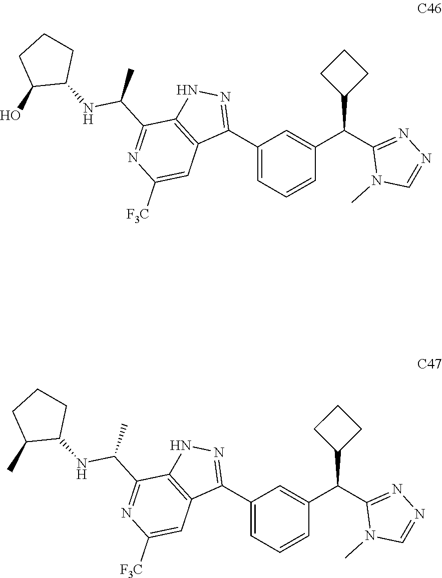

Examples C46 and C47 were prepared according to General Method C.

To a solution of 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-one (Intermediate 8) (152 mg, 0.334 mmol) in EtOH (3 mL), was added (1S,2S)-2-aminocyclopentan-1-ol hydrochloride (138 mg, 1.00 mmol), Et₃N (271 mg, 2.68 mmol, 0.373 mL), and then Ti(OEt)₄ (458 mg, 2.01 mmol, 0.421 mL). The mixture was heated at 50° C. for 4 h, then cooled to RT, and NaBH₄ (63.3 mg, 1.67 mmol) was added. The resulting mixture was stirred at RT for 2 h. The mixture was quenched with saturated aqueous NaHCO₃ (10 mL) and diluted with DCM (10 mL) and stirred at RT for 30 min. The mixture was then filtered through Celite and washed with DCM. To the filtrate was added 7N ammonia in MeOH and stirred at RT for 30 min. The layers were separated, and the aqueous layer was extracted with !0% MeOH/DCM (10 mL). The combined organic layers were dried over Na₂SO₄, filtered, and concentrated under reduced pressure. The crude residue was purified by flash column chromatography (Isco, SiO₂, 0-10% MeOH/DCM to 10% 7 N NH₃ in MeOH/DCM) followed by preparative SFC with Phenomenex Lux Cellulose-2 column (250×21.2 mm, 5 um particle size), Temperature: 35° C., Pressure: 120.0 bar, Flow Rate: 100.000 mL/min, eluted with 25.0% MeOH+10 mM NH₃ in CO₂, Mobile Phase Composition: CO₂; Solvent: MeOH+10 mM NH₃). (1 S,2S)-2-({(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)cyclopentan-1-ol (Example C46) was obtained as the first eluting peak as a white solid (42 mg, 23%, >99.0% de). LCMS 540.3 (M+H)⁺. ¹H NMR (600 MHz, DMSO-d₆) δ 8.33 (s, 1H), 8.21 (s, 1H), 7.94-7.85 (m, 2H), 7.50 (t, J=7.9 Hz, 1H), 7.33 (d, J=7.7 Hz, 1H), 4.44-4.33 (m, 2H), 3.79-3.75 (m, 1H), 3.69-3.66 (m, 1H), 3.44 (s, 3H), 3.23-3.19 (m, 1H), 2.58-2.51 (m, 1H), 2.16-2.06 (m, 1H), 1.89-1.77 (m, 5H), 1.77-1.69 (m, 2H), 1.62-1.47 (m, 2H), 1.44 (d, J=6.8 Hz, 3H), 1.40-1.31 (m, 1H), 1.31-1.21 (m, 1H). [α]_{D}²² −25.0° (c 0.1, MeOH). (1 S,2S)-2-({(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)cyclopentan-1-ol (Example C47) was obtained as the second eluting peak as white solid (37.8 mg, 21%, ˜99.0% de). LCMS 540.3 (M+H)⁺. ¹H NMR (600 MHz, DMSO-d₆) δ 8.27 (s, 1H), 8.14 (s, 1H), 7.87-7.78 (m, 2H), 7.44 (t, J=7.9 Hz, 1H), 7.26 (d, J=7.8 Hz, 1H), 4.42 (q, J=6.8 Hz, 1H), 4.29 (d, J=10.4 Hz, 1H), 3.80-3.77 (m, 1H), 3.73-3.69 (m, 1H), 3.38 (s, 3H), 3.17-3.12 (m, 1H), 2.66-2.59 (m, 1H), 2.10-2.00 (m, 1H), 1.81-1.71 (m, 5H), 1.70-1.62 (m, 1H), 1.57-1.49 (m, 1H), 1.45-1.38 (m, 2H), 1.35 (d, J=6.8 Hz, 3H), 1.32-1.22 (in, 1H), 1.08-1.00 (in, 1H). [α]_{D}²² −44.2° (c 0.1, MeOH).

Examples C4-C38, C41-C45, and C48-C125 reported in Table 3 were synthesized with non-critical changes or substitutions to the exemplified procedures for Examples C1, C2, C3, C39 and C40 that one skilled in the art would be able to realize.

TABLE 3

| | | LCMS | |
|---|---|---|---|
| Example | | (m/z) | |
| Number | Structure and Name | (M + H)⁺ | ¹H NMR and Rotation |
| C4 | 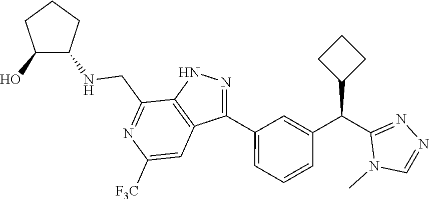 [SMILES: Cn1cnnc1[C@@H](c1cccc(-c2n[nH]c3c(CN[C@H]4CCC[C@@H]4O)nc(C(F)(F)F)cc23)c1)C1CCC1] | 526.1 | ¹H NMR (400 MHz, CD₃OD) δ 8.26 (s, 1H), 8.07 (s, 1H), 7.78 (d, J = 7.4 Hz, 1H), 7.74 (s, 1H), 7.44 (t, J = 7.70 Hz, 1H), 7.25 (d, J = 7.8 Hz, 1H), 4.39-4.26 (m, 2H), 4.26-4.19 (m, 1H), 3.92 (q, J = 6.19 Hz, 1H), 3.43 (s, 3H), 2.95-2.89 (m, 1H), 2.23-2.10 (m, 1H), 2.06-1.83 (m, 7H), 1.74-1.60 (m, 3H), 1.52-1.32 (m, 2H). |
| | (1S,2S)-2-({[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}amino)cyclopentan-1- | | |
| | ol | | |
| C5 | 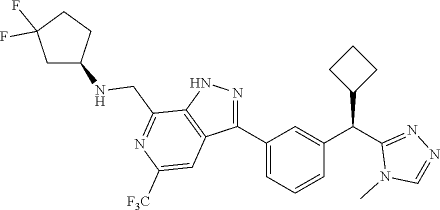 [SMILES: Cn1cnnc1[C@@H](c1cccc(-c2n[nH]c3c(CN[C@@H]4CCC(F)(F)C4)nc(C(F)(F)F)cc23)c1)C1CCC1] | 546.0 | ¹H NMR (600 MHz, DMSO-d₆) δ 8.33 (s, 1H), 8.23 (s, 1H), 7.94- 7.84 (m, 2H), 7.48 (t, J = 7.6 Hz, 1H), 7.31 (d, J = 7.7 Hz, 1H), 4.35 (d, J = 10.5 Hz, 1H), 4.23 (s, 2H), 3.44 (s, 3H), 3.27 (td, J = 14.0, 7.0 Hz, 1H), 3.23-3.18 (m, 1H), 2.38-2.28 (m, 1H), 2.25-2.14 (m, 1H), 2.13-2.06 (m, 1H), 2.02-1.91 (m, 3H), 1.86-1.76 (m, 4H), 1.75-1.69 (m, 1H), 1.67-1.57 (m, 1H). [a]_{D}²² = −55.4° (c 0.1, MeOH) |
| | (1R)-N-{[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]cyclopen-7- | | |
| | yl]methyl}-3,3- | | |
| | difluorocyclopentan-1-amine | | |
| C6 | 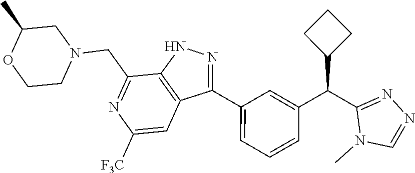 [SMILES: C[C@H]1CN(Cc2nc(C(F)(F)F)cc3c(-c4cccc([C@H](c5nncn5C)C5CCC5)c4)n[nH]c23)CCO1] | 526.0 | ¹H NMR (600 MHz, DMSO-d₆) δ 8.32 (s, 1H), 8.25 (s, 1H), 7.95- 7.83 (m, 2H), 7.53-7.44 (m, 1H), 7.31 (d, J = 7.7 Hz, 1H), 4.35 (d, J = 10.5 Hz, 1H), 3.99 (s, 2H), 3.71-3.66 (m, 1H), 3.62- 3.55 (m, 2H), 3.44 (s, 3H), 3.21- 3.19 (m, 1H), 2.75 (d, J = 11.0 Hz, 1H), 2.67 (d, J = 11.0 Hz, 1H), 2.19 (dt, J = 11.3, 3.3 Hz, 1H), 2.10 (dd, J = 7.2, 3.6 Hz, |
| | 3-{3-[(R)-cyclobutyl(4-methyl- | | 1H), 1.94-1.84 (m, 2H), 1.83- |
| | 4H-1,2,4-triazol-3- | | 1.65 (m, 4H), 0.99 (d, J = 6.4 Hz, |
| | yl)methyl]phenyl}-7-{[(2S)-2- | | 3H). |
| | methylmorpholin-4-yl]methyl}-5- | | |
| | (trifluoromethyl)-1H- | | |
| | pyrazolo[3,4-c]pyridine | | |
| C7 | 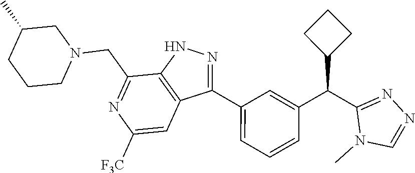 [SMILES: C[C@H]1CCCN(Cc2nc(C(F)(F)F)cc3c(-c4cccc([C@H](c5nncn5C)C5CCC5)c4)n[nH]c23)C1] | 524.0 | ¹H NMR (600 MHz, DMSO-d₆) δ 8.33 (s, 1H), 8.25 (s, 1H), 7.95- 7.83 (m, 2H), 7.49 (t, J = 8.0 Hz, 1H), 7.32 (d, J = 7.7 Hz, 1H), 4.35 (d, J = 10.5 Hz, 1H), 4.03- 3.90 (m, 2H), 3.44 (s, 3H), 3.23- 3.19 (m, 1H), 2.78 (d, J = 7.9 Hz, 2H), 2.14-2.07 (m, 1H), 2.03- 1.94 (m, 1H), 1.90-1.76 (m, 4H), 1.74-1.66 (m, 3H), 1.63 (d, J = 13.2 Hz, 1H), 1.58-1.50 (m, |
| | 3-{3-[(R)-cyclobutyl(4-methyl- | | 2H), 0.87-0.79 (m, 1H), 0.77 (d, |
| | 4H-1,2,4-triazol-3- | | J = 5.9 Hz, 3H). |
| | yl)methyl]phenyl}-7-{[(3S)-3- | | |
| | methylpiperidin-1-yl]methyl}-5- | | |
| | (trifluoromethyl)-1H- | | |
| | pyrazolo[3,4-c]pyridine | | |
| C8 | 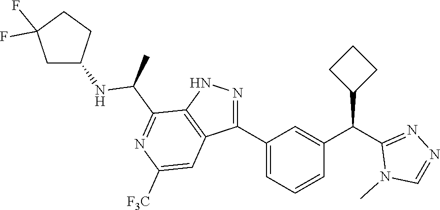 [SMILES: C[C@H](N[C@H]1CCC(F)(F)C1)c1nc(C(F)(F)F)cc2c(-c3cccc([C@H](c4nncn4C)C4CCC4)c3)n[nH]c12] | 560.4 | ¹H NMR (400 MHz, CD₃OD) δ 8.37 (s, 1H), 8.15 (s, 1H), 7.88 (d, J = 7.8 Hz, 1H), 7.84 (s, 1H), 7.54 (t, J = 7.7 Hz, 1H), 7.35 (d, J = 7.7 Hz, 1H), 4.45 (q, J = 6.8 Hz, 1H), 4.33 (d, J = 10.9 Hz, 1H), 3.53 (s, 3H), 3.37-3.32 (m, 1H), 3.17-3.06 (m, 1H), 2.47- 2.23 (m, 2H), 2.20-1.71 (m, 10H), 1.52 (d, J = 6.8 Hz, 3H) |
| | (1S)-N-{(1S)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | ylethyl}-3,3- | | |
| | difluorocyclopentan-1-amine | | |
| C9 | 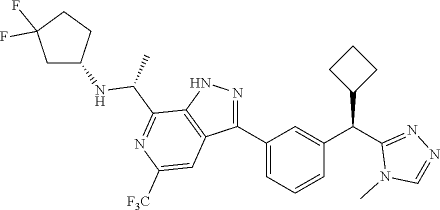 [SMILES: C[C@@H](N[C@H]1CCC(F)(F)C1)c1nc(C(F)(F)F)cc2c(-c3cccc([C@H](c4nncn4C)C4CCC4)c3)n[nH]c12] | 560.4 | ¹H NMR (400 MHz, CD₃OD) δ 8.37 (s, 1H), 8.16 (s, 1H), 7.88 (d, J = 7.8 Hz, 1H), 7.84 (s, 1H), 7.54 (t, J = 7.7 Hz, 1H), 7.35 (d, J = 7.8 Hz, 1H), 4.51 (q, J = 6.8 Hz, 1H), 4.33 (d, J = 10.9 Hz, 1H), 3.53 (s, 3H), 3.37-3.32 (m, 1H), 3.13 (quin, J = 7.6 Hz, 1H), 2.34-2.05 (m, 4H), 1.99-1.86 (m, 5H), 1.85-1.64 (m, 3H), 1.53 (d, J = 6.7 Hz, 3H) |
| | (1S)-N-{(1R)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | ylethyl}-3,3- | | |
| | difluorocyclopentan-1-amine | | |
| C10 | 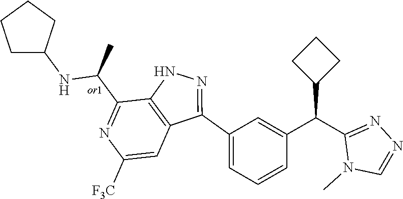 [SMILES: C[C@H](NC1CCCC1)c1nc(C(F)(F)F)cc2c(-c3cccc([C@H](c4nncn4C)C4CCC4)c3)n[nH]c12] | 524.3 | ¹H NMR (600 MHz, DMSO-d₆) δ 8.34 (s, 1H), 8.21 (s, 1H), 7.90- 7.86 (m, 2H), 7.51 (t, J = 8.0 Hz, 1H), 7.33 (d, J = 7.6 Hz, 1H), 4.39-4.33 (m, 2H), 2.80 (t, J = 6.4 Hz, 1H), 2.13-2.09 (m, 1H), 1.90-1.56 (m, 11H), 1.54-1.45 (m, 3H), 1.43 (d, J = 6.9 Hz, 3H), 1.40 (dt, J = 6.9, 4.0 Hz, 4H), 1.16 (dd, J = 7.4, 4.5 Hz, 1H) |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}cyclopentanamine | | |
| C11 | 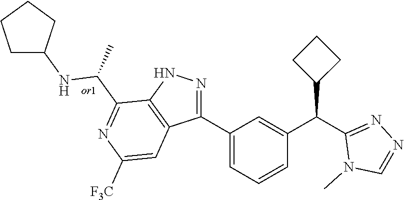 [SMILES: C[C@@H](NC1CCCC1)c1nc(C(F)(F)F)cc2c(-c3cccc([C@H](c4nncn4C)C4CCC4)c3)n[nH]c12] | 524.3 | ¹H NMR (600 MHz, DMSO-d₆) δ 8.34 (s, 1H), 8.20 (s, 1H), 7.91- 7.84 (m, 2H), 7.51 (t, J = 7.8 Hz, 1H), 7.33 (d, J = 8.0 Hz, 1H), 4.40-4.36 (m, 1H), 4.35 (d, J = 10.5 Hz, 1H), 3.26-3.19 (m, 1H), 2.80 (quin, J = 6.4 Hz, 1H), 2.13-2.08 (m, 1H), 1.90-1.57 (m, 10H), 1.55-1.44 (m, 3H), 1.44-1.42 (m, 3H), 1.42-1.28 (m, 4H), 1.17-1.13 (m, 1H) |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}cyclopentanamine | | |
| C12 | 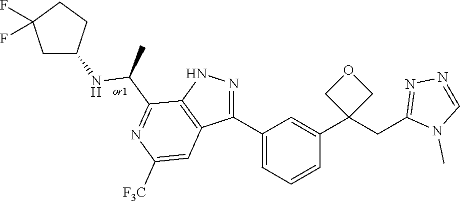 [SMILES: C[C@H](N[C@H]1CCC(F)(F)C1)c1nc(C(F)(F)F)cc2c(-c3cccc(C4(Cc5nncn5C)COC4)c3)n[nH]c12] | 562.3 | ¹H NMR (400 MHz, DMSO-d₆) δ 8.21 (s, 1H), 8.19 (s, 1H), 7.90 (d, J = 8.0 Hz, 1H), 7.61 (s, 1H), 7.49 (t, J = 7.7 Hz, 1H), 7.08 (d, J = 7.70 Hz, 1H), 5.00 (d, J = 6.1 Hz, 2H), 4.95 (d, J = 6.1 Hz, 2H), 4.37 (q, J = 6.4 Hz, 1H), 3.58 (s, 2H), 3.06-3.01 (m, 1H), 2.97 (s, 3H), 2.32-2.24 (m, 1H), 2.18- 1.80 (m, 4H), 1.73 (td, J = 13.0, 6.1 Hz, 1H), 1.51-1.46 (m, 1H), 1.45 (d, J = 6.8 Hz, 3H) |
| | (1S)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}-3,3- | | |
| | difluorocyclopentan-1-amine | | |
| C13 | 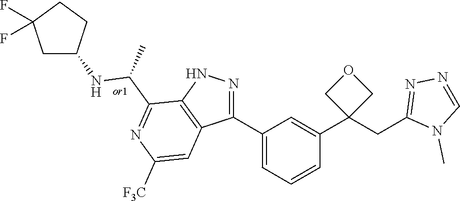 [SMILES: C[C@@H](N[C@H]1CCC(F)(F)C1)c1nc(C(F)(F)F)cc2c(-c3cccc(C4(Cc5nncn5C)COC4)c3)n[nH]c12] | 562.3 | ¹H NMR (400 MHz, DMSO-d₆) δ 8.21 (s, 1H), 8.19 (s, 1H), 7.90 (d, J = 8.0 Hz, 1H), 7.61 (s, 1H), 7.49 (t, J = 7.7 Hz, 1H), 7.08 (d, J = 7.7 Hz, 1H), 5.00 (d, J = 6.1 Hz, 2H), 4.95 (d, J = 6.1 Hz, 2H), 4.42 (q, J = 6.7 Hz, 1H), 3.58 (s, 2H), 3.08-3.02 (m, 1H), 2.97 (s, 3H), 2.23-2.03 (m, 2H), 1.86- 1.99 (m, 2H), 1.86-1.74 (m, 1H), 1.67 (dt, J = 11.7, 7.8 Hz, 1H), 1.45 (d, J = 6.9 Hz, 3H) |
| | (1S)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}-3,3- | | |
| | difluorocyclopentan-1-amine | | |
| C14 | 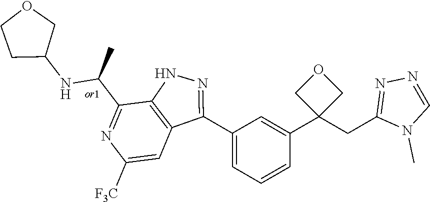 [SMILES: C[C@H](NC1CCOC1)c1nc(C(F)(F)F)cc2c(-c3cccc(C4(Cc5nncn5C)COC4)c3)n[nH]c12] | 526.3 | ¹H NMR (400 MHz, DMSO-d₆) δ 8.20 (s, 1H), 8.19 (s, 1H), 7.89 (d, J = 8.0 Hz, 1H), 7.60 (s, 1H), 7.48 (t, J = 7.7 Hz, 1H), 7.08 (br d, J = 7.6 Hz, 1H), 5.00 (d, J = 6.1 Hz, 2H), 4.95 (d, J = 6.1 Hz, 2H), 4.40 (br s, 1H), 3.58 (s, 2H), 2.97 (s, 3H), 2.79 (br s, 1H), 1.61 (br d, J = 6.4 Hz, 1H), 1.59- 1.26 (m, 9H), 1.23 (br s, 1H) |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}cyclopentanamine | | |
| C15 | 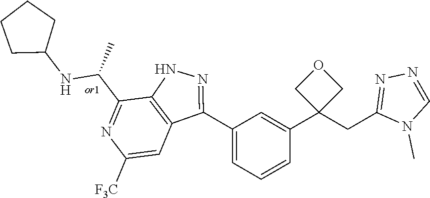 [SMILES: C[C@@H](NC1CCCC1)c1nc(C(F)(F)F)cc2c(-c3cccc(C4(Cc5nncn5C)COC4)c3)n[nH]c12] | 526.3 | ¹H NMR (400 MHz, DMSO-d₆) δ 8.19 (s, 2H), 7.89 (d, J = 7.9 Hz, 1H), 7.60 (s, 1H), 7.48 (t, J = 7.7 Hz, 1H), 7.07 (d, J = 7.9 Hz, 1H), 5.00 (d, J = 6.1 Hz, 2H), 4.95 (d, J = 6.1 Hz, 2H), 4.41 (br s, 1H), 3.58 (br s, 2H), 2.96 (s, 3H), 2.81 (br s, 1H), 1.63 (br s, 1H), 1.57- 1.30 (m, 9H), 1.21 (br s, 1H) |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]cyclopen-7- | | |
| | yl]ethyl}cyclopentanamine | | |
| C16 | 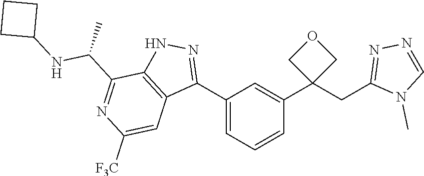 [SMILES: C[C@@H](NC1CCC1)c1nc(C(F)(F)F)cc2c(-c3cccc(C4(Cc5nncn5C)COC4)c3)n[nH]c12] | 512.4 | ¹H NMR (400 MHz, DMSO-d₆) δ 8.19 (s, 1H), 8.19 (s, 1H), 7.89 (d, J = 7.9 Hz, 1H), 7.60 (s, 1H), 7.48 (t, J = 7.7 Hz, 1H), 7.06 (d, J = 7.9 Hz, 1H), 5.00 (d, J = 6.1 Hz, 2H), 4.95 (d, J = 6.1 Hz, 2H), 4.32 (q, J = 6.8 Hz, 1H), 3.58 (s, 2H), 3.10-2.99 (m, 2H), 2.97 (s, 3H), 2.10-1.96 (m, 1H), 1.79- 1.66 (m, 1H), 1.65-1.56 (m, 1H), 1.53-1.33 (m, 6H) |
| | N-{(1R)-1-[3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]cyclopen-7- | | |
| | yl]ethyl}cyclobutanamine | | |
| C17 | 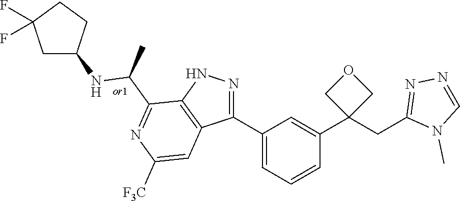 [SMILES: C[C@H](N[C@@H]1CCC(F)(F)C1)c1nc(C(F)(F)F)cc2c(-c3cccc(C4(Cc5nncn5C)COC4)c3)n[nH]c12] | 562.3 | ¹H NMR (400 MHz, DMSO-d₆) δ 8.22 (s, 1H), 8.20 (s, 1H), 7.90 (d, J = 7.9 Hz, 1H), 7.61 (s, 1H), 7.48 (t, J = 7.7 Hz, 1H), 7.08 (d, J = 7.8 Hz, 1H), 5.00 (d, J = 6.1 Hz, 2H), 4.95 (d, J = 6.1 Hz, 2H), 4.94 (d, J = 6.0 Hz, 2H), 4.37 (q, J = 6.7 Hz, 1H), 3.58 (s, 2H), 3.08-3.00 (m, 1H), 2.97 (s, 3H), 2.38-2.24 (m, 2H), 2.14-1.80 (m, 3H), 1.73 (td, J = 12.9, 6.3 Hz, 1H), 1.45 (d, J = 6.7 Hz, 3H) |
| | (1R)-3,3-difluoro-N-{(1ξ)-1-[3- | | |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]cyclopen-7- | | |
| | yl]ethyl}cyclopentane-1-amine | | |
| C18 | 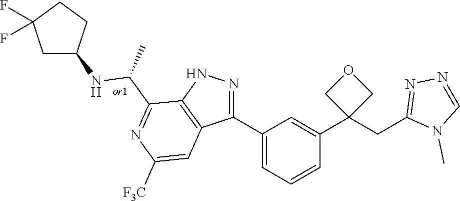 [SMILES: C[C@@H](N[C@@H]1CCC(F)(F)C1)c1nc(C(F)(F)F)cc2c(-c3cccc(C4(Cc5nncn5C)COC4)c3)n[nH]c12] | 562.3 | ¹H NMR (400 MHz, DMSO-d₆) δ 8.21 (s, 1H), 8.19 (s, 1H), 7.90 (d, J = 7.9 Hz, 1H), 7.60 (s, 1H), 7.48 (t, J = 7.8 Hz, 1H), 7.07 (br d, J = 7.9 Hz, 1H), 5.00 (d, J = 6.1 Hz, 2H), 4.95 (d, J = 6.1 Hz, 2H), 4.97-4.91 (m, 2H), 4.42 (q, J = 6.71 Hz, 1H), 3.58 (s, 2H), 3.07-3.02 (m, 1H), 2.97 (s, 3H), 2.25-2.14 (m, 1H), 2.12-2.01 (m, 1H), 2.00-1.90 (m, 2H), 1.88-1.73 (m, 1H), 1.67 (dt, J = 11.9, 8.1 Hz, 1H), 1.45 (d, J = |
| | (1R)-3,3-difluoro-N-{(1ξ)-1-[3- | | 6.8 Hz, 3H) |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]cyclopen-7- | | |
| | yl]ethyl}cyclopentane-1-amine | | |
| C19 | 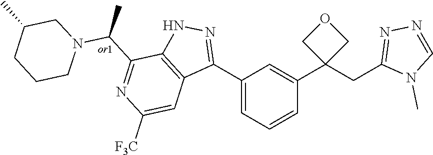 [SMILES: C[C@H]1CCCN([C@@H](C)c2nc(C(F)(F)F)cc3c(-c4cccc(C5(Cc6nncn6C)COC5)c4)n[nH]c23)C1] | 540.5 | ¹H NMR (400 MHz, CD₃OD) δ 8.23 (s, 1H), 8.19 (s, 1H), 7.91 (br d, J = 7.7 Hz, 1H), 7.52 (t, J = 7.8 Hz, 1H), 7.49 (s, 1H), 6.99 (br d, J = 7.8 Hz, 1H), 5.17- 5.07 (m, 4H), 4.14 (q, J = 6.7 Hz, 1H), 3.69 (s, 2H), 3.05 (br d, J = 10.4 Hz, 1H), 2.88 (s, 3H), 2.80- 2.68 (m, 1H), 2.11-1.97 (m, 1H), 1.89 (br t, J = 10.8 Hz, 1H), |
| | 7-{(1ξ)-1-[(3S)-3- | | 1.80-1.63 (m, 4H), 1.54 (d, J = |
| | methylpiperidin-1-yl]ethyl}-3-(3- | | 6.7 Hz, 3H), 0.99-0.84 (m, 1H), |
| | {3-[(4-methyl-4H-1,2,4-triazol-3- | | 0.79 (d, J = 6.5 Hz, 3H) |
| | yl)methyl]oxetan-3-yl}phenyl)-5- | | |
| | (trifluoromethyl)-1H- | | |
| | pyrazolo[3,4-c]pyridine | | |
| C20 | 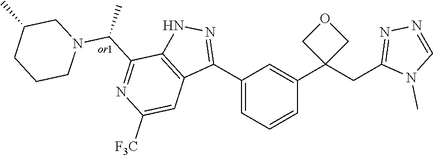 [SMILES: C[C@H]1CCCN([C@H](C)c2nc(C(F)(F)F)cc3c(-c4cccc(C5(Cc6nncn6C)COC5)c4)n[nH]c23)C1] | 540.5 | ¹H NMR (400 MHz, CD₃OD) δ 8.23 (s, 1H), 8.19 (s, 1H), 7.91 (br d, J = 7.7 Hz, 1H), 7.52 (t, J = 7.8 Hz, 1H), 7.49 (s, 1H), 6.99 (br d, J = 7.8 Hz, 1H), 5.17- 5.07 (m, 4H), 4.14 (q, J = 6.7 Hz, 1H), 3.69 (s, 2H), 3.05 (br d, J = 10.4 Hz, 1H), 2.88 (s, 3H), 2.80- 2.68 (m, 1H), 2.11-1.97 (m, 1H), 1.89 (br t, J = 10.8 Hz, 1H), |
| | 7-{(1ξ)-1-[(3S)-3- | | 1.80-1.63 (m, 4H), 1.54 (d, J = |
| | methylpiperidin-1-yl]ethyl}-3-(3- | | 6.7 Hz, 3H), 0.99-0.84 (m, 1H), |
| | {3-[(4-methyl-4H-1,2,4-triazol-3- | | 0.79 (d, J = 6.5 Hz, 3H) |
| | yl)methyl]oxetan-3-yl}phenyl)-5- | | |
| | (trifluoromethyl)-1H- | | |
| | pyrazolo[3,4-c]pyridine | | |
| C21 | 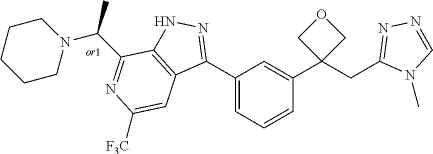 [SMILES: C[C@@H](c1nc(C(F)(F)F)cc2c(-c3cccc(C4(Cc5nncn5C)COC4)c3)n[nH]c12)N1CCCCC1] | 526.3 | ¹H NMR (400 MHz, CD₃OD) δ 8.19 (s, 1H), 8.17 (s, 1H), 7.93 (d, J = 8.0 Hz, 1H), 7.54 (t, J = 7.7 Hz, 1H), 7.50 (s, 1H), 7.01 (d, J = 8.3 Hz, 1H), 5.18-5.11 (m, 4H), 4.14 (q, J = 6.5 Hz, 1H), 3.71 (s, 2H), 2.90 (s, 3H), 2.67 (br s, 2H), 2.51-2.40 (m, 2H), 1.70-1.61 (m, 4H), 1.56 (d, J = 6.8 Hz, 3H), 1.50 (br d, J = 4.3 Hz, |
| | 3-(3-{3-[(4-methyl-4H-1,2,4- | | 2H) |
| | triazol-3-yl)methyl]oxetan-3- | | |
| | yl}phenyl)-7-[(1ξ)-1-(piperidin-1- | | |
| | yl)ethyl]-5-(trifluoromethyl)-1H- | | |
| | pyrazolo[3,4-c]pyridine | | |
| C22 | 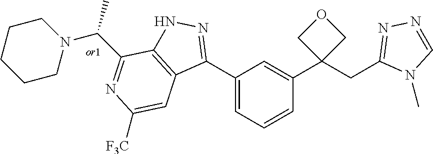 [SMILES: C[C@H](c1nc(C(F)(F)F)cc2c(-c3cccc(C4(Cc5nncn5C)COC4)c3)n[nH]c12)N1CCCCC1] | 526.3 | ¹H NMR (400 MHz, CD₃OD) δ 8.22 (s, 1H), 8.19 (s, 1H), 7.93 (d, J = 8.0 Hz, 1H), 7.55 (t, J = 7.7 Hz, 1H), 7.50 (s, 1H), 7.03 (d, J = 7.8 Hz, 1H), 5.20-5.10 (m, 4H), 4.33 (br s, 1H), 3.72 (s, 2H), 2.93-2.76 (m, 5H), 2.61 (br s, 2H), 1.71 (br d, J = 5.5 Hz, 4H), 1.62 (br d, J = 6.5 Hz, 3H), 1.54 (br s, 2H) |
| | 3-(3-{3-[(4-methyl-4H-1,2,4- | | |
| | triazol-3-yl)methyl]oxetan-3- | | |
| | yl}phenyl)-7-[(1ξ)-1-(piperidin-1- | | |
| | yl)ethyl]-5-(trifluoromethyl)-1H- | | |
| | pyrazolo[3,4-c]pyridine | | |
| C23 | 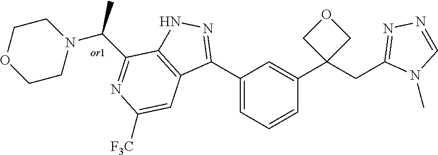 [SMILES: C[C@@H](c1nc(C(F)(F)F)cc2c(-c3cccc(C4(Cc5nncn5C)COC4)c3)n[nH]c12)N1CCOCC1] | 528.4 | ¹H NMR (400 MHz, CD₃OD) δ 8.20 (s, 1H), 8.19 (s, 1H), 7.93 (br d, J = 7.5 Hz, 1H), 7.55 (t, J = 7.7 Hz, 1H), 7.50 (s, 1H), 7.03 (br d, J = 7.8 Hz, 1H), 5.14 (br s, 4H), 4.13 (br s, 1H), 3.77 (br s, 4H), 3.71 (s, 2H), 2.91 (s, 3H), 2.77 (br s, 2H), 2.50 (br s, 2H), 1.59 (br d, J = 5.8 Hz, 3H) |
| | 3-(3-{3-[(4-methyl-4H-1,2,4- | | |
| | triazol-3-yl)methyl]oxetan-3- | | |
| | yl}phenyl)-7-[(1ξ)-1-(morpholin- | | |
| | 4-yl)ethyl]-5-(trifluoromethyl)- | | |
| | 1H-pyrazolo[3,4-c]pyridine | | |
| C24 | 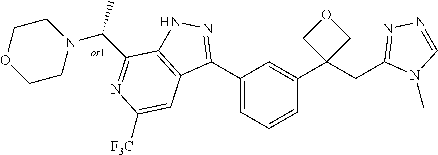 [SMILES: C[C@H](c1nc(C(F)(F)F)cc2c(-c3cccc(C4(Cc5nncn5C)COC4)c3)n[nH]c12)N1CCOCC1] | 528.5 | ¹H NMR (400 MHz, CD₃OD) δ 8.24 (s, 1H), 8.18 (s, 1H), 7.93 (d, J = 7.7 Hz, 1H), 7.55 (t, J = 7.7 Hz, 1H), 7.50 (s, 1H), 7.05 (br d, J = 7.5 Hz, 1H), 5.17- 5.12 (m, 4H), 4.26 (br s, 1H), 3.81 (br s, 4H), 3.71 (s, 2H), 2.93 (s, 3H), 3.07-2.41 (m, 4H), 1.64 (br s, 3H) |
| | 3-(3-{3-[(4-methyl-4H-1,2,4- | | |
| | triazol-3-yl)methyl]oxetan-3- | | |
| | yl}phenyl)-7-[(1ξ)-1-(morpholin- | | |
| | 4-yl)ethyl]-5-(trifluoromethyl)- | | |
| | 1H-pyrazolo[3,4-c]pyridine | | |
| C25 | 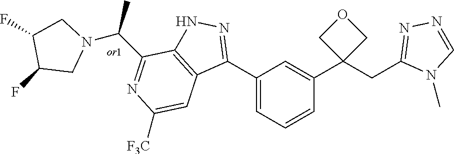 [SMILES: C[C@@H](c1nc(C(F)(F)F)cc2c(-c3cccc(C4(Cc5nncn5C)COC4)c3)n[nH]c12)N1C[C@@H](F)[C@H](F)C1] | 548.4 | ¹H NMR (400 MHz, CD₃OD) δ 8.19 (s, 1H), 8.17 (s, 1H), 7.91 (d, J = 7.8 Hz, 1H), 7.53 (t, J = 7.7 Hz, 1H), 7.48 (s, 1H), 7.00 (d, J = 7.8 Hz, 1H), 5.29-5.17 (m, 1H), 5.15-5.05 (m, 5H), 4.24- 4.13 (m, 1H), 3.69 (s, 2H), 3.12- 2.93 (m, 4H), 2.88 (s, 3H), 1.60 (d, J = 6.8 Hz, 3H) |
| | 7-{(1ξ)-1-[(3R,4R)-3,4- | | |
| | difluoropyrrolidin-1-yl]ethyl}-3- | | |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C26 | 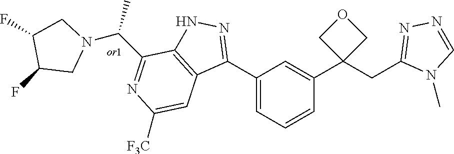 [SMILES: C[C@H](c1nc(C(F)(F)F)cc2c(-c3cccc(C4(Cc5nncn5C)COC4)c3)n[nH]c12)N1C[C@@H](F)[C@H](F)C1] | 548.4 | ¹H NMR (400 MHz, CD₃OD) δ 8.20 (s, 1H), 8.18 (s, 1H), 7.91 (d, J = 7.5 Hz, 1H), 7.52 (t, J = 7.7 Hz, 1H), 7.49 (s, 1H), 6.99 (br d, J = 7.3 Hz, 1H), 5.37- 5.24 (m, 1H), 5.20-5.10 (m, 5H), 4.13 (br s, 1H), 3.70 (s, 2H), 3.43-3.32 (m, 2H), 2.89 (s, 3H), 2.80-2.59 (m, 2H), 1.60 (br d, J = 6.8 Hz, 3H) |
| | 7-{(1ξ)-1-[(3R,4R)-3,4- | | |
| | difluoropyrrolidin-1-ylethyl}-3- | | |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C27 | 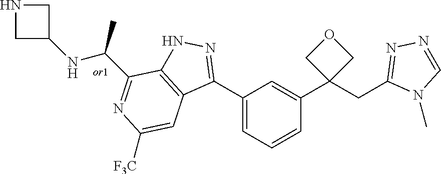 [SMILES: C[C@H](NC1CNC1)c1nc(C(F)(F)F)cc2c(-c3cccc(C4(Cc5nncn5C)COC4)c3)n[nH]c12] | 513.3 | ¹H NMR (400 MHz, CD₃OD) δ 8.20-8.11 (m, 2H), 7.89 (d, J = 7.4 Hz, 1H), 7.51 (t, J = 7.8 Hz, 1H), 7.47 (s, 1H), 6.97 (d, J = 8.0 Hz, 1H), 5.12 (s, 4H), 4.54- 4.37 (m, 1H), 3.77 (s, 1H), 3.69 (s, 4H), 3.57-3.44 (m, 1H), 3.43- 3.34 (m, 1H), 2.87 (d, J = 5.8 Hz, 3H), 1.54 (d, J = 6.6 Hz, 3H) |
| | N-{(1ξ)-1-[3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7- | | |
| | yl]ethyl}azetidin-3-amine | | |
| C28 | 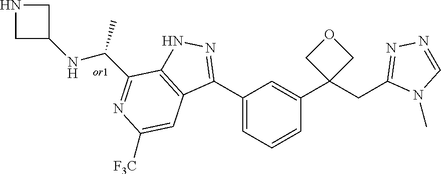 [SMILES: C[C@@H](NC1CNC1)c1nc(C(F)(F)F)cc2c(-c3cccc(C4(Cc5nncn5C)COC4)c3)n[nH]c12] | 513.3 | ¹H NMR (400 MHz, CD₃OD) δ 8.18-8.14 (m, 2H), 7.90 (d, J = 7.7 Hz, 1H), 7.51 (t, J = 7.8 Hz, 1H), 7.47 (s, 1H), 6.98 (d, J = 7.8 Hz, 1H), 5.12 (s, 4H), 4.54- 4.40 (m, 1H), 3.79 (s, 1H), 3.69 (s, 4H), 3.58-3.44 (m, 1H), 3.40- 3.36 (m, 1H), 2.87 (d, J = 6.8 Hz, 3H), 1.54 (d, J = 6.7 Hz, 3H) |
| | N-{(1ξ)-1-[3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7- | | |
| | yl]ethyl}azetidin-3-amine | | |
| C29 | 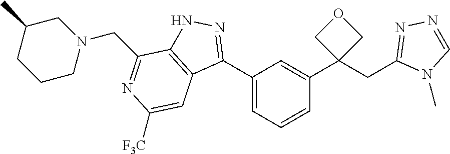 [SMILES: C[C@@H]1CCCN(Cc2nc(C(F)(F)F)cc3c(-c4cccc(C5(Cc6nncn6C)COC5)c4)n[nH]c23)C1] | 526.2 | ¹H NMR (400 MHz, CD₃OD) δ 8.21 (s, 1H), 8.18 (s, 1H), 7.94 (d, J = 7.6 Hz, 1H), 7.54 (t, J = 7.8 Hz, 1H), 7.51 (s, 1H), 7.01 (d, J = 7.9 Hz, 1H), 5.18-5.11 (m, 4H), 4.09 (s, 2H), 3.71 (s, 2H), 2.96-2.85 (m, 5H), 2.14 (br s, 1H), 1.87-1.73 (m, 3H), 1.73- 1.66 (m, 2H), 1.04-0.93 (m, 1H), 0.88 (d, J = 5.9 Hz, 3H) |
| | 7-{[(3R)-3-methylpiperidin-1- | | |
| | yl]methyl}-3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridine | | |
| C30 | 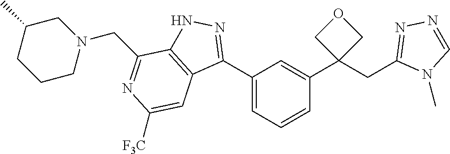 [SMILES: C[C@H]1CCCN(Cc2nc(C(F)(F)F)cc3c(-c4cccc(C5(Cc6nncn6C)COC5)c4)n[nH]c23)C1] | 526.3 | ¹H NMR (400 MHz, CD₃OD) δ 8.21 (s, 1H), 8.18 (s, 1H), 7.93 (d, J = 7.8 Hz, 1H), 7.54 (t, J = 7.8 Hz, 1H), 7.51 (s, 1H), 7.01 (d, J = 7.3 Hz, 1H), 5.19-5.10 (m, 4H), 4.07 (s, 2H), 3.71 (s, 2H), 2.97-2.84 (m, 5H), 2.18-2.08 (m, 1H), 1.86-1.64 (m, 5H), 1.02-0.91 (m, 1H), 0.88 (d, J = 5.9 Hz, 3H) |
| | 7-{[(3S)-3-methylpiperidin-1- | | |
| | yl]methyl}-3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridine | | |
| C31 | 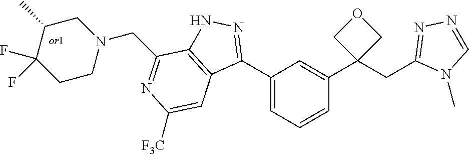 [SMILES: C[C@@H]1CN(Cc2nc(C(F)(F)F)cc3c(-c4cccc(C5(Cc6nncn6C)COC5)c4)n[nH]c23)CCC1(F)F] | 562.3 | ¹H NMR (400 MHz, CD₃OD) δ 8.23 (s, 1H), 8.19 (s, 1H), 7.94 (d, J = 7.8 Hz, 1H), 7.55 (t, J = 7.7 Hz, 1H), 7.50 (s, 1H), 7.02 (d, J = 7.8 Hz, 1H), 5.19-5.08 (m, 4H), 4.18-4.08 (m, 2H), 3.71 (s, 2H), 2.96-2.87 (m, 4H), 2.86- 2.80 (m, 1H), 2.56-246 (m, 1H), 2.30-2.03 (m, 4H), 0.98 (d, J = 6.5 Hz, 3H) |
| | 7-{[(3ξ)-4,4-difluoro-3- | | |
| | methylpiperidin-1-yl]methyl}-3- | | |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C32 | 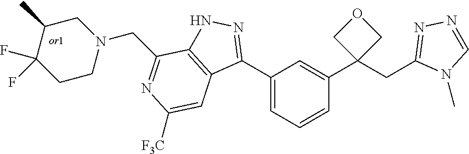 [SMILES: C[C@H]1CN(Cc2nc(C(F)(F)F)cc3c(-c4cccc(C5(Cc6nncn6C)COC5)c4)n[nH]c23)CCC1(F)F] | 562.3 | ¹H NMR (400 MHz, CD₃OD) δ 8.23 (s, 1H), 8.19 (s, 1H), 7.94 (d, J = 8.0 Hz, 1H), 7.55 (t, J = 7.8 Hz, 1H), 7.50 (s, 1H), 7.02 (d, J = 7.8 Hz, 1H), 5.17-5.11 (m, 4H), 4.18-4.08 (m, 2H), 3.71 (s, 2H), 2.96-2.88 (m, 4H), 2.86- 2.79 (m, 1H), 2.51 (br s, 1H), 2.30-2.03 (m, 4H), 0.98 (d, J = 6.5 Hz, 3H) |
| | 7-{[(3ξ)-4,4-difluoro-3- | | |
| | methylpiperidin-1-yl]methyl}-3- | | |
| | (3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C33 | 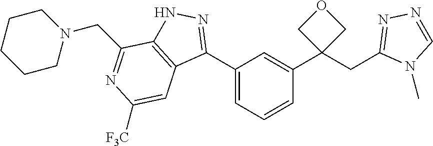 [SMILES: Cn1cnnc1CC1(c2cccc(-c3n[nH]c4c(CN5CCCCC5)nc(C(F)(F)F)cc34)c2)COC1] | 512.4 | ¹H NMR (400 MHz, CD₃OD) δ 8.19 (s, 1H), 8.17 (s, 1H), 7.92 (d, J = 7.8 Hz, 1H), 7.52 (t, J = 7.8 Hz, 1H), 7.49 (s, 1H), 7.00 (d, J = 7.8 Hz, 1H), 5.16-5.09 (m, 4H), 4.06 (s, 2H), 3.69 (s, 2H), 2.88 (s, 3H), 2.57 (br s, 4H), 1.66 (quin, J = 5.5 Hz, 4H), 1.51 (br s, 2H) |
| | 3-(3-{3-[(4-methyl-4H-1,2,4- | | |
| | triazol-3-yl)methyl]oxetan-3- | | |
| | yl}phenyl)-7-[(piperidin-1- | | |
| | yl)methyl]-5-(trifluoromethyl)- | | |
| | 1H-pyrazolo[3,4-c]pyridine | | |
| C34 | 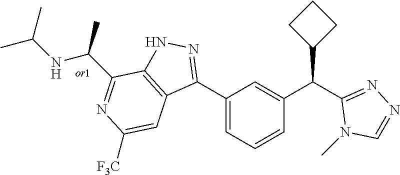 [SMILES: CC(C)N[C@@H](C)c1nc(C(F)(F)F)cc2c(-c3cccc([C@H](c4nncn4C)C4CCC4)c3)n[nH]c12] | 498.1 | ¹H NMR (400 MHz, CD₃OD) δ 8.34 (s, 1H), 8.22 (s, 1H), 7.89 (br s, 2H), 7.50 (t, J = 7.7 Hz, 1H), 7.33 (d, J = 7.8 Hz, 1H), 4.47 (q, J = 6.8 Hz, 1H), 4.37 (d, J = 10.9 Hz, 1H), 3.44 (s, 3H), 3.37-3.32 (m, 1H), 2.12-2.09 (m, 4H), 1.99-1.86 (m, 5H), 1.43 (d, J = 6.8 Hz, 3H), 1.30- 1.20 (m, 1H), 0.99 (d, J = 6 Hz, 3H), 0.90 (d, J = 6 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}propan-2-amine | | |
| C35 | 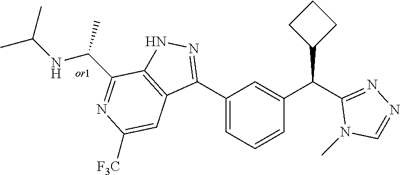 [SMILES: CC(C)N[C@H](C)c1nc(C(F)(F)F)cc2c(-c3cccc([C@H](c4nncn4C)C4CCC4)c3)n[nH]c12] | 498.1 | ¹H NMR (400 MHz, CD₃OD) δ 8.34 (s, 1H), 8.22 (s, 1H), 7.89 (br s, 2H), 7.50 (t, J = 7.7 Hz, 1H), 7.33 (d, J = 7.8 Hz, 1H), 4.47 (q, J = 6.8 Hz, 1H), 4.37 (d, J = 10.9 Hz, 1H), 3.44 (s, 3H), 3.37-3.32 (m, 1H), 2.12-2.09 (m, 4H), 1.99-1.86 (m, 5H), 1.43 (d, J = 6.8 Hz, 3H), 1.30- 1.20 (m, 1H), 0.99 (d, J = 6 Hz, 3H), 0.90 (d, J = 6 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}propan-2-amine | | |
| C36 | 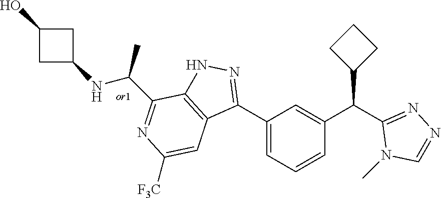 [SMILES: C[C@H](N[C@H]1C[C@@H](O)C1)c1nc(C(F)(F)F)cc2c(-c3cccc([C@H](c4nncn4C)C4CCC4)c3)n[nH]c12] | 526.2 | ¹H NMR (600 MHz, CD₃OD) δ 8.39 (s, 1H), 8.16 (s, 1H), 7.89 (d, J = 7.6 Hz, 1H), 7.85 (s, 1H), 7.55 (t, J = 7.7 Hz, 1H), 7.36 (d, J = 7.8 Hz, 1H), 4.46 (q, J = 6.7 Hz, 1H), 4.38-4.23 (m, 1H), 3.77 (quin, J = 7.4 Hz, 1H), 3.55 (s, 3H), 3.40-3.34 (m, 1H), 2.82- 2.67 (m, 1H), 2.65-2.47 (m, 1H), 2.42-2.25 (m, 1H), 2.21- 2.10 (m, 1H), 2.03-1.88 (m, 4H), 1.85-1.77 (m, 1H), 1.77- |
| | (1S,3s)-3-({(1ξ)-1-[3-{3-[(R)- | | 1.68 (m, 1H), 1.57-1.49 (m, 4H). |
| | cyclobutyl(4-methyl-4H-1,2,4- | | [a]_{D}²² −102.2° (c 0.1, MeOH) |
| | triazol-3-yl)methyl]phenyl}-5- | | |
| | (trifluoromethyl)-1H- | | |
| | pyrazolo[3,4-c]pyridin-7- | | |
| | yl]ethyl}amino)cyclobutan-1-ol | | |
| C37 | 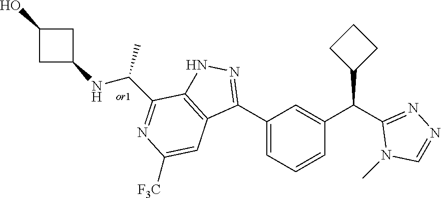 [SMILES: C[C@@H](N[C@H]1C[C@@H](O)C1)c1nc(C(F)(F)F)cc2c(-c3cccc([C@H](c4nncn4C)C4CCC4)c3)n[nH]c12] | 526.2 | ¹H NMR (600 MHz, CD₃OD) δ 8.39 (s, 1H), 8.16 (s, 1H), 7.89 (d, J = 7.8 Hz, 1H), 7.85 (s, 1H), 7.54 (t, J = 7.7 Hz, 1H), 7.36 (d, J = 7.6 Hz, 1H), 4.46 (q, J = 6.8 Hz, 1H), 4.34 (d, J = 10.9 Hz, 1H), 3.81-3.73 (m, 1H), 3.55 (s, 3H), 3.38-3.34 (m, 1H), 2.77- 2.69 (m, 1H), 2.63-2.55 (m, 1H), 2.30 (dquin, J = 3.9, 7.2 Hz, 1H), 2.19-2.11 (m, 1H), 2.03- 1.87 (m, 4H), 1.84-1.77 (m, |
| | (1S,3s)-3-({(1ξ)-1-[3-{3-[(R)- | | 1H), 1.73 (td, J = 8.5, 10.6 Hz, |
| | cyclobutyl(4-methyl-4H-1,2,4- | | 1H), 1.57-1.51 (m, 4H). |
| | triazol-3-yl)methyl]phenyl}-5- | | [a]_{D}²² = −5.6° (c 0.1, MeOH) |
| | (trifluoromethyl)-1H- | | |
| | pyrazolo[3,4-c]pyridin-7- | | |
| | yl]ethyl}amino)cyclobutan-1-ol | | |
| C38 | 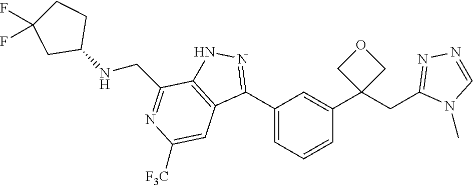 [SMILES: Cn1cnnc1CC1(c2cccc(-c3n[nH]c4c(CN[C@H]5CCC(F)(F)C5)nc(C(F)(F)F)cc34)c2)COC1] | 548.3 | ¹H NMR (400 MHz, CD₃OD) δ 8.21 (s, 1H), 8.18 (s, 1H), 7.93 (br d, J = 7.5 Hz, 1H), 7.54 (br. t, J = 7.8 Hz, 1H), 7.51 (s, 1H), 7.01 (br. d, J = 7.3 Hz, 1H), 5.14 (s, 4H), 4.35 (s, 2H), 3.71 (s, 2H), 3.46-3.39 (m, 1H), 2.90 (s, 3H), 2.53-2.40 (m, 1H), 2.34- 2.10 (m, 2H), 2.10-1.97 (m, 2H), 1.80-1.69 (m, 1H). |
| | (1S)-3,3-difluoro-N-{[3-(3-{3-[(4- | | |
| | methyl-4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7- | | |
| | yl]methyl}cyclopentan-1-amine | | |
| C41 | 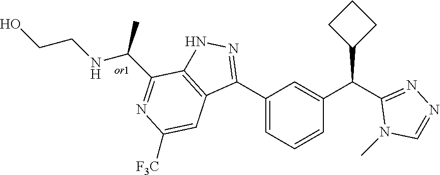 [SMILES: C[C@H](NCCO)c1nc(C(F)(F)F)cc2c(-c3cccc([C@H](c4nncn4C)C4CCC4)c3)n[nH]c12] | 500.2 | ¹H NMR (400 MHz, DMSO-d₆) δ 8.34 (s, 1H), 8.22 (s, 1H), 7.90- 7.84 (m, 2H), 7.51 (t, J = 7.6 Hz, 1H), 7.34 (br d, J = 7.5 Hz, 1H), 4.35 (d, J = 10.5 Hz, 1H), 4.33- 4.28 (m, 1H), 3.49-3.45 (m, 2H), 3.44 (s, 3H), 3.27-3.13 (m, 1H), 2.66-2.59 (m, 1H), 2.40 (td, J = 5.8, 11.8 Hz, 1H), 2.16-2.02 (m, 1H), 1.90-1.67 (m, 6H), 1.46 (d, J = 6.6 Hz, 3H) |
| | 2-({(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)ethanol | | |
| C42 | 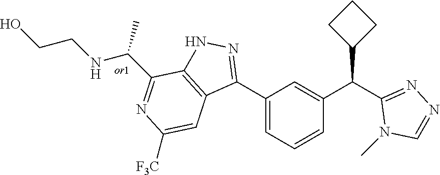 [SMILES: C[C@@H](NCCO)c1nc(C(F)(F)F)cc2c(-c3cccc([C@H](c4nncn4C)C4CCC4)c3)n[nH]c12] | 500.1 | ¹H NMR (400 MHz, DMSO-d₆) δ 8.34 (s, 1H), 8.21 (s, 1H), 7.89- 7.83 (m, 2H), 7.51 (t, J = 7.7 Hz, 1H), 7.34 (br d, J = 7.9 Hz, 1H), 4.34 (d, J = 10.6 Hz, 1H), 4.33- 4.28 (m, 1H), 3.49-3.44 (m, 2H), 3.43 (s, 3H), 3.20 (br d, J = 10.0 Hz, 1H), 2.65-2.58 (m, 1H), 2.39 (td, J = 6.0, 11.9 Hz, 1H), 2.10 (br d, J = 6.4 Hz, 1H), 1.91-1.68 (m, 6H), 1.45 (d, J = |
| | 2-({(1ξ)-1-[3-{3-[(R)- | | 6.8 Hz, 3H) |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)ethanol | | |
| C43 | 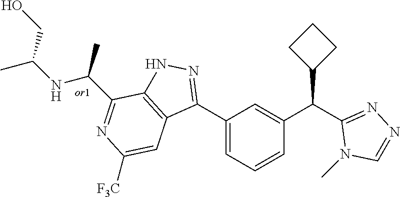 [SMILES: C[C@H](CO)N[C@@H](C)c1nc(C(F)(F)F)cc2c(-c3cccc([C@H](c4nncn4C)C4CCC4)c3)n[nH]c12] | 514.3 | ¹H NMR (400 MHz, CD₃OD) δ 8.37 (s, 1H), 8.15 (s, 1H), 7.87 (d, J = 7.5 Hz, 1H), 7.83 (s, 1H), 7.54 (t, J = 7.7 Hz, 1H), 7.35 (d, J = 7.5 Hz, 1H), 4.33 (d, J = 11.0 Hz, 1H), 3.53 (s, 3H), 3.51-3.44 (m, 1H), 3.43-3.37 (m, 1H), 3.36- 3.32 (m, 1H), 2.67-2.54 (m, 1H), 2.34-2.24 (m, 1H), 2.00- 1.85 (m, 4H), 1.85-1.74 (m, 1H), 1.57 (d, J = 6.8 Hz, 3H), 1.10 (d, J = 6.2 Hz, 3H) |
| | (2ξ)-2-({(1S)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | ylethyl}amino)propan-1-ol | | |
| C44 | 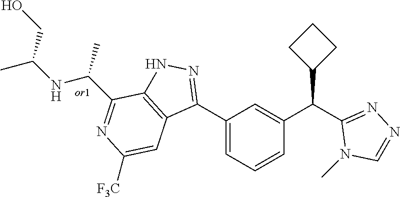 [SMILES: C[C@H](CO)N[C@H](C)c1nc(C(F)(F)F)cc2c(-c3cccc([C@H](c4nncn4C)C4CCC4)c3)n[nH]c12] | 514.3 | ¹H NMR (400 MHz, CD₃OD) δ 8.37 (s, 1H), 8.16 (s, 1H), 7.88 (d, J = 7.5 Hz, 1H), 7.83 (s, 1H), 7.54 (t, J = 7.7 Hz, 1H), 7.35 (d, J = 7.7 Hz, 1H), 4.33 (d, J = 10.8 Hz, 1H), 3.66 (s, 3H), 3.55 (br d, J = 4.8 Hz, 1H), 3.53-3.47 (m, 2H), 2.85-2.72 (m, 1H), 2.35- 2.21 (m, 1H), 2.03-1.85 (m, 4H), 1.84-1.75 (m, 1H), 1.54 (d, J = 6.6 Hz, 3H), 1.01 (d, J = 6.6 Hz, 3H). |
| | (2ξ)-2-({(1S)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)propan-1-ol | | |
| C45 | 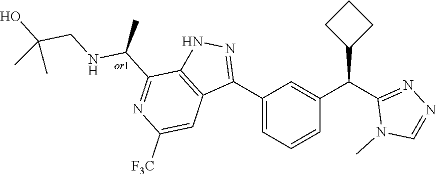 [SMILES: C[C@H](NCC(C)(C)O)c1nc(C(F)(F)F)cc2c(-c3cccc([C@H](c4nncn4C)C4CCC4)c3)n[nH]c12] | 528.5 | ¹H NMR (400 MHz, DMSO-d₆) δ 8.39-8.33 (m, 1H), 8.25 (s, 1H), 8.00-7.82 (m, 2H), 7.60-7.46 (m, 1H), 7.34 (d, J = 7.7 Hz, 1H), 4.61-4.44 (m, 1H), 4.37 (d, J = 10.6 Hz, 1H), 4.34-4.22 (m, 1H), 3.45 (s, 3H), 3.19 (br d, J = 6.6 Hz, 1H), 2.46-2.34 (m, 1H), 2.28-2.16 (m, 1H), 2.16-2.05 (m, 1H), 1.89-1.69 (m, 5H), 1.51 (br d, J = 6.6 Hz, 3H), 1.13-1.03 |
| | 1-({(1ξ)-1-[3-{3-[(R)- | | (m, 6H) |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)-2- | | |
| | methylpropan-2-ol | | |
| C48 | 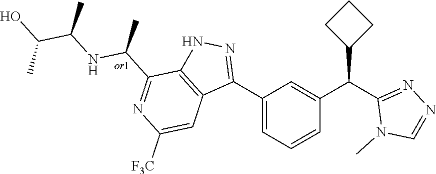 [SMILES: C[C@H](N[C@H](C)[C@H](C)O)c1nc(C(F)(F)F)cc2c(-c3cccc([C@H](c4nncn4C)C4CCC4)c3)n[nH]c12] | 528.2 | ¹H NMR (600 MHz, DMSO-d₆) δ 8.33 (s, 1H), 8.19 (s, 1H), 7.94- 7.80 (m, 2H), 7.49 (t, 1H, J = 8.0 Hz), 7.30 (d, 1H, J = 7.7 Hz), 4.47 (q, 1H, J = 6.8 Hz), 4.35 (d, 1H, J = 10.5 Hz), 3.69-3.66 (m, 1H), 3.44 (s, 3H), 3.23-3.22 (m, 2H), 2.45-2.43 (m, 1H), 2.11-2.10 (m, 1H), 1.84-1.79 (m, 3H), 1.86- 1.70 (m, 1H), 1.43 (d, 3H, J = 6.8 Hz), 0.99 (d, 3H, J = 6.4 Hz), |
| | (2S,3R)-3-({(1ξ)-1-[3-{3-[(R)- | | 0.75 (d, 3H, J = 6.5 Hz) |
| | cyclobutyl(4-methyl-4H-1,2,4- | | [a]_{D}²² −38.5° (c 0.1, MeOH) |
| | triazol-3-yl)methyl]phenyl}-5- | | |
| | (trifluoromethyl)-1H- | | |
| | pyrazolo[3,4-c]pyridin-7- | | |
| | yl]ethyl}amino)butan-2-ol | | |
| C49 | 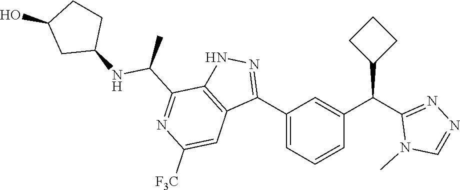 [SMILES: C[C@H](N[C@@H]1CC[C@H](O)C1)c1nc(C(F)(F)F)cc2c(-c3cccc([C@H](c4nncn4C)C4CCC4)c3)n[nH]c12] | 540.5 | ¹H NMR (600 MHz, CD₃OD) δ 8.36 (s, 1H), 8.13 (s, 1H), 7.86 (td, J = 1.0, 7.7 Hz, 1H), 7.83 (t, J = 1.3 Hz, 1H), 7.52 (t, J = 7.6 Hz, 1H), 7.33 (td, J = 1.1, 7.8 Hz, 1H), 4.52 (q, J = 6.8 Hz, 1H), 4.32 (d, J = 10.9 Hz, 1H), 4.12 (tt, J = 5.8, 4.4 Hz, 1H), 3.53 (s, 3H), 3.36-3.31 (m, 1H), 2.99 (quin, J = 6.6 Hz, 1H), 2.31-2.25 (m, 1H), 1.98 (br d, J = 7.1 Hz, 1H), 1.97-1.92 (m, 1H), 1.99- |
| | (1S,3R)-3-({(1S)-1-[3-{3-[(R)- | | 1.90 (m, 2H), 1.97-1.86 (m, 1H), |
| | cyclobutyl(4-methyl-4H-1,2,4- | | 1.88 (br s, 1H), 1.82-1.76 (m, |
| | triazol-3-yl)methyl]phenyl}-5- | | 1H), 1.76-1.71 (m, 1H), 1.76- |
| | (trifluoromethyl)-1H- | | 1.69 (m, 2H), 1.53 (d, J = 6.7 Hz, |
| | pyrazolo[3,4-c]pyridin-7- | | 3H), 1.48-1.42 (m, 1H) |
| | yl]ethyl}amino)cyclopentanol | | |
| C50 | 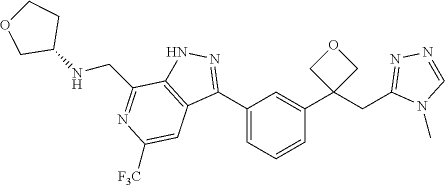 [SMILES: Cn1cnnc1CC1(c2cccc(-c3n[nH]c4c(CN[C@H]5CCOC5)nc(C(F)(F)F)cc34)c2)COC1] | 514.3 | ¹H NMR (400 MHz, CD₃OD) δ 8.21 (s, 1H), 8.17 (s, 1H), 7.93 (d, J = 7.8 Hz, 1H), 7.58-7.48 (m, 2H), 7.01 (d, J = 7.8 Hz, 1H), 5.14 (s, 4H), 4.41-4.32 (m, 2H), 4.06-3.92 (m, 1H), 3.89-3.78 (m, 2H), 3.74 (dd, J = 9.1, 3.9 Hz, 1H), 3.71 (s, 2H), 3.59-3.49 (m, 1H), 2.91 (s, 3H), 2.23-2.14 (m, 1H), 1.96-1.87 (m, 1H). |
| | (3S)-N-{(1S)-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}oxolan-3-amine | | |
| C51 | 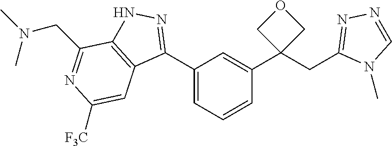 [SMILES: CN(C)Cc1nc(C(F)(F)F)cc2c(-c3cccc(C4(Cc5nncn5C)COC4)c3)n[nH]c12] | 472.4 | ¹H NMR (400 MHz, DMSO-d₆) δ 8.25 (s, 1H), 8.19 (s, 1H), 7.90 (d, J = 7.8 Hz, 1H), 7.59 (s, 1H), 7.49 (t, J = 7.7 Hz, 1H), 7.10 (d, J = 7.7 Hz, 1H), 5.04-4.98 (m, 2H), 4.97-4.93 (m, 2H), 3.95 (s, 2H), 3.58 (s, 2H), 2.98 (s, 3H), 2.27 (s, 6H). |
| | N,N-dimethyl-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methanamine | | |
| C52 | 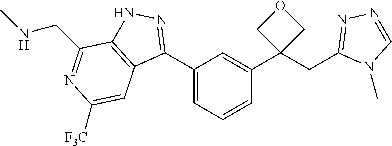 [SMILES: CNCc1nc(C(F)(F)F)cc2c(-c3cccc(C4(Cc5nncn5C)COC4)c3)n[nH]c12] | 458.3 | ¹H NMR (400 MHz, CD₃OD) δ 8.32 (s, 1H), 8.15 (s, 1H), 7.92 (br d, J = 7.6 Hz, 1H), 7.55 (br t, J = 7.7 Hz, 1H), 7.49 (s, 1H), 7.07 (br d, J = 7.9 Hz, 1H), 5.15- 5.09 (m, 4H), 4.76 (s, 2H), 3.70 (s, 2H), 2.93 (s, 3H), 2.91 (s, 3H). |
| | N-methyl-1-[3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methanamine | | |
| C53 | 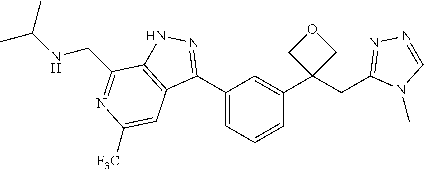 [SMILES: CC(C)NCc1nc(C(F)(F)F)cc2c(-c3cccc(C4(Cc5nncn5C)COC4)c3)n[nH]c12] | 486.3 | ¹H NMR (400 MHz, DMSO-d₆) δ 8.16 (s, 1H), 7.96-7.75 (m, 2H), 7.59 (s, 1H), 7.31 (br t, J = 7.6 Hz, 1H), 6.63 (br d, J = 6.5 Hz, 1H), 5.00 (d, J = 5.9 Hz, 2H), 4.93 (d, J = 5.9 Hz, 2H), 4.19 (br s, 2H), 3.76-3.48 (m, 2H), 2.86 (s, 3H), 2.80-2.74 (m, 1H), 1.04 (br d, J = 5.9 Hz, 6H). |
| | N-{[3-(3-{3-[(4-methyl-4H- | | |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}propan-2-amine | | |
| C54 | 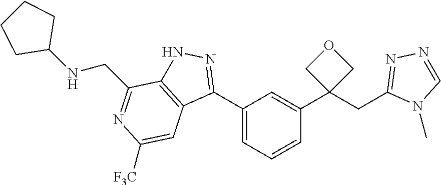 [SMILES: Cn1cnnc1CC1(c2cccc(-c3n[nH]c4c(CNC5CCCC5)nc(C(F)(F)F)cc34)c2)COC1] | 512.4 | ¹H NMR (400 MHz, DMSO-d₆) δ 8.22 (s, 1H), 8.18 (s, 1H), 7.90 (d, J = 7.9 Hz, 1H), 7.60 (s, 1H), 7.48 (t, J = 7.8 Hz, 1H), 7.08 (d, J = 7.7 Hz, 1H), 5.03-4.90 (m, 4H), 4.25 (s, 2H), 3.58 (s, 2H), 3.06 (quin, J = 6.1 Hz, 1H), 2.97 (s, 3H), 1.78-1.56 (m, 4H), 1.53- 1.33 (m, 4H). |
| | N-{[3-(3-{3-[(4-methyl-4H- | | |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}cyclopentanamine | | |
| C55 | 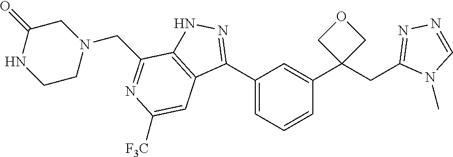 [SMILES: Cn1cnnc1CC1(c2cccc(-c3n[nH]c4c(CN5CCNC(=O)C5)nc(C(F)(F)F)cc34)c2)COC1] | | ¹H NMR (400 MHz, DMSO-d₆) δ 8.27 (s, 1 H), 8.19 (s, 1 H), 7.90 (d, J = 7.25 Hz, 1 H), 7.77 (s, 1 H), 7.59 (s, 1 H), 7.49 (t, J = 7.75 Hz, 1 H), 7.10 (d, J = 8.25 Hz, 1 H), 5.01-4.97 (m, 2 H), 4.97-4.92 (m, 2 H), 4.08 (s, 2 H), 3.58 (s, 2 H), 3.18-3.14 (m, 2 H), 3.10 (s, 2 H), 2.97 (s, 3 H), 2.46-2.42 (m, 2 H). |
| | 4-{[3-(3-{3-[(4-methyl-4H- | | |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}piperazin-2-one | | |
| C56 | 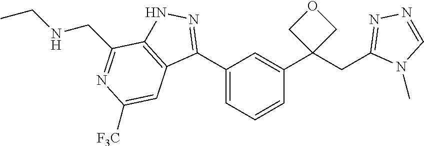 [SMILES: CCNCc1nc(C(F)(F)F)cc2c(-c3cccc(C4(Cc5nncn5C)COC4)c3)n[nH]c12] | 472.4 | ¹H NMR (400 MHz, CD₃OD) δ 8.50 (s, 1H), 8.31 (s, 1H), 8.15 (s, 1H), 7.92 (d, J = 7.8 Hz, 1H), 7.55 (t, J = 7.8 Hz, 1H), 7.49 (s, 1H), 7.06 (d, J = 7.8 Hz, 1H), 5.18-5.09 (m, 4H), 4.74 (s, 2H), 3.70 (s, 2H), 3.24 (q, J = 7.2 Hz, 2H), 2.93 (s, 3H), 1.41 (t, J = 7.3 Hz, 3H). |
| | N-{[3-(3-{3-[(4-methyl-4H- | | |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}ethanamine | | |
| C57 | 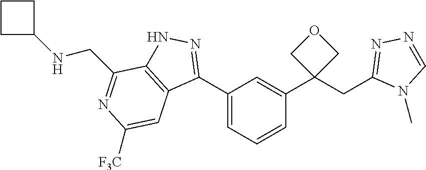 [SMILES: Cn1cnnc1CC1(c2cccc(-c3n[nH]c4c(CNC5CCC5)nc(C(F)(F)F)cc34)c2)COC1] | 498.3 | ¹H NMR (400 MHz, CD₃OD) δ 8.22 (s, 1H), 8.17 (s, 1H), 7.93 (br d, J = 7.8 Hz, 1H), 7.54 (t, J = 7.8 Hz, 1H), 7.51 (s, 1H), 7.02 (br d, J = 7.7 Hz, 1H), 5.22-5.06 (m, 4H), 4.32 (s, 2H), 3.71 (s, 2H), 3.49 (quin, J = 7.8 Hz, 1H), 2.91 (s, 3H), 2.21 (q, J = 7.9 Hz, 2H), 1.96-1.85 (m, 2H), 1.85- 1.62 (m, 2H). |
| | N-{[3-(3-{3-[(4-methyl-4H- | | |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}cyclobutanamine | | |
| C58 | 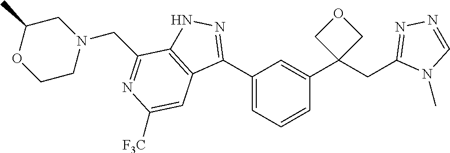 [SMILES: C[C@H]1CN(Cc2nc(C(F)(F)F)cc3c(-c4cccc(C5(Cc6nncn6C)COC5)c4)n[nH]c23)CCO1] | 528.3 | ¹H NMR (400 MHz, CD₃OD) δ 8.21 (s, 1 H), 8.17 (s, 1 H), 7.93 (d, J = 8.1 Hz, 1 H), 7.59-7.44 (m, 2 H), 7.02 (d, J = 8.0 Hz, 1 H), 5.18-5.10 (m, 4 H), 4.08 (s, 2 H), 3.87-3.73 (m, 3 H), 3.71 (s, 2 H), 2.91 (s, 3 H), 2.85-2.72 (m, 2 H), 2.34 (td, J = 11.3, 3.5 Hz, 1 H), 2.02 (t, J = 10.6 Hz, 1 H), 1.12 (d, |
| | 7-{[(2S)-2-methylmorpholin-4- | | J = 6.4 Hz, 3 H). |
| | yl]methyl}-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C59 | 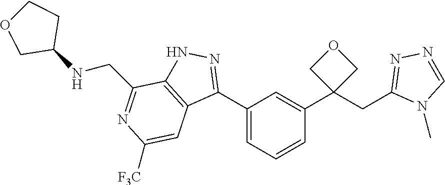 [SMILES: Cn1cnnc1CC1(c2cccc(-c3n[nH]c4c(CN[C@@H]5CCOC5)nc(C(F)(F)F)cc34)c2)COC1] | 514.3 | ¹H NMR (400 MHz, DMSO-d₆) δ 8.25 (s, 1H), 8.19 (s, 1H), 7.91 (br d, J = 7.8 Hz, 1H), 7.60 (s, 1H), 7.49 (t, J = 7.2 Hz, 1H), 7.10 (br d, J = 7.6 Hz, 1H), 5.04-4.97 (m, 2H), 4.95 (d, J = 6.0 Hz, 2H), 4.32-4.20 (m, 2H), 3.79 (q, J = 7.4 Hz, 1H), 3.74- 3.63 (m, 3H), 3.59 (s, 3H), 3.52 (br dd, J = 4.1, 8.8 Hz, 2H), 2.98 (s, 4H), 1.96 (br dd, J = 7.4, 12.4 Hz, 2H), 1.76 (dt, J = 5.5, |
| | (3R)-N-{[3-(3-{3-[(4-methyl-4H- | | 11.9 Hz, 1H). |
| | 1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}oxolan-3-amine | | |
| C60 | 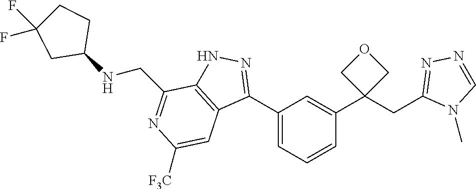 [SMILES: Cn1cnnc1CC1(c2cccc(-c3n[nH]c4c(CN[C@@H]5CCC(F)(F)C5)nc(C(F)(F)F)cc34)c2)COC1] | 548.4 | ¹H NMR (400 MHz, DMSO-d₆) δ 8.25 (s, 1 H), 8.19 (s, 1 H), 7.91 (br d, J = 8.0 Hz, 1 H), 7.60 (s, 1 H), 7.49 (t, J = 7.8 Hz, 1 H), 7.10 (br d, J = 7.6 Hz, 1 H), 5.11- 4.94 (m, 4 H), 4.24 (s, 2 H), 3.59 (s, 2 H), 2.98 (s, 3 H), 2.28-2.14 (m, 2 H), 2.09-1.90 (m, 4 H), 1.68-1.65 (m, 1 H). |
| | (1R)-3,3-difluoro-N-{[3-(3-{3- | | |
| | [(4-methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}cyclopentan-1-amine | | |
| C61 | 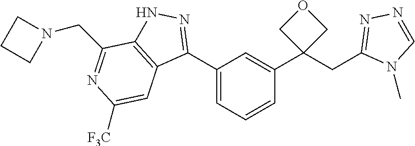 [SMILES: Cn1cnnc1CC1(c2cccc(-c3n[nH]c4c(CN5CCC5)nc(C(F)(F)F)cc34)c2)COC1] | 484.4 | ¹H NMR (400 MHz, CD₃OD) δ 8.45 (s, 1H), 8.29 (s, 1H), 8.15 (s, 1H), 7.91 (d, J = 8.0 Hz, 1H), 7.54 (t, J = 7.8 Hz, 1H), 7.50 (s, 1H), 7.05 (d, J = 7.8 Hz, 1H), 5.15-5.08 (m, 4H), 4.82 (s, 2H), 4.17 (t, J = 7.9 Hz, 4H), 3.70 (s, 2H), 2.96 (s, 3H), 2.54 (quin, J = 8.0 Hz, 2H). |
| | 7-[(azetidin-1-yl)methyl]-3-(3- | | |
| | {3-[(4-methyl-4H-1,2,4-triazol- | | |
| | 3-yl)methyl]oxetan-3- | | |
| | yl}phenyl)-5-(trifluoromethyl)- | | |
| | 1H-pyrazolo[3,4-c]pyridine | | |
| C62 | 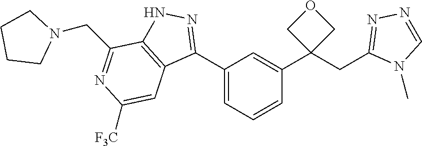 [SMILES: Cn1cnnc1CC1(c2cccc(-c3n[nH]c4c(CN5CCCC5)nc(C(F)(F)F)cc34)c2)COC1] | 498.3 | ¹H NMR (400 MHz, CD₃OD) δ 8.45 (br s, 1H), 8.29 (s, 1H), 8.16 (s, 1H), 7.92 (d, J = 8.0 Hz, 1H), 7.54 (t, J = 7.8 Hz, 1H), 7.48 (s, 1H), 7.04 (d, J = 8.3 Hz, 1H), 5.16-5.09 (m, 4H), 4.72 (s, 2H), 3.70 (s, 2H), 3.26 (br s, 4H), 2.91 (s, 3H), 2.1-2.02 (m, 4H). |
| | 3-(3-{3-[(4-methyl-4H-1,2,4- | | |
| | triazol-3-yl)methyl]oxetan-3- | | |
| | yl}phenyl)-7-[(pyrrolidin-1- | | |
| | yl)methyl]-5-(trifluoromethyl)- | | |
| | 1H-pyrazolo[3,4-c]pyridine | | |
| C63 | 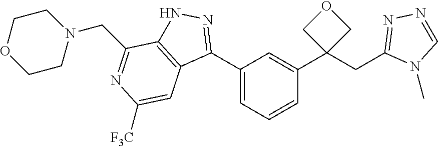 [SMILES: Cn1cnnc1CC1(c2cccc(-c3n[nH]c4c(CN5CCOCC5)nc(C(F)(F)F)cc34)c2)COC1] | 514.3 | ¹H NMR (400 MHz, CD₃OD) δ 8.23 (s, 1H), 8.17 (s, 1H), 7.92 (d, J = 7.8 Hz, 1H), 7.53 (t, J = 7.7 Hz, 1H), 7.48 (s, 1H), 7.01 (d, J = 8.0 Hz, 1H), 5.18-5.08 (m, 4H), 4.18 (br s, 2H), 3.84-3.73 (m, 4H), 3.70 (s, 2H), 2.89 (s, 3H), 2.75-2.58 (m, 4H). |
| | 3-(3-{3-[(4-methyl-4H-1,2,4- | | |
| | triazol-3-yl)methyl]oxetan-3- | | |
| | yl}phenyl)-7-[(morpholin-4- | | |
| | yl)methyl]-5-(trifluoromethyl)- | | |
| | 1H-pyrazolo[3,4-c]pyridine | | |
| C64 | 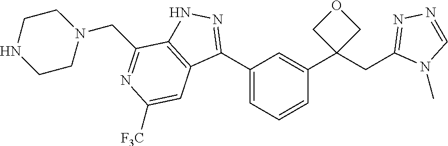 [SMILES: Cn1cnnc1CC1(c2cccc(-c3n[nH]c4c(CN5CCNCC5)nc(C(F)(F)F)cc34)c2)COC1] | 513.3 | ¹H NMR (400 MHz, DMSO-d₆) δ 8.24 (s, 1H), 8.18 (s, 1H), 7.90 (d, J = 7.9 Hz, 1H), 7.59 (s, 1H), 7.48 (t, J = 7.7 Hz, 1H), 7.09 (d, J = 7.7 Hz, 1H), 4.98 (d, J = 6.0 Hz, 2H), 4.93 (d, J = 6.0 Hz, 2H), 3.97 (s, 2H), 3.57 (s, 2H), 2.97 (s, 3H), 2.74 (br t, J = 4.5 Hz, 4H), 2.43 (br s, 4H). |
| | 3-(3-{3-[(4-methyl-4H-1,2,4- | | |
| | triazol-3-yl)methyl]oxetan-3- | | |
| | yl}phenyl)-7-[(piperazin-1- | | |
| | yl)methyl]-5-(trifluoromethyl)- | | |
| | 1H-pyrazolo[3,4-c]pyridine | | |
| C65 | 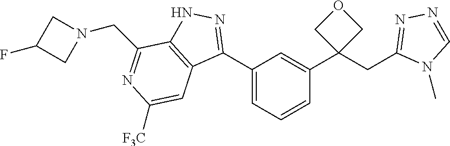 [SMILES: Cn1cnnc1CC1(c2cccc(-c3n[nH]c4c(CN5CC(F)C5)nc(C(F)(F)F)cc34)c2)COC1] | 502.1 | ¹H NMR (400 MHz, DMSO-d₆) δ 8.25 (s, 1H), 8.19 (s, 1H), 7.90 (d, J = 7.7 Hz, 1H), 7.59 (s, 1H), 7.49 (t, J = 7.7 Hz, 1H), 7.10 (br d, J = 8.3 Hz, 1H), 5.34-5.12 (m, 1H), 4.99 (d, J = 6.0 Hz, 2H), 4.95 (d, J = 6.0 Hz, 2H), 4.19 (s, 2H), 3.75-3.65 (m, 2H), 3.58 (s, 2H), 2.98 (s, 3H). |
| | 7-[(3-fluoroazetidin-1- | | |
| | yl)methyl]-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C66 | 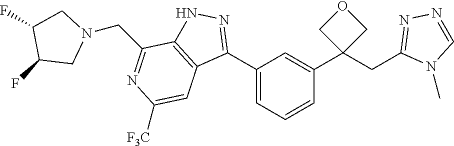 [SMILES: Cn1cnnc1CC1(c2cccc(-c3n[nH]c4c(CN5C[C@@H](F)[C@H](F)C5)nc(C(F)(F)F)cc34)c2)COC1] | 534.3 | ¹H NMR (400 MHz, CD₃OD) δ 8.21 (s, 1H), 8.16 (s, 1H), 7.91 (d, J = 7.7 Hz, 1H), 7.62-7.38 (m, 2H), 6.99 (br d, J = 8.1 Hz, 1H), 5.31-5.20 (m, 1H), 5.17- 5.05 (m, 5H), 4.35 (d, J = 14.1 Hz, 1H), 4.17 (d, J = 14.1 Hz, 1H), 3.69 (s, 2H), 3.28-3.14 (m, 2H), 2.98-2.80 (m, 5H). |
| | 7-{[(3R,4R)-3,4- | | |
| | difluoropyrrolidin-1-yl]methyl}- | | |
| | 3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C67 | 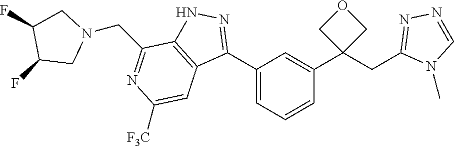 [SMILES: Cn1cnnc1CC1(c2cccc(-c3n[nH]c4c(CN5C[C@@H](F)[C@@H](F)C5)nc(C(F)(F)F)cc34)c2)COC1] | 534.3 | ¹H NMR (400 MHz, CD₃OD) δ 8.28 (s, 1H), 8.23 (s, 1H), 8.07 (s, 1H), 7.92 (d, J = 7.8 Hz, 1H), 7.54 (t, J = 7.8 Hz, 1H), 7.49 (s, 1H), 7.05 (br d, J = 7.8 Hz, 1H), 5.45-5.20 (m, 2H), 5.12 (s, 4H), 4.67 (br s, 1H), 3.71 (s, 2H), 3.51 (br d, J = 18.3 Hz, 3H), 3.31 (td, J = 1.6, 3.3 Hz, 6H), 2.94 (s, 3H). |
| | 7-{[(3R,4S)-3,4- | | |
| | difluoropyrrolidin-1-yl]methyl}- | | |
| | 3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C68 | 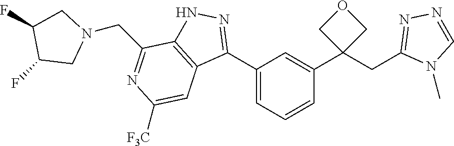 [SMILES: Cn1cnnc1CC1(c2cccc(-c3n[nH]c4c(CN5C[C@H](F)[C@@H](F)C5)nc(C(F)(F)F)cc34)c2)COC1] | 534.3 | ¹H NMR (400 MHz, CD₃OD) δ 8.21 (s, 1H), 8.15 (s, 1H), 7.91 (br d, J = 7.8 Hz, 1H), 7.56-7.46 (m, 2H), 6.99 (br d, J = 8.0 Hz, 1H), 5.29-5.20 (m, 1H), 5.07- 5.18 (m, 5H), 4.35 (d, J = 14.1 Hz, 1H), 4.17 (d, J = 14.1 Hz, 1H), 3.69 (s, 2H), 3.28-3.14 (m, 2H), 2.97-2.82 (m, 5H). |
| | 7-{[(3S,4S)-3,4- | | |
| | difluoropyrrolidin-1-yl]methyl}- | | |
| | 3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridine | | |
| C69 | 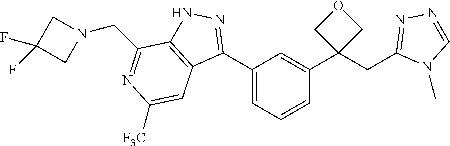 [SMILES: Cn1cnnc1CC1(c2cccc(-c3n[nH]c4c(CN5CC(F)(F)C5)nc(C(F)(F)F)cc34)c2)COC1] | 520.3 | ¹H NMR (400 MHz, CD₃OD) δ 8.22 (s, 1H), 8.18 (s, 1H), 7.92 (d, J = 7.8 Hz, 1H), 7.54 (t, J = 7.8 Hz, 1H), 7.50 (s, 1H), 7.01 (d, J = 7.8 Hz, 1H), 5.17-5.10 (m, 4H), 4.35 (s, 2H), 3.85 (t, J = 12.2 Hz, 4H), 3.71 (s, 2H), 2.90 (s, 3H). |
| | 7-[(3,3-difluoroazetidin-1- | | |
| | yl)methyl]-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C70 | 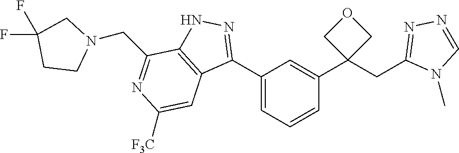 [SMILES: Cn1cnnc1CC1(c2cccc(-c3n[nH]c4c(CN5CCC(F)(F)C5)nc(C(F)(F)F)cc34)c2)COC1] | 534.3 | ¹H NMR (400 MHz, CD₃OD) δ 8.25 (d, J = 2.3 Hz, 2H), 7.92 (d, J = 7.8 Hz, 1H), 7.53 (t, J = 7.8 Hz, 1H), 7.50 (s, 1H), 7.02 (d, J = 7.5 Hz, 1H), 5.12 (s, 4H), 4.35 (br s, 2H), 3.71 (s, 2H), 3.25- 3.00 (m, 4H), 2.92 (s, 3H), 2.40 (tt, J = 7.2, 14.5 Hz, 2H). |
| | 7-[(3,3-difluoropyrrolidin-1- | | |
| | yl)methyl]-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C71 | 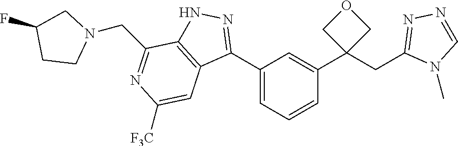 [SMILES: Cn1cnnc1CC1(c2cccc(-c3n[nH]c4c(CN5CC[C@@H](F)C5)nc(C(F)(F)F)cc34)c2)COC1] | 516.3 | ¹H NMR (400 MHz, CD₃OD) δ 8.21 (s, 1H), 8.16 (s, 1H), 7.91 (d, J = 7.7 Hz, 1H), 7.60-7.39 (m, 2H), 7.00 (br d, J = 8.3 Hz, 1H), 5.35-5.16 (m, 1H), 5.15- 5.10 (m, 4H), 4.60 (br s, 2H), 4.40-4.20 (m, 2H), 3.69 (s, 2H), 3.10-2.95 (m, 2H), 2.89 (s, 3H), 2.39-2.01 (m, 2H). |
| | 7-{[(3R)-3-fluoropyrrolidin-1- | | |
| | yl]methyl}-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C72 | 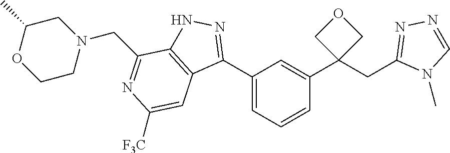 [SMILES: C[C@@H]1CN(Cc2nc(C(F)(F)F)cc3c(-c4cccc(C5(Cc6nncn6C)COC5)c4)n[nH]c23)CCO1] | 528.3 | ¹H NMR (400 MHz, CD₃OD) δ 8.22 (s, 1H), 8.18 (s, 1H), 7.93 (d, J = 7.7 Hz, 1H), 7.58-7.46 (m, 2H), 7.02 (d, J = 7.7 Hz, 1H), 5.17-5.08 (m, 4H), 4.08 (s, 2H), 3.88-3.67 (m, 5H), 2.91 (s, 3H), 2.86-2.70 (m, 2H), 2.34 (td, J = 11.3, 3.2 Hz, 1H), 2.09-1.95 (m, 1H), 1.12 (d, J = 6.2 Hz, 3H). |
| | 7-{[(2R)-2-methylmorpholin-4- | | |
| | yl]methyl}-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C73 | 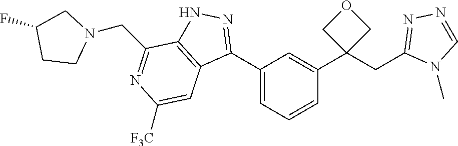 [SMILES: Cn1cnnc1CC1(c2cccc(-c3n[nH]c4c(CN5CC[C@H](F)C5)nc(C(F)(F)F)cc34)c2)COC1] | 516.3 | ¹H NMR (400 MHz, CD₃OD) δ 8.21 (s, 1H), 8.17 (s, 1H), 7.93 (d, J = 8.0 Hz, 1H), 7.67-7.39 (m, 2H), 7.01 (d, J = 7.7 Hz, 1H), 5.35-5.16 (m, 1H), 5.16-5.11 (m, 4H), 4.38-4.19 (m, 2H), 3.71 (s, 2H), 2.84-3.07 (m, 6H), 2.61- 2.71 (m, 1H), 2.00-2.37 (m, 2H). |
| | 7-{[(3S)-3-fluoropyrrolidin-1- | | |
| | yl]methyl}-3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridine | | |
| C74 | 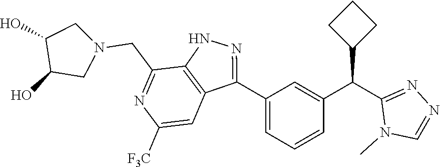 [SMILES: Cn1cnnc1[C@@H](c1cccc(-c2n[nH]c3c(CN4C[C@@H](O)[C@H](O)C4)nc(C(F)(F)F)cc23)c1)C1CCC1] | 528.2 | ¹H NMR (400 MHz, DMSO-d₆) δ 8.34 (s, 1H), 8.27 (s, 1H), 7.91 (d, J = 2.0 Hz, 2H), 7.49 (t, J = 7.5 Hz, 1H), 7.33 (d, J = 6.0 Hz, 1H), 5.00 (br s, 1H), 4.38 (d, J = 10.4 Hz, 1H), 4.15 (s, 2H), 3.89 (br s, 2H), 3.44 (s, 3H), 3.19- 3.18 (m, 2H), 2.96-2.92 (m, 2H), 2.15-2.05 (m, 1H), 1.83-1.79 (m, 6H). |
| | (3R,4R)-1-{[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}pyrrolidine-3,4-diol | | |
| C75 | 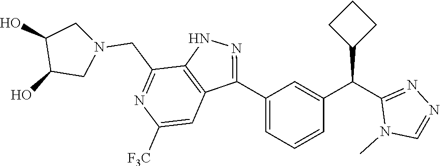 [SMILES: Cn1cnnc1[C@@H](c1cccc(-c2n[nH]c3c(CN4C[C@@H](O)[C@@H](O)C4)nc(C(F)(F)F)cc23)c1)C1CCC1] | 528.2 | ¹H NMR (400 MHz, DMSO-d₆) δ 8.34 (s, 1H), 8.26 (s, 1H), 7.90- 7.89 (m, 2H), 7.50 (t, J = 4.0 Hz, 1H), 7.33 (d, J = 7.6 Hz, 1H), 4.64 (br s, 1H), 4.38 (d, J = 10.4 Hz, 1H), 4.14 (s, 2H), 4.01 (br s, 2H), 3.44 (s, 3H), 2.90-2.87 (m, 2H), 2.10-2.00 (m, 1H), 1.86- 1.72 (m, 6H). |
| | (3R,4S)-1-{[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}pyrrolidine-3,4-diol | | |
| C76 | 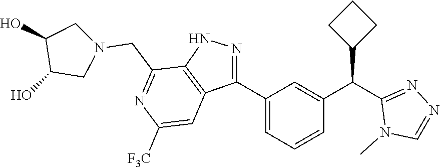 [SMILES: Cn1cnnc1[C@@H](c1cccc(-c2n[nH]c3c(CN4C[C@H](O)[C@@H](O)C4)nc(C(F)(F)F)cc23)c1)C1CCC1] | 528.2 | ¹H NMR (400 MHz, DMSO-d₆) δ 8.34 (s, 1H), 8.27 (s, 1H), 7.91- 7.89 (m, 2H), 7.51 (t, J = 7.6 Hz, 1H), 7.33 (d, J = 7.6 Hz, 1H), 4.99 (br s, 1H), 4.37 (d, J = 10.4 Hz, 1H), 4.16 (s, 2H), 3.89 (br s, 2H), 3.44 (s, 3H), 3.21-3.19 (m, 1H), 2.96-2.92 (m, 2H), 2.47- 2.44 (m, 1H), 2.10-2.00 (m, 1H), 1.86-1.74 (m, 6H). |
| | (3S,4S)-1-{[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}pyrrolidine-3,4-diol | | |
| C77 | 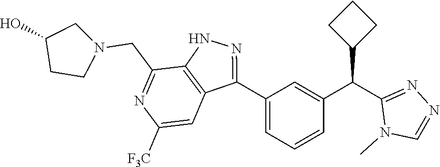 [SMILES: Cn1cnnc1[C@@H](c1cccc(-c2n[nH]c3c(CN4CC[C@H](O)C4)nc(C(F)(F)F)cc23)c1)C1CCC1] | 512.2 | ¹H NMR (400 MHz, DMSO-d₆) δ 8.34 (s, 1H), 8.26 (s, 1H), 7.90 (d, J = 2.0 Hz, 2H), 7.49 (t, J = 4.0 Hz, 1H), 7.34 (d, J = 7.6 Hz, 1H), 4.38 (d, J = 10.4 Hz, 1H), 4.22-4.20 (m, 1H), 4.13 (s, 2H), 3.45 (s, 3H), 3.24-3.15 (m, 2H), 2.77-2.70 (m, 2H), 2.09-2.07 (m, 2H), 1.84-1.57 (m, 7H). |
| | (3S)-1-{[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}pyrrolidin-3-ol | | |
| C78 | 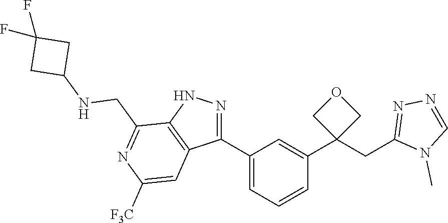 [SMILES: Cn1cnnc1CC1(c2cccc(-c3n[nH]c4c(CNC5CC(F)(F)C5)nc(C(F)(F)F)cc34)c2)COC1] | 534.0 | ¹H NMR (400 MHz, DMSO-d₆) δ 8.24 (s, 1H), 8.18 (s, 1H), 7.90 (d, J = 4.0 Hz, 2H), 7.59 (s, 1H), 7.48 (t, J = 8.0 Hz, 1H), 7.10 (d, J = 7.2 Hz, 1H), 5.00-4.93 (m, 4H), 4.19 (s, 2H), 3.57 (s, 2H), 2.97 (s, 3H), 2.71-2.66 (m, 2H), 2.44-2.36 (m, 2H). |
| | 3,3-difluoro-N-{[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]methyl}cyclobutan-1-amine | | |
| C79 | 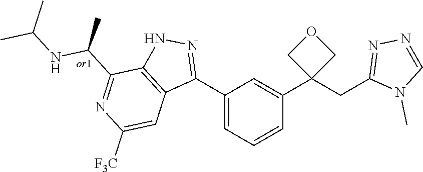 [SMILES: CC(C)N[C@@H](C)c1nc(C(F)(F)F)cc2c(-c3cccc(C4(Cc5nncn5C)COC4)c3)n[nH]c12] | 500.4 | ¹H NMR (400 MHz, CD₃OD) δ 8.18 (d, J = 1.59 Hz, 2H), 7.93 (d, J = 7.8 Hz, 1H), 7.59-7.47 (m, 2H), 7.01 (d, J = 7.8 Hz, 1H), 5.18-5.10 (m, 4H), 4.67 (q, J = 6.7 Hz, 1H), 3.71 (s, 2H), 2.91 (s, 3H), 2.69 (dt, J = 12.4, 6.2 Hz, 1H), 1.54 (d, J = 6.9 Hz, 3H), 1.14 (d, J = 4.0 Hz, 3H), 1.09 (d, J = 4.0 Hz, 3H). |
| | N-{(1ξ)-1-[3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}propan-2-amine | | |
| C80 | 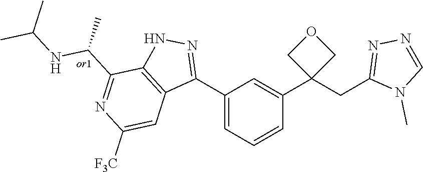 [SMILES: CC(C)N[C@H](C)c1nc(C(F)(F)F)cc2c(-c3cccc(C4(Cc5nncn5C)COC4)c3)n[nH]c12] | 500.4 | ¹H NMR (400 MHz, CD₃OD) δ 8.18 (d, J = 5.9 Hz, 2H), 7.93 (d, J = 8.0 Hz, 1H), 7.58-7.47 (m, 2H), 7.01 (d, J = 8.4 Hz, 1H), 5.19-5.10 (m, 4H), 4.70 (q, J = 6.8 Hz, 1H), 3.71 (s, 2 H), 2.91 (s, 3H), 2.73 (dt, J = 12.6, 6.3 Hz, 1H), 1.55 (d, J = 6.9 Hz, 3H), 1.15 (d, J = 4.0 Hz, 3H), 1.10 (d, J = 4.0 Hz, 3H). |
| | N-{(1ξ)-1-[3-(3-{3-[(4-methyl- | | |
| | 4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}propan-2-amine | | |
| C81 | 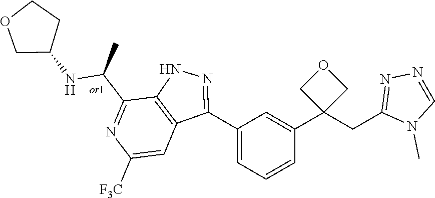 [SMILES: C[C@H](N[C@H]1CCOC1)c1nc(C(F)(F)F)cc2c(-c3cccc(C4(Cc5nncn5C)COC4)c3)n[nH]c12] | 528.3 | ¹H NMR (400 MHz, CD₃OD) δ 8.18 (d, J = 3.2 Hz, 2H), 7.93 (d, J = 7.9 Hz, 1H), 7.54 (t, J = 7.4 Hz, 1H), 7.50 (s, 1H), 7.02 (d, J = 7.8 Hz, 1H), 5.19-5.09 (m, 4H), 4.51 (q, J = 6.8 Hz, 1H), 4.00- 3.93 (m, 1H), 3.79-3.72 (m, 1H), 3.71 (s, 2H), 3.66 (dd, J = 6.1, 8.8 Hz, 1H), 3.51 (dd, J = 4.6, 8.9 Hz, 1H), 3.27 (s, 1H), 2.91 (s, 3H), 2.13-2.04 (m, 1H), 1.93 (qd, J = 6.2, 12.5 Hz, 1H), 1.56 |
| | (3S)-N-{(1ξ)-1-[3-(3-{3-[(4- | | (d, J = 6.8 Hz, 3H). |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}oxolan-3-amine | | |
| C82 | 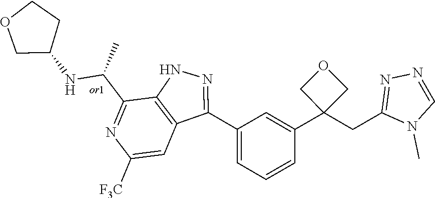 [SMILES: C[C@@H](N[C@H]1CCOC1)c1nc(C(F)(F)F)cc2c(-c3cccc(C4(Cc5nncn5C)COC4)c3)n[nH]c12] | 528.5 | ¹H NMR (400 MHz, CD₃OD) δ 8.18 (d, J = 4.9 Hz, 2H), 7.93 (d, J = 7.8 Hz, 1H), 7.54 (t, J = 7.8 Hz, 1H), 7.50 (s, 1H), 7.02 (d, J = 7.7 Hz, 1H), 5.19-5.09 (m, 4H), 4.47 (q, J = 6.7 Hz, 1H), 3.94- 3.85 (m, 1H), 3.80-3.72 (m, 2H), 3.71 (s, 2H), 3.70-3.64 (m, 1H), 3.30-3.23 (m, 1H), 2.91 (s, 3H), 2.00-1.91 (m, 1H), 1.74-1.62 (m, 1H), 1.55 (d, J = 6.7 Hz, 3H). |
| | (3S)-N-{(1ξ)-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}oxolan-3-amine | | |
| C83 | 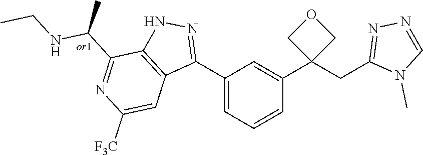 [SMILES: CCN[C@@H](C)c1nc(C(F)(F)F)cc2c(-c3cccc(C4(Cc5nncn5C)COC4)c3)n[nH]c12] | 486.3 | ¹H NMR (400 MHz, CD₃OD) δ 8.24 (s, 1H), 8.15 (s, 1H), 7.91 (br d, J = 7.7 Hz, 1H), 7.53 (t, J = 7.8 Hz, 1H), 7.48 (s, 1H), 7.03 (br d, J = 7.6 Hz, 1H), 5.17-5.08 (m, 4H), 4.80-4.68 (m, 1H), 3.69 (s, 2H), 3.00-2.84 (m, 4H), 2.82-2.69 (m, 1H), 1.64 (d, J = 6.7 Hz, 3H), 1.24 (br t, J = 7.2 Hz, 3H). |
| | (1ξ)-N-ethyl-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethan-1-amine | | |
| C84 | 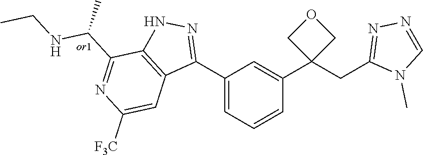 [SMILES: CCN[C@H](C)c1nc(C(F)(F)F)cc2c(-c3cccc(C4(Cc5nncn5C)COC4)c3)n[nH]c12] | 486.5 | ¹H NMR (400 MHz, CD₃OD) δ 8.20 (s, 1H), 8.15 (s, 1H), 7.91 (d, J = 7.7 Hz, 1H), 7.53 (t, J = 7.8 Hz, 1H), 7.48 (s, 1H), 7.01 (br d, J = 7.6 Hz, 1H), 5.12 (s, 4H), 4.63 (q, J = 6.7 Hz, 1H), 3.69 (s, 2H), 2.90 (s, 3H), 2.83- 2.73 (m, 1H), 2.69-2.56 (m, 1H), 1.59 (d, J = 6.7 Hz, 3H), 1.19 (t, J = 7.2 Hz, 3H). |
| | (1ξ)-N-ethyl-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethan-1-amine | | |
| C85 | 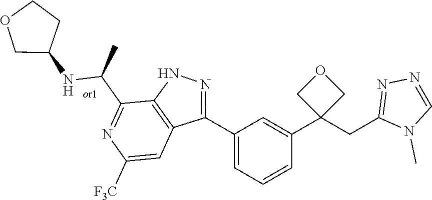 [SMILES: C[C@H](N[C@@H]1CCOC1)c1nc(C(F)(F)F)cc2c(-c3cccc(C4(Cc5nncn5C)COC4)c3)n[nH]c12] | 528.3 | ¹H NMR (400 MHz, DMSO-d₆) δ 8.22 (s, 1H), 8.20 (s, 1H), 7.90 (d, J = 7.8 Hz, 1H), 7.60 (s, 1H), 7.49 (t, J = 7.8 Hz, 1H), 7.09 (d, J = 7.6 Hz, 1H), 5.04-4.89 (m, 4H), 4.41 (br d, J = 6.1 Hz, 1H), 3.78 (q, J = 7.1 Hz, 1H), 3.65- 3.59 (m, 1H), 3.58 (s, 2H), 2.97 (s, 3H), 1.92-1.76 (m, 2H), 1.47 (d, J = 6.6 Hz, 3H). |
| | (3R)-N-{(1ξ)-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}oxolan-3-amine | | |
| C86 | 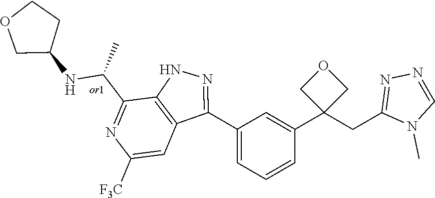 [SMILES: C[C@@H](N[C@@H]1CCOC1)c1nc(C(F)(F)F)cc2c(-c3cccc(C4(Cc5nncn5C)COC4)c3)n[nH]c12] | 528.3 | ¹H NMR (400 MHz, DMSO-d₆ + D₂O) δ 8.17 (s, 1H), 8.14 (s, 1H), 7.87 (d, J = 8.0 Hz, 1H), 7.54- 7.45 (m, 2H), 7.06 (d, J = 7.0 Hz, 1H), 4.99 (d, J = 6.0 Hz, 2H), 4.92 (d, J = 6.3 Hz, 2H), 4.33 (q, J = 6.8 Hz, 1H), 372-3.58 (m, 2H), 3.56 (s, 2H), 3.55-3.46 (m, 2H), 3.13-3.04 (m, 1H), 2.91 (s, 3H), 1.77-1.66 (m, 1H), 1.56- 1.43 (m, 1H), 1.42 (d, J = 6.8 Hz, 3H). |
| | (3R)-N-{(1ξ)-1-[3-(3-{3-[(4- | | |
| | methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}oxolan-3-amine | | |
| C87 | 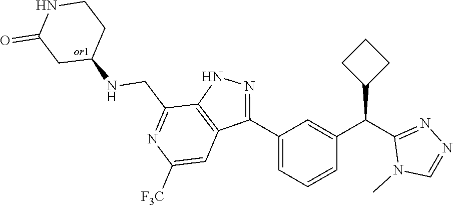 [SMILES: Cn1cnnc1[C@@H](c1cccc(-c2n[nH]c3c(CN[C@@H]4CCNC(=O)C4)nc(C(F)(F)F)cc23)c1)C1CCC1] | 539.4 | ¹H NMR (400 MHz, CD₃OD) δ 8.38 (s, 1H), 8.18 (s, 1H), 7.91- 7.82 (m, 2H), 7.54 (t, J = 7.8 Hz, 1H), 7.35 (d, J = 7.8 Hz, 1H), 4.41 (s, 2H), 4.33 (d, J = 10.8 Hz, 1H), 3.53 (s, 3H), 3.40 (td, J = 5.0, 12.5 Hz, 1H), 3.28-3.11 (m, 2H), 2.69 (dd, J = 4.1, 17.4 Hz, 1H), 2.33-2.23 (m, 2H), 2.20-2.10 (m, 1H), 2.05- 1.67 (m, 6H). |
| | (4ξ)-4-({[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}amino)piperidin-2- | | |
| | one | | |
| C88 | 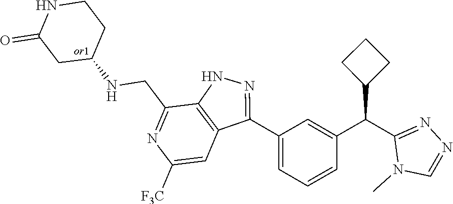 [SMILES: Cn1cnnc1[C@@H](c1cccc(-c2n[nH]c3c(CN[C@H]4CCNC(=O)C4)nc(C(F)(F)F)cc23)c1)C1CCC1] | 539.4 | ¹H NMR (400 MHz, CD₃OD) δ 8.38 (s, 1H), 8.23 (s, 1H), 7.89 (d, J = 7.5 Hz, 1H), 7.84 (s, 1H), 7.55 (t, J = 7.8 Hz, 1H), 7.38 (d, J = 7.8 Hz, 1H), 4.60 (br s, 2H), 4.34 (d, J = 10.8 Hz, 1H), 3.54 (s, 3H), 3.43 (td, J = 4.7, 12.5 Hz, 2H), 2.79 (br dd, J = 5.0, 17.1 Hz, 1H), 2.39 (br dd, J = 9.4, 17.2 Hz, 1H), 2.33-2.17 (m, 2H), 2.07-1.72 (m, 7H). |
| | (4ξ)-4-({[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}amino)piperidin-2- | | |
| | one | | |
| C89 | 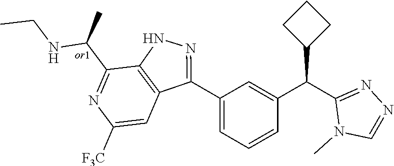 [SMILES: CCN[C@@H](C)c1nc(C(F)(F)F)cc2c(-c3cccc([C@H](c4nncn4C)C4CCC4)c3)n[nH]c12] | 484.1 | ¹H NMR (400 MHz, DMSO-d₆) δ 8.33 (s, 1H), 8.22 (s, 1H), 7.88 (t, J = 2.8 Hz, 1H), 7.33 (d, J = 8.0 Hz, 1H), 4.37-4.30 (m, 2H), 3.44 (s, 3H), 3.34-3.30 (m, 2H), 2.33-2.32 (m, 1H), 2.15-2.05 (m, 1H), 1.84-1.78 (m, 5H), 1.45 (d, J = 6.8 Hz, 3H), 1.00 (t, J = 7.2 Hz, 3H). |
| | (1ξ)-1-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7-yl]-N- | | |
| | ethylethan-1-amine | | |
| C90 | 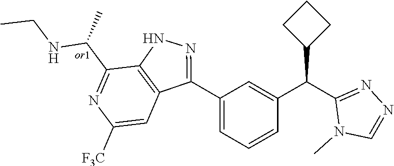 [SMILES: CCN[C@H](C)c1nc(C(F)(F)F)cc2c(-c3cccc([C@H](c4nncn4C)C4CCC4)c3)n[nH]c12] | 484.1 | ¹H NMR (400 MHz, DMSO-d₆) δ 8.33 (s, 1H), 8.22 (s, 1H), 7.88- 7.85 (m, 2H), 7.50 (t, J = 8.0, 1H), 7.33 (d, J = 7.6 Hz, 1H), 4.37-4.32 (m, 2H), 3.44 (s, 3H), 3.24-3.19 (m, 2H), 2.35-2.32 (m, 1H), 2.15-2.05 (m, 1H), 1.84- 1.72 (m, 5H), 1.45 (d, J = 6.8 Hz, 3H), 1.00 (t, J = 7.2 Hz, 3H). |
| | (1ξ)-1-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7-yl]-N- | | |
| | ethylethan-1-amine | | |
| C91 | 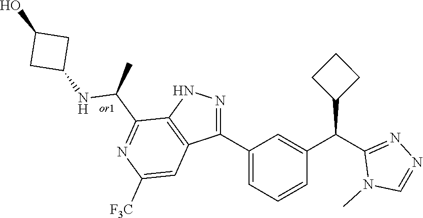 [SMILES: C[C@H](N[C@H]1C[C@H](O)C1)c1nc(C(F)(F)F)cc2c(-c3cccc([C@H](c4nncn4C)C4CCC4)c3)n[nH]c12] | 526.1 | ¹H NMR (400 MHz, DMSO-d₆) δ 8.33 (s, 1H), 8.22 (s, 1H), 7.91- 7.88 (m, 2H), 7.50 (d, J = 3.2 Hz, 1H), 7.33 (d, J = 8.0 Hz, 1H), 4.72 (br s, 1H), 4.37 (d, J = 10.4 Hz, 1H), 4.30-4.16 (m, 2H), 3.44 (s, 3H), 3.22-3.15 (m, 2H), 2.15-2.00 (m, 2H), 1.86-1.72 (m, 8H), 1.43 (d, J = 6.8 Hz, 3H). |
| | (1ξ,3r)-3-({(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)cyclobutan-1-ol | | |
| C92 | 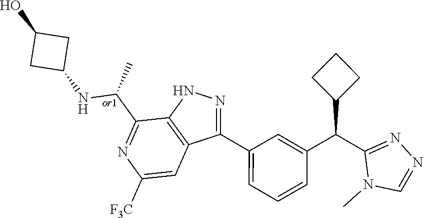 [SMILES: C[C@@H](N[C@H]1C[C@H](O)C1)c1nc(C(F)(F)F)cc2c(-c3cccc([C@H](c4nncn4C)C4CCC4)c3)n[nH]c12] | 526.1 | ¹H NMR (400 MHz, DMSO-d₆) δ 8.33 (s, 1H), 8.22 (s, 1H), 7.89- 7.88 (m, 2H), 7.50 (d, J = 4.8 Hz, 1H), 7.33 (d, J = 8.0 Hz, 1H), 4.73 (br s, 1H), 4.37 (d, J = 10.4 Hz, 1H), 4.30-4.17 (m, 2H), 3.44 (s, 3H), 3.22-3.15 (m, 2H), 2.15-2.00 (m, 2H), 1.86-1.72 (m, 8H), 1.43 (d, J = 6.8 Hz, 3H). |
| | (1ξ,3r)-3-({(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)cyclobutan-1-ol | | |
| C93 | 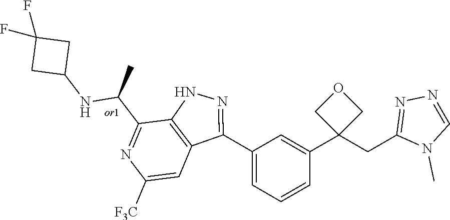 [SMILES: C[C@H](NC1CC(F)(F)C1)c1nc(C(F)(F)F)cc2c(-c3cccc(C4(Cc5nncn5C)COC4)c3)n[nH]c12] | 548.2 | ¹H NMR (400 MHz, DMSO-d₆) δ 8.19 (s, 1H), 8.18 (s, 1H), 7.89 (d, J = 7.6 Hz, 1H), 7.59 (s, 1H), 7.47 (t, J = 7.6 Hz, 1H), 7.06 (d, J = 7.6 Hz, 1H), 5.00-4.93 (m, 4H), 4.38-4.33 (m, 1H), 3.57 (s, 2H), 3.04-3.01 (m, 1H), 2.96 (s, 3H), 2.67-2.66 (m, 1H), 2.44- 2.24 (m, 3H), 1.46 (d, J = 6.8 Hz, 3H). |
| | 3,3-difluoro-N-{(1ξ)-1-[3-(3-{3- | | |
| | [(4-methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}cyclobutan-1-amine | | |
| C94 | 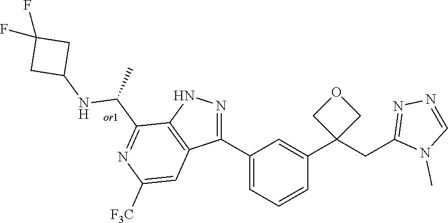 [SMILES: C[C@@H](NC1CC(F)(F)C1)c1nc(C(F)(F)F)cc2c(-c3cccc(C4(Cc5nncn5C)COC4)c3)n[nH]c12] | 548.2 | ¹H NMR (400 MHz, DMSO-d₆) δ 8.18 (s, 2H), 7.87 (d, J = 7.6 Hz, 1H), 7.59 (s, 1H), 7.46 (t, J = 7.6 Hz, 1H), 7.05 (d, J = 7.6 Hz, 1H), 5.00-4.93 (m, 4H), 4.38-4.33 (m, 1H), 3.57 (s, 2H), 3.04-3.01 (m, 1H), 2.96 (s, 3H), 2.68-2.50 (m, 1H), 2.44-2.23 (m, 3H), 1.46 (d, J = 6.8 Hz, 3H). |
| | 3,3-difluoro-N-{(1ξ)-1-[3-(3-{3- | | |
| | [(4-methyl-4H-1,2,4-triazol-3- | | |
| | yl)methyl]oxetan-3-yl}phenyl)- | | |
| | 5-(trifluoromethyl)-1H- | | |
| | pyrazolo[3,4-c]pyridin-7- | | |
| | yl]ethyl}cyclobutan-1-amine | | |
| C95 | 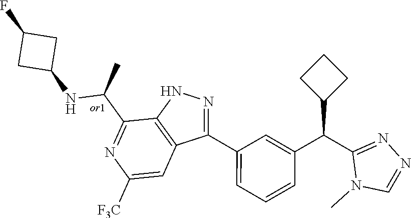 [SMILES: C[C@H](N[C@H]1C[C@@H](F)C1)c1nc(C(F)(F)F)cc2c(-c3cccc([C@H](c4nncn4C)C4CCC4)c3)n[nH]c12] | 528.3 | ¹H NMR (400 MHz, CD₃OD) δ 8.37 (s, 1H), 8.14 (s, 1H), 7.88 (d, J = 8.0 Hz, 1H), 7.83 (s, 1H), 7.53 (t, J = 8.0 Hz, 1H), 7.35 (d, J = 8.0 Hz, 1H), 4.68-4.49 (m, 1H), 4.45-4.40 (m, 1H), 4.35 (d, J = 10.8 Hz, 1H), 3.53 (s, 3H), 2.69-2.66 (m, 1H), 2.27-2.25 (m, 2H), 1.97-1.75 (m, 8H), 1.53 (d, J = 6.8 Hz, 3H). |
| | (1s,3ξ)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-3-fluorocyclobutan-1- | | |
| | amine | | |
| C96 | 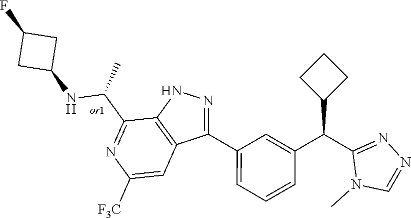 [SMILES: C[C@@H](N[C@H]1C[C@@H](F)C1)c1nc(C(F)(F)F)cc2c(-c3cccc([C@H](c4nncn4C)C4CCC4)c3)n[nH]c12] | 528.3 | ¹H NMR (400 MHz, CD₃OD) δ 8.40 (s, 1H), 8.33 (s, 1H), 7.88 (d, J = 7.6 Hz, 1H), 7.84 (s, 1H), 7.56 (t, J = 7.6 Hz, 1H), 7.40 (d, J = 8.0 Hz, 1H), 5.18-5.13 (m, 1H), 4.79-4.74 (m, 1H), 4.35 (d, J = 10.8 Hz, 1H), 3.54 (s, 3H), 3.47-3.31 (m, 1H), 2.87-2.70 (m, 1H), 2.69-2.67 (m, 1H), 2.60- 2.45 (m, 2H), 2.27-2.15 (m, 1H), 1.96-1.88 (m, 4H), 1.78 (d, J = 6.8 Hz, 3H). |
| | (1s,3ξ)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-3-fluorocyclobutan-1- | | |
| | amine | | |
| C97 | 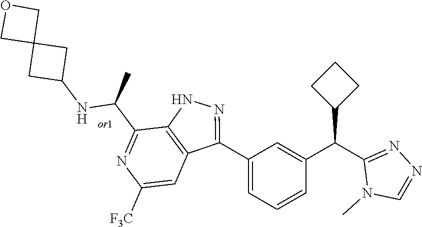 [SMILES: C[C@H](NC1CC2(COC2)C1)c1nc(C(F)(F)F)cc2c(-c3cccc([C@H](c4nncn4C)C4CCC4)c3)n[nH]c12] | 552.3 | ¹H NMR (400 MHz, CD₃OD) δ 8.37 (s, 1H), 8.15 (s, 1H), 7.88 (d, J = 7.6 Hz, 1H), 7.82 (s, 1H), 7.53 (t, J = 7.6 Hz, 1H), 7.34 (d, J = 7.6 Hz), 4.36-4.31 (m, 1H), 3.68 (s, 2H), 3.53-3.48 (m, 5H), 2.73 (s, 2H), 2.33-2.27 (m, 1H), 1.97-1.72 (m, 9H), 1.68 (d, J = 8.8 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-2- | | |
| | oxaspiro[3.3]heptan-6-amine | | |
| C98 | 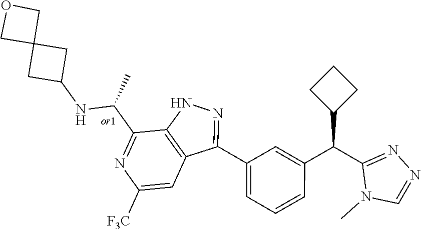 [SMILES: C[C@@H](NC1CC2(COC2)C1)c1nc(C(F)(F)F)cc2c(-c3cccc([C@H](c4nncn4C)C4CCC4)c3)n[nH]c12] | 552.3 | ¹H NMR (400 MHz, CD₃OD) δ 8.37 (s, 1H), 8.15 (s, 1H), 7.88 (d, J = 8.0 Hz, 1H), 7.82 (s, 1H), 7.53 (t, J = 8.0 Hz, 1H), 7.34 (d, J = 8.0 Hz), 4.36-4.31 (m, 1H), 3.68 (s, 2H), 3.52-3.47 (m, 5H), 2.73 (s, 2H), 2.33-2.28 (m, 1H), 1.95-1.68 (m, 9H), 1.59 (d, J = 6.4 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-2- | | |
| | oxaspiro[3.3]heptan-6-amine | | |
| C99 | 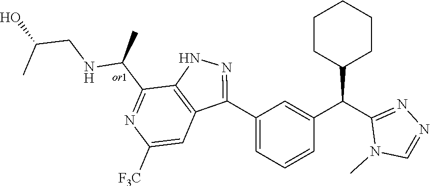 [SMILES: C[C@H](O)CN[C@@H](C)c1nc(C(F)(F)F)cc2c(-c3cccc([C@H](c4nncn4C)C4CCCCC4)c3)n[nH]c12] | 542.2 | ¹H NMR (600 MHz, DMSO-d₆) δ 8.32 (s, 1H), 8.23 (s, 1H), 8.02 (s, 1H), 7.87 (br. d, J = 7.5 Hz, 1H), 7.54-7.47 (m, 1H), 7.46- 7.34 (m, 1H), 4.31 (q, J = 6.6 Hz, 1H), 4.07 (br. d, J = 9.7 Hz, 1H), 3.76-3.68 (m, 1H), 3.53 (s, 3H), 2.56-2.52 (m, 1H), 2.34-2.24 (m, 1H), 2.23-2.13 (m, 1H), 1.76- 1.58 (m, 4H), 1.47 (br. d, J = 6.7 Hz, 3H), 1.34-1.19 (m, 2H), 1.18-1.10 (m, 2H), 1.09-1.01 (m, 1H), 0.99 (br. d, J = 6.2 Hz, 3H), 0.96-0.91 (m, 1H). [α]_{D}²² −32.8° (c 0.1, MeOH) |
| | (2S)-1-({(1ξ)-1-[3-{3-[(R)- | | |
| | cyclohexyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)propan-2-ol | | |
| C100 | 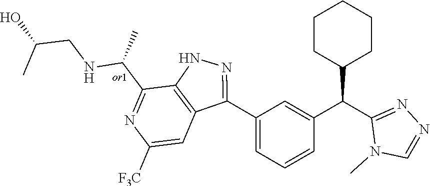 [SMILES: C[C@H](O)CN[C@H](C)c1nc(C(F)(F)F)cc2c(-c3cccc([C@H](c4nncn4C)C4CCCCC4)c3)n[nH]c12] | 542.2 | ¹H NMR (600 MHz, DMSO-d₆) δ 8.32 (s, 1H), 8.23 (s, 1H), 8.02 (s, 1H), 7.87 (br. d, J = 7.5 Hz, 1H), 7.56-7.47 (m, 1H), 7.46- 7.37 (m, 1H), 4.31 (q, J = 6.6 Hz, 1H), 4.07 (br. d, J = 9.7 Hz, 1H), 3.71 (qd, J = 6.1, 11.7 Hz, 1H), 3.53 (s, 3H), 2.49-2.48 (m, 1H), 2.31 (dd, J = 4.4, 11.6 Hz, 1H), 2.29-2.23 (m, 1H), 1.74-1.57 (m, 4H), 1.48 (d, J = 6.7 Hz, 3H), 1.27-1.19 (m, 2H), 1.17-1.10 |
| | (2S)-1-({(1ξ)-1-[3-{3-[(R)- | | (m, 2H), 1.09-1.03 (m, 1H), 1.01 |
| | cyclohexyl(4-methyl-4H-1,2,4- | | (d, J = 6.2 Hz, 3H), 1.00-0.92 |
| | triazol-3-yl)methyl]phenyl}-5- | | (m, 1H). |
| | (trifluoromethyl)-1H- | | [α]_{D}²² −43.1° (c 0.1, MeOH) |
| | pyrazolo[3,4-c]pyridin-7- | | |
| | yl]ethyl}amino)propan-2-ol | | |
| C101 | 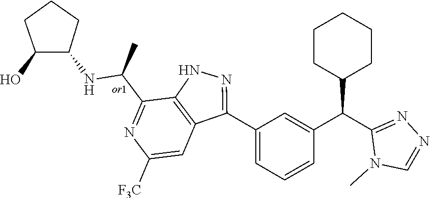 [SMILES: C[C@H](N[C@H]1CCC[C@@H]1O)c1nc(C(F)(F)F)cc2c(-c3cccc([C@H](c4nncn4C)C4CCCCC4)c3)n[nH]c12] | 568.3 | ¹H NMR (600 MHz, DMSO-d₆) δ 8.38-8.28 (m, 1H), 8.24-8.09 (m, 1H), 8.05-7.96 (m, 1H), 7.86 (d, J = 7.7 Hz, 1H), 7.53-7.46 (m, 1H), 7.42 (d, J = 7.7 Hz, 1H), 4.50 (q, J = 6.8 Hz, 1H), 4.07 (d, J = 9.8 Hz, 1H), 3.88-3.82 (m, 1H), 3.53 (s, 3H), 2.70-2.66 (m, 1H), 2.27 (q, J = 10.9 Hz, 1H), 1.85-1.78 (m, 1H), 1.74-1.56 (m, 5H), 1.50-1.44 (m, 3H), 1.42 (d, J = 6.8 Hz, 3H), 1.39-1.31 |
| | (1S,2S)-2-({(1ξ)-1-[3-{3-[(R)- | | (m, 1H), 1.28-1.19 (m, 1H), 1.18- |
| | cyclohexyl(4-methyl-4H-1,2,4- | | 0.88 (m, 5H). |
| | triazol-3-yl)methyl]phenyl}-5- | | [α]_{D}²² −48.2° (c 0.1, MeOH) |
| | (trifluoromethyl)-1H- | | |
| | pyrazolo[3,4-c]pyridin-7- | | |
| | yl]ethyl}amino)cyclopentan-1- | | |
| | ol | | |
| C102 | 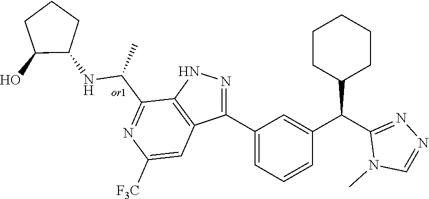 [SMILES: C[C@@H](N[C@H]1CCC[C@@H]1O)c1nc(C(F)(F)F)cc2c(-c3cccc([C@H](c4nncn4C)C4CCCCC4)c3)n[nH]c12] | 568.3 | ¹H NMR (600 MHz, DMSO-d₆) δ 8.32-8.20 (m, 1H), 8.15 (s, 1H), 7.95 (s, 1H), 7.80 (d, J = 7.6 Hz, 1H), 7.46-7.40 (m, 1H), 7.36 (d, J = 7.7 Hz, 1H), 4.33 (q, J = 6.8 Hz, 1H), 4.00 (d, J = 9.8 Hz, 1H), 3.61 (q, J = 6.3 Hz, 2H), 3.46 (s, 3H), 2.48-2.45 (m, 1H), 2.26- 2.16 (m, 1H), 1.79-1.72 (m, 1H), 1.71-1.41 (m, 7H), 1.40-1.37 (m, 3H), 1.34-1.26 (m, 1H), 1.25- 1.14 (m, 2H), 1.11-1.03 (m, |
| | (1S,2S)-2-({(1ξ)-1-[3-{3-[(R)- | | 2H), 1.02-0.95 (m, 1H), 0.93- |
| | cyclohexyl(4-methyl-4H-1,2,4- | | 0.84 (m, 1H). |
| | triazol-3-yl)methyl]phenyl}-5- | | [α]_{D}²² −16.3° (c 0.1, MeOH) |
| | (trifluoromethyl)-1H- | | |
| | pyrazolo[3,4-c]pyridin-7- | | |
| | yl]ethyl}amino)cyclopentan-1- | | |
| | ol | | |
| C103 | 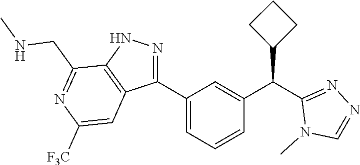 [SMILES: CNCc1nc(C(F)(F)F)cc2c(-c3cccc([C@H](c4nncn4C)C4CCC4)c3)n[nH]c12] | 456.1 | ¹H NMR (600 MHz, DMSO-d₆) δ 8.32 (s, 1H), 8.20 (s, 1H), 7.89 (s, 1H), 7.91-7.86 (m, 1H), 7.47 (s, 1H), 7.30 (s, 1H), 4.34 (br. d, J = 9.9 Hz, 1H), 4.19 (s, 2H), 3.43 (s, 3H), 3.23-3.16 (m, 1H), 2.34 (s, 3H), 2.10 (s, 2H), 1.85- 1.77 (m, 2H), 1.75-1.69 (m, 2H). |
| | 1-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7-yl]-N- | | |
| | methylmethanamine | | |
| C104 | 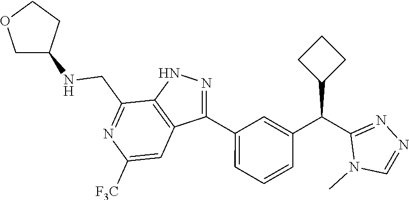 [SMILES: Cn1cnnc1[C@@H](c1cccc(-c2n[nH]c3c(CN[C@@H]4CCOC4)nc(C(F)(F)F)cc23)c1)C1CCC1] | 512.1 | ¹H NMR (600 MHz, DMSO-d₆) δ 8.34 (s, 1H), 8.26 (s, 1H), 7.96- 7.85 (m, 2H), 7.51 (t, J = 7.9 Hz, 1H), 7.35 (d, J = 7.7 Hz, 1H), 4.38 (d, J = 10.5 Hz, 1H), 4.31- 4.13 (m, 2H), 3.78 (q, J = 7.4 Hz, 1H), 3.72-3.62 (m, 2H), 3.52 (br. dd, J = 4.2, 8.7 Hz, 1H), 3.46 (s, 3H), 3.40-3.36 (m, 1H), 3.24- 3.20 (m, 1H), 2.22-2.07 (m, 1H), 2.03-1.90 (m, 1H), 1.90- 1.65 (m, 6H). |
| | (3R)-N-{[3-{3-[(R)-cyclobutyl(4- | | [α]_{D}²² −67.3° (c 0.1, MeOH) |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}oxolan-3-amine | | |
| C105 | 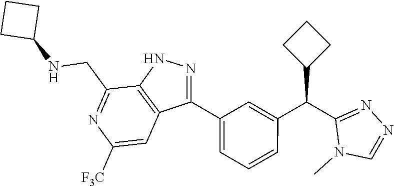 | 496.0 | ¹H NMR (600 MHz, DMSO-d₆) δ 8.32 (s, 1H), 8.20 (s, 1H), 7.93- 7.84 (m, 2H), 7.48 (t, J = 7.6 Hz, 1H), 7.29 (d, J = 7.7 Hz, 1H), 4.35 (d, J = 10.5 Hz, 1H), 4.16 (s, 2H), 3.44 (s, 3H), 3.26-3.22 (m, 1H), 3.21-3.17 (m, 1H), 2.13- 2.06 (m, 1H), 2.00-1.93 (m, 2H), 1.89-1.84 (m, 1H), 1.82-1.76 (m, 3H), 1.74-1.68 (m, 1H), 1.68- 1.60 (m, 2H), 1.59-1.53 (m, 1H), 1.52-1.43 (m, 1H). |
| | N-{[3-{3-[(R)-cyclobutyl(4- | | [α]_{D}²² −58.1° (c 0.1, MeOH) |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}cyclobutanamine | | |
| C106 | 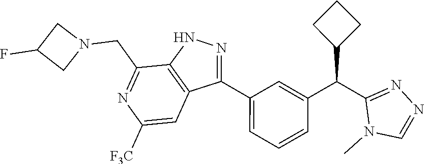 [SMILES: Cn1cnnc1[C@@H](c1cccc(-c2n[nH]c3c(CN4CC(F)C4)nc(C(F)(F)F)cc23)c1)C1CCC1] | 500.0 | ¹H NMR (600 MHz, DMSO-d₆) δ 8.35-8.28 (m, 1H), 8.20 (s, 1H), 7.94-7.83 (m, 2H), 7.47 (t, J = 7.7 Hz, 1H), 7.27 (d, J = 7.7 Hz, 1H), 5.31-5.09 (m, 1H), 4.34 (d, J = 10.5 Hz, 1H), 4.18 (s, 2H), 3.70-3.65 (m, 2H), 3.43 (s, 3H), 3.35-3.32 (m, 1H), 3.31-3.28 (m, 1H), 3.23-3.19 (m, 1H), 2.13- 2.06 (m, 1H), 1.90-1.80 (m, |
| | 3-{3-[(R)-cyclobutyl(4-methyl- | | 2H), 1.80-1.76 (m, 2H), 1.75- |
| | 4H-1,2,4-triazol-3- | | 1.67 (m, 1H). |
| | yl)methyl]phenyl}-7-[(3- | | |
| | fluoroazetidin-1-yl)methyl]-5- | | |
| | (trifluoromethyl)-1H- | | |
| | pyrazolo[3,4-c]pyridine | | |
| C107 | 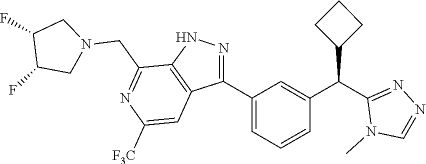 [SMILES: Cn1cnnc1[C@@H](c1cccc(-c2n[nH]c3c(CN4C[C@@H](F)[C@@H](F)C4)nc(C(F)(F)F)cc23)c1)C1CCC1] | 532.0 | ¹H NMR (600 MHz, DMSO-d₆) δ 8.37-8.31 (m, 1H), 8.27 (s, 1H), 7.94-7.82 (m, 2H), 7.50 (t, J = 8.0 Hz, 1H), 7.33 (d, J = 7.7 Hz, 1H), 5.28-5.18 (m, 1H), 5.15- 5.05 (m, 1H), 4.36 (d, J = 10.6 Hz, 1H), 4.23 (s, 2H), 3.44 (s, 3H), 3.20 (td, J = 7.8, 10.1 Hz, 1H), 3.17-3.08 (m, 2H), 2.97- 2.87 (m, 2H), 2.15-2.04 (m, 1H), |
| | 3-{3-[(R)-cyclobutyl(4-methyl- | | 1.90-1.76 (m, 4H), 1.76-1.68 |
| | 4H-1,2,4-triazol-3- | | (m, 1H). |
| | yl)methyl]phenyl}-7-{[(3R,4S)- | | |
| | 3,4-difluoropyrrolidin-1- | | |
| | yl]methyl}-5-(trifluoromethyl)- | | |
| | 1H-pyrazolo[3,4-c]pyridine | | |
| C108 | 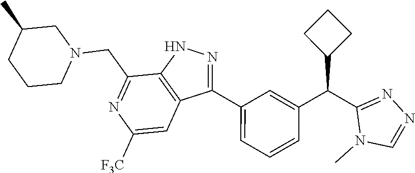 [SMILES: C[C@@H]1CCCN(Cc2nc(C(F)(F)F)cc3c(-c4cccc([C@H](c5nncn5C)C5CCC5)c4)n[nH]c23)C1] | 524.0 | ¹H NMR (600 MHz, DMSO-d₆) δ 8.32 (s, 1H), 8.21 (s, 1H), 7.99- 7.83 (m, 2H), 7.47 (t, J = 7.7 Hz, 1H), 7.28 (d, J = 7.7 Hz, 1H), 4.34 (d, J = 10.5 Hz, 1H), 4.00- 3.93 (m, 2H), 3.43 (s, 3H), 3.24- 3.17 (m, 3H), 2.79 (br. d, J = 7.7 Hz, 2H), 2.15-2.06 (m, 1H), 2.04-1.95 (m, 1H), 1.90-1.80 (m, 2H), 1.73-1.67 (m, 2H), 1.67- 1.58 (m, 2H), 1.53 (br. d, J = |
| | 3-{3-[(R)-cyclobutyl(4-methyl- | | 3.1 Hz, 2H), 0.88-0.79 (m, 1H), |
| | 4H-1,2,4-triazol-3- | | 0.77 (d, J = 6.2 Hz, 3H). |
| | yl)methyl]phenyl}-7-{[(3R)-3- | | |
| | methylpiperidin-1-yl]methyl}-5- | | |
| | (trifluoromethyl)-1H- | | |
| | pyrazolo[3,4-c]pyridine | | |
| C109 | 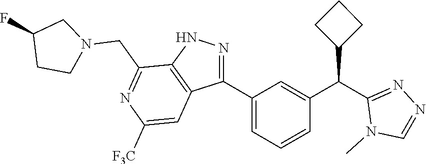 [SMILES: Cn1cnnc1[C@@H](c1cccc(-c2n[nH]c3c(CN4CC[C@@H](F)C4)nc(C(F)(F)F)cc23)c1)C1CCC1] | 514.0 | ¹H NMR (600 MHz, DMSO-d₆) δ 8.32 (s, 1H), 8.25 (s, 1H), 7.95- 7.86 (m, 2H), 7.54-7.44 (m, 1H), 7.31 (d, J = 7.7 Hz, 1H), 5.31- 5.11 (m, 1H), 4.35 (d, J = 10.5 Hz, 1H), 4.25-4.07 (m, 2H), 3.44 (s, 3H), 3.21-3.19 (m, 1H, overlap with H₂O), 2.93-2.78 (m, 3H), 2.59-2.52 (m, 1H), 2.21- 2.06 (m, 2H), 1.95-1.76 (m, 5H), 1.76-1.67 (m, 1H). |
| | 3-{3-[(R)-cyclobutyl(4-methyl- | | |
| | 4H-1,2,4-triazol-3- | | |
| | yl)methyl]phenyl}-7-{[(3R)-3- | | |
| | fluoropyrrolidin-1-yl]methyl}-5- | | |
| | (trifluoromethyl)-1H- | | |
| | pyrazolo[3,4-c]pyridine | | |
| C110 | 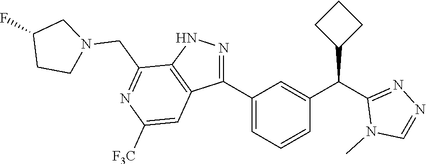 [SMILES: Cn1cnnc1[C@@H](c1cccc(-c2n[nH]c3c(CN4CC[C@H](F)C4)nc(C(F)(F)F)cc23)c1)C1CCC1] | 514.0 | ¹H NMR (600 MHz, DMSO-d₆) δ 8.38-8.29 (m, 1H), 8.26 (s, 1H), 7.94-7.85 (m, 2H), 7.49 (t, J = 8.0 Hz, 1H), 7.32 (d, J = 7.7 Hz, 1H), 5.29-5.12 (m, 1H), 4.36 (d, J = 10.5 Hz, 1H), 4.26-4.09 (m, 2H), 3.44 (s, 3H), 3.22-3.19 (m, 1H), 2.93-2.78 (m, 3H), 2.59- 2.53 (m, 1H), 2.22-2.04 (m, 2H), 1.96-1.76 (m, 5H), 1.76-1.65 |
| | 3-{3-[(R)-cyclobutyl(4-methyl- | | (m, 1H). |
| | 4H-1,2,4-triazol-3- | | |
| | yl)methyl]phenyl}-7-{[(3S)-3- | | |
| | fluoropyrrolidin-1-yl]methyl}-5- | | |
| | (trifluoromethyl)-1H- | | |
| | pyrazolo[3,4-c]pyridine | | |
| C111 | 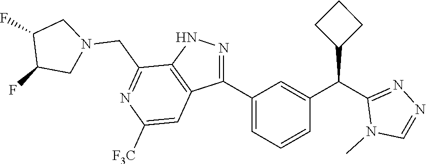 [SMILES: Cn1cnnc1[C@@H](c1cccc(-c2n[nH]c3c(CN4C[C@@H](F)[C@H](F)C4)nc(C(F)(F)F)cc23)c1)C1CCC1] | 532.0 | ¹H NMR (600 MHz, DMSO-d₆) δ 8.34 (s, 1H), 8.29 (s, 1H), 7.92- 7.88 (m, 2H), 7.50 (t, J = 8.0 Hz, 1H), 7.33 (d, J = 7.6 Hz, 1H), 5.32-5.14 (m, 2H), 4.37 (d, J = 10.5 Hz, 1H), 4.33-4.09 (m, 2H), 3.45 (s, 3H), 3.24-3.12 (m, 2H), 2.89-2.78 (m, 2H), 2.10 (dq, J = 10.6, 3.7 Hz, 1H), 1.88- 1.66 (m, 6H). |
| | 3-{3-[(R)-cyclobutyl(4-methyl- | | |
| | 4H-1,2,4-triazol-3- | | |
| | yl)methyl]phenyl}-7-{[(3R,4R)- | | |
| | 3,4-difluoropyrrolidin-1- | | |
| | yl]methyl}-5-(trifluoromethyl)- | | |
| | 1H-pyrazolo[3,4-c]pyridine | | |
| C112 | 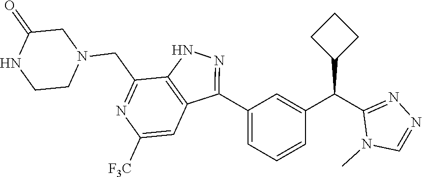 [SMILES: Cn1cnnc1[C@@H](c1cccc(-c2n[nH]c3c(CN4CCNC(=O)C4)nc(C(F)(F)F)cc23)c1)C1CCC1] | 525.0 | ¹H NMR (600 MHz, DMSO-d₆) δ 8.27 (s, 1H), 8.21 (s, 1H), 7.90 (s, 1H), 7.88 (d, J = 7.8 Hz, 1H), 7.49 (t, J = 7.6 Hz, 1H), 7.46 (br. s, 1H), 7.32 (d, J = 7.6 Hz, 1H), 4.34 (d, J = 10.4 Hz, 1H), 4.13 (s, 2H), 3.47 (s, 3H), 3.28-3.21 (m, 1H), 3.21-3.17 (m, 2H), 3.16 (s, 2H), 2.72 (t, J = 5.4 Hz, 2H), 2.17- 2.09 (m, 1H), 1.90-1.85 (m, 2H), 1.88-1.79 (m, 2H), 1.81- |
| | 4-{[3-{3-[(R)-cyclobutyl(4- | | 1.74 (m, 1H). |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methyl}piperazin-2-one | | |
| C113 | 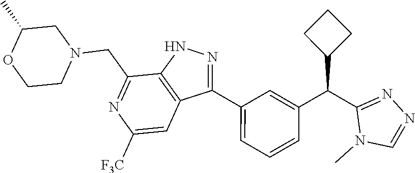 [SMILES: C[C@@H]1CN(Cc2nc(C(F)(F)F)cc3c(-c4cccc([C@H](c5nncn5C)C5CCC5)c4)n[nH]c23)CCO1] | 526.0 | ¹H NMR (600 MHz, DMSO-d₆) δ 8.33 (s, 1H), 8.26 (s, 1H), 7.93- 7.83 (m, 2H), 7.49 (t, J = 7.9 Hz, 1H), 7.32 (d, J = 7.9 Hz, 1H), 4.35 (d, J = 10.5 Hz, 1H), 3.99 (s, 3H), 3.71-3.67 (m, 1H), 3.61- 3.55 (m, 2H), 3.44 (s, 3H), 3.22- 3.19 (m, 1H), 2.75 (br. d, J = 10.8 Hz, 1H), 2.69-2.61 (m, 1H), 2.19 (dt, J = 11.3, 3.3 Hz, 1H), 2.12-2.01 (m, 1H), 1.95-1.86 |
| | 3-{3-[(R)-cyclobutyl(4-methyl- | | (m, 1H), 1.84-1.76 (m, 4H), 1.75- |
| | 4H-1,2,4-triazol-3- | | 1.68 (m, 1H), 0.99 (d, J = 6.4 Hz, |
| | yl)methyl]phenyl}-7-{[(2R)-2- | | 3H). |
| | methylmorpholin-4-yl]methyl}- | | |
| | 5-(trifluoromethyl)-1H- | | |
| | pyrazolo[3,4-c]pyridine | | |
| C114 | 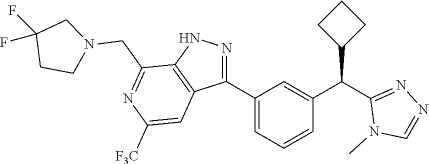 [SMILES: Cn1cnnc1[C@@H](c1cccc(-c2n[nH]c3c(CN4CCC(F)(F)C4)nc(C(F)(F)F)cc23)c1)C1CCC1] | 532.0 | ¹H NMR (600 MHz, DMSO-d₆) δ 8.39-8.30 (m, 1H), 8.23 (s, 1H), 7.93-7.80 (m, 2H), 7.47 (t, J = 7.6 Hz, 1H), 7.28 (d, J = 7.7 Hz, 1H), 4.34 (d, J = 10.5 Hz, 1H), 4.19 (s, 2H), 3.44 (s, 3H), 3.22- 3.18 (m, 1H), 3.04-3.01 (m, 2H), 2.86-2.84 (m, 2H), 2.32-2.20 (m, 2H), 2.14-2.04 (m, 1H), 1.90- 1.81 (m, 2H), 1.80-1.76 (m, 2H), 1.75-1.67 (m, 1H). |
| | 3-{3-[(R)-cyclobutyl(4-methyl- | | |
| | 4H-1,2,4-triazol-3- | | |
| | yl)methyl]phenyl}-7-[(3,3- | | |
| | difluoropyrrolidin-1-yl)methyl]- | | |
| | 5-(trifluoromethyl)-1H- | | |
| | pyrazolo[3,4-c]pyridine | | |
| C115 | 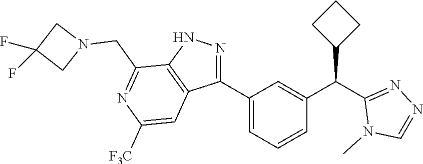 [SMILES: Cn1cnnc1[C@@H](c1cccc(-c2n[nH]c3c(CN4CC(F)(F)C4)nc(C(F)(F)F)cc23)c1)C1CCC1] | 518.0 | ¹H NMR (600 MHz, DMSO-d₆) δ 8.32 (s, 1H), 8.18 (s, 1H), 7.88 (s, 1H), 7.86 (d, J = 7.7 Hz, 1H), 7.45 (t, J = 7.7 Hz, 1H), 7.23 (d, J = 7.7 Hz, 1H), 4.32 (d, J = 10.5 Hz, 1H), 4.28 (s, 2H), 3.79-3.75 (m, 4H), 3.43 (s, 3H), 3.24-3.18 (m, 1H), 2.15-2.07 (m, 1H), 1.90- 1.78 (m, 4H), 1.74-1.67 (m, 1H). |
| | 3-{3-[(R)-cyclobutyl(4-methyl- | | |
| | 4H-1,2,4-triazol-3- | | |
| | yl)methyl]phenyl}-7-[(3,3- | | |
| | difluoroazetidin-1-yl)methyl]-5- | | |
| | (trifluoromethyl)-1H- | | |
| | pyrazolo[3,4-c]pyridine | | |
| C116 | 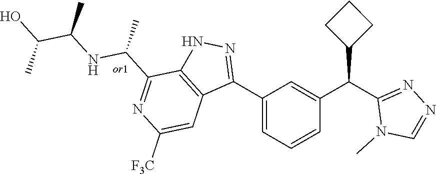 [SMILES: C[C@H](O)[C@@H](C)N[C@H](C)c1nc(C(F)(F)F)cc2c(-c3cccc([C@H](c4nncn4C)C4CCC4)c3)n[nH]c12] | 528.2 | ¹H NMR (600 MHz, DMSO-d₆) δ 8.33 (s, 1H), 8.19 (s, 1H), 7.95- 7.83 (m, 2H), 7.49 (t, J = 8.0 Hz, 1H), 7.31 (d, J = 7.8 Hz, 1H), 4.46 (q, J = 6.7 Hz, 1H), 4.35 (d, J = 10.5 Hz, 1H), 3.61-3.55 (m, 1H), 3.44 (s, 3H), 3.24-3.18 (m, 2H), 2.42-2.28 (m, 1H), 2.17- 2.05 (m, 1H), 1.92-1.84 (m, 1H), 1.80 (dt, J = 2.7, 8.7 Hz, 2H), 1.75-1.65 (m, 1H), 1.49 (d, J = |
| | (2S,3R)-3-({(1ξ)-1-[3-{3-[(R)- | | 6.7 Hz, 3H), 0.99 (t, J = 6.2 Hz, |
| | cyclobutyl(4-methyl-4H-1,2,4- | | 6H). |
| | triazol-3-yl)methyl]phenyl}-5- | | [α]_{D}²² −54.5° (c 0.1, MeOH) |
| | (trifluoromethyl)-1H- | | |
| | pyrazolo[3,4-c]pyridin-7- | | |
| | yl]ethyl}amino)butan-2-ol | | |
| C117 | 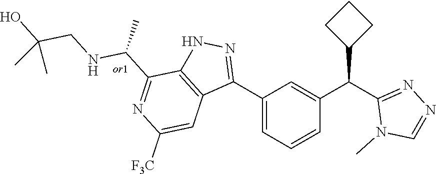 [SMILES: C[C@@H](NCC(C)(C)O)c1nc(C(F)(F)F)cc2c(-c3cccc([C@H](c4nncn4C)C4CCC4)c3)n[nH]c12] | 528.5 | ¹H NMR (400 MHz, DMSO-d₆) δ 8.35 (s, 1H), 8.26 (br s, 1H), 7.93- 7.87 (m, 2H), 7.51 (t, J = 7.9 Hz, 1H), 7.34 (br d, J = 7.5 Hz, 1H), 4.57-4.44 (m, 1H), 4.37 (d, J = 10.6 Hz, 1H), 4.34-4.22 (m, 1H), 3.45 (s, 3H), 3.27-3.05 (m, 1H), 2.29-2.17 (m, 1H), 2.15- 2.05 (m, 1H), 1.92-1.67 (m, 5H), 1.59-1.45 (m, 3H), 1.09 (br s, 6H). |
| | 1-({(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}amino)-2- | | |
| | methylpropan-2-ol | | |
| C118 | 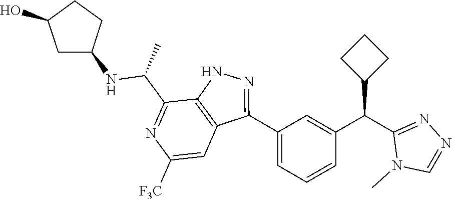 [SMILES: C[C@@H](N[C@@H]1CC[C@H](O)C1)c1nc(C(F)(F)F)cc2c(-c3cccc([C@H](c4nncn4C)C4CCC4)c3)n[nH]c12] | 540.5 | ¹H NMR (600 MHz, CD₃OD) δ 8.36 (s, 1H), 8.13 (s, 1H), 7.87 (td, J = 1.5, 7.8 Hz, 1H), 7.83 (t, J = 1.3 Hz, 1H), 7.52 (t, J = 7.6 Hz, 1H), 7.33 (td, J = 1.1, 7.6 Hz, 1H), 4.50 (q, J = 6.8 Hz, 1H), 4.32 (d, J = 10.9 Hz, 1H), 4.17 (tt, J = 5.3, 4.4 Hz, 1H), 3.53 (s, 3H), 3.36-3.31 (m, 1H), 3.00 (quin, J = 6.4 Hz, 1H), 2.31-2.26 (m, 1H), 2.07 (td, J = 13.3, 6.5 Hz, 1H), 1.97-1.92 (m, 1H), |
| | (1S,3R)-3-({(1R)-1-[3-{3-[(R)- | | 1.99-1.90 (m, 2H), 1.91-1.86 |
| | cyclobutyl(4-methyl-4H-1,2,4- | | (m, 1H), 1.82-1.76 (m, 1H), 1.72- |
| | triazol-3-yl)methyl]phenyl}-5- | | 1.67 (m, 1H), 1.73-1.64 (m, |
| | (trifluoromethyl)-1H- | | 2H), 1.64-1.59 (m, 1H), 1.62- |
| | pyrazolo[3,4-c]pyridin-7- | | 1.56 (m, 1H), 1.52 (d, J = 6.7 Hz, |
| | yl]ethyl}amino)cyclopentanol | | 3H). |
| C119 | 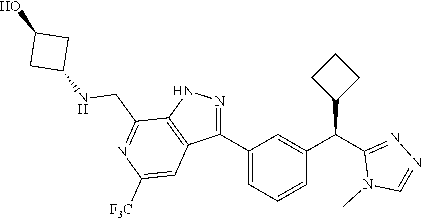 [SMILES: Cn1cnnc1[C@@H](c1cccc(-c2n[nH]c3c(CN[C@H]4C[C@H](O)C4)nc(C(F)(F)F)cc23)c1)C1CCC1] | 512.0 | ¹H NMR (400 MHz, DMSO-d₆) δ 8.27 (s, 1H), 8.16 (s, 1H), 7.83 (s, 1H), 7.82-7.81 (m, 1H), 7.43 (t, J = 8.0 Hz, 1H), 7.26 (br d, J = 7.6 Hz, 1H), 4.73 (br s, 1H), 4.30 (d, J = 10.5 Hz, 1H), 4.21-4.14 (m, 1H), 4.09 (s, 2H), 3.38 (s, 3H), 2.04 (br dd, J = 7.15, 3.12 Hz, 1H), 1.95-1.89 (m, 2H), 1.84-1.66 (m, 8H). [α]_{D}²² −53.3° (c 0.1, MeOH) |
| | (1r,3r)-3-({[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}amino)cyclobutan-1- | | |
| | ol | | |
| C120 | 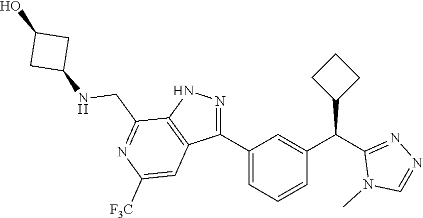 [SMILES: Cn1cnnc1[C@@H](c1cccc(-c2n[nH]c3c(CN[C@H]4C[C@@H](O)C4)nc(C(F)(F)F)cc23)c1)C1CCC1] | 512.1 | ¹H NMR (400 MHz, CD₃OD) δ 8.26 (s, 1H), 8.09 (s, 1H), 7.78 (d, J = 7.8 Hz, 1H), 7.74 (d, J = 1.5 Hz, 1H), 7.44 (t, J = 7.7 Hz, 1H), 7.26 (d, J = 7.7 Hz, 1H), 4.26-4.20 (m, 3H), 3.83 (t, J = 7.0 Hz, 1H), 3.43 (s, 3H), 3.34- 3.23 (m, 1H), 2.96-2.87 (m, 1H), 2.54-2.44 (m, 2H), 2.19 (dt, J = 7.2, 3.9 Hz, 1H), 1.85-1.63 (m, 7H). |
| | (1s,3s)-3-({[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}amino)cyclobutan-1- | | |
| | ol | | |
| C121 | 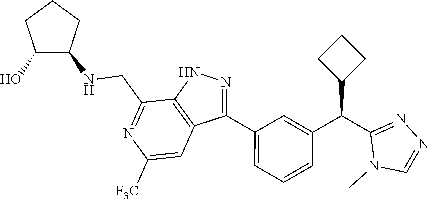 [SMILES: Cn1cnnc1[C@@H](c1cccc(-c2n[nH]c3c(CN[C@@H]4CCC[C@H]4O)nc(C(F)(F)F)cc23)c1)C1CCC1] | 526.0 | |
| | (1R,2R)-2-({[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}amino)cyclopentan- | | |
| | 1-ol | | |
| C122 | 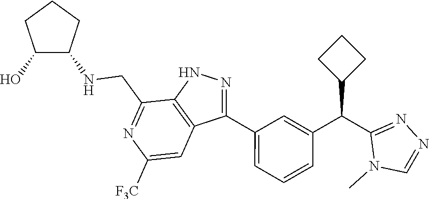 [SMILES: Cn1cnnc1[C@@H](c1cccc(-c2n[nH]c3c(CN[C@H]4CCC[C@H]4O)nc(C(F)(F)F)cc23)c1)C1CCC1] | 526.0 | ¹H NMR (400 MHz, CD₃OD) δ 8.28-8.24 (m, 1H), 8.07-8.00 (m, 1H), 7.89-7.89 (m, 1H), 7.77 (d, J = 7.5 Hz, 1H), 7.74 (s, 1H), 7.58-7.51 (m, 1H), 7.49- 7.38 (m, 2H), 7.27-7.20 (m, 1H), 4.30 (s, 2H), 4.25-4.19 (m, 1H), 4.10-3.98 (m, 1H), 3.43 (s, 3H), 3.38 (dt, J = 3.3, 1.6 Hz, 1H), 2.99-2.88 (m, 1H), 2.26- 2.13 (m, 1H), 1.89-1.81 (m, 4H), 1.76-1.63 (m, 4H), 1.57- 1.41 (m, 3H). |
| | (1R,2S)-2-({[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]methyl}amino)cyclopentan- | | |
| | 1-ol | | |
| C123 | 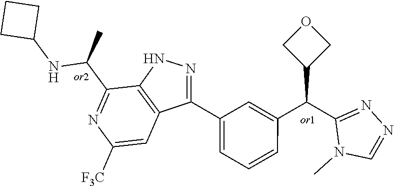 [SMILES: C[C@H](NC1CCC1)c1nc(C(F)(F)F)cc2c(-c3cccc([C@H](c4nncn4C)C4COC4)c3)n[nH]c12] | 512.3 | ¹H NMR (600 MHz, 75° C., DMSO-d₆) δ 8.32 (s, 1H), 8.16 (d, J = 1.4 Hz, 1H), 7.92 (dt, J = 8.0, 1.4 Hz, 1H), 7.88 (d, J = 1.8 Hz, 1H), 7.53 (t, J = 7.7 Hz, 1H), 7.33 (dt, J = 7.7, 1.4 Hz, 1H), 4.87 (d, J = 10.6 Hz, 1H), 4.76 (dd, J = 7.8, 6.2 Hz, 1H), 4.57- 4.53 (m, 2H), 4.39-4.33 (m, 2H), 3.98-3.91 (m, 1H), 3.43 (s, 3H), 3.16-3.10 (m, 1H), 2.08- 2.02 (m, 1H), 1.76-1.67 (m, |
| | N-{(1ξ)-1-[3-{3-[(ξ)-(4-methyl- | | 2H), 1.56-1.48 (m, 2H), 1.47- |
| | 4H-1,2,4-triazol-3-yl)(oxetan-3- | | 1.39 (m, 4H). |
| | yl)methyl]phenyl}-5- | | |
| | (trifluoromethyl)-1H- | | |
| | pyrazolo[3,4-c]pyridin-7- | | |
| | yl]ethyl}cyclobutanamine | | |
| C124 | 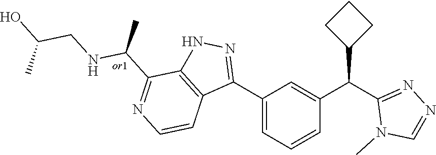 [SMILES: C[C@H](O)CN[C@@H](C)c1nccc2c(-c3cccc([C@H](c4nncn4C)C4CCC4)c3)n[nH]c12] | 446.2 | ¹H NMR (600 MHz, DMSO-d₆) δ 8.33 (br s, 1H), 8.20 (br d, J = 3.6 Hz, 1H), 7.96-7.79 (m, 3H), 7.50-7.44 (m, 1H), 7.29 (br d, J = 6.5 Hz, 1H), 5.47 (br d, J = 9.2 Hz, 1H), 4.69-4.52 (m, 1H), 4.33 (br d, J = 10.3 Hz, 1H), 4.29- 4.23 (m, 1H), 3.83-3.66 (m, 3H), 3.20-3.17 (m, 1H), 2.47- 2.15 (m, 2H), 2.14-2.06 (m, 1H), |
| | (2S)-1-{[(1ξ)-1-(3-{3-[(R)- | | 1.89-1.67 (m, 5H), 1.56-1.39 |
| | cyclobutyl(4-methyl-4H-1,2,4- | | (m, 3H), 1.23 (br s, 1H), 1.06- |
| | triazol-3-yl)methyl]phenyl}-1H- | | 0.83 (m, 3H). |
| | pyrazolo[3,4-c]pyridin-7- | | |
| | yl)ethyl]amino}propan-2-ol | | |
| C125 | 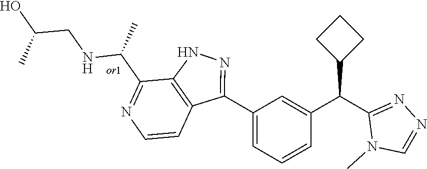 [SMILES: C[C@H](O)CN[C@H](C)c1nccc2c(-c3cccc([C@H](c4nncn4C)C4CCC4)c3)n[nH]c12] | 446.2 | ¹H NMR (600 MHz, DMSO-d₆) δ 8.33 (br s, 1H), 8.20 (br s, 1H), 7.95-7.78 (m, 3H), 7.47 (br s, 1H), 7.29 (br d, J = 6.8 Hz, 1H), 5.77-5.13 (m, 1H), 4.69-4.50 (m, 1H), 4.36-4.30 (m, 1H), 4.29- 4.21 (m, 1H), 3.74-3.63 (m, 3H), 3.25-3.23 (m, 1H), 2.47- 2.15 (m, 2H), 2.14-2.07 (m, 1H), 1.88-1.68 (m, 5H), 1.55-1.40 (m, 3H), 1.25-1.21 (m, 1H), 1.06- |
| | (2S)-1-{[(1ξ)-1-(3-{3-[(R)- | | 0.94 (m, 3H). |
| | cyclobutyl(4-methyl-4H-1,2,4- | | |
| | triazol-3-yl)methyl]phenyl}-1H- | | |
| | pyrazolo[3,4-c]pyridin-7- | | |
| | yl)ethyl]amino}propan-2-ol | | |

### Preparation of Example D1: (3S)—N-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}oxolan-3-amine

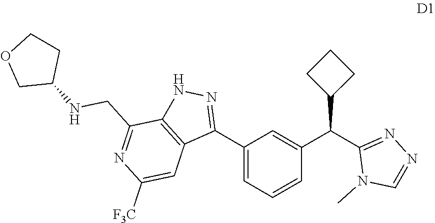 [SMILES: Cn1cnnc1[C@@H](c1cccc(-c2n[nH]c3c(CN[C@H]4CCOC4)nc(C(F)(F)F)cc23)c1)C1CCC1]

Example D1 was prepared according to General Method D.

A mixture of 7-(bromomethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 11) (246 mg, 0.487 mmol), (S)-3-aminotetrahydrofuran (63.6 mg, 0.730 mmol, 0.0638 mL) and DIEA (126 mg, 0.974 mmol, 0.162 mL) in CH₃CN (6.08 mL, c=0.08 M) was stirred at RT for 1 h. The mixture was diluted with brine (10 mL) and extracted with DCM (2×10 mL). The combined organic layers were dried over Na₂SO₄, filtered, and concentrated. The crude residue was purified by flash column chromatography (ISCO, 12 g SiO₂, 0-10% MeOH/1:1 EtOAc/DCM) to give (3S)—N-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}oxolan-3-amine (Example D1) as an off-white solid (159 mg, 64%). The product was further purified with preparative HPLC (Column: ZymorSPHER Pegasus 5 μm 21.2×150 mm Mobile phase A: CO₂ Mobile phase B: MeOH 10-40% B in 4.0 minutes, 120 bar, 70 mL/min) to give the title compound as a white solid (89 gm, 36%). LCMS (ESI+) 512.1 (M+H). ¹H NMR (400 MHz, DMSO-d₆) δ 8.34 (s, 1H), 8.26 (s, 1H), 7.95-7.88 (m, 2H), 7.51 (t, J=8.0 Hz, 1H), 7.35 (d, J=7.7 Hz, 1H), 4.38 (d, J=10.5 Hz, 1H), 4.34-4.17 (m, 2H), 3.79 (q, J=7.4 Hz, 1H), 3.73-3.62 (m, 2H), 3.52 (dd, J=4.2, 8.7 Hz, 1H), 3.46 (s, 3H), 3.41-3.34 (m, 2H), 3.26-3.20 (m, 1H), 2.18-2.06 (m, 1H), 1.96 (qd, J=7.3, 12.4 Hz, 1H), 1.87 (br. s, 6H).

### Example D2: (3R)—N-{(1S)-1-[3-{3-[(R)-cyclobutyl(4-methyl-4H-1,2,4-triazol-3-yl)methyl]-phenyl}-5-(trifluoromethyl)-1H-pyrazolo[3,4-c]pyridin-7-yl]ethyl}oxolan-3-amine; and

### Example D3: (3R)—N-{(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine

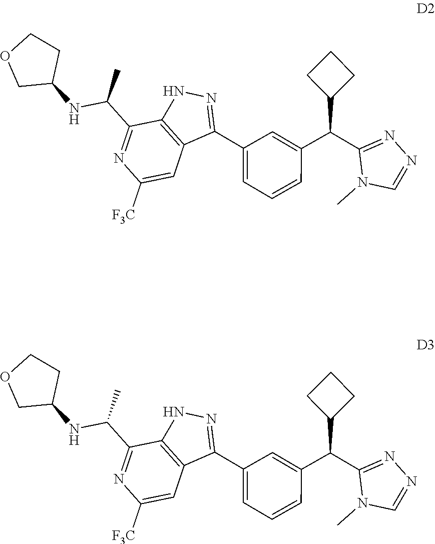 [SMILES: C[C@H](N[C@@H]1CCOC1)c1nc(C(F)(F)F)cc2c(-c3cccc([C@H](c4nncn4C)C4CCC4)c3)n[nH]c12 ; C[C@@H](N[C@@H]1CCOC1)c1nc(C(F)(F)F)cc2c(-c3cccc([C@H](c4nncn4C)C4CCC4)c3)n[nH]c12]

Examples D2 and D3 were prepared according to General Method D.

(3R)-oxolan-3-amine (21.8 mg, 0.306 mmol, 0.0261 mL) was added to a solution of 7-(1-bromoethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 12) (53 mg, 0.10 mmol) in acetonitrile (1 mL, c=0.1M) and stirred at room temperature overnight. The crude mixture was purified by preparative (Column: Phenemonex Gemini NX C18 column, 150×21.2 mm, 5 um column Mobile phase A: Water+10 mM Ammonium Acetate Mobile phase B: Acetonitrile in 9.50 min, 40 mL/min) to give a white solid which was further purified chiral SFC (Column: ES Industries ChromegaChiral CCOF₄, 250×20 mm, 5 um @ 120 bar @ 35° C., 100 mL/min, eluting with 16.0% MeOH+10 mM NH₃ in CO₂ to give (3R)—N-{(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine, the first eluting peak, obtained as a white solid (9.3 mg, 17%). LCMS (APCI+) 526.0 (M+H)⁺. ¹H NMR (DMSO-d₆, 600 MHz) δ 8.27 (s, 1H), 8.17 (s, 1H), 7.88-7.81 (m, 2H), 7.44 (t, 1H, J=7.9 Hz), 7.27 (d, 1H, J=7.8 Hz), 4.30 (d, 2H, J=10.4 Hz), 3.62 (q, 1H, J=7.6 Hz), 3.57-3.51 (m, 1H), 3.49-3.45 (m, 2H), 3.38 (s, 3H), 3.15 (br. s, 1H), 3.07-3.03 (m, 1H), 2.09-1.99 (m, 1H), 1.82-1.61 (m, 6H), 1.49-1.41 (m, 1H), 1.38 (d, 3H, J=6.7 Hz). [α]_{D}²² −38.8° (c 0.1, MeOH) and (3R)—N-{(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}oxolan-3-amine as the second eluting peak, obtained as a white solid (13.5 mg, 25%). LCMS (APCI+) 526.0 (M+H)⁺. ¹H NMR (DMSO-d₆, 600 MHz) δ 8.27 (s, 1H), 8.17 (s, 1H), 7.87-7.78 (m, 2H), 7.44 (t, 1H, J=7.9 Hz), 7.27 (d, 1H, J=7.6 Hz), 4.38-4.32 (m, 1H), 4.31-4.28 (m, 1H), 3.71 (q, 1H, J=7.6 Hz), 3.56-3.50 (m, 1H), 3.42 (br. dd, 1H, J=6.2, 8.4 Hz), 3.38 (s, 3H), 3.17-3.13 (m, 2H), 3.06-3.02 (m, 1H), 2.07-2.00 (m, 1H), 1.85-1.63 (m, 7H), 1.40 (d, 3H, J=6.7 Hz). [α]_{D}²² −76.4° (c 0.1, MeOH).

### Example D18: (2S)-1-({(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol; and

### Example D19: (2S)-1-({(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol

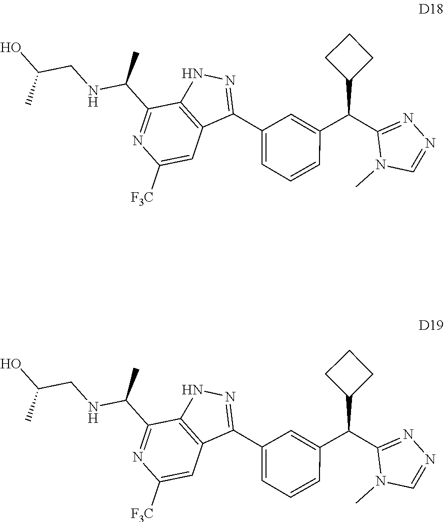 [SMILES: C[C@H](O)CN[C@@H](C)c1nc(C(F)(F)F)cc2c(-c3cccc([C@H](c4nncn4C)C4CCC4)c3)n[nH]c12 ; C[C@H](O)CN[C@@H](C)c1nc(C(F)(F)F)cc2c(-c3cccc([C@H](c4nncn4C)C4CCC4)c3)n[nH]c12]

Examples D18 and D19 were prepared according to General Method D.

(2S)-1-aminopropan-2-ol (0.539 g, 7.18 mmol) and i-Pr₂NEt (0.928 g, 7.18 mmol) were added to a solution of 7-(1-bromoethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 12) (3.73 g, 1.40 mmol) in acetonitrile (20 mL) and stirred at room temperature for 1 h. The mixture was diluted with water (20 mL), EtOAc (50 mL) and adjusted to pH<7 with 1N HCl (10 mL). The organic layer was extracted with water (3×50 mL) and then the aqueous phase was adjusted to pH>7 with aqueous NaHCO₃. The aqueous layer was extracted with EtOAc (3×50 mL). The combined organic layers were dried over Na₂SO₄, filtered, and concentrated. The crude residue was purified by flash column chromatography (ISCO, 20 g SiO₂, 0-15% MeOH/DCM) followed by preparative SFC (Column: ChiralPak IG, 250×30 mm, 10 μm, 80 mL/min, eluting with 40% EtOH+0.1% NH₃ in CO₂). (2S)-1-({(1R)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol (Example D18), the first eluting peak, was obtained as a white solid (154.6 mg, 21%). LCMS (ESI+) 514.3 (M+H)⁺. ¹H NMR (400 MHz, CD₃OD) δ 8.37 (s, 1H), 8.14 (s, 1H), 7.89-7.81 (m, 2H), 7.53 (br t, J=7.6 Hz, 1H), 7.34 (br d, J=7.5 Hz, 1H), 4.43 (q, J=6.5 Hz, 1H), 4.32 (br d, J=10.8 Hz, 1H), 3.94-3.86 (m, 1H), 3.53 (s, 3H), 2.67 (br dd, J=2.8, 11.8 Hz, 1H), 2.38-2.25 (m, 2H), 2.01-1.86 (m, 5H), 1.80 (br dd, J=8.6, 16.6 Hz, 1H), 1.56 (br d, J=6.6 Hz, 3H), 1.10 (br d, J=6.3 Hz, 3H. (2S)-1-({(1S)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol (Example D19), the second eluting peak, was obtained as a white solid (145.0 mg, 20%). LCMS (APCI+) 514.3 (M+H)⁺. ¹H NMR (400 MHz, CD₃OD) δ 8.38 (s, 1H), 8.15 (s, 1H), 7.87 (br d, J=7.8 Hz, 1H), 7.83 (s, 1H), 7.53 (t, J=7.7 Hz, 1H), 7.34 (br d, J=7.6 Hz, 1H), 4.43 (q, J=6.8 Hz, 1H), 4.33 (d, J=10.9 Hz, 1H), 3.86 (ddd, J=4.4, 6.3, 7.4 Hz, 1H), 3.53 (s, 3H), 2.63 (dd, J=7.9, 11.8 Hz, 1H), 2.45 (dd, J=4.2, 11.8 Hz, 1H), 2.33-2.24 (m, 1H), 2.04-1.84 (m, 5H), 1.83-1.76 (m, 1H), 1.56 (d, J=6.8 Hz, 3H), 1.13 (d, J=6.4 Hz, 3H).

Example D32: 3-{3-[(R)-cyclobutyl(4-methyl-4H-1,2,4-triazol-3-yl)methyl]phenyl}-7-{[(2S)-4-methyl-2-(propan-2-yl)piperazin-1-yl]methyl}-5-(trifluoromethyl)-1H-pyrazolo[3,4-c]pyridine

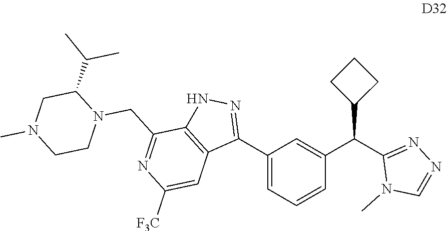 [SMILES: CC(C)[C@H]1CN(C)CCN1Cc1nc(C(F)(F)F)cc2c(-c3cccc([C@H](c4nncn4C)C4CCC4)c3)n[nH]c12]

Example D32 was prepared according to General Method D with N-deprotection and alkylation of the distal amine as outlined below.

### Step 1: tert-butyl (3S)-4-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}-3-(propan-2-yl)piperazine-1-carboxylate

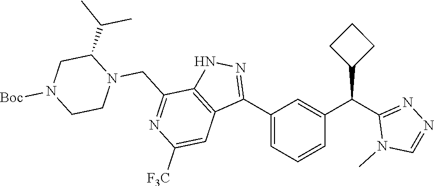 [SMILES: CC(C)[C@H]1CN(C(=O)OC(C)(C)C)CCN1Cc1nc(C(F)(F)F)cc2c(-c3cccc([C@H](c4nncn4C)C4CCC4)c3)n[nH]c12]

To a suspension of 7-(bromomethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 11) (1.3 g, 0.77 mmol) and tert-butyl (3S)-3-(propan-2-yl)piperazine-1-carboxylate (352 mg, 1.54 mmol) in CH₃CN (5 mL) was added i-Pr₂NEt (399 mg, 3.09 mmol). The mixture was stirred at 25° C. for 3 h before it was poured into brine (10 mL) and extracted with EtOAc (2×10 mL). The combined organic layers were washed with brine (10 mL), dried over Na₂SO₄, filtered, and concentrated under reduced pressure. The residue was purified by flash column chromatography (Isco, SiO₂, 0-10% DCM/MeOH) to afford tert-butyl (3S)-4-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}-3-(propan-2-yl)piperazine-1-carboxylate (1000 mg, >99%, 50% purity) as a grey solid. Material was carried forward to the next step without further purification. LCMS (ESI+) 653.3 (M+H)⁺.

### Step 2: 3-{3-[(R)-cyclobutyl(4-methyl-4H-1,2,4-triazol-3-yl)methyl]phenyl}-7-{[(2S)-2-(propan-2-yl)piperazin-1-yl]methyl}-5-(trifluoromethyl)-1H-pyrazolo[3,4-c]pyridine

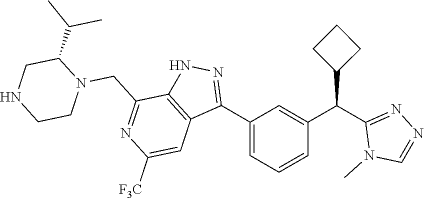 [SMILES: CC(C)[C@H]1CNCCN1Cc1nc(C(F)(F)F)cc2c(-c3cccc([C@H](c4nncn4C)C4CCC4)c3)n[nH]c12]

A solution of tert-butyl (3S)-4-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}-3-(propan-2-yl)piperazine-1-carboxylate (1000 mg, 50% purity, 0.77 mmol) in HCl/dioxane (4 mL) was stirred at 25° C. for 1 h. Then the reaction was concentrated under reduced pressure and DCM (6 mL) was added the reaction was stirred at 25° C. for 30 min. The reaction was filtered to give 3-{3-[(R)-cyclobutyl(4-methyl-4H-1,2,4-triazol-3-yl)methyl]phenyl}-7-{[(2S)-2-(propan-2-yl)piperazin-1-yl]methyl}-5-(trifluoromethyl)-1H-pyrazolo[3,4-c]pyridine (160 mg, 38%) as a yellow solid. Material was carried forward to the next step without further purification. LCMS (ESI+) 553.3 (M+H)⁺. ¹H NMR (400 MHz, CDCl₃) δ 8.53 (br s, 1H), 8.17 (s, 1H), 8.14 (s, 1H), 7.82-7.77 (m, 2H), 7.50 (t, J=7.8 Hz, 1H), 7.33 (br d, J=7.9 Hz, 1H), 4.77 (br d, J=15.8 Hz, 1H), 4.05-3.99 (m, 1H), 3.93 (br d, J=15.2 Hz, 1H), 3.51 (s, 3H), 3.42-3.34 (m, 3H), 3.30-3.11 (m, 3H), 2.92 (br d, J=12.5 Hz, 1H), 2.81-2.72 (m, 2H), 2.54-2.44 (m, 1H), 2.31 (br dd, J=5.7, 8.8 Hz, 1H), 1.98-1.80 (m, 4H), 1.79-1.69 (m, 1H), 1.05 (br t, J=6.1 Hz, 6H).

### Step 3: 3-{3-[(R)-cyclobutyl(4-methyl-4H-1,2,4-triazol-3-yl)methyl]phenyl}-7-{[(2S)-4-methyl-2-(propan-2-yl)piperazin-1-yl]methyl}-5-(trifluoromethyl)-1H-pyrazolo[3,4-c]pyridine

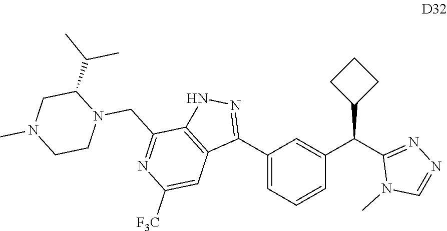 [SMILES: CC(C)[C@H]1CN(C)CCN1Cc1nc(C(F)(F)F)cc2c(-c3cccc([C@H](c4nncn4C)C4CCC4)c3)n[nH]c12]

To a solution of 3-{3-[(R)-cyclobutyl(4-methyl-4H-1,2,4-triazol-3-yl)methyl]phenyl}-7-{[(2S)-2-(propan-2-yl)piperazin-1-yl]methyl}-5-(trifluoromethyl)-1H-pyrazolo[3,4-c]pyridine HCl salt (160 mg, 0.290 mmol) acetic acid (1.74 mg, 0.029 mmol), and formaldehyde solution (37% in water, 94 mg, 1.16 mmol) in EtOH (2 mL) was added NaBH(OAc)₃ (245 mg, 1.16 mmol) and the reaction was stirred at 25° C. for 3 h. After 3 h, the mixture was diluted with water (5 mL) and extracted with EtOAc (3×10 mL). The combined organic layers were washed with brine (10 mL), dried over Na₂SO₄, filtered, and concentrated under reduced pressure. Purification via preparative HPLC (column: Phenomenex C18 (75×30 mm, 3 μm particle size), Flow Rate: 60/0 mL/min, 43-83% CAN in water (ammonium hydroxide)) afforded 3-{3-[(R)-cyclobutyl(4-methyl-4H-1,2,4-triazol-3-yl)methyl]phenyl}-7-{[(2S)-4-methyl-2-(propan-2-yl)piperazin-1-yl]methyl}-5-(trifluoromethyl)-1H-pyrazolo[3,4-c]pyridine (Example D32) (7.1 mg, 4%) as a white solid. LCMS (ESI+) 567.5 (M+H)⁺. ¹H NMR (400 MHz, CDCl₃) δ 8.13 (s, 1H), 8.05 (s, 1H), 7.84-7.79 (m, 2H), 7.50 (t, J=7.6 Hz, 1H), 7.30 (d, J=7.7 Hz, 1H), 4.77 (d, J=15.8 Hz, 1H), 4.01 (d, J=10.6 Hz, 1H), 3.77 (d, J=15.8 Hz, 1H), 3.47-3.38 (m, 4H), 2.93 (br d, J=11.4 Hz, 1H), 2.79-2.71 (m, 2H), 2.58-2.51 (m, 2H), 2.41-2.32 (m, 5H), 2.27-2.20 (m, 1H), 2.16-2.09 (m, 1H), 2.00-1.81 (m, 5H), 1.05 (dd, J=7.0, 16.5 Hz, 6H), 0.93-0.78 (m, 1H).

Examples D4-D17, D20-D31 and D34-D98 reported in Table 4 were synthesized with non-critical changes or substitutions to the exemplified procedures for Examples D1, D2 and D3 that one skilled in the art would be able to realize.

TABLE 4

| | | LCMS | |
|---|---|---|---|
| Example | | (m/z) | |
| Number | Structure and Name | (M + H)⁺ | ¹H NMR and Rotation |
| D4 | 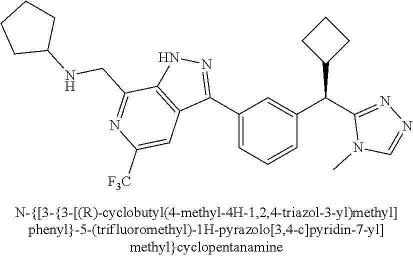 [SMILES: Cn1cnnc1[C@@H](c1cccc(-c2n[nH]c3c(CNC4CCCC4)nc(C(F)(F)F)cc23)c1)C1CCC1] | 510.0 | ¹H NMR (400 MHz, DMSO- d₆) δ 8.41-8.27 (m, 1H), 8.22- 8.14 (m, 1H), 7.96-7.85 (m, 2H), 7.49 (t, J = 7.6 Hz, 1H), 7.30 (d, J = 7.7 Hz, 1H), 4.36 (d, J = 10.5 Hz, 1H), 4.24 (s, 2H), 3.45 (s, 3H), 3.23-3.21 (m, 1H), 3.07 (td, J = 6.2, 12.3 Hz, 2H), 2.16-2.08 (m, 1H), 1.87-1.78 (m, 4H), 1.75-1.67 (m, 3H), 1.66-1.58 (m, 2H), 1.49-1.35 (m, 4H) |
| D5 | 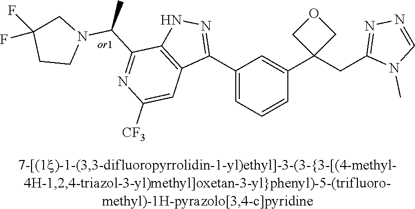 [SMILES: C[C@@H](c1nc(C(F)(F)F)cc2c(-c3cccc(C4(Cc5nncn5C)COC4)c3)n[nH]c12)N1CCC(F)(F)C1] | 548.3 | ¹H NMR (400 MHz, CD₃OD) δ 8.21 (s, 1H), 8.19 (s, 1H), 7.93 (d, J = 7.8 Hz, 1H), 7.55 (t, J = 7.7 Hz, 1H), 7.50 (s, 1H), 7.02 (d, J = 7.8 Hz, 1H), 5.17-5.12 (m, 4H), 4.07 (q, J = 6.4 Hz, 1H), 3.71 (s, 2H), 3.18-3.01 (m, 2H), 2.90 (s, 4H), 2.75-2.63 (m, 1H), 2.48- 2.20 (m, 2H), 1.59 (d, J = 6.8 Hz, 3H) |
| D6 | 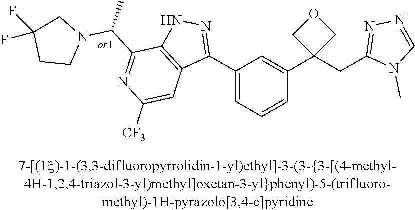 [SMILES: C[C@H](c1nc(C(F)(F)F)cc2c(-c3cccc(C4(Cc5nncn5C)COC4)c3)n[nH]c12)N1CCC(F)(F)C1] | 548.4 | ¹H NMR (400 MHz, CD₃OD) δ 8.21 (s, 1H), 8.19 (s, 1H), 7.93 (d, J = 7.5 Hz, 1H), 7.55 (t, J = 7.8 Hz, 1H), 7.50 (s, 1H), 7.01 (d, J = 8.0 Hz, 1H), 5.18-5.09 (m, 4H), 4.07 (q, J = 6.8 Hz, 1H), 3.71 (s, 2H), 3.20-3.02 (m, 2H), 2.90 (s, 4H), 2.68 (q, J = 7.5 Hz, 1H), 2.46-2.24 (m, 2H), 1.59 (d, J = 6.8 Hz, 3H) |
| D7 | 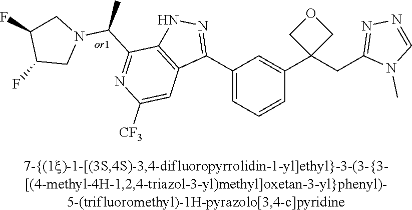 [SMILES: C[C@@H](c1nc(C(F)(F)F)cc2c(-c3cccc(C4(Cc5nncn5C)COC4)c3)n[nH]c12)N1C[C@H](F)[C@@H](F)C1] | 548.3 | ¹H NMR (400 MHz, CD₃OD) δ 8.20 (s, 1H), 8.19 (s, 1H), 7.93 (d, J = 7.8 Hz, 1H), 7.54 (t, J = 7.8 Hz, 1H), 7.51 (s, 1H), 7.00 (d, J = 7.8 Hz, 1H), 5.28 (br d, J = 16.3 Hz, 1H), 5.19-5.10 (m, 5H), 4.08 (q, J = 6.5 Hz, 1H), 3.71 (s, 2H), 3.36 (br s, 1H), 3.28 (br dd, J = 5.6, 11.2 Hz, 1H), 2.90 (s, 3H), 2.77-2.62 (m, 2H), 1.60 (d, J = 6.8 Hz, 3H) |
| D8 | 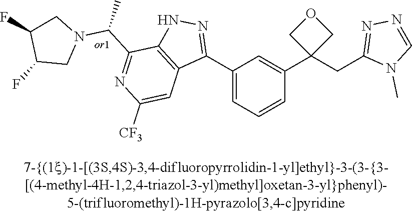 [SMILES: C[C@H](c1nc(C(F)(F)F)cc2c(-c3cccc(C4(Cc5nncn5C)COC4)c3)n[nH]c12)N1C[C@H](F)[C@@H](F)C1] | 548.4 | ¹H NMR (400 MHz, CD₃OD) δ 8.21 (s, 1H), 8.19 (s, 1H), 7.93 (d, J = 7.8 Hz, 1H), 7.55 (t, J = 7.8 Hz, 1H), 7.50 (s, 1H), 7.02 (br d, J = 7.5 Hz, 1H), 5.26 (br d, J = 16.3 Hz, 1H), 5.18-5.10 (m, 5H), 4.27 (br s, 1H), 3.71 (s, 2H), 3.21- 2.98 (m, 4H), 2.91 (s, 3H), 1.63 (d, J = 6.5 Hz, 3H) |
| D9 | 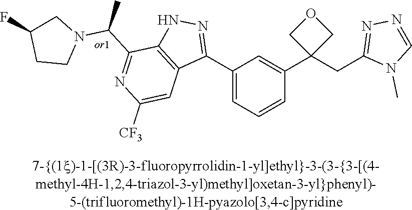 [SMILES: C[C@@H](c1nc(C(F)(F)F)cc2c(-c3cccc(C4(Cc5nncn5C)COC4)c3)n[nH]c12)N1CC[C@@H](F)C1] | 530.4 | ¹H NMR (400 MHz, CD₃OD) δ 8.20 (s, 1H), 8.18 (s, 1H), 7.93 (d, J = 7.6 Hz, 1H), 7.55 (t, J = 7.6 Hz, 1H), 7.50 (s, 1H), 7.02 (br d, J = 7.7 Hz, 1H), 5.33-5.10 (m, 5H), 4.11 (br s, 1H), 3.71 (s, 2H), 3.14- 3.01 (m, 1H), 2.91 (s, 3H), 2.80 (m, 3H), 2.32-2.16 (m, 1H), 2.15-1.99 (m, 1H), 1.62 (d, J = 6.7 Hz, 3H) |
| D10 | 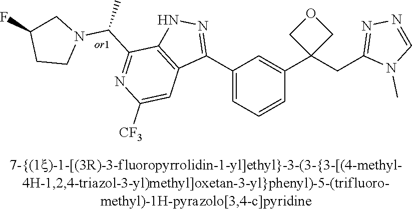 [SMILES: C[C@H](c1nc(C(F)(F)F)cc2c(-c3cccc(C4(Cc5nncn5C)COC4)c3)n[nH]c12)N1CC[C@@H](F)C1] | 530.4 | ¹H NMR (400 MHz, CD₃OD) δ 8.20 (s, 1H), 8.18 (s, 1H), 7.93 (d, J = 7.6 Hz, 1H), 7.55 (t, J = 7.6 Hz, 1H), 7.50 (s, 1H), 7.01 (br d, J = 7.4 Hz, 1H), 5.33-5.11 (m, 5H), 4.06 (br s, 1H), 3.71 (s, 2H), 3.14 (br d, J = 7.2 Hz, 1H), 3.05- 2.70 (m, 5H), 2.48 (br s, 1H), 2.37-2.00 (m, 2H), 1.61 (d, J = 6.7 Hz, 3H) |
| D11 | 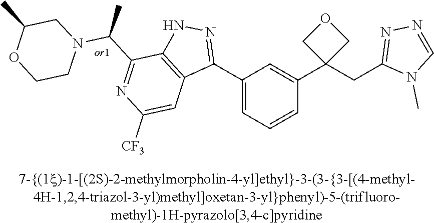 [SMILES: C[C@H]1CN([C@@H](C)c2nc(C(F)(F)F)cc3c(-c4cccc(C5(Cc6nncn6C)COC5)c4)n[nH]c23)CCO1] | 542.4 | ¹H NMR (400 MHz, CD₃OD) δ 8.17 (s, 2H), 7.91 (d, J = 7.7 Hz, 1H), 7.53 (t, J = 7.6 Hz, 1H), 7.48 (s, 1H), 7.00 (br d, J = 8.1 Hz, 1H), 5.20-5.09 (m, 4H), 4.06 (q, J = 6.6 Hz, 1H), 3.82-3.62 (m, 5H), 2.99 (br d, J = 11.4 Hz, 1H), 2.89 (s, 3H), 2.53-2.45 (m, 1H), 2.42- 2.33 (m, 1H), 1.95 (t, J = 10.6 Hz, 1H), 1.54 (d, J = 6.7 Hz, 3H), 1.14 (d, J = 6.2 Hz, 3H) |
| D12 | 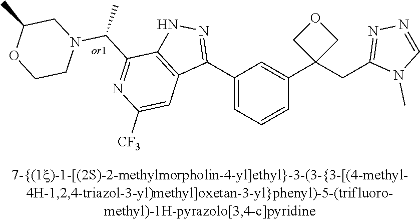 [SMILES: C[C@H](c1nc(C(F)(F)F)cc2c(-c3cccc(C4(Cc5nncn5C)COC4)c3)n[nH]c12)N1CCO[C@@H](C)C1] | 542.4 | ¹H NMR (400 MHz, CD₃OD) δ 8.16 (s, 2H), 7.90 (d, J = 7.7 Hz, 1H), 7.52 (t, J = 7.7 Hz, 1H), 7.47 (s, 1H), 7.00 (d, J = 7.7 Hz, 1H), 5.20-5.05 (m, 4H), 4.04 (q, J = 6.4 Hz, 1H), 3.93-3.76 (m, 2H), 3.73-3.59 (m, 3H), 2.93 (br d, J = 11.2 Hz, 1H), 2.88 (s, 3H), 2.55 (br d, J = 11.4 Hz, 1H), 2.27 (dt, J = 3.4, 11.2 Hz, 1H), 2.11- 1.97 (m, 1H), 1.54 (d, J = 6.8 Hz, 3H), 1.01 (d, J = 6.2 Hz, 3H) |
| D13 | 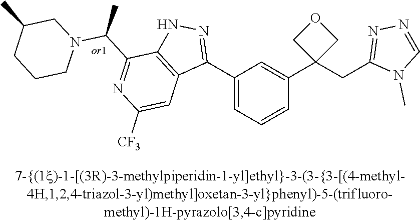 [SMILES: C[C@@H]1CCCN([C@@H](C)c2nc(C(F)(F)F)cc3c(-c4cccc(C5(Cc6nncn6C)COC5)c4)n[nH]c23)C1] | 540.3 | ¹H NMR (400 MHz, CD₃OD) δ 8.16 (s, 1H), 8.15 (s, 1H), 7.91 (d, J = 7.9 Hz, 1H), 7.52 (t, J = 7.7 Hz, 1H), 7.48 (s, 1H), 6.99 (d, J = 8.4 Hz, 1H), 5.21-5.06 (m, 4H), 4.14 (q, J = 6.8 Hz, 1H), 3.77-3.63 (m, 2H), 2.99 (br d, J = 8.1 Hz, 1H), 2.89 (s, 3H), 2.76 (br d, J = 10.6 Hz, 1H), 2.22 (br dd, J = 11.1, 14.6 Hz, 1H), 1.82- 1.68 (m, 3H), 1.63 (br d, J = 3.4 Hz, 2H), 1.55 (d, J = 6.8 Hz, 3H), 0.93-0.86 (m, 1H), |
| | | | 0.87 (d, J = 6.5 Hz, 3H) |
| D14 | 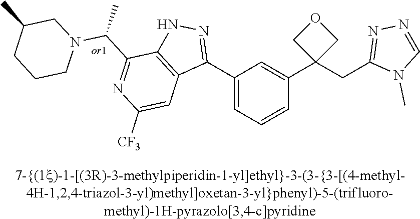 [SMILES: C[C@@H]1CCCN([C@H](C)c2nc(C(F)(F)F)cc3c(-c4cccc(C5(Cc6nncn6C)COC5)c4)n[nH]c23)C1] | 540.5 | ¹H NMR (400 MHz, CD₃OD) δ 8.16 (s, 1H), 8.15 (s, 1H), 7.91 (d, J = 7.8 Hz, 1H), 7.52 (t, J = 7.7 Hz, 1H), 7.48 (s, 1H), 6.99 (d, J = 8.4 Hz, 1H), 5.19-5.08 (m, 4H), 4.23-4.08 (m, 1H), 3.69 (s, 2H), 3.05 (br d, J = 10.6 Hz, 1H), 2.89 (s, 3H), 2.80-2.66 (m, 1H), 2.11- 1.97 (m, 1H), 1.88 (br t, J = 10.9 Hz, 1H), 1.79-1.62 (m, 4H), 1.54 (d, J = 6.7 Hz, 3H), 0.97-0.83 (m, 1H), 0.79 (d, J = 6.5 Hz, 3H) |
| D15 | 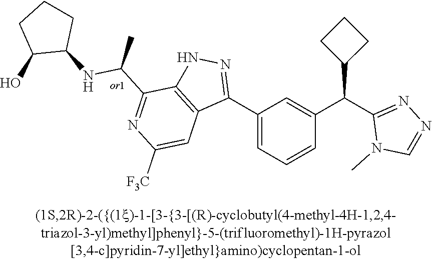 [SMILES: C[C@H](N[C@@H]1CCC[C@@H]1O)c1nc(C(F)(F)F)cc2c(-c3cccc([C@H](c4nncn4C)C4CCC4)c3)n[nH]c12] | 540.2 | ¹H NMR (400 MHz, CD₃OD) δ 8.26 (s, 1H), 8.09 (s, 1H), 7.80-7.76 (m, 1H), 7.73 (t, J = 1.5 Hz, 1H), 7.44 (t, J = 7.7 Hz, 1H), 7.31-7.23 (m, 1H), 4.72-4.65 (m, 1H), 4.23 (d, J = 10.9 Hz, 1H), 4.09 (dt, J = 2.4, 4.2 Hz, 1H), 3.44 (s, 3H), 3.31-3.23 (m, 1H), 2.93 (ddd, J = 4.4, 7.5, 9.7 Hz, 1H), 2.26-2.11 (m, 1H), 1.91-1.85 (m, 2H), 1.83-1.76 (m, 2H), 1.75-1.59 (m, 5H), 1.59-1.45 (m, 4H), 1.45-1.28 (m, 1H) [a]_{D}²² −23.3° (c 0.1, MeOH) |
| D16 | 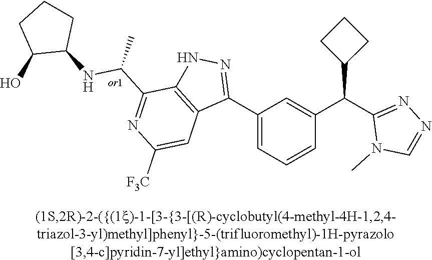 [SMILES: C[C@@H](N[C@@H]1CCC[C@@H]1O)c1nc(C(F)(F)F)cc2c(-c3cccc([C@H](c4nncn4C)C4CCC4)c3)n[nH]c12] | 540.2 | ¹H NMR (400 MHz, CD₃OD) δ = 8.26 (s, 1H), 8.06 (s, 1H), 7.83-7.75 (m, 1H), 7.74-7.68 (m, 1H), 7.43 (t, J = 7.7 Hz, 1H), 7.25 (d, J = 7.8 Hz, 1H), 4.51 (q, J = 6.7 Hz, 1H), 4.22 (d, J = 10.9 Hz, 1H), 3.83 (dt, J = 2.7, 4.6 Hz, 1H), 3.43 (s, 3H), 3.29-3.23 (m, 1H), 2.83 (ddd, J = 4.6, 7.4, 9.1 Hz, 1H), 2.26-2.13 (m, 1H), 1.95- 1.83 (m, 4H), 1.81-1.62 (m, 4H), 1.61-1.52 (m, 2H), 1.51 (d, J = 6.7 Hz, 3H), 1.47-1.32 (m, 1H) [α]_{D}²² −70.7° (c 0.1, MeOH) |
| D17 | 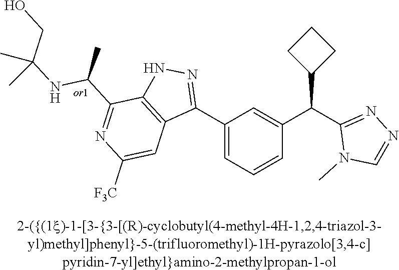 [SMILES: C[C@H](NC(C)(C)CO)c1nc(C(F)(F)F)cc2c(-c3cccc([C@H](c4nncn4C)C4CCC4)c3)n[nH]c12] | 528.3 | ¹H NMR (400 MHz, CD₃OD) δ 8.37 (s, 1H), 8.11 (s, 1H), 7.87 (d, J = 7.9 Hz, 1H), 7.83 (s, 1H), 7.53 (t, J = 7.7 Hz, 1H), 7.34 (d, J = 7.5 Hz, 1H), 4.68-4.56 (m, 3H), 4.33 (d, J = 11.0 Hz, 1H), 3.53 (s, 3H), 3.45 (d, J = 10.8 Hz, 1H), 3.29-3.23 (m, 1H), 2.35-2.23 (m, 1H), 2.02-1.86 (m, 4H), 1.85-1.73 (m, 1H), 1.53 (d, J = 6.8 Hz, 3H), 1.01 (s, 3H), 0.89 (s, 3H) |
| D20 | 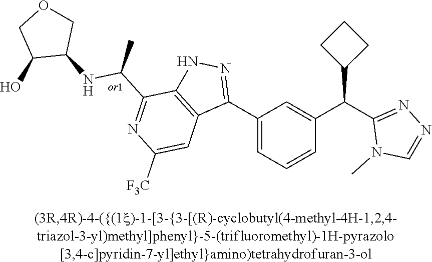 [SMILES: C[C@H](N[C@@H]1COC[C@@H]1O)c1nc(C(F)(F)F)cc2c(-c3cccc([C@H](c4nncn4C)C4CCC4)c3)n[nH]c12] | 542.4 | ¹H NMR (400 MHz, CD₃OD) δ 8.37 (s, 1H), 8.15 (s, 1H), 7.87 (d, J = 7.8 Hz, 1H), 7.83 (s, 1H), 7.54 (t, J = 7.7 Hz, 1H), 7.34 (d, J = 7.8 Hz, 1H), 4.50 (q, J = 6.8 Hz, 1H), 4.33 (d, J = 10.9 Hz, 1H), 4.28 (t, J = 3.7 Hz, 1H), 3.87-3.82 (m, 1H), 3.79-3.74 (m, 1H), 3.68 (t, J = 8.0 Hz, 1H), 3.53 (s, 3H), 3.46 (t, J = 8.8 Hz, 1H), 3.14-3.06 (m, 1H), 2.36-2.23 (m, 1H), 2.04-1.85 (m, 5H), 1.85-1.73 (m, 1H), 1.57 (d, J = 6.8 Hz, 3H) |
| D21 | 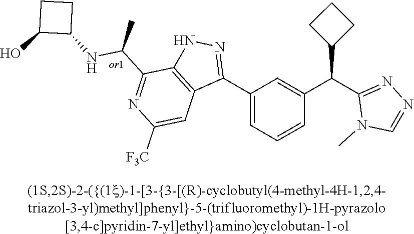 [SMILES: C[C@H](N[C@H]1CC[C@@H]1O)c1nc(C(F)(F)F)cc2c(-c3cccc([C@H](c4nncn4C)C4CCC4)c3)n[nH]c12] | 526.2 | ¹H NMR (400 MHz, CD₃OD) δ 8.36 (s, 1H), 8.14 (s, 1H), 7.87 (br d, J = 7.9 Hz, 1H), 7.83 (s, 1H), 7.53 (t, J = 7.7 Hz, 1H), 7.35 (br d, J = 7.6 Hz, 1H), 4.54 (q, J = 6.8 Hz, 1H), 4.40-4.25 (m, 1H), 3.80 (q, J = 7.5 Hz, 1H), 3.53 (s, 3H), 3.07-2.86 (m, 1H), 2.39- 2.23 (m, 1H), 2.07-1.90 (m, 7H), 1.85-1.72 (m, 1H), 1.54 (d, J = 6.8 Hz, 3H), 1.38-1.25 (m, 2H) [α]_{D}²² −11.2° (c 0.1, MeOH) |
| D22 | 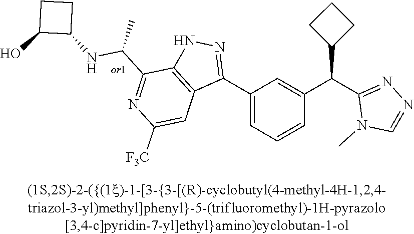 [SMILES: C[C@@H](N[C@H]1CC[C@@H]1O)c1nc(C(F)(F)F)cc2c(-c3cccc([C@H](c4nncn4C)C4CCC4)c3)n[nH]c12] | 526.2 | ¹H NMR (400 MHz, CD₃OD) δ 8.36 (s, 1H), 8.14 (s, 1H), 7.87 (d, J = 7.8 Hz, 1H), 7.83 (s, 1H), 7.53 (t, J = 7.7 Hz, 1H), 7.35 (d, J = 7.8 Hz, 1H), 4.62 (q, J = 6.8 Hz, 1H), 4.33 (d, J = 10.9 Hz, 1H), 3.90 (q, J = 7.7 Hz, 1H), 3.53 (s, 3H), 2.99 (q, J = 8.2 Hz, 1H), 2.35- 2.22 (m, 1H), 2.03-1.93 (m, 4H), 1.85-1.71 (m, 1H), 1.63- 1.50 (m, 4H), 1.42-1.31 (m, 2H), 1.17-1.03 (m, 1H), 0.98- 0.83 (m, 1H) [α]_{D}²² −50.6° (c 0.1, MeOH) |
| D23 | 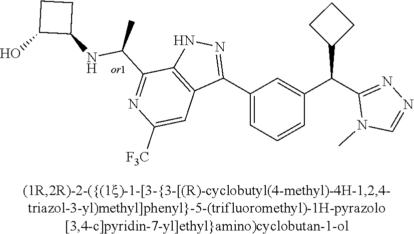 [SMILES: C[C@H](N[C@@H]1CC[C@H]1O)c1nc(C(F)(F)F)cc2c(-c3cccc([C@H](c4nncn4C)C4CCC4)c3)n[nH]c12] | 526.1 | ¹H NMR (400 MHz, CD₃OD) δ 8.39 (s, 1H), 8.16 (s, 1H), 7.89 (d, J = 7.4 Hz, 1H), 7.85 (s, 1H), 7.55 (t, J = 7.7 Hz, 1H), 7.36 (d, J = 7.7 Hz, 1H), 4.62 (q, J = 6.7 Hz, 2H), 4.35 (d, J = 10.9 Hz, 1H), 3.91 (q, J = 7.6 Hz, 1H), 3.55 (s, 3H), 2.99 (q, J = 7.9 Hz, 1H), 2.35- 2.26 (m, 1H), 2.03-1.87 (m, 6H), 1.87-1.76 (m, 1H), 1.56 (d, J = 6.7 Hz, 4H), 1.41-1.29 (m, 1H), 1.18-1.04 (m, 1H) |
| D24 | 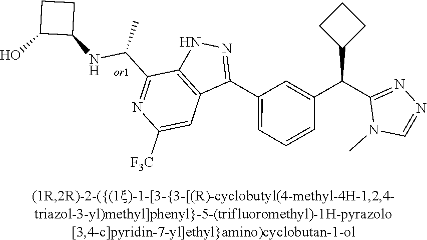 [SMILES: C[C@@H](N[C@@H]1CC[C@H]1O)c1nc(C(F)(F)F)cc2c(-c3cccc([C@H](c4nncn4C)C4CCC4)c3)n[nH]c12] | 526.1 | ¹H NMR (400 MHz, CD₃OD) δ 8.35 (s, 1H), 8.22 (s, 1H), 8.22 (s, 1H), 7.92-7.87 (m, 2H), 7.51 (t, J = 7.9 Hz, 1H), 7.34 (d, J = 7.7 Hz, 1H), 4.84 (br s, 1H), 4.41-4.33 (m, 2H), 3.69-3.59 (m, 1H), 3.45 (s, 3H), 3.26-3.17 (m, 1H), 2.78- 2.69 (m, 1H), 2.19-2.04 (m, 1H), 1.91-1.67 (m, 8H), 1.44 (d, J = 6.7 Hz, 3H), 1.25-0.99 (m, 2H) |
| D25 | 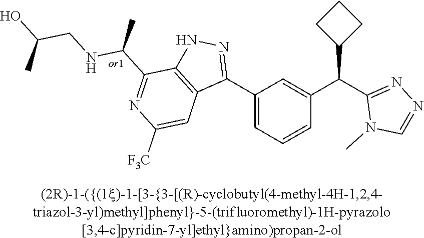 [SMILES: C[C@H](NC[C@@H](C)O)c1nc(C(F)(F)F)cc2c(-c3cccc([C@H](c4nncn4C)C4CCC4)c3)n[nH]c12] | 514.3 | ¹H NMR (400 MHz, DMSO- d₆) δ 8.33 (s, 1H), 8.21 (s, 1H), 7.87 (d, J = 7.7 Hz, 1H), 7.84 (s, 1H), 7.51 (t, J = 7.7 Hz, 1H), 7.33 (d, J = 7.7 Hz, 1H), 4.37-4.25 (m, 2H), 3.43 (s, 3H), 3.27-3.13 (m, 1H), 2.46 (br s, 1H), 2.26 (dd, J = 4.6, 11.4 Hz, 1H), 2.16-2.04 (m, 1H), 1.89-1.62 (m, 6H), 1.45 (d, J = 6.6 Hz, 3H), 0.99 (d, J = 6.2 Hz, 3H) |
| D26 | 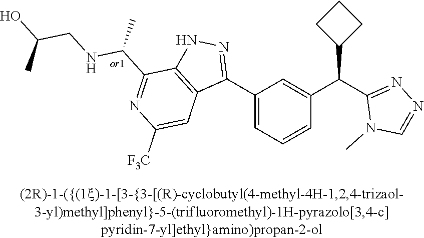 [SMILES: C[C@@H](O)CN[C@H](C)c1nc(C(F)(F)F)cc2c(-c3cccc([C@H](c4nncn4C)C4CCC4)c3)n[nH]c12] | 514.3 | ¹H NMR (400 MHz, DMSO- d₆) 8.33 (s, 1H), 8.21 (s, 1H), 7.87 (d, J = 7.5 Hz, 1H), 7.84 (s, 1H), 7.51 (t, J = 7.6 Hz, 1H), 7.34 (d, J = 7.9 Hz, 1H), 4.36-4.26 (m, 2H), 3.43 (s, 3H), 3.26-3.14 (m, 1H), 2.55 (br d, J = 2.0 Hz, 1H), 2.20- 2.05 (m, 2H), 1.91-1.66 (m, 6H), 1.45 (d, J = 6.8 Hz, 3H), 0.97 (d, J = 6.2 Hz, 3H) |
| D27 | 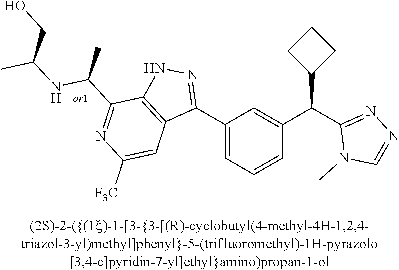 [SMILES: C[C@H](N[C@@H](C)CO)c1nc(C(F)(F)F)cc2c(-c3cccc([C@H](c4nncn4C)C4CCC4)c3)n[nH]c12] | 514.3 | ¹H NMR (400 MHz, DMSO- d₆) 8.32 (d, J = 4.8 Hz, 1H), 8.17 (br d, J = 4.6 Hz, 1H), 7.90-7.81 (m, 1H), 7.77 (br d, J = 7.0 Hz, 1H), 7.57-7.46 (m, 1H), 7.33 (br d, J = 1.5 Hz, 1H), 4.47 (br d, J = 6.8 Hz, 1H), 4.36-4.24 (m, 1H), 3.41 (br d, J = 4.4 Hz, 3H), 3.32 (br d, J = 4.4 Hz, 2H), 3.18 (br d, J = 7.3 Hz, 1H), 2.14- 2.03 (m, 1H), 1.88-1.58 (m, 6H), 1.45-1.34 (m, 3H), 0.79 (br d, J = 3.7 Hz, 3H) |
| D28 | 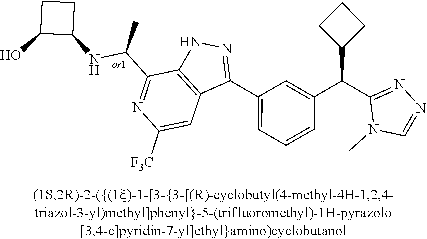 [SMILES: C[C@H](N[C@@H]1CC[C@@H]1O)c1nc(C(F)(F)F)cc2c(-c3cccc([C@H](c4nncn4C)C4CCC4)c3)n[nH]c12] | 526.2 | ¹H NMR (400 MHz, CD₃OD) δ 8.36 (s, 1H), 8.16 (s, 1H), 7.87 (td, J = 1.3, 7.8 Hz, 1H), 7.83 (t, J = 1.5 Hz, 1H), 7.54 (t, J = 7.7 Hz, 1H), 7.39-7.32 (m, 1H), 4.55 (q, J = 6.7 Hz, 1H), 4.39-4.29 (m, 2H), 3.53 (s, 3H), 3.33 (br s, 1H), 3.30- 3.26 (m, 1H), 2.36-2.22 (m, 1H), 2.07-1.97 (m, 2H), 1.95- 1.85 (m, 5H), 1.85-1.69 (m, 2H), 1.58 (d, J = 6.8 Hz, 3H) [α]_{D}²² −31.0° (c 0.1, MeOH) |
| D29 | 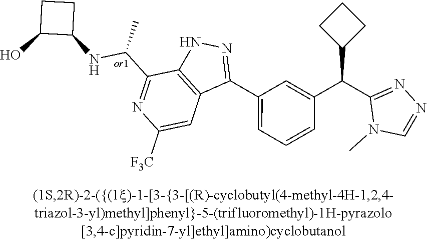 [SMILES: C[C@@H](N[C@@H]1CC[C@@H]1O)c1nc(C(F)(F)F)cc2c(-c3cccc([C@H](c4nncn4C)C4CCC4)c3)n[nH]c12] | 526.2 | ¹H NMR (400 MHz, CD₃OD) δ 3.24 (br s, 1H), 3.19-3.10 (m, 1H), 2.28-2.11 (m, 1H), 2.05-1.81 (m, 7H), 1.76-1.65 (m, 2H), 1.47 (d, J = 6.8 Hz, 3H) [α]_{D}²² −60.4° (c 0.1, MeOH) |
| D30 | 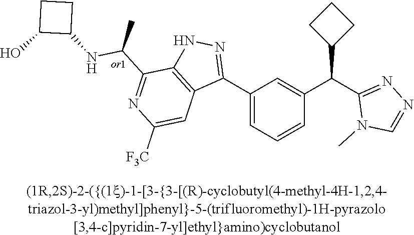 | 526.4 | ¹H NMR (400 MHz, CD₃OD) δ 8.37 (s, 1H), 8.13 (s, 1H), 7.87 (d, J = 7.9 Hz, 1H), 7.83 (s, 1H), 7.53 (t, J = 7.7 Hz, 1H), 7.34 (d, J = 7.7 Hz, 1H), 4.43 (q, J = 6.8 Hz, 1H), 4.39- 4.28 (m, 2H), 3.53 (s, 3H), 3.35 (br s, 1H), 3.25-3.15 (m, 1H), 2.38-2.23 (m, 1H), 2.02- 1.88 (m, 5H), 1.87-1.66 (m, 4H), 1.54 (d, J = 6.8 Hz, 3H) |
| D31 | 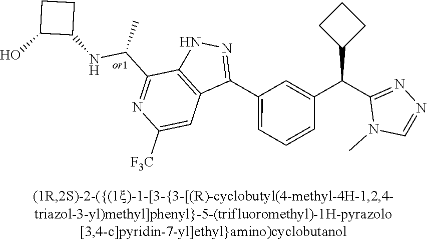 | 526.4 | ¹H NMR (400 MHz, CD₃OD) δ 8.37 (s, 1H), 8.14 (s, 1H), 7.87 (d, J = 7.7 Hz, 1H), 7.83 (s, 1H), 7.53 (t, J = 7.7 Hz, 1H), 7.34 (d, J = 7.9 Hz, 1H), 4.49 (q, J = 6.6 Hz, 1H), 4.33 (d, J = 10.8 Hz, 1H), 4.17- 4.09 (m, 1H), 3.53 (s, 3H), 3.38-3.32 (m, 1H), 3.29-3.21 (m, 1H), 2.35-2.22 (m, 1H), 2.11-1.88 (m, 7H), 1.86-1.76 (m, 2H), 1.56 (d, J = 6.6 Hz, 3H) |
| D34 | 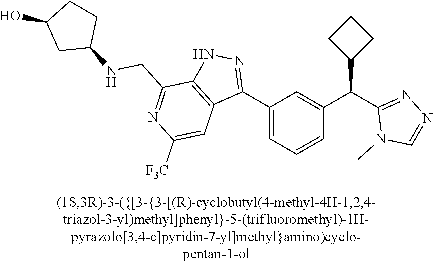 [SMILES: Cn1cnnc1[C@@H](c1cccc(-c2n[nH]c3c(CN[C@@H]4CC[C@H](O)C4)nc(C(F)(F)F)cc23)c1)C1CCC1] | 526.3 | ¹H NMR (400 MHz, CD₃OD) δ 8.37 (s, 1H), 8.16 (s, 1H), 7.89-7.81 (m, 2H), 7.53 (t, J = 7.7 Hz, 1H), 7.34 (d, J = 7.8 Hz, 1H), 4.37-4.22 (m, 4H), 3.53 (s, 3H), 2.34-2.23 (m, 1H), 2.22-2.11 (m, 1H), 2.06- 1.59 (m, 12H). |
| D35 | 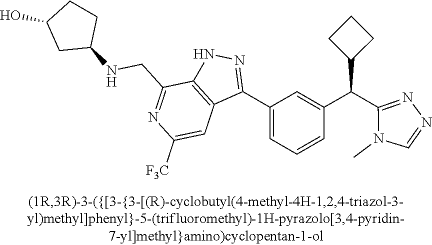 [SMILES: Cn1cnnc1[C@@H](c1cccc(-c2n[nH]c3c(CN[C@@H]4CC[C@@H](O)C4)nc(C(F)(F)F)cc23)c1)C1CCC1] | 526.3 | ¹H NMR (400 MHz, CD₃OD) δ 8.38 (s, 1H), 8.17 (s, 1H), 7.91-7.80 (m, 2H), 7.53 (t, J = 7.7 Hz, 1H), 7.34 (br d, J = 7.5 Hz, 1H), 4.41-4.27 (m, 4H), 3.53 (s, 3H), 3.47 (quin, J = 7.1 Hz, 1H), 2.35-2.25 (m, 1H), 2.20-1.42 (m, 12H). |
| D36 | 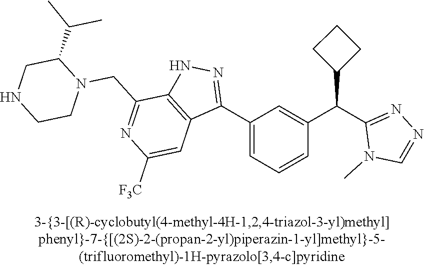 [SMILES: CC(C)[C@H]1CNCCN1Cc1nc(C(F)(F)F)cc2c(-c3cccc([C@H](c4nncn4C)C4CCC4)c3)n[nH]c12] | 553.3 | ¹H NMR (400 MHz, CDCl₃) δ 8.53 (br s, 1H), 8.17 (s, 1H), 8.14 (s, 1H), 7.82-7.77 (m, 2H), 7.50 (t, J = 7.8 Hz, 1H), 7.33 (br d, J = 7.9 Hz, 1H), 4.77 (br d, J = 15.8 Hz, 1H), 4.05-3.99 (m, 1H), 3.93 (br d, J = 15.2 Hz, 1H), 3.51 (s, 3H), 3.42-3.34 (m, 3H), 3.30-3.11 (m, 3H), 2.92 (br d, J = 12.5 Hz, 1H), 2.81-2.72 (m, 2H), 2.54-2.44 (m, 1H), 2.31 (br dd, J = 5.7, 8.8 Hz, 1H), 1.98- 1.80 (m, 4H), 1.79-1.69 (m, 1H), 1.05 (br t, J = 6.1 Hz, 6H). |
| D37 | 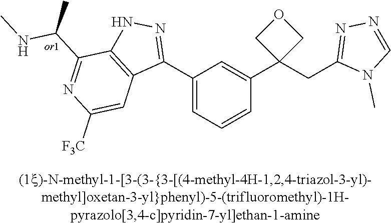 [SMILES: CN[C@@H](C)c1nc(C(F)(F)F)cc2c(-c3cccc(C4(Cc5nncn5C)COC4)c3)n[nH]c12] | 472.4 | ¹H NMR (400 MHz, CD₃OD) δ 8.26 (s, 1H), 8.17 (s, 1H), 7.93 (br d, J = 7.7 Hz, 1H), 7.59-7.48 (m, 2H), 7.05 (br d, J = 7.7 Hz, 1H), 5.19-5.10 (s, 4H), 4.65 (br d, J = 6.6 Hz, 1H), 3.71 (s, 2H), 2.93 (s, 3H), 2.56 (s, 3H), 1.65 (d, J = 6.7 Hz, 3H). |
| D38 | 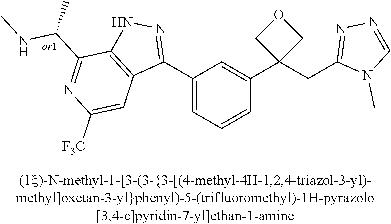 [SMILES: CN[C@H](C)c1nc(C(F)(F)F)cc2c(-c3cccc(C4(Cc5nncn5C)COC4)c3)n[nH]c12] | 472.4 | ¹H NMR (400 MHz, CD₃OD) δ 8.27 (s, 1H), 8.18 (s, 1H), 7.93 (d, J = 7.8 Hz, 1H), 7.60- 7.48 (m, 2H), 7.06 (d, J = 7.8 Hz, 1H), 5.18-5.10 (m, 4H), 4.73 (q, J = 6.7 Hz, 1H), 3.71 (s, 2H), 2.94 (s, 3H), 2.61 (s, 3H), 1.68 (d, J = 6.8 Hz, 3H). |
| D39 | 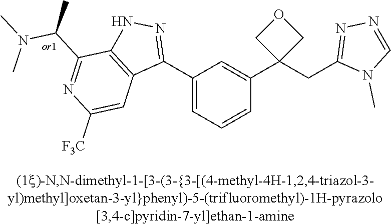 [SMILES: C[C@@H](c1nc(C(F)(F)F)cc2c(-c3cccc(C4(Cc5nncn5C)COC4)c3)n[nH]c12)N(C)C] | 486.3 | ¹H NMR (400 MHz, DMSO- d₆) δ 8.23 (s, 1H), 8.20 (s, 1H), 7.90 (d, J = 7.9 Hz, 1H), 7.59 (s, 1H), 7.49 (t, J = 7.7 Hz, 1H), 7.10 (d, J = 7.8 Hz, 1H), 5.02-4.92 (m, 4H), 4.18 (br s, 1H), 3.59 (s, 2H), 2.98 (s, 3H), 2.26 (br s, 6H), 1.46 (br d, J = 6.6 Hz, 3H). |
| D40 | 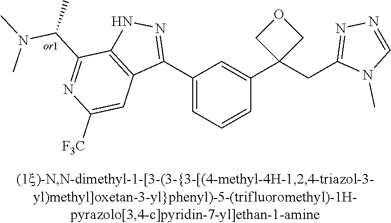 [SMILES: C[C@H](c1nc(C(F)(F)F)cc2c(-c3cccc(C4(Cc5nncn5C)COC4)c3)n[nH]c12)N(C)C] | 486.4 | ¹H NMR (400 MHz, DMSO- d₆) δ 8.22 (s, 1H), 8.20 (s, 1H), 7.90 (d, J = 7.9 Hz, 1H), 7.59 (s, 1H), 7.49 (t, J = 7.8 Hz, 1H), 7.10 (d, J = 7.8 Hz, 1H), 5.02-4.93 (m, 4H), 4.14 (q, J = 6.7 Hz, 1H), 3.59 (s, 2H), 2.97 (s, 3H), 2.23 (s, 6H), 1.45 (d, J = 6.8 Hz, 3H). |
| D41 | 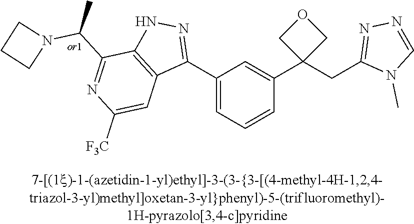 [SMILES: C[C@@H](c1nc(C(F)(F)F)cc2c(-c3cccc(C4(Cc5nncn5C)COC4)c3)n[nH]c12)N1CCC1] | 498.3 | ¹H NMR (400 MHz, CD₃OD) δ 8.16 (s, 1H), 8.15 (s, 1H), 7.90 (d, J = 7.9 Hz, 1H), 7.52 (t, J = 7.8 Hz, 1H), 7.47 (s, 1H), 6.99 (d, J = 8.4 Hz, 1H), 5.20-5.06 (m, 4H), 4.05 (q, J = 6.7 Hz, 1H), 3.69 (s, 2H), 3.43-3.36 (m, 2H), 3.27-3.21 (m, 2H), 2.88 (s, 3H), 2.23- 2.09 (m, 2H), 1.39 (d, J = 6.7 Hz, 3H). |
| D42 | 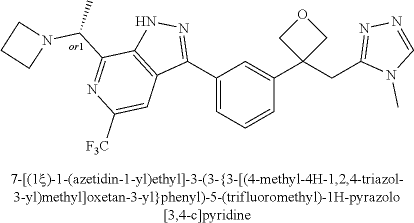 [SMILES: C[C@H](c1nc(C(F)(F)F)cc2c(-c3cccc(C4(Cc5nncn5C)COC4)c3)n[nH]c12)N1CCC1] | 498.3 | ¹H NMR (400 MHz, CD₃OD) δ 8.16 (s, 1H), 8.15 (s, 1H), 7.90 (d, J = 7.8 Hz, 1H), 7.52 (t, J = 7.8 Hz, 1H), 7.47 (s, 1H), 6.99 (d, J = 8.3 Hz, 1H), 5.15-5.09 (m, 4H), 4.06 (q, J = 6.6 Hz, 1H), 3.69 (s, 2H), 3.43-3.37 (m, 2H), 3.28-3.22 (m, 2H), 2.88 (s, 3H), 2.20- 2.09 (m, 2H), 1.40 (d, J = 6.7 Hz, 3H). |
| D43 | 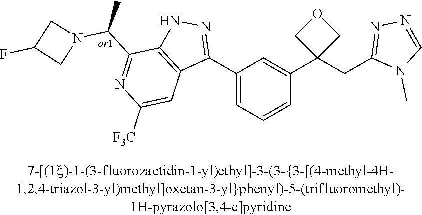 [SMILES: C[C@@H](c1nc(C(F)(F)F)cc2c(-c3cccc(C4(Cc5nncn5C)COC4)c3)n[nH]c12)N1CC(F)C1] | 516.3 | ¹H NMR (400 MHz, CD₃OD) δ 8.19 (s, 1H), 8.18 (s, 1H), 7.92 (d, J = 7.7 Hz, 1H), 7.54 (t, J = 7.7 Hz, 1H), 7.49 (s, 1H), 7.03 (d, J = 7.7 Hz, 1H), 5.35-5.26 (m, 1H), 5.17-5.11 (m, 4H), 4.14 (br s, 1H), 3.91 (br d, J = 7.2 Hz, 1H), 3.71 (s, 2H), 3.58 (br s, 1H), 3.50- 3.37 (m, 2H), 2.91 (s, 3H), 1.47 (d, J = 6.7 Hz, 3H). |
| D44 | 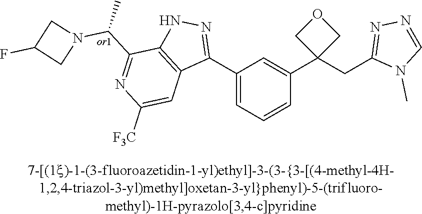 [SMILES: C[C@H](c1nc(C(F)(F)F)cc2c(-c3cccc(C4(Cc5nncn5C)COC4)c3)n[nH]c12)N1CC(F)C1] | 516.3 | ¹H NMR (400 MHz, CD₃OD) δ 8.16 (s, 1H), 8.15 (s, 1H), 7.90 (d, J = 7.8 Hz, 1H), 7.52 (t, J = 7.7 Hz, 1H), 7.46 (s, 1H), 7.00 (d, J = 7.8 Hz, 1H), 5.27 (quin, J = 5.2 Hz, 1H), 5.14-5.09 (m, 4H), 4.06 (q, J = 6.6 Hz, 1H), 3.85 (td, J = 7.1, 14.6 Hz, 1H), 3.69 (s, 2H), 3.55-3.44 (m, 1H), 3.35- 3.34 (m, 1H), 3.28-3.20 (m, 1H), 2.88 (s, 3H), 1.43 (d, J = 6.6 Hz, 3H). |
| D45 | 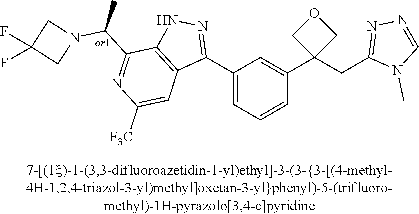 [SMILES: C[C@@H](c1nc(C(F)(F)F)cc2c(-c3cccc(C4(Cc5nncn5C)COC4)c3)n[nH]c12)N1CC(F)(F)C1] | 534.3 | ¹H NMR (400 MHz, CD₃OD) δ 8.19 (s, 1H), 8.16 (s, 1H), 7.91 (d, J = 7.7 Hz, 1H), 7.52 (t, J = 7.8 Hz, 1H), 7.47 (s, 1H), 7.00 (br d, J = 8.2 Hz, 1H), 5.19-5.08 (m, 4H), 4.21- 4.09 (m, 1H), 3.83-3.58 (m, 6H), 2.88 (s, 3H), 1.49 (d, J = 6.6 Hz, 3H). |
| D46 | 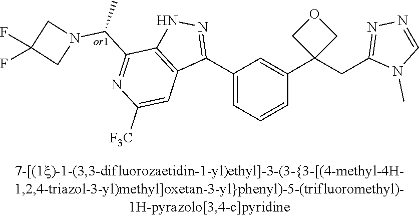 [SMILES: C[C@H](c1nc(C(F)(F)F)cc2c(-c3cccc(C4(Cc5nncn5C)COC4)c3)n[nH]c12)N1CC(F)(F)C1] | 534.3 | ¹H NMR (400 MHz, CD₃OD) δ 8.19 (s, 1H), 8.16 (s, 1H), 7.91 (d, J = 7.7 Hz, 1H), 7.52 (t, J = 7.8 Hz, 1H), 7.47 (s, 1H), 7.00 (d, J = 7.8 Hz, 1H), 5.18-5.08 (m, 4H), 4.15 (br d, J = 6.5 Hz, 1H), 3.83-3.57 (m, 6H), 2.88 (s, 3H), 1.49 (d, J = 6.6 Hz, 3H). |
| D47 | 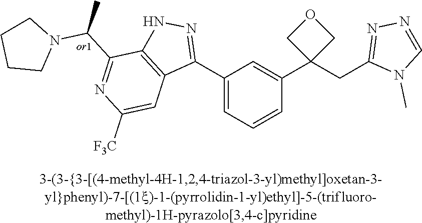 [SMILES: C[C@@H](c1nc(C(F)(F)F)cc2c(-c3cccc(C4(Cc5nncn5C)COC4)c3)n[nH]c12)N1CCCC1] | 512.4 | ¹H NMR (400 MHz, CD₃OD) δ 8.19 (s, 1H), 8.16 (s, 1H), 7.93 (d, J = 7.8 Hz, 1H), 7.54 (t, J = 7.7 Hz, 1H), 7.49 (s, 1H), 7.02 (d, J = 8.3 Hz, 1H), 5.21-5.11 (m, 4H), 4.05 (br d, J = 6.7 Hz, 1H), 3.71 (s, 2H), 2.90 (s, 3H), 2.83 (br d, J = 8.2 Hz, 2H), 2.53 (br d, J = 7.7 Hz, 2H), 1.95-1.80 (m, 4H), 1.61 (d, J = 6.8 Hz, 3H). |
| D48 | 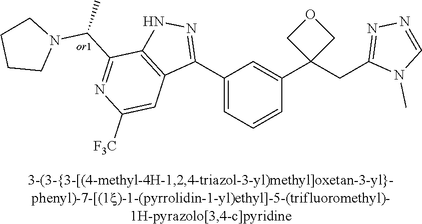 [SMILES: C[C@H](c1nc(C(F)(F)F)cc2c(-c3cccc(C4(Cc5nncn5C)COC4)c3)n[nH]c12)N1CCCC1] | 512.4 | ¹H NMR (400 MHz, CD₃OD) δ 8.19 (s, 1H), 8.18 (s, 1H), 7.93 (d, J = 7.8 Hz, 1H), 7.54 (t, J = 7.8 Hz, 1H), 7.49 (s, 1H), 7.02 (d, J = 8.3 Hz, 1H), 5.21-5.07 (m, 4H), 4.11-3.98 (m, 1H), 3.71 (s, 2H), 2.90 (s, 3H), 2.86-2.78 (m, 2H), 2.54- 2.52 (m, 2H), 1.95-1.80 (m, 4H), 1.61 (d, J = 6.7 Hz, 3H). |
| D49 | 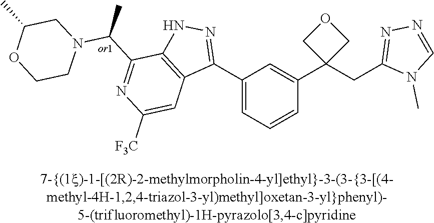 [SMILES: C[C@@H]1CN([C@@H](C)c2nc(C(F)(F)F)cc3c(-c4cccc(C5(Cc6nncn6C)COC5)c4)n[nH]c23)CCO1] | 542.4 | ¹H NMR (400 MHz, CD₃OD) δ 8.19 (s, 1H), 8.18 (s, 1H), 7.93 (d, J = 7.8 Hz, 1H), 7.55 (t, J = 7.8 Hz, 1H), 7.50 (s, 1H), 7.02 (d, J = 7.8 Hz, 1H), 5.20-5.10 (m, 4H), 4.08 (q, J = 6.4 Hz, 1H), 3.84-3.65 (m, 5H), 3.01 (br d, J = 11.1 Hz, 1H), 2.91 (s, 3H), 2.56-2.49 (m, 1H), 2.45-2.35 (m, 1H), 1.97 (t, J = 10.5 Hz, 1H), 1.56 (d, J = 6.7 Hz, 3H), 1.16 (d, J = 6.4 Hz, 3H). |
| D50 | 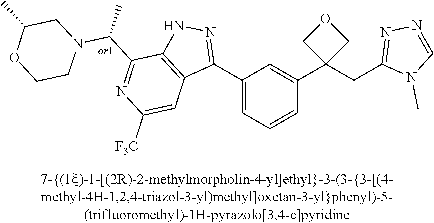 [SMILES: C[C@@H]1CN([C@H](C)c2nc(C(F)(F)F)cc3c(-c4cccc(C5(Cc6nncn6C)COC5)c4)n[nH]c23)CCO1] | 542.4 | ¹H NMR (400 MHz, CD₃OD) δ 8.19 (s, 1H), 8.18 (s, 1H), 7.93 (d, J = 7.6 Hz, 1H), 7.55 (t, J = 7.8 Hz, 1H), 7.50 (s, 1H), 7.02 (br d, J = 7.9 Hz, 1H), 5.24-5.06 (m, 4H), 4.07 (q, J = 6.6 Hz, 1H), 3.97-3.77 (m, 2H), 3.76-3.59 (m, 3H), 2.97-2.94 (m, 1H), 2.91 (s, 3H), 2.57 (br d, J = 11.4 Hz, 1H), 2.30 (dt, J = 3.5, 11.3 Hz, 1H), 2.15-2.01 (m, 1H), 1.56 (d, J = 6.7 Hz, 3H), 1.04 (d, J = 6.4 Hz, 3H). |
| D51 | 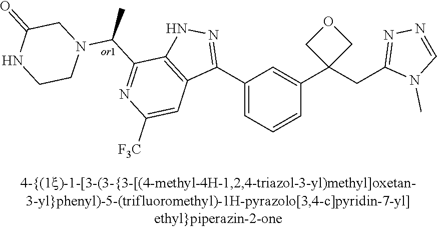 [SMILES: C[C@@H](c1nc(C(F)(F)F)cc2c(-c3cccc(C4(Cc5nncn5C)COC4)c3)n[nH]c12)N1CCNC(=O)C1] | 541.3 | ¹H NMR (400 MHz CD₃OD) δ 8.18 (s, 1H), 8.18 (s, 1H), 7.90 (d, J = 7.9 Hz, 1H), 7.52 (t, J = 7.8 Hz, 1H), 7.48 (s, 1H), 7.00 (d, J = 8.1 Hz, 1H), 5.18-5.09 (m, 4H), 4.24 (q, J = 6.7 Hz, 1H), 3.69 (s, 2H), 3.52 (d, J = 16.5 Hz, 1H), 3.29-3.16 (m, 3H), 2.89 (s, 3H), 2.79-2.62 (m, 2H), 1.59 (d, J = 6.8 Hz, 3H). |
| D52 | 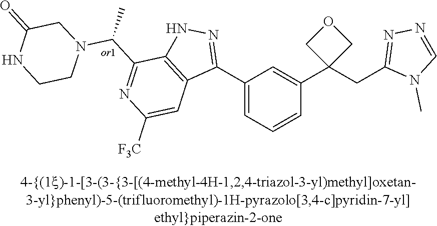 [SMILES: C[C@H](c1nc(C(F)(F)F)cc2c(-c3cccc(C4(Cc5nncn5C)COC4)c3)n[nH]c12)N1CCNC(=O)C1] | 541.3 | ¹H NMR (400 MHz, CD₃OD) δ 8.19 (s, 1H), 8.16 (s, 1H), 7.90 (d, J = 7.8 Hz, 1H), 7.52 (t, J = 7.8 Hz, 1H), 7.48 (s, 1H), 7.00 (d, J = 7.8 Hz, 1H), 5.18-5.08 (m, 4H), 4.25 (q, J = 6.7 Hz, 1H), 3.69 (s, 2H), 3.52 (d, J = 16.4 Hz, 1H), 3.29-3.16 (m, 3H), 2.89 (s, 3H), 2.80-2.59 (m, 2H), 1.59 (d, J = 6.8 Hz, 3H). |
| D53 | 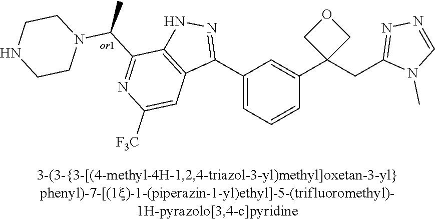 [SMILES: C[C@@H](c1nc(C(F)(F)F)cc2c(-c3cccc(C4(Cc5nncn5C)COC4)c3)n[nH]c12)N1CCNCC1] | 527.4 | ¹H NMR (400 MHz, CD₃OD) δ 8.19 (s, 2H), 7.93 (d, J = 8.0 Hz, 1H), 7.58-7.47 (m, 2H), 7.03 (d, J = 7.8 Hz, 1H), 5.17- 5.11 (m, 4H), 4.14 (q, J = 6.9 Hz, 1H), 3.71 (s, 2H), 2.96 (t, J = 4.8 Hz, 4H), 2.91 (s, 3H), 2.80-2.70 (m, 2H), 2.56-2.44 (m, 2H), 1.57 (d, J = 6.8 Hz, 3H). |
| D54 | 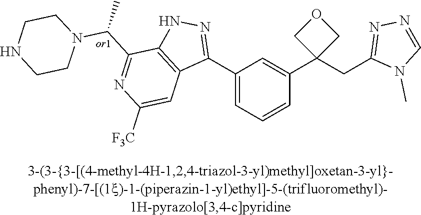 [SMILES: C[C@H](c1nc(C(F)(F)F)cc2c(-c3cccc(C4(Cc5nncn5C)COC4)c3)n[nH]c12)N1CCNCC1] | 527.3 | ¹H NMR (400 MHz, CD₃OD) δ 8.19 (s, 2H), 7.93 (d, J = 7.8 Hz, 1H), 7.58-7.46 (m, 2H), 7.02 (d, J = 7.8 Hz, 1H), 5.19- 5.10 (m, 4H), 4.14 (q, J = 6.8 Hz, 1H), 3.71 (s, 2H), 2.95 (t, J = 4.8 Hz, 4H), 2.91 (s, 3H), 2.80-2.70 (m, 2H), 2.55-2.42 (m, 2H), 1.57 (d, J = 6.8 Hz, 3H). |
| D55 | 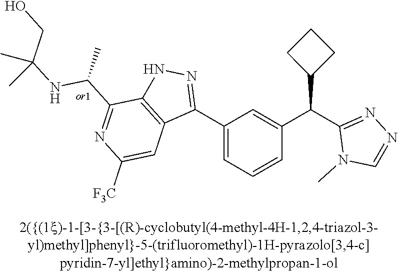 [SMILES: C[C@@H](NC(C)(C)CO)c1nc(C(F)(F)F)cc2c(-c3cccc([C@H](c4nncn4C)C4CCC4)c3)n[nH]c12] | 528.3 | ¹H NMR (400 MHz, CD₃OD) δ 8.38 (s, 1H), 8.11 (s, 1H), 7.88 (d, J = 7.9 Hz, 1H), 7.84 (s, 1H), 7.54 (t, J = 7.6 Hz, 1H), 7.35 (d, J = 6.8 Hz, 1H), 4.69-4.54 (m, 3H), 4.34 (d, J = 10.8 Hz, 1H), 3.54 (s, 3H), 3.45 (d, J = 10.8 Hz, 1H), 3.35-3.33 (m, 2H), 2.34- 2.25 (m, 1H), 2.03-1.86 (m, 5H), 1.52 (d, J = 6.8 Hz, 3H), 1.00 (s, 3H), 0.87 (s, 3H). |
| D56 | 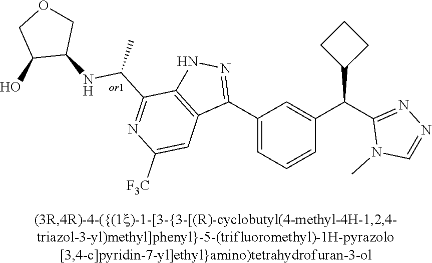 [SMILES: C[C@@H](N[C@@H]1COC[C@@H]1O)c1nc(C(F)(F)F)cc2c(-c3cccc([C@H](c4nncn4C)C4CCC4)c3)n[nH]c12] | 542.4 | ¹H NMR (400 MHz, CD₃OD) δ 8.37 (s, 1H), 8.17 (s, 1H), 7.88 (d, J = 7.8 Hz, 1H), 7.83 (s, 1H), 7.54 (t, J = 7.7 Hz, 1H), 7.35 (d, J = 7.6 Hz, 1H), 4.53 (q, J = 6.5 Hz, 1H), 4.33 (d, J = 10.9 Hz, 1H), 4.04-3.96 (m, 2H), 3.86 (dd, J = 4.4, 9.8 Hz, 1H), 3.73 (dd, J = 1.9, 9.8 Hz, 1H), 3.65 (t, J = 8.3 Hz, 1H), 3.53 (s, 3H), 3.29-3.22 (m, 1H), 2.34-2.23 (m, 1H), 2.04-1.85 (m, 5H), 1.85-1.73 (m, 1H), 1.59 (d, J = 6.8 Hz, 3H). |
| D57 | 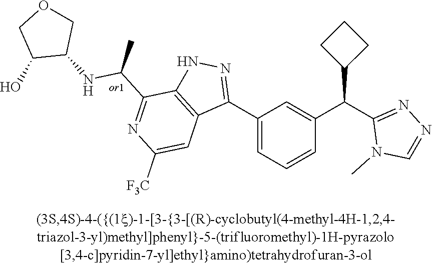 [SMILES: C[C@H](N[C@H]1COC[C@H]1O)c1nc(C(F)(F)F)cc2c(-c3cccc([C@H](c4nncn4C)C4CCC4)c3)n[nH]c12] | 542.4 | ¹H NMR (400 MHz, CD₃OD) δ 8.37 (s, 1H), 8.16 (s, 1H), 7.87 (br d, J = 6.9 Hz, 1H), 7.82 (br s, 1H), 7.54 (br t, J = 7.6 Hz, 1H), 7.35 (br d, J = 7.6 Hz, 1H), 4.52-4.47 (m, 1H), 4.33 (br d, J = 11.1 Hz, 1H), 4.06-3.94 (m, 2H), 3.91-3.83 (m, 1H), 3.78-3.69 (m, 1H), 3.68-3.60 (m, 1H), 3.53 (s, 3H), 3.26-3.18 (m, 1H), 2.37- 2.23 (m, 1H), 2.07-1.86 (m, 4H), 1.85-1.73 (m, 1H), 1.58 (br d, J = 6.5 Hz, 3H). |
| D58 | 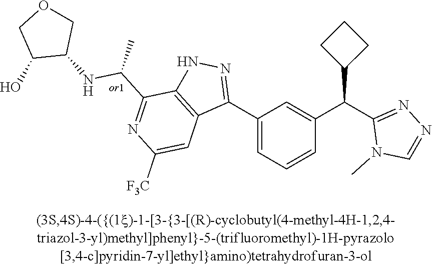 [SMILES: C[C@@H](N[C@H]1COC[C@H]1O)c1nc(C(F)(F)F)cc2c(-c3cccc([C@H](c4nncn4C)C4CCC4)c3)n[nH]c12] | 542.4 | ¹H NMR (400 MHz, CD₃OD) δ 8.38 (s, 1H), 8.19 (s, 1H), 7.88 (br d, J = 8.1 Hz, 1H), 7.84 (s, 1H), 7.55 (t, J = 7.8 Hz, 1H), 7.37 (br d, J = 7.7 Hz, 1H), 4.37-4.29 (m, 2H), 3.89-3.83 (m, 1H), 3.81-3.70 (m, 2H), 3.62-3.56 (m, 1H), 3.54 (s, 3H), 3.37-3.34 (m, 1H), 3.29-3.19 (m, 2H), 2.35- 2.23 (m, 1H), 2.01-1.88 (m, 4H), 1.85-1.75 (m, 1H), 1.63 (br d, J = 6.6 Hz, 3H). |
| D59 | 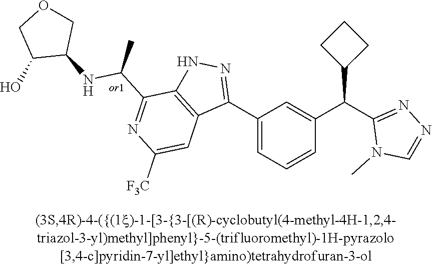 [SMILES: C[C@H](N[C@@H]1COC[C@H]1O)c1nc(C(F)(F)F)cc2c(-c3cccc([C@H](c4nncn4C)C4CCC4)c3)n[nH]c12] | 542.4 | ¹H NMR (400 MHz, DMSO- d₆) δ 8.34 (s, 1H), 8.25 (s, 1H), 7.93-7.87 (m, 2H), 7.51 (t, J = 8.0 Hz, 1H), 7.34 (d, J = 8.4 Hz, 1H), 4.89 (br d, J = 3.5 Hz, 1H), 4.55-4.46 (m, 1H), 4.37 (d, J = 10.8 Hz, 1H), 4.16-4.10 (m, 1H), 3.92- 3.86 (m, 1H), 3.73-3.65 (m, 1H), 3.49-3.47 (m, 1H), 3.45 (s, 3H), 3.44-3.42 (m, 1H), 3.24-3.17 (m, 1H), 2.89-2.84 (m, 1H), 2.15-2.06 (m, 1H), 1.92-1.66 (m, 5H), 1.45 (br d, J = 6.6 Hz, 3H). |
| D60 | 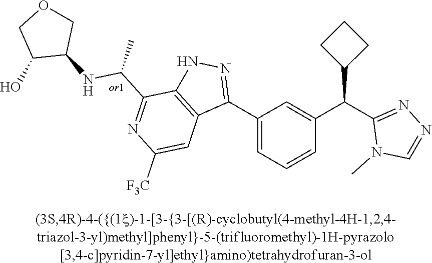 [SMILES: C[C@@H](N[C@@H]1COC[C@H]1O)c1nc(C(F)(F)F)cc2c(-c3cccc([C@H](c4nncn4C)C4CCC4)c3)n[nH]c12] | 542.4 | ¹H NMR (400 MHz, DMSO- d₆) δ 8.34 (s, 1H), 8.25 (s, 1H), 7.90 (br d, J = 1.8 Hz, 2H), 7.51 (t, J = 7.9 Hz, 1H), 7.34 (d, J = 7.6 Hz, 1H), 4.89-4.76 (m, 1H), 4.47-4.33 (m, 2H), 3.92-3.85 (m, 1H), 3.82-3.72 (m, 2H), 3.66-3.60 (m, 1H), 3.45 (s, 3H), 3.41-3.37 (m, 1H), 3.25-3.15 (m, 1H), 2.91- 2.85 (m, 1H), 2.17-2.08 (m, 1H), 1.92-1.68 (m, 5H), 1.46 (d, J = 6.8 Hz, 3H). |
| D61 | 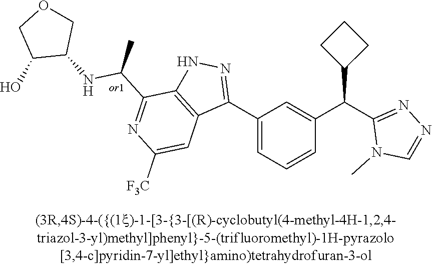 [SMILES: C[C@H](N[C@H]1COC[C@H]1O)c1nc(C(F)(F)F)cc2c(-c3cccc([C@H](c4nncn4C)C4CCC4)c3)n[nH]c12] | 542.4 | ¹H NMR (400 MHz, DMSO- d₆) δ 8.35 (s, 1H), 8.26 (s, 1H), 7.93-7.87 (m, 2H), 7.52 (t, J = 8.0 Hz, 1H), 7.35 (d, J = 7.8 Hz, 1H), 4.85 (br d, J = 3.5 Hz, 1H), 4.45-4.40 (m, 1H), 4.38 (d, J = 10.5 Hz, 1H), 3.92-3.86 (m, 1H), 3.77 (td, J = 4.7, 9.0 Hz, 2H), 3.66-3.60 (m, 1H), 3.45 (s, 3H), 3.23- 3.19 (m, 1H), 2.91-2.86 (m, 1H), 2.16-2.06 (m, 1H), 1.91- 1.68 (m, 5H), 1.46 (d, J = 6.8 Hz, 3H). |
| D62 | 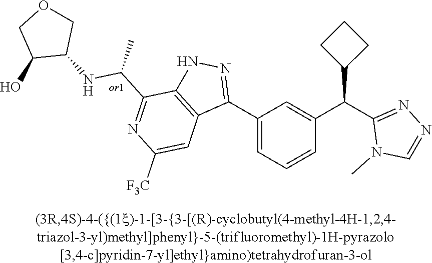 [SMILES: C[C@@H](N[C@H]1COC[C@@H]1O)c1nc(C(F)(F)F)cc2c(-c3cccc([C@H](c4nncn4C)C4CCC4)c3)n[nH]c12] | 542.3 | ¹H NMR (400 MHz, DMSO- d₆) δ 8.28 (s, 1H), 8.25 (s, 1H), 7.92-7.87 (m, 2H), 7.51 (t, J = 8.0 Hz, 1H), 7.34 (d, J = 7.8 Hz, 1H), 4.89 (br d, J = 2.6 Hz, 1H), 4.50 (q, J = 6.5 Hz, 1H), 4.37 (d, J = 10.5 Hz, 1H), 4.13 (br s, 1H), 3.88 (dd, J = 4.4, 9.0 Hz, 1H), 3.69 (dd, J = 5.4, 8.7 Hz, 1H), 3.47 (br d, J = 1.6 Hz, 1H), 3.44 (s, 3H), 3.42 (br d, J = 2.9 Hz, 1H), 3.21 (br d, J = 10.4 Hz, 1H), 2.87 (br s, 1H), 2.15-2.04 (m, 1H), 1.91-1.67 (m, 5H), 1.44 (d, J = 6.6 Hz, |
| | | | 3H). |
| D63 | 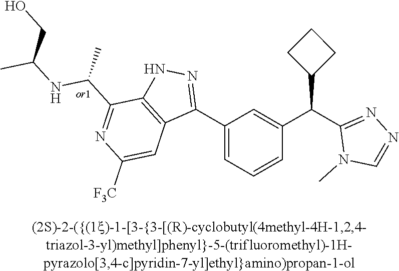 [SMILES: C[C@@H](CO)N[C@H](C)c1nc(C(F)(F)F)cc2c(-c3cccc([C@H](c4nncn4C)C4CCC4)c3)n[nH]c12] | 514.3 | ¹H NMR (400 MHz, DMSO- d₆ + D₂O) 0 8.32 (s, 1H), 8.18 (s, 1H), 7.85 (d, J = 7.7 Hz, 1H), 7.79 (s, 1H), 7.51 (t, J = 7.7 Hz, 1H), 7.33 (d, J = 7.7 Hz, 1H), 4.47 (q, J = 6.9 Hz, 1H), 4.31 (d, J = 10.6 Hz, 1H), 3.42 (s, 3H), 3.28-3.14 (m, 3H), 2.38-2.32 (m, 1H), 2.14- 2.04 (m, 1H), 1.87-1.64 (m, 5H), 1.43 (d, J = 6.8 Hz, 3H), 0.94 (d, J = 6.4 Hz, 3H). |
| D64 | 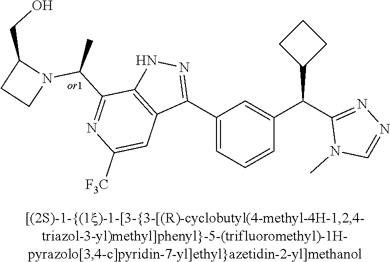 [SMILES: C[C@@H](c1nc(C(F)(F)F)cc2c(-c3cccc([C@H](c4nncn4C)C4CCC4)c3)n[nH]c12)N1CC[C@H]1CO] | 526.2 | ¹H NMR (600 MHz, DMSO- d₆) δ 8.33 (s, 1H), 8.24 (s, 1H), 7.91-7.89 (m, 2H), 7.50 (t, J = 7.9 Hz, 1H), 7.33 (d, J = 7.7 Hz, 1H), 4.37 (d, J = 10.4 Hz, 1H), 4.12-4.08 (m, 1H), 3.44 (s, 3H), 3.37-3.36 (m, 2H), 3.21 (br s, 1H), 3.12- 3.09 (m, 1H), 2.92 (dd, J = 6.0, 11.1 Hz, 1H), 2.66 (dd, J = 3.7, 11.0 Hz, 1H), 2.14- 2.07 (m, 1H), 1.98-1.94 (m, 2H), 1.89-1.77 (m, 4H), 1.77- 1.70 (m, 1H), 1.36 (d, J = 6.7 Hz, 3H). [α]_{D}²² −61.3° (c 0.1, MeOH) |
| D65 | 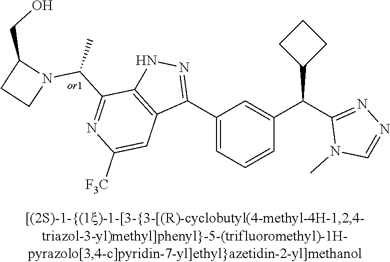 [SMILES: C[C@H](c1nc(C(F)(F)F)cc2c(-c3cccc([C@H](c4nncn4C)C4CCC4)c3)n[nH]c12)N1CC[C@H]1CO] | 526.2 | ¹H NMR (600 MHz, DMSO- d₆) δ 8.34 (s, 1H), 8.23 (s, 1H), 7.89 (br d, J = 1.9 Hz, 2H), 7.54-7.47 (m, 1H), 7.33 (d, J = 7.7 Hz, 1H), 4.36 (d, J = 10.5 Hz, 1H), 4.06-4.04 (m, 1H), 3.66 (dd, J = 3.9, 11.9 Hz, 1H), 3.59-3.53 (m, 1H), 3.47 (br dd, J = 2.8, 12.0 Hz, 1H), 3.44 (s, 3H), 3.21 (br s, 1H), 2.91-2.87 (m, 1H), 2.84-2.78 (m, 1H), 2.14-2.06 (m, 2H), 1.94-1.78 (m, 5H), 1.76-1.69 (m, 1H), 1.35 (d, J = 6.7 Hz, 3H). [α]_{D}²² −60.7° (c 0.1, MeOH) |
| D66 | 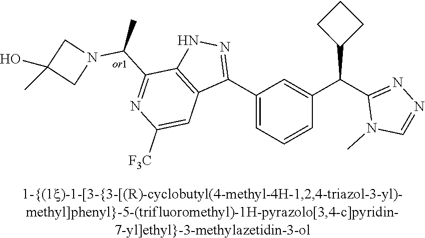 [SMILES: C[C@@H](c1nc(C(F)(F)F)cc2c(-c3cccc([C@H](c4nncn4C)C4CCC4)c3)n[nH]c12)N1CC(C)(O)C1] | 526.2 | ¹H NMR (600 MHz, DMSO- d₆) δ 8.34 (s, 1H), 8.24 (s, 1H), 7.89 (br d, J = 1.9 Hz, 2H), 7.53-7.48 (m, 1H), 7.34 (d, J = 7.7 Hz, 1H), 5.24 (br s, 1H), 4.36 (d, J = 10.5 Hz, 1H), 3.91 (br d, J = 6.3 Hz, 1H), 3.44 (s, 3H), 3.35-3.35 (m, 1H), 3.23-3.22 (m, 1H), 3.01 (br d, J = 6.3 Hz, 1H), 2.96-2.89 (m, 2H), 2.13-2.08 (m, 1H), 1.88-1.77 (m, 4H), 1.76-1.70 (m, 1H), 1.40 (s, 3H), 1.33 (br d, J = 6.4 Hz, 3H). [α]_{D}²² −113.7° (c 0.1, |
| | | | MeOH) |
| D67 | 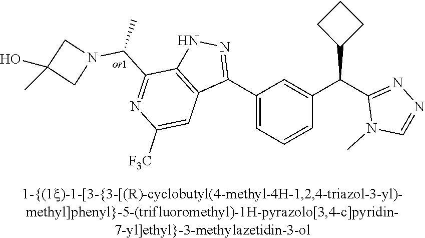 [SMILES: C[C@H](c1nc(C(F)(F)F)cc2c(-c3cccc([C@H](c4nncn4C)C4CCC4)c3)n[nH]c12)N1CC(C)(O)C1] | 526.2 | ¹H NMR (600 MHz, DMSO- d₆) δ 8.33 (s, 1H), 8.23 (s, 1H), 7.89 (td, J = 1.5, 3.1 Hz, 2H), 7.52-7.48 (m, 1H), 7.33 (d, J = 8.0 Hz, 1H), 5.25 (br s, 1H), 4.36 (d, J = 10.5 Hz, 1H), 3.93-3.89 (m, 1H), 3.44 (s, 3H), 3.35-3.34 (m, 1H), 3.23- 3.22 (m, 1H), 3.04-2.99 (m, 1H), 2.97-2.93 (m, 1H), 2.92- 2.88 (m, 1H), 2.13-2.08 (m, 1H), 1.89-1.76 (m, 4H), 1.76- 1.69 (m, 1H), 1.40 (s, 3H), 1.33 (d, J = 6.7 Hz, 3H). [α]_{D}²² −42.3° (c 0.1, MeOH) |
| D68 | 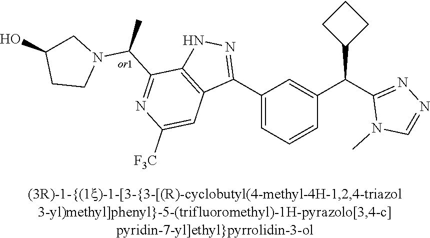 [SMILES: C[C@@H](c1nc(C(F)(F)F)cc2c(-c3cccc([C@H](c4nncn4C)C4CCC4)c3)n[nH]c12)N1CC[C@@H](O)C1] | 526.2 | ¹H NMR (600 MHz, DMSO- d₆) δ 8.33 (s, 1H), 8.25 (s, 1H), 7.92-7.87 (m, 2H), 7.51 (t, J = 7.9 Hz, 1H), 7.34 (d, J = 7.7 Hz, 1H), 4.87 (br d, J = 4.9 Hz, 1H), 4.36 (d, J = 10.5 Hz, 1H), 4.18 (br s, 1H), 3.95 (q, J = 6.7 Hz, 1H), 3.44 (s, 3H), 3.21 (br s, 1H), 2.67- 2.63 (m, 1H), 2.59-2.51 (m, 1H), 2.51-2.51 (m, 1H), 2.13 -2.08 (m, 1H), 2.08-2.02 (m, 1H), 1.85-1.78 (m, 2H), 1.89- 1.77 (m, 2H), 1.76-1.70 (m, 1H), 1.61-1.55 (m, 1H), 1.46 |
| | | | (d, J = 6.8 Hz, 3H). [α]_{D}²² |
| | | | −69.2° (c 0.1, MeOH) |
| D69 | 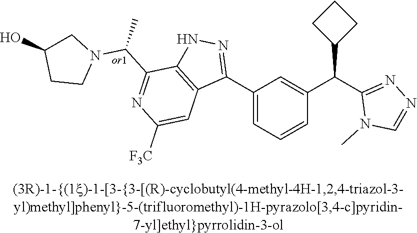 [SMILES: C[C@H](c1nc(C(F)(F)F)cc2c(-c3cccc([C@H](c4nncn4C)C4CCC4)c3)n[nH]c12)N1CC[C@@H](O)C1] | 526.2 | ¹H NMR (600 MHz, DMSO- d₆) δ 8.33 (s, 1H), 8.24 (s, 1H), 7.89 (td, J = 1.5, 3.1 Hz, 2H), 7.53-7.48 (m, 1H), 7.33 (d, J = 7.7 Hz, 1H), 4.36 (d, J = 10.4 Hz, 1H), 4.20 (br s, 1H), 4.01-3.97 (m, 1H), 3.44 (s, 3H), 3.21 (br s, 1H), 2.86- 2.82 (m, 1H), 2.78 (dd, J = 5.7, 9.4 Hz, 1H), 2.34-2.28 (m, 2H), 2.13-2.08 (m, 1H), 2.06-2.00 (m, 1H), 1.89-1.77 (m, 4H), 1.77-1.70 (m, 1H), 1.65-1.59 (m, 1H), 1.47 (d, |
| | | | J = 6.8 Hz, 3H). [α]_{D}²² −66.3° |
| | | | (c 0.1, MeOH) |
| D70 | 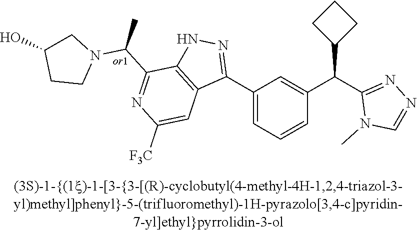 [SMILES: C[C@@H](c1nc(C(F)(F)F)cc2c(-c3cccc([C@H](c4nncn4C)C4CCC4)c3)n[nH]c12)N1CC[C@H](O)C1] | 526.2 | ¹H NMR (600 MHz, DMSO- d₆) δ 8.34 (s, 1H), 8.25 (s, 1H), 7.92-7.88 (m, 2H), 7.53- 7.49 (m, 1H), 7.34 (d, J = 7.7 Hz, 1H), 4.36 (d, J = 10.4 Hz, 1H), 4.18 (br s, 1H), 3.96 (q, J = 6.7 Hz, 1H), 3.44 (s, 3H), 3.21 (br s, 1H), 2.68-2.63 (m, 1H), 2.60-2.52 (m, 2H), 2.51- 2.51 (m, 1H), 2.14-2.08 (m, 1H), 2.06 (br dd, J = 5.8, 13.7 Hz, 1H), 1.89-1.78 (m, 4H), 1.77-1.71 (m, 1H), 1.59 (br dd, J = 5.3, 8.1 Hz, 1H), 1.49- |
| | | | 1.44 (m, 3H). [α]_{D}²² −75.7° |
| | | | (c 0.1, MeOH) |
| D71 | 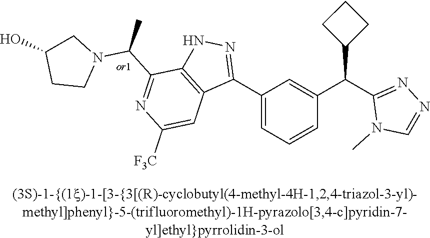 [SMILES: C[C@@H](c1nc(C(F)(F)F)cc2c(-c3cccc([C@H](c4nncn4C)C4CCC4)c3)n[nH]c12)N1CC[C@H](O)C1] | 526.2 | ¹H NMR (600 MHz, DMSO- d₆) δ 8.33 (s, 1H), 8.24 (s, 1H), 7.91-7.87 (m, 2H), 7.51 (t, J = 7.9 Hz, 1H), 7.34 (d, J = 7.7 Hz, 1H), 4.36 (d, J = 10.4 Hz, 1H), 4.20 (br s, 1H), 3.98 (br d, J = 6.8 Hz, 1H), 3.44 (s, 3H), 3.21 (br s, 1H), 2.87-2.83 (m, 1H), 2.78 (dd, J = 5.7, 9.4 Hz, 1H), 2.34- 2.28 (m, 2H), 2.13-2.08 (m, 1H), 2.06-2.00 (m, 1H), 1.87- 1.78 (m, 4H), 1.76-1.70 (m, 1H), 1.64-1.59 (m, 1H), 1.47 (d, J = 6.7 Hz, 3H). [α]_{D}²² |
| | | | −85.1° (c 0.1, MeOH) |
| D72 | 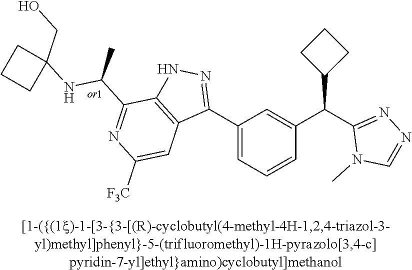 [SMILES: C[C@H](NC1(CO)CCC1)c1nc(C(F)(F)F)cc2c(-c3cccc([C@H](c4nncn4C)C4CCC4)c3)n[nH]c12] | 540.2 | ¹H NMR (600 MHz, DMSO- d₆) δ 8.33 (s, 1H), 8.19 (s, 1H), 7.89 (br d, J = 1.9 Hz, 2H), 7.50 (s, 1H), 7.33 (d, J = 7.7 Hz, 1H), 5.15-5.00 (m, 1H), 4.54 (br d, J = 6.8 Hz, 1H), 4.36 (d, J = 10.4 Hz, 1H), 3.49 (br s, 2H), 3.44 (s, 3H), 3.20 (br d, J = 2.5 Hz, 1H), 2.13-2.08 (m, 1H), 1.93- 1.77 (m, 5H), 1.75-1.68 (m, 3H), 1.67-1.61 (m, 1H), 1.58- 1.50 (m, 2H), 1.47 (d, J = 6.8 Hz, 3H). [α]_{D}²² −47.6° (c 0.1, MeOH) |
| D73 | 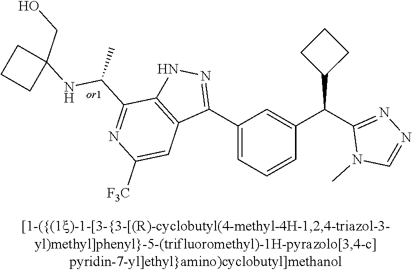 [SMILES: C[C@@H](NC1(CO)CCC1)c1nc(C(F)(F)F)cc2c(-c3cccc([C@H](c4nncn4C)C4CCC4)c3)n[nH]c12] | 540.2 | ¹H NMR (600 MHz, DMSO- d₆) δ 8.33 (s, 1H), 8.20 (s, 1H), 7.89 (s, 2H), 7.50 (t, J = 7.9 Hz, 1H), 7.33 (d, J = 7.7 Hz, 1H), 5.09 (br s, 1H), 4.54 (d, J = 6.8 Hz, 1H), 4.36 (d, J = 10.5 Hz, 1H), 3.49 (br s, 2H), 3.44 (s, 3H), 3.22 (br s, 1H), 2.11 (br dd, J = 3.5, 6.9 Hz, 1H), 1.93-1.76 (m, 5H), 1.75-1.68 (m, 3H), 1.67-1.61 (m, 1H), 1.58-1.50 (m, 2H), 1.50-1.45 (m, 3H). [α]_{D}²² −82.4° (c 0.1, MeOH) |
| D74 | 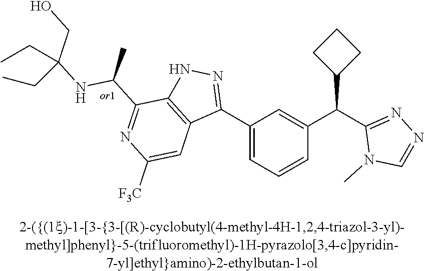 [SMILES: CCC(CC)(CO)N[C@@H](C)c1nc(C(F)(F)F)cc2c(-c3cccc([C@H](c4nncn4C)C4CCC4)c3)n[nH]c12] | 556.3 | ¹H NMR (600 MHz, DMSO- d₆) δ 8.33 (s, 1H), 8.18 (s, 1H), 7.89 (s, 2H), 7.50 (s, 1H), 7.36-7.29 (m, 1H), 4.45 (q, J = 6.7 Hz, 1H), 4.36 (d, J = 10.4 Hz, 1H), 3.45 (s, 3H), 3.23-3.20 (m, 2H), 3.12-3.07 (m, 1H), 2.16-2.06 (m, 1H), 1.88-1.77 (m, 4H), 1.76-1.69 (m, 1H), 1.44 (d, J = 6.9 Hz, 3H), 1.28-1.20 (m, 4H), 0.65- 0.57 (m, 6H). [α]_{D}²² −78.7° (c 0.1, MeOH) |
| D75 | 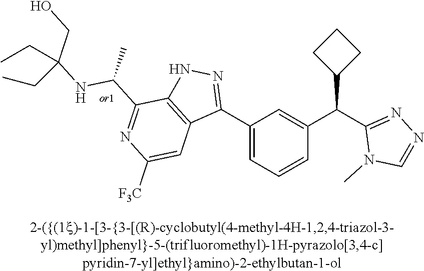 [SMILES: CCC(CC)(CO)N[C@H](C)c1nc(C(F)(F)F)cc2c(-c3cccc([C@H](c4nncn4C)C4CCC4)c3)n[nH]c12] | 556.3 | ¹H NMR (600 MHz, DMSO- d₆) δ 19.71-19.66 (m, 1H), 8.34 (s, 1H), 8.18 (s, 1H), 7.91-7.87 (m, 2H), 7.50 (t, J = 7.9 Hz, 1H), 7.33 (d, J = 7.8 Hz, 1H), 4.46 (q, J = 6.6 Hz, 1H), 4.37 (d, J = 10.4 Hz, 1H), 3.45 (s, 3H), 3.22-3.19 (m, 2H), 3.12-3.07 (m, 1H), 2.11 (dt, J = 3.6, 7.1 Hz, 1H), 1.90-1.77 (m, 4H), 1.76-1.69 (m, 1H), 1.44 (d, J = 6.9 Hz, 3H), 1.32-1.26 (m, 2H), 1.21- 1.14 (m, 2H), 0.61 (td, J = 7.5, 15.0 Hz, 6H). [α]_{D}²² −43.4° (c 0.1, MeOH) |
| D76 | 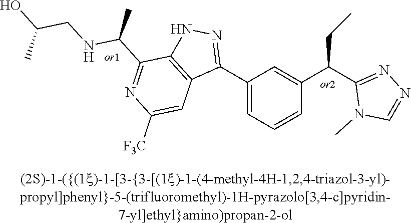 [SMILES: CC[C@H](c1cccc(-c2n[nH]c3c([C@H](C)NC[C@H](C)O)nc(C(F)(F)F)cc23)c1)c1nncn1C] | 488.4 | ¹H NMR (400 MHz, CD₃OD) δ 8.40 (s, 1H), 8.16 (s, 1H), 7.91 (br d, J = 7.7 Hz, 1H), 7.86 (s, 1H), 7.58 (t, J = 7.6 Hz, 1H), 7.38 (br d, J = 7.6 Hz, 1H), 4.45 (q, J = 6.7 Hz, 1H), 4.27 (t, J = 7.6 Hz, 1H), 3.97-3.86 (m, 1H), 3.56 (s, 3H), 2.69 (dd, J = 3.4, 11.9 Hz, 1H), 2.50-2.32 (m, 2H), 2.27-2.15 (m, 1H), 1.58 (d, J = 6.7 Hz, 3H), 1.13 (d, J = 6.4 Hz, 3H), 1.05 (t, J = 7.3 Hz, 3H). |
| D77 | 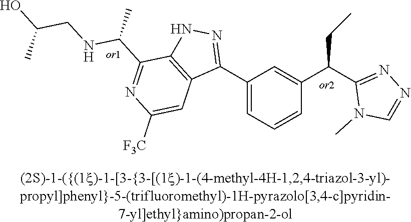 [SMILES: CC[C@H](c1cccc(-c2n[nH]c3c([C@@H](C)NC[C@H](C)O)nc(C(F)(F)F)cc23)c1)c1nncn1C] | 488.4 | ¹H NMR (400 MHz, CD₃OD) δ 8.39 (s, 1H), 8.16 (s, 1H), 7.91 (br d, J = 7.8 Hz, 1H), 7.86 (s, 1H), 7.57 (t, J = 7.7 Hz, 1H), 7.38 (br d, J = 7.6 Hz, 1H), 4.45 (q, J = 6.8 Hz, 1H), 4.27 (t, J = 7.6 Hz, 1H), 3.92-3.83 (m, 1H), 3.55 (s, 3H), 2.65 (dd, J = 7.8, 11.9 Hz, 1H), 2.51-2.38 (m, 2H), 2.27-2.15 (m, 1H), 1.58 (d, J = 6.7 Hz, 3H), 1.15 (d, J = 6.2 Hz, 3H), 1.05 (t, J = 7.3 Hz, 3H). |
| D78 | 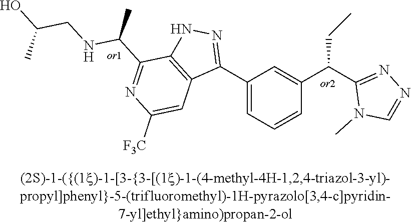 [SMILES: CC[C@@H](c1cccc(-c2n[nH]c3c([C@H](C)NC[C@H](C)O)nc(C(F)(F)F)cc23)c1)c1nncn1C] | 488.4 | ¹H NMR (400 MHz, CD₃OD) δ 8.40 (s, 1H), 8.17 (s, 1H), 7.91 (d, J = 7.9 Hz, 1H), 7.86 (s, 1H), 7.57 (t, J = 7.6 Hz, 1H), 7.38 (d, J = 7.9 Hz, 1H), 4.44 (q, J = 6.8 Hz, 1H), 4.27 (t, J = 7.6 Hz, 1H), 3.93-3.83 (m, 1H), 3.55 (s, 3H), 2.65 (dd, J = 7.8, 11.8 Hz, 1H), 2.49-2.38 (m, 2H), 2.26-2.15 (m, 1H), 1.57 (d, J = 6.8 Hz, 3H), 1.15 (d, J = 6.2 Hz, 3H), 1.05 (t, J = 7.3 Hz, 3H). |
| D79 | 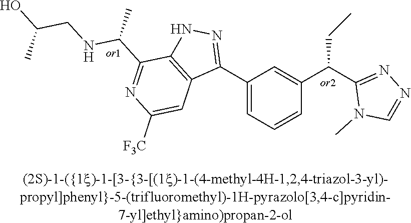 [SMILES: CC[C@@H](c1cccc(-c2n[nH]c3c([C@@H](C)NC[C@H](C)O)nc(C(F)(F)F)cc23)c1)c1nncn1C] | 488.4 | ¹H NMR (400 MHz, CD₃OD) δ 8.40 (s, 1H), 8.17 (s, 1H), 7.91 (d, J = 7.7 Hz, 1H), 7.86 (s, 1H), 7.57 (t, J = 7.7 Hz, 1H), 7.38 (br d, J = 7.9 Hz, 1H), 4.44 (q, J = 6.8 Hz, 1H), 4.27 (t, J = 7.6 Hz, 1H), 3.96- 3.87 (m, 1H), 3.55 (s, 3H), 2.69 (dd, J = 3.3, 11.9 Hz, 1H), 2.44 (td, J = 7.2, 13.9 Hz, 1H), 2.34 (dd, J = 8.5, 12.0 Hz, 1H), 2.26-2.15 (m, 1H), 1.57 (d, J = 6.8 Hz, 3H), 1.12 (d, J = 6.2 Hz, 3H), 1.05 |
| | | | (t, J = 7.4 Hz, 3H). |
| D80 | 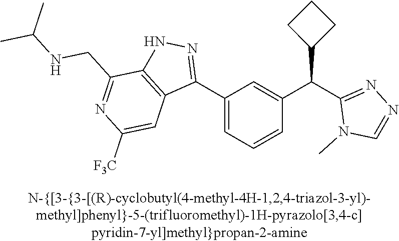 [SMILES: CC(C)NCc1nc(C(F)(F)F)cc2c(-c3cccc([C@H](c4nncn4C)C4CCC4)c3)n[nH]c12] | 484.0 | ¹H NMR (600 MHz, DMSO- d₆) δ 8.41-8.34 (m, 1H), 8.22 (s, 1H), 7.98-7.87 (m, 2H), 7.51 (t, J = 7.8 Hz, 1H), 7.32 (d, J = 7.7 Hz, 1H), 4.39 (d, J = 10.6 Hz, 1H), 4.29 (s, 2H), 3.48 (s, 3H), 3.27-3.25 (m, 1H), 2.81 (td, J = 6.2, 12.5 Hz, 1H), 2.40-2.33 (m, 1H), 2.20-2.07 (m, 1H), 1.91-1.81 (m, 4H), 1.08 (d, J = 6.2 Hz, 6H). [α]_{D}²² −55.3° (c 0.1, MeOH) |
| D81 | 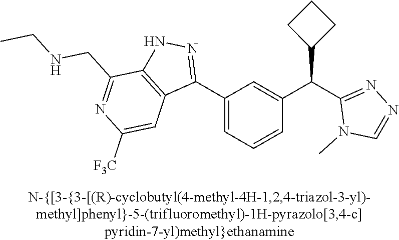 [SMILES: CCNCc1nc(C(F)(F)F)cc2c(-c3cccc([C@H](c4nncn4C)C4CCC4)c3)n[nH]c12] | 470.0 | ¹H NMR (600 MHz, DMSO- d₆) δ 8.32 (s, 1H), 8.22 (s, 1H), 7.95-7.83 (m, 2H), 7.49 (t, J = 7.7 Hz, 1H), 7.31 (d, J = 7.7 Hz, 1H), 4.35 (d, J = 10.5 Hz, 1H), 4.24 (s, 2H), 3.44 (s, 3H), 3.22-3.17 (m, 1H), 2.61 (q, J = 7.1 Hz, 2H), 2.18-2.04 (m, 1H), 1.86-1.66 (m, 5H), 1.05 (t, J = 7.1 Hz, 3H). |
| D82 | 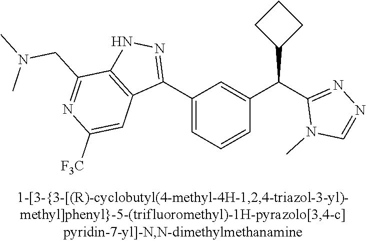 [SMILES: CN(C)Cc1nc(C(F)(F)F)cc2c(-c3cccc([C@H](c4nncn4C)C4CCC4)c3)n[nH]c12] | 470.0 | ¹H NMR (600 MHz, DMSO- d₆) δ 8.33 (s, 1H), 8.26 (s, 1H), 7.94-7.83 (m, 2H), 7.50 (t, J = 8.0 Hz, 1H), 7.34 (d, J = 7.9 Hz, 1H), 4.37 (d, J = 10.5 Hz, 1H), 3.95 (s, 2H), 3.45 (s, 3H), 3.24-3.19 (m, 1H), 2.27 (s, 6H), 2.18-2.00 (m, 1H), 1.92-1.67 (m, 5H). |
| D83 | 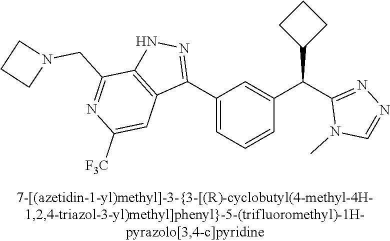 [SMILES: Cn1cnnc1[C@@H](c1cccc(-c2n[nH]c3c(CN4CCC4)nc(C(F)(F)F)cc23)c1)C1CCC1] | 482.0 | |
| D84 | 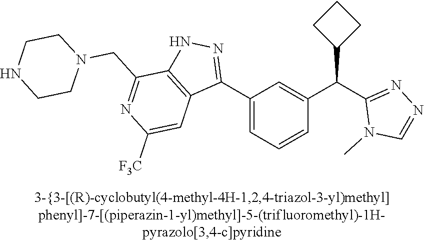 [SMILES: Cn1cnnc1[C@@H](c1cccc(-c2n[nH]c3c(CN4CCNCC4)nc(C(F)(F)F)cc23)c1)C1CCC1] | 511.0 | ¹H NMR (600 MHz, DMSO- d₆) δ 8.37-8.29 (m, 1H), 8.25 (s, 1H), 7.93-7.81 (m, 2H), 7.49 (t, J = 7.9 Hz, 1H), 7.32 (d, J = 7.7 Hz, 1H), 4.35 (d, J = 10.5 Hz, 1H), 3.96 (s, 2H), 3.44 (s, 3H), 3.23-3.18 (m, 1H), 2.75-2.70 (m, 4H), 2.45- 2.35 (m, 4H), 2.13-2.05 (m, 1H), 1.90-1.77 (m, 4H), 1.75- 1.67 (m, 1H). |
| D85 | 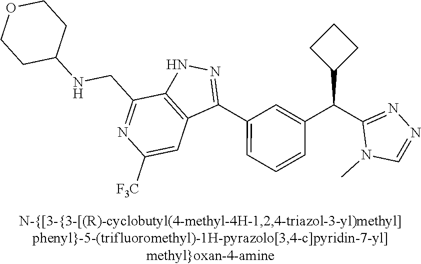 [SMILES: Cn1cnnc1[C@@H](c1cccc(-c2n[nH]c3c(CNC4CCOCC4)nc(C(F)(F)F)cc23)c1)C1CCC1] | 526.0 | ¹H NMR (600 MHz, DMSO- d₆) δ 8.32 (s, 1H), 8.21 (s, 1H), 7.97-7.80 (m, 2H), 7.48 (t, J = 7.6 Hz, 1H), 7.30 (d, J = 7.7 Hz, 1H), 4.35 (d, J = 10.6 Hz, 1H), 4.29 (s, 2H), 3.80 (td, J = 11.3, 3.4 Hz, 2H), 3.44 (s, 3H), 3.29-3.18 (m, 4H), 2.71-2.62 (m, 1H), 2.16-2.04 (m, 1H), 1.83-1.76 (m, 5H), 1.73-1.67 (m, 1H), 1.43-1.27 (m, 2H). |
| D86 | 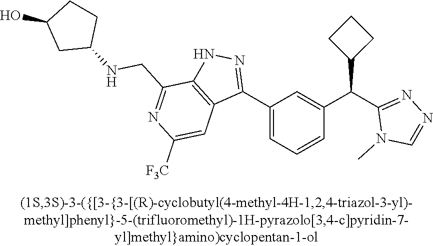 [SMILES: Cn1cnnc1[C@@H](c1cccc(-c2n[nH]c3c(CN[C@H]4CC[C@H](O)C4)nc(C(F)(F)F)cc23)c1)C1CCC1] | 526.1 | ¹H NMR (600 MHz, DMSO- d₆) δ 8.43-8.26 (m, 1H), 8.20 (s, 1H), 7.95-7.82 (m, 2H), 7.48 (t, J = 7.6 Hz, 1H), 7.29 (d, J = 7.7 Hz, 1H), 4.34 (d, J = 10.5 Hz, 1H), 4.21 (s, 2H), 4.17-4.08 (m, 1H), 3.26-3.19 (m, 2H), 3.16 (s, 4H), 2.16- 2.06 (m, 1H), 1.90-1.85 (m, 2H), 1.84-1.76 (m, 4H), 1.74- 1.62 (m, 2H), 1.52 (td, J = 13.1, 6.4 Hz, 1H), 1.41-1.27 (m, 2H). |
| D87 | 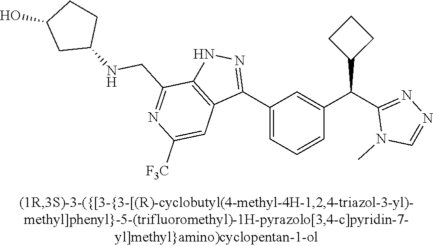 [SMILES: Cn1cnnc1[C@@H](c1cccc(-c2n[nH]c3c(CN[C@H]4CC[C@@H](O)C4)nc(C(F)(F)F)cc23)c1)C1CCC1] | 526.0 | ¹H NMR (600 MHz, DMSO- d₆) δ 8.34 (s, 1H), 8.24 (s, 1H), 7.95-7.85 (m, 2H), 7.51 (t, J = 7.9 Hz, 1H), 7.34 (d, J = 7.7 Hz, 1H), 4.37 (d, J = 10.5 Hz, 1H), 4.31-4.19 (m, 2H), 4.17- 4.09 (m, 1H), 3.46 (s, 3H), 3.26-3.18 (m, 1H), 3.15-3.11 (m, 1H), 2.18-2.06 (m, 1H), 1.89-1.68 (m, 8H), 1.66-1.60 (m, 2H), 1.59-1.48 (m, 1H). |
| D88 | 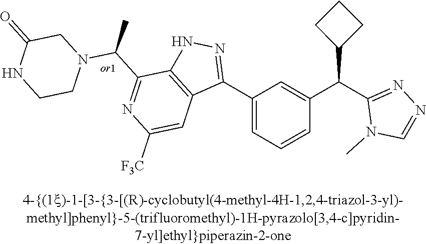 [SMILES: C[C@@H](c1nc(C(F)(F)F)cc2c(-c3cccc([C@H](c4nncn4C)C4CCC4)c3)n[nH]c12)N1CCNC(=O)C1] | 539.0 | ¹H NMR (600 MHz, DMSO- d₆) δ 8.33 (s, 1H), 8.26 (s, 1H), 7.95-7.84 (m, 2H), 7.71 (s, 1H), 7.50 (t, J = 7.9 Hz, 1H), 7.32 (br. d, J = 7.6 Hz, 1H), 4.42-4.25 (m, 2H), 3.44 (s, 3H), 3.10-3.01 (m, 3H), 2.70-2.53 (m, 3H), 2.20-2.08 (m, 1H), 1.87-1.64 (m, 5H), 1.48 (d, J = 6.8 Hz, 3H). [α]_{D}²² −55.4° (c 0.1, MeOH) |
| D89 | 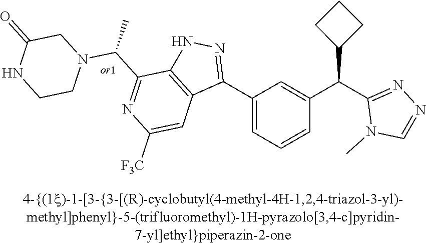 [SMILES: C[C@H](c1nc(C(F)(F)F)cc2c(-c3cccc([C@H](c4nncn4C)C4CCC4)c3)n[nH]c12)N1CCNC(=O)C1] | 539.0 | ¹H NMR (600 MHz, DMSO- d₆) δ 8.32 (s, 1H), 8.24 (s, 1H), 7.90-7.82 (m, 2H), 7.69 (s, 1H), 7.49 (t, J = 7.9 Hz, 1H), 7.30 (br. d, J = 7.5 Hz, 1H), 4.40-4.24 (m, 2H), 3.43 (s, 3H), 3.11-2.99 (m, 3H), 2.72-2.51 (m, 3H), 2.13-2.02 (m, 1H), 1.86-1.65 (m, 5H), 1.47 (d, J = 6.8 Hz, 3H). [α]_{D}²² −36.9° (c 0.1, MeOH) |
| D90 | 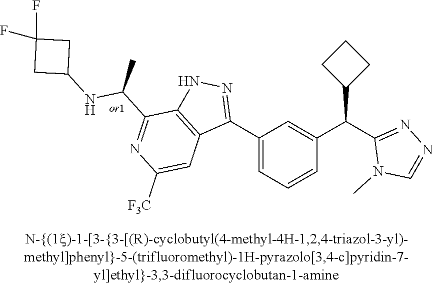 [SMILES: C[C@H](NC1CC(F)(F)C1)c1nc(C(F)(F)F)cc2c(-c3cccc([C@H](c4nncn4C)C4CCC4)c3)n[nH]c12] | 546.0 | ¹H NMR (600 MHz, DMSO- d₆) δ 8.36-8.31 (m, 1H), 8.24 (s, 1H), 7.92-7.85 (m, 2H), 7.51 (t, J = 7.9 Hz, 1H), 7.34 (d, J = 7.7 Hz, 1H), 4.43-4.32 (m, 2H), 3.45 (s, 3H), 3.25- 3.21 (m, 1H), 3.08-3.02 (m, 1H), 2.76-2.64 (m, 1H), 2.50- 2.37 (m, 3H), 2.31-2.17 (m, 1H), 2.16-2.05 (m, 1H), 1.90- 1.68 (m, 5H), 1.47 (d, J = 6.7 Hz, 3H). [α]_{D}²² −36.5° (c 0.1, MeOH) |
| D91 | 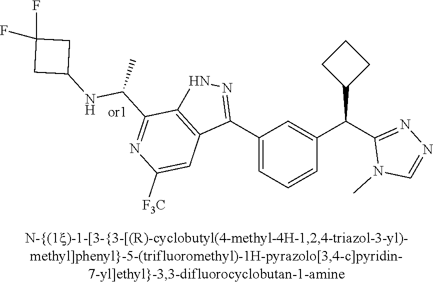 [SMILES: C[C@@H](NC1CC(F)(F)C1)c1nc(C(F)(F)F)cc2c(-c3cccc([C@H](c4nncn4C)C4CCC4)c3)n[nH]c12] | 546.0 | ¹H NMR (600 MHz, DMSO- d₆) δ 8.36-8.31 (m, 1H), 8.24 (s, 1H), 7.94-7.86 (m, 2H), 7.51 (t, J = 8.0 Hz, 1H), 7.34 (d, J = 7.8 Hz, 1H), 4.42-4.31 (m, 2H), 3.45 (s, 3H), 3.25- 3.21 (m, 1H), 3.07-3.02 (m, 1H), 2.75-2.64 (m, 1H), 2.50- 2.49 (m, 1H), 2.50-2.37 (m, 2H), 2.30-2.18 (m, 1H), 2.16- 2.07 (m, 1H), 1.89-1.70 (m, 5H), 1.47 (d, J = 6.8 Hz, 3H). [α]_{D}²² −78.7° (c 0.1, MeOH) |
| D92 | 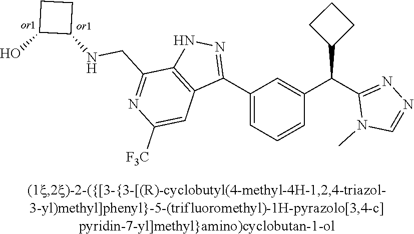 [SMILES: Cn1cnnc1[C@@H](c1cccc(-c2n[nH]c3c(CN[C@H]4CC[C@H]4O)nc(C(F)(F)F)cc23)c1)C1CCC1] | 512.2 | ¹H NMR (600 MHz, DMSO- d₆) δ 8.27 (s, 1H), 8.17 (s, 1H), 7.87-7.77 (m, 2H), 7.43 (t, J = 7.6 Hz, 1H), 7.26 (d, J = 7.6 Hz, 1H), 4.30 (d, J = 10.5 Hz, 1H), 4.23-4.13 (m, 2H), 4.12-4.06 (m, 1H), 3.38 (s, 3H), 3.25-3.22 (m, 2H), 3.18-3.12 (m, 1H), 2.10-2.00 (m, 1H), 1.96-1.86 (m, 1H), 1.83-1.65 (m, 6H), 1.64-1.53 (m, 1H). [α]_{D}²² −57.8° (c 0.1, MeOH) |
| D93 | 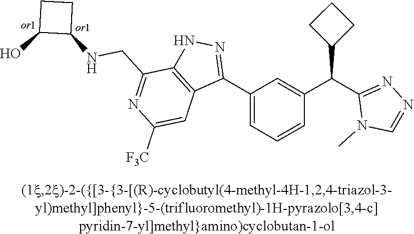 [SMILES: Cn1cnnc1[C@@H](c1cccc(-c2n[nH]c3c(CN[C@@H]4CC[C@@H]4O)nc(C(F)(F)F)cc23)c1)C1CCC1] | 512.2 | ¹H NMR (600 MHz, DMSO- d₆) δ 8.34 (s, 1H), 8.23 (s, 1H), 7.94-7.87 (m, 2H), 7.52 -7.46 (m, 1H), 7.32 (d, J = 7.6 Hz, 1H), 4.37 (d, J = 10.4 Hz, 1H), 4.28-4.20 (m, 2H), 4.20-4.15 (m, 1H), 3.46 (s, 3H), 3.41-3.37 (m, 1H), 3.33- 3.29 (m, 1H), 3.24-3.19 (m, 1H), 2.15-2.09 (m, 1H), 2.02- 1.94 (m, 1H), 1.89-1.80 (m, 4H), 1.78-1.72 (m, 2H), 1.71- 1.62 (m, 1H). [α]_{D}²² −43.8° (c 0.1, MeOH) |
| D94 | 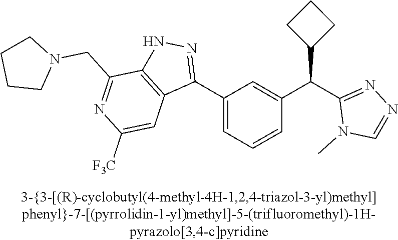 [SMILES: Cn1cnnc1[C@@H](c1cccc(-c2n[nH]c3c(CN4CCCC4)nc(C(F)(F)F)cc23)c1)C1CCC1] | 496.3 | ¹H NMR (400 MHz, CD₃OD) δ 8.26 (s, 1H), 8.08 (s, 1H), 7.81-7.76 (m, 1H), 7.75-7.72 (m, 1H), 7.44 (t, J = 7.8 Hz, 1H), 7.25 (d, J = 7.8 Hz, 1H), 4.23 (d, J = 10.9 Hz, 1H), 4.17 (s, 2H), 3.43 (s, 3H), 2.66 (br s, 4H), 2.28-2.11 (m, 1H), 1.88-1.76 (m, 10H). |
| D95 | 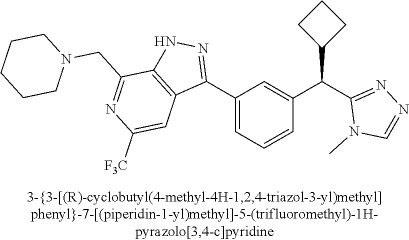 [SMILES: Cn1cnnc1[C@@H](c1cccc(-c2n[nH]c3c(CN4CCCCC4)nc(C(F)(F)F)cc23)c1)C1CCC1] | 510.3 | ¹H NMR (400 MHz, CD₃OD) δ 8.27 (s, 1H), 8.25-8.21 (m, 1H), 7.79 (br d, J = 7.5 Hz, 1H), 7.73 (br s, 1H), 7.47 (t, J = 7.7 Hz, 1H), 7.33 (br d, J = 7.7 Hz, 1H), 4.77 (br s, 2H), 4.24 (d, J = 10.9 Hz, 1H), 3.45 (s, 3H), 2.18 (dt, J = 10.8, 3.6 Hz, 1H), 2.07- 1.21 (m, 15H). |
| D96 | 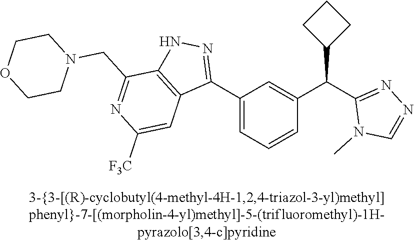 [SMILES: Cn1cnnc1[C@@H](c1cccc(-c2n[nH]c3c(CN4CCOCC4)nc(C(F)(F)F)cc23)c1)C1CCC1] | 512.3 | ¹H NMR (400 MHz, CD₃OD) δ 8.29-8.25 (m, 1H), 8.10-8.06 (m, 1H), 7.82-7.76 (m, 1H), 7.75-7.71 (m, 1H), 7.47-7.39 (m, 1H), 7.29-7.22 (m, 1H), 4.29-4.18 (m, 1H), 3.98 (s, 2H), 3.67-3.60 (m, 4H), 3.43 (s, 3H), 2.54-2.42 (m, 4H), 2.18 (br d, J = 7.3 Hz, 1H), 1.84 (br s, 4H), 1.78-1.65 (m, 2H). |
| D97 | 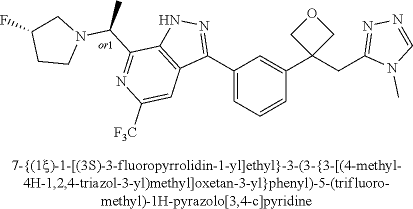 [SMILES: C[C@@H](c1nc(C(F)(F)F)cc2c(-c3cccc(C4(Cc5nncn5C)COC4)c3)n[nH]c12)N1CC[C@H](F)C1] | 530.0 | |
| D98 | 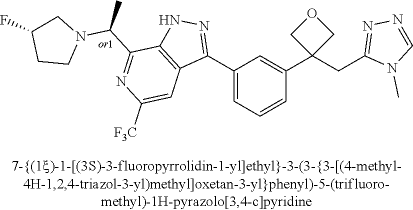 [SMILES: C[C@@H](c1nc(C(F)(F)F)cc2c(-c3cccc(C4(Cc5nncn5C)COC4)c3)n[nH]c12)N1CC[C@H](F)C1] | 530.0 | ¹H NMR (600 MHz, DMSO- ₆) δ 8.22 (s, 1H), 8.19 (s, 1H), 7.89 (d, J = 7.5 Hz, 1H), 7.59 (s, 1H), 7.49 (t, J = 7.6 Hz, 1H), 7.10 (d, J = 7.7 Hz, 1H), 5.35-5.13 (m, 1H), 5.00 (d, J = 6.0 Hz, 2H), 4.95 (dd, J = 1.7, 6.0 Hz, 2H), 4.19 (br. d, J = 6.7 Hz, 1H), 4.13-4.05 (m, 3H), 2.98 (s, 3H), 2.84- 2.80 (m, 1H), 2.75-2.70 (m, 1H), 2.66-2.59 (m, 1H), 2.21- 2.08 (m, 1H), 1.95-1.85 (m, |
| | | | 1H), 1.52 (d, J = 6.7 Hz, 3H). |

### P Example E1: 3-{3-[(R)-cyclobutyl(4-methyl-4H-1,2,4-triazol-3-yl)methyl]phenyl}-N,N-dimethyl-5-(trifluoromethyl)-1H-pyrazolo[3,4-b]pyridin-6-amine

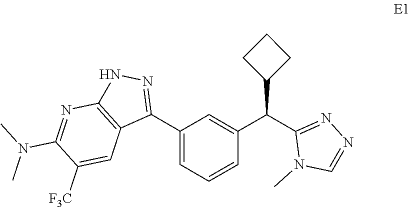 [SMILES: CN(C)c1nc2[nH]nc(-c3cccc([C@H](c4nncn4C)C4CCC4)c3)c2cc1C(F)(F)F]

Example E1 was prepared according to General Method E.

A mixture of 6-chloro-3-{3-[(R)-cyclobutyl(4-methyl-4H-1,2,4-triazol-3-yl)methyl]phenyl}-5-(trifluoromethyl)-1H-pyrazolo[3,4-b]pyridine (Intermediate 15) (60 mg, 0.13 mmol), dimethylamine hydrochloride (127 mg, 1.56 mmol) and Et₃N (204 mg, 2.01 mmol, 0.281 mL) in CH₃CN (1.0 mL, c=0.1 M) was heated at 100° C. overnight. The mixture was diluted with H₂O (5 mL) and extracted with DCM (2×5 mL). The combined organic layers were dried over Na₂SO₄ and concentrated. The crude product was purified by prep HPLC (Column: Phenemonex Luna Omega Polar C18 column, 150×21.2 mm, 5 um column; Mobile phase A: Water+10 mM Ammonium Acetate; Mobile phase B: Acetonitrile) to give 3-{3-[(R)-cyclobutyl(4-methyl-4H-1,2,4-triazol-3-yl)methyl]phenyl}-N,N-dimethyl-5-(trifluoromethyl)-1H-pyrazolo[3,4-b]pyridin-6-amine (Example E1) as a white solid (9.2 mg, 15%). ¹H NMR (600 MHz, DMSO-d₆) δ 8.48 (s, 1H), 8.31 (s, 1H), 7.82 (d, J=1.5 Hz, 1H), 7.80 (dd, J=1.1, 7.7 Hz, 1H), 7.42 (t, J=7.6 Hz, 1H), 7.23 (br. d, J=7.7 Hz, 1H), 4.31 (d, J=10.6 Hz, 1H), 3.42 (s, 3H), 3.20 (br. dd, J=1.7, 6.7 Hz, 1H), 2.93 (s, 6H), 2.16-2.05 (m, 1H), 1.88-1.75 (m, 4H), 1.74-1.66 (m, 1H); m/z (APCI+) for (C₂₃H₂₄F₃N₇) 456.1 (M+H).

Example E2 reported in Table 5 was synthesized with non-critical changes or substitutions to the exemplified procedures for Example E1 that one skilled in the art would be able to realize.

TABLE 5

| | | LCMS | |
|---|---|---|---|
| Example | | (m/z) | |
| Number | Structure and Name | (M + H)⁺ | ¹H NMR and Rotation |
| E2 | 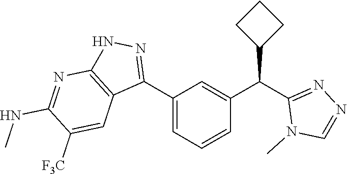 [SMILES: CNc1nc2[nH]nc(-c3cccc([C@H](c4nncn4C)C4CCC4)c3)c2cc1C(F)(F)F] | 442.1 | ¹H NMR (600 MHz, DMSO-d₆) δ 8.31 (s, 1H), 8.27 (s, 1H), 7.79 (s, 1H), 7.76 (d, J = 7.9 Hz, 1H), 7.42 (t, J = 7.6 Hz, 1H), 7.25 (d, J = 7.7 Hz, 1H), 6.77 − 6.73 (m, 1H), 4.31 (d, J = 10.6 Hz, 1H), 3.42 (s, 3H), 3.22 − 3.18 (m, 1H), 2.93 (d, J = 4.4 Hz, 3H), 2.15 − 2.07 (m, 1H), 1.87 − 1.75 (m, 4H), 1.71 (br. d, J = 2.4 Hz, 1H). [α]_{D}²² −68.7° (c 0.1, MeOH) |
| | 3-{3-[(R)-cyclobutyl(4-methyl- | | |
| | 4H-1,2,4-triazol-3- | | |
| | yl)methyl]phenyl}-N-methyl-5- | | |
| | (trifluoromethyl)-1H- | | |
| | pyrazolo[3,4-b]pyridin-6- | | |
| | amine | | |

### Example F1: N-{(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}cyclobutanamine

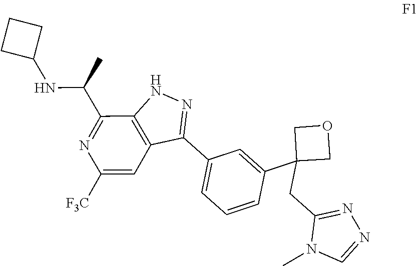

Example F1 was prepared according to Method F.

### Step 1: tert-butyl {(1S)-1-[3-bromo-5-(trifluoromethyl)-1H-pyrazolo[3,4-c]pyridin-7-yl]ethyl}-carbamate

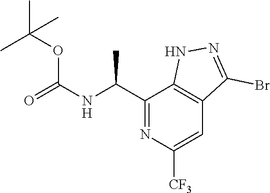 [SMILES: C[C@H](NC(=O)OC(C)(C)C)c1nc(C(F)(F)F)cc2c(Br)n[nH]c12]

To a 3-necked 1 L flask equipped with a condenser and an internal thermometer was added 1-[3-bromo-5-(trifluoromethyl)-1H-pyrazolo[3,4-c]pyridin-7-yl]ethan-1-ol (Intermediate 6) (22.7 g, 73 mmol, 1 equiv), (S,S)-2-methylpropane-2-sulfinamide (13.3 g, 110 mmol, 1.5 equiv), catalyst Ru-Macho (888 mg, 1.46 mmol, 0.02 equiv) and toluene (366 mL, 0.2 M). The flask was placed under nitrogen atmosphere. LHMDS (1 M in toluene, 110 mL, 110 mmol, 1.5 equiv) was added over 5 min to reach a final concentration of 0.15 M. The mixture was heated in an oil bath at 125° C. (bath temperature) overnight. Then, the volatiles were removed under reduced pressure. Sulfinamide deprotection: to the above residue was added methanol (300 mL) and aqueous HCl (6 M, 122 mL, 10 equiv) and stirred for 3 h. The volatiles were removed under reduced pressure.

Protecting with Boc group: to the above residue was added aqueous K₂CO₃ (2 M, 100 mL), to reach pH 8. Ethyl acetate (300 mL) was added followed by Boc anhydride (17.6 g, 80.5 mmol, 1.1 equiv). The mixture was stirred at room temperature overnight, then water (100 mL) was added, and the product was extracted with ethyl acetate (3×250 mL). The combined organic layer was dried over Na₂SO₄, filtered, and evaporated to give a solid gum (34.2 g). The crude material was purified on silica (20% ethyl acetate-80% heptane) to give tert-butyl {(1S)-1-[3-bromo-5-(trifluoromethyl)-1H-pyrazolo[3,4-c]pyridin-7-yl]ethyl}-carbamate as a light yellow solid (19.2 g, 64%, >99% ee). LCMS (ESI+) 409.0/411.0 (M+H). ¹H NMR (400 MHz, CDCl₃) δ=12.38 (br s, 1H), 7.89 (s, 1H), 5.46-5.31 (m, 1H), 5.13 (br d, J=8.1 Hz, 1H), 1.79 (d, J=6.8 Hz, 3H), 1.47 (s, 9H). ¹⁹F NMR (377 MHz, CHLOROFORM-d) δ=−66.46. [α]_{D}²²=−30.3° (° C. 0.5, MeOH).

The bis-Boc side product (5.1 g) which was isolated from the above column was treated with ammonia (7 N in methanol, 10 mL) for 1 hour at room temperature. Evaporation and purification as above gave an additional crop of the title product (3.4 g, 11%, total=75%).

### Step 2: tert-butyl {(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}carbamate

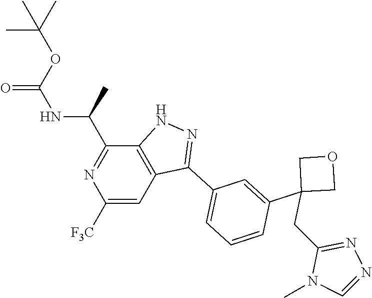

Aqueous 2M K₂CO₃ solution (4.39 g, 31.8 mmol, 15.9 mL) was added to a suspension of tert-butyl {(1S)-1-[3-bromo-5-(trifluoromethyl)-1H-pyrazolo[3,4-c]pyridin-7-yl]ethyl}carbamate (6.50 g, 15.9 mmol), 4-methyl-3-({3-[3-(4,4,5,5-tetramethyl-1,3,2-dioxaborolan-2-yl)phenyl]oxetan-3-yl}methyl)-4H-1,2,4-triazole (6.21 g, 17.5 mmol) and PdCl₂(DPPF)·CH₂Cl₂ (1.07 g, 1.31 mmol) in dioxane (53.0 mL, c=0.3 M). The resulting mixture was degassed three times. The reaction mixture was heated to reflux for 24 h. The mixture was cooled to RT, and 1/3 of solvent was removed under reduced pressure. The mixture was then filtered through Celite and washed with DCM. The filtrate was diluted with brine (100 mL), then organic layer was collected and aqueous was extracted with 10% iPrOH/DCM (100 mL×2). The combined organic layers were dried over Na₂SO₄ and concentrated. The crude product was purified by flash column chromatography (ISCO, 40 g SiO₂, solvent 0-10% MeOH/1:1 EtOAc:DCM to give tert-butyl {(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}carbamate as a brown color foam (7.8 g, 88%). LCMS (APCI+) 558.2 (M+H). ¹H NMR (CDCl₃, 400 MHz) δ 8.11 (s, 1H), 7.95 (br. s, 1H), 7.81 (d, 1H, J=7.8 Hz), 7.5-7.6 (m, 1H), 7.45 (t, 1H, J=7.7 Hz), 6.87 (d, 1H, J=7.3 Hz), 5.44 (br. s, 1H), 5.1-5.2 (m, 4H), 3.64 (s, 2H), 2.85 (s, 3H), 1.78 (d, 3H, J=6.5 Hz), 1.49 (br. s, 9H).

### Step 3: (1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethan-1-amine TFA salt

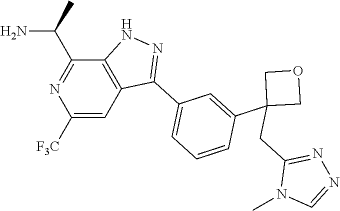

A solution of tert-butyl {(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}carbamate (7.80 g, 14.0 mmol) in DCM (70 mL, c=0.2 M) was added trifluoracetic acid (18.8 mL, 296 mmol) at room temperature and the mixture was stirred at room temperature for 2 h. Toluene (10 mL) was added to the reaction mixture and the volatile was removed under reduced pressure. The crude residue was azeotroped with toluene three times to give a dark brown-reddish color foam which was dried on high vacuum overnight to give the title compound (11.2 mg, 100%) as reddish color foam which was carried forward without further purification. LCMS (APCI+) 458.1 (M+H).

### Step 4: N-{(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}cyclobutanamine

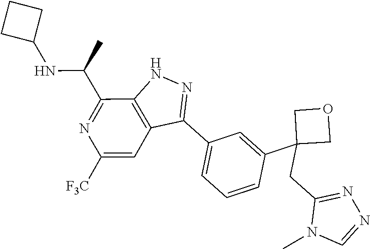

Et₃N (6.19 mL, 44.4 mmol) was added to a solution of (1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethan-1-amine TFA salt (11.2 g, 14.0 mmol) in EtOH (70.0 mL, c=0.2 M), then added anhydrous MgSO₄ (8.42 g, 70.0 mmol) and cyclobutanone (2.06 mL, 28.0 mmol). The resulting mixture was stirred at RT for 18 h. Then, AcOH (0.80 mL, 14.0 mmol) and NaBH₃CN (4.63 g, 70.0 mmol) were added. The resulting reaction mixture was stirred at RT for 2 h. The reaction was quenched with NH₄OAc (32.4 g, 420 mmol) in H₂O (100 mL) and the mixture was heated at 50° C. for 1 h. The mixture was filtered through Celite and washed with DCM. The filtrate was concentrated under reduced pressure to remove half of EtOH, then diluted with brine (100 mL) and extracted with DCM (80 mL). The organic layer was collected and the aqueous was extracted with 10% iPrOH/DCM (2×80 mL). The combined organic layers were dried over Na₂SO₄ and concentrated. The crude product was purified by flash column chromatography (ISCO, 40 g SiO₂, solvent 0-10% MeOH/DCM to 10% 7N NH₃ in MeOH/DCM to give N-{(1S)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}cyclobutanamine (Example F1) as a pale-yellow solid (6.63 g, 88%). The product was treated with 7N NH₃ in MeOH for 1 h to further breakdown the boron-product complex. The volatiles were removed under reduced pressure to give a pale-yellow solid.

Crystallization: several batches of the title product were combined (total ˜7.2 g solid). A premixed solution of 10% methanol in water (40 mL) was added. The mixture was stirred and heated in a pre-heated oil bath at 60° C. for 1 h. If there was a paste, a spatula was used to scratch the flask wall to initiate solidification. The mixture was then stirred at room temperature for 4-18 h. The solid was filtered, washed with water (15 mL) and dried to give the title product (7.04 g, 98% recovery, >99% ee). LCMS (APCI+) 512.3 (M+H). ¹H NMR (400 MHz, DMSO-d₆) δ 8.18 (d, J=1.5 Hz, 2H), 7.88 (d, J=7.9 Hz, 1H), 7.60 (s, 1H), 7.48 (t, J=7.8 Hz, 1H), 7.08 (d, J=8.0 Hz, 1H), 5.03-4.97 (m, 2H), 4.96-4.91 (m, 2H), 4.33 (q, J=6.7 Hz, 1H), 3.58 (s, 2H), 3.05 (quin, J=7.5 Hz, 1H), 2.98 (s, 3H), 2.09-1.96 (m, 1H), 1.72 (s, 1H), 1.68-1.57 (m, 1H), 1.55-1.46 (m, 2H), 1.44 (d, J=6.8 Hz, 3H), 1.39 (br s, 1H). ¹⁹F NMR (376 MHz, DMSO-d₆) δ −64.2. [α]_{D}²² −24.8° (c 0.4, MeOH). Small molecule X-ray confirmed the absolute stereocenters of the title product.

### Example G1: (1S)—N-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}-3,3-difluorocyclopentan-1-amine; and

### Example G2: (1S)—N-{[3-{3-[(S)-cyclobutyl(4-methyl-4H-1,2,4-triazol-3-yl)methyl]phenyl}-5-(trifluoromethyl)-1H-pyrazolo[3,4-c]pyridin-7-yl]methyl}-3,3-difluorocyclopentan-1-amine

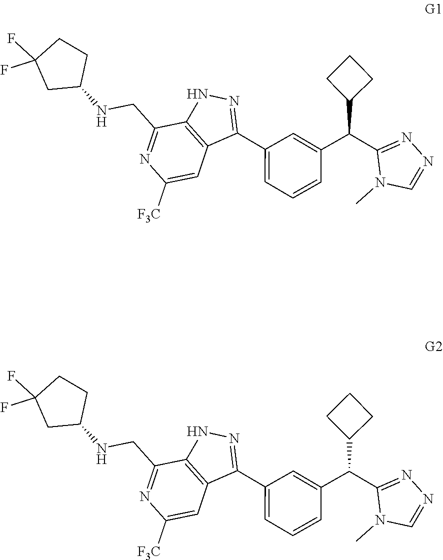 [SMILES: Cn1cnnc1[C@@H](c1cccc(-c2n[nH]c3c(CN[C@H]4CCC(F)(F)C4)nc(C(F)(F)F)cc23)c1)C1CCC1 ; Cn1cnnc1[C@H](c1cccc(-c2n[nH]c3c(CN[C@H]4CCC(F)(F)C4)nc(C(F)(F)F)cc23)c1)C1CCC1]

Examples G1 and G2 were prepared according to Method G.

### Step 1

A mixture of (S)—N-((3-bromo-5-(trifluoromethyl)-1H-pyrazolo[3,4-c]pyridin-7-yl)methyl)-3,3-difluorocyclopentan-1-amine (Intermediate 16) (69 mg, 0.17 mmol), Boc₂O (58.3 mg, 0.259 mmol), triethylamine (52.5 mg, 0.519 mmol, 0.0723 mL) and DMAP (2.51 mg, 0.0205 mmol) in DCM (3.0 mL) was stirred at room temperature overnight. The mixture was quenched with brine, extracted with DCM (2×5 mL). The combined organic layers were dried over Na₂SO₄ and concentrated under vacuum. The crude mixture was used in the next step without further purification. The crude mixture was dissolved in 1,4-dioxane (2.0 mL) and H₂O (0.5 mL). To the solution was added 3-{cyclobutyl[3-(4,4,5,5-tetramethyl-1,3,2-dioxaborolan-2-yl)phenyl]-methyl}-4-methyl-4H-1,2,4-triazole (Intermediate 1) (79.6 mg, 0.225 mmol), Cs₂CO₃ (147 mg, 0.451 mmol) and Pd(dppf)Cl₂·DCM (12.3 mg, 0.015 mmol). The mixture was degassed with N₂ three times and then was heated at 100° C. for 2 h. The mixture was cooled to RT and an additional aliquot of 3-{cyclobutyl[3-(4,4,5,5-tetramethyl-1,3,2-dioxaborolan-2-yl)phenyl]methyl}-4-methyl-4H-1,2,4-triazole (Intermediate 1) (50 mg, 0.14 mmol) and Pd(dppf)Cl₂·DCM (16 mg, 0.02 mmol) was added and heated at 100° C. for 1 hr. The mixture was cooled to room temperature, diluted with brine (5 mL) and extracted with DCM (2×5 mL). The combined organic layers were dried over Na₂SO₄, filtered and concentrated. The crude product was purified by flash column chromatography (ISCO, 12 g SiO₂, solvent 0-10% MeOH/1:1 EtOAc/DCM) to give a pale brow color gum as product (79 mg, 93%). m/z (APCI+) for (C₃₂H₃₆F₅N₇₀₂) 646.0 (M+H). 4N HCl in dioxane (0.563 mL, 2.25 mmol) was added to the aforementioned product (79 mg, 0.122 mmol) in DCM (2 mL). The mixture was stirred at RT for 1 hr. The volatile was removed under reduced pressure. The crude product was purified by Chiral SFC (Column: ES Industries ChromegaChiral CCOF₄, 250×20 mm, 5 um; Temperature: 35.0° C.; Pressure: 120.0 bar; Flow Rate: 100.000 mL/min; Eluted with 13.0% MeOH+10 mM NH₃ in CO₂; Mobile Phase Composition: CO₂; Solvent: MeOH+10 mM NH₃). (1S)—N-{[3-{3-[(S)-cyclobutyl(4-methyl-4H-1,2,4-triazol-3-yl)methyl]phenyl}-5-(trifluoromethyl)-1H-pyrazolo[3,4-c]pyridin-7-yl]methyl}-3,3-difluorocyclopentan-1-amine, the first eluting peak, obtained as a white solid (3.9 mg, 3%). ¹H NMR (DMSO-d₆, 600 MHz) δ 8.34 (s, 1H), 8.25 (s, 1H), 7.89-7.90 (m, 2H), 7.51 (br. t, 1H, J=7.9 Hz), 7.34 (br. d, 1H, J=7.5 Hz), 4.37 (br. d, 1H, J=10.5 Hz), 4.25 (s, 2H), 3.46 (s, 3H, overlap with H₂O), 3.29-3.30 (m, 1H, overlap with H₂O), 3.21-3.24 (m, 1H), 2.28-2.41 (m, 1H), 2.17-2.25 (m, 1H), 2.10-2.12 (m, 1H), 1.94-2.03 (m, 3H), 1.80-1.88 (m, 4H), 1.71-1.76 (m, 1H), 1.59-1.68 (m, 1H); m/z (APCI+) for (C₂₇H₂₈F₅N₇) 546.1 (M+H); [α]_{D}²²=+57.3° (c 0.1, MeOH). (1S)—N-{[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]methyl}-3,3-difluorocyclopentan-1-amine, the second eluting peak, obtained as a white solid (4.4 mg, 4%). ¹H NMR (DMSO-d₆, 600 MHz) δ 8.34 (s, 1H), 8.25 (s, 1H), 7.9-7.9 (m, 2H), 7.51 (t, 1H, J=7.9 Hz), 7.34 (br. d, 1H, J=7.5 Hz), 4.37 (d, 1H, J=10.5 Hz), 4.25 (s, 2H), 3.46 (s, 3H, overlap with H₂O), 3.27-3.29 (m, 1H, overlap with H₂O), 3.19-3.24 (m, 1H, overlap with H₂O), 2.28-2.40 (m, 1H), 2.16-2.25 (m, 1H), 2.09-2.13 (m, 1H), 1.94-2.05 (m, 3H), 1.79-1.88 (m, 4H), 1.71-1.77 (m, 1H), 1.60-1.68 (m, 1H); m/z (APCI+) for (C₂₇H₂₈F₅N₇) 546.1 (M+H); [α]_{D}²²=−59.9° (c 0.1, MeOH).

### Example H1: (2S)-1-({(1)-1-[3-{3-[(R)-cyclobutyl(4-methyl-4H-1,2,4-triazol-3-yl)methyl]phenyl}-5-cyclopropyl-1H-pyrazolo[3,4-c]pyridin-7-yl]ethyl}amino)propan-2-ol; and

### Example H2: (2S)-1-({(1)-1-[3-{3-[(R)-cyclobutyl(4-methyl-4H-1,2,4-triazol-3-yl)methyl]phenyl}-5-cyclopropyl-1H-pyrazolo[3,4-c]pyridin-7-yl]ethyl}amino)propan-2-ol

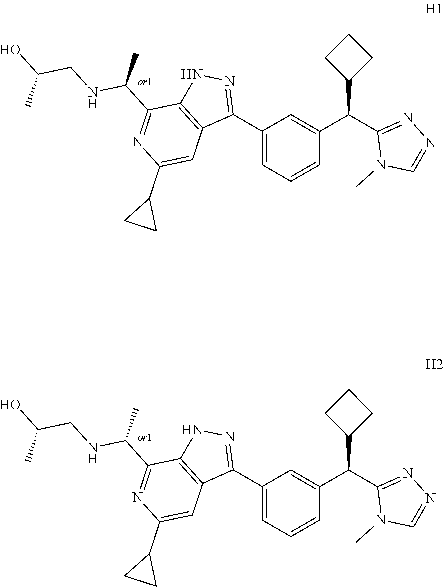 [SMILES: C[C@H](O)CN[C@@H](C)c1nc(C2CC2)cc2c(-c3cccc([C@H](c4nncn4C)C4CCC4)c3)n[nH]c12 ; C[C@H](O)CN[C@H](C)c1nc(C2CC2)cc2c(-c3cccc([C@H](c4nncn4C)C4CCC4)c3)n[nH]c12]

Examples H1 and H2 were prepared according to General Method H.

A suspension of 1-(3-{3-[(R)-cyclobutyl(4-methyl-4H-1,2,4-triazol-3-yl)methyl]phenyl}-5-cyclopropyl-1H-pyrazolo[3,4-c]pyridin-7-yl)ethan-1-one (Intermediate 20) (200 mg, 0.469 mmol), (S)-(+)-1-amino-2-propanol (352 mg, 4.69 mmol), and Ti(OEt)₄ (535 mg, 2.34 mmol) in EtOH (5 mL) was stirred at 70° C. for 1.5 h, then NaBH₃CN (147 mg, 2.34 mmol) was added. The resulting mixture was stirred at 70° C. for 16 h. Then, additional (S)-(+)-1-amino-2-propanol (352 mg, 4.69 mmol), and Ti(OEt)₄ (535 mg, 2.34 mmol) in EtOH (5 mL) was added and stirred at 70° C. for 1.5 h. Additional NaBH₃CN (147 mg, 2.34 mmol) was added and the resulting mixture was stirred at 70° C. for 16 h. The mixture was diluted with EtOAc (15 mL) and quenched with aqueous sodium bicarbonate (10 mL) and 15% NaOH solution (5 mL). The mixture was stirred at RT for 30 min. The layers were separated, and the organic layer was dried over Na₂SO₄, filtered, and concentrated under reduced pressure. The crude residue was purified by flash column chromatography (Isco, SiO₂, 0-10% DCM/MeOH) to give the crude residue (75 mg). The crude residue was further purified via preparative SFC with Daicel ChiralPak IG column (250×30 mm, 10 μm particle size), Flow Rate: 80.000 mL/min, eluted with 55% EtOH+0.1% NH₃H₂O in CO₂). (2S)-1-({(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol (Example H1) was obtained as the first eluting peak as a white solid (27 mg, 8%). LCMS (APCI+) 486.5 (M+H)⁺. ¹H NMR (400 MHz, DMSO-d₆) δ 8.34 (s, 1H), 7.83 (brd, J=7.8 Hz, 1H), 7.79 (s, 1H), 7.56 (s, 1H), 7.47 (t, J=7.7 Hz, 1H), 7.28 (br d, J=7.8 Hz, 1H), 4.30 (br d, J=10.6 Hz, 1H), 4.18 (br d, J=6.6 Hz, 2H), 3.74-3.63 (m, 1H), 3.42 (s, 3H), 3.26-3.13 (m, 1H), 2.44-2.37 (m, 1H), 2.22-2.06 (m, 3H), 1.88-1.60 (m, 6H), 1.34 (br d, J=6.6 Hz, 3H), 0.97-0.88 (m, 7H). 2S)-1-({(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}amino)propan-2-ol (Example H2) was obtained as the second eluting peak as a white solid (16.8 mg, 7%). LCMS (APCI+) 486.5 (M+H)⁺. ¹H NMR (400 MHz, DMSO-d₆) δ 8.35 (s, 1H), 7.84 (d, J=1.0 Hz, 2H), 7.59 (s, 1H), 7.47 (t, J=7.5 Hz, 1H), 7.28 (br d, J=7.6 Hz, 1H), 4.32 (br d, J=10.5 Hz, 1H), 4.16 (q, J=6.5 Hz, 1H), 3.43 (s, 3H), 3.21 (br d, J=9.7 Hz, 1H), 2.40 (br dd, J=7.0, 11.4 Hz, 2H), 2.26-2.16 (m, 3H), 2.14-2.06 (m, 1H), 1.88-1.67 (m, 6H), 1.37 (d, J=6.7 Hz, 3H), 0.99 (d, J=6.1 Hz, 3H), 0.92 (br d, J=8.3 Hz, 4H).

### Example H3: N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine; and

### Example H4: N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine

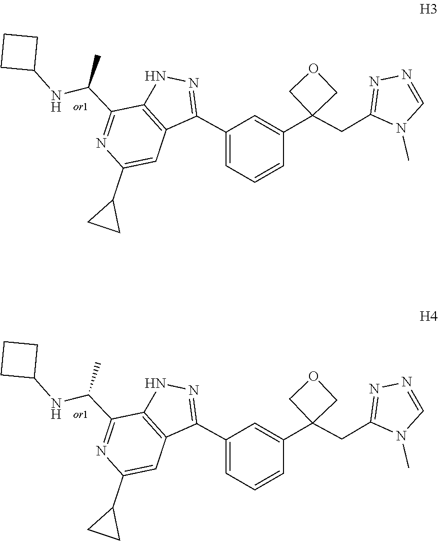 [SMILES: C[C@H](NC1CCC1)c1nc(C2CC2)cc2c(-c3cccc(C4(Cc5nncn5C)COC4)c3)n[nH]c12 ; C[C@@H](NC1CCC1)c1nc(C2CC2)cc2c(-c3cccc(C4(Cc5nncn5C)COC4)c3)n[nH]c12]

Examples H3 and H4 were prepared according to General Method H.

A suspension of 1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-one (Intermediate 21) (100 mg, 0.469 mmol), cyclobutylamine (166 mg, 2.33 mmol), and Ti(OEt)₄ (266 mg, 1.17 mmol) in EtOH (3 mL) was stirred at 70° C. for 1.5 h, then NaBH₃CN (73.3 mg, 1.17 mmol) was added. The resulting mixture was stirred at 70° C. for 16 h. The mixture was diluted with EtOAc (10 mL) and quenched with aqueous sodium bicarbonate (5 mL) and 15% NaOH solution (2 mL). The mixture was stirred at 25° C. for 30 min. The layers were separated, and the organic layer was dried over Na₂SO₄, filtered, and concentrated under reduced pressure. The crude residue was purified by flash column chromatography (Isco, SiO₂, 0-10% DCM/MeOH) to give the crude residue (85 mg). The crude residue was further purified via preparative SFC with Daicel ChiralPak AD column (250×30 mm, 10 μm particle size), Flow Rate: 80.000 mL/min, eluted with 45% IPA+0.1% NH₃H₂O in CO₂). N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine (Example H3) was obtained as the first eluting peak as a white solid (22.2 mg, 20%). LCMS (APCI+) 484.4 (M+H)⁺. ¹H NMR (400 MHz, DMSO-d₆) δ 8.23 (s, 1H), 7.84 (d, J=8.0 Hz, 1H), 7.50 (s, 1H), 7.49 (s, 1H), 7.46 (s, 1H), 7.05 (br d, J=8.1 Hz, 1H), 5.01 (d, J=6.0 Hz, 2H), 4.97-4.94 (m, 2H), 4.22 (br d, J=6.2 Hz, 1H), 3.59 (s, 2H), 3.09-3.00 (m, 1H), 2.94 (s, 3H), 2.31-2.27 (m, 1H), 2.01 (br s, 1H), 1.76-1.57 (m, 3H), 1.54-1.40 (m, 3H), 1.35 (d, J=6.8 Hz, 3H), 0.99-0.90 (m, 4H). N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine (Example H4) was obtained as the second eluting peak as white solid (22.7 mg, 20%). LCMS (APCI+) 484.4 (M+H)⁺. ¹H NMR (400 MHz, DMSO-d₆) δ 8.23 (s, 1H), 7.84 (d, J=8.0 Hz, 1H), 7.49 (s, 1H), 7.49-7.43 (m, 2H), 7.04 (d, J=8.1 Hz, 1H), 5.01 (d, J=1.0 Hz, 2H), 4.95 (d, J=6.1 Hz, 2H), 4.18 (q, J=6.6 Hz, 1H), 3.59 (s, 2H), 3.05-2.96 (m, 1H), 2.94 (s, 3H), 2.32-2.27 (m, 1H), 2.05-1.95 (m, 1H), 1.75-1.54 (m, 3H), 1.52-1.37 (m, 3H), 1.34 (d, J=6.7 Hz, 3H), 0.99-0.90 (m, 4H).

### Example H5: (1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol; and

### Example H6: (1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol

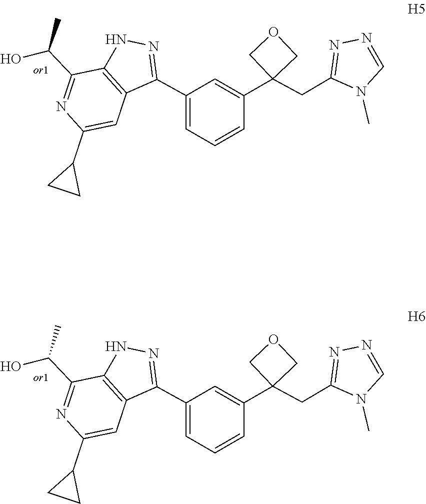 [SMILES: C[C@H](O)c1nc(C2CC2)cc2c(-c3cccc(C4(Cc5nncn5C)COC4)c3)n[nH]c12 ; C[C@@H](O)c1nc(C2CC2)cc2c(-c3cccc(C4(Cc5nncn5C)COC4)c3)n[nH]c12]

Examples H5 and H6 were prepared according to General Method H (Intermediates MH-4).

Intermediate 21a (107 mg, 0.249 mmol) was purified via preparative chiral SFC (Daicel ChiralPak AD column (250×30 mm, 10 μm particle size), Flow Rate: 80.000 mL/min, eluted with 50% IPA+0.1% NH₃H₂O in CO₂). (1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol (Example H5) was obtained as the first eluting peak as a white solid (40.8 mg, 38%). LCMS (ESI+) 431.1 (M+H)⁺. ¹H NMR (400 MHz, DMSO-d₆) δ 13.32 (brs, 1H), 8.23 (s, 1H), 7.84 (d, J=7.7 Hz, 1H), 7.54 (s, 1H), 7.51-7.43 (m, 2H), 7.05 (d, J=8.1 Hz, 1H), 5.67 (br d, J=4.4 Hz, 1H), 5.05 (br d, J=5.7 Hz, 1H), 5.02 (br d, J=6.2 Hz, 2H), 4.98-4.94 (m, 2H), 3.59 (s, 2H), 2.94 (s, 3H), 2.32-2.27 (m, 1H), 1.49 (d, J=6.6 Hz, 3H), 0.98-0.89 (m, 4H). (1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethan-1-ol (Example H6) was obtained as the second eluting peak as a white solid (42.4 mg, 40%). LCMS (ESI+) 431.1 (M+H)⁺. ¹H NMR (400 MHz, DMSO-d₆) δ 13.32 (br s, 1H), 8.22 (s, 1H), 7.84 (d, J=7.9 Hz, 1H), 7.54 (s, 1H), 7.50-7.44 (m, 2H), 7.05 (d, J=8.1 Hz, 1H), 5.67 (d, J=4.4 Hz, 1H), 5.08-5.04 (m, 1H), 5.03-5.00 (m, 2H), 4.96 (d, J=1.0 Hz, 2H), 3.59 (s, 2H), 2.94 (s, 3H), 2.32-2.27 (m, 1H), 1.48 (d, J=6.4 Hz, 3H), 0.97-0.90 (m, 4H).

### Example 11: N-{(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}-2-hydroxyacetamide; and

### Example 12: N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine

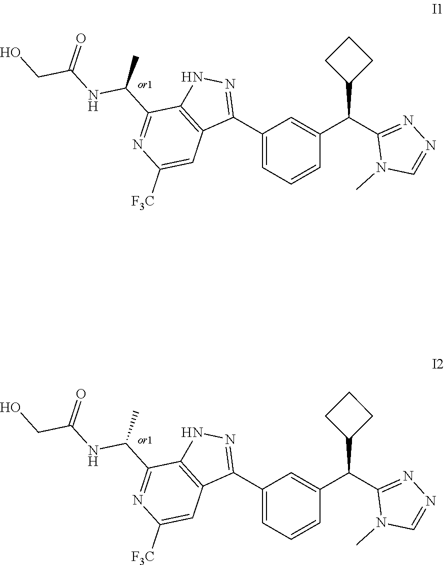 [SMILES: C[C@H](NC(=O)CO)c1nc(C(F)(F)F)cc2c(-c3cccc([C@H](c4nncn4C)C4CCC4)c3)n[nH]c12 ; C[C@@H](NC(=O)CO)c1nc(C(F)(F)F)cc2c(-c3cccc([C@H](c4nncn4C)C4CCC4)c3)n[nH]c12]

Examples 11 and 12 were prepared according to General Method I.

### Step 1: 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-amine

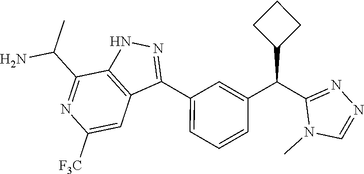 [SMILES: CC(N)c1nc(C(F)(F)F)cc2c(-c3cccc([C@H](c4nncn4C)C4CCC4)c3)n[nH]c12]

To a suspension of 7-(1-bromoethyl)-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 12) (7.47 g, 2.9 mmol) in CH₃CN (40 mL) was added i-Pr₂NEt (1.86 g, 14.4 mmol) and NH₃·H₂O (10 mL). The mixture was stirred at 25° C. for 2 h before it was diluted with water (20 mL), EtOAc (50 mL) and 1N HCl (100 mL to reach pH<7). The layers were separated and the organic layer was extracted with water (3×100 mL). Then the combined aqueous layers were adjusted to pH>7 using aqueous sodium bicarbonate and extracted with EtOAc (3×100 mL). The combined organic layers were dried over Na₂SO₄, filtered, and concentrated under reduced pressure. The residue was purified by flash column chromatography (Isco, SiO₂, 0-15% DCM/MeOH) to afford 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-amine (580 mg, 44%) as a grey solid. LCMS (ESI+) 456.3 (M+H)⁺. ¹H NMR (400 MHz, DMSO-d₆) δ 8.34 (s, 1H), 8.22 (s, 1H), 7.92-7.86 (m, 2H), 7.49 (t, J=7.9 Hz, 1H), 7.32 (br d, J=7.5 Hz, 1H), 4.61 (q, J=6.6 Hz, 1H), 4.37 (br d, J=10.6 Hz, 2H), 3.45 (s, 3H), 3.21 (br d, J=10.1 Hz, 1H), 2.16-2.04 (m, 1H), 1.91-1.68 (m, 6H), 1.48 (d, J=6.6 Hz, 3H); ¹⁹F NMR (376 MHz, DMSO-d₆) δ −64.22 (br s, 3F).

### Step 2: N-{(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}-2-hydroxyacetamide and N-{(1ξ)-1-[5-cyclopropyl-3-(3-{3-[(4-methyl-4H-1,2,4-triazol-3-yl)methyl]oxetan-3-yl}phenyl)-1H-pyrazolo[3,4-c]pyridin-7-yl]ethyl}cyclobutanamine

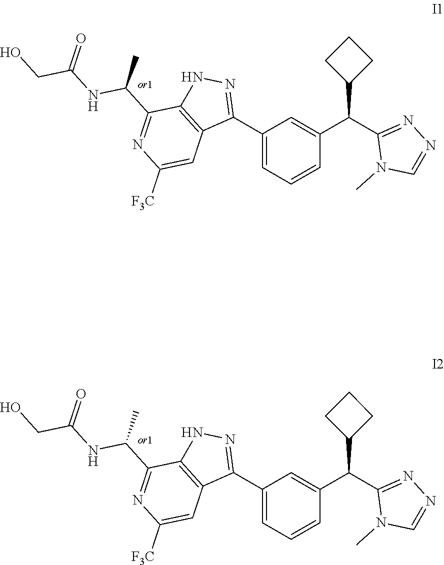 [SMILES: C[C@H](NC(=O)CO)c1nc(C(F)(F)F)cc2c(-c3cccc([C@H](c4nncn4C)C4CCC4)c3)n[nH]c12 ; C[C@@H](NC(=O)CO)c1nc(C(F)(F)F)cc2c(-c3cccc([C@H](c4nncn4C)C4CCC4)c3)n[nH]c12]

A solution of 1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethan-1-amine (170 mg, 0.373 mmol), glycolic acid (28.4 mg, 0.373 mmol), HATU (213 mg, 0.560 mmol), and i-Pr₂NEt (0.325 mL, 1.87 mmol) in DMF (6 mL) was stirred at 20° C. After 3 h, the mixture was diluted with water (10 mL) and extracted with EtOAc (8×5 mL). The combined organic layers were washed with brine, dried over Na₂SO₄, filtered, and concentrated under reduced pressure. Purification via flash column chromatography (Isco, SiO₂, 0-15% DCM/MeOH) gave the crude residue. The crude residue was further purified via preparative SFC with Daicel ChiralPak AS column (250×30 mm, 10 μm particle size), Flow Rate: 100.000 mL/min, eluted with 30% EtOH+0.1% NH₃H₂O in CO₂). (N-{(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}-2-hydroxyacetamide (Example 11) was obtained as the first eluting peak as a white solid (20.2 mg, 11%). LCMS (ESI+) 514.3 (M+H)⁺. ¹H NMR (400 MHz, CD₃OD) δ 8.38 (s, 1H), 8.19 (s, 1H), 7.88 (d, J=7.7 Hz, 1H), 7.84 (s, 1H), 7.54 (t, J=7.7 Hz, 1H), 7.35 (d, J=7.9 Hz, 1H), 5.71 (q, J=6.8 Hz, 1H), 4.33 (d, J=10.8 Hz, 1H), 4.16-3.97 (m, 2H), 3.53 (s, 3H), 3.35 (br s, 1H), 2.34-2.21 (m, 1H), 1.99-1.87 (m, 4H), 1.84-1.74 (m, 1H), 1.65 (d, J=6.8 Hz, 3H). ¹⁹F NMR (376 MHz, CD₃OD) δ −67.5. (N-{(1ξ)-1-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]ethyl}-2-hydroxyacetamide (Example 12) was obtained as the second eluting peak as white solid (23.4 mg, 12%). LCMS (APCI+) 514.3 (M+H)⁺. ¹H NMR (400 MHz, CD₃OD) δ 8.38 (s, 1H), 8.19 (s, 1H), 7.88 (br d, J=7.7 Hz, 1H), 7.85 (s, 1H), 7.54 (t, J=7.7 Hz, 1H), 7.35 (br d, J=7.7 Hz, 1H), 5.70 (q, J=6.7 Hz, 1H), 4.33 (d, J=11.0 Hz, 1H), 4.12-3.98 (m, 2H), 3.53 (s, 3H), 3.35 (br s, 1H), 2.35-2.23 (m, 1H), 2.00-1.87 (m, 4H), 1.85-1.74 (m, 1H), 1.66 (d, J=6.8 Hz, 3H). ¹⁹F NMR (376 MHz, CD₃OD) δ −67.5.

Examples 13-16 reported in Table 6 were synthesized with non-critical changes or substitutions to the exemplified procedures for Examples 11 and 12 that one skilled in the art would be able to realize.

TABLE 6

| | | LCMS | |
|---|---|---|---|
| Example | | (m/z) | |
| Number | Structure and Name | (M + H)⁺ | ¹H NMR and Rotation |
| I3 | 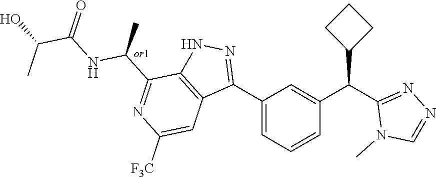 [SMILES: C[C@H](O)C(=O)N[C@@H](C)c1nc(C(F)(F)F)cc2c(-c3cccc([C@H](c4nncn4C)C4CCC4)c3)n[nH]c12] (2S)-N-{(1ξ)-1-[3-{3-[(R)-cyclobutyl(4-methyl-4H-1,2,4- triazol-3-yl)methyl]phenyl}-5-(trifluoromethyl)-1H- pyrazolo[3,4-c]pyridin-7-ylethyl}-2-hydroxypropanamide | 528.3 | ¹H NMR (400 MHz, DMSO- d₆) δ 14.39 (s, 1H), 8.39 − 8.32 (m, 2H), 8.29 (s, 1H), 7.91 (br d, J = 1.4 Hz, 2H), 7.51 (t, J = 7.9 Hz, 1H), 7.34 (br d, J = 7.6 Hz, 1H), 5.74 (d, J = 4.8 Hz, 1H), 5.55 (quin, J = 6.8 Hz, 1H), 4.37 (d, J = 10.6 Hz, 1H), 4.10 − 4.01 (m, 1H), 3.45 (s, 3H), 3.21 (br d, J = 10.0 Hz, 1H), 2.16 − 2.05 (m, 1H), 1.90 − 1.69 (m, 5H), 1.54 (d, J = 6.8 Hz, 3H), 1.18 (d, J = 6.8 Hz, 3H) |
| I4 | 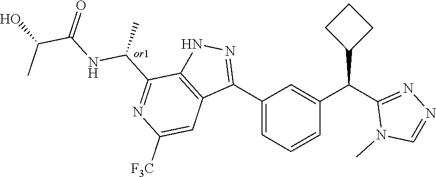 [SMILES: C[C@H](O)C(=O)N[C@H](C)c1nc(C(F)(F)F)cc2c(-c3cccc([C@H](c4nncn4C)C4CCC4)c3)n[nH]c12] (2S)-N-{(1ξ)-1-[3-{3-[(R)-cyclobutyl(4-methyl-4H-1,2,4- triazol-3-yl)methyl]phenyl}-5-(trifluoromethyl)-1H- pyrazolo[3,4-c]pyridin-7-yl]ethyl}-2-hydroxypropanamide | 528.3 | ¹H NMR (400 MHz, DMSO- d₆) δ 14.39 (br s, 1H), 8.45 (d, J = 7.3 Hz, 1H), 8.34 (s, 1H), 8.29 (s, 1H), 7.90 (br d, J = 2.0 Hz, 2H), 7.51 (t, J = 7.9 Hz, 1H), 7.34 (br d, J = 7.8 Hz, 1H), 5.71 − 5.52 (m, 2H), 4.37 (d, J = 10.5 Hz, 1H), 4.03 (br d, J = 6.6 Hz, 1H), 3.44 (s, 3H), 3.21 (br d, J = 10.6 Hz, 1H), 2.10 (br d, J = 6.6 Hz, 1H), 1.91 − 1.69 (m, 5H), 1.52 (d, J = 6.9 Hz, 3H), 1.30 − 1.22 (m, 3H) |
| I5 | 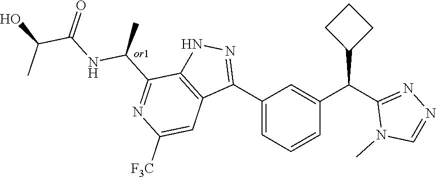 [SMILES: C[C@H](NC(=O)[C@@H](C)O)c1nc(C(F)(F)F)cc2c(-c3cccc([C@H](c4nncn4C)C4CCC4)c3)n[nH]c12] | 528.3 | ¹H NMR (400 MHz, DMSO-d₆) δ 14.39 (br s, 1H), 8.44 (d, J = 7.3 Hz, 1H), 8.34 (s, 1H), 8.29 (s, 1H), 7.97 − 7.81 (m, 2H), 7.51 (t, J = 7.9 Hz, 1H), 7.34 (d, J = 7.8 Hz, 1H), 5.67 − 5.52 (m, 2H), 4.37 (d, J = 10.5 Hz, 1H), 4.07 − 3.95 (m, 1H), 3.45 (s, 3H), 3.26 − 3.13 (m, 1H), 2.18 − 2.03 (m, 1H), 1.92 − 1.66 (m, 5H), 1.53 (d, J = 6.9 Hz, 3H), 1.26 (d, J = 6.8 Hz, 3H). |
| | (2R)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-2-hydroxypropanamide | | |
| I6 | 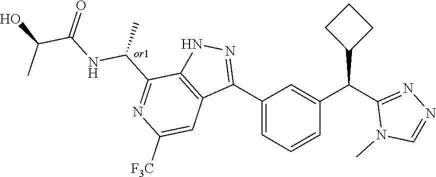 [SMILES: C[C@@H](O)C(=O)N[C@H](C)c1nc(C(F)(F)F)cc2c(-c3cccc([C@H](c4nncn4C)C4CCC4)c3)n[nH]c12] | 528.3 | ¹H NMR (400 MHz, DMSO-d₆) δ 14.39 (br s, 1H), 8.39 − 8.32 (m, 2H), 8.28 (s, 1H), 7.90 (br s, 2H), 7.51 (t, J = 7.9 Hz, 1H), 7.34 (d, J = 7.6 Hz, 1H), 5.73 (d, J = 4.9 Hz, 1H), 5.55 (t, J = 7.0 Hz, 1H), 4.37 (d, J = 10.5 Hz, 1H), 4.12 − 3.95 (m, 1H), 3.44 (s, 3H), 3.23 − 3.16 (m, 1H), 2.10 (br d, J = 7.1 Hz, 1H), 1.90 − 1.68 (m, 5H), 1.53 (d, J = 6.9 Hz, 3H), 1.18 (d, J = 6.9 Hz, 3H). |
| | (2R)-N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}-2-hydroxypropanamide | | |

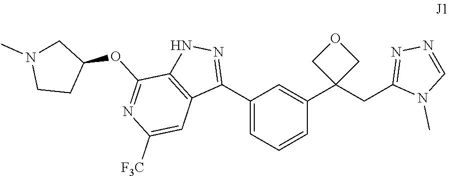 [SMILES: CN1CC[C@H](Oc2nc(C(F)(F)F)cc3c(-c4cccc(C5(Cc6nncn6C)COC5)c4)n[nH]c23)C1]

Example J1 was prepared according to General Method J.

To a mixture of 7-chloro-3-(3-{3-[(4-methyl-4H-1,2,4-triazol-3-yl)methyl]oxetan-3-yl}phenyl)-5-(trifluoromethyl)-1H-pyrazolo[3,4-c]pyridine (Intermediate 28) (33.0 mg, 0.074 mmol) in toluene (0.735 mL) was added (3S)-1-methylpyrrolidin-3-ol (22.1 mg, 0.221 mmol), and potassium methoxide (15.5 mg, 0.221 mmol) and the mixture was stirred at 80° C. for 18 h. Then, the reaction was concentrated, and the crude residue was purified by Prep HPLC (Phenomenex Gemini NX C18 150×21.2 mm, 5 um column Mobile phase A: Water+10 mM Ammonium Acetate Mobile phase B: Acetonitrile 40-100% B in 8.0 minutes, 40 mL/min) to provide 7-{[(3S)-1-methylpyrrolidin-3-yl]oxy}-3-(3-{3-[(4-methyl-4H-1,2,4-triazol-3-yl)methyl]oxetan-3-yl}phenyl)-5-(trifluoromethyl)-1H-pyrazolo[3,4-c]pyridine (Example J1) as a white solid (3.9 mg, 10%). LCMS (ESI+) 514.0 (M+H). ¹H NMR (600 MHz, DMSO-d₆) δ 8.13 (s, 1H), 7.82 (s, 1H), 7.80 (d, J=7.8 Hz, 1H), 7.53 (s, 1H), 7.40 (t, J=7.7 Hz, 1H), 6.96 (br d, J=7.8 Hz, 1H), 5.57 (td, J=5.3, 2.9 Hz, 1H), 4.95 (d, J=6.2 Hz, 2H), 4.89 (d, J=6.0 Hz, 2H), 3.52 (s, 3H), 2.92 (s, 3H), 2.80-2.76 (m, 3H), 2.35-2.32 (m, 4H), 1.96-1.90 (m, 2H). [α]_{D}²²=−2.8° (c 0.1, MeOH).

Examples J2-J8 reported in Table 7 were synthesized with non-critical changes or substitutions to the exemplified procedures for Example J1 that one skilled in the art would be able to realize.

TABLE 7

| | | LCMS | |
|---|---|---|---|
| Example | | (m/z) | |
| Number | Structure and Name | (M + H)⁺ | ¹H NMR and Rotation |
| J2 | 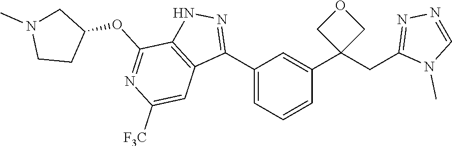 [SMILES: CN1CC[C@@H](Oc2nc(C(F)(F)F)cc3c(-c4cccc(C5(Cc6nncn6C)COC5)c4)n[nH]c23)C1] 7-{[(3S)-1-methylpyrrolidin-3- | 514.0 | ¹H NMR (600 MHz, DMSO-d₆) δ 8.16 8.10 (m, 1H), 7.78 (d, J = 7.8 Hz, 1H), 7.76 (s, 1H), 7.55 − 7.52 (m, 1H), 7.39 − 7.35 (m, 1H), 6.90 − 6.85 (m, 1H), 5.59 − 5.53 (m, 1H), 4.96 − 4.93 (m, 2H), 4.90 − 4.86 (m, 2H), 2.90 (s, 3H), 2.83 − 2.77 (m, 3H), 2.35 (br s, 4H), 2.25 − 2.24 (m, 3H), 1.96 − 1.94 (m, 2H). |
| | yl]oxy}-3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridine | | |
| J3 | 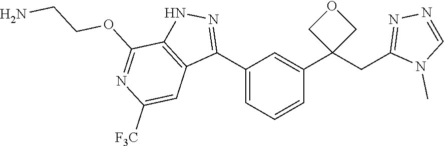 [SMILES: Cn1cnnc1CC1(c2cccc(-c3n[nH]c4c(OCCN)nc(C(F)(F)F)cc34)c2)COC1] | | ¹H NMR (600 MHz, DMSO-d₆) δ 8.16 (s, 1H), 7.91 (s, 1H), 7.85 (d, J = 7.9 Hz, 1H), 7.56 (t, J = 1.6 Hz, 1H), 7.46 (t, J = 7.7 Hz, 1H), 7.06 (d, J = 7.6 Hz, 1H), 4.98 (d, J = 6.2 Hz, 2H), 4.92 (d, J = 6.1 Hz, 2H), 4.54 (t, J = 5.6 Hz, 2H), 3.56 (s, 3H), 3.11 (br t, J = 5.6 Hz, 2H), 2.96 (s, 2H). |
| | 2-{[3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridin-7- | | |
| | yl]oxy}ethan-1-amine | | |
| J4 | 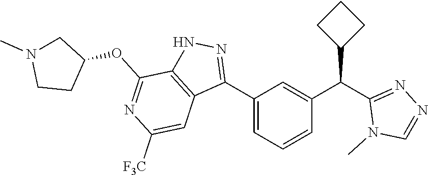 [SMILES: CN1CC[C@@H](Oc2nc(C(F)(F)F)cc3c(-c4cccc([C@H](c5nncn5C)C5CCC5)c4)n[nH]c23)C1] | 512.4 | ¹H NMR (400 MHz, CD₃OD) δ 8.37 (s, 1H), 7.87 − 7.76 (m, 3H), 7.51 (t, J = 7.8 Hz, 1H), 7.32 (d, J = 7.8 Hz, 1H), 5.79 − 5.69 (m, 1H), 4.31 (d, J = 11.0 Hz, 1H), 3.52 (s, 3H), 3.07 − 2.90 (m, 3H), 2.60 − 2.48 (m, 2H), 2.45 (s, 3H), 2.35 − 2.23 (m, 1H), 2.21 − 2.09 (m, 1H), 2.06 − 1.66 (m, 6H). |
| | 3-{3-[(R)-cyclobutyl(4-methyl- | | |
| | 4H-1,2,4-triazol-3- | | |
| | yl)methyl]phenyl}-7-{[(3R)-1- | | |
| | methylpyrrolidin-3-yl]oxy}-5- | | |
| | (trifluoromethyl)-1H- | | |
| | pyrazolo[3,4-c]pyridine | | |
| J5 | 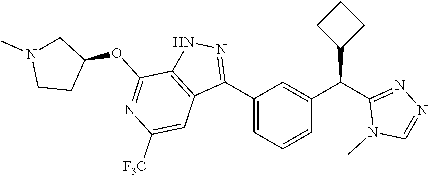 [SMILES: CN1CC[C@H](Oc2nc(C(F)(F)F)cc3c(-c4cccc([C@H](c5nncn5C)C5CCC5)c4)n[nH]c23)C1] | 512.4 | ¹H NMR (400 MHz, CD₃OD) δ 8.37 (s, 1H), 7.87 − 7.79 (m, 3H), 7.52 (t, J = 7.7 Hz, 1H), 7.33 (d, J = 7.8 Hz, 1H), 5.82 − 5.70 (m, 1H), 4.32 (d, J = 11.0 Hz, 1H), 3.53 (s, 3H), 3.08 − 2.90 (m, 3H), 2.61 − 2.49 (m, 2H), 2.46 (s, 3H), 2.34 − 2.24 (m, 1H), 2.22 − 2.10 (m, 1H), 2.04 − 1.72 (m, 6H). |
| | 3-{3-[(R)-cyclobutyl(4-methyl- | | |
| | 4H-1,2,4-triazol-3- | | |
| | yl)methyl]phenyl}-7-{[(3S)-1- | | |
| | methylpyrrolidin-3-yl]oxy}-5- | | |
| | (trifluoromethyl)-1H- | | |
| | pyrazolo[3,4-c]pyridine | | |
| J6 | 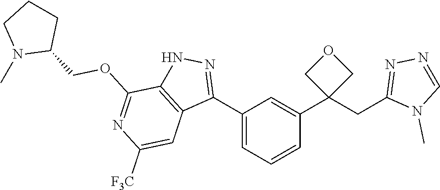 [SMILES: CN1CCC[C@@H]1COc1nc(C(F)(F)F)cc2c(-c3cccc(C4(Cc5nncn5C)COC4)c3)n[nH]c12] | 528.6 | |
| | 7-{[(2R)-1-methylpyrrolidin-2- | | |
| | yl]methoxy}-3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridine | | |
| J7 | 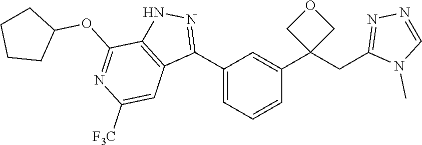 [SMILES: Cn1cnnc1CC1(c2cccc(-c3n[nH]c4c(OC5CCCC5)nc(C(F)(F)F)cc34)c2)COC1] | 499.5 | |
| | 7-(cyclopentyloxy)-3-(3-{3-[(4- | | |
| | methyl-4H-1,2,4-triazol-3- | | |
| | yl)methyl]oxetan-3-yl}phenyl)-5- | | |
| | (trifluoromethyl)-1H- | | |
| | pyrazolo[3,4-c]pyridine | | |
| J8 | 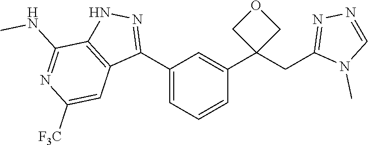 [SMILES: CNc1nc(C(F)(F)F)cc2c(-c3cccc(C4(Cc5nncn5C)COC4)c3)n[nH]c12] | 444.3 | ¹H NMR (400 MHz, CD₃OD) δ 8.15 (s, 1H), 7.80 (br d, J = 6.5 Hz, 1H), 7.49 (t, J = 7.7 Hz, 1H), 7.39 (br s, 2H), 6.95 (br d, J = 7.3 Hz, 1H), 5.14 − 5.08 (m, 4H), 3.67 (s, 2H), 3.14 (s, 3H), 2.87 (s, 3H). |
| | N-methyl-3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | amine | | |
| J9 | 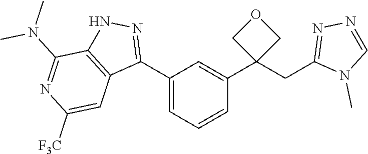 [SMILES: CN(C)c1nc(C(F)(F)F)cc2c(-c3cccc(C4(Cc5nncn5C)COC4)c3)n[nH]c12] | 458.3 | ¹H NMR (400 MHz, DMSO-d₆) δ 8.18 (s, 1H), 7.74 (br d, J = 7.5 Hz, 1H), 7.56 − 7.44 (m, 2H), 7.37 (br s, 1H), 7.08 (br d, J = 7.7 Hz, 1H), 5.01 − 4.96 (m, 2H), 4.96 − 4.91 (m, 2H), 3.56 (s, 2H), 3.43 (br s, 6H), 2.99 (s, 3H). |
| | N,N-dimethyl-3-(3-{3-[(4- | | |
| | methyl-4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7-amine | | |
| J10 | 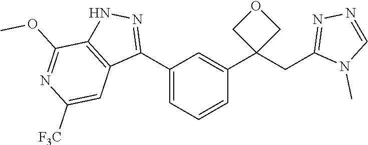 [SMILES: COc1nc(C(F)(F)F)cc2c(-c3cccc(C4(Cc5nncn5C)COC4)c3)n[nH]c12] | 445.2 | ¹H NMR (400 MHz, DMSO-d₆) δ 14.49 (br s, 1H), 8.18 (s, 1H), 7.93 (s, 1H), 7.86 (d, J = 7.8 Hz, 1H), 7.58 (s, 1H), 7.46 (t, J = 7.8 Hz, 1H), 7.06 (d, J = 7.9 Hz, 1H), 5.02 − 4.96 (m, 2H), 4.96 − 4.90 (m, 2H), 4.14 (s, 3H), 3.57 (s, 2H), 2.97 (s, 3H). |
| | 7-methoxy-3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridine | | |
| J11 | 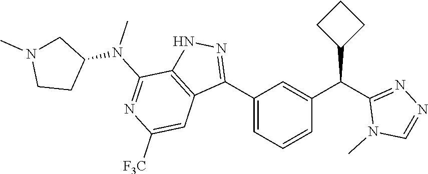 [SMILES: CN1CC[C@@H](N(C)c2nc(C(F)(F)F)cc3c(-c4cccc([C@H](c5nncn5C)C5CCC5)c4)n[nH]c23)C1] | 525.3 | ¹H NMR (400 MHz, CD₃OD) δ 8.37 (s, 1H), 7.72 (d, J = 7.9 Hz, 1H), 7.68 (s, 1H), 7.52 (t, J = 7.8 Hz, 1H), 7.39 (s, 1H), 7.32 (d, J = 7.8 Hz, 1H), 5.73 − 5.57 (m, 1H), 4.30 (d, J = 10.9 Hz, 1H), 3.57 − 3.50 (m, 3H), 3.40 (s, 3H), 3.03 − 2.95 (m, 1H), 2.90 − 2.77 (m, 2H), 2.74 − 2.63 (m, 1H), 2.44 (s, 3H), 2.37 − 2.20 (m, 2H), 2.13 − 1.72 (m, 7H). |
| | 3-{3-[(R)-cyclobutyl(4-methyl- | | |
| | 4H-1,2,4-triazol-3- | | |
| | yl)methyl]phenyl}-N-methyl-N- | | |
| | [(3R)-1-methylpyrrolidin-3-yl]-5- | | |
| | (trifluoromethyl)-1H- | | |
| | pyrazolo[3,4-c]pyridin-7-amine | | |
| J12 | 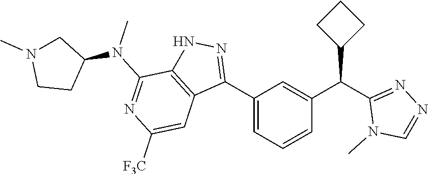 [SMILES: CN1CC[C@H](N(C)c2nc(C(F)(F)F)cc3c(-c4cccc([C@H](c5nncn5C)C5CCC5)c4)n[nH]c23)C1] 3-{3-[(R)-cyclobutyl(4-methyl- | 525.3 | ¹H NMR (400 MHz, CD₃OD) 8.37 (s, 1H), 7.73 (d, J = 7.8 Hz, 1H), 7.68 (s, 1H), 7.53 (t, J = 7.8 Hz, 1H), 7.41 (s, 1H), 7.34 (d, J = 7.8 Hz, 1H), 5.61 (br s, 1H), 4.61 (br s, 2H), 4.31 (d, J = 10.8 Hz, 1H), 3.53 (s, 3H), 3.44 (s, 3H), 3.12 − 3.03 (m, 1H), 3.02 − 2.87 (m, 2H), 2.85 − 2.73 (m, 1H), 2.52 (s, 3H), 2.43 − 2.23 (m, 2H), 2.18 − 2.06 (m, 1H), 2.00 − 1.86 (m, 4H), 1.85 − 1.74 (m, 1H). |
| | 4H-1,2,4-triazol-3- | | |
| | yl)methyl]phenyl}-N-methyl-N- | | |
| | [(3S)-1-methylpyrrolidin-3-yl]-5- | | |
| | (trifluoromethyl)-1H- | | |
| | pyrazolo[3,4-c]pyridin-7-amine | | |

### Example K1: (1R)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-amine

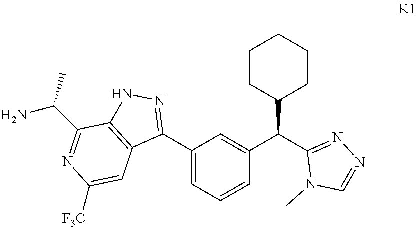 [SMILES: C[C@@H](N)c1nc(C(F)(F)F)cc2c(-c3cccc([C@H](c4nncn4C)C4CCCCC4)c3)n[nH]c12]

Example K1 was prepared according to General Method K.

To a mixture of tert-butyl {(1R)-1-[3-bromo-5-(trifluoromethyl)-1H-pyrazolo[3,4-c]pyridin-7-yl]ethyl}carbamate (Intermediate 33) (151 mg, 0.368 mmol), 3-{(R)-cyclohexyl[3-(4,4,5,5-tetramethyl-1,3,2-dioxaborolan-2-yl)phenyl]methyl}-4-methyl-4H-1,2,4-triazole (Intermediate 17′) (135 mg, 0.368 mmol), and Pd(dppf)Cl₂·CH₂Cl₂ (26.9 mg, 0.0368 equiv) in dioxane (3.68 mL) was added potassium carbonate (2M aqueous, 0.368 mL, 0.736 mmol). The mixture was degassed (3×) and stirred at 100° C. for 18 h. Then, the reaction was cooled, quenched with brine (10 mL), and extracted with DCM (2×10 mL). The organic layers were dried over Na₂SO₄, filtered, and concentrated under reduced pressure. The crude residue was purified by flash column chromatography (ISCO, 12 g SiO₂, 1:1 DCM:EtOAc/MeOH 0-10%) to give tert-butyl {(1R)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethyl}carbamate (57 mg) which was dissolved in DCM (2 mL) and HCl (1.84 mL, 4.0 M, 7.36 mmol) was added and the mixture was stirred and rt for 3 h. Then, the reaction was concentrated and diluted with DCM (3 mL). Saturated aqueous NaHCO₃ (3 mL) was added and the layers were separated. The organic layer was dried over Na₂SO₄, filtered, and concentrated under reduced pressure. The crude residue was purified by preparative HPLC (Prep HPLC (Phenomenex Gemini NX C18 150×21.2 mm, 5 um column Mobile phase A: Water+10 mM Ammonium Acetate Mobile phase B: Acetonitrile 35-100% B in 8.0 minutes, 40 mL/min) to provide (1R)-1-[3-{3-[(R)-cyclohexyl(4-methyl-4H-1,2,4-triazol-3-yl)methyl]phenyl}-5-(trifluoromethyl)-1H-pyrazolo[3,4-c]pyridin-7-yl]ethan-1-amine (Example K) as a white solid (19.7 mg, 11%). LMS (ESI+) 484.2 (M+H). H NMR (600 MHz, DMSO-d₆) 8.32 (s, 1H), 8.20 (s, 1H), 8.00 (s, 1H), 7.86 (d, J=7.6 Hz, 1H), 7.56-7.46 (m, 1H), 7.42 (br. d, J=8.0 Hz, 1H), 4.59 (q, J=6.5 Hz, 1H), 4.07 (d, J=9.8 Hz, 1H), 3.53 (m, 3H), 2.36-2.22 (m, 1H), 1.73-1.54 (m, 5H), 1.47 (d, J=6.9 Hz, 3H), 1.29-1.19 (m, 1H), 1.14 (q, J=10.1 Hz, 2H), 1.09-1.02 (m, 1H), 1.01-−0.90 (n, 1H).

Examples K2-K17 reported in Table 8 were synthesized with non-critical changes or substitutions to the exemplified procedures for Example K1 that one skilled in the art would be able to realize. In some cases, the protecting group (Boc) was not present (K9-11) or was not removed (K8).

TABLE 8

| | | LCMS | |
|---|---|---|---|
| Example | | (m/z) | |
| Number | Structure and Name | (M + H)⁺ | ¹H NMR and Rotation |
| K2 | 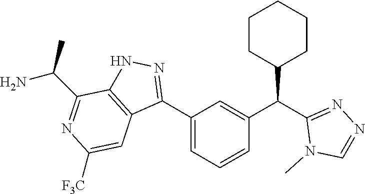 [SMILES: C[C@H](N)c1nc(C(F)(F)F)cc2c(-c3cccc([C@H](c4nncn4C)C4CCCCC4)c3)n[nH]c12] | 484.3 | ¹H NMR (600 MHz, DMSO-d₆) δ 8.32 (s, 1H), 8.20 (s, 1H), 8.00 (s, 1H), 7.86 (d, J = 7.6 Hz, 1H), 7.56 − 7.46 (m, 1H), 7.42 (br. d, J = 8.0 Hz, 1H), 4.59 (q, J = 6.5 Hz, 1H), 4.07 (d, J = 9.8 Hz, 1H), 3.53 (s, 3H), 2.36 − 2.22 (m, 1H), 1.73 − 1.54 (m, 5H), 1.47 (d, J = 6.9 Hz, 3H), 1.29 − 1.19 (m, 1H), 1.14 (q, J = 10.1 Hz, 2H), 1.09 − 1.02 (m, 1H), 1.01 − 0.90 (m, 1H). |
| | (1S)-1-[3-{3-[(R)-cyclohexyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]ethan-1-amine | | |
| K3 | 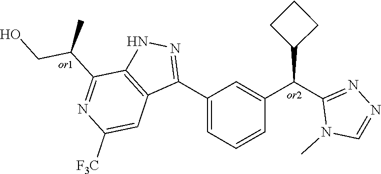 [SMILES: C[C@H](CO)c1nc(C(F)(F)F)cc2c(-c3cccc([C@H](c4nncn4C)C4CCC4)c3)n[nH]c12] | 471.1 | ¹H NMR (400 MHz, DMSO-d₆) δ 14.29 (s, 1H), 8.19 (s, 1H), 7.88 (br d, J = 5.6 Hz, 2H), 7.50 (br t, J = 7.8 Hz, 1H), 7.32 (br d, J = 7.4 Hz, 1H), 4.79 (br s, 1H), 4.40 (br d, J = 9.8 Hz, 1H), 3.89 − 3.79 (m, 1H), 3.77 − 3.66 (m, 2H), 3.47 − 3.43 (m, 3H), 3.22 − 3.15 (m, 1H), 2.10 (br s, 1H), 1.88 − 1.65 (m, 6H), 1.33 (br d, J = 6.5 Hz, 3H). |
| | (2ξ)-2-[3-{3-[(ξ)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]propan-1-ol | | |
| K4 | 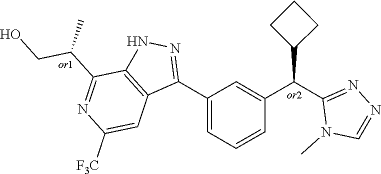 [SMILES: C[C@@H](CO)c1nc(C(F)(F)F)cc2c(-c3cccc([C@H](c4nncn4C)C4CCC4)c3)n[nH]c12] | 471.1 | ¹H NMR (400 MHz, DMSO-d₆) δ 8.38 − 8.31 (m, 1H), 8.20 (br s, 1H), 7.89 (br s, 2H), 7.50 (br s, 1H), 7.33 (br d, J = 6.0 Hz, 1H), 4.37 (br d, J = 9.5 Hz, 1H), 3.83 (br s, 1H), 3.72 (br s, 2H), 3.44 (br s, 3H), 3.21 (br dd, J = 1.9, 3.1 Hz, 1H), 2.10 (br d, J = 2.3 Hz, 1H), 1.80 (br s, 5H), 1.33 (br s, 3H). |
| | (2ξ)-2-[3-{3-[(ξ)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]propan-1-ol | | |
| K5 | 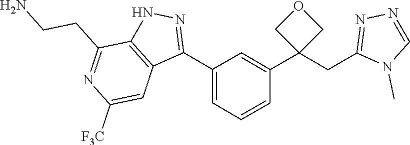 [SMILES: Cn1cnnc1CC1(c2cccc(-c3n[nH]c4c(CCN)nc(C(F)(F)F)cc34)c2)COC1] | 458.1 | ¹H NMR (600 MHz, DMSO-d₆) δ 8.12 − 8.09 (m, 1H), 8.08 (s, 1H), 7.82 (d, J = 7.7 Hz, 1H), 7.53 (s, 1H), 7.38 (t, J = 7.6 Hz, 1H), 6.94 (br. d, J = 7.7 Hz, 1H), 4.93 (d, J = 6.0 Hz, 2H), 4.87 (d, J = 6.0 Hz, 2H), 3.50 (s, 2H), 3.34 − 3.29 (m, 2H), 3.12 (br. d, J = 6.2 Hz, 2H), 2.88 (s, 3H). |
| | 2-[3-(3-{3-[(4-methyl-4H-1,2,4- | | |
| | triazol-3-yl)methyl]oxetan-3- | | |
| | yl}phenyl)-5-(trifluoromethyl)- | | |
| | 1H-pyrazolo[3,4-c]pyridin-7- | | |
| | yl]ethan-1-amine | | |
| K6 | 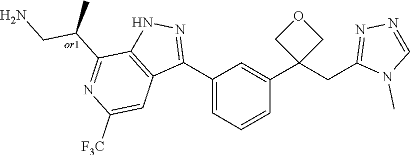 [SMILES: C[C@H](CN)c1nc(C(F)(F)F)cc2c(-c3cccc(C4(Cc5nncn5C)COC4)c3)n[nH]c12] | 472.0 | ¹H NMR (600 MHz, DMSO-d₆) δ 8.17 (s, 1H), 8.14 (s, 1H), 7.88 (d, J = 7.8 Hz, 1H), 7.58 (s, 1H), 7.46 (t, J = 7.7 Hz, 1H), 7.05 (br. d, J = 7.7 Hz, 1H), 5.07 − 4.97 (m, 2H), 4.94 (d, J = 6.1 Hz, 2H), 3.57 (s, 4H), 3.52 − 3.36 (m, 1H), 3.20 − 3.06 (m, 1H), 2.96 (s, 3H), 1.36 (d, J = 6.8 Hz, 2H). [α]_{D}²² +29.7° (c 0.1, MeOH). |
| | (2ξ)-2-[3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]propan-1-amine | | |
| K7 | 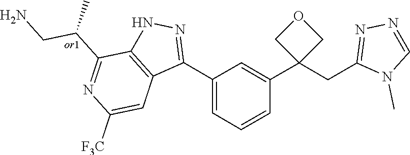 [SMILES: C[C@@H](CN)c1nc(C(F)(F)F)cc2c(-c3cccc(C4(Cc5nncn5C)COC4)c3)n[nH]c12] (2ξ)-2-[3-(3-{3-[(4-methyl-4H- | 472.0 | ¹H NMR (600 MHz, DMSO-d₆) δ 8.17 (s, 1H), 8.13 (s, 1H), 7.88 (d, J = 7.8 Hz, 1H), 7.58 (s, 1H), 7.46 (t, J = 7.8 Hz, 1H), 7.04 (br. d, J = 7.8 Hz, 1H), 5.06 − 4.97 (m, 2H), 4.94 (d, J = 6.1 Hz, 2H), 3.63 − 3.57 (m, 4H), 3.52 − 3.47 (m, 1H), 3.18 − 3.07 (m, 1H), 3.01 − 2.93 (m, 3H), 1.36 (d, J = 6.8 Hz, 2H). [α]_{D}²² −29.0° (c 0.1, MeOH). |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]propan-1-amine | | |
| K8 | 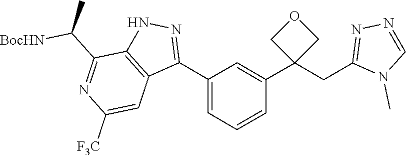 [SMILES: C[C@H](NC(=O)OC(C)(C)C)c1nc(C(F)(F)F)cc2c(-c3cccc(C4(Cc5nncn5C)COC4)c3)n[nH]c12] | 558.2 | ¹H NMR (400 MHz, CDCl₃) δ 8.08 (s, 1H), 7.90 (s, 1H), 7.79 (d, J = 7.8 Hz, 1H), 7.50 (t, J = 1.4 Hz, 1H), 7.43 (t, J = 7.8 Hz, 1H), 6.84 (br d, J = 7.8 Hz, 1H), 5.20 − 5.09 (m, 4H), 3.62 (s, 2H), 2.82 (s, 3H), 2.23 (s, 3H), 1.73 (d, J = 6.9 Hz, 3H), 1.24 (s, 9H). |
| | tert-butyl {(1S)-1-[3-(3-{3-[(4- | | |
| | methyl-4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7- | | |
| | ylethyl}carbamate | | |
| K9 | 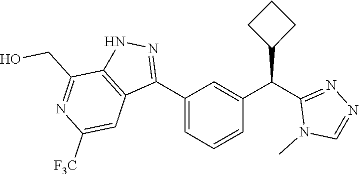 [SMILES: Cn1cnnc1[C@@H](c1cccc(-c2n[nH]c3c(CO)nc(C(F)(F)F)cc23)c1)C1CCC1] | 443.0 | |
| | [3-{3-[(R)-cyclobutyl(4-methyl- | | |
| | 4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]methanol | | |
| K10 | 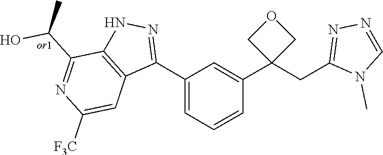 [SMILES: C[C@H](O)c1nc(C(F)(F)F)cc2c(-c3cccc(C4(Cc5nncn5C)COC4)c3)n[nH]c12] | 459.1 | ¹H NMR (600 MHz, DMSO-d₆) δ 8.20 (s, 1H), 8.17 (s, 1H), 7.88 (d, J = 7.9 Hz, 1H), 7.58 (s, 1H), 7.47 (t, J = 7.7 Hz, 1H), 7.07 (d, J = 8.3 Hz, 1H), 5.97 (br. s, 1H), 5.20 (q, J = 6.3 Hz, 1H), 5.00 (d, J = 6.1 Hz, 2H), 4.94 (d, J = 6.1 Hz, 2H), 3.57 (s, 2H), 2.97 (s, 3H), 1.56 (d, J = 6.6 Hz, 3H). [α]_{D}²² −3.4° (c 0.1, MeOH). |
| | (1ξ)-1-[3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]ethan-1-ol | | |
| K11 | 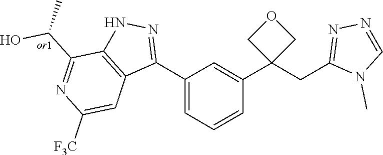 [SMILES: C[C@@H](O)c1nc(C(F)(F)F)cc2c(-c3cccc(C4(Cc5nncn5C)COC4)c3)n[nH]c12] (1ξ)-1-[3-(3-{3-[(4-methyl-4H- | 459.1 | ¹H NMR (600 MHz, DMSO-d₆) δ 8.21 (s, 1H), 8.17 (s, 1H), 7.88 (d, J = 7.7 Hz, 1H), 7.58 (s, 1H), 7.48 (t, J = 7.7 Hz, 1H), 7.08 (d, J = 7.7 Hz, 1H), 5.96 (br. d, J = 2.4 Hz, 1H), 5.25 − 5.15 (m, 1H), 5.00 (d, J = 6.1 Hz, 2H), 4.94 (d, J = 6.1 Hz, 2H), 3.57 (s, 2H), 2.97 (s, 3H), 1.56 (d, J = 6.6 Hz, 3H). [α]_{D}²² +1.6° (c 0.1, MeOH). |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]ethan-1-ol | | |
| K12 | 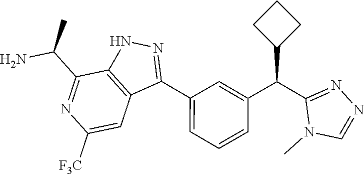 [SMILES: C[C@H](N)c1nc(C(F)(F)F)cc2c(-c3cccc([C@H](c4nncn4C)C4CCC4)c3)n[nH]c12] | 456.3 | ¹H NMR (400 MHz, DMSO-d₆) δ 14.88 (br s, 1H), 9.05 (br s, 1H), 8.69 (br s, 3H), 8.44 (s, 1H), 8.03 − 7.91 (m, 2H), 7.56 (t, J = 7.6 Hz, 1H), 7.38 (br d, J = 7.8 Hz, 1H), 5.23 (br s, 1H), 4.60 (br d, J = 10.6 Hz, 1H), 3.58 (s, 3H), 3.22 (br d, J = 9.2 Hz, 1H), 2.13 (br s, 1H), 1.95 − 1.72 (m, 5H), 1.64 (br d, J = 6.7 Hz, 3H). |
| | (1S)-1-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]ethan-1-amine | | |
| K13 | 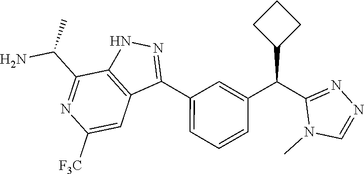 [SMILES: C[C@@H](N)c1nc(C(F)(F)F)cc2c(-c3cccc([C@H](c4nncn4C)C4CCC4)c3)n[nH]c12] | 456.3 | ¹H NMR (400 MHz, DMSO-d₆) δ 14.89 (br s, 1H), 9.07 (br s, 1H), 8.70 (br s, 3H), 8.44 (s, 1H), 8.03 − 7.91 (m, 2H), 7.56 (t, J = 7.6 Hz, 1H), 7.37 (br d, J = 7.6 Hz, 1H), 5.24 (br s, 1H), 4.61 (br d, J = 10.5 Hz, 1H), 3.59 (s, 3H), 3.27 − 3.16 (m, 1H), 2.13 (br s, 1H), 1.87 − 1.81 (m, 5H), 1.65 (br d, J = 6.7 Hz, 3H). |
| | (1R)-1-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]ethan-1-amine | | |
| K14 | 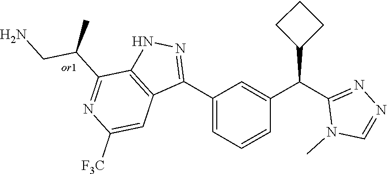 [SMILES: C[C@H](CN)c1nc(C(F)(F)F)cc2c(-c3cccc([C@H](c4nncn4C)C4CCC4)c3)n[nH]c12] | 470.4 | ¹H NMR (400 MHz, DMSO-d₆) δ 14.73 (br s, 1H), 9.22 (br s, 1H), 8.31 (s, 1H), 8.11 (br s, 3H), 7.99 − 7.90 (m, 2H), 7.55 (t, J = 7.7 Hz, 1H), 7.36 (d, J = 7.5 Hz, 1H), 4.66 (d, J = 10.8 Hz, 1H), 4.04 − 3.90 (m, 1H), 3.61 (s, 3H), 3.49 (br d, J = 5.3 Hz, 1H), 3.23 (br d, J = 6.8 Hz, 2H), 2.13 (br d, J = 6.3 Hz, 1H), 1.90 − 1.73 (m, 5H), 1.42 (d, J = 6.8 Hz, 3H). |
| | (2ξ)-2-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]propan-1-amine | | |
| K15 | 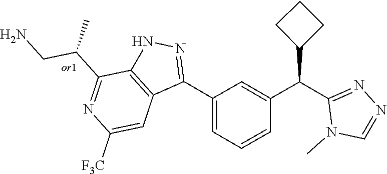 [SMILES: C[C@@H](CN)c1nc(C(F)(F)F)cc2c(-c3cccc([C@H](c4nncn4C)C4CCC4)c3)n[nH]c12] | 470.4 | ¹H NMR (400 MHz, DMSO-d₆) δ 14.77 (br s, 1H), 9.30 (s, 1H), 8.33 (s, 1H), 8.16 (br s, 3H), 8.01 − 7.92 (m, 2H), 7.56 (t, J = 7.7 Hz, 1H), 7.37 (d, J = 7.8 Hz, 1H), 4.68 (d, J = 10.5 Hz, 1H), 4.07 − 3.94 (m, 1H), 3.62 (s, 3H), 3.56 − 3.42 (m, 1H), 3.30 − 3.14 (m, 2H), 2.14 (br d, J = 3.8 Hz, 1H), 1.95 − 1.74 (m, 5H), 1.42 (d, J = 6.8 Hz, 3H). |
| | (2ξ)-2-[3-{3-[(R)-cyclobutyl(4- | | |
| | methyl-4H-1,2,4-triazol-3- | | |
| | yl)methyl]phenyl}-5- | | |
| | (trifluoromethyl)-1H- | | |
| | pyrazolo[3,4-c]pyridin-7- | | |
| | yl]propan-1-amine | | |
| K16 | 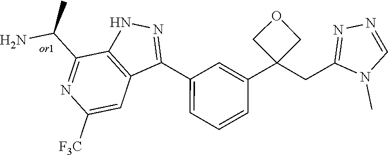 [SMILES: C[C@H](N)c1nc(C(F)(F)F)cc2c(-c3cccc(C4(Cc5nncn5C)COC4)c3)n[nH]c12] | 480.2 | ¹H NMR (400 MHz, DMSO-d₆) δ 8.19 (s, 2H), 7.89 (br d, J = 7.5 Hz, 1H), 7.59 (s, 1H), 7.48 (t, J = 7.1 Hz, 1H), 7.07 (br d, J = 7.5 Hz, 1H), 5.04 − 4.97 (m, 2H), 4.95 (br d, J = 6.0 Hz, 2H), 4.60 (q, J = 6.5 Hz, 1H), 3.58 (br s, 2H), 2.97 (s, 3H), 1.48 (d, J = 6.5 Hz, 3H). |
| | (1ξ)-1-[3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]ethan-1-amine | | |
| K17 | 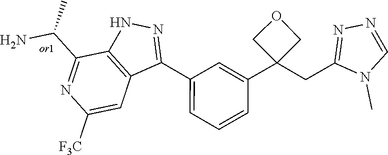 [SMILES: C[C@@H](N)c1nc(C(F)(F)F)cc2c(-c3cccc(C4(Cc5nncn5C)COC4)c3)n[nH]c12] | 480.2 | ¹H NMR (400 MHz, DMSO-d₆) δ 8.21 (s, 1H), 8.19 (s, 1H), 7.90 (d, J = 7.8 Hz, 1H), 7.59 (s, 1H), 7.48 (t, J = 7.8 Hz, 1H), 7.08 (d, J = 8.0 Hz, 1H), 5.03 − 4.97 (m, 2H), 4.97 − 4.92 (m, 2H), 4.63 (q, J = 6.7 Hz, 1H), 3.57 (s, 2H), 2.97 (s, 3H), 1.49 (d, J = 6.8 Hz, 3H). |
| | (1ξ)-1-[3-(3-{3-[(4-methyl-4H- | | |
| | 1,2,4-triazol-3-yl)methyl]oxetan- | | |
| | 3-yl}phenyl)-5-(trifluoromethyl)- | | |
| | 1H-pyrazolo[3,4-c]pyridin-7- | | |
| | yl]ethan-1-amine | | |

### Example L1: 3-{3-[(R)-cyclobutyl(4-methyl-4H-1,2,4-triazol-3-yl)methyl]phenyl}-7-[(2ξ)-piperidin-2-yl]-5-(trifluoromethyl)-1H-pyrazolo[3,4-c]pyridine

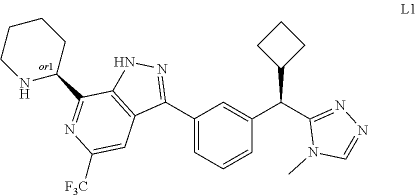 [SMILES: Cn1cnnc1[C@@H](c1cccc(-c2n[nH]c3c([C@@H]4CCCCN4)nc(C(F)(F)F)cc23)c1)C1CCC1]

Example L1 was prepared according to General Method L.

### Step 1: tert-butyl (2ξ)-2-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]piperidine-1-carboxylate

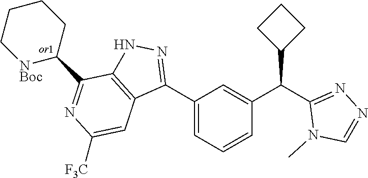 [SMILES: Cn1cnnc1[C@@H](c1cccc(-c2n[nH]c3c([C@@H]4CCCCN4C(=O)OC(C)(C)C)nc(C(F)(F)F)cc23)c1)C1CCC1]

To a vial was added (2S)-1-(tert-butoxycarbonyl)piperidine-2-carboxylic acid (64.6 mg, 0.282 mmol), 7-chloro-3-{3-[(R)-cyclobutyl(4-methyl-4H-1,2,4-triazol-3-yl)methyl]phenyl}-5-(trifluoromethyl)-1H-pyrazolo[3,4-c]pyridine (Intermediate 30) (84 mg, 0.19 mmol), [4,4′-Bis(1,1-dimethylethyl)-2,2′-bipyridine-N₁,N₁′]bis[3,5-difluoro-2-[5-(trifluoromethyl)-2-pyridinyl-N]phenyl-C]Iridium(III) hexafluorophosphate (2.1 mg, 0.00188 mmol), [4,4′-Bis(1,1-dimethylethyl)-2,2′-bipyridine]nickel (II) dichloride (3.8 mg, 0.0094 mmol), Cs₂CO₃ (91.9 mg, 0.282 mmol) and DMF (3.76 mL). The reaction was degassed and irradiated with Pennoc photoreactor at 100% power at room temperature for 18 h. The reaction was quenched with water (15 mL) and extracted with EtOAc (2×15 mL). The combined organic layers were dried over Na₂SO₄, filtered, and concentrated under reduced pressure. The crude residue was purified via flash column chromatography (Isco, 0-100% heptane/EtOAc) followed by preparative SFC (Column: ChiralPak IC, 250×21 mm, 5 um; Flow rate: 95 mL/min; 17% MeOH (with 10 mM NH₃) in CO₂). Peak 1: tert-butyl (2ξ)-2-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]piperidine-1-carboxylate was isolated as a white solid (11 mg, 10%). LCMS (ESI+) 596.2 (M+H). [α]_{D}²² −17.6° (c 0.1, MeOH).

### Step 2: 3-{3-[(R)-cyclobutyl(4-methyl-4H-1,2,4-triazol-3-yl)methyl]phenyl}-7-[(2ξ)-piperidin-2-yl]-5-(trifluoromethyl)-1H-pyrazolo[3,4-c]pyridine (Example L1)

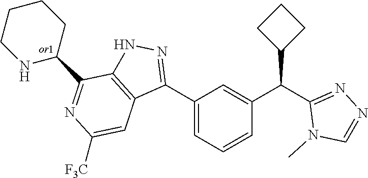 [SMILES: Cn1cnnc1[C@@H](c1cccc(-c2n[nH]c3c([C@@H]4CCCCN4)nc(C(F)(F)F)cc23)c1)C1CCC1]

To a solution of tert-butyl (2ξ)-2-[3-{3-[(R)-cyclobutyl(4-methyl-4H-1,2,4-triazol-3-yl)methyl]phenyl}-5-(trifluoromethyl)-1H-pyrazolo[3,4-c]pyridin-7-yl]piperidine-1-carboxylate (11 mg, 0.018 mmol) in DCM (1 mL) was added HCl (0.138 mL, 4 M, 0.554 mmol) and the reaction was stirred at room temperature for 2 h. Then, the reaction was concentrated under reduced pressure. Toluene was added and the reaction was concentrated again (3×) before being lyophilized from water and MeOH to obtain 3-{3-[(R)-cyclobutyl(4-methyl-4H-1,2,4-triazol-3-yl)methyl]phenyl}-7-[(2ξ)-piperidin-2-yl]-5-(trifluoromethyl)-1H-pyrazolo[3,4-c]pyridine (Example L1) as the HCl salt (9.2 mg, 94%) and a white solid. LCMS (ESI+) 496.1 (M+H). ¹H NMR (600 MHz, DMSO-d₆) δ 9.76-9.42 (m, 1H), 9.03-8.77 (m, 1H), 8.63 (s, 1H), 8.36 (s, 1H), 8.06-7.88 (m, 2H), 7.55 (t, J=7.6 Hz, 1H), 7.41 (d, J=7.6 Hz, 1H), 5.22-5.10 (m, 1H), 4.47 (d, J=10.3 Hz, 1H), 3.56 (s, 2H), 3.53-3.50 (m, 1H), 3.21-3.17 (m, 1H), 2.43-2.30 (m, 1H), 2.22-2.11 (m, 1H), 1.99-1.76 (m, 10H).

Examples L2-L4 reported in Table 9 were synthesized with non-critical changes or substitutions to the exemplified procedures for Example L1 that one skilled in the art would be able to realize.

TABLE 9

| | | LCMS | |
|---|---|---|---|
| Example | | (m/z) | |
| Number | Structure and Name | (M + H)⁺ | ¹H NMR and Rotation |
| L2 | 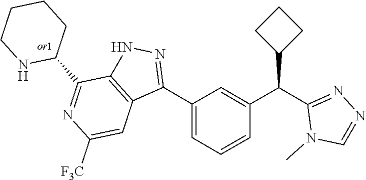 [SMILES: Cn1cnnc1[C@@H](c1cccc(-c2n[nH]c3c([C@H]4CCCCN4)nc(C(F)(F)F)cc23)c1)C1CCC1] | 496.1 | ¹H NMR (600 MHz, DMSO-d₆) δ 9.72 (br. D, J = 9.0 Hz, 1H), 9.26 − 8.99 (m, 2H), 8.53 (s, 1H), 8.19 − 8.01 (m, 2H), 7.64 (br. T, J = 7.5 Hz, 1H), 7.47 (br. D, J = 7.5 Hz, 1H), 5.23 (br. T, J = 10.5 Hz, 1H), 4.67 (br. D, J = 10.1 Hz, 1H), 3.67 (br. S, 3H), 3.36 − 3.30 (m, 1H), 3.21 (br. D, J = 2.2 Hz, 1H), 2.50 − 2.37 (m, 1H), 2.22 (br. S, 1H), 1.97 − 1.69 (m, 9H). |
| | 3-{3-[(R)-cyclobutyl(4-methyl- | | |
| | 4H-1,2,4-triazol-3- | | |
| | yl)methyl]phenyl}-7-[(2ξ)- | | |
| | piperidin-2-yl]-5- | | |
| | (trifluoromethyl)-1H- | | |
| | pyrazolo[3,4-c]pyridine | | |
| L3 | 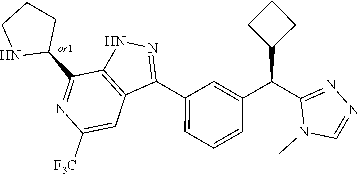 [SMILES: Cn1cnnc1[C@@H](c1cccc(-c2n[nH]c3c([C@@H]4CCCN4)nc(C(F)(F)F)cc23)c1)C1CCC1] | 482.1 | |
| | 3-{3-[(R)-cyclobutyl(4-methyl- | | |
| | 4H-1,2,4-triazol-3- | | |
| | yl)methyl]phenyl}-7-[(2ξ)- | | |
| | pyrrolidin-2-yl]-5- | | |
| | (trifluoromethyl)-1H- | | |
| | pyrazolo[3,4-c]pyridine | | |
| L4 | 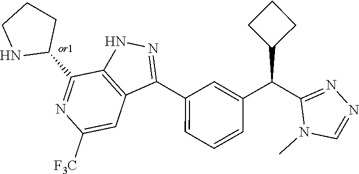 [SMILES: Cn1cnnc1[C@@H](c1cccc(-c2n[nH]c3c([C@H]4CCCN4)nc(C(F)(F)F)cc23)c1)C1CCC1] | 482.1 | |
| | 3-{3-[(R)-cyclobutyl(4-methyl- | | |
| | 4H-1,2,4-triazol-3- | | |
| | yl)methyl]phenyl}-7-[(2ξ)- | | |
| | pyrrolidin-2-yl]-5- | | |
| | (trifluoromethyl)-1H- | | |
| | pyrazolo[3,4-c]pyridine | | |

### Example M1: N-{(1ξ)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}acetamide

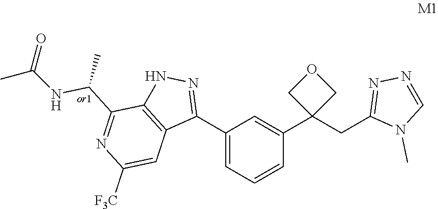 [SMILES: CC(=O)N[C@H](C)c1nc(C(F)(F)F)cc2c(-c3cccc(C4(Cc5nncn5C)COC4)c3)n[nH]c12]

Example M1 was prepared according to General Method M.

Stock solutions of acetyl chloride (16 uL) in DMF (1 mL) and i-Pr₂NEt (76 uL) in DMF (1 mL) were prepared. A 0.1 mL aliquot of each solution were added to (1ξ)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethan-1-amine (Example K17) (10 mg, 0.022 mmol). The mixture was stirred at rt for 18 h. An additional 0.1 mL aliquot of a freshly prepared stock solution of acetyl chloride (16 uL) in DMF (1 mL) was added and the reaction was stirred at rt for 2 h. The mixture was diluted with MeOH and purified via reverse-phase flash column chromatography to give N-{(1ξ)-1-[3-(3-{3-[(4-methyl-4H-1,2,4-triazol-3-yl)methyl]oxetan-3-yl}phenyl)-5-(trifluoromethyl)-1H-pyrazolo[3,4-c]pyridin-7-yl]ethyl}acetamide (Example M1) (6 mg, 50%) as a white solid. LCMS (ESI+) 500.2 (M+H)₂. ¹H NMR (600 MHz, DMSO-d₆) δ 14.24 (br. s, 1H), 8.56 (d, J=7.0 Hz, 1H), 8.19 (s, 1H), 8.17 (s, 1H), 7.89 (d, J=7.7 Hz, 1H), 7.58 (s, 1H), 7.47 (t, J=7.7 Hz, 1H), 7.07 (d, J=7.7 Hz, 1H), 5.51 (p, J=7.0 Hz, 1H), 5.00 (d, J=6.0 Hz, 2H), 4.94 (dd, J=6.1, 2.1 Hz, 2H), 3.57 (s, 2H), 2.95 (s, 3H), 1.88 (s, 3H), 1.50 (d, J=7.0 Hz, 3H).

Examples M2-M6 reported in Table 10 were synthesized with non-critical changes or substitutions to the exemplified procedures for Example M1 that one skilled in the art would be able to realize.

TABLE 10

| | | LCMS | |
|---|---|---|---|
| Example | | (m/z) | |
| Number | Structure and Name | (M + H)⁺ | ¹H NMR and Rotation |
| M2 | 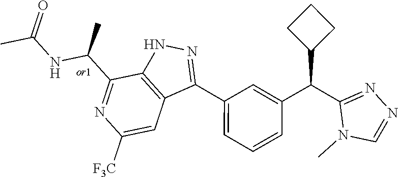 [SMILES: CC(=O)N[C@@H](C)c1nc(C(F)(F)F)cc2c(-c3cccc([C@H](c4nncn4C)C4CCC4)c3)n[nH]c12] | 498.2 | ¹H NMR (400 MHz, DMSO) δ 14.21 (br. s, 1H), 8.52 (d, J = 6.9 Hz, 1H), 8.33 (s, 1H), 8.24 (s, 1H), 7.91 − 7.86 (m, 2H), 7.53 − 7.48 (m, 1H), 7.33 (dt, J = 7.7, 1.5 Hz, 1H), 5.52 (p, J = 7.0 Hz, 1H), 4.37 (d, J = 10.5 Hz, 1H), 3.45 (s, 3H), 3.27 − 3.16 (m, 1H), 2.15 − 2.06 (m, 1H), 1.89 − 1.70 (m, 8H), 1.50 (d, J = 7.0 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]ethyl}acetamide | | |
| M3 | 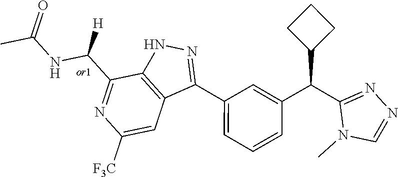 | 498.2 | ¹H NMR (400 MHz, DMSO-d₆) δ 14.22 (br. s, 1H), 8.52 (d, J = 6.9 Hz, 1H), 8.33 (s, 1H), 8.24 (s, 1H), 7.91 − 7.87 (m, 2H), 7.51 (t, J = 7.9 Hz, 1H), 7.34 (br. dt, J = 7.8, 1.5 Hz, 1H), 5.52 (p, J = 6.9 Hz, 1H), 4.37 (d, J = 10.5 Hz, 1H), 3.45 (s, 3H), 3.26 − 3.15 (m, 1H), 2.15 − 2.05 (m, 1H), 1.88 (s, 3H), 1.86 − 1.70 (m, 5H), 1.50 (d, J = 7.0 Hz, 3H). |
| | N-{(1ξ)-1-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | ylethyl}acetamide | | |
| M4 | 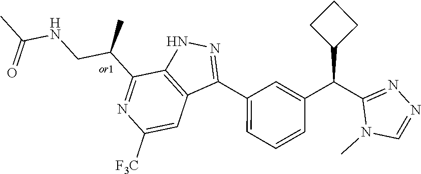 [SMILES: CC(=O)NC[C@@H](C)c1nc(C(F)(F)F)cc2c(-c3cccc([C@H](c4nncn4C)C4CCC4)c3)n[nH]c12] | 512.2 | ¹H NMR (400 MHz, DMSO-d₆) δ 14.30 (br d, J = 5.1 Hz, 1H), 8.34 (s, 1H), 8.20 (s, 1H), 7.94 − 7.85 (m, 3H), 7.55 − 7.47 (m, 1H), 7.34 (d, J = 7.3 Hz, 1H), 4.38 (d, J = 10.4 Hz, 1H), 3.76 − 3.67 (m, 1H), 3.56 − 3.49 (m, 2H), 3.46 (s, 3H), 2.16 − 2.05 (m, 1H), 1.93 − 1.77 (m, 5H), 1.75 (s, 3H), 1.35 (d, J = 6.9 Hz, 3H). |
| | N-{(2ξ)-2-[3-{3-[(R)- | | |
| | cyclobutyl(4-methyl-4H-1,2,4- | | |
| | triazol-3-yl)methyl]phenyl}-5- | | |
| | (trifluoromethyl)-1H- | | |
| | pyrazolo[3,4-c]pyridin-7- | | |
| | yl]propyl}acetamide | | |
| M5 | 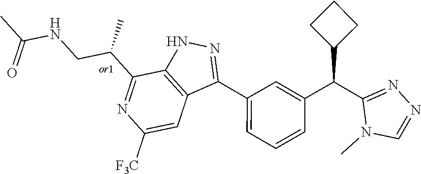 [SMILES: CC(=O)NC[C@H](C)c1nc(C(F)(F)F)cc2c(-c3cccc([C@H](c4nncn4C)C4CCC4)c3)n[nH]c12] | 512.2 | ¹H NMR (400 MHz, DMSO-d₆) δ 14.46 − 14.02 (m, 1H), 8.27 (s, 1H), 8.13 (s, 1H), 7.85 − 7.80 (m, 3H), 7.43 (t, J = 7.9 Hz, 1H), 7.27 (br d, J = 7.6 Hz, 1H), 4.30 (d, J = 10.5 Hz, 1H), 3.66 − 3.59 (m, 1H), 3.49 − 3.42 (m, 2H), 3.39 − 3.36 (m, 3H), 2.09 − 1.98 (m, 1H), 1.87 − 1.68 (m, 5H), 1.68 − 1.65 (m, 3H), 1.27 (d, J = 6.9 Hz, 3H). |
| | N-{(2ξ)-2-[3-{3-[(R)- | | |
| | cyclobutyl(4-methyl-4H-1,2,4- | | |
| | triazol-3-yl)methyl]phenyl}-5- | | |
| | (trifluoromethyl)- 1H- | | |
| | pyrazolo[3,4-c]pyridin-7- | | |
| | yl]propyl}acetamide | | |
| M6 | 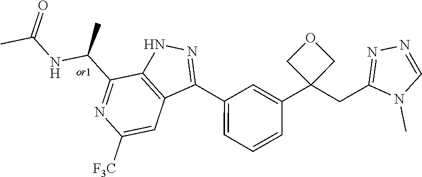 [SMILES: CC(=O)N[C@@H](C)c1nc(C(F)(F)F)cc2c(-c3cccc(C4(Cc5nncn5C)COC4)c3)n[nH]c12] | 500.2 | ¹H NMR (400 MHz, DMSO-d₆) δ 14.21 (br. s, 1H), 8.53 (d, J = 7.0 Hz, 1H), 8.20 (s, 1H), 8.18 (s, 1H), 7.89 (d, J = 7.8 Hz, 1H), 7.58 (t, J = 1.8 Hz, 1H), 7.48 (t, J = 7.7 Hz, 1H), 7.08 (br. d, J = 7.8 Hz, 1H), 5.55 − 5.47 (m, 1H), 5.00 (d, J = 6.1 Hz, 2H), 4.94 (dd, J = 6.1, 1.4 Hz, 2H), 3.57 (s, 2H), 2.96 (s, 3H), 1.88 (s, 3H), 1.50 (d, J = 7.0 Hz, 3H). |
| | N-{(1ξ)-1-[3-(3-{3-[(4-methyl- | | |
| | 4H-1,2,4-triazol-3- | | |
| | yl)methyl]oxetan-3-yl}phenyl)-5- | | |
| | (trifluoromethyl)-1H- | | |
| | pyrazolo[3,4-c]pyridin-7- | | |
| | yl]ethyl}acetamide | | |

### Example N1: 3-{3-[(2R)-1-(4-methyl-4H-1,2,4-triazol-3-yl)propan-2-yl]phenyl}-5-(trifluoromethyl)-1H-pyrazolo[3,4-c]pyridine

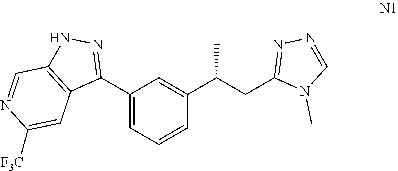 [SMILES: C[C@H](Cc1nncn1C)c1cccc(-c2n[nH]c3cnc(C(F)(F)F)cc23)c1]

Example N1 was prepared according to Method N.

### Step 1: (4R)-3-[(2E)-3-(3-bromophenyl)prop-2-enoyl]-4-phenyl-1,3-oxazolidin-2-one

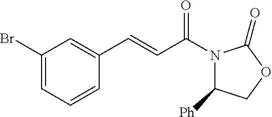 [SMILES: O=C(/C=C/c1cccc(Br)c1)N1C(=O)OC[C@H]1c1ccccc1]

A mixture of (2E)-3-(3-bromophenyl)prop-2-enoic acid (25.0 g, 110 mmol) and SOCl₂ (90 mL) was heated to 80° C. for 2 h. The mixture was concentrated under reduced pressure to give (2E)-3-(3-bromophenyl)prop-2-enoyl chloride. In a separate flask containing (4R)-4-phenyl-1,3-oxazolidin-2-one (17.9 g, 110 mmol) in THF (80 mL) at-70° C. under Ar was added LiHMDS (18.4 g, 110 mmol) dropwise and the reaction was allowed to stir for 30 min at −70° C. Then, (2E)-3-(3-bromophenyl)prop-2-enoyl chloride (27.0 g, 110 mmol) in THF (20 mL) was added dropwise at −70° C. and the reaction was allowed to stir for 2 h while warming to 0° C. The reaction was quenched with saturated aqueous ammonium chloride (100 mL) and extracted with EtOAc (2×100 mL). The combined organic layers were washed sequentially with water (2×100 mL), brine (50 mL), dried over Na₂SO₄, filtered, and concentrated under reduced pressure. The crude residue was purified by flash column chromatography (ISCO, SiO₂, pet ether/EtOAc 0-40%) followed by a second round of flash column chromatography (ISCO, SiO₂, pet ether/EtOAc 0-40%) to give (4R)-3-[(2E)-3-(3-bromophenyl)prop-2-enoyl]-4-phenyl-1,3-oxazolidin-2-one (18.3 g, 45%) as a light yellow solid. ¹H NMR (400 MHz, CDCl₃) δ 7.92 (d, J=15.6 Hz, 1H), 7.72 (s, 1H), 7.72-7.66 (m, 1H), 7.56-7.49 (m, 2H), 7.45-7.32 (m, 5H), 7.27-7.24 (m, 1H), 5.56 (dd, J=8.8, 3.9 Hz, 1H), 4.77 (t, J=8.8 Hz, 1H), 4.35 (dd, J=8.9, 3.9 Hz, 1H).

### Step 2: (4R)-3-[(3R)-3-(3-bromophenyl)butanoyl]-4-phenyl-1,3-oxazolidin-2-one

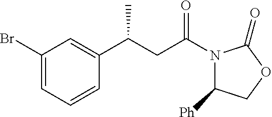 [SMILES: C[C@H](CC(=O)N1C(=O)OC[C@H]1c1ccccc1)c1cccc(Br)c1]

To a solution of copper(I) bromide-dimethyl sulfide complex (4.32 g, 21.0 mmol) in THF (30 mL) was added MeMgBr (14.0 mL, 42.0 mmol, 3.0 M) dropwise with stirring at −40° C. under nitrogen atmosphere. The mixture was allowed to warm to −20° C. for 40 min. Then the mixture was cooled to −40° C., and BF₃-Et₂₀ (2.98 g, 21.0 mmol) dropwise was added. The mixture was allowed to warm to −20° C. for 40 min. Again the mixture was cooled to −40° C., and a suspension of (4R)-3-[(2E)-3-(3-bromophenyl)prop-2-enoyl]-4-phenyl-1,3-oxazolidin-2-one (5.21 g, 13.99 mmol) in THF (30 mL) was added slowly with stirring at −40° C. Then the mixture was allowed to warm to −20° C. for 2 h. The reaction was then quenched with water, extracted with EtOAc (2×50 mL), washed with brine (50 mL), dried over Na₂SO₄, and concentrated under reduced pressure. The crude residue was purified by flash column chromatography (CombiFlash, 40 g SiO₂, EtOAc/pet ether 0-40%) to give (4R)-3-[(3R)-3-(3-bromophenyl)butanoyl]-4-phenyl-1,3-oxazolidin-2-one as a white solid (3.39 g, 64%). ¹H NMR (400 MHz, DMSO-d₆) δ 7.45 (s, 1H), 7.40-7.30 (m, 4H), 7.30-7.20 (m, 4H), 5.40 (dd, J=8.6, 3.7 Hz, 1H), 4.70 (t, J=8.7 Hz, 1H) 4.13 (dd, J=8.8, 3.8 Hz, 1H), 3.26-3.10 (m, 3H), 1.18 (d, J=6.1 Hz, 3H).

### Step 3: (3R)-3-(3-bromophenyl)butanehydrazide

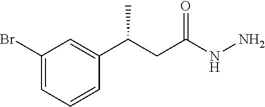

To (4R)-3-[(3R)-3-(3-bromophenyl)butanoyl]-4-phenyl-1,3-oxazolidin-2-one (3.70 g, 89.5 mmol) in THF (60 mL) was added hydrazine hydrate (5.62 g, 102 mmol) dropwise at 0° C. The mixture was stirred at 25° C. for 3 h, then concentrated under reduced pressure. The crude residue was purified using flash column chromatography (CombiFlash, 40 g SiO₂, EtOac/pet ether 0-100%) to give (3R)-3-(3-bromophenyl)butanehydrazide as a white gum (116 mg, 26%). LCMS (ESI+) 258.8 (M+H)⁺. ¹H NMR (400 MHz, DMSO-d₆) δ 8.95 (br s, 1H), 7.45-7.35 (m, 2H), 7.29-7.18 (m, 2H), 4.14 (br s, 2H), 3.22-3.14 (m, 1H), 2.37-2.22 (m, 2H), 1.18-1.14 (m, 3H).

### Step 4: 5-[(2R)-2-(3-bromophenyl)propyl]-4-methyl-4H-1,2,4-triazole-3-thiol

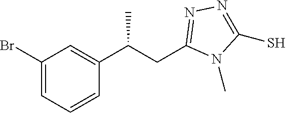 [SMILES: C[C@H](Cc1nnc(S)n1C)c1cccc(Br)c1]

To a solution of (3R)-3-(3-bromophenyl)butanehydrazide (2.56 g, 9.97 mmol) in THF (27.0 mL) was added methylamino(sulfanylidene)methane (1.46 g, 19.9 mmol) and was stirred at 20° C. for 2 h. To the reaction suspension was added EtOH (40 mL) and sodium hydroxide (3.98 g, 99.6 mmol, 5 M solution) at 15° C. The solution was stirred at room temperature for 16 h. The mixture was then diluted with ice water (50 mL) and 1 M HCl was added until pH ˜3. The mixture was extracted with EtOAc (2×30 mL), and the combined organic layers were washed with brine (30 mL), dried over Na₂SO₄, filtered, and concentrated under reduced pressure. The crude residue was purified by flash column chromatography (CombiFlash, 40 g SiO₂, EtOac/pet ether 0-50%) to give 5-[(2R)-2-(3-bromophenyl)propyl]-4-methyl-4H-1,2,4-triazole-3-thiol as a white solid (2.77 g, 91%). ¹H NMR (400 MHz, DMSO-d₆) δ 13.44 (s, 1H), 7.57-7.50 (m, 1H), 7.39 (dt, J=7.6, 1.6 Hz, 1H), 7.32-7.23 (m, 2H), 3.37 (s, 3H), 3.30-3.19 (m, 1H), 2.99 (d, J=7.2 Hz, 2H), 1.27 (d, J=6.9 Hz, 3H).

### Step 5: 3-[(2R)-2-(3-bromophenyl)propyl]-4-methyl-4H-1,2,4-triazole

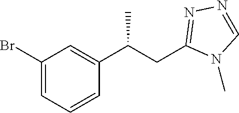 [SMILES: C[C@H](Cc1nncn1C)c1cccc(Br)c1]

To a solution of acetic acid (8 mL) in DCM (20 mL) was added hydrogen peroxide (5150 mg, 45.4 mmol) at 0° C. The resulting solution was added into a solution of 5-[(2R)-2-(3-bromophenyl)propyl]-4-methyl-4H-1,2,4-triazole-3-thiol (2765 mg, 9.084 mmol) in DCM (20 mL) at 0° C. and stirred for 2 h. The reaction solution was quenched with aqueous Na₂SO₃ and basified with NaHCO₃ to pH ˜8. The mixture was concentrated under reduced pressure, then extracted with EtOAc (3×50 mL). The combined organic layers were washed with brine (50 mL), dried over Na₂SO₄, filtered, and concentrated under reduced pressure. The residue was purified using flash column chromatography (ISCO, MeOH/DCM 0-10%) to afford 3-[(2R)-2-(3-bromophenyl)propyl]-4-methyl-4H-1,2,4-triazole as a colorless gum (1.83 g, 72%). ¹H NMR (400 MHz, DMSO-d₆) δ 8.29 (s, 1H), 7.49 (s, 1H), 7.39 (dt, J=7.4, 1.7 Hz, 1H), 7.30-7.21 (m, 2H), 3.47 (s, 3H), 3.29-3.23 (m, 1H), 2.98 (d, J=7.1 Hz, 2H), 1.26 (d, J=6.9 Hz, 3H).

### Step 6: 4-methyl-3-{(2R)-2-[3-(4,4,5,5-tetramethyl-1,3,2-dioxaborolan-2-yl)phenyl]propyl}-4H-1,2,4-triazole

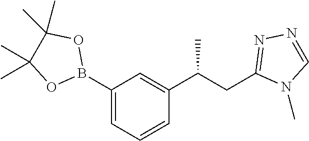 [SMILES: C[C@H](Cc1nncn1C)c1cccc(B2OC(C)(C)C(C)(C)O2)c1]

To a mixture of 3-[(2R)-2-(3-bromophenyl)propyl]-4-methyl-4H-1,2,4-triazole (300 mg, 1.07 mmol) and bis(pinacolato)diboron (326 mg, 1.28 mmol) in dioxane (10 mL) were added KOAc (315 mg, 3.21 mmol) and Pd(dppf)Cl₂ (78.4 mg, 0.107 mmol). The mixture was bubbled with N₂ for 1 min. and stirred at 90° C. for 16 h. The reaction mixture was then filtered and concentrated under reduced pressure to afford 4-methyl-3-{(2R)-2-[3-(4,4,5,5-tetramethyl-1,3,2-dioxaborolan-2-yl)phenyl]propyl}-4H-1,2,4-triazole (350 mg, quant. yield) which was carried forward without further purification. LCMS (ESI+) 328.3 (M+H)⁺. ¹H NMR (400 MHz, DMSO-d₆) δ 8.27 (s, 1H), 7.54 (s, 1H), 7.51 (d, J=7.1 Hz, 1H), 7.40 (br d, J=7.8 Hz, 1H), 7.34-7.27 (m, 1H), 3.44 (s, 3H), 3.27 (br s, 1H), 2.96 (d, J=7.5 Hz, 2H), 1.30 (s, 12H), 1.26 (d, J=6.9 Hz, 3H).

### Step 7: 3-{3-[(2R)-1-(4-methyl-4H-1,2,4-triazol-3-yl)propan-2-yl]phenyl}-5-(trifluoromethyl)-1H-pyrazolo[3,4-c]pyridine (Example N1)

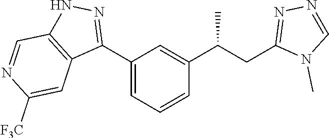 [SMILES: C[C@H](Cc1nncn1C)c1cccc(-c2n[nH]c3cnc(C(F)(F)F)cc23)c1]

To a mixture of tert-butyl 3-bromo-5-(trifluoromethyl)-1H-pyrazolo[3,4-c]pyridine-1-carboxylate (Intermediate 31) (329 mg, 1.07 mmol), 4-methyl-3-{(2R)-2-[3-(4,4,5,5-tetramethyl-1,3,2-dioxaborolan-2-yl)phenyl]propyl}-4H-1,2,4-triazole (350 mg, 1.07 mmol) in dioxane (5 mL) were added sodium carbonate (340 mg, 3.21 mmol), water (0.8 mL), and Pd(dppf)Cl₂ (78.3 mg, 0.107 mmol). The mixture was bubbled with N₂ for 1 min and stirred at 100° C. for 16 h. Then, the reaction was cooled, diluted with EtOAc (10 mL) and washed with water (10 mL). The layers were separated and the aqueous layer was extracted with EtOAc (10 mL). The organic layers were washed with brine (20 mL), dried over Na₂SO₄, filtered, and concentrated under reduced pressure. The crude residue was purified by flash column chromatography (ISCO, 8 g SiO₂, DCM/MeOH 0-10%) followed by preparative HPLC (Boston Prime C18 150×30 mm, 5 um column Mobile phase A: Water+Ammonium Hydroxide Mobile phase B: Acetonitrile 20-50% B in 9.0 minutes, 30 mL/min) to give 3-{3-[(2R)-1-(4-methyl-4H-1,2,4-triazol-3-yl)propan-2-yl]phenyl}-5-(trifluoromethyl)-1H-pyrazolo[3,4-c]pyridine (Example N1) as a white solid (48.4 mg, 12%). LCMS (ESI+) 387.1 (M+H)⁺. ¹H NMR (400 MHz, DMSO-d₆) δ 14.27 (brs, 1H), 9.24 (s, 1H), 8.47 (s, 1H), 8.27 (s, 1H), 7.93-7.81 (m, 2H), 7.53-7.45 (m, 1H), 7.38 (d, J=7.7 Hz, 1H), 3.47 (s, 3H), 3.45-3.38 (m, 1H), 3.07 (d, J=7.5 Hz, 2H), 1.41-1.40 (m, 1H), 1.36 (d, J=7.0 Hz, 2H).

### Prophetic Deuterated Analogs (PDA)

The compounds shown in Tables 11-13 are prophetic deuterated analogs (PDA) of Examples D15/D16/D46/D47, D18/D19/D25/D26 and C16/F1 respectively. The PDAs are predicted based on the metabolic profiles of these Examples.

TABLE 11
PDA of Example D15/D16/C46/C47

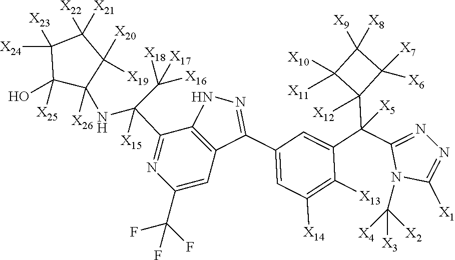

| Example | | | | | | | | | | | | | | | | | | | | | | | |
|---|---|---|---|---|---|---|---|---|---|---|---|---|---|---|---|---|---|---|---|---|---|---|---|
| NO. | X₁ | X₂ | X₃ | X₄ | X₅ | X₆ | X₇ | X₈ | X₉ | X₁₀ | X₁₁ | X₁₂ | X₁₃ | X₁₄ | X₁₅ | X₁₆ | X₁₇ | X₁₈ | X₁₉ | X₂₀ | X₂₁ | X₂₂ | X₂₃ |
| PDA-1 | D | H | H | H | H | H | H | H | H | H | H | H | H | H | H | H | H | H | H | H | H | H | H |
| PDA-2 | H | D | D | D | H | H | H | H | H | H | H | H | H | H | H | H | H | H | H | H | H | H | H |
| PDA-3 | H | H | H | H | D | H | H | H | H | H | H | H | H | H | H | H | H | H | H | H | H | H | H |
| PDA-4 | H | H | H | H | H | D | H | H | H | H | H | H | H | H | H | H | H | H | H | H | H | H | H |
| PDA-5 | H | H | H | H | H | H | D | H | H | H | H | H | H | H | H | H | H | H | H | H | H | H | H |
| PDA-6 | H | H | H | H | H | H | H | D | H | H | H | H | H | H | H | H | H | H | H | H | H | H | H |
| PDA-7 | H | H | H | H | H | H | H | H | D | H | H | H | H | H | H | H | H | H | H | H | H | H | H |
| PDA-8 | H | H | H | H | H | H | H | H | H | D | H | H | H | H | H | H | H | H | H | H | H | H | H |
| PDA-9 | H | H | H | H | H | H | H | H | H | H | D | H | H | H | H | H | H | H | H | H | H | H | H |
| PDA-10 | H | H | H | H | H | H | H | H | H | H | H | D | H | H | H | H | H | H | H | H | H | H | H |
| PDA-11 | H | H | H | H | H | H | H | H | H | H | H | H | D | H | H | H | H | H | H | H | H | H | H |
| PDA-12 | H | H | H | H | H | H | H | H | H | H | H | H | H | D | H | H | H | H | H | H | H | H | H |
| PDA-13 | H | H | H | H | H | H | H | H | H | H | H | H | H | H | D | H | H | H | H | H | H | H | H |
| PDA-14 | H | H | H | H | H | H | H | H | H | H | H | H | H | H | H | D | D | D | H | H | H | H | H |
| PDA-15 | H | H | H | H | H | H | H | H | H | H | H | H | H | H | H | H | H | H | D | H | H | H | H |
| PDA-16 | H | H | H | H | H | H | H | H | H | H | H | H | H | H | H | H | H | H | H | D | H | H | H |
| PDA-17 | H | H | H | H | H | H | H | H | H | H | H | H | H | H | H | H | H | H | H | H | D | H | H |
| PDA-18 | H | H | H | H | H | H | H | H | H | H | H | H | H | H | H | H | H | H | H | H | H | D | H |
| PDA-19 | H | H | H | H | H | H | H | H | H | H | H | H | H | H | H | H | H | H | H | H | H | H | D |
| PDA-20 | H | H | H | H | H | H | H | H | H | H | H | H | H | H | H | H | H | H | H | H | H | H | H |
| PDA-21 | H | H | H | H | H | H | H | H | H | H | H | H | H | H | H | H | H | H | H | H | H | H | H |
| PDA-22 | H | H | H | H | H | H | H | H | H | H | H | H | H | H | H | H | H | H | H | H | H | H | H |

PDA of Example D15/D16/C46/C47

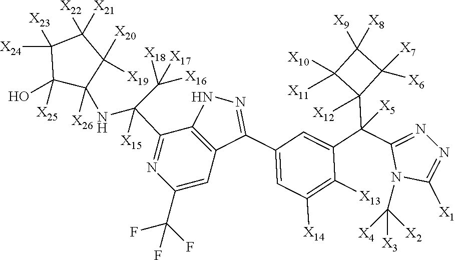

| Example | | | |
|---|---|---|---|
| NO. | X₂₄ | X₂₅ | X₂₆ |
| PDA-1 | H | H | H |
| PDA-2 | H | H | H |
| PDA-3 | H | H | H |
| PDA-4 | H | H | H |
| PDA-5 | H | H | H |
| PDA-6 | H | H | H |
| PDA-7 | H | H | H |
| PDA-8 | H | H | H |
| PDA-9 | H | H | H |
| PDA-10 | H | H | H |
| PDA-11 | H | H | H |
| PDA-12 | H | H | H |
| PDA-13 | H | H | H |
| PDA-14 | H | H | H |
| PDA-15 | H | H | H |
| PDA-16 | H | H | H |
| PDA-17 | H | H | H |
| PDA-18 | H | H | H |
| PDA-19 | H | H | H |
| PDA-20 | D | H | H |
| PDA-21 | H | D | H |
| PDA-22 | H | H | D |

TABLE 12
PDA of Example D18/D19/D25/D26

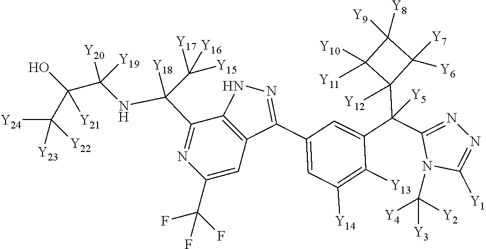

| Example | | | | | | | | | | | | | | | | | | | | | | | | |
|---|---|---|---|---|---|---|---|---|---|---|---|---|---|---|---|---|---|---|---|---|---|---|---|---|
| NO | Y₁ | Y₂ | Y₃ | Y₄ | Y₅ | Y₆ | Y₇ | Y₈ | Y₉ | Y₁₀ | Y₁₁ | Y₁₂ | Y₁₃ | Y₁₄ | Y₁₅ | Y₁₆ | Y₁₇ | Y₁₈ | Y₁₉ | Y₂₀ | Y₂₁ | Y₂₂ | Y₂₃ | Y₂₄ |
| PDA-23 | D | H | H | H | H | H | H | H | H | H | H | H | H | H | H | H | H | H | H | H | H | H | H | H |
| PDA-24 | H | D | D | D | H | H | H | H | H | H | H | H | H | H | H | H | H | H | H | H | H | H | H | H |
| PDA-25 | H | H | H | H | D | H | H | H | H | H | H | H | H | H | H | H | H | H | H | H | H | H | H | H |
| PDA-26 | H | H | H | H | H | D | H | H | H | H | H | H | H | H | H | H | H | H | H | H | H | H | H | H |
| PDA-27 | H | H | H | H | H | H | D | H | H | H | H | H | H | H | H | H | H | H | H | H | H | H | H | H |
| PDA-28 | H | H | H | H | H | H | H | D | H | H | H | H | H | H | H | H | H | H | H | H | H | H | H | H |
| PDA-29 | H | H | H | H | H | H | H | H | D | H | H | H | H | H | H | H | H | H | H | H | H | H | H | H |
| PDA-30 | H | H | H | H | H | H | H | H | H | D | H | H | H | H | H | H | H | H | H | H | H | H | H | H |
| PDA-31 | H | H | H | H | H | H | H | H | H | H | D | H | H | H | H | H | H | H | H | H | H | H | H | H |
| PDA-32 | H | H | H | H | H | H | H | H | H | H | H | D | H | H | H | H | H | H | H | H | H | H | H | H |
| PDA-33 | H | H | H | H | H | H | H | H | H | H | H | H | D | H | H | H | H | H | H | H | H | H | H | H |
| PDA-34 | H | H | H | H | H | H | H | H | H | H | H | H | H | D | H | H | H | H | H | H | H | H | H | H |
| PDA-35 | H | H | H | H | H | H | H | H | H | H | H | H | H | H | D | H | H | H | H | H | H | H | H | H |
| PDA-36 | H | H | H | H | H | H | H | H | H | H | H | H | H | H | H | D | D | H | H | H | H | H | H | H |
| PDA-37 | H | H | H | H | H | H | H | H | H | H | H | H | H | H | H | H | H | D | H | H | H | H | H | H |
| PDA-38 | H | H | H | H | H | H | H | H | H | H | H | H | H | H | H | H | H | H | D | H | H | H | H | H |
| PDA-39 | H | H | H | H | H | H | H | H | H | H | H | H | H | H | H | H | H | H | H | D | H | H | H | H |
| PDA-40 | H | H | H | H | H | H | H | H | H | H | H | H | H | H | H | H | H | H | H | H | D | H | H | H |
| PDA-41 | H | H | H | H | H | H | H | H | H | H | H | H | H | H | H | H | H | H | H | H | H | D | H | H |
| PDA-42 | H | H | H | H | H | H | H | H | H | H | H | H | H | H | H | H | H | H | H | H | H | H | D | H |
| PDA-43 | H | H | H | H | H | H | H | H | H | H | H | H | H | H | H | H | H | H | H | H | H | H | H | D |

TABLE 13
PDA of Example C16/F1

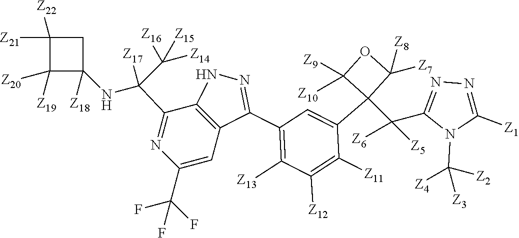

| Example | | | | | | | | | | | | | | | | | | | | | | |
|---|---|---|---|---|---|---|---|---|---|---|---|---|---|---|---|---|---|---|---|---|---|---|
| NO. | Z₁ | Z₂ | Z₃ | Z₄ | Z₅ | Z₆ | Z₇ | Z₈ | Z₉ | Z₁₀ | Z₁₁ | Z₁₂ | Z₁₃ | Z₁₄ | Z₁₅ | Z₁₆ | Z₁₇ | Z₁₈ | Z₁₉ | Z₂₀ | Z₂₁ | Z₂₂ |
| PDA-44 | D | H | H | H | H | H | H | H | H | H | H | H | H | H | H | H | H | H | H | H | H | H |
| PDA-45 | H | D | D | D | H | H | H | H | H | H | H | H | H | H | H | H | H | H | H | H | H | H |
| PDA-46 | H | H | H | H | D | H | H | H | H | H | H | H | H | H | H | H | H | H | H | H | H | H |
| PDA-47 | H | H | H | H | H | D | H | H | H | H | H | H | H | H | H | H | H | H | H | H | H | H |
| PDA-48 | H | H | H | H | H | H | D | H | H | H | H | H | H | H | H | H | H | H | H | H | H | H |
| PDA-49 | H | H | H | H | H | H | H | D | H | H | H | H | H | H | H | H | H | H | H | H | H | H |
| PDA-50 | H | H | H | H | H | H | H | H | D | H | H | H | H | H | H | H | H | H | H | H | H | H |
| PDA-51 | H | H | H | H | H | H | H | H | H | D | H | H | H | H | H | H | H | H | H | H | H | H |
| PDA-52 | H | H | H | H | H | H | H | H | H | H | D | H | H | H | H | H | H | H | H | H | H | H |
| PDA-53 | H | H | H | H | H | H | H | H | H | H | H | D | H | H | H | H | H | H | H | H | H | H |
| PDA-54 | H | H | H | H | H | H | H | H | H | H | H | H | D | H | H | H | H | H | H | H | H | H |
| PDA-55 | H | H | H | H | H | H | H | H | H | H | H | H | H | D | D | D | H | H | H | H | H | H |
| PDA-56 | H | H | H | H | H | H | H | H | H | H | H | H | H | H | H | H | D | H | H | H | H | H |
| PDA-57 | H | H | H | H | H | H | H | H | H | H | H | H | H | H | H | H | H | D | H | H | H | H |
| PDA-58 | H | H | H | H | H | H | H | H | H | H | H | H | H | H | H | H | H | H | D | H | H | H |
| PDA-59 | H | H | H | H | H | H | H | H | H | H | H | H | H | H | H | H | H | H | H | D | H | H |
| PDA-60 | H | H | H | H | H | H | H | H | H | H | H | H | H | H | H | H | H | H | H | H | D | H |
| PDA-61 | H | H | H | H | H | H | H | H | H | H | H | H | H | H | H | H | H | H | H | H | H | D |

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.org); King, R., “Biotransformations in Drug Metabolism,” Ch. 3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransformer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (Ihasalimited.org/products/meteor-nexus.htm) offers prediction of metabolic pathways and metabolite structures using a range of machine learning models, which covers phase I and phase II biotransformations of small molecules.

PDA-1 to PDA-27 in Tables 11-13 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

A person with ordinary skill may make additional deuterated analogs of Example D15, D16/C46/C47, Example D18/D19/D25/D26, and Example C16/F1 with different combinations of X1-X12, Y1-Y14 and Z1-Z14 as provided in Table 11, Table 12, and Table 13 respectively. Such additional deuterated analogs may provide similar therapeutic advantages that may be achieved by the deuterated analogs.

### Biological Assays and Data

### Ligand Displacement Assay (LDA)

The purpose of the homogeneous time-resolved fluorescence energy transfer (HTRF) ligand displacement assay is to determine whether the compounds of the disclosure are competitive with a BODIPY-labeled compound; “fluorescent tracer” aka [6-cyclopropyl-5-(17-{5-[(3,5-dimethyl-2H-pyrrol-2-ylidene-KN)methyl]-1H-pyrrol-2-yl-κN}-15-oxo-5,8,11-trioxa-2,14-diazaheptadecan-1-yl)-N-(3-{3-[(4-methyl-4H-1,2,4-triazol-3-yl)methyl]oxetan-3-yl}phenyl)pyridine-2-carboxamidato](difluorido)boron and to assess CBL-B binding selectivity over C-CBL. The synthesis of this fluorescent tracer compound is described below.

Purified CBL-B (residues 36-427) and C-CBL (residues 46-435) proteins from baculovirus-infected insect cells were biotin labeled on their amino-terminal AviTag™ epitopes by the E. coli biotin ligase BirA to bind terbium-labeled streptavidin (SA-Tb). After optimizing the assays and determining KD values for the fluorescent tracer to CBL-B and C-CBL, compounds were tested using three-fold serial dilutions in duplicate from a top concentration of 40 mM with a final DMSO concentration of 2%. 4 nM final concentration of CBL-B or C-CBL and SA-Tb (0.5 nM) were added to prepared compound dilution plates and incubated at room temperature for 15 minutes prior to the addition of fluorescent tracer (CBL-B: 289 nM, C-CBL: 1.6 mM; 3× empirically determined KD) in 50 mM HEPES pH 7.5, 4 mM MgCl2, 100 mM NaCl, 1 mM DTT, 0.01% Triton X-100, and 0.01% BSA. After a 1-hour incubation at room temperature, emissions at 520 nm and 490 nm were measured where ratiometric reductions of the data for a given test compound were used to obtain Ki values from nonlinear regression analyses with respect to compound concentration and the Morrison equation for tight-binding ligands. Ligand Displacement Assay competitive binding data is provided in Table 14. The Ki (nM) values reported are the geometric means (GMean) of the number of tests conducted.

TABLE 14
LDA competitive binding assay results

| | CBL-B | | C-CBL | |
|---|---|---|---|---|
| EXAMPLE | Kᵢ (nM) | Number of | Kᵢ (nM) | Number of |
| NO. | GMean | Tests | GMean | Tests |
| A1 | 74.4 | 8 | 681.3 | 6 |
| A2 | 80.4 | 1 | 224.1 | 1 |
| A3 | 31.1 | 1 | 362.1 | 1 |
| A4 | 17.9 | 1 | 279.4 | 1 |
| A5 | 89.1 | 1 | 1063.4 | 1 |
| A6 | 51.5 | 2 | 525.3 | 2 |
| A7 | 83.8 | 2 | 766.5 | 2 |
| B1 | 62.7 | 2 | 283.5 | 2 |
| B2 | 398.4 | 2 | 2400.8 | 1 |
| B3 | 75.3 | 1 | 363.2 | 1 |
| B4 | 73.9 | 2 | 253.6 | 2 |
| B5 | 58.9 | 1 | 375.3 | 1 |
| B6 | 71.8 | 2 | 437.2 | 2 |
| B7 | 25.1 | 2 | 150.3 | 2 |
| B8 | 28.6 | 1 | 142.3 | 1 |
| B9 | 74.5 | 1 | 237.9 | 1 |
| B10 | 57.8 | 1 | 186.9 | 1 |
| C1 | 0.6 | 3 | 7.1 | 3 |
| C2 | 3.9 | 6 | 55.6 | 6 |
| C3 | 7.8 | 2 | 52.1 | 2 |
| C4 | 3.3 | 1 | 47.4 | 1 |
| C5 | 55.3 | 1 | 525.7 | 1 |
| C6 | 25.8 | 1 | 256.0 | 1 |
| C7 | 3.7 | 5 | 53.1 | 5 |
| C8 | 1.1 | 5 | 3.0 | 5 |
| C9 | 5.0 | 3 | 85.2 | 3 |
| C10 | 11.4 | 1 | 108.1 | 1 |
| C11 | 0.6 | 1 | 3.7 | 1 |
| C12 | 0.9 | 2 | 1.3 | 2 |
| C13 | 2.3 | 2 | 8.0 | 2 |
| C14 | 0.4 | 1 | 2.3 | 1 |
| C15 | 8.9 | 2 | 42.1 | 2 |
| C16 | 10.0 | 2 | 49.3 | 2 |
| C17 | 4.0 | 2 | 24.8 | 2 |
| C18 | 2.2 | 2 | 15.1 | 2 |
| C19 | 10.8 | 1 | 61.3 | 1 |
| C20 | 2.4 | 1 | 16.9 | 1 |
| C21 | 21.3 | 1 | 93.5 | 1 |
| C22 | 24.1 | 1 | 133.3 | 1 |
| C23 | 5.7 | 1 | 24.8 | 1 |
| C24 | 28.3 | 1 | 133.6 | 1 |
| C25 | 6.5 | 1 | 33.1 | 1 |
| C26 | 40.5 | 1 | 198.2 | 1 |
| C27 | 31.4 | 2 | 76.0 | 2 |
| C28 | 33.4 | 2 | 114.0 | 2 |
| C29 | 8.5 | 4 | 46.3 | 4 |
| C30 | 3.6 | 6 | 23.4 | 6 |
| C31 | 21.7 | 2 | 111.6 | 2 |
| C32 | 6.7 | 4 | 55.6 | 4 |
| C33 | 22.6 | 2 | 110.6 | 2 |
| C34 | 8.9 | 2 | 88.5 | 2 |
| C35 | 3.1 | 3 | 29.1 | 3 |
| C36 | 1.8 | 3 | 16.8 | 3 |
| C37 | 5.3 | 2 | 50.6 | 2 |
| C38 | 6.9 | 4 | 32.9 | 3 |
| C39 | 7.4 | 2 | 73.2 | 2 |
| C40 | 7.1 | 2 | 95.9 | 2 |
| C41 | 3.2 | 1 | 30.1 | 1 |
| C42 | 2.5 | 1 | 28.2 | 1 |
| C43 | 6.7 | 1 | 87.7 | 1 |
| C44 | 2.2 | 1 | 25.0 | 1 |
| C45 | 2.5 | 1 | 22.1 | 1 |
| C46 | 9.4 | 2 | 67.8 | 2 |
| C47 | 0.7 | 8 | 5.0 | 8 |
| C48 | 2.5 | 1 | 33.0 | 1 |
| C49 | 2.1 | 1 | 20.9 | 1 |
| C50 | 24.7 | 6 | 90.0 | 4 |
| C51 | 22.0 | 3 | 115.2 | 1 |
| C52 | 32.9 | 3 | 133.8 | 2 |
| C53 | 66.6 | 2 | 311.8 | 2 |
| C54 | 10.7 | 2 | 49.4 | 2 |
| C55 | 21.2 | 2 | 65.3 | 2 |
| C56 | 43.6 | 1 | 200.8 | 1 |
| C57 | 18.9 | 1 | 96.3 | 1 |
| C58 | 14.8 | 1 | 82.4 | 1 |
| C59 | 45.2 | 1 | 171.1 | 1 |
| C60 | 10.7 | 1 | 57.5 | 1 |
| C61 | 53.0 | 1 | 232.0 | 1 |
| C62 | 34.3 | 1 | 161.7 | 1 |
| C63 | 27.0 | 1 | 120.4 | 1 |
| C64 | 31.2 | 1 | 117.3 | 1 |
| C65 | 21.8 | 1 | 126.2 | 1 |
| C66 | 20.0 | 1 | 111.8 | 1 |
| C67 | 12.6 | 1 | 72.1 | 1 |
| C68 | 24.8 | 1 | 123.7 | 1 |
| C69 | 34.5 | 1 | 184.6 | 1 |
| C70 | 20.6 | 1 | 112.8 | 1 |
| C71 | 25.5 | 1 | 124.5 | 1 |
| C72 | 18.6 | 1 | 83.3 | 1 |
| C73 | 19.3 | 1 | 111.4 | 1 |
| C74 | 7.5 | 2 | 83.1 | 2 |
| C75 | 5.0 | 2 | 54.8 | 2 |
| C76 | 3.3 | 2 | 41.4 | 2 |
| C77 | 2.4 | 2 | 29.5 | 2 |
| C78 | 56.6 | 1 | 253.2 | 1 |
| C79 | 20.4 | 1 | 94.1 | 1 |
| C80 | 10.1 | 2 | 71.5 | 2 |
| C81 | 6.5 | 2 | 18.2 | 2 |
| C82 | 5.2 | 3 | 23.3 | 3 |
| C83 | 37.1 | 1 | 136.7 | 1 |
| C84 | 25.1 | 1 | 144.0 | 1 |
| C85 | 15.2 | 1 | 92.5 | 1 |
| C86 | 9.0 | 2 | 49.4 | 2 |
| C87 | 21.2 | 2 | 209.6 | 2 |
| C88 | 7.2 | 1 | 132.1 | 1 |
| C89 | 8.0 | 1 | 105.1 | 1 |
| C90 | 9.4 | 1 | 93.3 | 1 |
| C91 | 12.7 | 2 | 105.1 | 2 |
| C92 | 4.4 | 2 | 50.0 | 2 |
| C93 | 3.6 | 1 | 15.9 | 1 |
| C94 | 2.8 | 2 | 14.9 | 2 |
| C95 | 4.7 | 1 | 28.0 | 1 |
| C96 | 9.2 | 1 | 53.5 | 1 |
| C97 | 31.8 | 1 | 141.2 | 1 |
| C98 | 26.9 | 1 | 109.2 | 1 |
| C99 | 3.1 | 1 | 13.8 | 1 |
| C100 | 2.8 | 1 | 40.4 | 1 |
| C101 | 2.5 | 1 | 21.0 | 1 |
| C102 | 10.7 | 2 | 113.1 | 1 |
| C103 | 46.0 | 1 | 388.9 | 1 |
| C104 | 28.0 | 1 | 217.8 | 1 |
| C105 | 29.4 | 1 | 243.6 | 1 |
| C106 | 41.6 | 1 | 475.2 | 1 |
| C107 | 33.9 | 1 | 363.3 | 1 |
| C108 | 79.4 | 1 | 630.0 | 1 |
| C109 | 48.8 | 1 | 442.2 | 1 |
| C110 | 50.0 | 1 | 464.2 | 1 |
| C111 | 26.9 | 5 | 313.1 | 5 |
| C112 | 15.4 | 2 | 114.5 | 2 |
| C113 | 27.1 | 1 | 232.8 | 1 |
| C114 | 87.9 | 1 | 1069.2 | 1 |
| C115 | 73.8 | 1 | 1020.2 | 1 |
| C116 | 18.2 | 1 | 203.7 | 1 |
| C117 | 7.9 | 1 | 83.6 | 1 |
| C118 | 2.1 | 2 | 25.5 | 2 |
| C119 | 28.7 | 1 | 220.4 | 1 |
| C120 | 13.6 | 1 | 105.1 | 1 |
| C121 | 18.9 | 1 | 111.1 | 1 |
| C122 | 29.2 | 1 | 293.7 | 1 |
| C123 | 19.5 | 1 | 188.6 | 1 |
| C124 | 7.9 | 1 | 109.3 | 1 |
| C125 | 12.2 | 1 | 146.9 | 1 |
| D1 | 22.4 | 7 | 210.5 | 7 |
| D2 | 5.8 | 4 | 77.1 | 4 |
| D3 | 6.0 | 3 | 69.7 | 3 |
| D4 | 5.1 | 3 | 54.8 | 3 |
| D5 | 32.7 | 1 | 148.4 | 1 |
| D6 | 4.6 | 1 | 21.0 | 1 |
| D7 | 7.4 | 1 | 28.1 | 1 |
| D8 | 25.4 | 1 | 113.4 | 1 |
| D9 | 17.4 | 1 | 71.8 | 1 |
| D10 | 6.6 | 1 | 25.0 | 1 |
| D11 | 7.4 | 1 | 27.2 | 1 |
| D12 | 15.3 | 1 | 52.9 | 1 |
| D13 | 15.0 | 1 | 53.6 | 1 |
| D14 | 42.9 | 1 | 183.2 | 1 |
| D15 | 0.9 | 5 | 1.0 | 5 |
| D16 | 2.5 | 6 | 24.5 | 6 |
| D17 | 1.4 | 1 | 33.2 | 1 |
| D18 | 1.0 | 8 | 9.0 | 8 |
| D19 | 1.0 | 7 | 12.4 | 7 |
| D20 | 1.7 | 2 | 2.3 | 2 |
| D21 | 5.4 | 2 | 68.1 | 2 |
| D22 | 0.8 | 3 | 5.8 | 3 |
| D23 | 1.8 | 2 | 9.9 | 2 |
| D24 | 2.2 | 2 | 22.3 | 2 |
| D25 | 6.4 | 2 | 51.1 | 2 |
| D26 | 1.8 | 2 | 14.0 | 2 |
| D27 | 1.0 | 4 | 9.1 | 4 |
| D28 | 0.7 | 2 | 0.6 | 2 |
| D29 | 1.9 | 2 | 23.7 | 2 |
| D30 | 0.6 | 1 | 5.1 | 1 |
| D31 | 4.0 | 1 | 27.3 | 1 |
| D32 | 1.3 | 1 | 10.2 | 1 |
| D34 | 6.5 | 1 | 59.9 | 1 |
| D35 | 12.9 | 1 | 100.1 | 1 |
| D36 | 1.4 | 1 | 16.4 | 1 |
| D37 | 22.2 | 1 | 64.5 | 1 |
| D38 | 34.0 | 1 | 135.2 | 1 |
| D39 | 42.1 | 1 | 228.8 | 1 |
| D40 | 23.9 | 1 | 98.1 | 1 |
| D41 | 18.5 | 1 | 65.5 | 1 |
| D42 | 35.3 | 1 | 163.2 | 1 |
| D43 | 14.5 | 2 | 75.1 | 2 |
| D44 | 15.6 | 1 | 47.4 | 1 |
| D45 | 19.9 | 2 | 80.3 | 2 |
| D46 | 46.5 | 1 | 249.5 | 1 |
| D47 | 36.9 | 1 | 161.5 | 1 |
| D48 | 16.1 | 1 | 44.9 | 1 |
| D49 | 17.8 | 1 | 103.5 | 1 |
| D50 | 9.0 | 1 | 30.7 | 1 |
| D51 | 5.7 | 1 | 19.6 | 1 |
| D52 | 31.9 | 1 | 117.6 | 1 |
| D53 | 11.8 | 1 | 36.9 | 1 |
| D54 | 20.9 | 1 | 80.0 | 1 |
| D55 | 4.8 | 1 | 76.5 | 1 |
| D56 | 6.2 | 2 | 62.3 | 2 |
| D57 | 5.2 | 1 | 46.7 | 1 |
| D58 | 14.3 | 1 | 172.7 | 1 |
| D59 | 2.6 | 1 | 26.6 | 1 |
| D60 | 12.3 | 1 | 82.3 | 1 |
| D61 | 22.0 | 1 | 144.8 | 1 |
| D62 | 4.3 | 1 | 39.8 | 1 |
| D63 | 8.4 | 3 | 67.0 | 3 |
| D64 | 8.7 | 1 | 58.0 | 1 |
| D65 | 13.7 | 1 | 109.8 | 1 |
| D66 | 2.6 | 1 | 22.1 | 1 |
| D67 | 10.9 | 1 | 78.5 | 1 |
| D68 | 15.2 | 1 | 133.6 | 1 |
| D69 | 9.3 | 1 | 54.3 | 1 |
| D70 | 7.1 | 1 | 48.4 | 1 |
| D71 | 1.3 | 1 | 13.0 | 1 |
| D72 | 8.7 | 1 | 116.2 | 1 |
| D73 | 12.1 | 1 | 157.1 | 1 |
| D74 | 31.5 | 1 | 365.8 | 1 |
| D75 | 11.0 | 1 | 127.8 | 1 |
| D76 | 0.7 | 1 | 2.9 | 1 |
| D77 | 0.8 | 1 | 4.5 | 1 |
| D78 | 71.2 | 1 | 270.6 | 1 |
| D79 | 295.5 | 1 | 1163.4 | 1 |
| D80 | 23.0 | 1 | 180.6 | 1 |
| D81 | 25.7 | 1 | 191.2 | 1 |
| D82 | 32.5 | 1 | 257.7 | 1 |
| D83 | 27.7 | 1 | 198.4 | 1 |
| D84 | 21.9 | 1 | 143.6 | 1 |
| D85 | 23.4 | 1 | 237.3 | 1 |
| D86 | 16.1 | 1 | 106.0 | 1 |
| D87 | 12.2 | 1 | 111.7 | 1 |
| D88 | 3.9 | 1 | 35.2 | 1 |
| D89 | 20.9 | 1 | 290.5 | 1 |
| D90 | 8.9 | 1 | 146.6 | 1 |
| D91 | 6.2 | 1 | 66.8 | 1 |
| D92 | 4.5 | 1 | 36.1 | 1 |
| D93 | 0.8 | 1 | 2.4 | 1 |
| D94 | 29.2 | 1 | 192.0 | 1 |
| D95 | 40.7 | 1 | 360.2 | 1 |
| D96 | 41.9 | 1 | 377.4 | 1 |
| D97 | 25.0 | 2 | 139.5 | 2 |
| D98 | 25.5 | 2 | 110.0 | 2 |
| E1 | 268.3 | 1 | 2322.7 | 1 |
| E2 | 244.4 | 1 | 2650.7 | 1 |
| F1 | 1.3 | 54 | 7.3 | 54 |
| G1 | 11.0 | 2 | 118.1 | 2 |
| H1 | 4.3 | 1 | 48.4 | 1 |
| H2 | 1.4 | 1 | 18.3 | 1 |
| H3 | 8.1 | 1 | 28.8 | 1 |
| H4 | 1.0 | 1 | 4.5 | 1 |
| H5 | 28.5 | 1 | 93.6 | 1 |
| H6 | 35.6 | 1 | 142.5 | 1 |
| I1 | 0.8 | 1 | 1.9 | 1 |
| I2 | 7.8 | 1 | 64.5 | 1 |
| I3 | 0.8 | 1 | 0.7 | 1 |
| I4 | 3.4 | 1 | 24.3 | 1 |
| I5 | 2.7 | 1 | 22.9 | 1 |
| I6 | 7.8 | 1 | 84.3 | 1 |
| J1 | 13.5 | 1 | 58.1 | 1 |
| J2 | 19.1 | 1 | 76.6 | 1 |
| J3 | 16.7 | 1 | 76.3 | 1 |
| J4 | 9.2 | 1 | 83.6 | 1 |
| J5 | 10.0 | 1 | 87.1 | 1 |
| J6 | 48.9 | 1 | 196.5 | 1 |
| J7 | 92.0 | 1 | 420.0 | 1 |
| J8 | 69.3 | 4 | 250.8 | 2 |
| J9 | 92.2 | 2 | 228.4 | 2 |
| J10 | 25.1 | 1 | 91.9 | 1 |
| J11 | 41.6 | 1 | 275.4 | 1 |
| J12 | 16.1 | 1 | 117.8 | 1 |
| K1 | 36.0 | 1 | 289.4 | 1 |
| K2 | 48.6 | 1 | 286.6 | 1 |
| K3 | 11.0 | 1 | 96.5 | 1 |
| K4 | 17.6 | 1 | 156.2 | 1 |
| K5 | 55.3 | 3 | 199.0 | 3 |
| K6 | 26.5 | 3 | 78.6 | 3 |
| K7 | 26.6 | 3 | 97.8 | 3 |
| K8 | 33.4 | 2 | 133.2 | 2 |
| K9 | 45.4 | 1 | 328.3 | 1 |
| K10 | 45.1 | 2 | 218.6 | 1 |
| K11 | 31.2 | 2 | 119.0 | 1 |
| K12 | 19.3 | 5 | 135.5 | 5 |
| K13 | 27.0 | 1 | 214.8 | 1 |
| K14 | 24.8 | 2 | 194.9 | 2 |
| K15 | 30.1 | 2 | 263.4 | 2 |
| K16 | 32.0 | 2 | 140.4 | 2 |
| K17 | 44.6 | 2 | 144.8 | 2 |
| L1 | 74.2 | 1 | 439.1 | 1 |
| L2 | 90.1 | 2 | 721.4 | 2 |
| L3 | 20.1 | 1 | 159.5 | 1 |
| L4 | 19.2 | 1 | 178.0 | 1 |
| M1 | 26.3 | 2 | 79.4 | 2 |
| M2 | 30.9 | 2 | 224.7 | 2 |
| M3 | 29.0 | 2 | 214.0 | 2 |
| M4 | 20.5 | 2 | 157.2 | 2 |
| M5 | 52.5 | 2 | 527.3 | 2 |
| M6 | 19.5 | 1 | 68.6 | 1 |
| N1 | 70.0 | 2 | 248.8 | 2 |

### Preparation of [6-cyclopropyl-5-(17-{5-[(3,5-dimethyl-2H-pyrrol-2-ylidene-κN)methyl]-1H-pyrrol-2-yl-κN}-15-oxo-5,8,11-trioxa-2,14-diazaheptadecan-1-yl)-N-(3-{3-[(4-methyl-4H-1,2,4-triazol-3-yl)methyl]oxetan-3-yl}phenyl)pyridine-2-carboxamidato](difluorido)boron

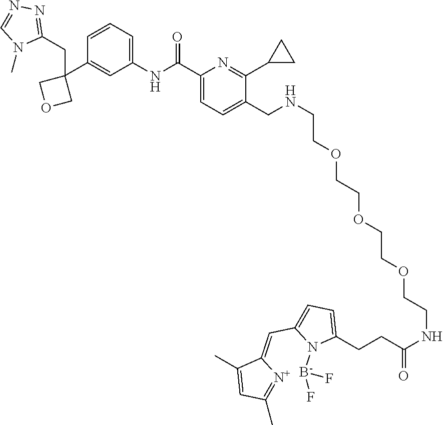

### Synthesis of 3-{3-[(4-methyl-4H-1,2,4-triazol-3-yl)methyl]oxetan-3-yl}aniline

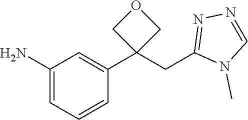 [SMILES: Cn1cnnc1CC1(c2cccc(N)c2)COC1]

Step 1: Ethyl (3-{3-[(tert-butoxycarbonyl)amino]phenyl}oxetan-3-yl)acetate

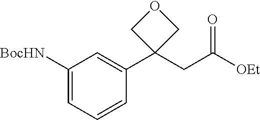 [SMILES: CCOC(=O)CC1(c2cccc(NC(=O)OC(C)(C)C)c2)COC1]

To a yellow solution of chloro(1,5-cyclooctadiene)rhodium(I) dimer (832 mg, 1.69 mmol) in 1,4-dioxane (150 mL) was added 1.67 M aq KOH (3550 mg, 63.3 mmol) and degassed with N₂ 4 times. Then, a solution of {3-[(tert-butoxycarbonyl)amino]phenyl}boronic acid (18.0 g 76.0 mmol) and ethyl (oxetan-3-ylidene)acetate (6.0 g, 42.2 mmol) in 1,4-dioxane (45 mL) which was degassed with N₂ for 4 times was added at 25° C. and stirred at 25° C. under Ar for 16 h. The mixture was diluted with saturated aqueous NH₄Cl (200 mL) and extracted with EtOAc (2×200 mL) then the extract was washed with brine (100 mL), dried over Na₂SO₄ and concentrated in vacuo. The crude residue was purified by flash column chromatography (80 g SiO₂, pet ether/EtOAc 0-30%) to afford ethyl (3-{3-[(tert-butoxycarbonyl)-amino]phenyl}oxetan-3-yl)acetate as yellow oil (12 g, 85%). ¹H NMR (400 MHz, CDCl₃) δ 7.26-7.16 (m, 3H), 6.83 (d, J=7.3 Hz, 1H), 6.53 (br s, 1H), 5.01 (d, J=6.1 Hz, 2H), 4.85 (d, J=6.1 Hz, 2H), 4.02 (q, J=7.1 Hz, 2H), 3.11 (s, 2H), 1.52 (s, 9H), 1.14 (t, J=7.1 Hz, 3H).

### Step 2: tert-butyl (3-(3-(2-hydrazineyl-2-oxoethyl)oxetan-3-yl)phenyl)carbamate

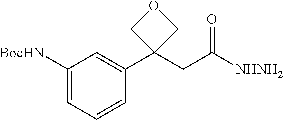 [SMILES: CC(C)(C)OC(=O)Nc1cccc(C2(CC(=O)NN)COC2)c1]

A solution of ethyl (3-{3-[(tert-butoxycarbonyl)amino]phenyl}oxetan-3-yl)acetate (12.0 g, 35.8 mmol) and hydrazine monohydrate (17.9 g, 358 mmol) in EtOH (120 mL) was stirred at 80° C. for 18 h. The mixture was concentrated to dryness and co-evaporated with EtOH until solid was obtained to afford crude tert-butyl (3-(3-(2-hydrazineyl-2-oxoethyl)oxetan-3-yl)phenyl)carbamate as yellow solid (12 g, 104%) which was taken as is into the next step.

### Step 3: tert-butyl (3-{3-[(4-methyl-5-sulfanyl-4H-1,2,4-triazol-3-yl)methyl]oxetan-3-yl}phenyl)carbamate

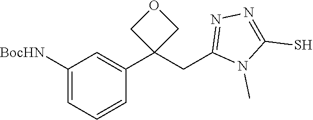 [SMILES: Cn1c(S)nnc1CC1(c2cccc(NC(=O)OC(C)(C)C)c2)COC1]

To a suspension of tert-butyl {3-[3-(2-hydrazineyl-2-oxoethyl)oxetan-3-yl]phenyl}carbamate (11.5 g, 35.8 mmol) in EtOH (150 mL) was added methyl isothiocyanate (5.2 g, 71.1 mmol) in one portion at 25° C. and was stirred at 25° C. for 50 h. Then 5M NaOH (14.3 g, 358 mmol) was added at 15° C. which turned to a clear yellow solution which was stirred for 48 h. The mixture was diluted with ice water (150 mL) and adjusted with 1N HCl until pH=3. The mixture was extracted with EtOAc (2×300 mL). The combined organic layers were washed with brine (300 mL), dried over Na₂SO₄, filtered, and concentrated under vacuum to give the crude residue which was crystallized from ˜EtOAc/pet ether (30 mL/100 mL) to collect a slight yellow solid and the filtrate was further purified below. This yellow solid was stirred with EtOAc (50 mL) and kept standing at rt overnight, the solid was filtered off and the filtrate concentrated to give tert-butyl (3-{3-[(4-methyl-5-sulfanyl-4H-1,2,4-triazol-3-yl)methyl]oxetan-3-yl}phenyl)carbamate as a yellow solid (3 g, 22%). The filtrate from the above was concentrated and purified via flash column chromatography (40 g SiO₂, pet ether/EtOAc 0-100%) to afford additional tert-butyl (3-{3-[(4-methyl-5-sulfanyl-4H-1,2,4-triazol-3-yl)methyl]oxetan-3-yl}phenyl)carbamate as a yellow solid (2.7 g, 20%). ¹H NMR (400 MHz, DMSO-d₆) δ 13.48 (s, 1H), 9.30 (s, 1H), 7.40-7.13 (m, 3H), 6.67 (d, J=7.7 Hz, 1H), 4.79 (q, J=6.1 Hz, 4H), 3.50-3.42 (m, 2H), 2.86 (s, 3H), 1.61-1.39 (m, 9H).

### Step 4: tert-butyl (3-{3-[(4-methyl-4H-1,2,4-triazol-3-yl)methyl]oxetan-3-yl}phenyl)carbamate

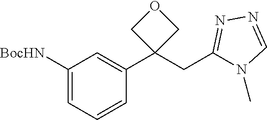 [SMILES: Cn1cnnc1CC1(c2cccc(NC(=O)OC(C)(C)C)c2)COC1]

To a solution of tert-butyl (3-{3-[(4-methyl-5-sulfanyl-4H-1,2,4-triazol-3-yl)methyl]oxetan-3-yl}phenyl)carbamate (5.90 g, 14.9 mmol) in DCM (50 mL) and AcOH (10 mL) was added hydrogen peroxide solution 30% (8.44 g, 74.4 mmol) at 0-10° C. in an ice bath. The reaction was stirred at 5° C. for 2 h. Then, the reaction solution was quenched by sat. aqueous Na₂SO₃ (50 mL) and then adjusted to pH=9 with K₂CO₃. The aqueous was extracted with DCM (2×100 mL) then EtOAc (100 mL), dried over Na₂SO₄, filtered, and concentrated in vacuo to give yellow solid. The residue was dissolved in ˜30 mL EtOAc and pet ether was added dropwise until mixture turned cloudy and allowed to stand at rt for 24 h. The mixture was filtered and the solid was dried in vacuo to afford tert-butyl (3-{3-[(4-methyl-4H-1,2,4-triazol-3-yl)methyl]oxetan-3-yl}phenyl)carbamate as a yellow solid. (3 g, 59%). LCMS (ESI+) 345.1 (M+H)⁺.

### Step 5: 3-{3-[(4-methyl-4H-1,2,4-triazol-3-yl)methyl]oxetan-3-yl}aniline

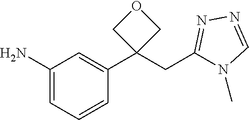 [SMILES: Cn1cnnc1CC1(c2cccc(N)c2)COC1]

To a solution of tert-butyl (3-{3-[(4-methyl-4H-1,2,4-triazol-3-yl)methyl]oxetan-3-yl}phenyl)carbamate (3.0 g, 8.71 mmol) in DCM (30 mL) was added TFA (15 mL) at 0-10° C. in an ice bath. The reaction was stirred at 25° C. for 2 h and the mixture was concentrated and co-evaporated with DCM in vacuo to afford crude residue which was dissolved in DCM (40 mL) and basified with NH₃/MeOH. The solution was concentrated with silica gel (10 g) and purified via flash column chromatography (24 g SiO₂, DCM MeOH 0-10%) to afford 3-{3-[(4-methyl-4H-1,2,4-triazol-3-yl)methyl]oxetan-3-yl}aniline as a yellow solid (1.5 g, 71%). ¹H NMR (400 MHz, DMSO-d₆) δ 8.20 (s, 1H), 6.90 (t, J=7.8 Hz, 1H), 6.41 (dd, J=1.3, 8.0 Hz, 1H), 6.05 (t, J=1.8 Hz, 1H), 5.95 (d, J=7.5 Hz, 1H), 5.02 (s, 2H), 4.85 (d, J=5.8 Hz, 2H), 4.75 (d, J=6.0 Hz, 2H), 3.39 (s, 2H), 2.83 (s, 3H). LCMS (ESI+) 245.3 (M+H)⁺.

### Synthesis of 5-(13-amino-5,8,11-trioxa-2-azatridecan-1-yl)-6-cyclopropyl-N-(3-{3-[(4-methyl-4H-1,2,4-triazol-3-yl)methyl]oxetan-3-yl}phenyl)pyridine-2-carboxamide

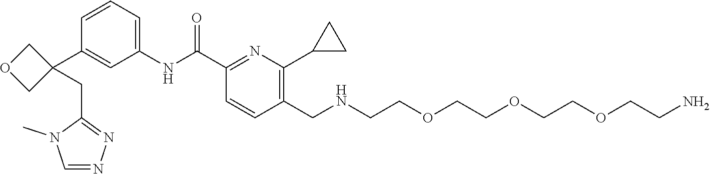 [SMILES: Cn1cnnc1CC1(c2cccc(NC(=O)c3ccc(CNCCOCCOCCOCCN)c(C4CC4)n3)c2)COC1]

### Step 1: Methyl 5-cyano-6-cyclopropylpyridine-2-carboxylate

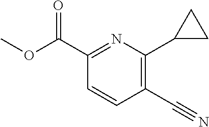

A mixture of methyl 6-chloro-5-cyanopyridine-2-carboxylate (525 mg, 2.67 mmol), cyclopropylboronic acid (688 mg, 8.01 mmol), K₂CO₃ (554 mg, 4.01 mmol), and Pd(dppf)Cl₂ (195 mg, 0.267 mmol) in 1,4-dioxane (10 mL) was degassed with N₂ for 1 min then stirred at 100° C. for 16 h. Then, the reaction mixture was concentrated under reduced pressure and purified by flash column chromatography (ISCO 20 g SiO₂, pet ether/EtOAc 0-20%) to give methyl 5-cyano-6-cyclopropylpyridine-2-carboxylate as an off-white solid (490 mg, 91%). ¹H NMR (DMSO-d₆, 500 MHz) δ 8.40 (d, J=8.1 Hz, 1H), 7.90 (d, J=7.9 Hz, 1H), 3.95-3.83 (m, 3H), 2.48-2.43 (m, 1H), 1.23-1.17 (m, 2H), 1.14-1.09 (m, 2H).

### Step 2: Methyl 5-{[(tert-butoxycarbonyl)amino]methyl}-6-cyclopropylpyridine-2-carboxylate

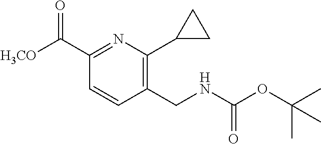 [SMILES: COC(=O)c1ccc(CNC(=O)OC(C)(C)C)c(C2CC2)n1]

A suspension of methyl 5-cyano-6-cyclopropylpyridine-2-carboxylate (190 mg, 0.940 mmol), Raney-Ni (110 mg, 1.88 mmol) and Boc₂O (205 mg, 0.940 mmol) in MeOH (20 mL) was degassed with H₂ (20 Psi) 5 times and stirred at 25° C. for 3 h. Then, the suspension was filtered, and the filtrate was concentrated in vacuo and purified by flash column chromatography (ISCO, 4 g SiO₂, pet ether/EtOAc 0-30%) to give methyl 5-{[(tert-butoxycarbonyl)amino]methyl}-6-cyclopropylpyridine-2-carboxylate as a colorless gum. (195 mg, 68%).

### Step 3: 5-{[(tert-butoxycarbonyl)amino]methyl}-6-cyclopropylpyridine-2-carboxylic acid

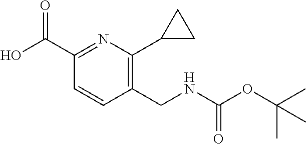 [SMILES: CC(C)(C)OC(=O)NCc1ccc(C(=O)O)nc1C1CC1]

To a solution of methyl 5-{[(tert-butoxycarbonyl)amino]methyl}-6-cyclopropylpyridine-2-carboxylate (195 mg, 0.637 mmol) in MeOH (3 mL) and H₂O (0.80 mL) was added LiOH—H₂O (53.4 mg, 1.27 mmol). The reaction was stirred at 25° C. for 16 h.

Then, the reaction was concentrated and dissolved in H₂O (5 mL), acidified to pH-1 with 1N HCl, and extracted with EA (3×10 mL). The combined organic layers were washed with brine (20 mL), dried over Na₂SO₄, filtered, and concentrated in vacuo to yield 5-{[(tert-butoxycarbonyl)amino]methyl}-6-cyclopropylpyridine-2-carboxylic acid as a colorless gum (180 mg, 97%). LCMS(ESI+) 293 (M+H)⁺.

### Step 4: tert-butyl ({2-cyclopropyl-6-[(3-{3-[(4-methyl-4H-1,2,4-triazol-3-yl)methyl]oxetan-3-yl}phenyl)carbamoyl]pyridin-3-yl}methyl)carbamate

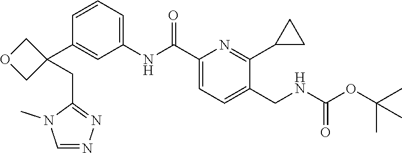 [SMILES: Cn1cnnc1CC1(c2cccc(NC(=O)c3ccc(CNC(=O)OC(C)(C)C)c(C4CC4)n3)c2)COC1]

To a solution of 5-{[(tert-butoxycarbonyl)amino]methyl}-6-cyclopropylpyridine-2-carboxylic acid (180 mg, 0.616 mmol) in DMF (3.5 mL) was added HATU (281 mg, 0.739 mmol) at 0° C. and stirred for 30 min. 3-{3-[(4-methyl-4H-1,2,4-triazol-3-yl)methyl]oxetan-3-yl}aniline (150 mg, 0.616 mmol) and DIPEA (239 mg, 1.85 mmol) were then added to above solution at 0° C. and stirred at 25° C. for 16 h. The reaction solution was diluted with H₂O (5 mL), brine (5 mL) and EtOAc (10 mL). The organic layer was separated, and the aqueous layer was extracted with EtOAc (2×10 mL). The combined organic layers were washed with sat. Na₂CO₃ (2×15 mL), brine (4×20 mL), dried over Na₂SO₄, filtered and concentrated in vacuo to yield crude product as a yellow solid, which was purified via preparative TLC (DCM:MeOH=10:1) to yield tert-butyl ({2-cyclopropyl-6-[(3-{3-[(4-methyl-4H-1,2,4-triazol-3-yl)methyl]oxetan-3-yl}phenyl)carbamoyl]pyridin-3-yl}methyl)carbamate as an off-white solid (300 mg, 94%). ¹H NMR (400 MHz, DMSO-d₆) δ 9.98 (s, 1H), 8.19 (s, 1H), 7.86 (d, J=7.9 Hz, 1H), 7.70 (dd, J=8.8, 11.3 Hz, 2H), 7.51 (br t, J=5.8 Hz, 1H), 7.41 (s, 1H), 7.27 (t, J=7.9 Hz, 1H), 6.65 (d, J=7.8 Hz, 1H), 4.94 (d, J=5.9 Hz, 2H), 4.86 (d, J=6.0 Hz, 2H), 4.39 (br d, J=5.8 Hz, 2H), 3.49 (s, 2H), 2.92 (s, 3H), 2.31-2.26 (m, 1H), 1.41 (s, 9H), 1.27-1.22 (m, 2H), 1.06-0.97 (m, 2H). LCMS (ESI+) 519.3 (M+H)⁺.

### Step 5: 5-(aminomethyl)-6-cyclopropyl-N-(3-{3-[(4-methyl-4H-1,2,4-triazol-3-yl)methyl]oxetan-3-yl}phenyl)pyridine-2-carboxamide

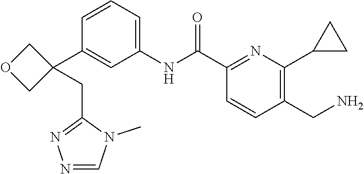 [SMILES: Cn1cnnc1CC1(c2cccc(NC(=O)c3ccc(CN)c(C4CC4)n3)c2)COC1]

To a solution of tert-butyl ({2-cyclopropyl-6-[(3-{3-[(4-methyl-4H-1,2,4-triazol-3-yl)methyl]oxetan-3-yl}phenyl)carbamoyl]pyridin-3-yl}methyl)carbamate (300 mg, 0.578 mmol) in DCM (10 mL) was added TFA (2 mL) and stirred at 20° C. for 40 min. The reaction mixture was concentrated and dissolved in H₂O (10 mL), basified to pH-8 by NH₃·H₂O, and extracted with DCM:MeOH=10:1 (3×10 mL). The combined organic layers were washed with brine (20 mL), dried with Na₂SO₄, filtered, and concentrated in vacuo to yield 5-(aminomethyl)-6-cyclopropyl-N-(3-{3-[(4-methyl-4H-1,2,4-triazol-3-yl)methyl]oxetan-3-yl}phenyl)pyridine-2-carboxamide as a yellow solid (210 mg, 87%). 1H NMR (400 MHz, DMSO-d₆) δ 9.98 (s, 1H), 8.20 (s, 1H), 7.96 (d, J=7.8 Hz, 1H), 7.86 (d, J=7.8 Hz, 1H), 7.69 (dd, J=1.1, 8.1 Hz, 1H), 7.41 (s, 1H), 7.27 (t, J=7.9 Hz, 1H), 6.64 (d, J=7.7 Hz, 1H), 4.94 (d, J=6.0 Hz, 2H), 4.86 (d, J=6.0 Hz, 2H), 3.99 (s, 2H), 3.49 (s, 2H), 2.92 (s, 3H), 2.31-2.24 (m, 1H), 2.01 (br d, J=18.6 Hz, 2H), 1.26-1.21 (m, 2H), 1.04-0.97 (m, 2H). LCMS (ESI+) 419.1 (M+H)⁺.

### Step 6: tert-butyl (1-{2-cyclopropyl-6-[(3-{3-[(4-methyl-4H-1,2,4-triazol-3-yl)methyl]oxetan-3-yl}phenyl)carbamoyl]pyridin-3-yl}-5,8,11-trioxa-2-azatridecan-13-yl)carbamate

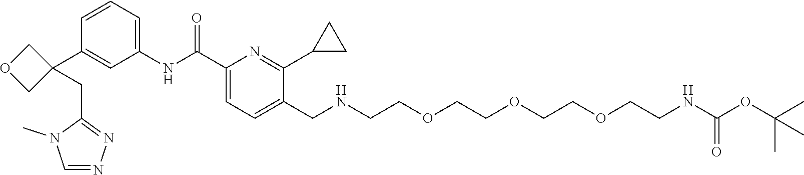 [SMILES: Cn1cnnc1CC1(c2cccc(NC(=O)c3ccc(CNCCOCCOCCOCCNC(=O)OC(C)(C)C)c(C4CC4)n3)c2)COC1]

A mixture of tert-butyl (2-{2-[2-(2-oxoethoxy)ethoxy]ethoxy}ethyl)carbamate (230 mg, 0.395 mmol), 5-(aminomethyl)-6-cyclopropyl-N-(3-{3-[(4-methyl-4H-1,2,4-triazol-3-yl)methyl]-oxetan-3-yl}phenyl)pyridine-2-carboxamide (165 mg, 0.395 mmol) and Ti(OEt)₄ (270 mg, 1.18 mmol) in THF (6 mL) was stirred at 20° C. for 16 h. Then NaBH(OAc)₃ (251 mg, 1.18 mmol) was added and stirred at 20° C. for 0.5 h. The reaction was quenched with sat. aq. Na₂CO₃ (5 mL), diluted with EtOAc (5 mL), filtered and the organic layer was separated, and the aqueous layer was extracted with EtOAc (5 mL). The combined organic layers were washed with brine (10 mL), dried over Na₂SO₄, filtered, concentrated in vacuo, and purified via preparative TLC (DCM:MeOH=10:1) to give tert-butyl (1-{2-cyclopropyl-6-[(3-{3-[(4-methyl-4H-1,2,4-triazol-3-yl)methyl]oxetan-3-yl}phenyl)carbamoyl]pyridin-3-yl}-5,8,11-trioxa-2-azatridecan-13-yl)carbamate as a yellow gum (80 mg, 29%).

### Step 7: 5-(13-amino-5,8,11-trioxa-2-azatridecan-1-yl)-6-cyclopropyl-N-(3-{3-[(4-methyl-4H-1,2,4-triazol-3-yl)methyl]oxetan-3-yl}phenyl)pyridine-2-carboxamide

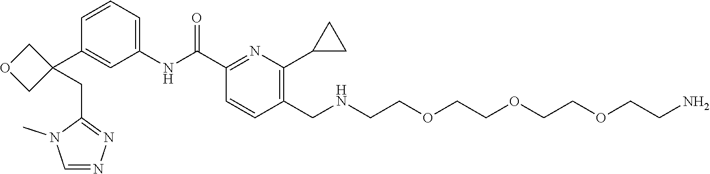 [SMILES: Cn1cnnc1CC1(c2cccc(NC(=O)c3ccc(CNCCOCCOCCOCCN)c(C4CC4)n3)c2)COC1]

To a solution of tert-butyl (1-{2-cyclopropyl-6-[(3-{3-[(4-methyl-4H-1,2,4-triazol-3-yl)methyl]oxetan-3-yl}phenyl)carbamoyl]pyridin-3-yl}-5,8,11-trioxa-2-azatridecan-13-yl)carbamate (80 mg, 0.0922 mmol) in DCM (2.5 mL) was added TFA (0.5 mL) and stirred at 25° C. for 40 min. The reaction solution was concentrated and basified to pH-8 with NH₃·H₂O. The residue was dissolved in MeOH (2 mL) and purified by preparative HPLC (Column: Phenemonex C18 column, 80×30 mm, 5 um column; Mobile phase A: Water+ammonia hydroxide Mobile phase B: Acetonitrile in 12 min, 35 mL/min) to give 5-(13-amino-5,8,11-trioxa-2-azatridecan-1-yl)-6-cyclopropyl-N-(3-{3-[(4-methyl-4H-1,2,4-triazol-3-yl)methyl]oxetan-3-yl}phenyl)pyridine-2-carboxamide as a yellow solid (32.9 mg, 57%). ¹H NMR (400 MHz, DMSO-d₆) δ 9.98 (s, 1H), 8.20 (s, 1H), 7.92-7.87 (m, 1H), 7.86-7.82 (m, 1H), 7.69 (br d, J=7.5 Hz, 1H), 7.40 (s, 1H), 7.27 (t, J=7.9 Hz, 1H), 6.65 (br d, J=7.8 Hz, 1H), 4.94 (d, J=6.0 Hz, 2H), 4.86 (d, J=6.0 Hz, 2H), 3.96 (s, 2H), 3.65-3.39 (m, 18H), 3.06 (br s, 1H), 2.92 (s, 3H), 2.73 (t, J=5.5 Hz, 2H), 2.42-2.34 (m, 1H), 1.24 (br d, J=2.0 Hz, 2H), 1.01 (br dd, J=2.9, 7.9 Hz, 2H). LCMS(ESI+) 594.5 (M+H)⁺.

### Synthesis of [6-cyclopropyl-5-(17-{5-[(3,5-dimethyl-2H-pyrrol-2-ylidene-κN)methyl]-1H-pyrrol-2-yl-κN}-15-oxo-5,8,11-trioxa-2,14-diazaheptadecan-1-yl)-N-(3-{3-[(4-methyl-4H-1,2,4-triazol-3-yl)methyl]oxetan-3-yl}phenyl)pyridine-2-carboxamidato](difluorido)boron

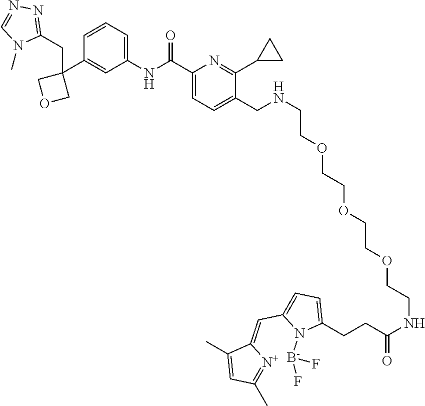

To a solution of BODIPY™ FL NHS ester (5 mg, 0.01 mmol, 1.00 equiv) in DCM (0.257 mL, 0.05 M) was added triethyl amine (8.95 uL, 0.0642 mmol, 5.00 equiv) and 5-(13-amino-5,8,11-trioxa-2-azatridecan-1-yl)-6-cyclopropyl-N-(3-{3-[(4-methyl-4H-1,2,4-triazol-3-yl)methyl]oxetan-3-yl}phenyl)pyridine-2-carboxamide (7.6 mg, 0.0161 mmol, 1.20 equiv) in DCM (0.3 mL). The reaction was covered with foil and stirred at room temperature. After 1.5 h, the reaction was stopped and purified via preparative HPLC (Phenemonex Gemini NX C18 column, 150×21.2 mm, 5 um; A: water+10 mM ammonium acetate, B: acetonitrile; 40 mL/min flow) to afford [6-cyclopropyl-5-(17-{5-[(3,5-dimethyl-2H-pyrrol-2-ylidene-κN)methyl]-1H-pyrrol-2-yl-κN}-15-oxo-5,8,11-trioxa-2,14-diazaheptadecan-1-yl)-N-(3-{3-[(4-methyl-4H-1,2,4-triazol-3-yl)methyl]oxetan-3-yl}phenyl)pyridine-2-carboxamidato](difluorido)boron (10.1 mg, 90%) as a red powder. LCMS (APCI+) 868.2 (M+H)⁺. 1H NMR (400 MHz, DMSO-d₆) δ 9.99-9.95 (m, 1H), 8.21-8.17 (m, 1H), 7.93-7.87 (m, 1H), 7.87-7.82 (m, 1H), 7.71-7.66 (m, 2H), 7.43-7.39 (m, 1H), 7.31-7.23 (m, 1H), 7.10-7.05 (m, 1H), 6.68-6.63 (m, 1H), 6.37-6.33 (m, 1H), 6.31-6.27 (m, 1H), 4.97-4.93 (m, 2H), 4.89-4.84 (m, 2H), 3.99-3.93 (m, 2H), 3.53-3.48 (m, 12H), 3.44-3.40 (m, 2H), 3.26-3.19 (m, 3H), 3.11-3.04 (m, 2H), 2.95-2.92 (m, 3H), 2.76-2.71 (m, 2H), 2.69-2.65 (m, 1H), 2.48-2.46 (m, 3H), 2.41-2.33 (m, 2H), 2.28-2.24 (m, 3H), 1.27-1.20 (m, 3H), 1.05-0.97 (m, 2H).

### Surface Plasmon Resonance (SPR) Assay

The purpose of the SPR assay is to evaluate the binding affinity and kinetics of the compound of disclosure binding to CBL-B and C-CBL.

The binding affinity and kinetics of compound binding to CBL-B and C-CBL were measured by Surface Plasmon Resonance (SPR) using either Biacore S200, 8K or 8K+ (Cytiva, Marlborough, MA) instruments. N-terminal biotinylated WT CBL-B (36-427) and C-CBL (46-435) were captured on a streptavidin (SA) coated sensor chip to achieve approximately 5000 to 6000 RUs of surface density. Un-functionalized SA surfaces with no immobilized protein served as reference for binding kinetic analysis. All the samples were prepared in buffer consisting of 10 mM HEPES, 150 mM NaCl, 0.05% Tween-20, 5% glycerol, 0.5 M TCEP, 2% DMSO, pH 7.2. The same buffer was used as the running buffer during the experiments. Compound binding kinetics were measured in either multi-cycle or single-cycle kinetic format.

Multi-cycle kinetic analysis (MCK): A two-fold, 10-point serial dilution of test compounds was set-up in a 96-well microplate (Greiner; Cat #650101) with a top concentration of either 10 μM or 100 μM. Binding kinetics was measured at 10° C. by injecting serial dilution of compounds onto both reference and CBL immobilized channels at a flow rate of 100 μL/min and association time of 90 seconds. Compound dissociation was monitored for >400 seconds during each cycle. No additional regeneration was used. DMSO calibration curve was obtained before and after compound analysis by injecting 0-4% of DMSO in assay buffer. A suitable compound with known affinity and kinetics was tested once in every experiment as a positive control to assess activity of the captured proteins on the surface.

Single-cycle kinetic analysis (SCK): A 3-fold, 6-point serial dilution of compounds was set-up in a deep 96-well microplate (Greiner Bio; Cat #780201) with the highest concentration of 1 μM (concentration range: 0.001-1 μM). Binding kinetics was measured at 10° C. by injecting serial dilutions of compounds in increasing order onto reference as well as CBL immobilized channels at a flow rate of 100 μL/min and association time of 120 seconds. Compound dissociation was monitored for >5400 seconds. Two buffer blanks were also run in a single-cycle kinetics format before the compound run for double referencing. No additional regeneration was used. DMSO calibration curve was obtained before and after compound analysis by injecting 0-4% of DMSO in the assay buffer. A suitable compound with known affinity and kinetics was tested once in every experiment as a positive control to assess activity of the captured proteins on the surface. SPR Assay binding data is provided in Table 15.

TABLE 15
SPR binding assay results

| | CBL-B SPR | | C-CBL SPR | |
|---|---|---|---|---|
| | Kd (nM) | Number of | Kd (nM) | Number of |
| EXAMPLE NO. | GMean | Tests | GMean | Tests |
| A1 | 34.6 | 3 | 930.9 | 3 |
| A2 | 35.8 | 1 | 251.0 | 1 |
| A3 | 12.9 | 1 | 301.0 | 1 |
| A6 | 21.4 | 1 | 882.0 | 1 |
| A7 | 31.0 | 1 | 1300.0 | 1 |
| B1 | 49.5 | 6 | 449.7 | 4 |
| B3 | 67.9 | 3 | 861.0 | 1 |
| B4 | 58.7 | 1 | 578.0 | 1 |
| B7 | 7.2 | 1 | 108.0 | 1 |
| B8 | 15.9 | 1 | 180.0 | 1 |
| B9 | 32.5 | 1 | 244.0 | 1 |
| B10 | 22.2 | 1 | 141.0 | 1 |
| C1 | 1.2 | 1 | 7.6 | 1 |
| C2 | 2.5 | 3 | 38.8 | 3 |
| C3 | 5.6 | 9 | 80.7 | 9 |
| C4 | 2.1 | 1 | 49.9 | 1 |
| C5 | 3.4 | 1 | 203.0 | 1 |
| C6 | 4.4 | 1 | 102.0 | 1 |
| C7 | 2.7 | 1 | 47.7 | 1 |
| C8 | 2.2 | 3 | 5.0 | 2 |
| C9 | 3.1 | 3 | 22.1 | 3 |
| C10 | 11.5 | 1 | 189.0 | 1 |
| C11 | 0.9 | 1 | 6.1 | 1 |
| C12 | 0.8 | 1 | 1.8 | 1 |
| C13 | 2.8 | 1 | 11.9 | 1 |
| C14 | 1.4 | 1 | 4.1 | 1 |
| C15 | 5.6 | 1 | 33.9 | 1 |
| C16 | 2.0 | 3 | 83.4 | 3 |
| C17 | 1.8 | 2 | 28.3 | 2 |
| C18 | 0.7 | 2 | 6.2 | 2 |
| C19 | 2.4 | 1 | 51.3 | 1 |
| C20 | 1.2 | 1 | 11.3 | 1 |
| C21 | 13.7 | 1 | 108.0 | 1 |
| C22 | 8.2 | 1 | 116.0 | 1 |
| C23 | 5.1 | 1 | 29.4 | 1 |
| C24 | 16.9 | 1 | 146.0 | 1 |
| C25 | 1.8 | 1 | 31.8 | 1 |
| C26 | 13.2 | 1 | 183.0 | 1 |
| C27 | 8.7 | 1 | 71.0 | 1 |
| C28 | 17.3 | 1 | 125.0 | 1 |
| C29 | 3.8 | 1 | 195.0 | 1 |
| C30 | 3.0 | 2 | 35.9 | 2 |
| C31 | 4.7 | 1 | 133.0 | 1 |
| C32 | 0.8 | 2 | 42.6 | 2 |
| C33 | 6.8 | 1 | 107.0 | 1 |
| C34 | 5.2 | 1 | 172.0 | 1 |
| C35 | 2.8 | 1 | 54.0 | 1 |
| C36 | 2.0 | 1 | 17.9 | 1 |
| C37 | 3.3 | 1 | 75.5 | 1 |
| C38 | 7.5 | 3 | 70.5 | 1 |
| C47 | 1.6 | 4 | 7.2 | 4 |
| C50 | 14.5 | 2 | 145.3 | 2 |
| C51 | 17.8 | 3 | 182.5 | 3 |
| C52 | 9.3 | 3 | 124.2 | 3 |
| C53 | 61.8 | 1 | 747.0 | 1 |
| C54 | 4.5 | 2 | 93.2 | 2 |
| C55 | 3.0 | 1 | 27.6 | 1 |
| C56 | 11.0 | 1 | 150.0 | 1 |
| C57 | 3.3 | 2 | 116.4 | 2 |
| C58 | 5.8 | 2 | 54.1 | 2 |
| C59 | 16.8 | 1 | 230.0 | 1 |
| C60 | 5.1 | 2 | 41.7 | 2 |
| C61 | 12.8 | 1 | 99.8 | 1 |
| C62 | 8.6 | 1 | 111.0 | 1 |
| C63 | 8.0 | 1 | 91.8 | 1 |
| C64 | 3.0 | 1 | 65.2 | 1 |
| C65 | 4.9 | 1 | 127.0 | 1 |
| C66 | 9.6 | 1 | 97.0 | 1 |
| C67 | 4.4 | 1 | 51.0 | 1 |
| C68 | 10.7 | 1 | 122.0 | 1 |
| C69 | 30.2 | 1 | 208.0 | 1 |
| C70 | 6.0 | 1 | 80.0 | 1 |
| C71 | 10.8 | 1 | 108.0 | 1 |
| C72 | 2.3 | 1 | 63.1 | 1 |
| C73 | 9.0 | 2 | 94.0 | 2 |
| C74 | 3.2 | 1 | 103.0 | 1 |
| C75 | 4.7 | 1 | 97.9 | 1 |
| C76 | 2.5 | 1 | 52.1 | 1 |
| C77 | 2.5 | 1 | 54.9 | 1 |
| C78 | 10.0 | 1 | 144.0 | 1 |
| C79 | 3.2 | 2 | 121.5 | 2 |
| C80 | 1.7 | 2 | 68.5 | 2 |
| C81 | 4.6 | 1 | 56.5 | 1 |
| C82 | 2.3 | 2 | 17.0 | 2 |
| C83 | 12.4 | 1 | 161.0 | 1 |
| C84 | 5.8 | 1 | 99.0 | 1 |
| C85 | 2.1 | 1 | 82.5 | 1 |
| C86 | 5.1 | 2 | 60.3 | 2 |
| C87 | 8.4 | 1 | 175.0 | 1 |
| C88 | 4.8 | 1 | 59.7 | 1 |
| C89 | 4.1 | 1 | 125.0 | 1 |
| C90 | 5.1 | 1 | 169.0 | 1 |
| C91 | 5.1 | 1 | 129.0 | 1 |
| C92 | 2.7 | 1 | 39.5 | 1 |
| C93 | 2.3 | 1 | 21.7 | 1 |
| C94 | 2.5 | 1 | 15.0 | 1 |
| C95 | 2.3 | 1 | 72.5 | 1 |
| C96 | 3.3 | 1 | 91.2 | 1 |
| C97 | 9.6 | 1 | 242.0 | 1 |
| C98 | 11.3 | 1 | 205.0 | 1 |
| C103 | 20.5 | 1 | 349.0 | 1 |
| C104 | 8.2 | 1 | 328.0 | 1 |
| C105 | 4.2 | 1 | 132.0 | 1 |
| C106 | 5.9 | 1 | 265.0 | 1 |
| C107 | 3.7 | 1 | 158.0 | 1 |
| C108 | 5.5 | 1 | 148.0 | 1 |
| C109 | 8.8 | 1 | 174.0 | 1 |
| C110 | 6.4 | 1 | 300.0 | 1 |
| C111 | 3.3 | 1 | 282.0 | 1 |
| C112 | 3.0 | 1 | 82.1 | 1 |
| C113 | 4.8 | 1 | 83.6 | 1 |
| C114 | 10.8 | 1 | 278.0 | 1 |
| C115 | 9.2 | 1 | 195.0 | 1 |
| C119 | 21.9 | 1 | 254.0 | 1 |
| C120 | 11.1 | 1 | 156.0 | 1 |
| C121 | 3.3 | 1 | 113.0 | 1 |
| C122 | 5.5 | 1 | 146.0 | 1 |
| D1 | 3.7 | 3 | 207.5 | 3 |
| D2 | 3.8 | 1 | 108.0 | 1 |
| D3 | 4.0 | 1 | 124.0 | 1 |
| D4 | 2.8 | 3 | 106.1 | 3 |
| D5 | 21.9 | 1 | 209.0 | 1 |
| D6 | 1.8 | 1 | 31.0 | 1 |
| D7 | 2.3 | 1 | 36.0 | 1 |
| D8 | 15.8 | 1 | 177.0 | 1 |
| D9 | 8.3 | 1 | 96.0 | 1 |
| D10 | 2.8 | 1 | 42.0 | 1 |
| D11 | 4.2 | 1 | 47.9 | 1 |
| D12 | 6.6 | 1 | 46.0 | 1 |
| D13 | 3.5 | 1 | 51.2 | 1 |
| D14 | 11.1 | 1 | 140.0 | 1 |
| D15 | 2.5 | 1 | 6.2 | 1 |
| D16 | 3.6 | 1 | 43.1 | 1 |
| D19 | 1.6 | 2 | 17.2 | 2 |
| D22 | 1.5 | 2 | 9.8 | 2 |
| D28 | 0.4 | 3 | 2.2 | 3 |
| D30 | 1.1 | 2 | 5.6 | 2 |
| D34 | 1.8 | 1 | 101.0 | 1 |
| D35 | 4.4 | 1 | 152.0 | 1 |
| D37 | 7.9 | 1 | 68.6 | 1 |
| D38 | 10.0 | 1 | 110.0 | 1 |
| D39 | 14.9 | 1 | 164.0 | 1 |
| D40 | 5.5 | 1 | 88.5 | 1 |
| D41 | 6.2 | 1 | 50.8 | 1 |
| D42 | 9.4 | 1 | 59.8 | 1 |
| D43 | 6.7 | 1 | 80.2 | 1 |
| D44 | 5.9 | 1 | 74.0 | 1 |
| D45 | 5.2 | 1 | 83.9 | 1 |
| D46 | 12.2 | 1 | 184.0 | 1 |
| D47 | 16.3 | 2 | 192.7 | 2 |
| D48 | 9.3 | 1 | 74.6 | 1 |
| D49 | 5.9 | 1 | 82.0 | 1 |
| D50 | 2.6 | 1 | 30.1 | 1 |
| D51 | 2.7 | 1 | 15.0 | 1 |
| D52 | 10.1 | 1 | 79.1 | 1 |
| D53 | 3.2 | 1 | 36.1 | 1 |
| D54 | 8.2 | 1 | 61.7 | 1 |
| D80 | 9.5 | 1 | 306.0 | 1 |
| D81 | 5.6 | 1 | 206.0 | 1 |
| D82 | 8.7 | 1 | 213.0 | 1 |
| D83 | 7.6 | 1 | 177.0 | 1 |
| D84 | 5.6 | 1 | 148.0 | 1 |
| D85 | 6.9 | 1 | 241.0 | 1 |
| D86 | 6.4 | 1 | 161.0 | 1 |
| D87 | 4.8 | 1 | 128.0 | 1 |
| D88 | 5.7 | 1 | 34.1 | 1 |
| D89 | 11.0 | 1 | 307.0 | 1 |
| D90 | 6.5 | 1 | 77.4 | 1 |
| D91 | 6.3 | 1 | 35.4 | 1 |
| D94 | 5.5 | 1 | 163.0 | 1 |
| D95 | 6.5 | 1 | 226.0 | 1 |
| D96 | 5.5 | 1 | 120.0 | 1 |
| D97 | 31.9 | 4 | 230.0 | 4 |
| D98 | 46.0 | 6 | 217.1 | 4 |
| E2 | 53.9 | 1 | 1730.0 | 1 |
| F1 | 0.7 | 3 | 11.2 | 3 |
| G1 | 8.0 | 3 | 60.8 | 3 |
| J1 | 4.3 | 1 | 36.7 | 1 |
| J2 | 4.8 | 1 | 37.6 | 1 |
| J3 | 3.2 | 1 | 53.6 | 1 |
| J4 | 3.7 | 1 | 145.0 | 1 |
| J5 | 3.3 | 1 | 137.0 | 1 |
| J8 | 16.1 | 1 | 136.0 | 1 |
| J9 | 30.1 | 1 | 244.0 | 1 |
| J10 | 21.4 | 2 | 178.3 | 2 |
| J11 | 24.0 | 1 | 393.0 | 1 |
| J12 | 16.1 | 1 | 240.0 | 1 |
| K3 | 4.0 | 1 | 160.0 | 1 |
| K4 | 8.3 | 1 | 207.0 | 1 |
| K5 | 23.6 | 2 | 221.0 | 1 |
| K6 | 11.3 | 2 | 125.3 | 2 |
| K7 | 8.0 | 2 | 94.9 | 2 |
| K9 | 12.7 | 1 | 298.0 | 1 |
| K10 | 23.5 | 1 | 291.0 | 1 |
| K11 | 11.7 | 1 | 199.0 | 1 |
| K12 | 4.2 | 1 | 209.0 | 1 |
| K13 | 12.1 | 1 | 285.0 | 1 |
| K14 | 20.6 | 1 | 244.0 | 1 |
| K15 | 23.9 | 1 | 452.0 | 1 |
| K16 | 15.7 | 3 | 216.0 | 1 |
| K17 | 27.3 | 3 | 256.0 | 1 |
| L1 | 40.7 | 1 | 697.0 | 1 |
| L2 | 30.0 | 1 | 307.0 | 1 |
| M4 | 10.3 | 1 | 250.0 | 1 |
| M5 | 14.2 | 1 | 741.0 | 1 |
| N1 | 35.8 | 1 | 275.0 | 1 |

Jurkat HiBit-IL2 Assay (WT, c-CBL KO, CBL-B KO)

The purpose of the Jurkat HiBit-IL2 assay is to evaluate the effect of the inhibition of CBL-B and/or C-CBL on TCR/CD28 stimulated interleukin-2 (IL-2) secretion.

The production of IL-2 by CD4+ T cells upon TCR/CD28 stimulation is significantly enhanced upon the inhibition of C-CBL and/or CBL-B. A GFP-T2A-FLAG-HiBit-IL2 Jurkat cell line (Jurkat HiBit-IL2 reporter) was generated by CRISPR such that the endogenous IL-2 gene was tagged with an in-frame GFP-T2A-FLAG-HiBit sequence at its N-terminus. The expression of IL-2 can then be accurately measured by HiBit luciferase assay. Further, C-CBL or CBL-B was knocked out by CRISPR respectively in this Jurkat HiBit-IL2 reporter cell line to create reporter cell line expressing CBL-B only (c-CBL knockout (KO)) or C-CBL only (CBL-B KO).

All three reporter cell lines were maintained in RPMI-1640 media with GlutaMax, 10% fetal bovine serum, 100 units/ml of penicillin, and 100 μg/ml of streptomycin at 37° C., 5% CO₂, at a cell density 1×10e5-2×10e6 viable cells/ml. Serial diluted compounds in DMSO (40 nl/well, final 0.1% DMSO in assay cell culture) were plated into 384-well white-wall clear-bottom cell culture plates. Reporter cells were pelleted and resuspended in fresh cell culture medium to 0.66×10e6 cells/ml and added to the assay plate at 30 μl/well (20,000 cells/well). The cells were incubated with compounds for 1 hour at 37° C., 5% CO₂. 10 μl/well of anti-CD3 antibody (OKT3) and anti-CD28 antibody (CD28.2) prediluted in fresh cell culture medium at 4× final concentration (anti-CD3 mAb: 4 μg/ml; anti-CD28 mAb: 8 μg/ml) was added into the cells (final concentration of anti-CD3 mAb: 1 μg/ml; anti-CD28 mAb: 2 μg/ml). The cells were cultured for another 4 hours at 37° C., 5% CO₂. The Nano-Glo HiBiT Lytic Reagent was prepared following the manufacturer's protocol. 20 ul/well of the Nano-Glo HiBiT Lytic Reagent was added into the reporter cell culture assay plate and incubated at room temperature (20-25° C.) for 15 minutes. The plates were read on a Perkin-Elmer EnVision plate reader with 0.1 second integration time. Data were normalized to “Fold of Effect” with the anti-CD3 and anti-CD28 antibodies only samples as the baseline activation level and EC50 was calculated. Jurkat HiBit-IL2 assay data is provided in Tables 16a and 16b. The EC₅₀ (nM) values reported are the geometric means (GMean) of the number of tests conducted.

TABLE 16a
Jurkat HiBit-IL2 assay results

| | C-CBL KO | | CBL-B KO | |
|---|---|---|---|---|
| EXAMPLE | EC₅₀ (nM) | Number | EC₅₀ (nM) | Number |
| NO. | GMean | of Tests | GMean | of Tests |
| A1 | >2990 | 4 | >5281 | 4 |
| B1 | >8711 | 2 | >9980 | 1 |
| B3 | 1710 | 2 | 4065 | 2 |
| C1 | 25 | 6 | 275 | 5 |
| C2 | 54 | 10 | 641 | 10 |
| C3 | >159 | 7 | 639 | 7 |
| C4 | 114 | 4 | 1046 | 4 |
| C5 | 159 | 2 | 1248 | 2 |
| C6 | 115 | 3 | 807 | 3 |
| C7 | 58 | 3 | 519 | 3 |
| C8 | 6 | 6 | 44 | 6 |
| C9 | 32 | 6 | 606 | 6 |
| C10 | 233 | 5 | >2313 | 5 |
| C11 | 5 | 5 | 46 | 5 |
| C12 | 4 | 3 | 25 | 3 |
| C13 | 16 | 4 | 63 | 4 |
| C14 | 4 | 3 | 26 | 3 |
| C15 | 43 | 4 | 229 | 4 |
| C16 | 75 | 4 | 689 | 4 |
| C17 | 33 | 5 | 196 | 5 |
| C18 | 17 | 5 | 178 | 5 |
| C19 | 56 | 3 | 439 | 3 |
| C20 | 12 | 3 | 78 | 3 |
| C21 | 152 | 3 | 851 | 3 |
| C22 | 120 | 3 | 942 | 3 |
| C23 | 152 | 2 | 800 | 2 |
| C24 | 434 | 1 | 2969 | 1 |
| C25 | 49 | 2 | 252 | 2 |
| C26 | 172 | 2 | 1852 | 2 |
| C27 | >1294 | 2 | >4302 | 2 |
| C28 | >2067 | 2 | >5194 | 2 |
| C29 | 204 | 4 | 1318 | 4 |
| C30 | 32 | 5 | 232 | 5 |
| C31 | 166 | 4 | 1372 | 4 |
| C32 | 83 | 4 | 1194 | 4 |
| C33 | 116 | 3 | 1843 | 3 |
| C34 | 67 | 3 | 892 | 3 |
| C35 | 15 | 5 | 172 | 4 |
| C36 | 20 | 9 | 173 | 8 |
| C37 | 173 | 4 | 1038 | 4 |
| C38 | 71 | 3 | 494 | 3 |
| C39 | 35 | 4 | 496 | 3 |
| C40 | 47 | 4 | 1359 | 3 |
| C41 | 42 | 4 | 327 | 3 |
| C42 | 29 | 4 | 316 | 3 |
| C43 | 51 | 5 | 912 | 4 |
| C44 | 21 | 5 | 253 | 4 |
| C45 | 14 | 5 | 102 | 4 |
| C46 | 36 | 4 | 392 | 3 |
| C47 | 5 | 9 | 105 | 8 |
| C48 | 20 | 4 | 493 | 4 |
| C49 | 8 | 4 | 135 | 4 |
| D1 | 276 | 11 | 1894 | 11 |
| D2 | 42 | 12 | 465 | 11 |
| D3 | 47 | 3 | 472 | 3 |
| D4 | 72 | 6 | 479 | 6 |
| D5 | 650 | 2 | 1376 | 2 |
| D6 | 64 | 3 | 235 | 3 |
| D7 | 80 | 3 | 380 | 3 |
| D8 | 603 | 2 | 1211 | 2 |
| D9 | 627 | 2 | 1765 | 2 |
| D10 | 102 | 3 | 595 | 3 |
| D11 | 143 | 3 | 512 | 3 |
| D12 | 612 | 2 | 973 | 2 |
| D13 | 149 | 3 | 539 | 3 |
| D14 | 354 | 2 | 2010 | 2 |
| D15 | 3 | 11 | 20 | 10 |
| D16 | 18 | 11 | 188 | 10 |
| D17 | 19 | 6 | 270 | 5 |
| D18 | 10 | 5 | 96 | 4 |
| D19 | 9 | 11 | 132 | 10 |
| D20 | 4 | 5 | 30 | 4 |
| D21 | 39 | 5 | 420 | 4 |
| D22 | 5 | 8 | 79 | 7 |
| D23 | 6 | 5 | 63 | 4 |
| D24 | 22 | 7 | 285 | 6 |
| D25 | 20 | 4 | 174 | 3 |
| D26 | 9 | 4 | 122 | 3 |
| D27 | 11 | 8 | 194 | 7 |
| D28 | 1 | 7 | 4 | 6 |
| D29 | 11 | 4 | 139 | 3 |
| D30 | 3 | 4 | 43 | 3 |
| D31 | 16 | 4 | 199 | 3 |
| D32 | 8 | 4 | 175 | 4 |
| F1 | 8 | 20 | 60 | 19 |
| G1 | 70 | 6 | 775 | 6 |
| H1 | 44 | 4 | 559 | 4 |
| H2 | 17 | 4 | 232 | 4 |
| H3 | 49 | 4 | 392 | 4 |
| H4 | 5 | 4 | 43 | 4 |
| I1 | 16 | 3 | 182 | 3 |
| I2 | 313 | 3 | 2417 | 3 |
| I3 | 3 | 3 | 17 | 3 |
| I4 | 48 | 3 | 852 | 3 |

Further repetition of the Jurkat HiBit-IL2 assay provided the results below in Table 16b.

TABLE 16b
Jurkat HiBit-IL2 assay results

| | C-CBL KO | | CBL-B KO | |
|---|---|---|---|---|
| EXAMPLE | EC₅₀ (nM) | Number | EC₅₀ (nM) | Number |
| NO. | GMean | of Tests | GMean | of Tests |
| A1 | 1815 | 1 | 4497 | 1 |
| C1 | 116 | 1 | 584 | 1 |
| C9 | 32 | 2 | 176 | 2 |
| C11 | 6 | 3 | 32 | 3 |
| C12 | 8 | 2 | 16 | 2 |
| C14 | 5 | 3 | 17 | 3 |
| C18 | 19 | 1 | 49 | 1 |
| C20 | 15 | 2 | 56 | 2 |
| C32 | 49 | 1 | 110 | 1 |
| C34 | 86 | 1 | 428 | 1 |
| C36 | 35 | 23 | 160 | 23 |
| C39 | 108 | 3 | 615 | 3 |
| C40 | 214 | 2 | 1560 | 2 |
| C41 | 154 | 1 | 877 | 1 |
| C42 | 94 | 3 | 635 | 3 |
| C43 | 61 | 1 | 554 | 1 |
| C44 | 60 | 2 | 356 | 2 |
| C45 | 30 | 1 | 115 | 1 |
| C46 | 61 | 3 | 330 | 3 |
| C47 | 11 | 26 | 76 | 26 |
| C48 | 67 | 3 | 466 | 3 |
| C89 | 85 | 1 | 471 | 1 |
| C92 | 113 | 1 | 525 | 1 |
| C95 | 53 | 1 | 230 | 1 |
| C96 | 76 | 1 | 574 | 1 |
| C99 | 44 | 3 | 323 | 3 |
| C100 | 110 | 3 | 1415 | 3 |
| C101 | 58 | 5 | 909 | 5 |
| C117 | 170 | 1 | 794 | 1 |
| C118 | 46 | 5 | 338 | 5 |
| C123 | 185 | 1 | 1071 | 1 |
| C124 | 348 | 1 | 3282 | 1 |
| D2 | 83 | 1 | 425 | 1 |
| D3 | 67 | 1 | 333 | 1 |
| D15 | 4 | 8 | 11 | 8 |
| D17 | 43 | 3 | 552 | 3 |
| D18 | 22 | 2 | 137 | 2 |
| D19 | 24 | 21 | 157 | 21 |
| D20 | 11 | 1 | 31 | 1 |
| D21 | 107 | 2 | 1148 | 2 |
| D22 | 12 | 4 | 91 | 4 |
| D23 | 21 | 2 | 124 | 2 |
| D24 | 58 | 2 | 452 | 2 |
| D25 | 101 | 2 | 359 | 2 |
| D26 | 30 | 3 | 180 | 3 |
| D27 | 24 | 3 | 209 | 3 |
| D29 | 36 | 3 | 224 | 3 |
| D30 | 10 | 3 | 52 | 3 |
| D31 | 56 | 3 | 258 | 3 |
| D32 | 12 | 3 | 134 | 3 |
| D36 | 52 | 2 | 454 | 2 |
| D55 | 80 | 3 | 642 | 3 |
| D56 | 84 | 1 | 365 | 1 |
| D59 | 267 | 2 | 1259 | 2 |
| D63 | 121 | 3 | 577 | 3 |
| D64 | 134 | 1 | 483 | 1 |
| D65 | 468 | 1 | 1182 | 1 |
| D66 | 72 | 2 | 409 | 2 |
| D67 | 321 | 2 | 1520 | 2 |
| D68 | 293 | 2 | 1708 | 2 |
| D69 | 154 | 2 | 490 | 2 |
| D70 | 163 | 2 | 618 | 2 |
| D71 | 77 | 3 | 434 | 3 |
| D72 | 103 | 1 | 563 | 1 |
| D73 | 118 | 1 | 639 | 1 |
| D74 | 639 | 1 | 3401 | 1 |
| D75 | 195 | 1 | 1027 | 1 |
| D76 | 20 | 1 | 39 | 1 |
| D77 | 33 | 2 | 93 | 2 |
| D90 | 73 | 3 | 313 | 3 |
| D91 | 41 | 3 | 238 | 3 |
| F1 | 15 | 23 | 56 | 22 |
| H1 | 103 | 2 | 485 | 2 |
| H2 | 41 | 2 | 165 | 2 |
| H3 | 231 | 1 | 415 | 1 |
| H4 | 11 | 2 | 28 | 2 |
| I1 | 29 | 3 | 163 | 3 |
| I3 | 6 | 5 | 15 | 5 |
| I4 | 108 | 2 | 491 | 2 |
| I5 | 62 | 3 | 299 | 3 |
| K12 | 895 | 3 | 2084 | 3 |

The data shown above confirms that inhibition of CBL-B affords functional activity in cellular systems.

### Human PBMC Assay

The purpose of the human PBMC assay is to evaluate the antigen-dependent and antigen-independent CD8 T-cell activation.

Human peripheral blood mononuclear cells (PBMCs) consist of T cells, B cells, NK cells, macrophages, and monocytes. Specific antigen peptides presented by MHC I-expressing PBMC cells can be recognized by cognate T cell receptors (TCR) on CD8 T cells and lead to their activation. CEF viral peptides (CEF-MHC Class I Control Peptide Pool “Plus”, Immunospot, Catalog #CTL-CEF-002) contain 32 well-defined MHC I-restricted viral epitopes from Cytomegalovirus, Epstein-Barr virus, and Influenza viruses, which most human individuals have been previously exposed to and generated effector/memory CD8 T cells specific to the viruses. Therefore, the majority of randomly selected human donors will respond to the CEF viral peptides challenge and reactivate the viral antigens-specific CD8 T cells. The production of IFNg is a major hallmark of CD8 T cell activation and was used to measure the level of CD8 T cells activation quantitatively upon antigen challenges.

To properly evaluate the effect of test compounds in antigen-dependent and -independent CD8 T cells activation, serial diluted compounds in DMSO (100 nl/well, final 0.1% DMSO in assay cell culture) were plated into 96-well cell culture plates using a Tecan D300e Digital Dispenser. The cryopreserved whole human PBMCs isolated from human blood leukopak (StemCell, Catalog #70500, no gender preference, donors younger than 45, no health issues) by Ficoll or similar method were washed and resuspended to 3.33×10e6 cells/ml in fresh X-VIVO™ 15 Serum-free Hematopoietic Cell Medium (Lonza, Catalog #04-418Q) and added to the assay plate at 75 μl/well (0.25×10e6 cells/well). The cells were incubated with compounds for 1 hour at 37° C., 5% CO₂ followed by the adding of 25 μl/well of CEF viral peptides prediluted in fresh cell culture medium at 4× final concentration (0.4 μg/ml for each peptide, final concentration 0.1 μg/ml), or 25 μl/well of fresh cell culture medium only. The cells were then cultured at 37° C., 5% CO₂ and measured for IFNg production with V-PLEX Human IFN-γ Kit (MSD, Catalog #K151QOD) after 3 days. The level of IFN-γ production was plotted against the compound concentrations to depict the dosage response of CD8 T cell activation upon compound treatment in the presence or absence of specific antigen peptides.

It will be apparent to those skilled in the art that various modifications and variations may be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula (I): 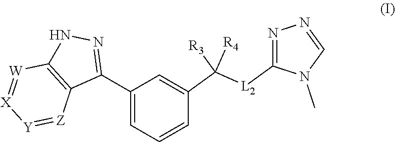
or a pharmaceutically acceptable salt thereof,
wherein:
W is N or C-L₁-R₁;
X is N or C-L₁-R₁;
Y is N or CR₂;
Z is N or CH;
with the provisos that:
one of W and X is N or CH and the other one of W and X is C-L₁-R₁;
when Z is N, X is N; and
Y and Z are not both N;
R₁ is selected from the group consisting of H, halogen, C₃-C₁₀ cycloalkyl, 4-8 membered heterocycloalkyl, NR₅R₆, and OR₇; wherein said C₃-C₁₀ cycloalkyl or said 4-8 membered heterocycloalkyl ring is optionally substituted by one or more R₉ which can be the same or different;
R₂ is selected from the group consisting of H, halogen, C₁-C₆ alkyl, C₁-C₆ haloalkyl, C₁-C₆ alkoxy and C₃-C₁₀ cycloalkyl; wherein said C₁-C₆ alkyl, said C₁-C₆ alkoxy or said C₃-C₁₀ cycloalkyl is optionally substituted by one or more halogen or oxo;
R₃ and R₄ are independently selected from the group consisting of H, fluoro, C₁-C₆ alkyl, C₃-C₁₀ cycloalkyl and 4-8 membered heterocycloalkyl, each of which is optionally substituted by one or more R₁₀ which can be the same or different; or R₃ and R₄ together with the carbon atom to which they are attached form a C₃-C₈ cycloalkyl or 4-8 membered heterocycloalkyl ring, each of which is optionally substituted by one or more R₁₀ which can be the same or different;
R₅ and R₆ are independently H, C(O)R₈, C(O)OR₈, C(O)NHR₈, SOR₈, SO₂R₈, C₁-C₆ alkyl, C₃-C₁₀ cycloalkyl, 4-8 membered heterocycloalkyl, wherein said C₁-C₆ alkyl, C₃-C₁₀ cycloalkyl or said 4-8 membered heterocycloalkyl is optionally substituted by one or more R₉ which can be the same or different; or R₅ and R₆ together with the nitrogen atom to which they are attached form a 4-8 membered heterocycloalkyl ring optionally further containing one or more heteroatoms selected from oxygen, sulfur, and nitrogen, wherein said 4-8 membered heterocycloalkyl ring is optionally substituted by one or more R₉ which can be the same or different;
R₇ is H, C₁-C₆ alkyl or 4-8 membered heterocycloalkyl, wherein said C₁-C₆ alkyl or said C₃-C₁₀ heterocycloalkyl is optionally substituted by one or more R₉ which can be the same or different;
R₈ is C₁-C₆ alkyl optionally substituted by one or more R₉ which can be the same or different;
R₉ is each independently halogen, C₁-C₆ alkyl, OH, hydroxyalkyl, C₁-C₃ alkoxy, NR₁₁R₁₂, NR₁₁COR₁₂, NR₁₁SO₂R₁₂, NR₁₁COOR₁₂, NR₁₁CONR₁₂R₁₃, SR₁₁, SOR₁₁, SO₂ R₁₁, CONR₁₁R₁₂, CN or oxo;
R₁₀ is each independently halogen, C₁-C₃ alkyl, C₁-C₃ alkoxy, fluoroalkyl, CN, oxo or OH;
R₁₁, R₁₂ and R₁₃ are each independently H or C₁-C₃ alkyl;
L₁ and L₂ are independently a bond or a C₁-C₃ alkylene optionally substituted by one or more F, C₁-C₃ alkyl, C₁-C₃ alkoxy, fluoroalkyl or OH, and
wherein 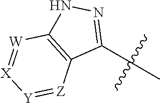
is selected from the group consisting of:

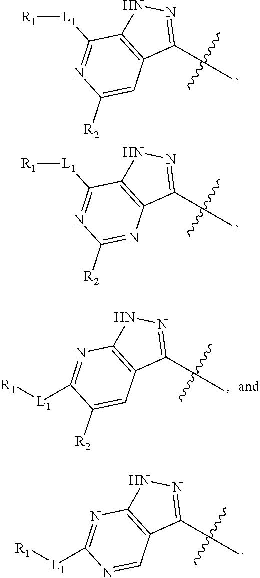

2. (canceled)

3. The compound or pharmaceutically acceptable salt of claim 1, having the Formula (II):

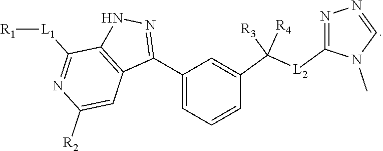

4. The compound or pharmaceutically acceptable salt of claim 1, having the Formula (II-a), (II-b), (II-c), (II-d), (II-e), or (II-f):

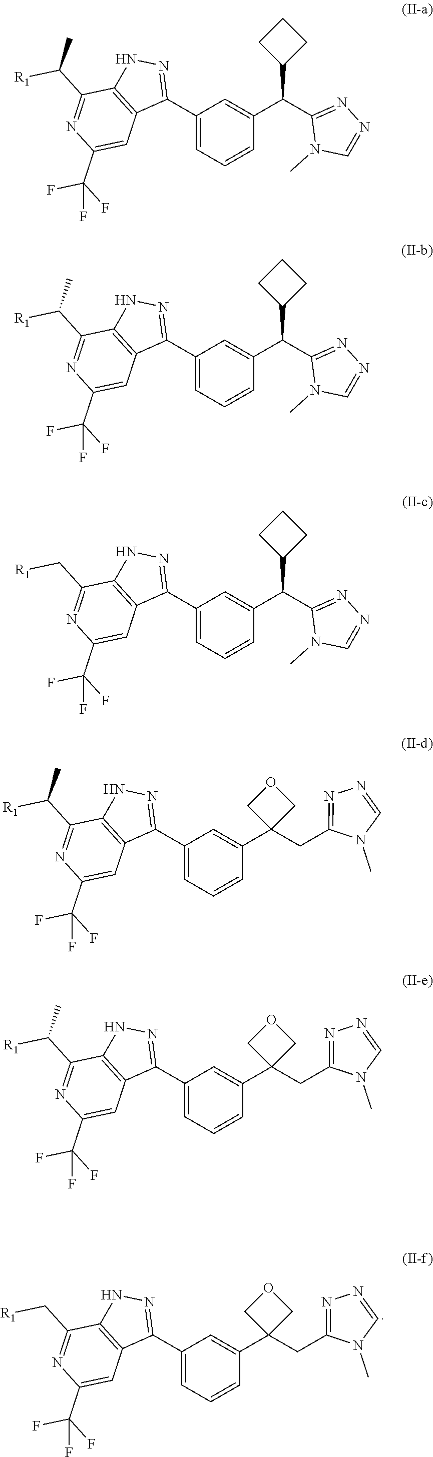

5. (canceled)

6. The compound or pharmaceutically acceptable salt of claim 1, wherein R₁ is NR₅R₆, further wherein one of R₅ and R₆ is H and the other one of R₅ and R₆ is C₁-C₆ alkyl, C₃-C₁₀ cycloalkyl, 4-8 membered heterocycloalkyl, C(O)R₈, C(O)OR₈, C(O)NHR₈, SOR₈, or SO₂R₈, further wherein said C₁-C₆ alkyl, said C₃-C₁₀ cycloalkyl, or said 4-8 membered heterocycloalkyl is optionally substituted by one or more R₉ which can be the same or different.

7. The compound or pharmaceutically acceptable salt of claim 6, wherein one of R₅ and R₆ is selected from the group consisting of C₁-C₃ alkyl, cyclopropyl, cyclobutyl, cyclopentyl, tetrahydrofuranyl, and azetidinyl, where each of which is optionally substituted by one or more R₉ which can be the same or different, or R₅ and R₆ together with the nitrogen atom to which they are attached form a 4-8 membered heterocycloalkyl ring selected from the group consisting of: 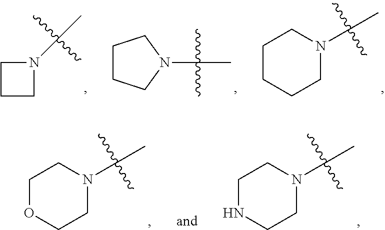 [SMILES: *N1CCC1 ; *N1CCCC1 ; *N1CCCCC1 ; *N1CCOCC1 ; *N1CCNCC1]
optionally substituted by one or more R₉ which can be the same or different.

8. (canceled)

9. (canceled)

10. The compound or pharmaceutically acceptable salt of claim 1, wherein each R₉ is independently halogen, OH, NH₂, C₁-C₆ alkyl, or oxo.

11. The compound or pharmaceutically acceptable salt of claim 1, wherein R₂ is CF₃.

12. (canceled)

13. The compound or pharmaceutically acceptable salt of claim 1, wherein R₃ is cyclobutyl.

14. The compound or pharmaceutically acceptable salt of claim 1, wherein R₃ and R₄ together with the carbon atom to which they are attached to form a 4-8 membered heterocycloalkyl ring.

15. The compound or pharmaceutically acceptable salt of claim 14, wherein R₃ and R₄ together with the carbon atom to which they are attached to form an optionally substituted oxetane.

16. (canceled)

17. The compound or pharmaceutically acceptable salt of claim 1, wherein L₁ is selected from —CH₂— or —CHCH₃—.

18. The compound or pharmaceutically acceptable salt of claim 1, wherein L₂ is selected from a bond, —CH₂— or —CHCH₃—.

19. The compound or pharmaceutically acceptable salt of claim 1, wherein L₂ is a bond; R₃ is cyclobutyl; and R₄ is H.

20. The compound or pharmaceutically acceptable salt of claim 1, wherein L₂ is CH₂; and R₃ and R₄ together with the carbon atom to which they are attached to form an oxetane.

21. The compound of claim 1, or a pharmaceutically acceptable salt thereof, wherein the compound is selected from the group consisting of:

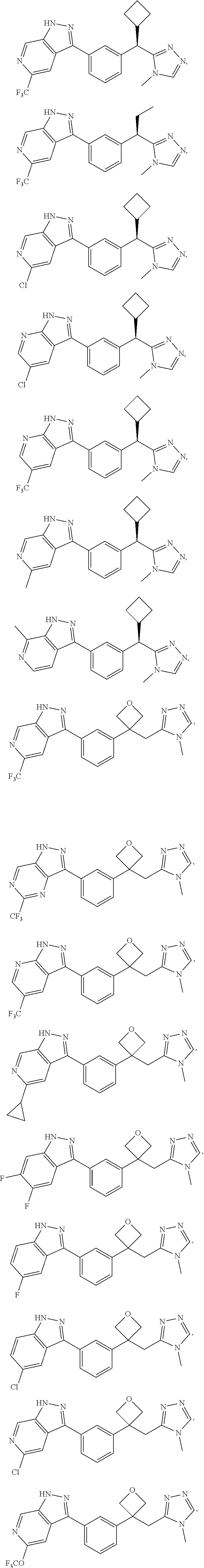

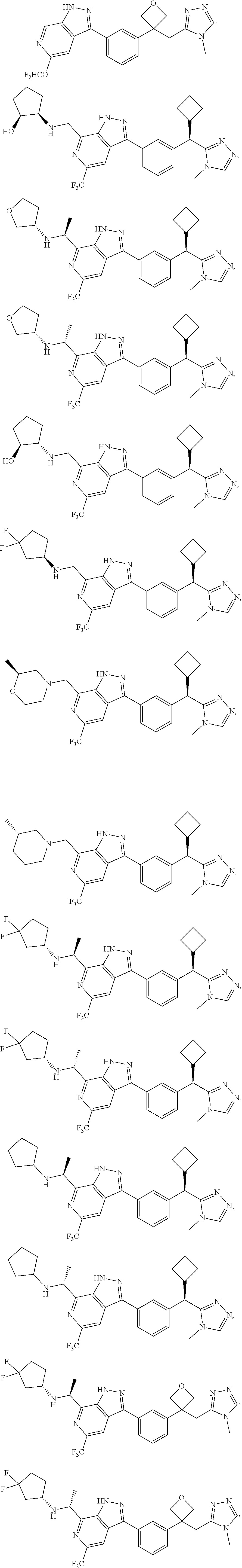

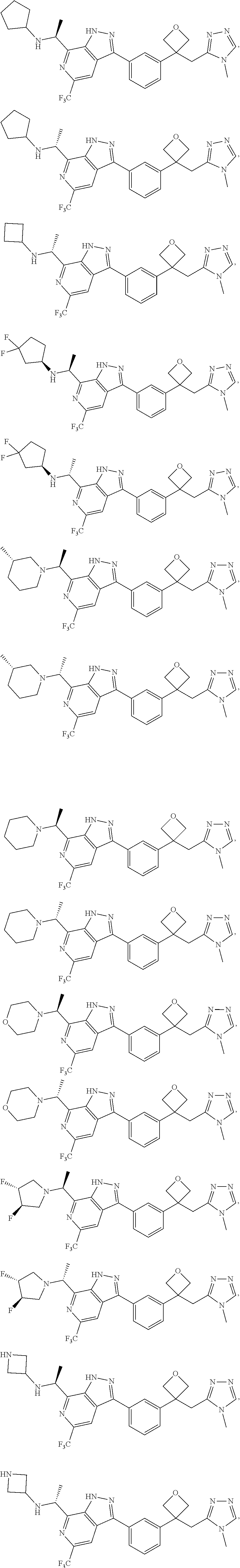

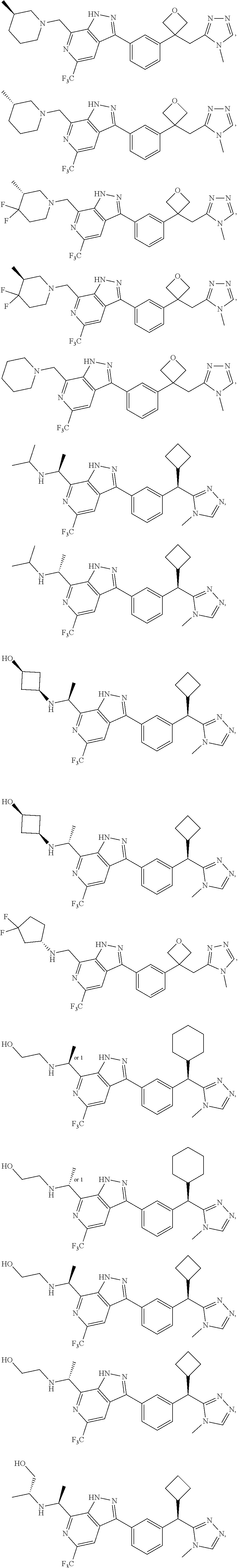

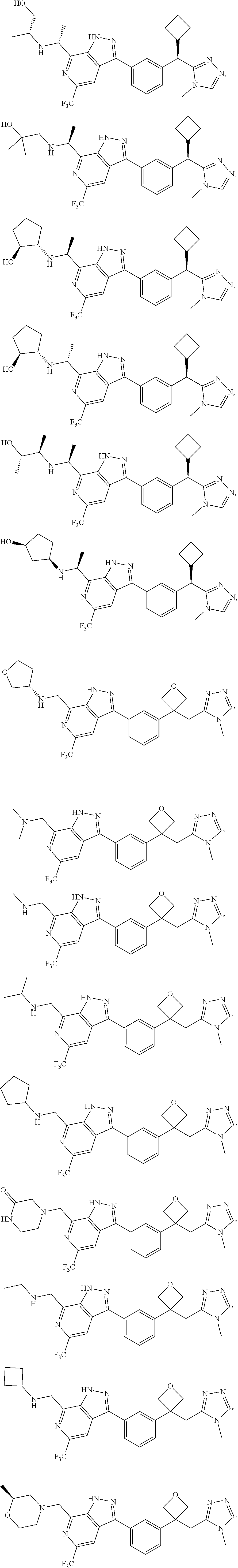

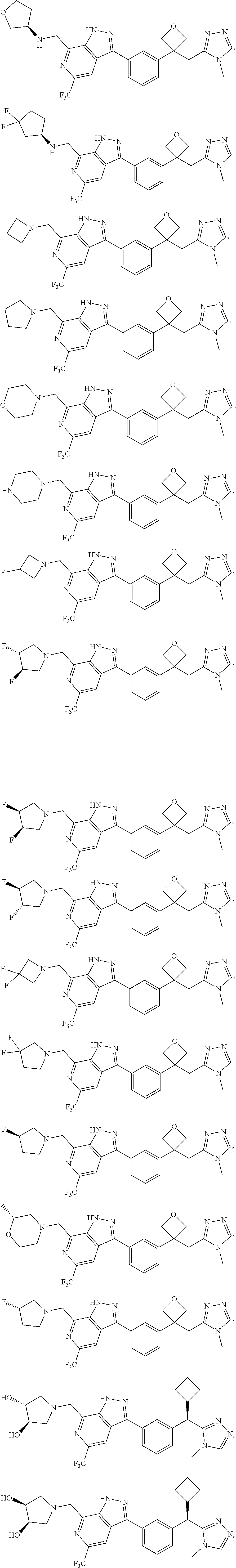

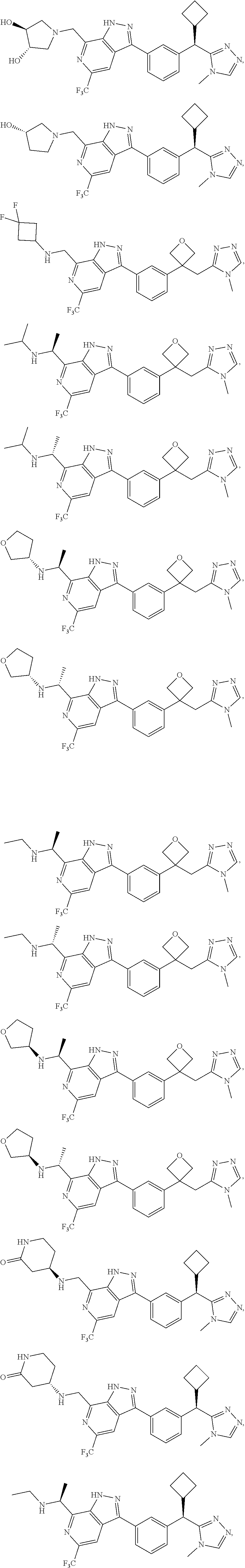

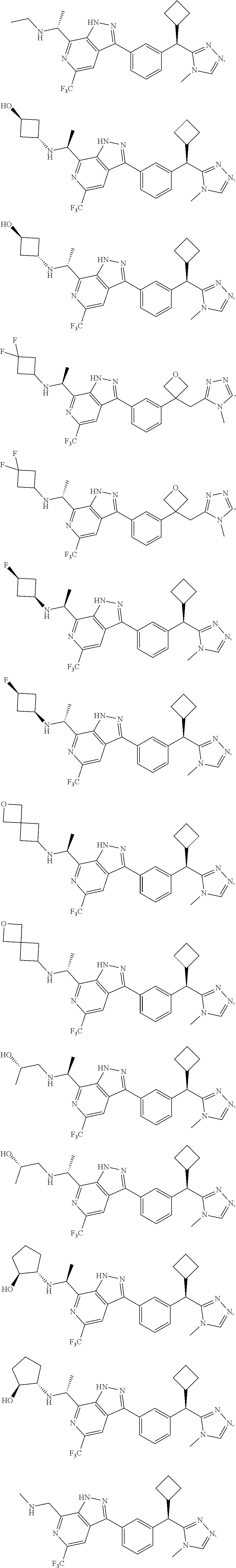

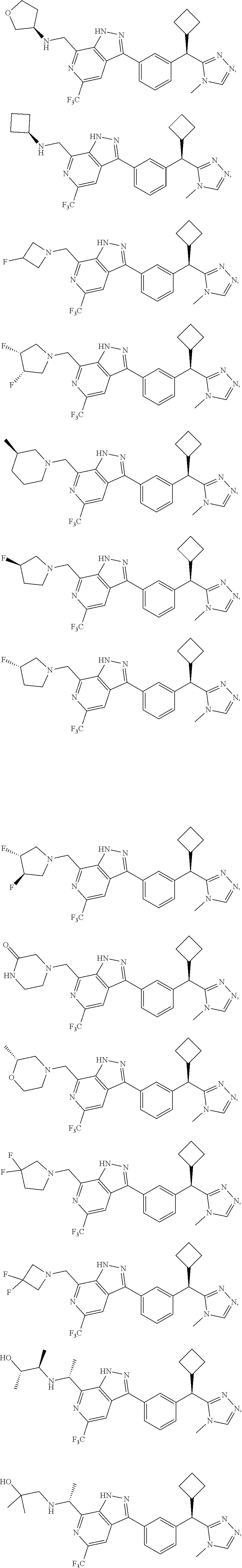

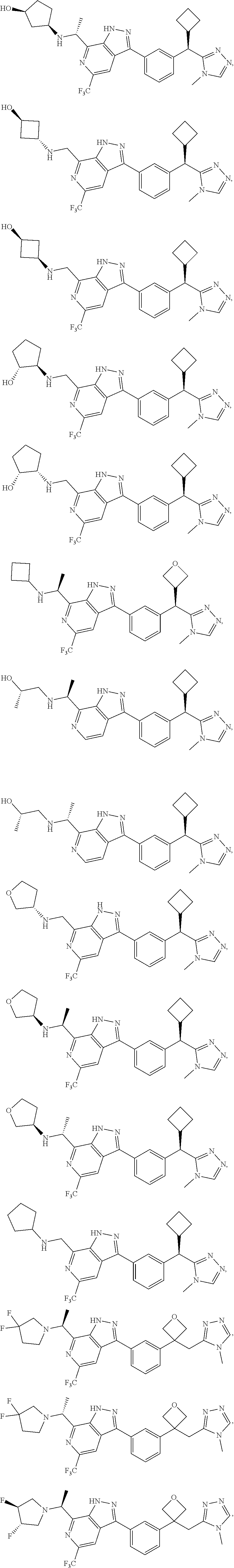

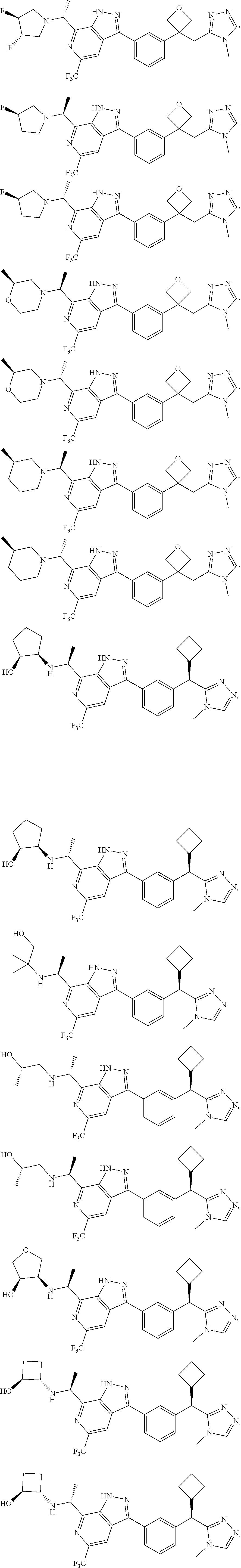

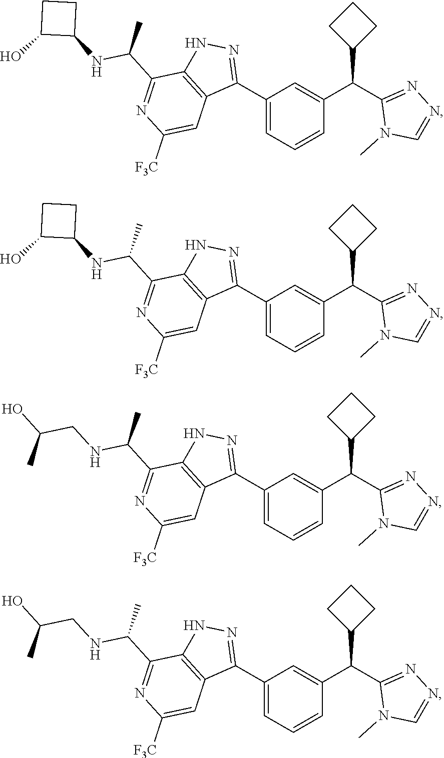

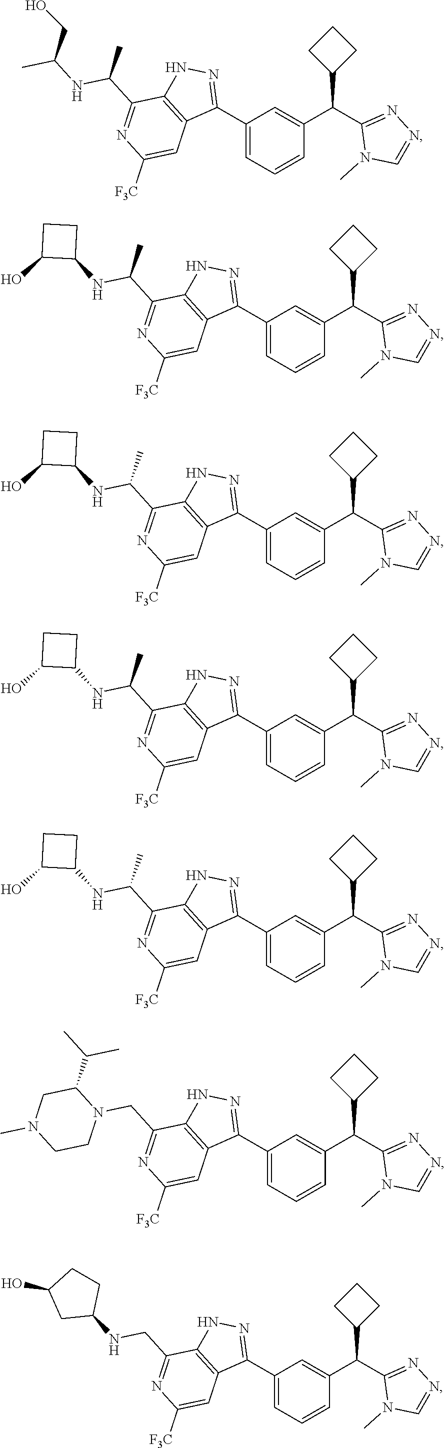

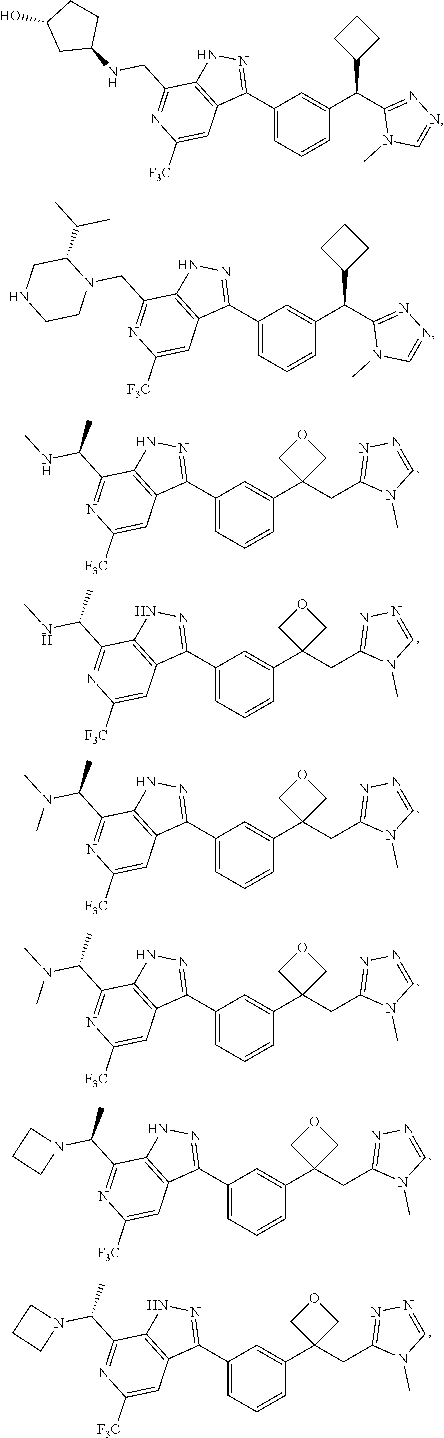

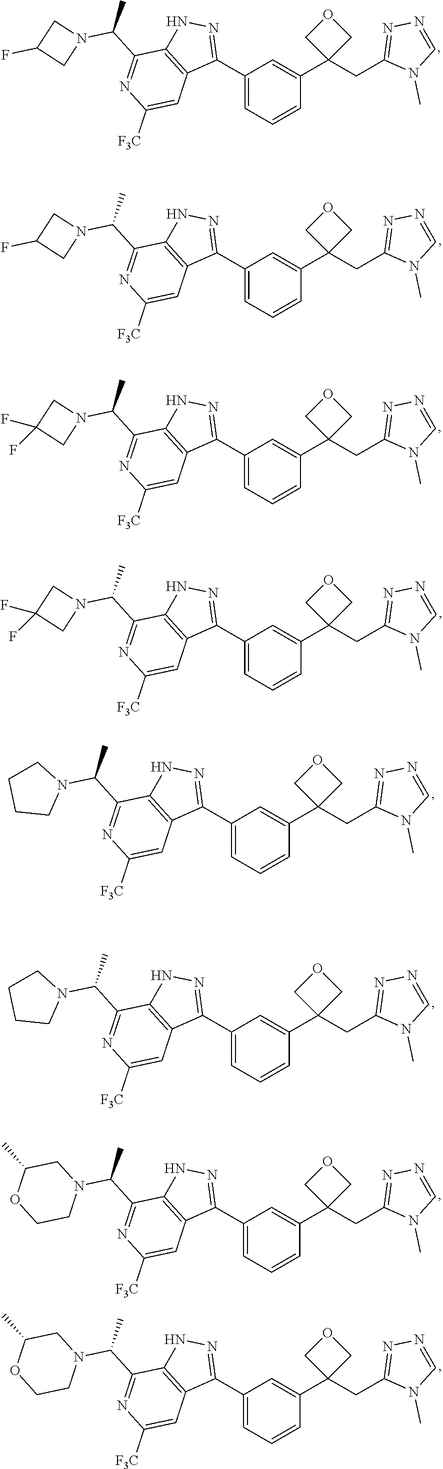

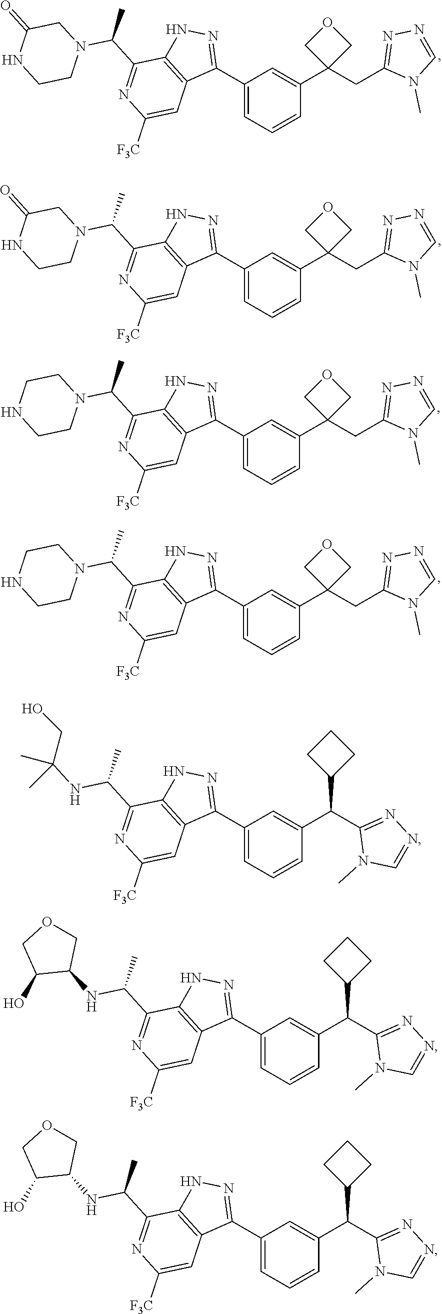

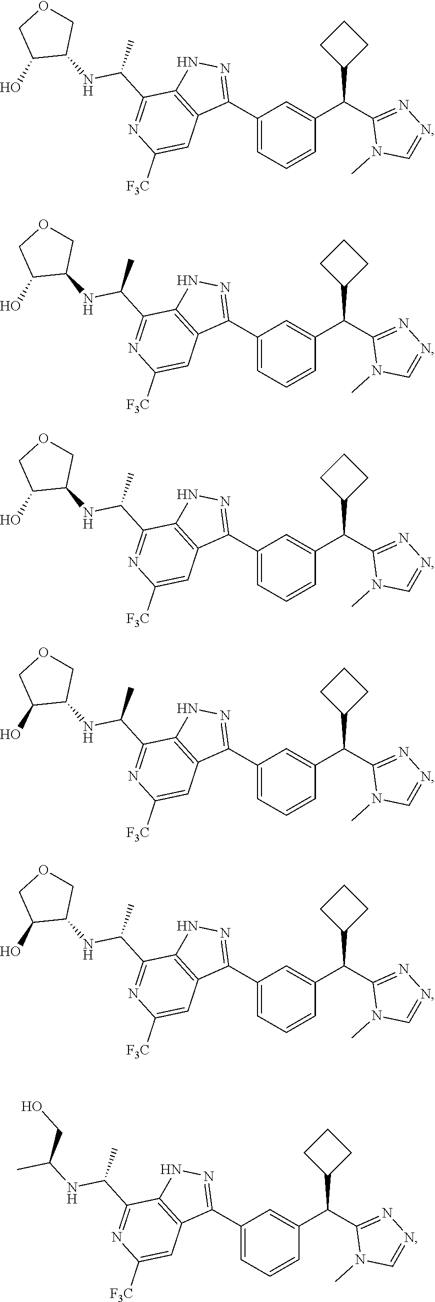

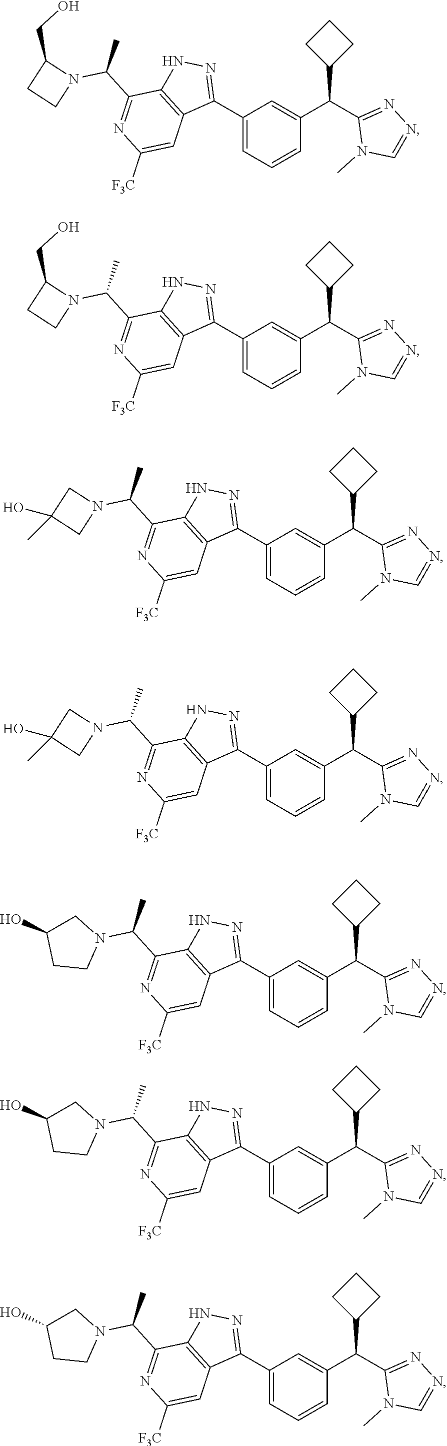

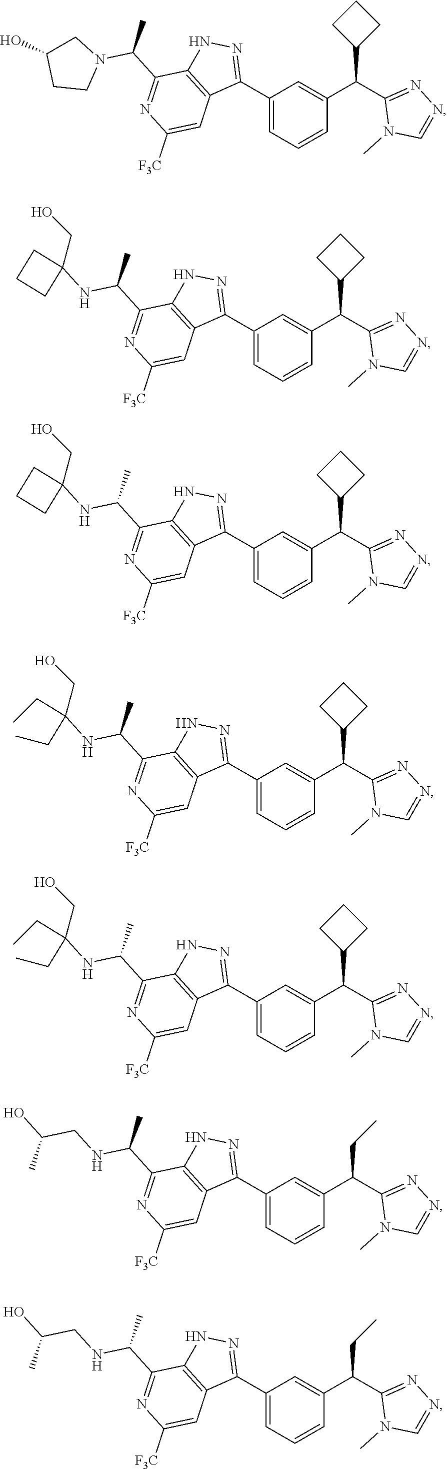

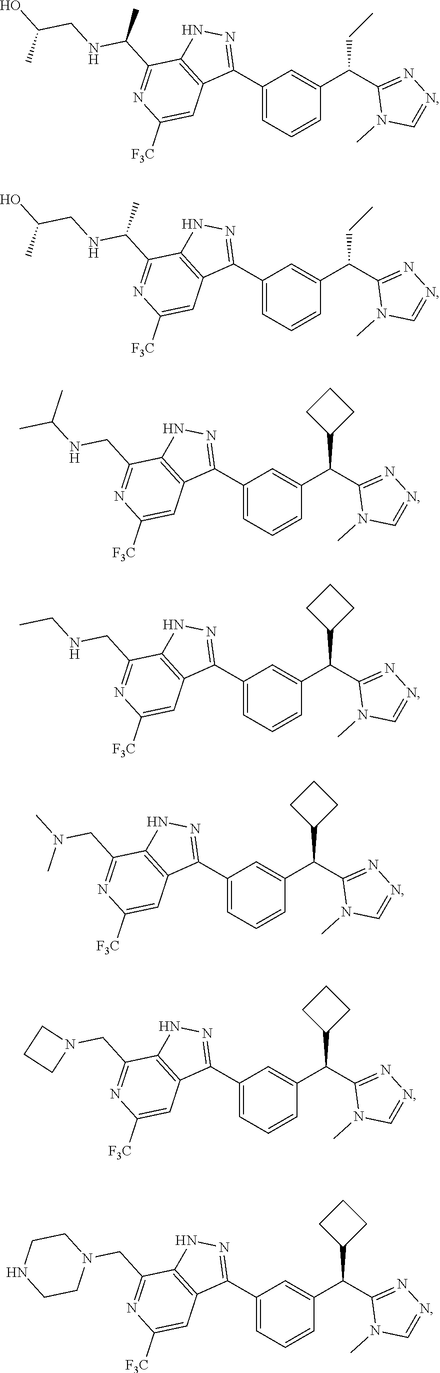

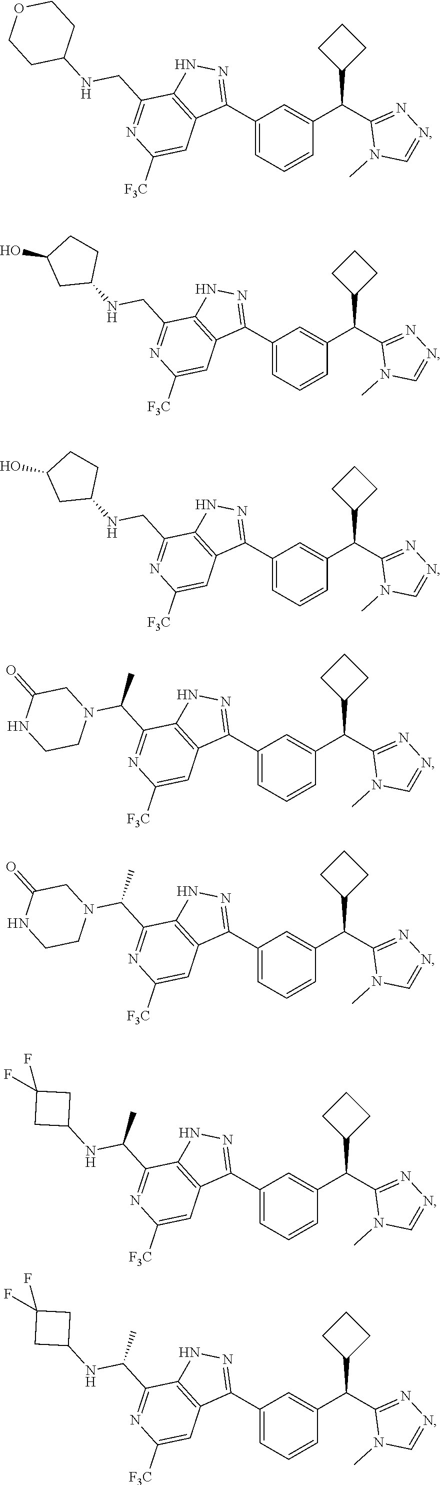

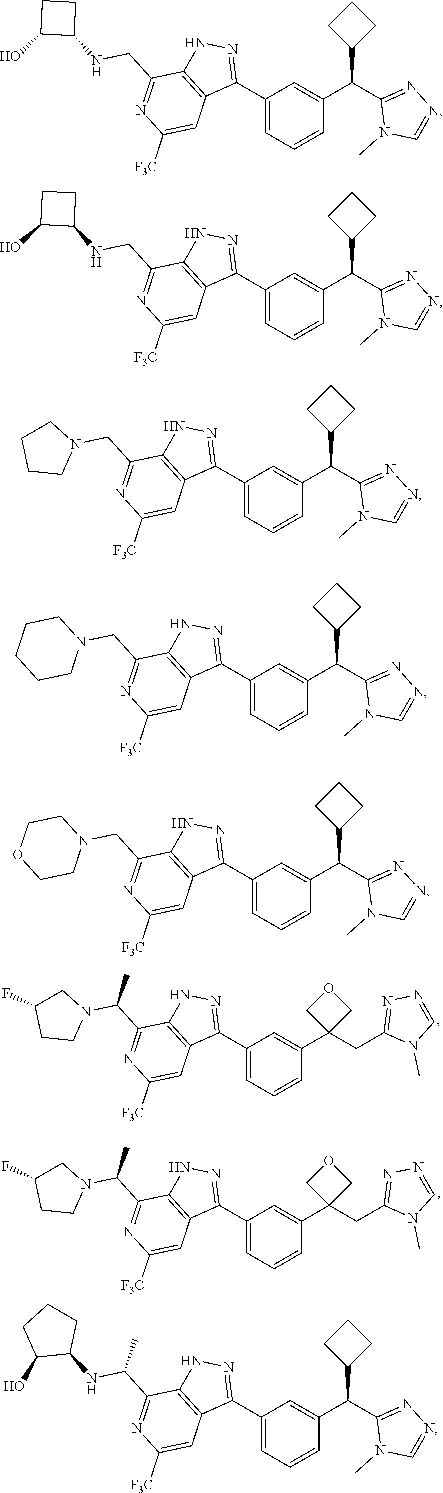

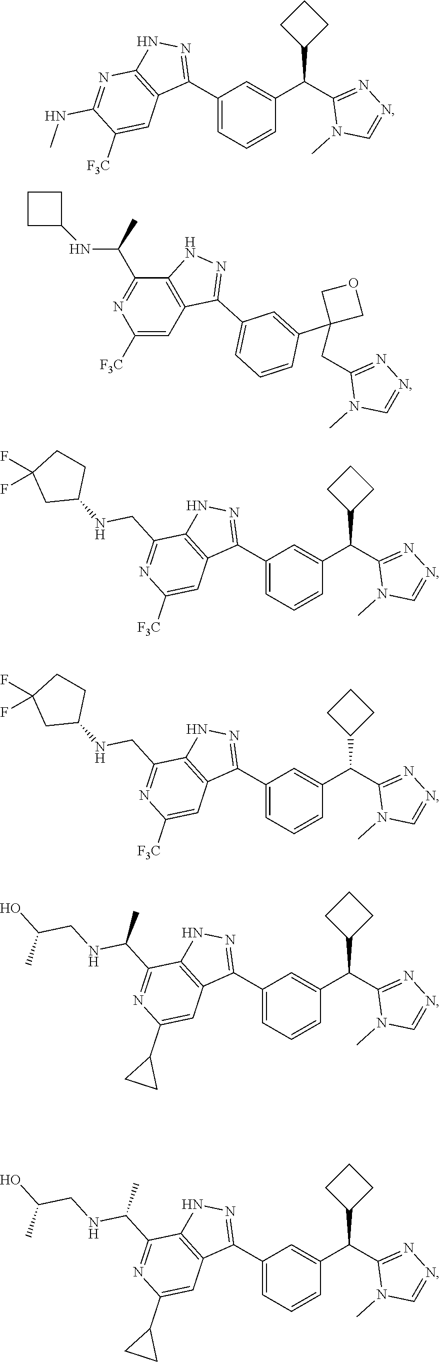

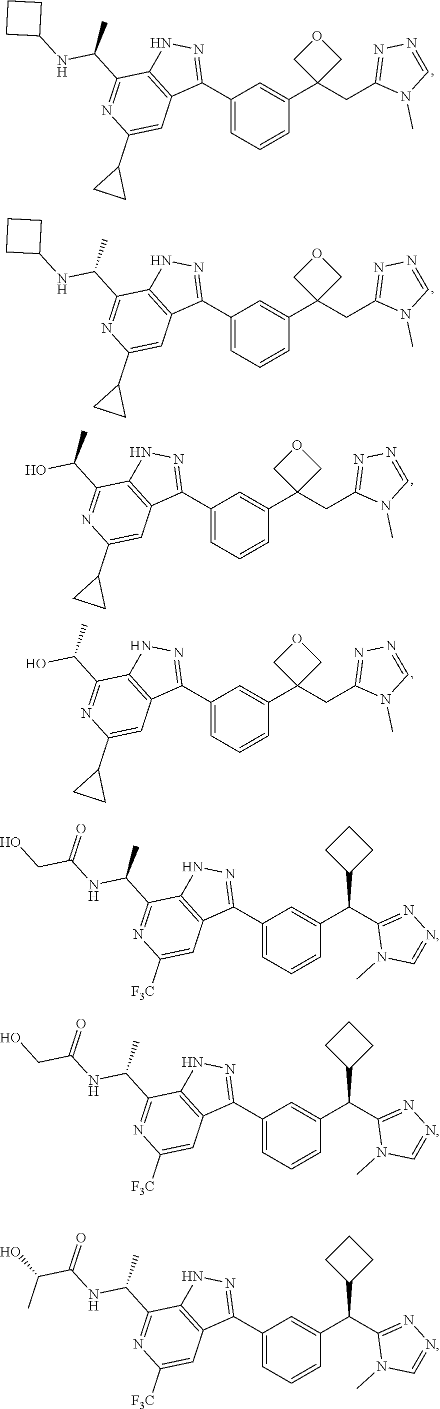

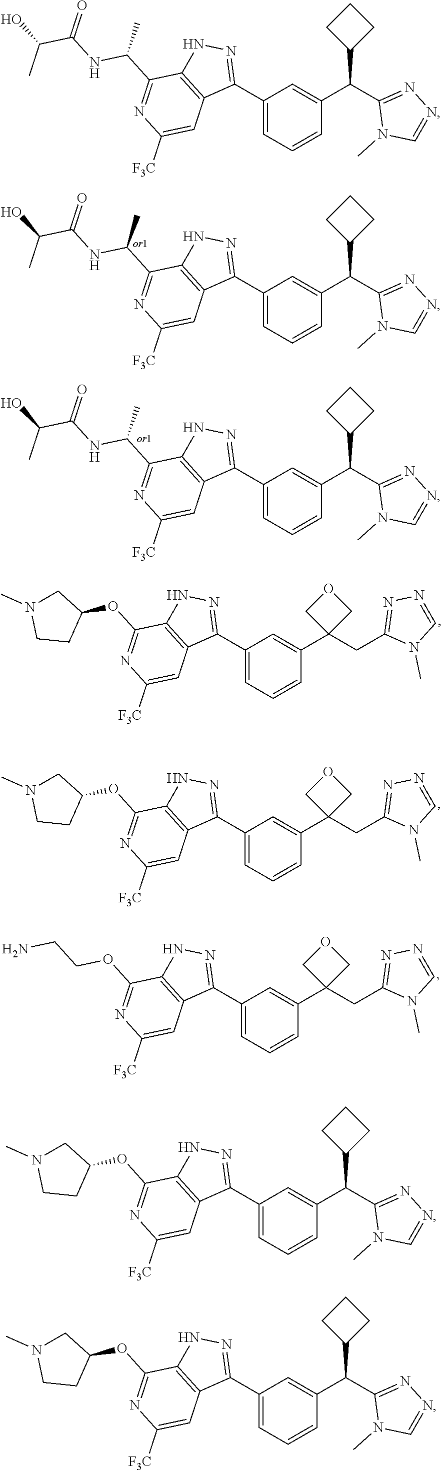

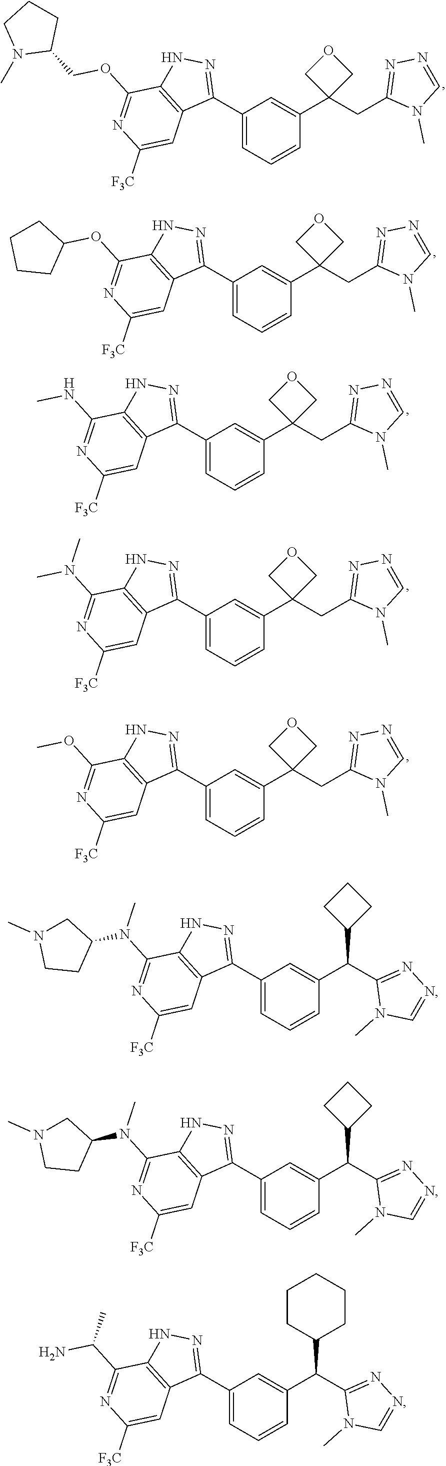

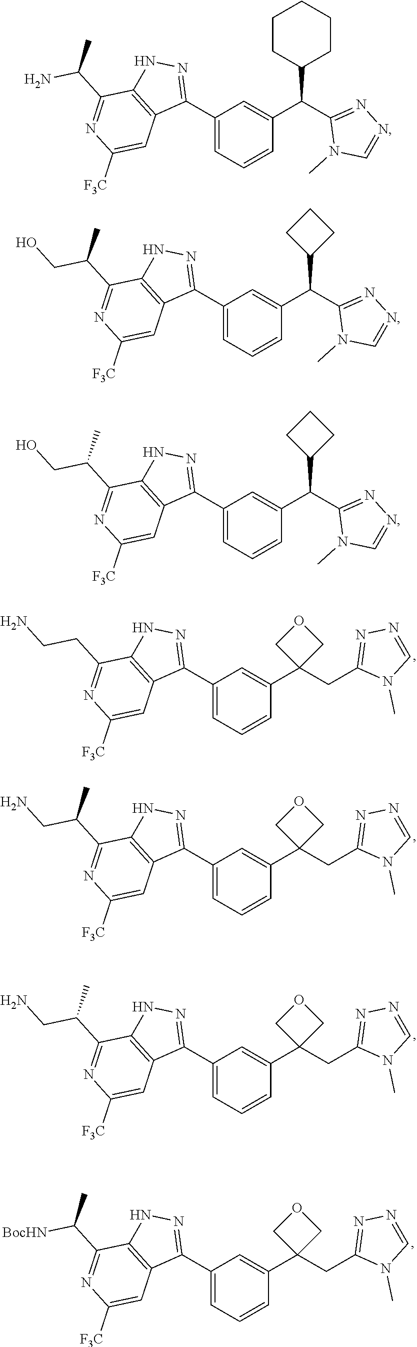

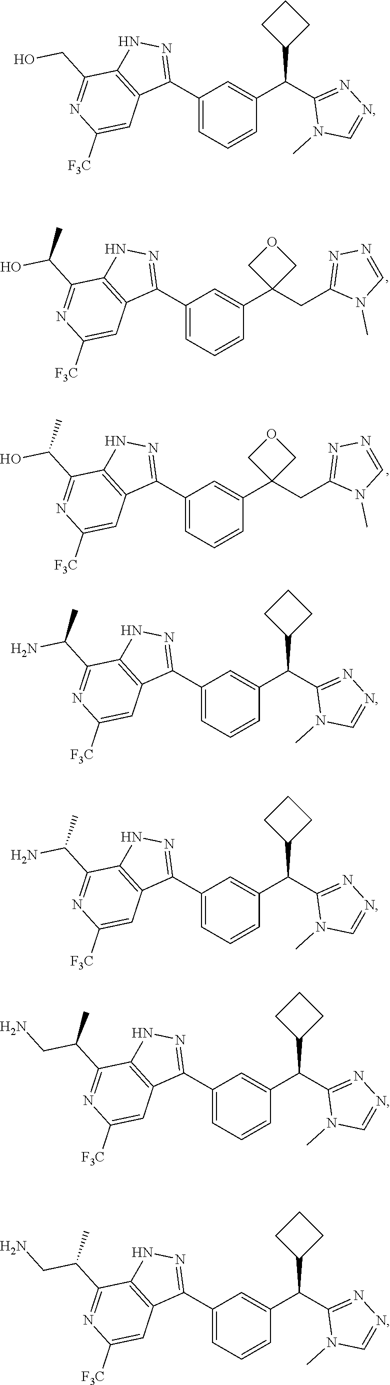

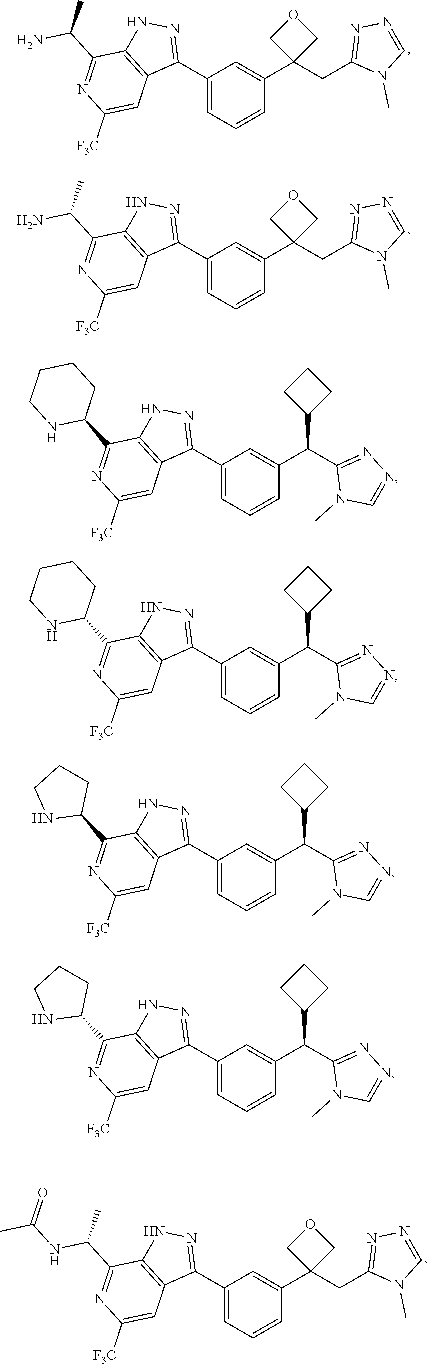

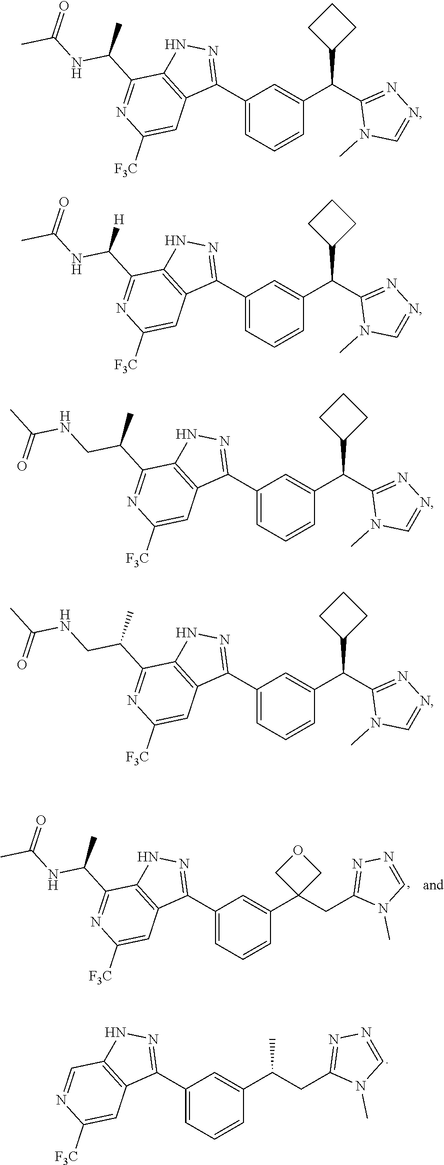

22. (canceled)

23. A compound or a pharmaceutically acceptable salt thereof, which is selected from the group consisting of:

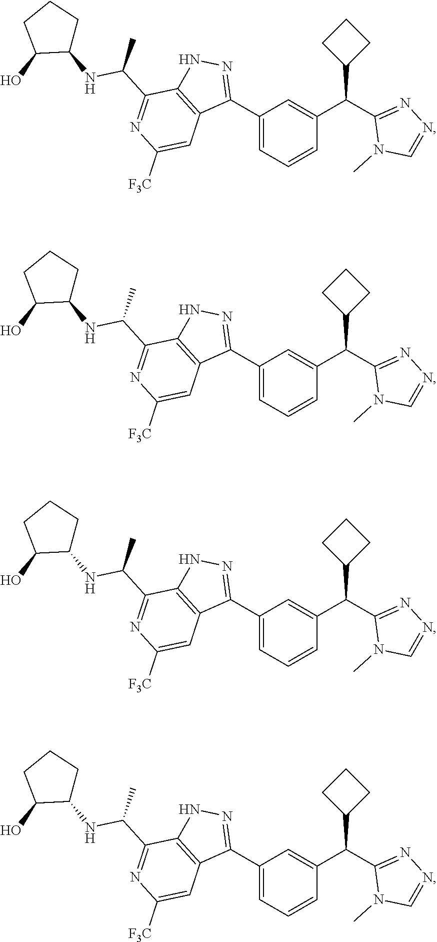 [SMILES: C[C@H](N[C@@H]1CCC[C@@H]1O)c1nc(C(F)(F)F)cc2c(-c3cccc([C@H](c4nncn4C)C4CCC4)c3)n[nH]c12 ; C[C@@H](N[C@@H]1CCC[C@@H]1O)c1nc(C(F)(F)F)cc2c(-c3cccc([C@H](c4nncn4C)C4CCC4)c3)n[nH]c12 ; C[C@H](N[C@H]1CCC[C@@H]1O)c1nc(C(F)(F)F)cc2c(-c3cccc([C@H](c4nncn4C)C4CCC4)c3)n[nH]c12 ; C[C@@H](N[C@H]1CCC[C@@H]1O)c1nc(C(F)(F)F)cc2c(-c3cccc([C@H](c4nncn4C)C4CCC4)c3)n[nH]c12]

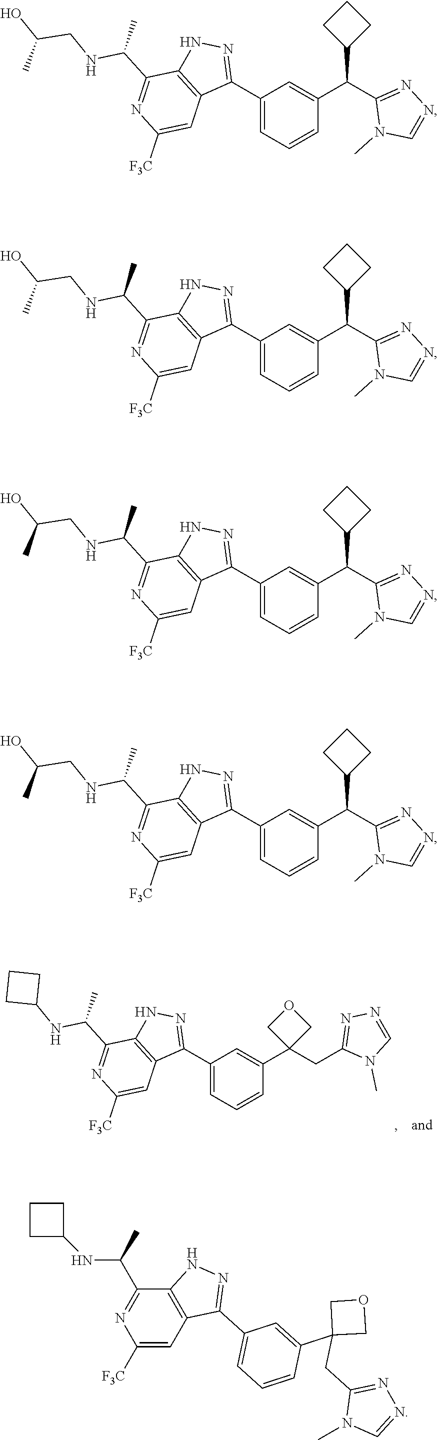 [SMILES: C[C@H](O)CN[C@H](C)c1nc(C(F)(F)F)cc2c(-c3cccc([C@H](c4nncn4C)C4CCC4)c3)n[nH]c12 ; C[C@H](O)CN[C@@H](C)c1nc(C(F)(F)F)cc2c(-c3cccc([C@H](c4nncn4C)C4CCC4)c3)n[nH]c12 ; C[C@H](NC[C@@H](C)O)c1nc(C(F)(F)F)cc2c(-c3cccc([C@H](c4nncn4C)C4CCC4)c3)n[nH]c12 ; C[C@@H](O)CN[C@H](C)c1nc(C(F)(F)F)cc2c(-c3cccc([C@H](c4nncn4C)C4CCC4)c3)n[nH]c12 ; C[C@@H](NC1CCC1)c1nc(C(F)(F)F)cc2c(-c3cccc(C4(Cc5nncn5C)COC4)c3)n[nH]c12 ; C[C@H](NC1CCC1)c1nc(C(F)(F)F)cc2c(-c3cccc(C4(Cc5nncn5C)COC4)c3)n[nH]c12]

24. A pharmaceutical composition comprising the compound according to claim 1, or a pharmaceutically acceptable salt thereof, and at least one pharmaceutically acceptable excipient.

25. A method for treating cancer in a subject in need thereof, comprising administering to the subject a therapeutically effective amount of the compound of claim 1, or a pharmaceutically acceptable salt thereof, and optionally further comprising administering a therapeutically effective amount of an additional anticancer therapeutic agent.

26. (canceled)

27. (canceled)

28. (canceled)

29. (canceled)

30. A method for the treatment of a disorder mediated by inhibition of CBL-B in a subject, comprising administering to the subject in need thereof a compound of claim 1, or a pharmaceutically acceptable salt thereof, in an amount that is effective for treating the disorder.

31. A pharmaceutical combination comprising a compound of claim 1 or a pharmaceutically acceptable salt thereof, and at least one additional therapeutic agent or a pharmaceutically acceptable salt thereof.

32. (canceled)
